(19) United States
(12) Patent Application Publication
(10) Pub. No.: US 2025/0074911 A1
(43) Pub. Date: Mar. 6, 2025

(54) MACROCYCLIC BTK INHIBITORS

(71) Applicant: Crossfire Oncology Holding B.V., Oss (NL)

(72) Inventors: Adrianus Petrus Antonius de Man, Hurwenen (NL); Rogier Christian Buijsman, Berghem (NL); Jan Gerard Sterrenburg, Renkum (NL); Joeri Johannes Petrus de Wit, Boekel (NL); Freek van Cauter, Lent (NL); Sander Petrus Wilhelmus van Gemert, s-Hertogenbosch (NL); Martine Berendina Wilhemina Prinsen, s-Hertogenbosch (NL); Winfried Robert Mulder, Nijmegen (NL); Michelle Muller, Scherpenzeel (NL); Diep Vu-Pham, Oss (NL); Yvonne Grobben, Den Bosch (NL); Wilhelmina Elisabeth Simons-van Riel, Nuenen (NL); Yvonne Gertruda Theodora Hendrika Van Mil, Heesch (NL)

(73) Assignee: Crossfire Oncology Holding B.V., Oss (NL)

(21) Appl. No.: 18/718,462

(22) PCT Filed: Dec. 14, 2022
(86) PCT No.: PCT/EP2022/085765

§ 371 Date: Jun. 11, 2024

(30) Foreign Application Priority Data
Dec. 14, 2021 (WO) PCT/EP2021/085641
Dec. 14, 2021 (WO) PCT/EP2021/085645
Dec. 13, 2022 (WO) PCT/EP2022/085713

(51) Int. Cl. C07D 471/14; A61K 31/444; A61K 31/4985; A61K 45/06; C07D 471/22; C07D 487/14; C07D 498/14; C07D 513/14

(52) U.S. Cl. CPC C07D 471/14; A61K 31/444; A61K 31/4985; A61K 45/06; C07D 471/22; C07D 487/14; C07D 498/14; C07D 513/14

(57) ABSTRACT

The present invention relates to macrocyclic compounds and compositions containing said compounds acting as kinase inhibitors, in particular as inhibitors of BTK (Bruton's Tyrosine Kinase). Moreover, the present invention provides processes for the preparation of the disclosed compounds, as well as methods of using them, for instance as a medicament, in particular for the treatment of Bruton's Tyrosine Kinase (BTK) mediated disorders, such as cancer.

### FIELD OF THE INVENTION

The present invention relates to compounds. More specifically, the invention relates to macrocyclic compounds and compositions for use as kinase inhibitors, along with processes to prepare the compounds and uses of the compounds. Specifically, the invention relates to reversible macrocyclic BTK inhibitors. More in particular, the invention relates to reversible macrocyclic wild-type and mutant-BTK inhibitors with long target residence time.

### BACKGROUND OF THE INVENTION

Kinases are enzymes that transfer a phosphate group from ATP to a protein while phosphatases remove a phosphate group from protein. Together, these two enzymatic processes regulating cellular functions such as cell proliferation, subcellular translocation, apoptosis, inflammation and metabolism (Attwood M. M. et al (2021) Nat Rev Drug Discov). The human kinome is composed of over 500 kinases. The recent development of small-molecule kinase inhibitors for the treatment of diverse types of cancer has proven successful in clinical therapy.

Bruton's tyrosine kinase (BTK) is a member of the Src-related Tec family of protein kinases which are a large subset of kinases which play a central role in the regulation of a wide variety of cellular signaling processes. BTK plays a key role in the B-cell receptor signaling and a critical role in the regulation of survival, proliferation, activation and differentiation of B-lineage cells. Targeting of BTK with small molecule inhibitors such as the FDA approved irreversible BTK inhibitors ibrutinib, acalabrutinib, zanubrutinib and tirabrutinib has proven to be efficacious in several B cell malignancies including Chronic Lymphocytic Leukemia (CLL), Mantle Cell Lymphoma (MCL), Waldenström's Macroglobulinemia (WM) and Small Lymphocytic Lymphoma SLL. Combinations of BTK inhibitors with other novel drugs or regimens results in more profound responses and much higher rates of minimal residual disease negativity.

BTK is also expressed and plays also pro-tumorigenic roles in several solid tumors (Xianhui Wang et al. 2021). In prostate cancer cells BTK inhibition with ibrutinib or acalabrutinib inhibited cell growth (Kokabee et al 2015). Ibrutinib has also been shown to inhibit in vivo (xenograft) breast cancer cell growth (Wang et al., 2016) and inhibition of BTK with ibrutinib blocked gastric cancer cell growth (Wang et al., 2016). BTK inhibitors have also showed inhibition of cellular proliferation and migration, and induced apoptosis and autophagy in glioblastoma cell lines (Wei et al., 2016; Wang et al., 2017).

In addition to its role in BCR signaling, BTK is also involved in many other immunological pathways which provides a rationale for the targeting of BTK in the context of inflammatory and systemic autoimmune disease (Stefan F. H. Neys et al. 2021).

A drawback of the currently approved irreversible inhibitors is that drug resistance in malignant diseases can develop when BTK variations at the catalytic site and the gatekeeper of the BTK are not able to bind efficiently to irreversible inhibitors in patients treated with currently approved BTK inhibitors. This is a rather common event in patients treated with irreversible inhibitors and who experience relapse. A major mechanism for the acquired resistance is the emergence of BTK cysteine 481 (C481) mutations. These mutations hamper binding of irreversible inhibitors such as ibrutinib and acalabrutinib which form a covalent bond with this amino acid. Other mutations that can result in acquired resistance to both irreversible covalent and reversible non-covalent BTK inhibitors are BTK gatekeeper residue threonine 474 (T474) mutations which can reduce BTK inhibitor access to BTK (Rula Zain et al. 2021, Shenqiu Wang et al. 2019).

Second-generation BTK inhibitors include acalabrutinib, zanubrutinib, and tirabrutinib which offer greater BTK selectivity. While these agents may limit off-target toxicity, they do not overcome common mechanisms of ibrutinib resistance. Reversible BTK inhibitors including vecabrutinib, LOXO-305 (pirtobrutinib) and ARQ-531 (nemtabrutinib) inhibit BTK in the presence of C481S mutation. Vecabrutinib is disclosed in WO2013/185084, pirtobrutinib was first disclosed in WO2017/103611 and ARQ-531 was disclosed in WO2017/111787

Further reversible BTK inhibitors are disclosed in WO2017/046604, WO2020/015735, WO2020/239124, WO2021/093839, WO2020/043638, WO2013/067274, WO2018097234, WO2013/010380, WO2016/161570, WO2016/161571, WO2016/106624, WO2016/106625, WO2016/106626, WO2016106623, WO2016/106628 and WO2016/109222.

Kinase activity is commonly assessed by measuring the half-maximum inhibitory potency (IC₅₀) or binding affinity (K_{D}) in a kinase enzyme activity assay or using Surface Plasmon Resonance (SPR) (Willemsen-Seegers N. et al (2016) J Mol Biol). An important aspect of these assays is that IC₅₀ or K_{D} is measured in a system with a fixed concentration of inhibitor. In living systems, compounds and enzymes usually encounter each other in a compartment, such as the cell, or at the cell surface, where the compound diffuses in and out, or may be actively extruded by drug pumps. Thus, whereas the concentration of a target remains constant, the concentration of compound is continually changing. In such a system, it has been proposed that the time a compound resides on its target, the target residence time tau (T), is a more important determinant of its pharmacological activity than the IC₅₀ or K_{D} measured at equilibrium (Copeland R. A. et al (2006) Nat Rev Drug Discov). In addition, if a drug demonstrates a long residence time on its target and a short residence time on its off-targets, selectivity is enhanced, providing advantages for drug safety (Barf T. and Kaptein A. (2012) J Med Chem). Thus, the biological action of drugs with long target residency can endure long after it is cleared from the systemic circulation. An extreme example of drugs with long target residence time is irreversible inhibitors, usually obtained by covalent binding to the target. Compounds with equipotent affinity or potency might possess different residence times on their target protein as was demonstrated by erlotinib, gefitinib and lapatinib on EGFR (Willemsen-Seegers N. et al (2016) J Mol Biol).

Therefore, an aim of the present invention is to provide BTK inhibitors providing improved pharmacological activity. In addition, the invention aims to provide mutant-BTK inhibitors suitable for inhibiting BTK mutant(s).

It is an aim of certain embodiments of this invention to provide compounds which inhibit the BTK T474I mutant kinase.

Furthermore, it is an aim of certain embodiments of this invention to provide reversible (mutant) BTK inhibitors with a long target residence time.

Another aim of certain embodiments of this invention is to provide cancer treatments. In particular, it is an aim of certain embodiments of this invention to provide compounds with have comparable (BTK) activity to existing cancer treatments but are also effective against mutations.

One of the aspects of the invention focus on providing BTK inhibitors effective against the C481, T316 or T474 mutations.

### SUMMARY OF THE INVENTION

The inventors have found that a compound of Formula (I-a) to (I-h):

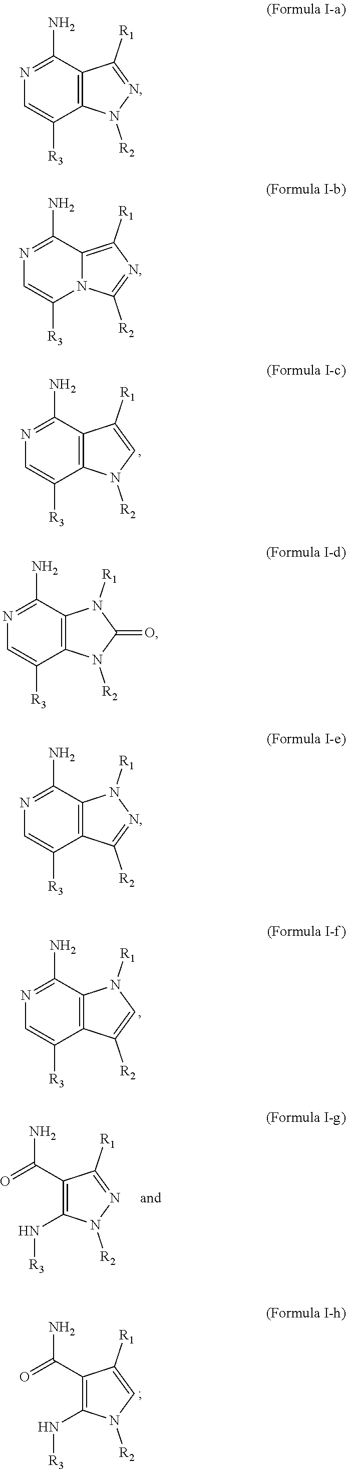

wherein said compound is as further described below, or a pharmaceutically acceptable salt and/or solvate thereof, provides improved BTK inhibition. The inventors have found that these compounds being a macrocyclic compound having any one of the scaffolds of Formula (I-a) to (I-h) provide improved BTK inhibition.

In a first aspect of the invention is provided a compound of Formula (I-a) to (I-h) or a pharmaceutically acceptable salt and/or solvate thereof, wherein the compound is selected from the group consisting of:

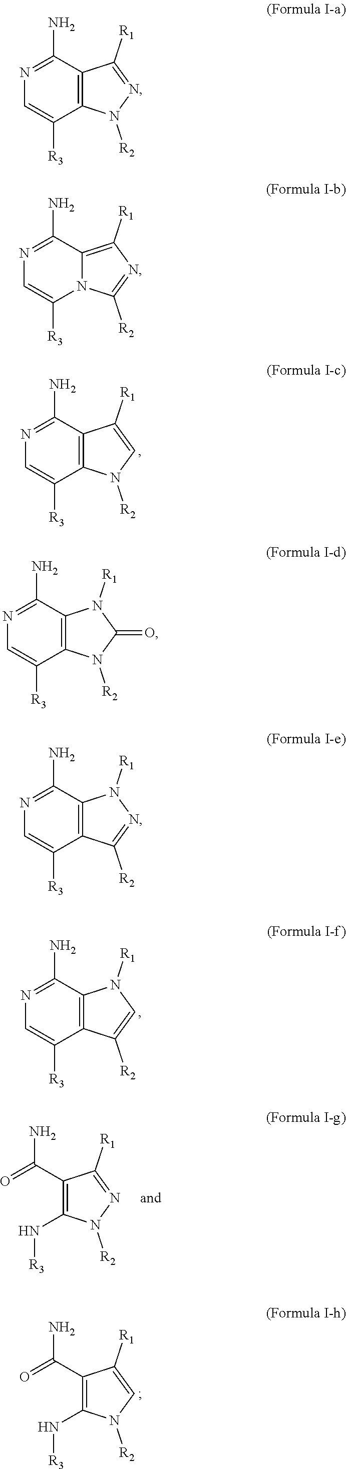

Wherein R¹ is

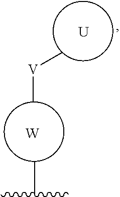

wherein:

W is an aryl group having 6-10 carbon or a heteroaryl group having 1-5 carbon, wherein any said aryl group and heteroaryl group is optionally and independently substituted with one or more substituents selected from halogen, (1-2C)alkyl, (1-2C)alkoxy; wherein any of said alkyl and alkoxy group is optionally and independently substituted with one, two or three fluoro; V is any one of O, —C(O)—NH—, —NH—C(O)—, —CH(R^{1v})—NH—C(O)—, —CH(R^{1v})—; R^{1v} is hydrogen or (1-2C)alkyl;

U is an aryl group having 6-10 carbon or an heteroaryl group having 1-5 carbon; wherein any of said aryl group and heteroaryl group is optionally and independently substituted with one or more substituents selected from halogen, cyano, (1-4C)alkyl, (1-5C)alkoxy, (3-6C)cycloalkyl or (3-6C)heterocycloalkyl; wherein any of said alkyl, alkoxy, cycloalkyl and heterocycloalkyl group is optionally and independently substituted with one, two or three halogen;

wherein R² is of Formula (II-a) to (II-f) selected from the group consisting of:

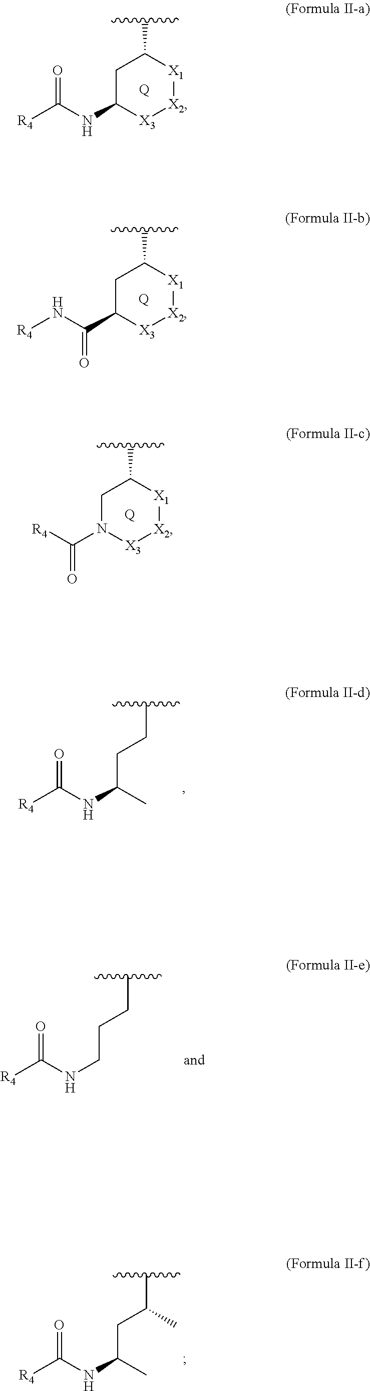

wherein Q is a monocyclic ring selected from a (3-7C)cycloalkyl and a (3-6C)heterocycloalkyl, wherein X₁, X₂ and X₃ are independently selected from CH₂, —CH₂CH₂—, O, N and a direct bond; wherein any of the cycloalkyl, heterocycloalkyl and alkyl group is optionally and independently substituted with one or more substituents selected from halogen, hydroxy, (1-3C)alkyl, (1-3C)alkoxy, (1-4C)alkylcarbonyl or (3-4C)cycloalkyl; wherein any of said alkyl and alkoxy group is optionally and independently substituted with one, two or three halogen;

R³ and R⁴ together represent a linker having Formula (III-1) to (III-40) selected from the group consisting of:

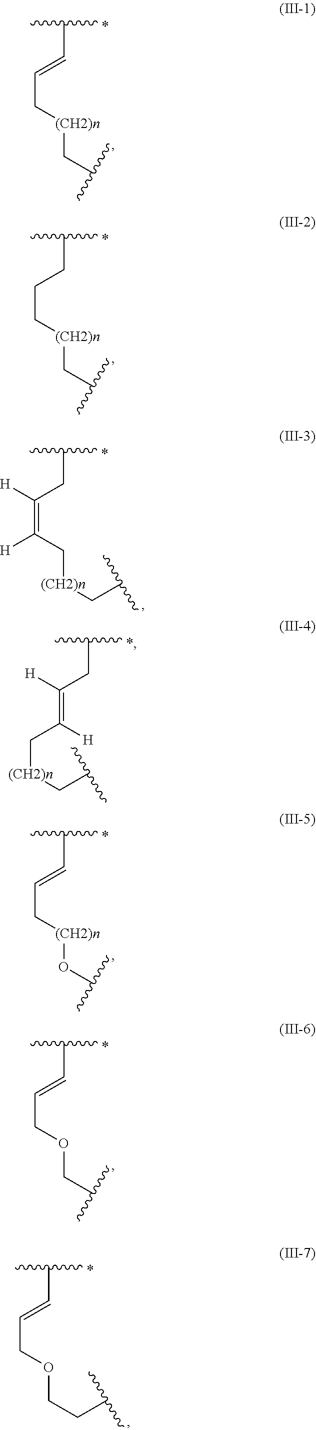

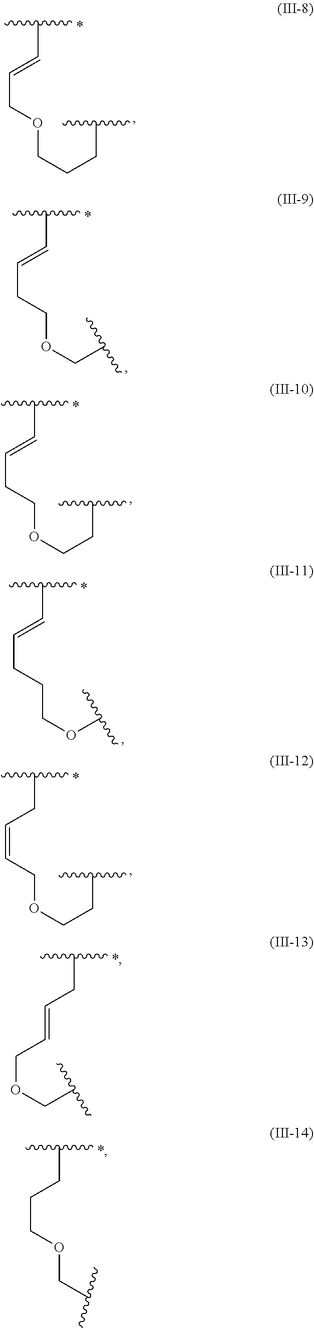

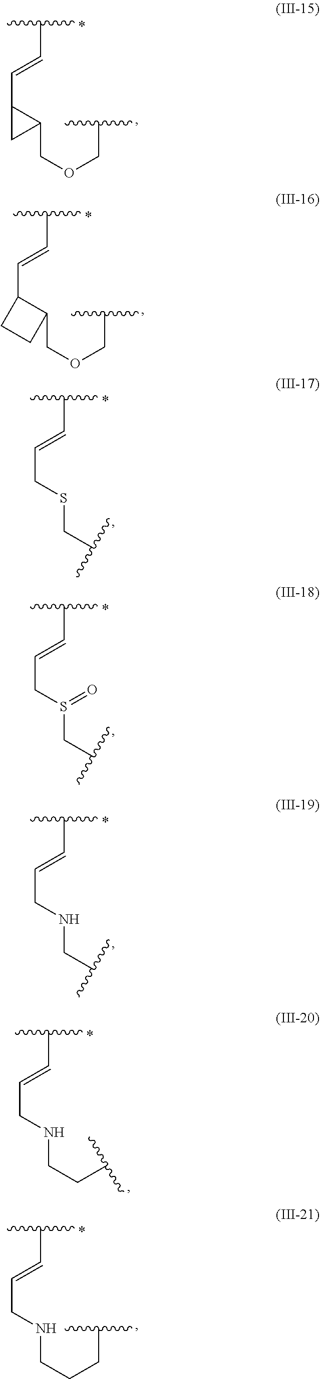

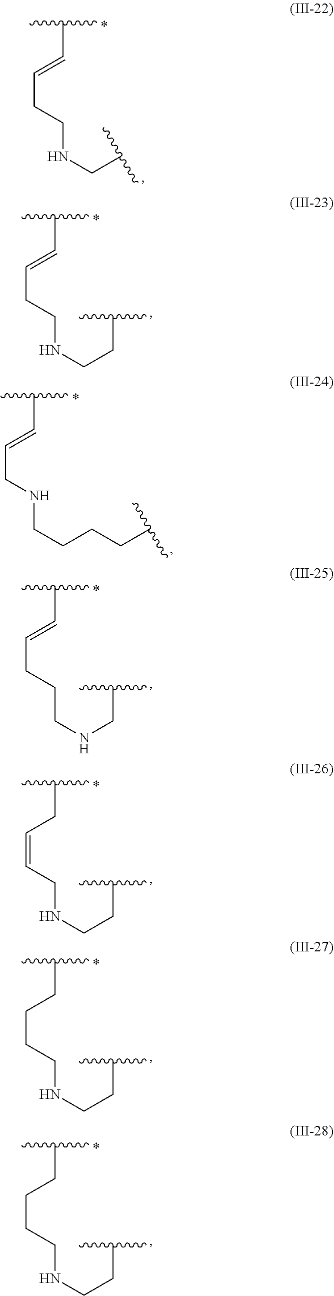

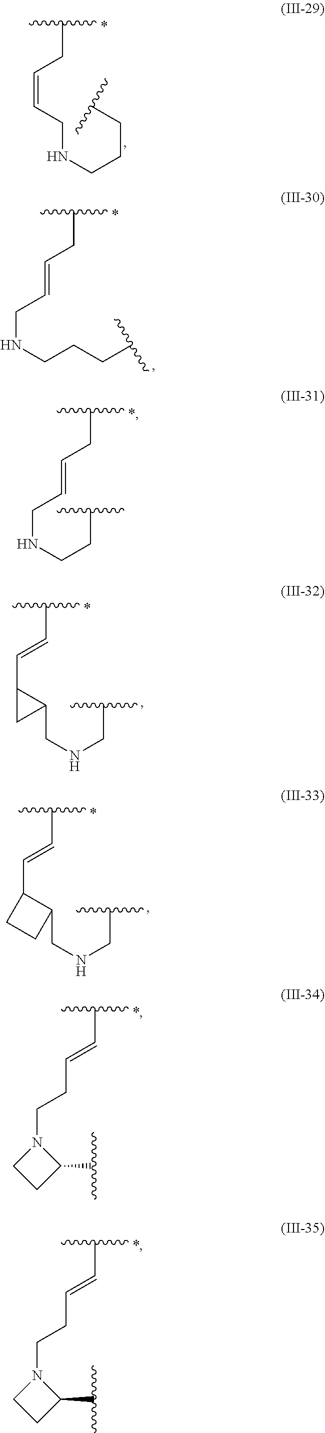

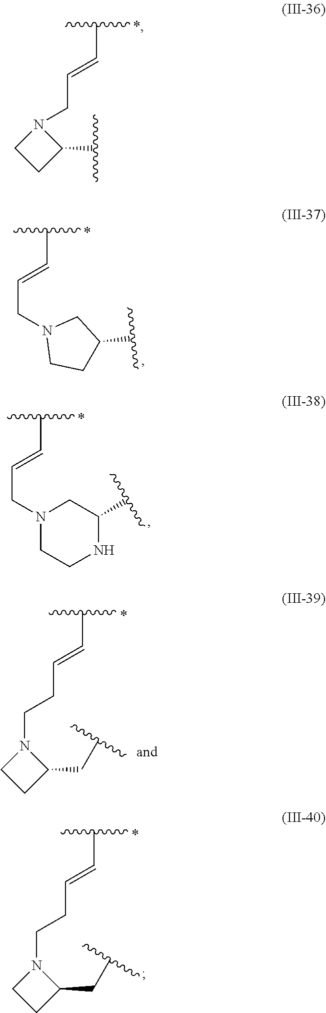 [SMILES: */C=C/CN1CC[C@H]1* ; */C=C/CN1CC[C@@H](*)C1 ; */C=C/CN1CCN[C@@H](*)C1 ; */C=C/CCN1CC[C@H]1C* ; */C=C/CCN1CC[C@@H]1C*]

whereby the

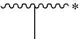

marks the position of R³ in any one of Formula I-a to I-h, and whereby the

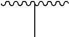

marks the position of R⁴ in any one of Formula II-a to II-f;

wherein any of said linkers is optionally and independently substituted with one or more substituents selected from deuterium, halogen, oxo, hydroxy, CD₃, (1-4C)alkyl, (1-5C)alkoxy, (3-6C)cycloalkyl, (3-6C)cycloalkoxy and (1-6C)alkylcarbonyl; wherein any of said alkyl and alkoxy group is optionally and independently substituted with one, two or three halogen.

In a second aspect of the invention is provided a compound according to the invention or a pharmaceutically acceptable salt thereof, for use as a medicament.

In another aspect of the invention is provided a compound according to the invention or a pharmaceutically acceptable salt thereof, for use in therapy.

In another aspect of the invention is provided a compound according to the invention or a pharmaceutically acceptable salt thereof, for use in the treatment of Bruton's Tyrosine Kinase (BTK) mediated disorders.

In another aspect of the invention is provided a compound of according to the invention or a pharmaceutically acceptable salt thereof, for use in the treatment of cancer.

In another aspect of the invention is provided a use of the compound according to the invention or a pharmaceutically acceptable salt thereof, for the manufacture of a medicament.

In another aspect of the invention is provided a pharmaceutical composition which comprises the compound according to the invention or a pharmaceutically acceptable salt thereof and one or more pharmaceutically acceptable excipients.

In another aspect of the invention is provided a method for treating of cancer in a subject in need thereof comprising administering to the subject the compound according to the invention or a pharmaceutically acceptable salt thereof in an amount effective to treat cancer.

In another aspect of the invention is provided a method for treating a subject suffering with a Bruton's Tyrosine Kinase (BTK) mediated disorder comprising administering to the subject the compound of the invention in an amount effective to treat the BTK mediated disorder.

Each of the sub-formulas 1-226 of the compound is a preferred embodiment of the present application.

The present invention will be illustrated further by means of the following non-limiting examples.

### Definitions

The term “pharmaceutical composition” as used herein has its conventional meaning and refers to a composition which is pharmaceutically acceptable.

The term “pharmaceutically acceptable” as used herein has its conventional meaning and refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio.

The term “effective amount” as used herein, refers to an amount of the compound of the invention, and/or an additional therapeutic agent, or a composition thereof, that is effective in producing the desired therapeutic, ameliorative, inhibitory or preventive effect when administered to a subject suffering from a BTK-mediated disease or disorder. In the combination therapies of the present invention, as effective amount can refer to each individual agent or to the combination as a whole, wherein the amounts of all agents administered are together effective, but wherein the component agent of the combination may not be present individually in an effective amount.

A “subject” is a human or non-human mammal. In one embodiment, a subject is a human.

The term “controlling” is intended to refer to all processes wherein there may be a slowing, interrupting, arresting or stopping of the progression of the diseases and conditions affecting the mammal. However, “controlling” does not necessarily indicate a total elimination of all disease and condition symptoms, and is intended to include prophylactic treatment.

The term “excipient” as used herein has its conventional meaning and refers to a pharmaceutically acceptable ingredient, which is commonly used in the pharmaceutical technology for preparing a granulate, solid or liquid oral dosage formulation.

The term “salt” as used herein has its conventional meaning and includes the acid addition and base salts of the compound of the invention.

The term “solvate” as used herein has its conventional meaning. One or more compounds of the invention or the pharmaceutically acceptable salts thereof may exist in unsolvated as well as solvated forms with pharmaceutically acceptable solvents such as water, ethanol, and the like. “Solvate” means a physical association of a compound of this invention with one or more solvent molecules. This physical association Involves varying degrees of ionic and covalent bonding. Including hydrogen bonding. In certain instances the solvate will be capable of isolation, for example when one or more solvent molecules are incorporated in the crystal lattice of the crystalline solid. “Solvate” encompasses both solution-phase and isolatable solvates. Examples of suitable solvates include ethanolates, methanolates, and the like. “Hydrate” is a solvate wherein the solvent molecule is H₂O and includes any hydrate of the compound or the salt of said compound.

The term “treatment” as used herein has its conventional meaning and refers to curative, palliative and prophylactic treatment.

The term “unit dosage form” has its conventional meaning and refers to a dosage form which has the capacity of being administered to a subject, preferably a human, to be effective, and which can be readily handled and packaged, remaining as a physically and chemically stable unit dose comprising the therapeutic agent, i.e. the compound of the invention.

The term “BTK” as used herein has its conventional meaning and refers to Bruton's Tyrosine Kinase. Bruton's tyrosine kinase (BTK) is a member of the Src-related Tec family of protein kinases which are a large subset of kinases which play a central role in the regulation of a wide variety of cellular signaling processes. BTK plays a key role in the B-cell receptor signaling and a critical role in the regulation of survival, proliferation, activation and differentiation of B-lineage cells. Targeting of BTK with small molecule inhibitors such as the FDA approved irreversible BTK inhibitors ibrutinib, acalabrutinib, zanubrutinib and tirabrutinib has proven to be efficacious in several B cell malignancies including Chronic Lymphocytic Leukemia (CLL), Mantle Cell Lymphoma (MCL), Waldenström's Macroglobulinemia (WM) and Small Lymphocytic Lymphoma SLL. Combinations of BTK inhibitors with other novel drugs or regimens results in more profound responses and much higher rates of minimal residual disease negativity.

The term “BTK inhibitor” as used herein has its conventional meaning and refers to an inhibitor for BTK. A BTK inhibitor may be a small molecule inhibitor. Inhibitors may be irreversible inhibitors, such as by forming a covalent bond, and may be reversible inhibitors, which may form a temporary interaction with BTK.

The term “mutant-BTK” as used herein has its conventional meaning and refers to mutations of BTK. Mutations of BTK may be referred to by an altered amino acid target (such as C as single-letter data-base code for cysteine) at a certain position of the BTK structure (such as 481). Additionally, the amino acid substitution at the mutation position may be referred to by an additional amino acid single-letter data-base code, such as C481 S for serine substitution and C481T for threonine substitution of cysteine at the 481 position.

A drawback of the currently approved irreversible inhibitors is that drug resistance in malignant diseases can develop when BTK variations at the catalytic site and the gatekeeper of the BTK are not able to bind efficiently to irreversible inhibitors in patients treated with currently approved BTK inhibitors. This is a rather common event in patients treated with irreversible inhibitors and who experience relapse. A major mechanism for the acquired resistance is the emergence of BTK cysteine 481 (C481) mutations. These mutations hamper binding of irreversible inhibitors such as ibrutinib and acalabrutinib which form a covalent bond with this amino acid. Other mutations that can result in acquired resistance to both irreversible covalent and reversible non-covalent BTK inhibitors are BTK gatekeeper residue threonine 474 (T474) mutations which can reduce BTK inhibitor binding to BTK.

The term “wt-BTK” or “WT-BTK” or “BTK^{WT}” as used herein has its conventional meaning and refers to wild-type Bruton's Tyrosine Kinase. A wild-type BTK has the regular meaning of a phenotype of the typical form of BTK as it occurs in nature. Originally, the wild-type was conceptualized as a product of the standard “normal” allele at a locus, in contrast to that produced by a non-standard, “mutant” allele.

The term “macrocycle” as used herein has its conventional meaning and refers to a part of a molecule containing a ring consisting of 12 or more ring atoms forming said ring. In an example, a twelve membered ring consist of 12 atoms forming said ring.

The term “binding affinity (K_{D})” as used herein has its conventional meaning and refers to the equilibrium dissociation constant which is an inverse measure of the affinity of a protein-ligand (small molecule) pair under equilibrium conditions. The value of K_{D} is mathematically equivalent to the ratio k_{off}/kₒₙ (or k_{d}/kₐ) measured using Surface Plasmon Resonance (SPR).

The term “association rate constant” or “on-rate (kₒₙ or kₐ)” as used herein has its conventional meaning and refers to a second-order rate constant that quantifies the rate at which a free ligand and free protein combine (through collisional encounters) to form a binary protein-ligand complex.

The term “dissociation rate constant” or “off-rate (k_{off} or k_{d})” as used herein has its conventional meaning and refers to a first-order rate constant that quantifies the rate at which a binary protein-ligand complex dissociates to the free ligand and free protein.

The term “target residence time tau (T)” as used herein has its conventional meaning and refers to the time a compound resides on its target. Target residence time (T) can be determined according to the method as described below in the experimental section.

The term “IC₅₀” as used herein has its conventional meaning and refers to the concentration of a substance that results in a 50% effect on some measure of biochemical function or substance-target binding interaction.

A bicyclic ring system, as used herein, refers to heterocyclic (heterocyclyl) groups, to cyclic groups having carbon groups only, i.e. without hetero atoms, within the cycle, and to combinations of a heterocyclic (heterocyclyl) group and a cyclic group having carbon groups only, i.e. without hetero atoms, within the cycle.

A monocyclic ring system, as used herein, refers both to a heterocyclic (heterocyclyl) group, and to a cyclic group having carbon groups only, i.e. without hetero atoms, within the cycle.

A heterocyclic (heterocyclyl) group, as used herein, refers to both heteroaryl groups and heterocycloalkyl groups.

A heterobicyclic group, as used herein, refers to a bicyclic group having one or more heteroatoms, which is saturated, partially unsaturated or unsaturated.

As used herein, aromatic groups (or aryl groups) include aromatic carbocyclic ring systems (e.g. phenyl) and fused polycyclic aromatic ring systems (e.g. naphthyl and 1,2,3,4-tetrahydronaphthyl).

The term “alkyl,” as used herein, refers to an aliphatic hydrocarbon group having one of its hydrogen atoms replaced with a bond having the specified number of carbon atoms. In different embodiments, an alkyl group contains, for example, from 1 to 6 carbon atoms (1-6C)Alkyl or from 1 to 3 carbon atoms (1-3C)Alkyl. Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. In one embodiment, an alkyl group is linear. In another embodiment, an alkyl group is branched.

Unless specified otherwise, “alkyl” includes both branched- and straight-chain saturated aliphatic hydrocarbon groups, including all isomers, having the specified number of carbon atoms; for example, “(1-6C)Alkyl” includes all of the hexyl alkyl and pentyl alkyl isomers as well as n-, iso-, sec- and t-butyl, n- and isopropyl, ethyl and methyl. “Alkylene” refers to both branched- and straight-chain saturated aliphatic hydrocarbon groups, including all isomers, having the specified number of carbons, and having two terminal end chain attachments; for example, the term “A-C₄ alkylene-B” represents, for example, A—CH₂—CH₂—CH₂—CH₂—B, A—CH₂—CH₂—CH(CH₃)—CH₂—B, A—CH₂—CH(CH₂CH₃)—B, A—CH₂—C(CH₃)(CH₃)—B, and the like.

The term “alkylcarbonyl,” as used herein, refers to an aliphatic hydrocarbon group having one of its hydrogen atoms replaced with a bond attached to a carbonyl group, wherein the aliphatic hydrocarbon group has the specified number of carbon atoms. In different embodiments, an alkyl group or aliphatic hydrocarbon group contains, for example, from 1 to 6 carbon atoms (1-6C)Alkyl or from 1 to 3 carbon atoms (1-3C)Alkyl. Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. In one embodiment, an alkyl group is linear. In another embodiment, an alkyl group is branched.

Cycloalkyl means a cycloalkyl group having the recited number of carbon atoms, with the same meaning as previously defined, such as cyclopropyl, cyclobutyl, or cyclopentyl. “Cycloalkyl” refers to a cycloalkyl-group represented by an indicated number of carbon atoms; for example “(3-6C)cycloalkyl” includes cyclopropyl, cyclobutyl, cyclopentyl, or cyclohexyl.

Heterocycloalkyl means a cycloalkyl group having the recited number of carbon atoms, and 1-3 heteroatoms selected from N, O and/or S, with the same meaning as previously defined.

Haloalkyl means a branched or unbranched alkyl group having the recited number of carbon atoms, in which one and up to all hydrogen atoms are replaced by a halogen; halogen is as defined herein. Examples of such branched or straight chained haloalkyl groups useful in the present invention include, but are not limited to, methyl, ethyl, propyl, isopropyl, isobutyl and n-butyl substituted independently with one or more halogens, e.g., fluoro, chloro, bromo and iodo.

For example, a halo(1-3C)alkyl means a branched or unbranched alkyl group having 1,2, or 3 carbon atoms, in which at least one hydrogen atom is replaced by a halogen. Examples of “haloalkyl” include, but are not limited to, fluoromethyl, difluoromethyl, trifluoromethyl, 1-fluoroethyl, 2-fluoroethyl, 2,2-difluoroethyl, 2,2,2-trifluoroethyl, and perfluoro-n-propyl.

Alkoxy means an alkoxy group having the recited number of carbon atoms, the alkyl moiety having the same meaning as previously defined, e.g., “Alkoxy” refers to an alkyl-O-group represented by a linear or branched alkyl group of indicated number of carbon atoms attached through an oxygen bridge; for example “(1-6C)Alkoxy” includes—OCH₃, —O—CH₂CH₃, —OCH(CH₃)₂, —O(CH₂)₅CH₃, and the like.

Cycloalkoxy means a cycloalkyl group having the recited number of carbon atoms, with the same meaning as previously defined, attached via a ring carbon atom to an exocyclic oxygen atom, such as cyclopropoxyl, cyclobutoxyl, or cyclopentoxyl. “Cycloalkoxy” refers to a cycloalkyl-O-group represented by a cycloalkyl group of indicated number of carbon atoms attached through an oxygen bridge; for example “(3-6C)cycloalkoxy” includes cyclopropyl-O-, cyclobutyl-O-, cyclopentyl-O-, or cyclohexyl-O-.

Heterocycloalkoxy means a cycloalkyl group having the recited number of carbon atoms, and 1-3 heteroatoms selected from N, O and/or S, with the same meaning as previously defined, attached via a ring carbon atom to an exocyclic oxygen atom.

Unless otherwise specifically noted as only “unsubstituted” or only “substituted”, alkyl groups are unsubstituted or substituted with 1 to 3 substituents on each carbon atom.

It should be noted that any carbon as well as heteroatom with unsatisfied valences in the text, schemes, examples and tables herein is assumed to have the sufficient number of hydrogen atom(s) to satisfy the valences.

The terms first, second, third and the like in the description and in the claims, are used for distinguishing between for example similar elements, compositions, constituents in a composition, or separate method steps, and not necessarily for describing a sequential or chronological order. The terms are interchangeable under appropriate circumstances and the embodiments of the invention can operate in other sequences than described or illustrated herein, unless specified otherwise.

Furthermore, the various embodiments, although referred to as “preferred” or “e.g.” or “for example” or “in particular” and the like are to be construed as exemplary manners in which the invention may be implemented rather than as limiting the scope of the invention.

The term “comprising”, used in the claims, should not be interpreted as being restricted to for example the elements or the method steps or the constituents of a compositions listed thereafter; it does not exclude other elements or method steps or constituents in a certain composition. It needs to be interpreted as specifying the presence of the stated features, integers, (method) steps or components as referred to, but does not preclude the presence or addition of one or more other features, integers, steps or components, or groups thereof. Thus, the scope of the expression “a method comprising steps A and B” should not be limited to a method consisting only of steps A and B, rather with respect to the present invention, the only enumerated steps of the method are A and B, and further the claim should be interpreted as including equivalents of those method steps. Thus, the scope of the expression “a composition comprising components A and B” should not be limited to a composition consisting only of components A and B, rather with respect to the present invention, the only enumerated components of the composition are A and B, and further the claim should be interpreted as including equivalents of those components.

In addition, reference to an element or a component by the indefinite article “a” or “an” does not exclude the possibility that more than one of the element or component are present, unless the context clearly requires that there is one and only one of the elements or components.

The indefinite article “a” or “an” thus usually means “at least one”.

### LEGEND TO THE FIGURES

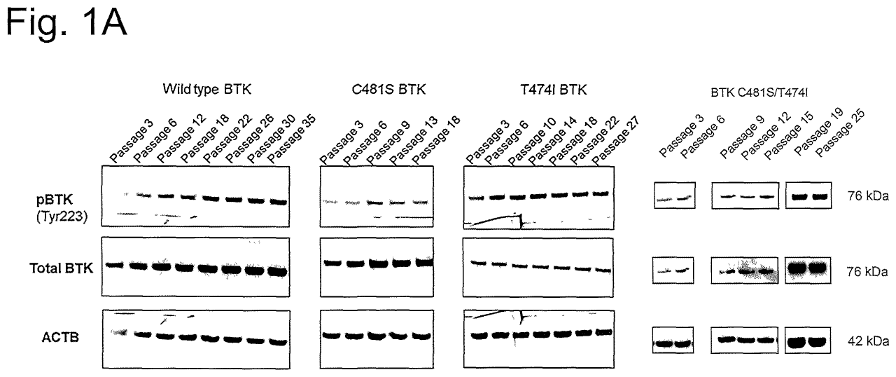

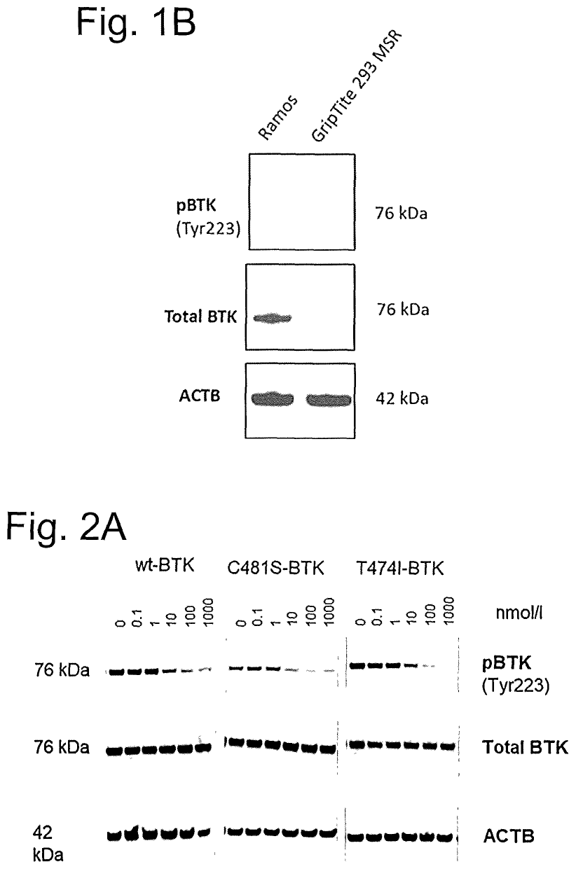

FIG. 1:
(A) Stability of (mutant) BTK and phospho-(Tyr223)BTK protein expression in wild-type BTK or in mutant BTK (BTK C481S; BTK T474I; BTK C481 S/T474I) transfected 293 cells cultured in DMEM/F-12, GlutaMAX™ supplement with 1% Penicilline/Streptomycine, 10% fetal bovine serum, 1× MEM Non-Essential Amino Acids, 50 μg/ml Geneticin and Blasticidin.
(B) BTK and phospho-BTK protein expression in wildtype 293 (GripTite 293 MSR) cells compared to (non-IgM treated) RAMOS cells.

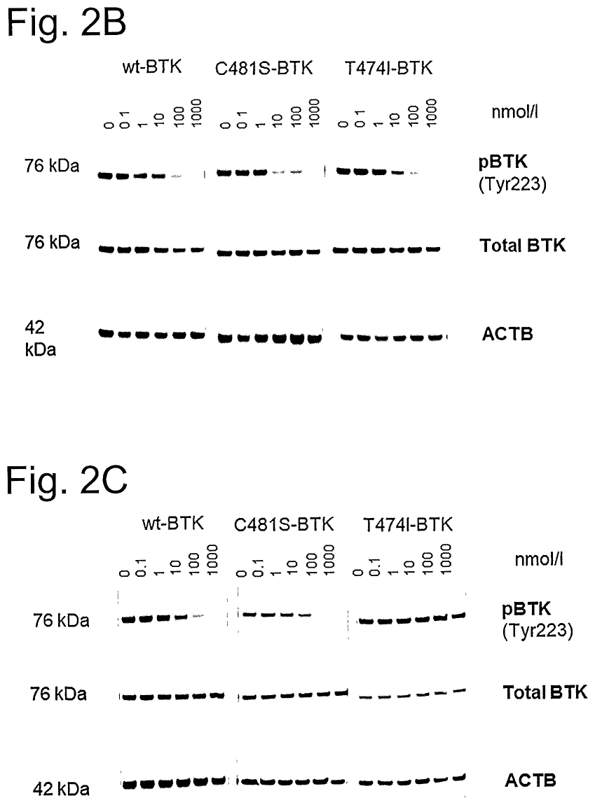

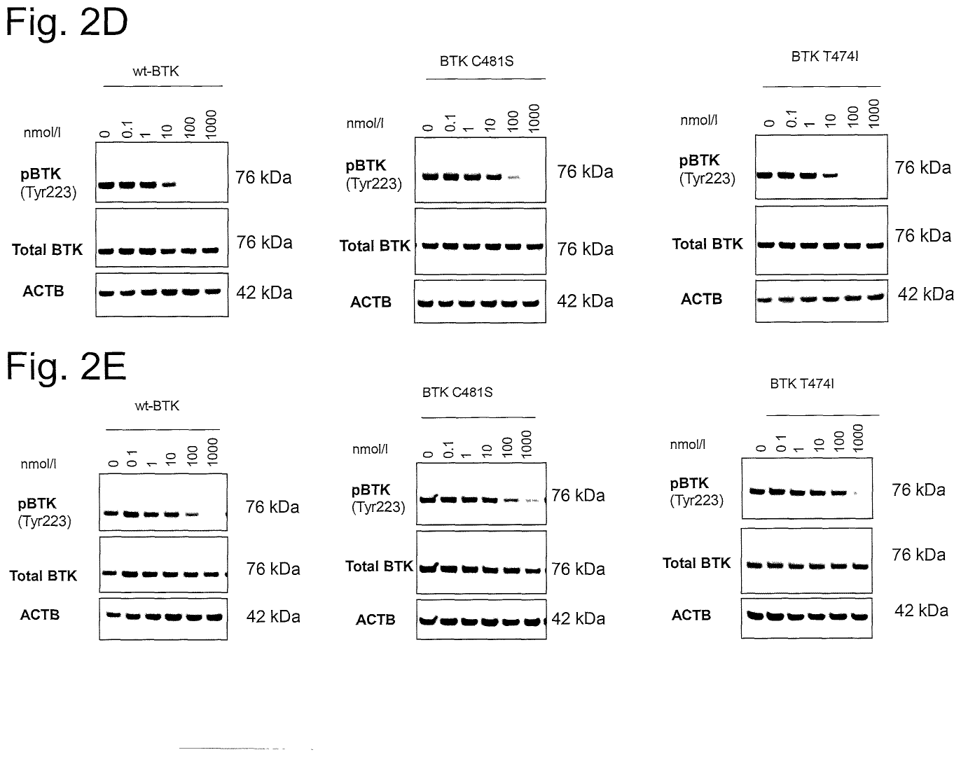

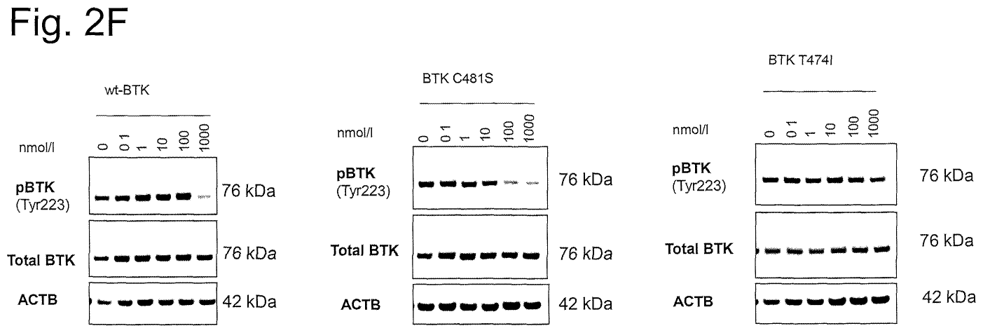

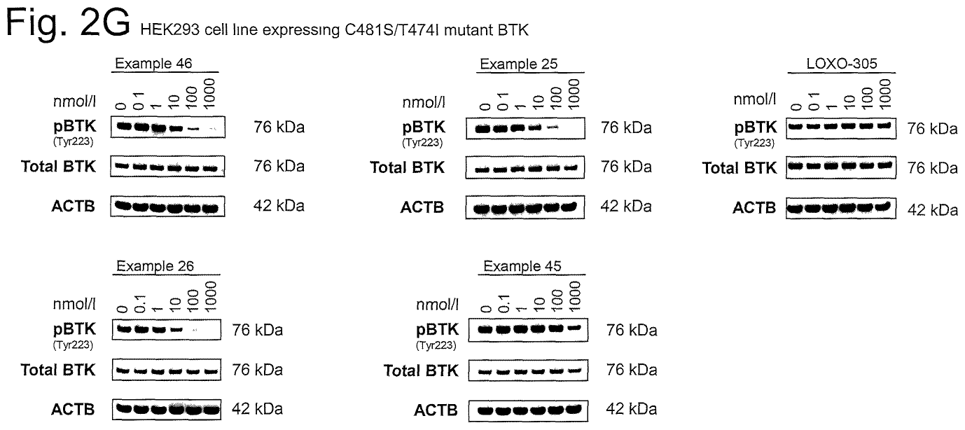

FIG. 2:
(A) Western blot protein expression results of wt-BTK, BTK C481S, and BTK T474I expressing 293 cells incubated with 0, 0.1, 1.0, 10, 100 and 1000 nM of Example 46;
(B) Western blot protein expression results of wt-BTK, BTK C481S, and BTK T474I expressing 293 cells incubated with 0, 0.1, 1.0, 10, 100 and 1000 nM of Example 25;
(C) Western blot protein expression results of wt-BTK, BTK C481S, and BTK T474I expressing 293 cells incubated with 0, 0.1, 1.0, 10, 100 and 1000 nM (+/−) Loxo-305;
(D) Western blot protein expression results of wt-BTK, BTK C481S, and BTK T474I expressing 293 cells incubated with 0, 0.1, 1.0, 10, 100 and 1000 nM of Example 26;
(E) Western blot protein expression results of wt-BTK, BTK C481S, and BTK T474I expressing 293 cells incubated with 0, 0.1, 1.0, 10, 100 and 1000 nM of Example 83;
(F) Western blot protein expression results of wt-BTK, BTK C481 S, and BTK T474I expressing 293 cells incubated with 0, 0.1, 1.0, 10, 100 and 1000 nM of Example 68;
(G) Western blot protein expression results of double mutant BTK C481 S/T474I expressing 293 cells incubated with 0, 0.1, 1.0, 10, 100 and 1000 nM of Example 25, Example 26, Example 45, Example 46, and Loxo-305;

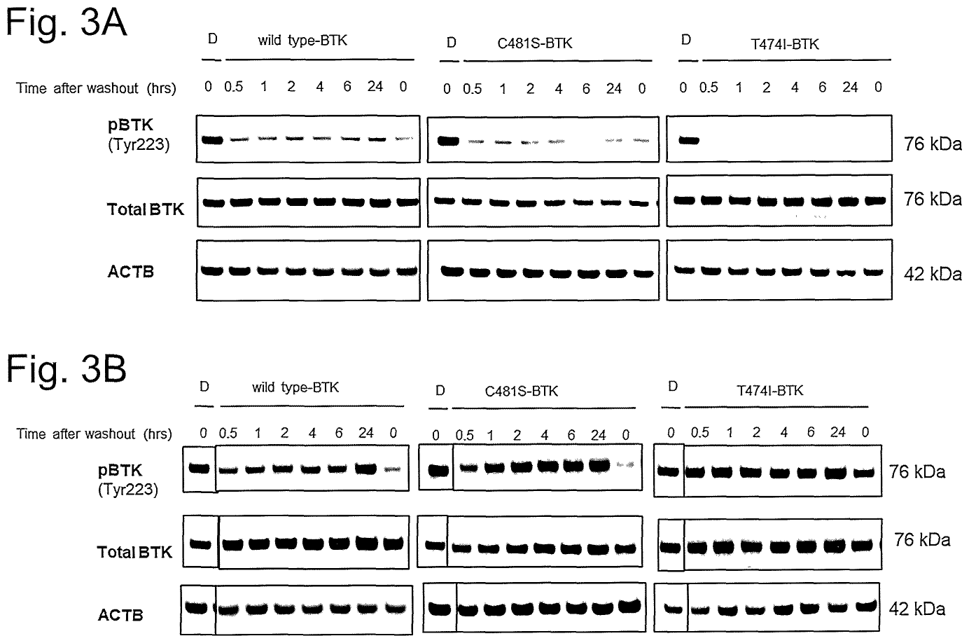

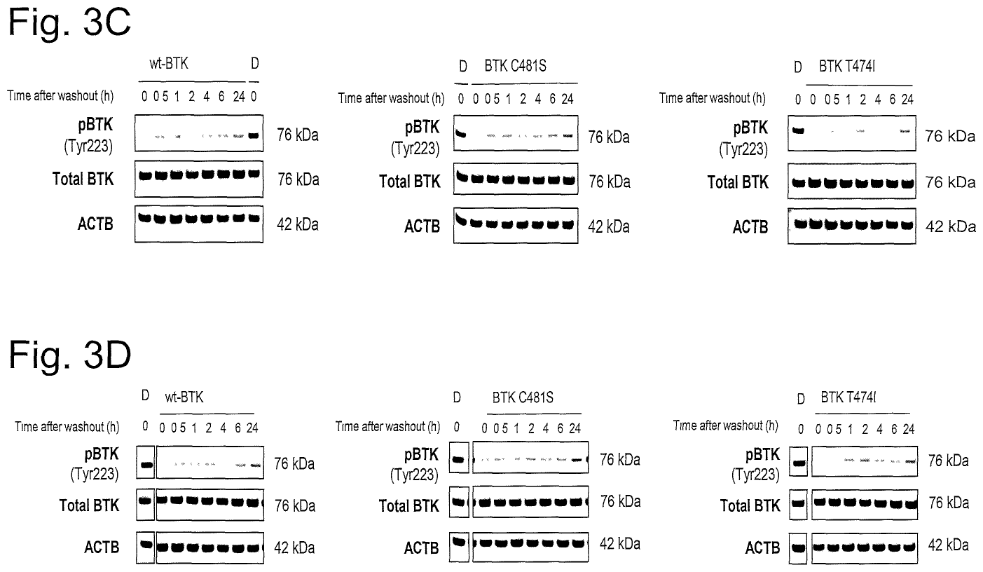

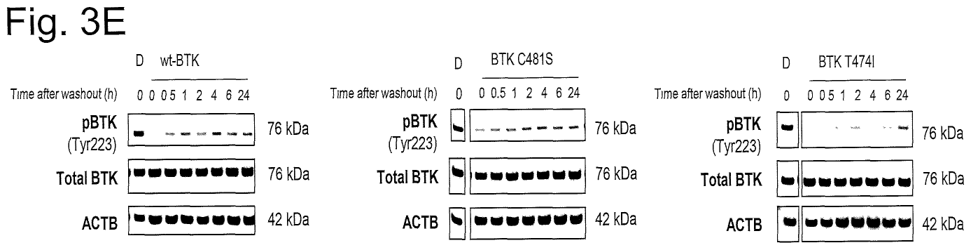

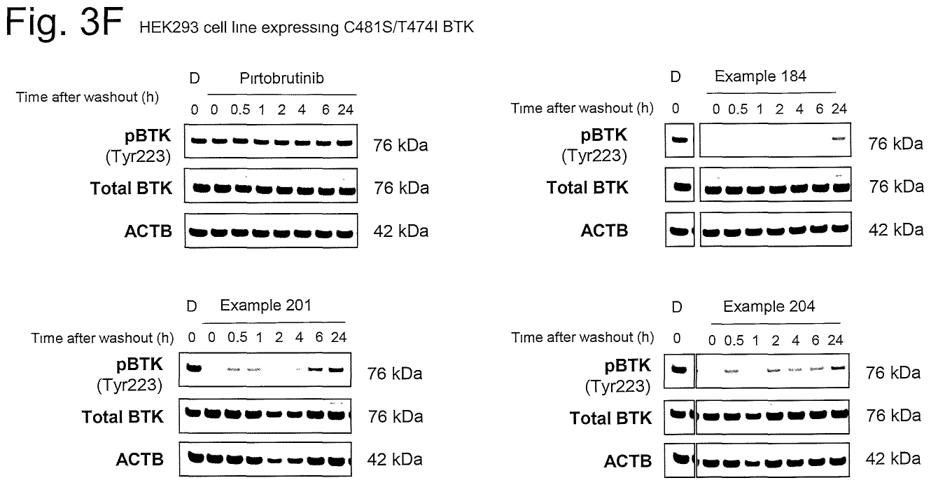

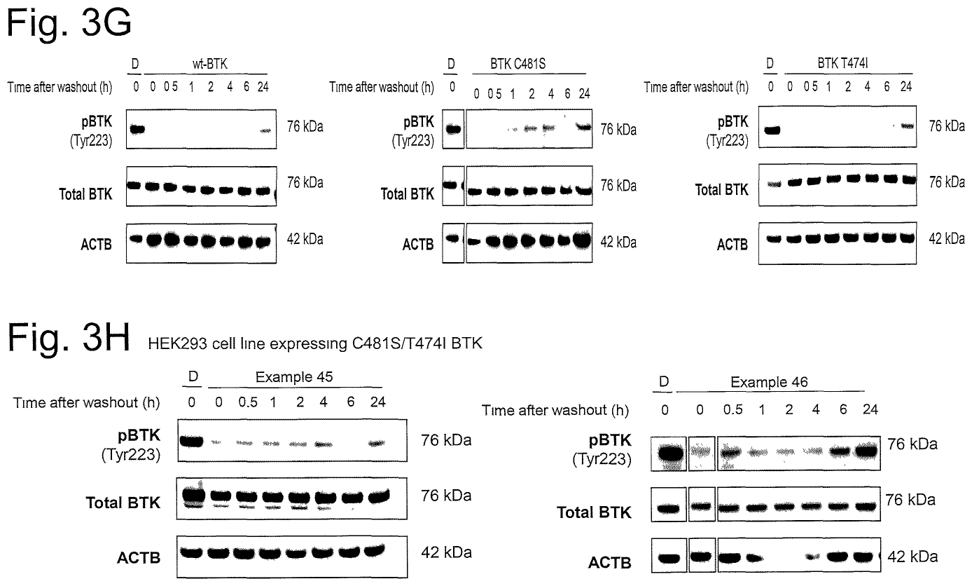

FIG. 3:
Wash-out Western blot results of wt-BTK, BTK C481 S, BTK T474I and BTK C481 S/T474I expressing 293 cells. Cells were incubated with 1000 nM of compound of Example 46 or 1000 nM of compound of Example 184 or 1000 nM of compound of Example 201 or 1000 nM of compound of Example 204 or 1000 nM of compound (+/−) Loxo-305 in cell culture medium without blasticidin. Compound containing medium was removed after 2h hours and cells were washed twice before being replaced by medium without compound. Cells were harvested after 0, 0.5, 1, 2, 4, 6 and 24 hours after medium replacement. Control cells (no compound) were incubated for two hours with 0.01% DMSO (D) and harvested at 0 hours (0h), which is at the start of the wash-out, and 24 hours (24h). The level of phosphorylated BTK (pBTK (Tyr223)) was determined on Western blot for compound of Example 46 as shown in FIG. 3A, for compound (+/−) Loxo-305 as shown in FIG. 3B, for compound of Example 201 as shown in FIG. 3C, for compound of Example 184 as shown in FIG. 3D and for compound of Example 204 as shown in FIG. 3E. FIG. 3F shows Western blot protein expression results of wash-outs of double mutant BTK C481 S/T474I expressing 293 cells incubated with 1000 nM of compound of Example 184 or 1000 nM of compound of Example 201 or 1000 nM of compound of Example 204 or 1000 nM of compound of pirtobrutinib (loxo-305). The level of phosphorylated BTK (pBTK (Tyr223)) was determined on Western blot for compound of Example 45 as shown in FIG. 3G for wt-BTK, BTK C481 S and BTK T474I expressing 293 cells. The level of phosphorylated BTK (pBTK (Tyr223)) was determined on Western blot for compound of Example 45 and of compound of Example 46 as shown in FIG. 3H for BTK C481 S/T474I expressing 293 cells. Beta-actin (ACTB) and BTK (total BTK) levels were determined as loading control and BTK expression control.

### DETAILED DESCRIPTION OF THE INVENTION

Surprisingly, the inventors have found that compounds according to the invention provide an improved reversible binding activity towards wild-type BTK and/or BTK mutants. The compounds according to the invention have any one of Formula (I-a) to (I-h), which contains a macrocyclic moiety, in combination with BTK specific pharmacophores (e.g. based on ligands for binding to BTK) to provide a binding activity towards wild-type BTK and/or BTK mutants through improved reversible binding. In particular it has been found that the compound provides a considerable longer residence time than what is typically obtained with reversible BTK inhibitors.

The inventors have established that the reversible inhibitors pirtobrutinib, vecabrutinib and fenebrutinib all showed short target residence times on wild-type-BTK as well as on BTK C481, T316 or T474 mutants.

Additionally, the inventors have surprisingly found that macrocyclic compounds of Formula I-a to I-h provide an enhanced binding activity towards BTK mutant forms. In exemplary embodiments the inventors have demonstrated the enhanced binding activity towards BTK mutants BTK C481S, BTK T316A, BTK T474I and BTK T474S. Based on these findings, and based on the macrocyclic moiety effect on the (BTK targeted) compounds of the invention, it is anticipated that binding activity to other BTK mutants is also enhanced.

Macrocyclic natural products have advanced to achieve numerous biochemical functions, and their pharmacological properties have led to their development as drugs. Macrocycles have been defined as a ring system consisting of 12 or more atoms (Driggers E. M. (2008) Nat Rev Drug Discov). A macrocycle provides diverse functionalities and stereochemical complexity in a conformationally pre-organized ring structure, which can result in superb physicochemical and pharmacological properties. By limiting the number of (bioactive) conformations available to the unbound molecule, it is believed that there is a lower entropic cost when the molecule interacts with its target protein as compared to a non-macrocyclic compound. Macrocyclic ligands may be selected to displace ordered water molecules from a binding site unoccupied by non-macrocyclic inhibitors into bulk solvent. This may be assumed to provide a second favorable entropic contribution (classical hydrophobic effect) (Mallinson J. M. and Collins I. (2012) Future Med Chem). Leading to enhanced potencies of these inhibitors on their target protein.

Now, the inventors have found that compounds according to the invention, which comprise a macrocyclic moiety in addition to active binding parts, provide an improved binding activity towards BTK and/or BTK mutants, compared to similar compounds, which provide a binding activity, but do not contain a macrocycle.

### Embodiments

### Compounds of the Invention

Compounds of the invention are according to Formula (1-a) to (1-h):

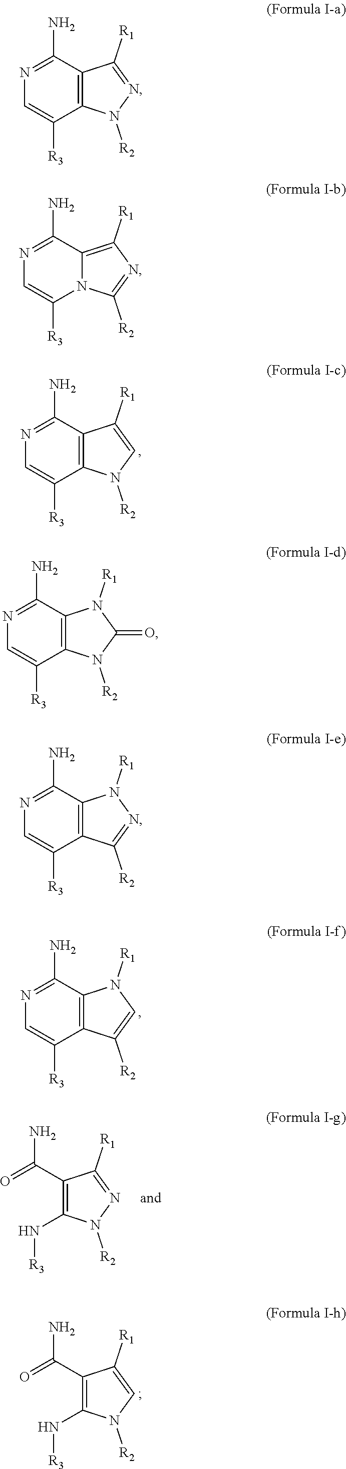

wherein said compound as further described below, or a pharmaceutically acceptable salt and/or solvate thereof.

In a first aspect of the invention is provided a compound of Formula (I-a) to (I-h) or a pharmaceutically acceptable salt and/or solvate thereof, wherein the compound is selected from the group consisting of:

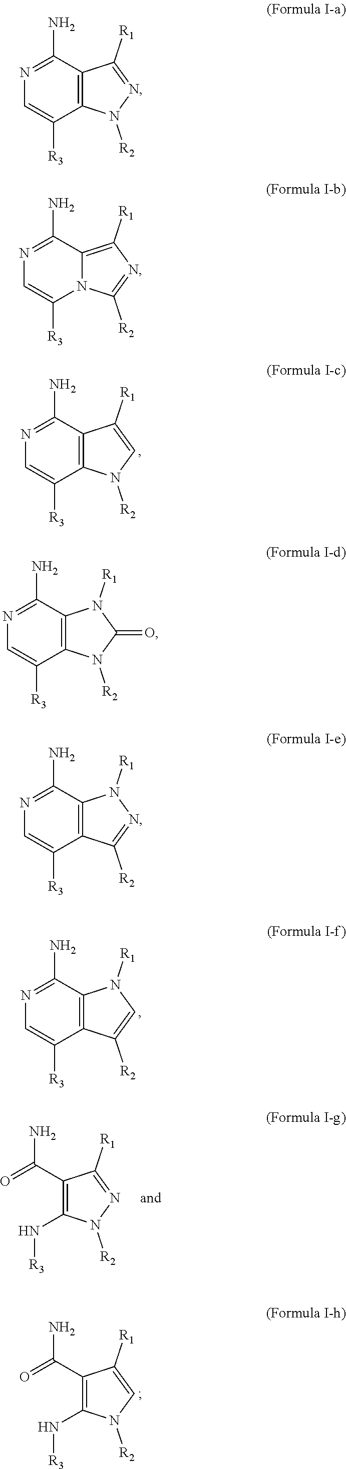

Wherein R¹ is

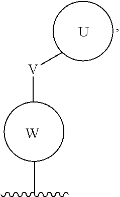

wherein:

W is an aryl group having 6-10 carbon or a heteroaryl group having 1-5 carbon; wherein any said aryl group and heteroaryl group is optionally and independently substituted with one or more substituents selected from halogen, (1-2C)alkyl, (1-2C)alkoxy; wherein any of said alkyl and alkoxy group is optionally and independently substituted with one, two or three fluoro; V is any one of O, —C(O)—NH—, —NH—C(O)—, —CH(R^{1v})—NH—C(O)—, —CH(R^{1v})—; R^{1v} is hydrogen or (1-2C)alkyl;

U is an aryl group having 6-10 carbon or an heteroaryl group having 1-5 carbon; wherein any of said aryl group and heteroaryl group is optionally and independently substituted with one or more substituents selected from halogen, cyano, (1-4C)alkyl, (1-5C)alkoxy, (3-6C)cycloalkyl or (3-6C)heterocycloalkyl; wherein any of said alkyl, alkoxy, cycloalkyl and heterocycloalkyl group is optionally and independently substituted with one, two or three halogen; R² is of Formula (II-a) to (II-f) selected from the group consisting of:

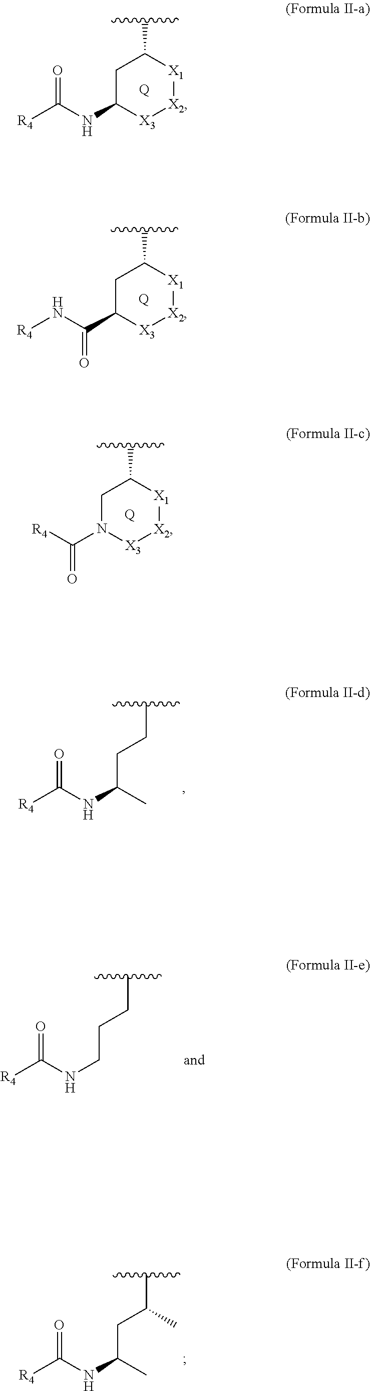

wherein Q is a monocyclic ring selected from a (3-7C)cycloalkyl and a (3-6C)heterocycloalkyl, wherein X₁, X₂ and X₃ are independently selected from CH₂, —CH₂CH₂—, O, N and a direct bond; wherein any of the cycloalkyl, heterocycloalkyl and alkyl group is optionally and independently substituted with one or more substituents selected from halogen, hydroxy, (1-3C)alkyl, (1-3C)alkoxy, (1-4C)alkylcarbonyl or (3-4C)cycloalkyl; wherein any of said alkyl and alkoxy group is optionally and independently substituted with one, two or three halogen;

R³ and R⁴ together represent a linker having Formula (III-1) to (III-40) selected from the group consisting of:

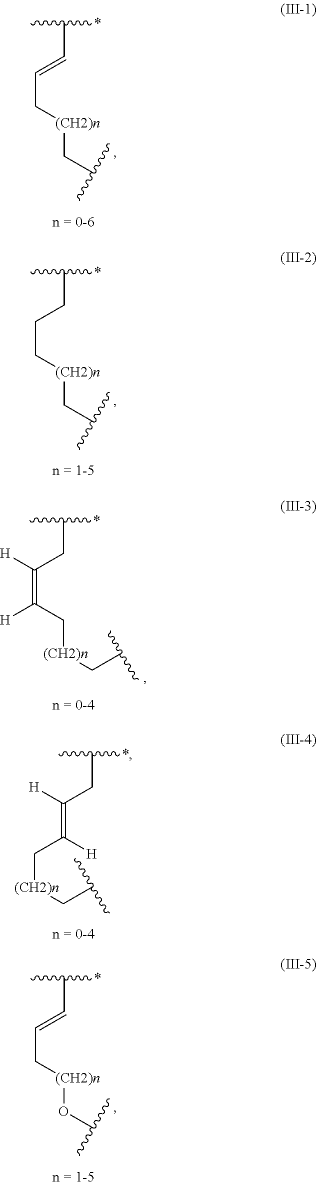

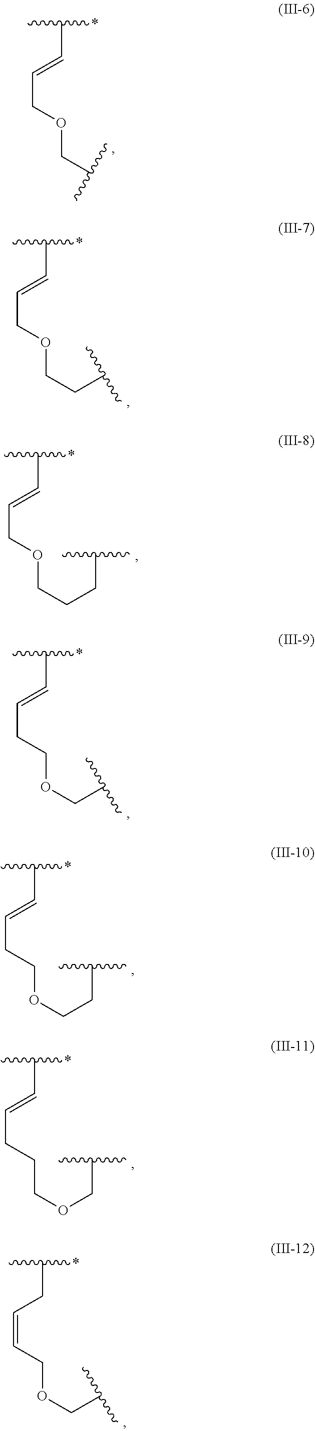

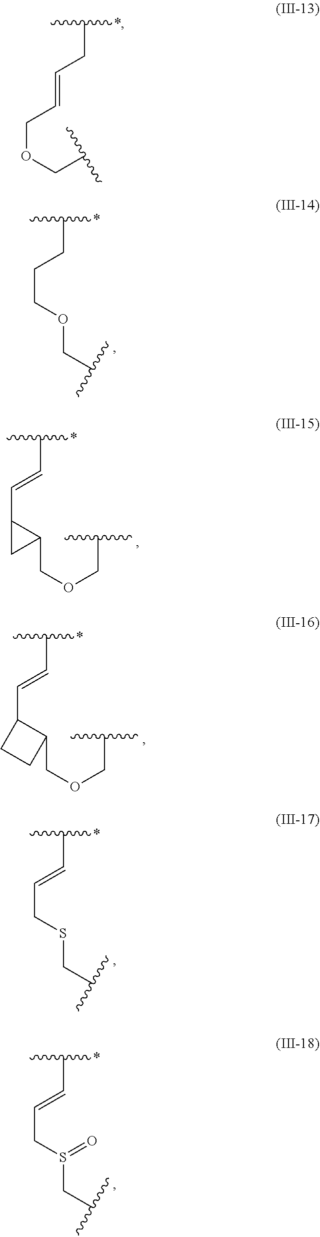

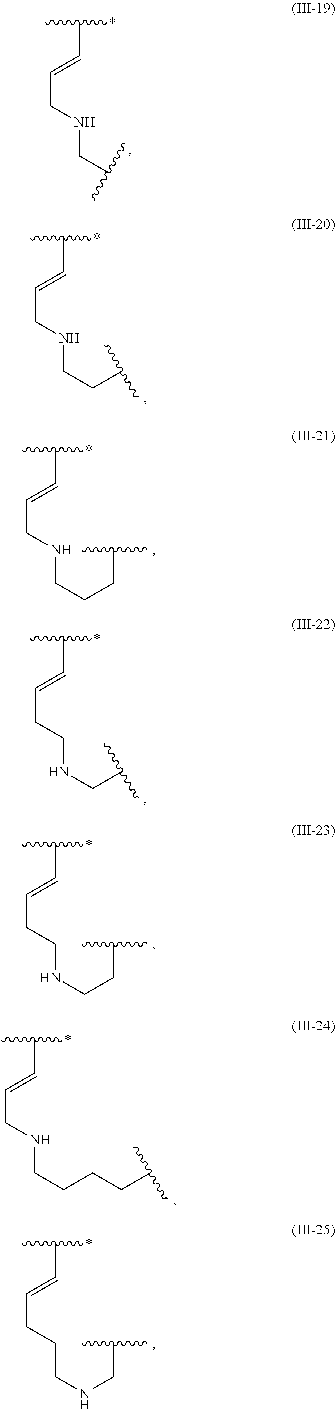

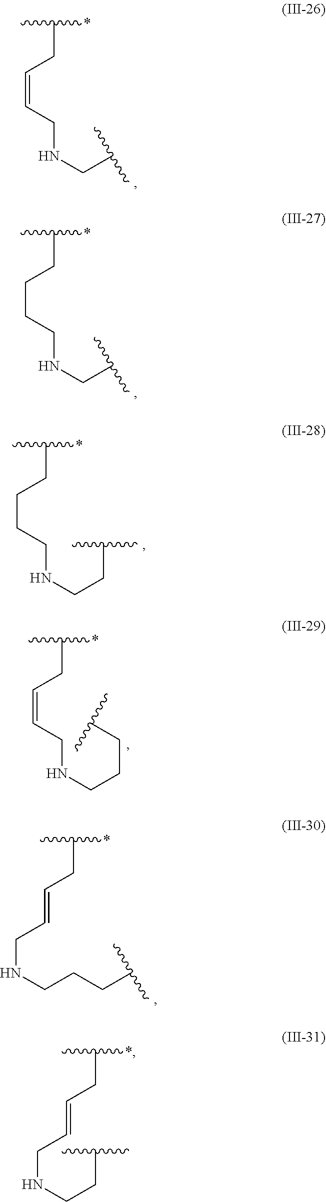

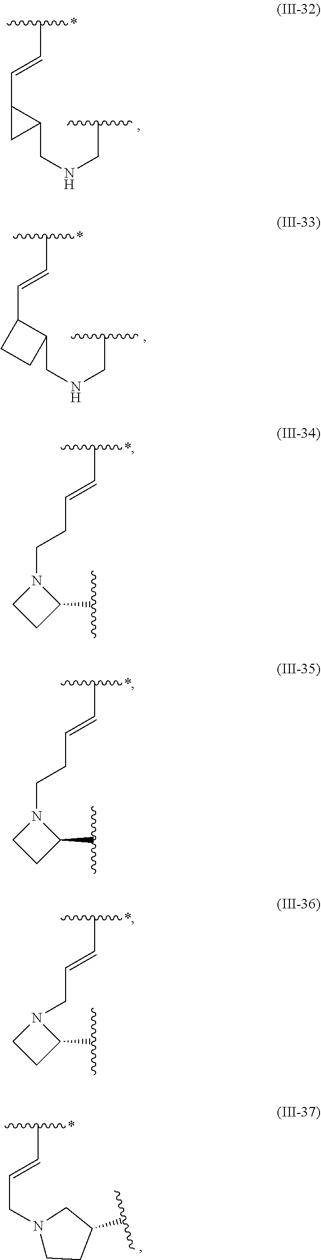

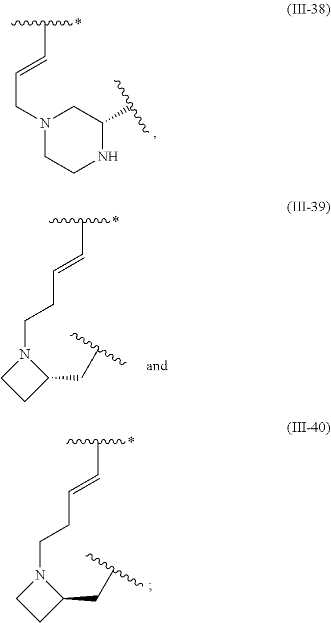

whereby the

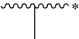

marks the position of R³ in any one of Formula I-a to I-h, and whereby the

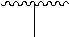

marks the position of R⁴ in any one of Formula II-a to II-f;
wherein any of said linkers is optionally and independently substituted with one or more substituents selected from deuterium, halogen, oxo, hydroxy, CD₃, (1-4C)alkyl, (1-5C)alkoxy, (3-6C)cycloalkyl, (3-6C)cycloalkoxy and (1-6C)alkylcarbonyl; wherein any of said alkyl and alkoxy group is optionally and independently substituted with one, two or three halogen.

In preferred embodiments, the present invention provides compounds of sub-formulas 1-226 as described below. The values of substituents in preferred groups of compounds of sub-formulas 1-226 are given below.

### (R¹)

In an aspect of the invention the R¹ of the compounds of the invention has the formula:

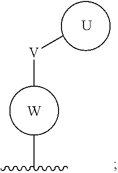

wherein:

W is an aryl group having 6-10 carbon or a heteroaryl group having 1-5 carbon; wherein any said aryl group and heteroaryl group is optionally and independently substituted with one or more substituents selected from halogen, (1-2C)alkyl, (1-2C)alkoxy; wherein any of said alkyl and alkoxy group is optionally and independently substituted with one, two or three fluoro;

V is any one of O, —C(O)—NH—, —NH—C(O)—, —CH(R^{1v})—NH—C(O)—, —CH(R^{1v})—;

R^{1v} is hydrogen or (1-2C)alkyl;

and U is an aryl group having 6-10 carbon or an heteroaryl group having 1-5 carbon; wherein any of said aryl group and heteroaryl group is optionally and independently substituted with one or more substituents selected from halogen, cyano, (1-4C)alkyl, (1-5C)alkoxy, (3-6C)cycloalkyl or (3-6C)heterocycloalkyl; wherein any of said alkyl, alkoxy, cycloalkyl and heterocycloalkyl group is optionally and independently substituted with one, two or three halogen.

In embodiments, wherein R¹ is selected from the group consisting of:

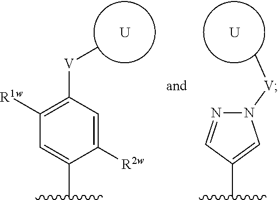

wherein R^{1w} and R^{2w} are independently selected from hydrogen, halogen, (1-2C)alkyl, (1-2C)alkoxy; wherein any of said alkyl and alkoxy group is optionally and independently substituted with one, two or three fluoro;

V is any one of O, —C(O)—NH—, —NH—C(O)—, —CH(R^{1v})—NH—C(O)—, —CH(R^{1v})—;

R^{1v} is hydrogen or (1-2C)alkyl;

U is an aryl group having 6-10 carbon or an heteroaryl group having 1-5 carbon; wherein any of said aryl group and heteroaryl group is optionally and independently substituted with one or more substituents selected from halogen, cyano, (1-4C)alkyl, (1-5C)alkoxy, (3-6C)cycloalkyl or (3-6C)heterocycloalkyl; wherein any of said alkyl, alkoxy, cycloalkyl and heterocycloalkyl group is optionally and independently substituted with one, two or three halogen.

In embodiments, wherein R¹ is any of:

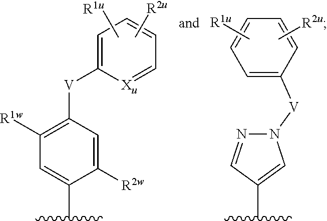

wherein R^{1w} and R^{2w} are independently selected from hydrogen, halogen, (1-2C)alkyl, (1-2C)alkoxy; wherein any of said alkyl and alkoxy group is optionally and independently substituted with one, two or three fluoro;

V is any one of O, —C(O)—NH—, —NH—C(O)—, —CH(R^{1v})—NH—C(O)—, —CH(R^{1v})—;

R^{1v} is hydrogen or (1-2C)alkyl;

wherein R^{1u} and R^{2u} are independently selected from hydrogen, halogen, cyano, (1-4C)alkyl, (1-5C)alkoxy, (3-6C)cycloalkyl or (3-6C)heterocycloalkyl; wherein any of said alkyl and alkoxy group is optionally and independently substituted with one, two or three halogen; wherein X^{U} is selected from CH and N.

In embodiments, wherein V is any one of O, —C(O)—NH—, —CH(R^{1v})—NH—C(O)—, —CH(R^{1v})-; R^{1v} is hydrogen or (1-2C)alkyl.

In embodiments, wherein R¹ is any one of:

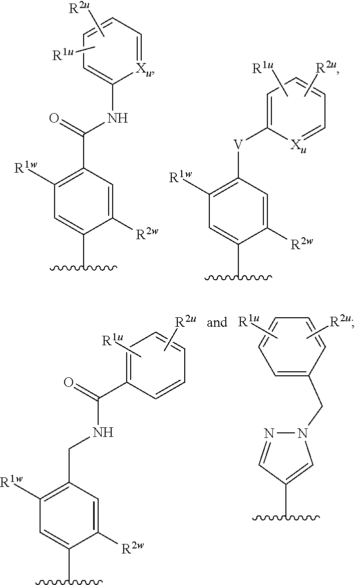

wherein R^{1w} and R^{2w} are independently selected from hydrogen, halogen, (1-2C)alkyl, (1-2C)alkoxy; wherein any of said alkyl or alkoxy group is optionally and independently substituted with one, two or three fluoro;

wherein R^{1u} and R^{2u} are independently selected from hydrogen, halogen, cyano, (1-4C)alkyl, (1-5C)alkoxy, (3-6C)cycloalkyl or (3-6C)heterocycloalkyl; wherein any of said alkyl and alkoxy group is optionally and independently substituted with one, two or three halogen; wherein X^{u} is selected from CH and N.

In embodiments, wherein R¹ is any one of:

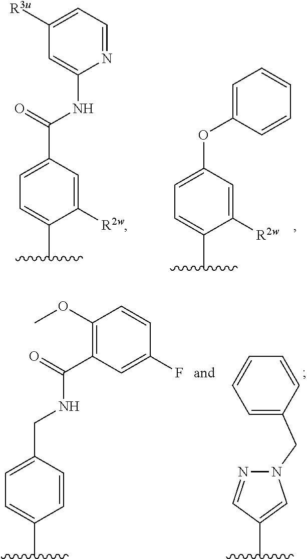

wherein R^{2w} is selected from hydrogen, halogen, (1-2C)alkyl, (1-2C)alkoxy; wherein any said alkyl or alkoxy group is optionally and independently substituted with one, two or three fluoro; wherein R^{3u} is selected from hydrogen, halogen, cyano, (1-4C)alkyl, (1-5C)alkoxy, (3-6C)cycloalkyl or (3-6C)heterocycloalkyl; wherein any of said alkyl and alkoxy group is optionally and independently substituted with one, two or three fluoro.

In embodiments, wherein R¹ is any one of:

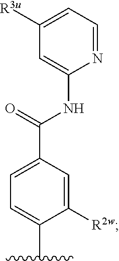

wherein R^{2w} is selected from hydrogen, fluoro, methyl or methoxy;

wherein R^{3u} is selected from hydrogen, halogen, cyano, (1-4C)alkyl, (1-2C)alkoxy, (3-6C)cycloalkyl or (3-6C)heterocycloalkyl; wherein any of said alkyl and alkoxy group is optionally and independently substituted with one, two or three fluoro.

### (R²)

In an aspect of the invention the R² of the compounds of the invention is of Formula (II-a) to (II-f) selected from the group consisting of:

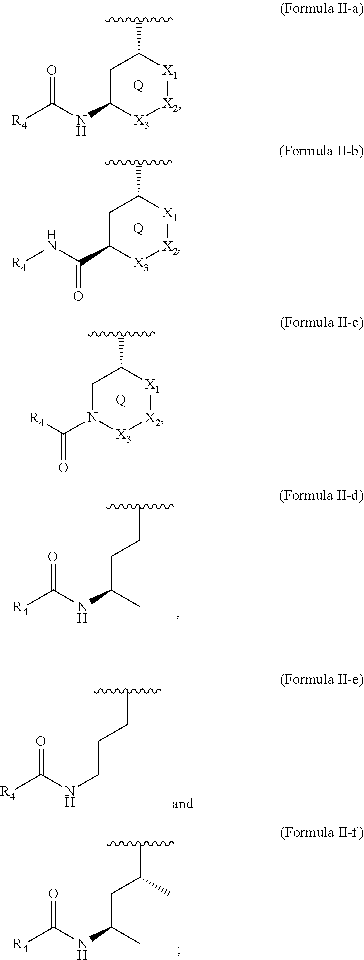

wherein Q is a monocyclic ring selected from a (3-7C)cycloalkyl and a (3-6C)heterocycloalkyl, wherein X₁, X₂ and X₃ are independently selected from CH₂, —CH₂CH₂—, O, N and a direct bond.

In embodiments, any of the cycloalkyl, heterocycloalkyl and alkyl group is optionally and independently substituted with one or more substituents selected from halogen, hydroxy, (1-3C)alkyl, (1-3C)alkoxy, (1-4C)alkylcarbonyl or (3-4C)cycloalkyl. In particular embodiment, any of said alkyl and alkoxy group is optionally and independently substituted with one, two or three halogen.

In embodiments, wherein R² is selected from the group consisting of:

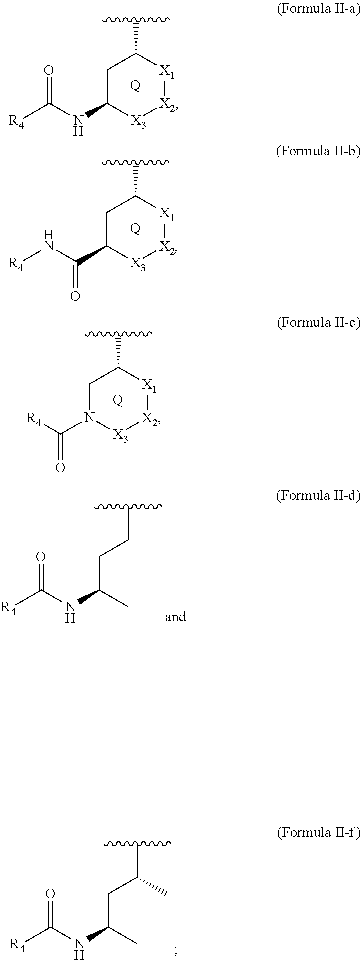

wherein Q is a monocyclic ring selected from a (3-7C)cycloalkyl and a (3-6C)heterocycloalkyl, wherein X₁, X₂ and X₃ are independently selected from CH₂, CH₂CH₂, O, N and a direct bond; wherein any of the cycloalkyl, heterocycloalkyl and alkyl group is optionally and independently substituted with one or more substituents selected from halogen, hydroxy, (1-3C)alkyl, (1-3C)alkoxy, (1-4C)alkylcarbonyl or (3-4C)cycloalkyl; wherein any of said alkyl and alkoxy group is optionally and independently substituted with one, two or three halogen.

In preferred embodiments, wherein R² is selected from the group consisting of:

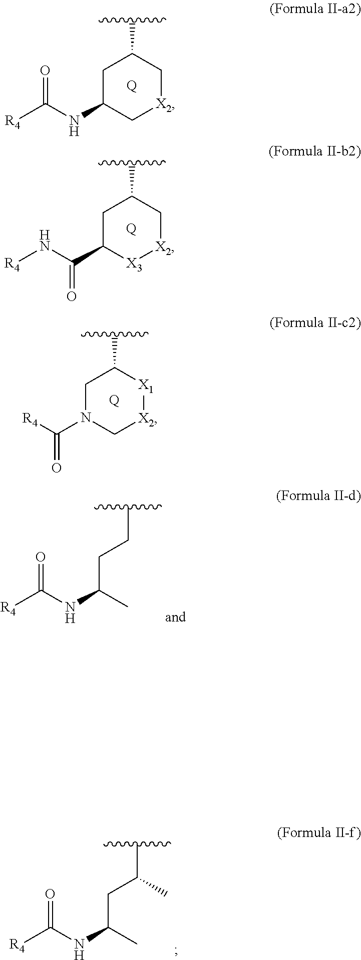

wherein Q is a monocyclic ring selected from a (3-7)cycloalkyl and a (3-6C)heterocycloalkyl, wherein X₁, X₂ and X₃ are independently selected from CH₂, O, N and a direct bond; wherein any of the cycloalkyl, heterocycloalkyl and alkyl group is optionally and independently substituted with one or more substituents selected from halogen, hydroxy, (1-3C)alkyl, (1-3C)alkoxy, (1-4C)alkylcarbonyl or (3-4C)cycloalkyl; wherein any of said alkyl and alkoxy group is optionally and independently substituted with one, two or three halogen.

In preferred embodiments, wherein R² is selected from the group consisting of:

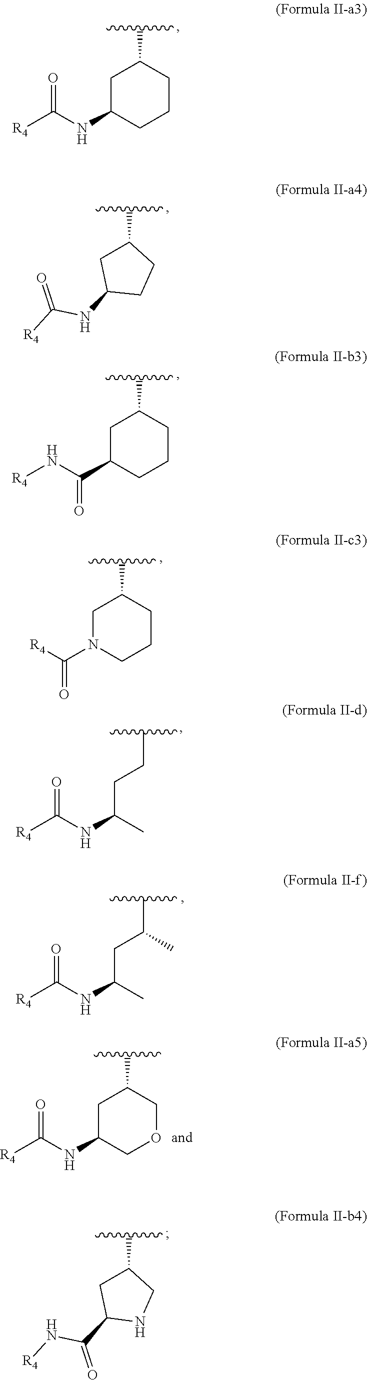

wherein any of said cycloalkyl, heterocycloalkyl and alkyl group is optionally and independently substituted with hydroxy, methyl, acetyl or methoxy.

In preferred embodiments, at least one of the X₁, X₂ and X₃ of R² is a nitrogen atom forming a secondary amine group, wherein the amine group is substituted by (1-4C)alkylcarbonyl, preferably by methylcarbonyl or ethylcarbonyl. In a preferred example the secondary amine group of Formula (II-b4)—of the monocyclic ring may be substituted by (1-4C)alkylcarbonyl, such as by methylcarbonyl or ethylcarbonyl.

(Linker Represented by R³ and R⁴)

Each of the compounds of the invention comprises the linker represented by R³ and R⁴. The linker is a part of the macrocycle of each of the compounds of the invention. In the given Formulas of the linkers having Formula (III-1) to (III-40), the

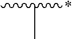

(i.e. the wavy with a star) marks the position of R³ in any one of Formula I-a to I-h, and the

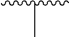

(i.e. the wavy without a star) marks the position of R⁴ in any one of Formula II-a to II-f. Thus, the linker is directly connected to the scaffold of any one of Formula I-a to I-h at the position of the

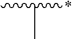

(i.e. the wavy with a star).

The macrocycle is formed by the connections between the linker, R² and the scaffold of Formula (I-a) to (I-h). R² is directly connected to the linker at the position of R⁴. The scaffold of Formula (I-a) to (I-h) is connected to the linker, at another end of the linker, at the position of R³. The scaffold is a bicycle shown in the compounds of Formula (I-a) to (I-f), or the scaffold is a monocycle shown in the compounds of Formula (I-g) to (I-h).

In embodiments, the macrocycle may comprise at least 12 atoms forming said macrocycle, and may comprise any number of atoms from 12-18 forming said macrocycle, preferably from 13-15 atoms forming said macrocycle.

In an embodiment, wherein the linker represented by R³ and R⁴ is selected from the group consisting of:

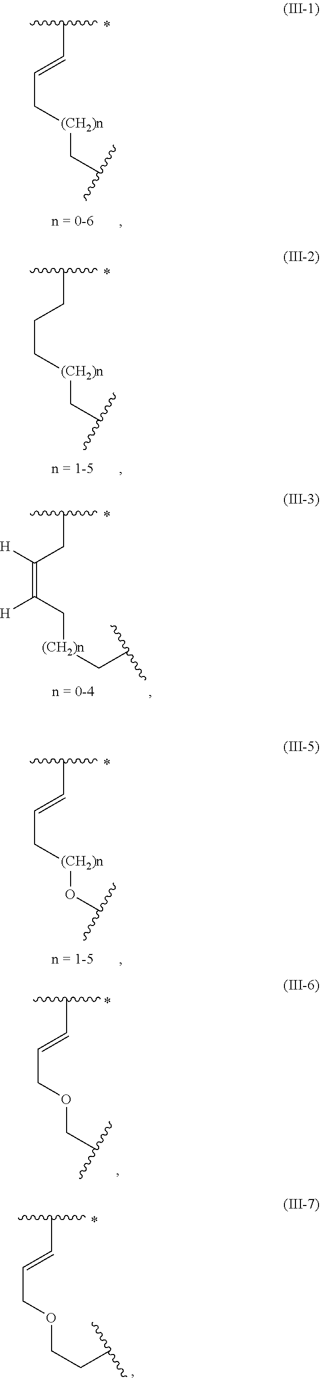

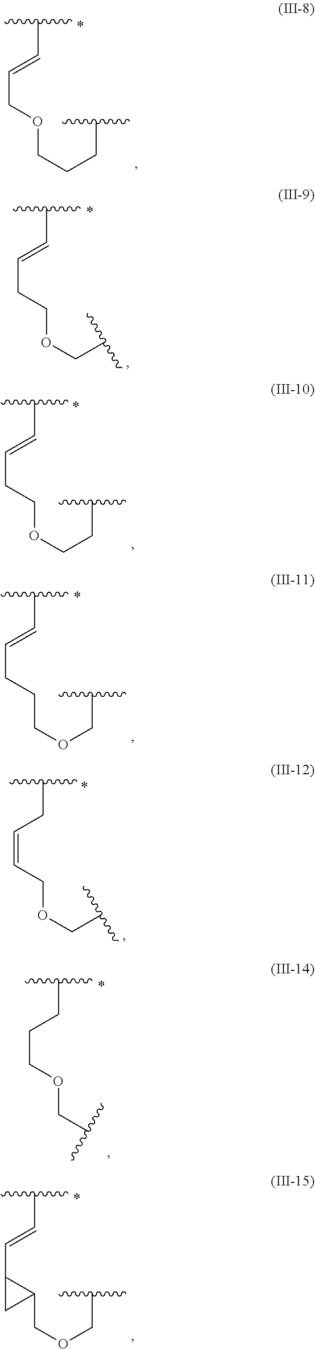

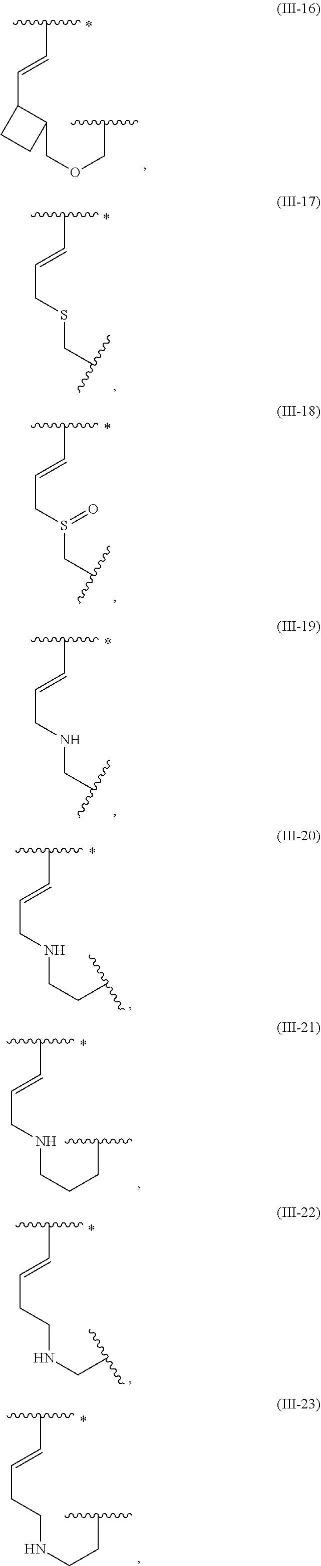

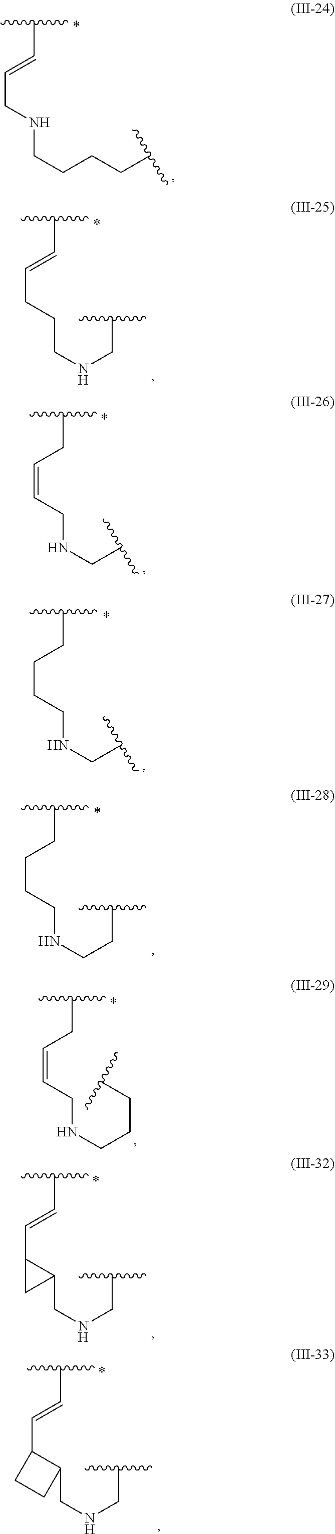

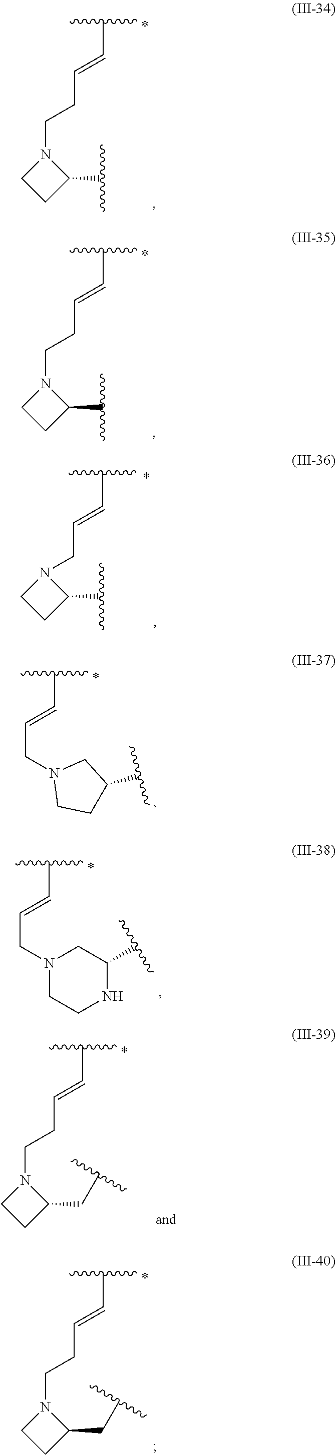

whereby the

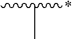

marks the position of R³ in any one of Formula I-a to I-h, and whereby the

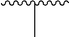

marks the position of R⁴ in any one of Formula II-a to II-f; wherein any of said linkers is optionally and independently substituted with one or more substituents selected from deuterium, halogen, oxo, hydroxy, CD₃, (1-4C)alkyl, (1-5C)alkoxy, (3-6C)cycloalkyl, (3-6C)cycloalkoxy and (1-6C)alkylcarbonyl; wherein any of said alkyl and alkoxy group is optionally and independently substituted with one, two or three halogen.

In an embodiment, wherein the linker represented by R³ and R⁴ is selected from the group consisting of:

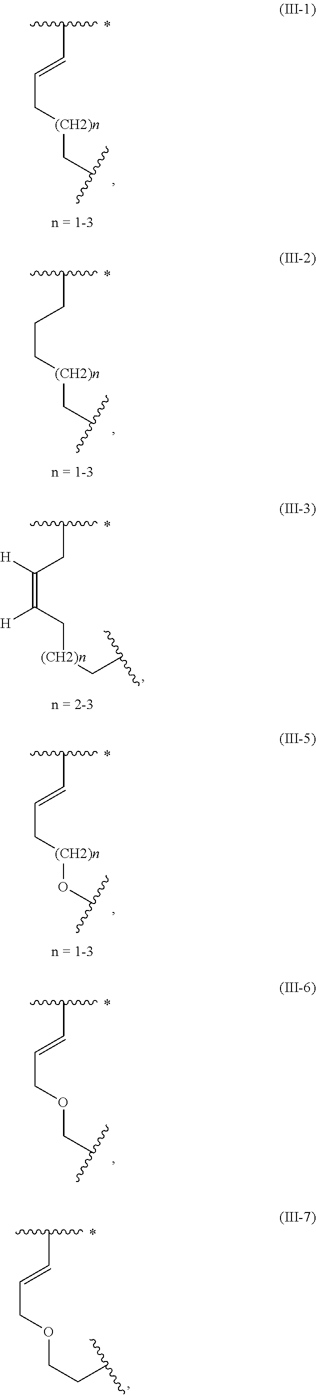

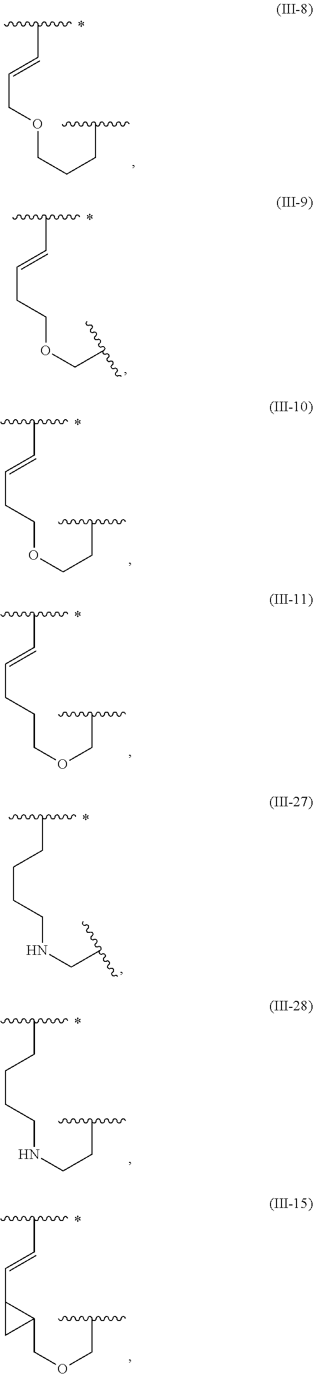

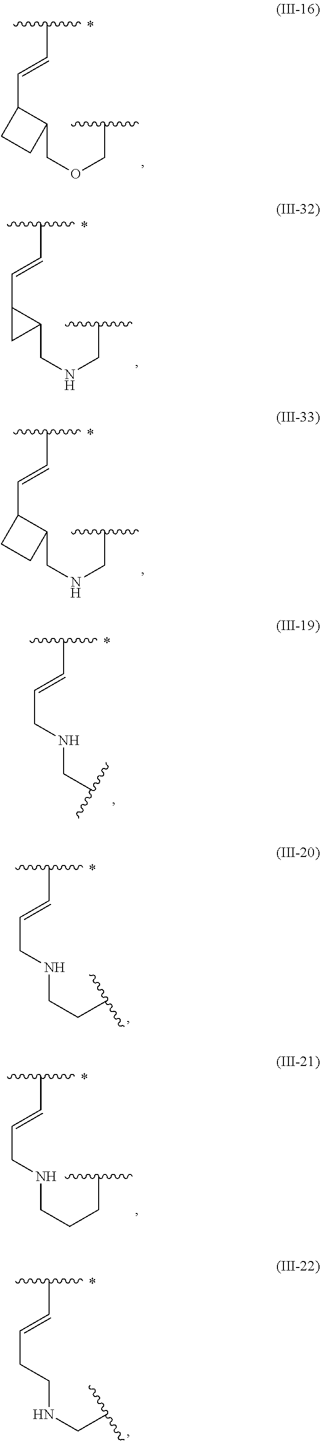

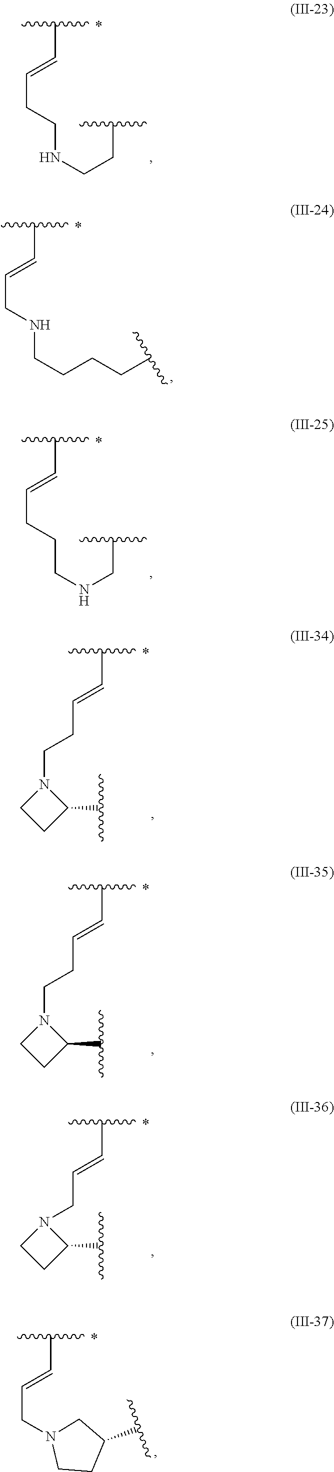

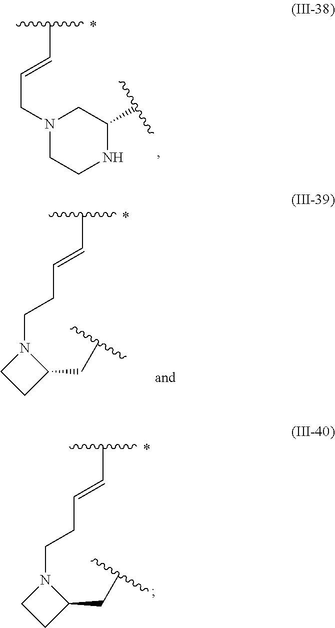

whereby the

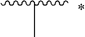

marks the position of R³ in any one of Formula I-a to I-h, and whereby the

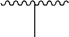

marks the position of R⁴ in any one of Formula II-a to II-f;
wherein any of said linkers is optionally and independently substituted with one or more substituents selected from deuterium, halogen, oxo, hydroxy, CD₃, (1-4C)alkyl, (1-5C)alkoxy, (3-6C)cycloalkyl, (3-6C)cycloalkoxy and (1-6C)alkylcarbonyl; wherein any of said alkyl and alkoxy group is optionally and independently substituted with one, two or three halogen.

In an embodiment, wherein the linker represented by R³ and R⁴ is selected from the group consisting of:

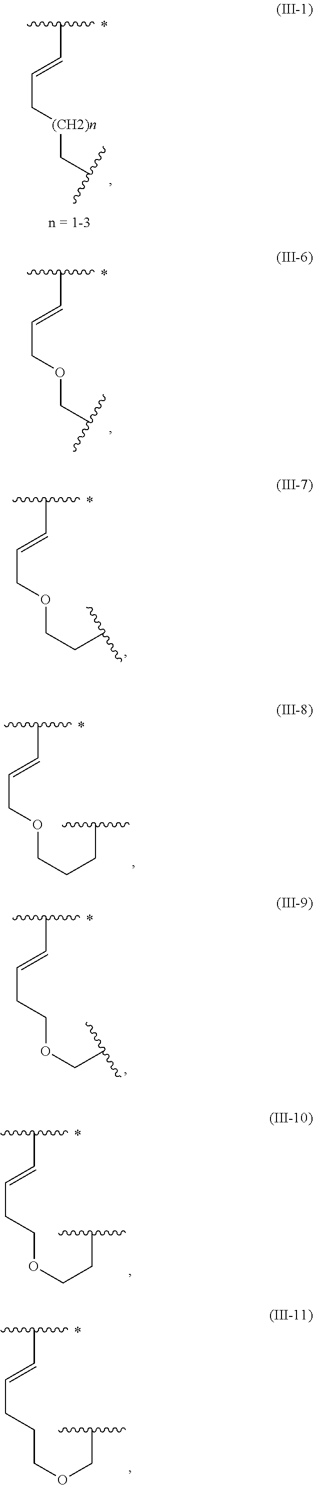

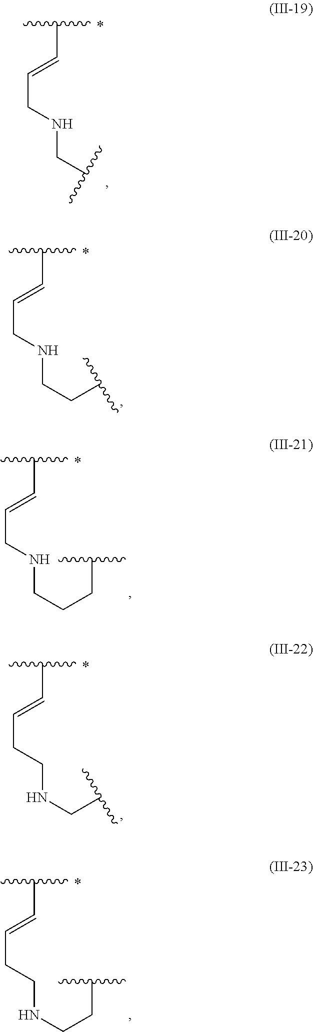 [SMILES: */C=C/CNC* ; */C=C/CNCC* ; */C=C/CNCCC* ; */C=C/CCNC* ; */C=C/CCNCC*]

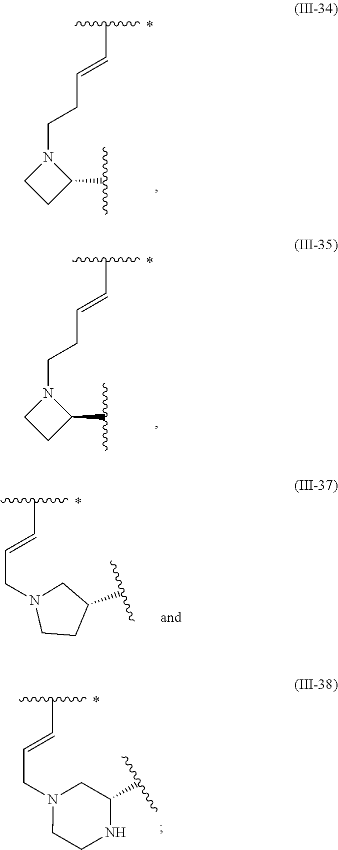 [SMILES: */C=C/CCN1CC[C@H]1* ; */C=C/CCN1CC[C@@H]1* ; */C=C/CN1CC[C@@H](*)C1 ; */C=C/CN1CCN[C@@H](*)C1]

whereby the

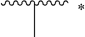

position of R³ in any one of Formula I-a to I-h, and whereby the

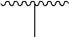

marks the position of R⁴ in any one of Formula II-a to II-f;

wherein any of said linkers is optionally and independently substituted with one or more substituents selected from deuterium, halogen, oxo, hydroxy, CD₃, (1-4C)alkyl, (1-5C)alkoxy, (3-6C)cycloalkyl, (3-6C)cycloalkoxy and (1-6C)alkylcarbonyl; wherein any of said alkyl and alkoxy group is optionally and independently substituted with one, two or three halogen.

In preferred embodiments, a secondary amine group of the linker represented by R³ and R⁴ is substituted by (1-6C)alkylcarbonyl, preferably by methylcarbonyl or ethylcarbonyl. In examples, the secondary amine group of any one of Formula (111-19) to (III-33) or (III-38) may be substituted by (1-4C)alkylcarbonyl, such as by methylcarbonyl or ethylcarbonyl.

In preferred embodiments, a carbon group of the linker represented by R³ and R⁴ is substituted by (1-4)alkyl, preferably by methyl or ethyl, thereby providing a tertiary carbon group.

### (Scaffold)

The compounds of the invention have a scaffold according to any one of Formula (I-a) to (I-h).

BTK inhibitors which do not contain a macrocycle are generally known from the prior art, wherein said known BTK inhibitors have a scaffold according to any one of Formula (I-a) to (I-h):

See for compounds having a scaffold of Formula (I-a): WO 2013/010380, WO2016/210165;

See for compounds having a scaffold of Formula (I-b): Boga S B et al (2017) Bioorg Med Chem Lett, 27, 3939-3943; Liu J et al (2016) ACS Med Chem Lett, 6 198-203;

See for compounds having a scaffold of Formula (I-c): WO 2013/010380;

See for compounds having a scaffold of Formula (I-d): BBA—General Subjects 1864 (2020) 129531;

See for compounds having a scaffold of Formula (I-e): WO 2015/058084, WO2015/095099, WO2015/095102;

See for compounds having a scaffold of Formula (I-f): WO 20130/81016;

See for compounds having a scaffold of Formula (I-g): WO 2017/1 06429, WO 2019/091441, WO 2017/103611;

See for compounds having a scaffold of Formula (I-h): WO 2014/082598, WO 2014/025976.

Furthermore a review showing that various scaffolds have been used in BTK inhibitor compounds is provided in: Yifan Feng, Weiming Duan, Xiaochuan Cu, Chengyuan Liang & Minhang Xin (2019) Bruton's tyrosine kinase (BTK) inhibitors in treating cancer: a patent review (2010-2018), Expert Opinion on Therapeutic Patents, 29:4, 217-241.

All these prior art documents demonstrate that compounds providing BTK inhibition are found for each of the scaffolds according to any one of Formula (I-a) to (I-h).

The present invention concerns novel compounds having a scaffold according to any one of Formula (I-a) to (I-h), and further having a macrocycle as defined according to the embodiments of the invention.

In an embodiment, wherein the compound comprises a bicyclic scaffold according to Formula (I-a) to (I-f) selected from the group consisting of:

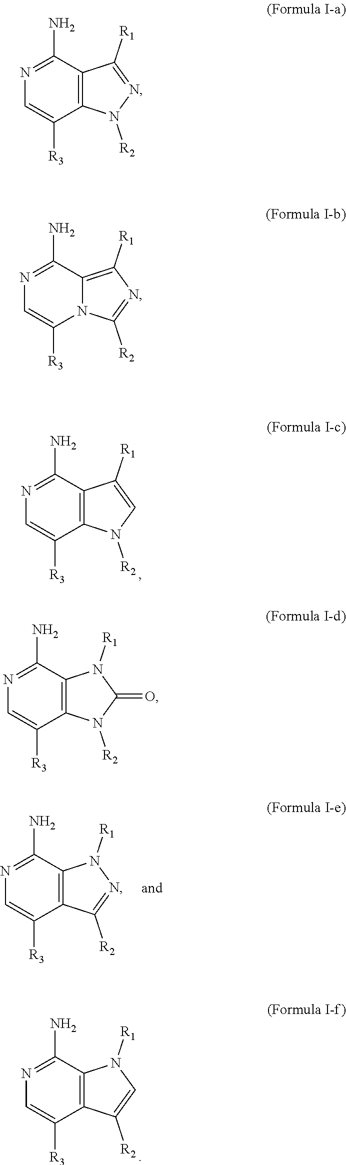

In a preferred embodiment, wherein the compound comprises a bicyclic scaffold selected from:

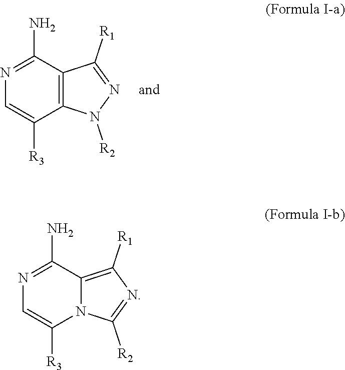

In an embodiment, wherein the compound comprises a monocyclic scaffold according to Formula (I-g) to (I-h) selected from the group consisting of:

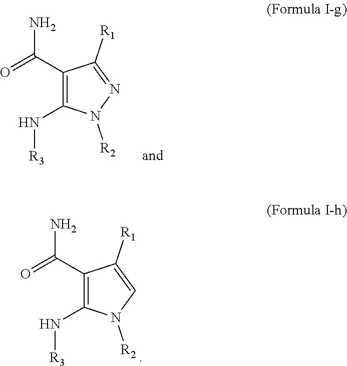

In an alternative embodiment, wherein the compound comprises a scaffold according to any one of Formula (I-a), Formula (I-b), Formula (I-c), Formula (I-d), Formula (I-e), Formula (I-f), Formula (1-g).

In an alternative embodiment, wherein the compound comprises a scaffold according to any one of Formula (I-a), Formula (I-b), Formula (I-c), Formula (I-d), Formula (I-e), Formula (I-f), Formula (I-h).

In an alternative embodiment, wherein the compound comprises a scaffold according to any one of Formula (I-a), Formula (I-b), Formula (I-c), Formula (I-d), Formula (I-e), Formula (1-g), Formula (1-h).

In an alternative embodiment, wherein the compound comprises a scaffold according to any one of Formula (I-a), Formula (I-b), Formula (I-c), Formula (I-d), Formula (I-f), Formula (I-g), Formula (I-h).

In an alternative embodiment, wherein the compound comprises a scaffold according to any one of Formula (I-a), Formula (I-b), Formula (I-c), Formula (I-e), Formula (I-f), Formula (I-g), Formula (I-h).

In an alternative embodiment, wherein the compound comprises a scaffold according to any one of Formula (I-a), Formula (I-b), Formula (I-d), Formula (I-e), Formula (I-f), Formula (I-g), Formula (I-h).

In an alternative embodiment, wherein the compound comprises a scaffold according to any one of Formula (I-a), Formula (I-c), Formula (I-d), Formula (I-e), Formula (I-f), Formula (I-g), Formula (I-h).

In an alternative embodiment, wherein the compound comprises a scaffold according to any one of Formula (I-b), Formula (I-c), Formula (I-d), Formula (I-e), Formula (I-f), Formula (I-g), Formula (I-h).

### (Specific Compounds)

In an embodiment, wherein the compound has a sub-formula (1-226) selected from the group consisting of:

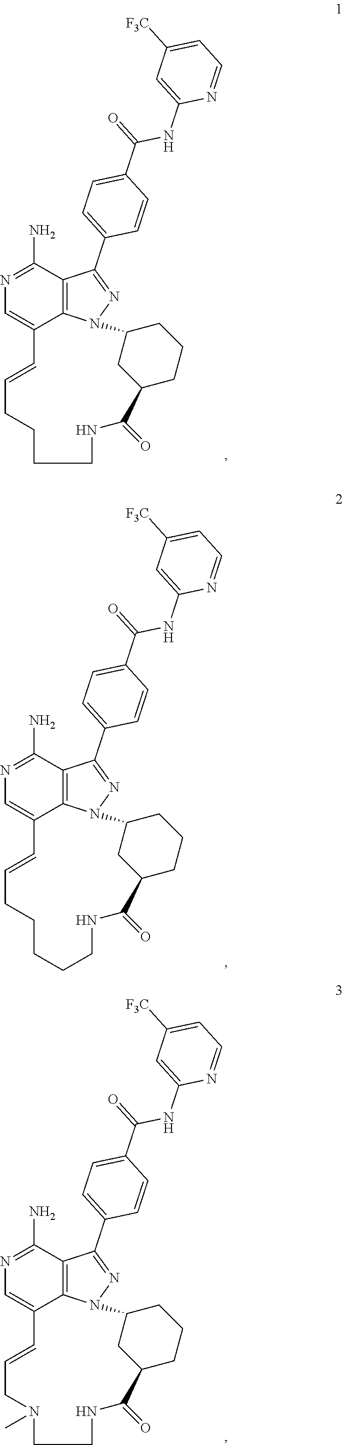 [SMILES: Nc1ncc2c3c1c(-c1ccc(C(=O)Nc4cc(C(F)(F)F)ccn4)cc1)nn3[C@@H]1CCC[C@H](C1)C(=O)NCCCC/C=C/2 ; Nc1ncc2c3c1c(-c1ccc(C(=O)Nc4cc(C(F)(F)F)ccn4)cc1)nn3[C@@H]1CCC[C@H](C1)C(=O)NCCCCC/C=C/2 ; CN1C/C=C/c2cnc(N)c3c(-c4ccc(C(=O)Nc5cc(C(F)(F)F)ccn5)cc4)nn(c23)[C@@H]2CCC[C@H](C2)C(=O)NCC1]

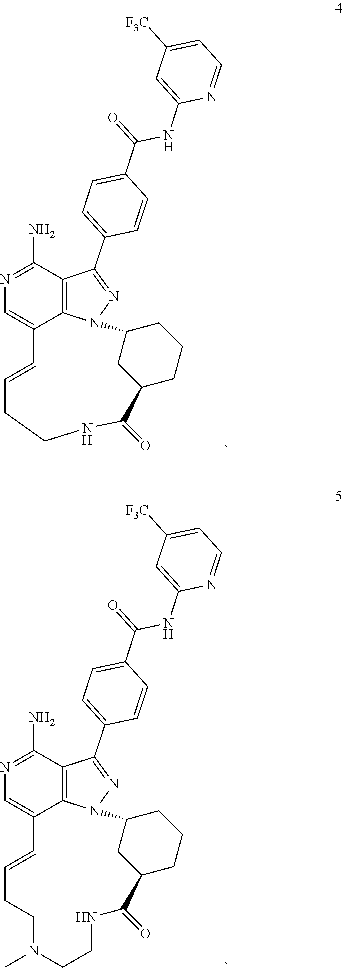

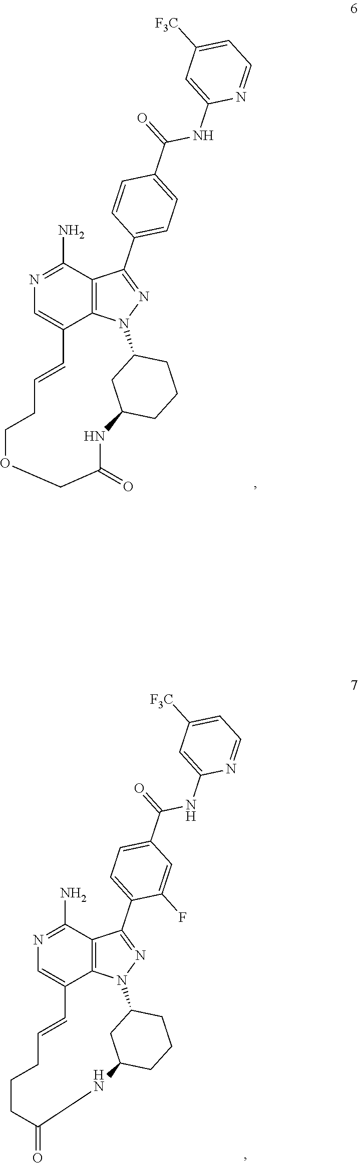 [SMILES: Nc1ncc2c3c1c(-c1ccc(C(=O)Nc4cc(C(F)(F)F)ccn4)cc1)nn3[C@@H]1CCC[C@H](C1)NC(=O)COCC/C=C/2 ; Nc1ncc2c3c1c(-c1ccc(C(=O)Nc4cc(C(F)(F)F)ccn4)cc1F)nn3[C@@H]1CCC[C@H](C1)NC(=O)CCC/C=C/2]

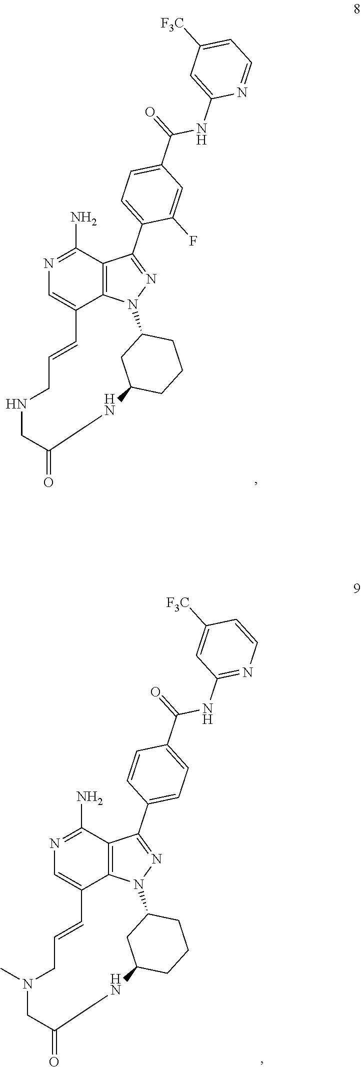 [SMILES: Nc1ncc2c3c1c(-c1ccc(C(=O)Nc4cc(C(F)(F)F)ccn4)cc1F)nn3[C@@H]1CCC[C@H](C1)NC(=O)CNC/C=C/2 ; CN1C/C=C/c2cnc(N)c3c(-c4ccc(C(=O)Nc5cc(C(F)(F)F)ccn5)cc4)nn(c23)[C@@H]2CCC[C@H](C2)NC(=O)C1]

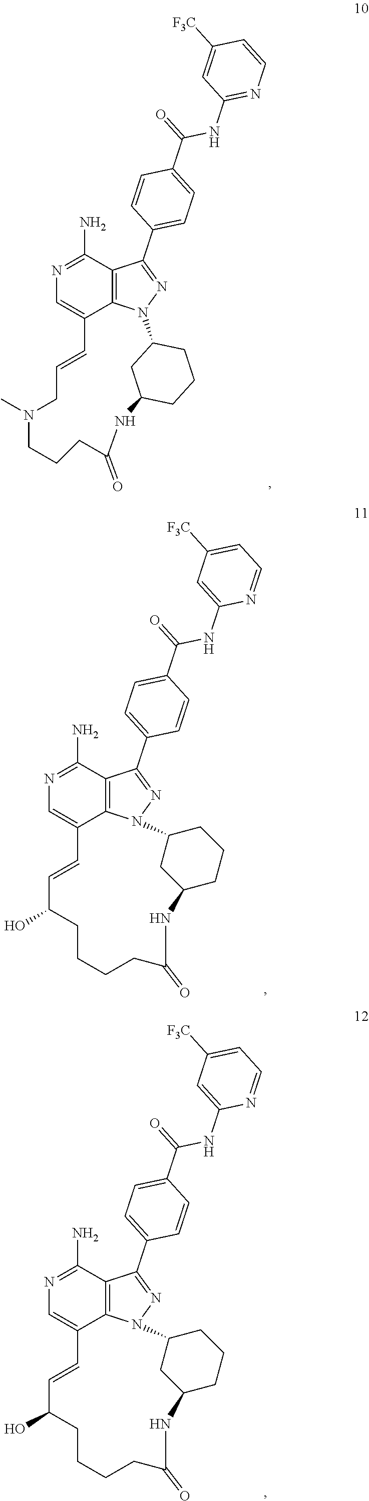

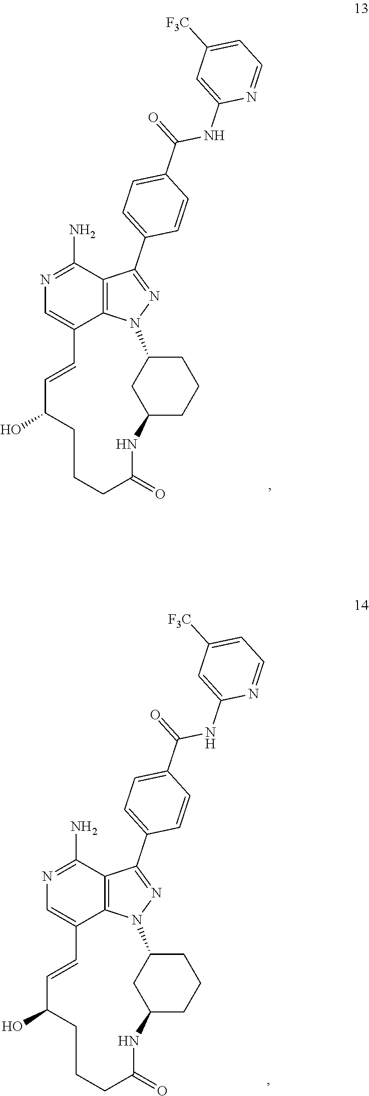 [SMILES: Nc1ncc2c3c1c(-c1ccc(C(=O)Nc4cc(C(F)(F)F)ccn4)cc1)nn3[C@@H]1CCC[C@H](C1)NC(=O)CCC[C@H](O)/C=C/2 ; Nc1ncc2c3c1c(-c1ccc(C(=O)Nc4cc(C(F)(F)F)ccn4)cc1)nn3[C@@H]1CCC[C@H](C1)NC(=O)CCC[C@@H](O)/C=C/2]

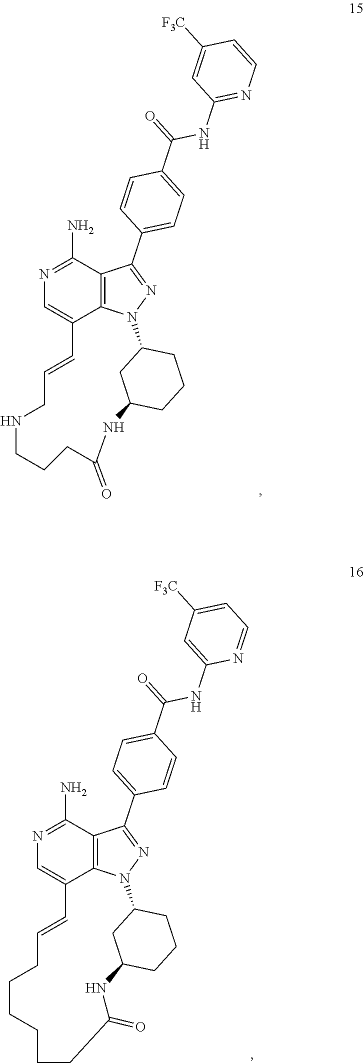 [SMILES: Nc1ncc2c3c1c(-c1ccc(C(=O)Nc4cc(C(F)(F)F)ccn4)cc1)nn3[C@@H]1CCC[C@H](C1)NC(=O)CCCNC/C=C/2 ; Nc1ncc2c3c1c(-c1ccc(C(=O)Nc4cc(C(F)(F)F)ccn4)cc1)nn3[C@@H]1CCC[C@H](C1)NC(=O)CCCCCC/C=C/2]

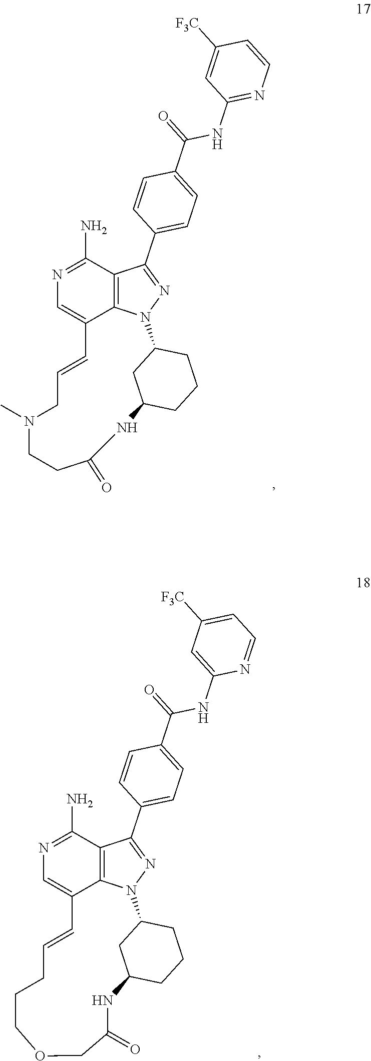 [SMILES: CN1C/C=C/c2cnc(N)c3c(-c4ccc(C(=O)Nc5cc(C(F)(F)F)ccn5)cc4)nn(c23)[C@@H]2CCC[C@H](C2)NC(=O)CC1 ; Nc1ncc2c3c1c(-c1ccc(C(=O)Nc4cc(C(F)(F)F)ccn4)cc1)nn3[C@@H]1CCC[C@H](C1)NC(=O)COCCC/C=C/2]

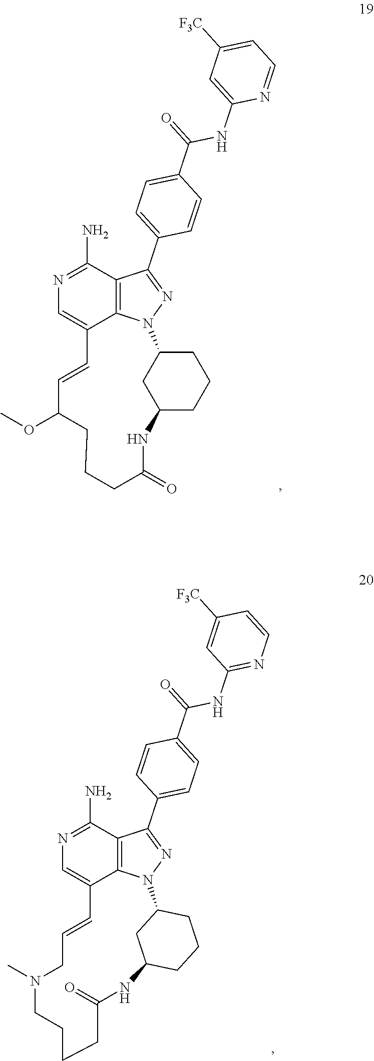 [SMILES: COC1/C=C/c2cnc(N)c3c(-c4ccc(C(=O)Nc5cc(C(F)(F)F)ccn5)cc4)nn(c23)[C@@H]2CCC[C@H](C2)NC(=O)CCC1 ; CN1C/C=C/c2cnc(N)c3c(-c4ccc(C(=O)Nc5cc(C(F)(F)F)ccn5)cc4)nn(c23)[C@@H]2CCC[C@H](C2)NC(=O)CCCC1]

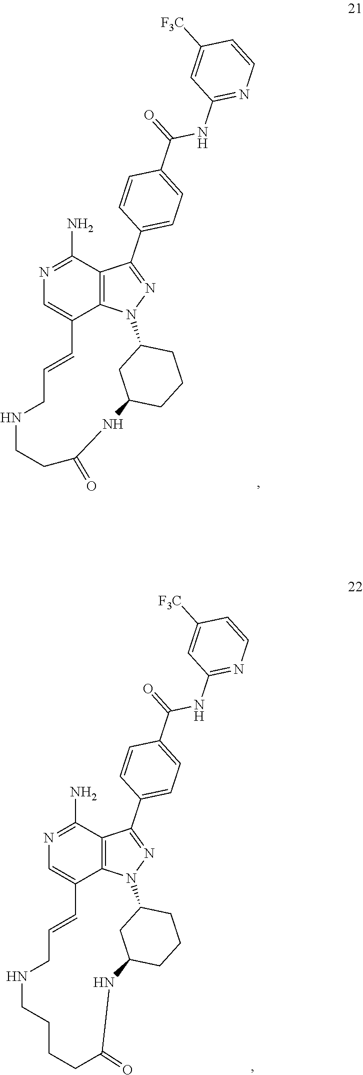 [SMILES: Nc1ncc2c3c1c(-c1ccc(C(=O)Nc4cc(C(F)(F)F)ccn4)cc1)nn3[C@@H]1CCC[C@H](C1)NC(=O)CCNC/C=C/2 ; Nc1ncc2c3c1c(-c1ccc(C(=O)Nc4cc(C(F)(F)F)ccn4)cc1)nn3[C@@H]1CCC[C@H](C1)NC(=O)CCCCNC/C=C/2]

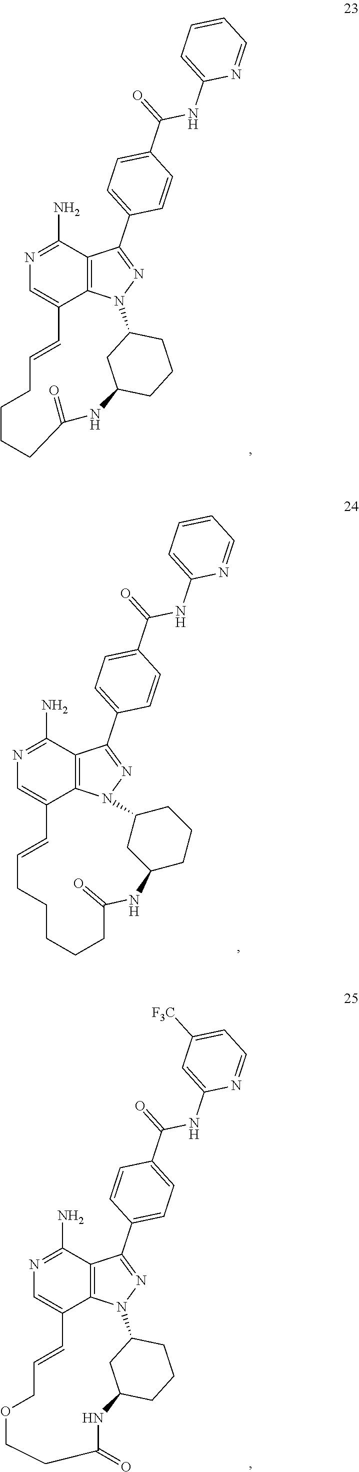 [SMILES: Nc1ncc2c3c1c(-c1ccc(C(=O)Nc4ccccn4)cc1)nn3[C@@H]1CCC[C@H](C1)NC(=O)CCCC/C=C/2 ; Nc1ncc2c3c1c(-c1ccc(C(=O)Nc4ccccn4)cc1)nn3[C@@H]1CCC[C@H](C1)NC(=O)CCCCC/C=C/2 ; Nc1ncc2c3c1c(-c1ccc(C(=O)Nc4cc(C(F)(F)F)ccn4)cc1)nn3[C@@H]1CCC[C@H](C1)NC(=O)CCOC/C=C/2]

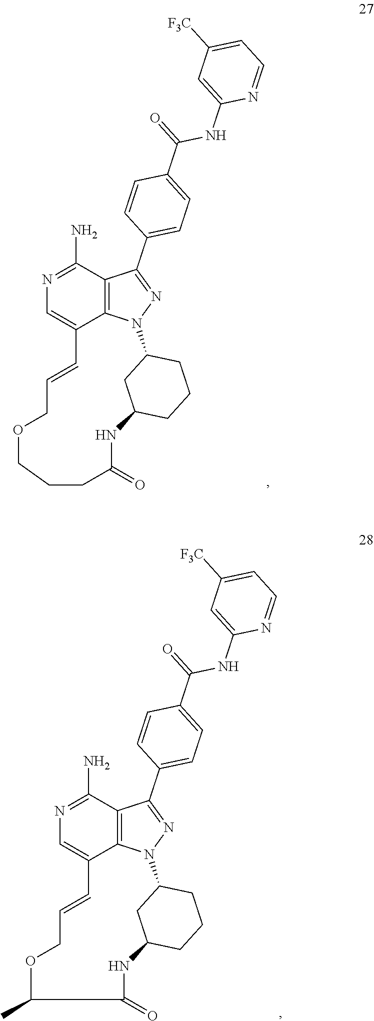 [SMILES: Nc1ncc2c3c1c(-c1ccc(C(=O)Nc4cc(C(F)(F)F)ccn4)cc1)nn3[C@@H]1CCC[C@H](C1)NC(=O)CCCOC/C=C/2 ; C[C@H]1OC/C=C/c2cnc(N)c3c(-c4ccc(C(=O)Nc5cc(C(F)(F)F)ccn5)cc4)nn(c23)[C@@H]2CCC[C@H](C2)NC1=O]

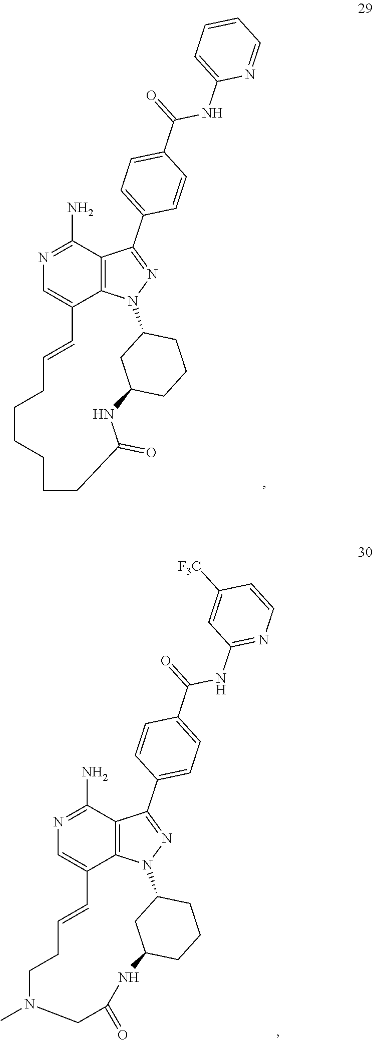 [SMILES: Nc1ncc2c3c1c(-c1ccc(C(=O)Nc4ccccn4)cc1)nn3[C@@H]1CCC[C@H](C1)NC(=O)CCCCCC/C=C/2 ; CN1CC/C=C/c2cnc(N)c3c(-c4ccc(C(=O)Nc5cc(C(F)(F)F)ccn5)cc4)nn(c23)[C@@H]2CCC[C@H](C2)NC(=O)C1]

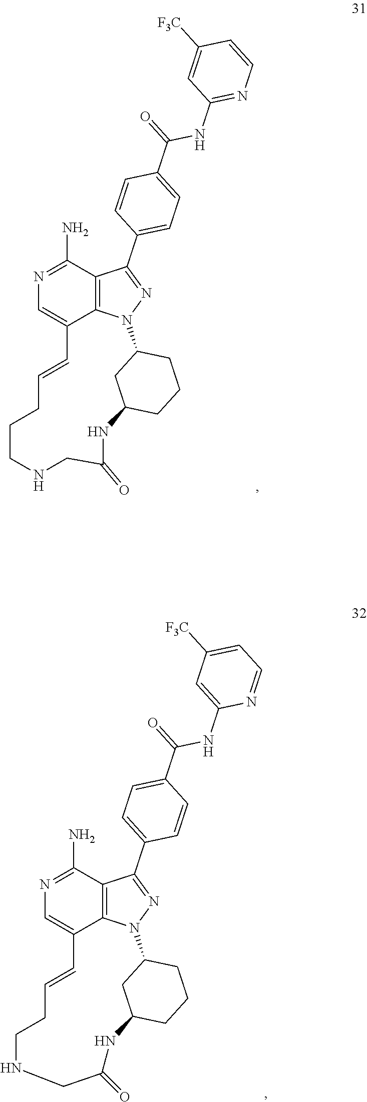 [SMILES: Nc1ncc2c3c1c(-c1ccc(C(=O)Nc4cc(C(F)(F)F)ccn4)cc1)nn3[C@@H]1CCC[C@H](C1)NC(=O)CNCCC/C=C/2 ; Nc1ncc2c3c1c(-c1ccc(C(=O)Nc4cc(C(F)(F)F)ccn4)cc1)nn3[C@@H]1CCC[C@H](C1)NC(=O)CNCC/C=C/2]

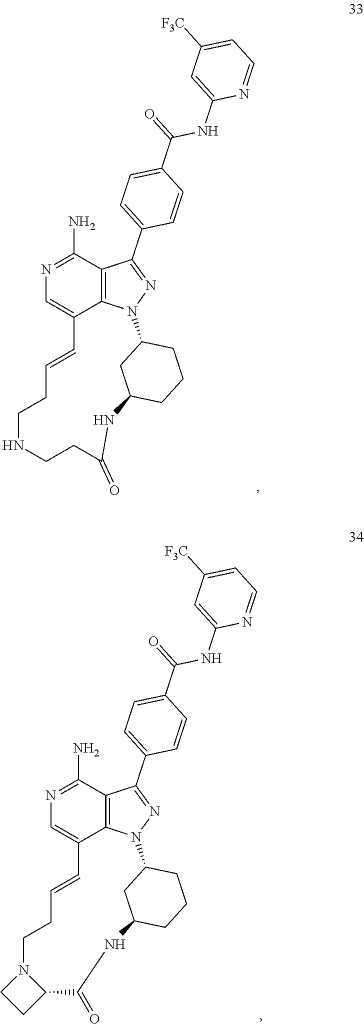 [SMILES: Nc1ncc2c3c1c(-c1ccc(C(=O)Nc4cc(C(F)(F)F)ccn4)cc1)nn3[C@@H]1CCC[C@H](C1)NC(=O)CCNCC/C=C/2 ; Nc1ncc2c3c1c(-c1ccc(C(=O)Nc4cc(C(F)(F)F)ccn4)cc1)nn3[C@@H]1CCC[C@H](C1)NC(=O)[C@@H]1CCN1CC/C=C/2]

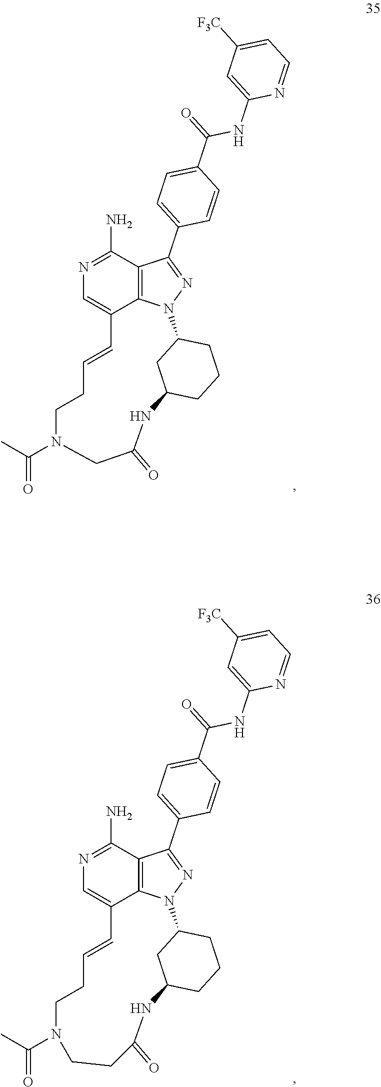 [SMILES: CC(=O)N1CC/C=C/c2cnc(N)c3c(-c4ccc(C(=O)Nc5cc(C(F)(F)F)ccn5)cc4)nn(c23)[C@@H]2CCC[C@H](C2)NC(=O)C1 ; CC(=O)N1CC/C=C/c2cnc(N)c3c(-c4ccc(C(=O)Nc5cc(C(F)(F)F)ccn5)cc4)nn(c23)[C@@H]2CCC[C@H](C2)NC(=O)CC1]

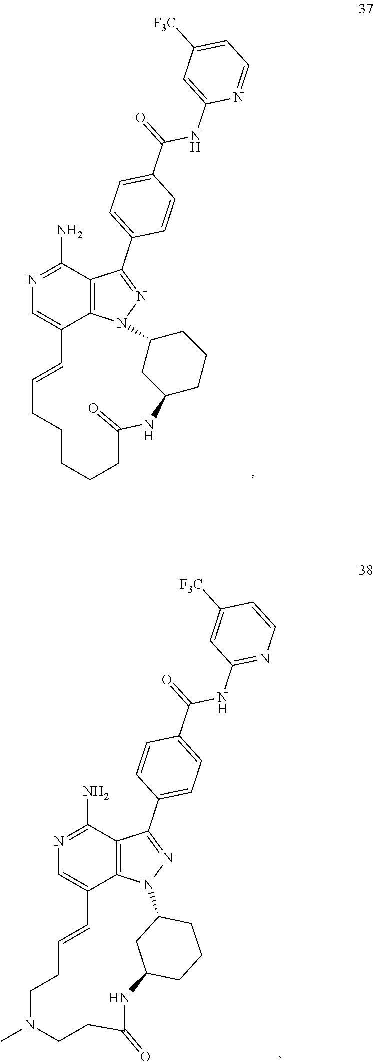 [SMILES: Nc1ncc2c3c1c(-c1ccc(C(=O)Nc4cc(C(F)(F)F)ccn4)cc1)nn3[C@@H]1CCC[C@H](C1)NC(=O)CCCCC/C=C/2 ; CN1CC/C=C/c2cnc(N)c3c(-c4ccc(C(=O)Nc5cc(C(F)(F)F)ccn5)cc4)nn(c23)[C@@H]2CCC[C@H](C2)NC(=O)CC1]

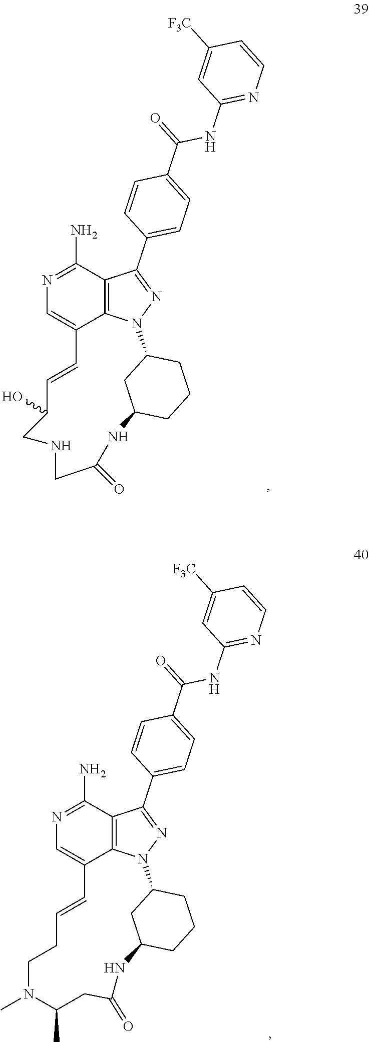 [SMILES: Nc1ncc2c3c1c(-c1ccc(C(=O)Nc4cc(C(F)(F)F)ccn4)cc1)nn3[C@@H]1CCC[C@H](C1)NC(=O)CNCC(O)/C=C/2 ; C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCN1C]

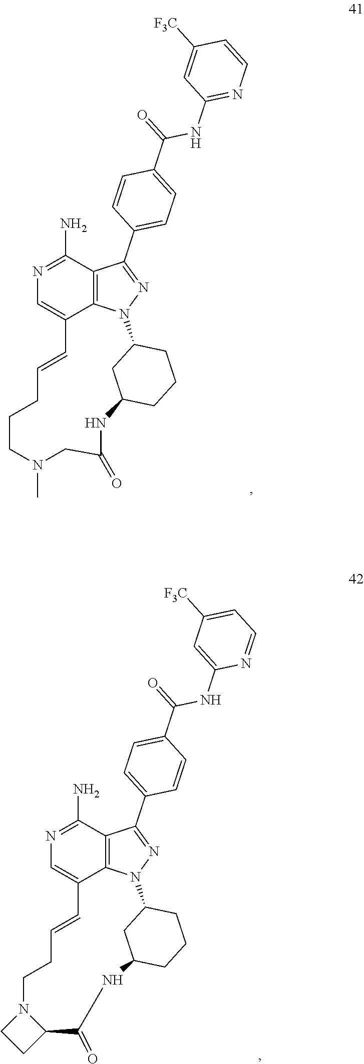 [SMILES: CN1CCC/C=C/c2cnc(N)c3c(-c4ccc(C(=O)Nc5cc(C(F)(F)F)ccn5)cc4)nn(c23)[C@@H]2CCC[C@H](C2)NC(=O)C1 ; Nc1ncc2c3c1c(-c1ccc(C(=O)Nc4cc(C(F)(F)F)ccn4)cc1)nn3[C@@H]1CCC[C@H](C1)NC(=O)[C@H]1CCN1CC/C=C/2]

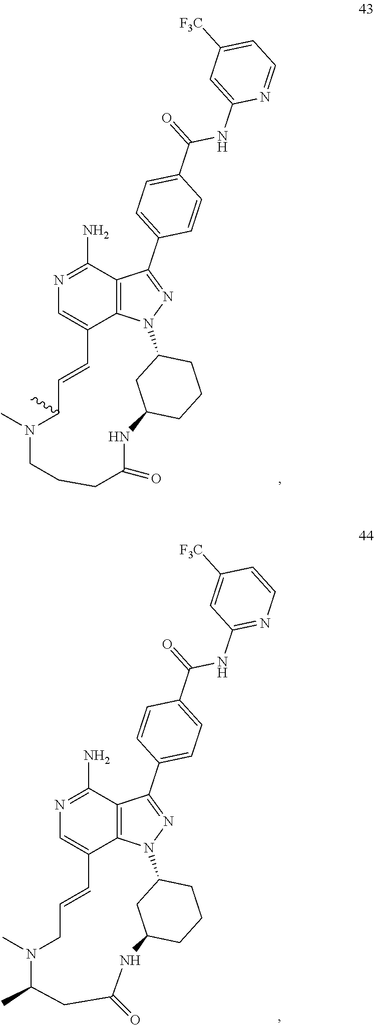 [SMILES: CC1/C=C/c2cnc(N)c3c(-c4ccc(C(=O)Nc5cc(C(F)(F)F)ccn5)cc4)nn(c23)[C@@H]2CCC[C@H](C2)NC(=O)CCCN1C ; C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CN1C]

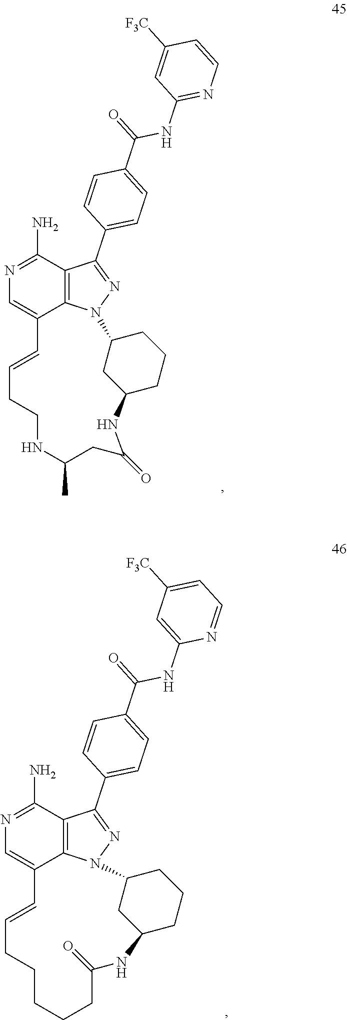 [SMILES: C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCN1 ; Nc1ncc2c3c1c(-c1ccc(C(=O)Nc4cc(C(F)(F)F)ccn4)cc1)nn3[C@@H]1CCC[C@H](C1)NC(=O)CCCCC/C=C/2]

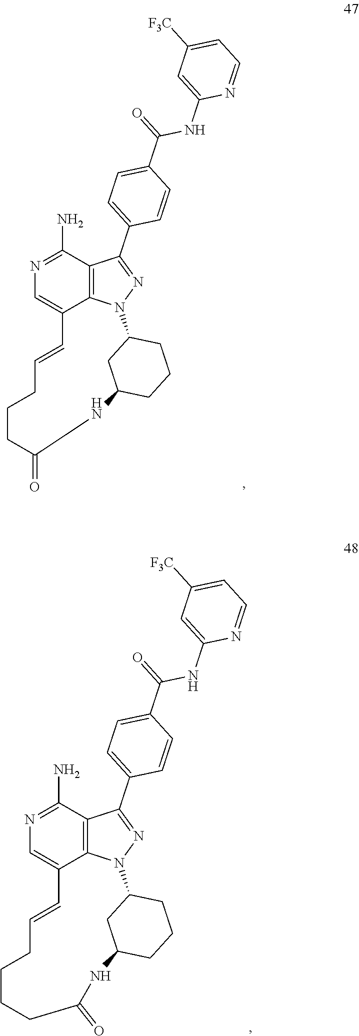 [SMILES: Nc1ncc2c3c1c(-c1ccc(C(=O)Nc4cc(C(F)(F)F)ccn4)cc1)nn3[C@@H]1CCC[C@H](C1)NC(=O)CCC/C=C/2 ; Nc1ncc2c3c1c(-c1ccc(C(=O)Nc4cc(C(F)(F)F)ccn4)cc1)nn3[C@@H]1CCC[C@H](C1)NC(=O)CCCC/C=C/2]

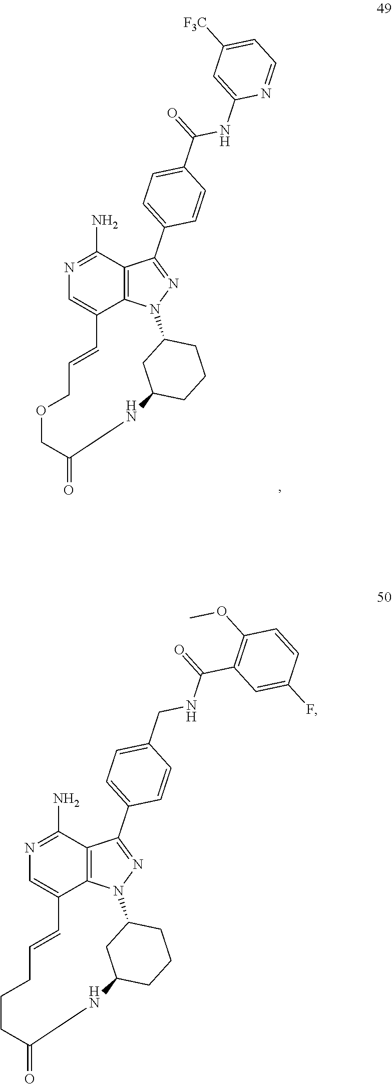 [SMILES: Nc1ncc2c3c1c(-c1ccc(C(=O)Nc4cc(C(F)(F)F)ccn4)cc1)nn3[C@@H]1CCC[C@H](C1)NC(=O)COC/C=C/2 ; COc1ccc(F)cc1C(=O)NCc1ccc(-c2nn3c4c(cnc(N)c24)/C=C/CCCC(=O)N[C@@H]2CCC[C@@H]3C2)cc1]

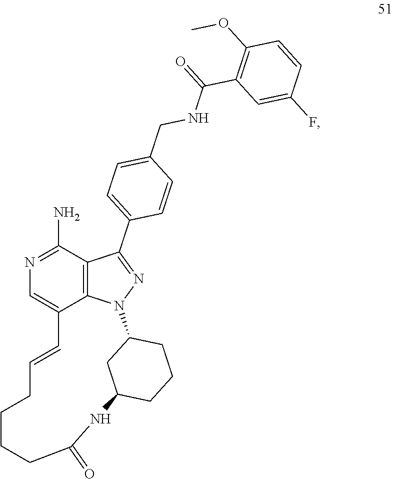 [SMILES: COc1ccc(F)cc1C(=O)NCc1ccc(-c2nn3c4c(cnc(N)c24)/C=C/CCCCC(=O)N[C@@H]2CCC[C@@H]3C2)cc1]

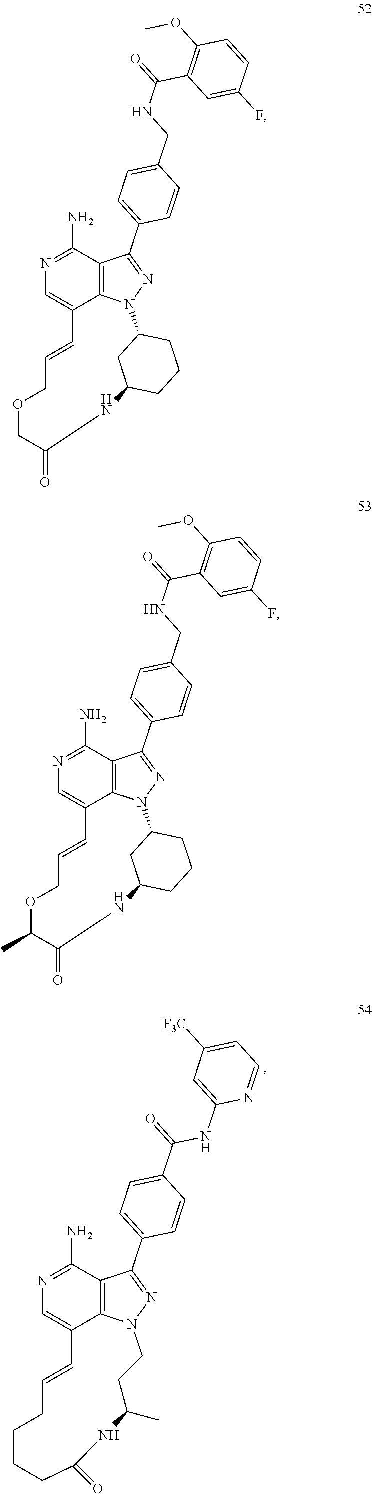 [SMILES: COc1ccc(F)cc1C(=O)NCc1ccc(-c2nn3c4c(cnc(N)c24)/C=C/COCC(=O)N[C@@H]2CCC[C@@H]3C2)cc1 ; COc1ccc(F)cc1C(=O)NCc1ccc(-c2nn3c4c(cnc(N)c24)/C=C/CO[C@H](C)C(=O)N[C@@H]2CCC[C@@H]3C2)cc1 ; C[C@@H]1CCn2nc(-c3ccc(C(=O)Nc4cc(C(F)(F)F)ccn4)cc3)c3c(N)ncc(c32)/C=C/CCCCC(=O)N1]

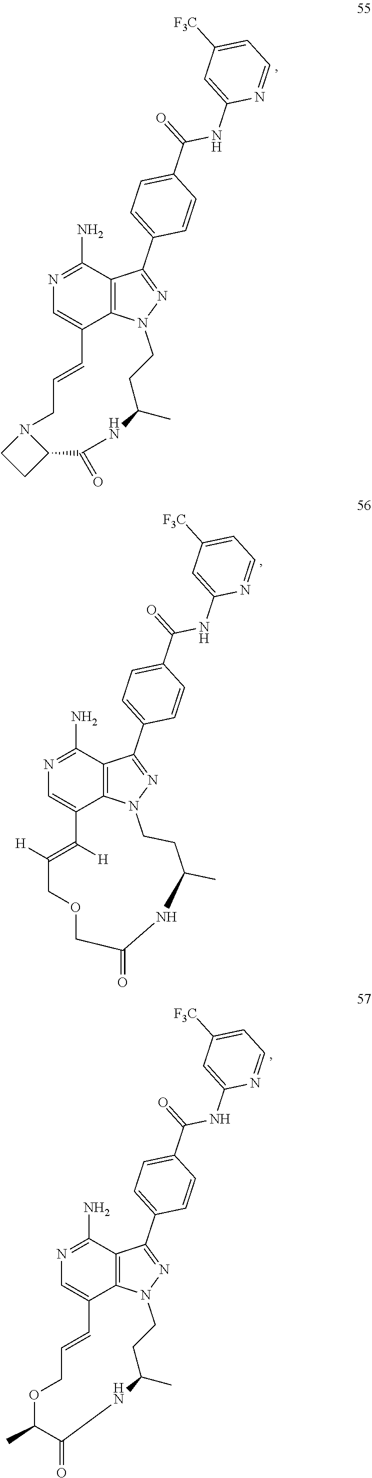 [SMILES: C[C@@H]1CCn2nc(-c3ccc(C(=O)Nc4cc(C(F)(F)F)ccn4)cc3)c3c(N)ncc(c32)/C=C/CN2CC[C@H]2C(=O)N1 ; C[C@@H]1CCn2nc(-c3ccc(C(=O)Nc4cc(C(F)(F)F)ccn4)cc3)c3c(N)ncc(c32)/C=C/COCC(=O)N1 ; C[C@@H]1CCn2nc(-c3ccc(C(=O)Nc4cc(C(F)(F)F)ccn4)cc3)c3c(N)ncc(c32)/C=C/CO[C@H](C)C(=O)N1]

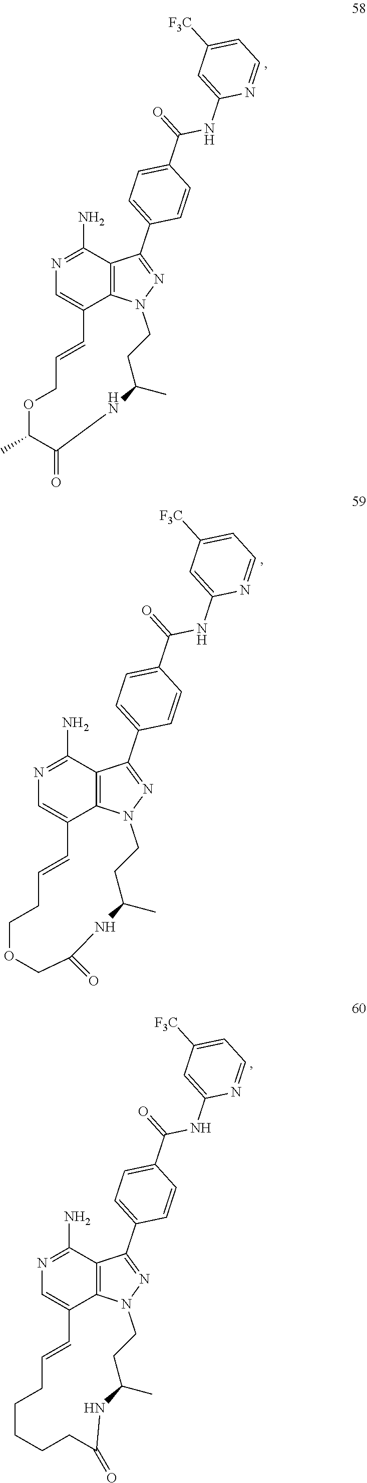 [SMILES: C[C@@H]1CCn2nc(-c3ccc(C(=O)Nc4cc(C(F)(F)F)ccn4)cc3)c3c(N)ncc(c32)/C=C/CO[C@@H](C)C(=O)N1 ; C[C@@H]1CCn2nc(-c3ccc(C(=O)Nc4cc(C(F)(F)F)ccn4)cc3)c3c(N)ncc(c32)/C=C/CCOCC(=O)N1 ; C[C@@H]1CCn2nc(-c3ccc(C(=O)Nc4cc(C(F)(F)F)ccn4)cc3)c3c(N)ncc(c32)/C=C/CCCCCC(=O)N1]

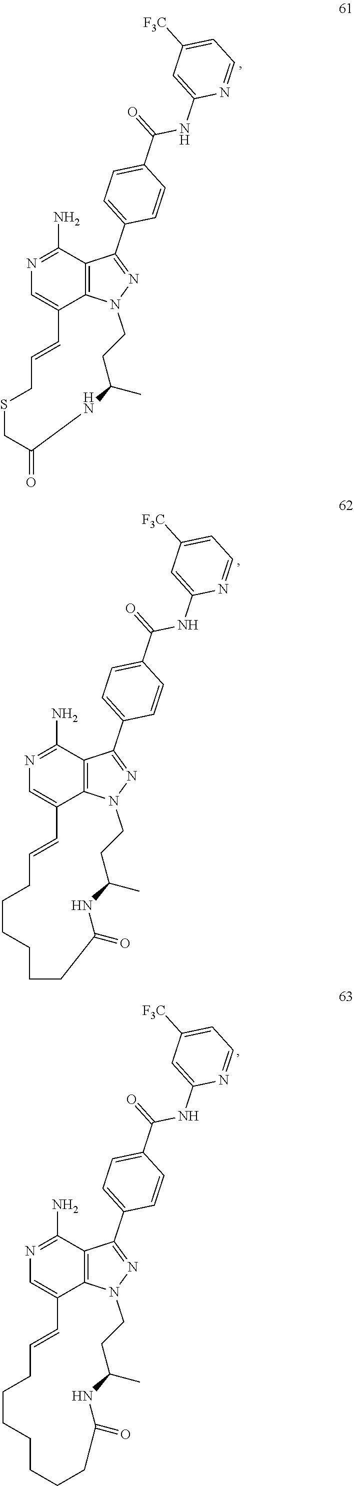 [SMILES: C[C@@H]1CCn2nc(-c3ccc(C(=O)Nc4cc(C(F)(F)F)ccn4)cc3)c3c(N)ncc(c32)/C=C/CSCC(=O)N1 ; C[C@@H]1CCn2nc(-c3ccc(C(=O)Nc4cc(C(F)(F)F)ccn4)cc3)c3c(N)ncc(c32)/C=C/CCCCCCC(=O)N1 ; C[C@@H]1CCn2nc(-c3ccc(C(=O)Nc4cc(C(F)(F)F)ccn4)cc3)c3c(N)ncc(c32)/C=C/CCCCCCCC(=O)N1]

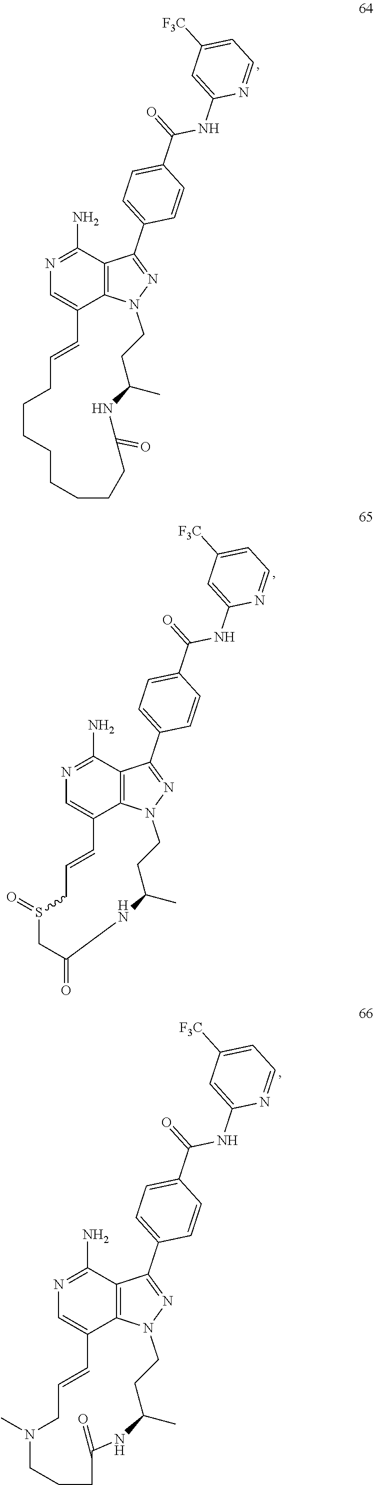 [SMILES: C[C@@H]1CCn2nc(-c3ccc(C(=O)Nc4cc(C(F)(F)F)ccn4)cc3)c3c(N)ncc(c32)/C=C/CCCCCCCCC(=O)N1 ; C[C@@H]1CCn2nc(-c3ccc(C(=O)Nc4cc(C(F)(F)F)ccn4)cc3)c3c(N)ncc(c32)/C=C/CS(=O)CC(=O)N1 ; C[C@@H]1CCn2nc(-c3ccc(C(=O)Nc4cc(C(F)(F)F)ccn4)cc3)c3c(N)ncc(c32)/C=C/CN(C)CCCC(=O)N1]

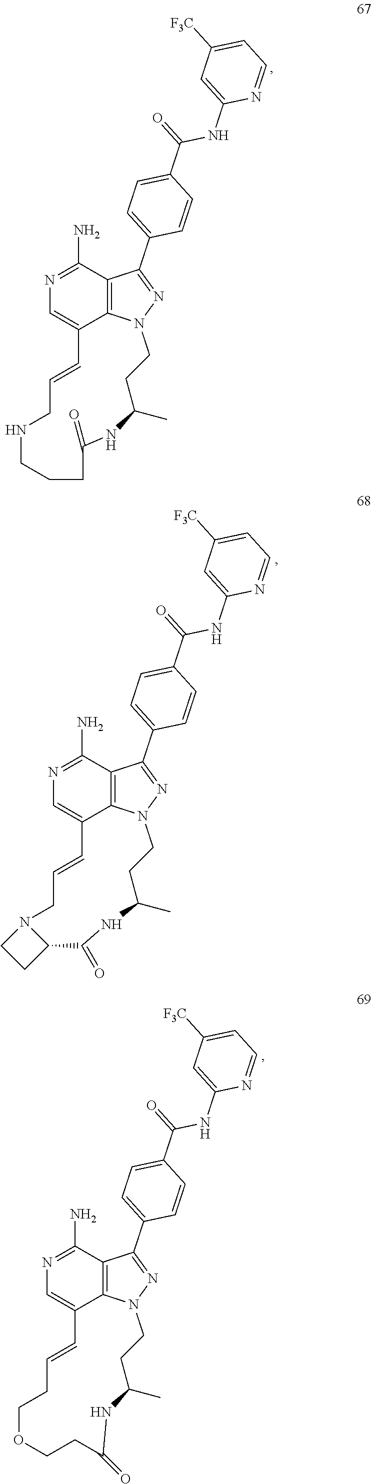 [SMILES: C[C@@H]1CCn2nc(-c3ccc(C(=O)Nc4cc(C(F)(F)F)ccn4)cc3)c3c(N)ncc(c32)/C=C/CNCCCC(=O)N1 ; C[C@@H]1CCn2nc(-c3ccc(C(=O)Nc4cc(C(F)(F)F)ccn4)cc3)c3c(N)ncc(c32)/C=C/CN2CC[C@H]2C(=O)N1 ; C[C@@H]1CCn2nc(-c3ccc(C(=O)Nc4cc(C(F)(F)F)ccn4)cc3)c3c(N)ncc(c32)/C=C/CCOCCC(=O)N1]

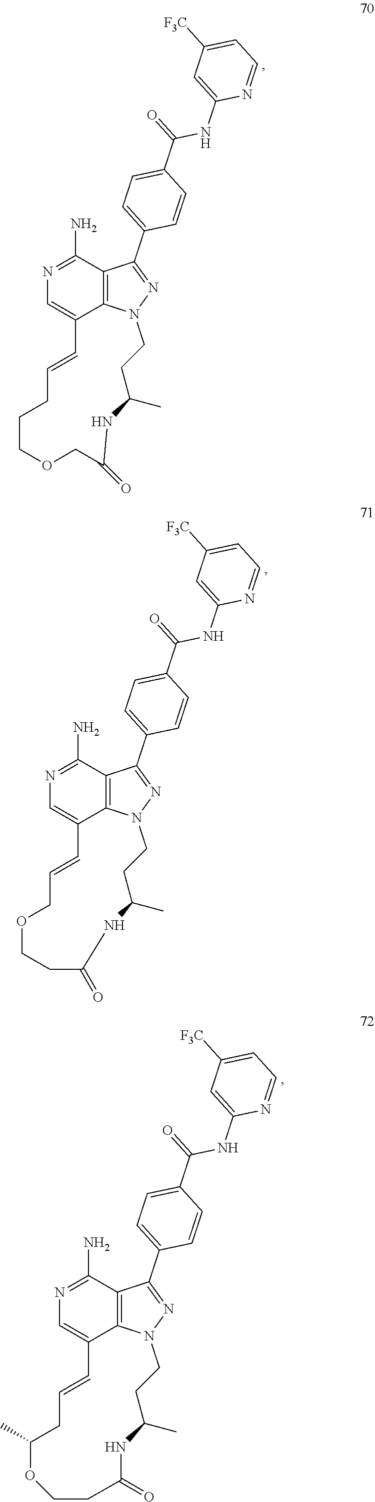 [SMILES: C[C@@H]1CCn2nc(-c3ccc(C(=O)Nc4cc(C(F)(F)F)ccn4)cc3)c3c(N)ncc(c32)/C=C/CCCOCC(=O)N1 ; C[C@@H]1CCn2nc(-c3ccc(C(=O)Nc4cc(C(F)(F)F)ccn4)cc3)c3c(N)ncc(c32)/C=C/COCCC(=O)N1 ; C[C@@H]1CCn2nc(-c3ccc(C(=O)Nc4cc(C(F)(F)F)ccn4)cc3)c3c(N)ncc(c32)/C=C/C[C@@H](C)OCCC(=O)N1]

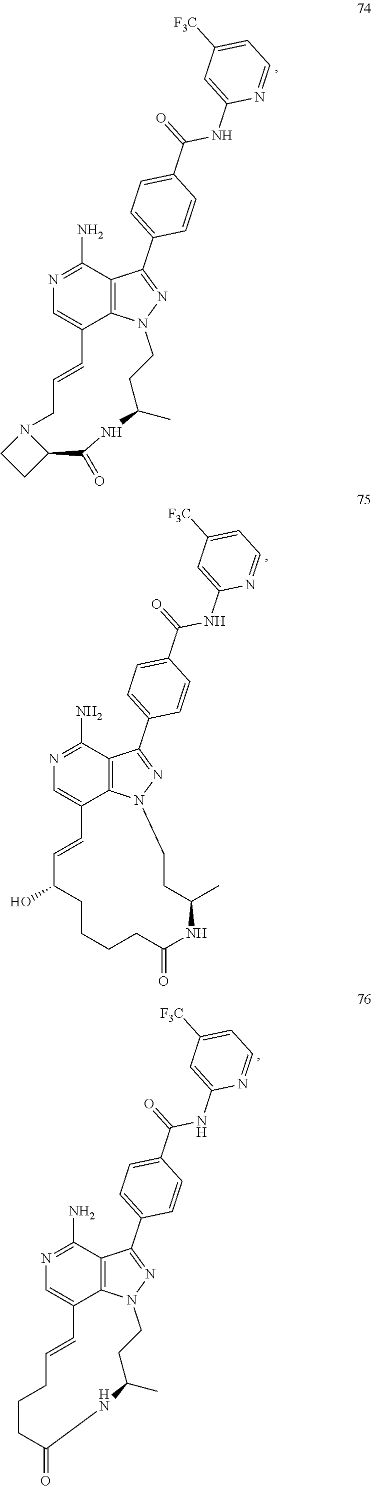

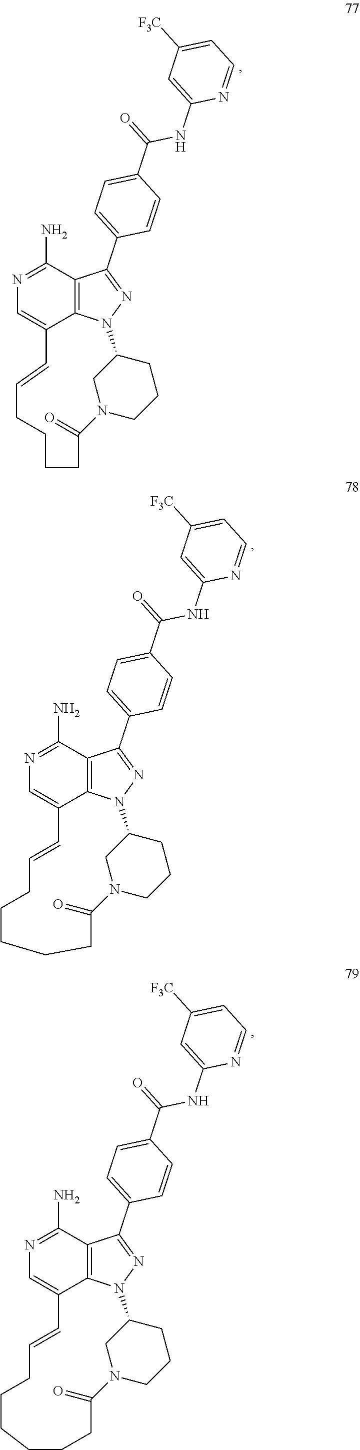 [SMILES: Nc1ncc2c3c1c(-c1ccc(C(=O)Nc4cc(C(F)(F)F)ccn4)cc1)nn3[C@@H]1CCCN(C1)C(=O)CCCC/C=C/2 ; Nc1ncc2c3c1c(-c1ccc(C(=O)Nc4cc(C(F)(F)F)ccn4)cc1)nn3[C@@H]1CCCN(C1)C(=O)CCCCC/C=C/2 ; Nc1ncc2c3c1c(-c1ccc(C(=O)Nc4cc(C(F)(F)F)ccn4)cc1)nn3[C@@H]1CCCN(C1)C(=O)CCCCCC/C=C/2]

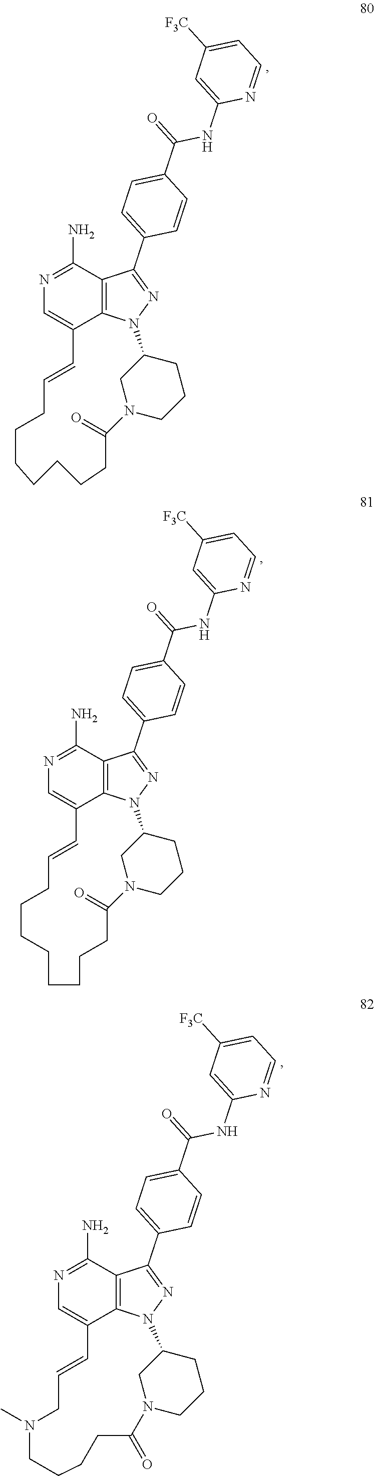 [SMILES: Nc1ncc2c3c1c(-c1ccc(C(=O)Nc4cc(C(F)(F)F)ccn4)cc1)nn3[C@@H]1CCCN(C1)C(=O)CCCCCCC/C=C/2 ; Nc1ncc2c3c1c(-c1ccc(C(=O)Nc4cc(C(F)(F)F)ccn4)cc1)nn3[C@@H]1CCCN(C1)C(=O)CCCCCCCC/C=C/2 ; CN1C/C=C/c2cnc(N)c3c(-c4ccc(C(=O)Nc5cc(C(F)(F)F)ccn5)cc4)nn(c23)[C@@H]2CCCN(C2)C(=O)CCCC1]

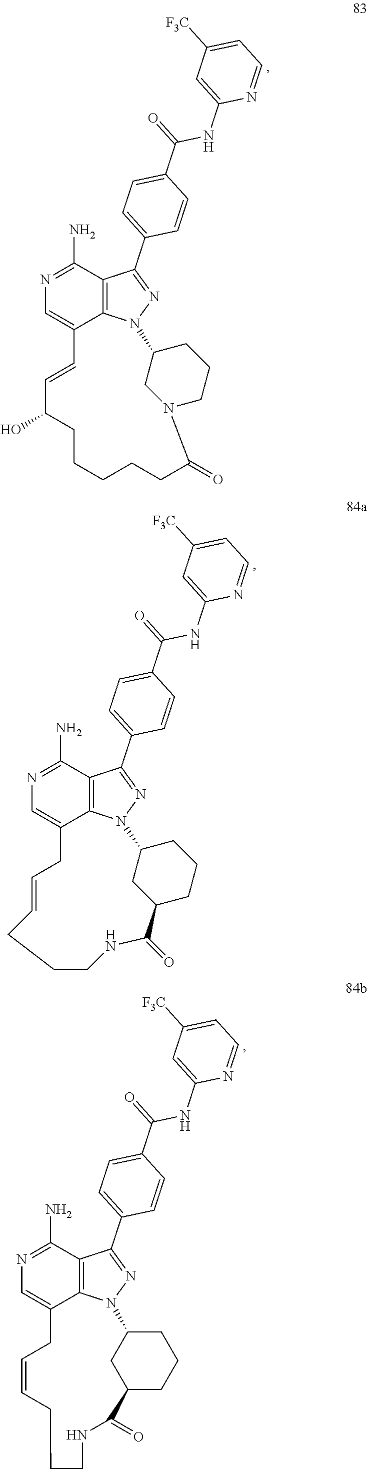

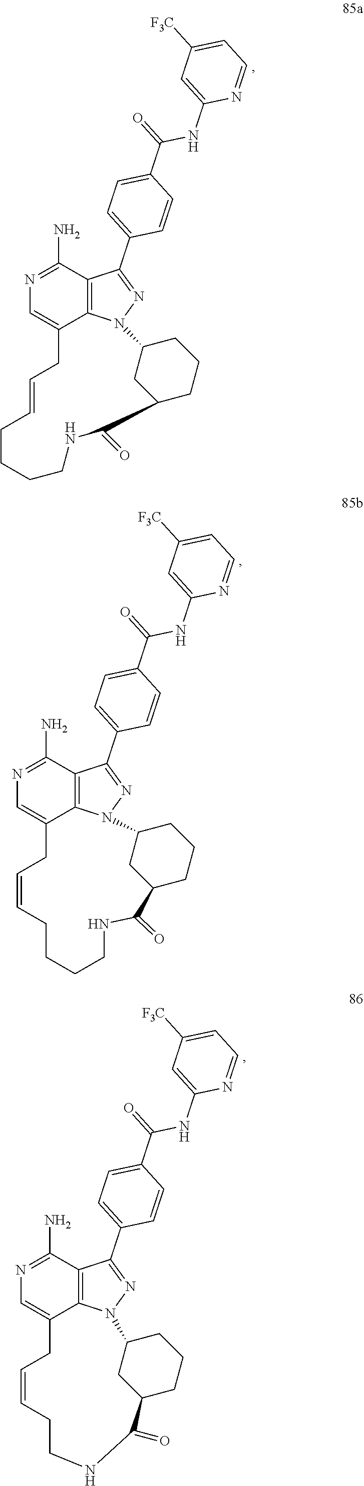 [SMILES: Nc1ncc2c3c1c(-c1ccc(C(=O)Nc4cc(C(F)(F)F)ccn4)cc1)nn3[C@@H]1CCC[C@H](C1)C(=O)NCCCC/C=C/C2 ; Nc1ncc2c3c1c(-c1ccc(C(=O)Nc4cc(C(F)(F)F)ccn4)cc1)nn3[C@@H]1CCC[C@H](C1)C(=O)NCCCC/C=C\C2 ; Nc1ncc2c3c1c(-c1ccc(C(=O)Nc4cc(C(F)(F)F)ccn4)cc1)nn3[C@@H]1CCC[C@H](C1)C(=O)NCC/C=C\C2]

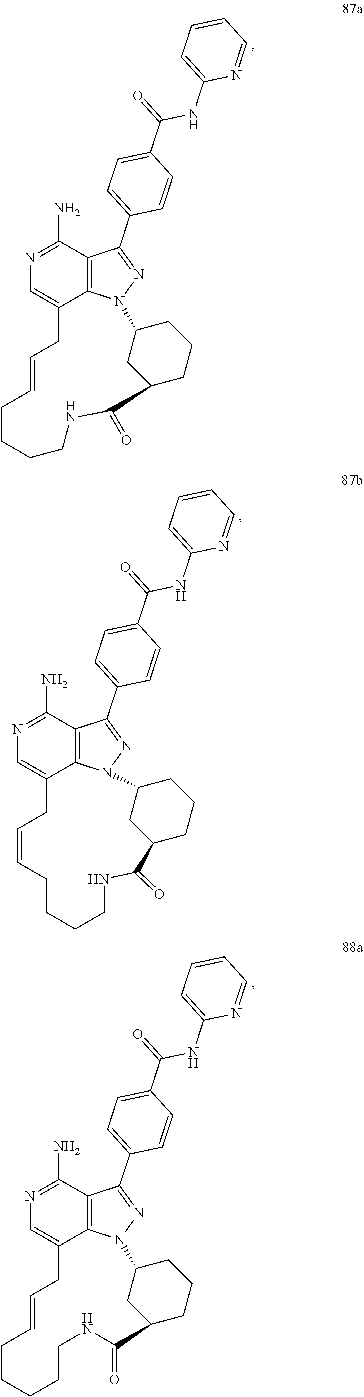 [SMILES: Nc1ncc2c3c1c(-c1ccc(C(=O)Nc4ccccn4)cc1)nn3[C@@H]1CCC[C@H](C1)C(=O)NCCCC/C=C/C2 ; Nc1ncc2c3c1c(-c1ccc(C(=O)Nc4ccccn4)cc1)nn3[C@@H]1CCC[C@H](C1)C(=O)NCCCC/C=C\C2 ; Nc1ncc2c3c1c(-c1ccc(C(=O)Nc4ccccn4)cc1)nn3[C@@H]1CCC[C@H](C1)C(=O)NCCCCC/C=C/C2]

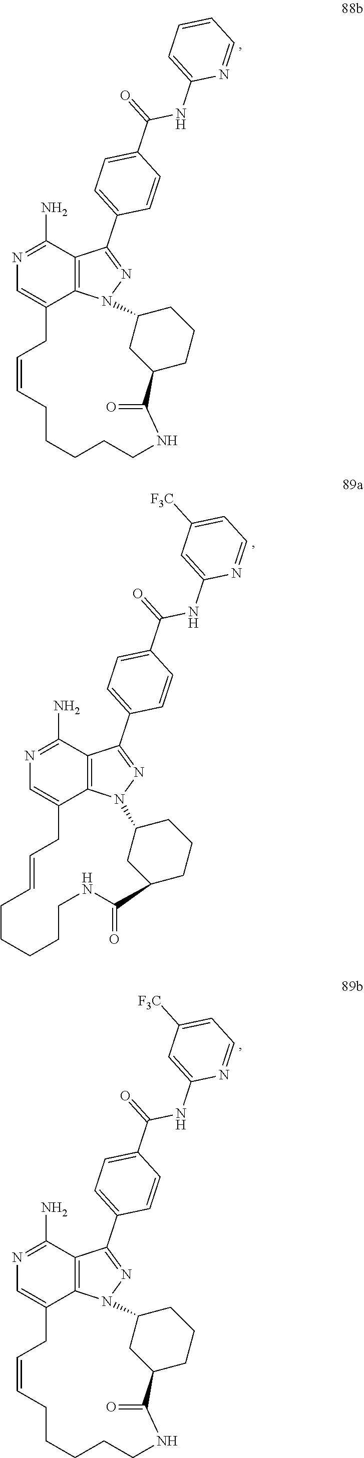 [SMILES: Nc1ncc2c3c1c(-c1ccc(C(=O)Nc4ccccn4)cc1)nn3[C@@H]1CCC[C@H](C1)C(=O)NCCCCC/C=C\C2 ; Nc1ncc2c3c1c(-c1ccc(C(=O)Nc4cc(C(F)(F)F)ccn4)cc1)nn3[C@@H]1CCC[C@H](C1)C(=O)NCCCCC/C=C/C2 ; Nc1ncc2c3c1c(-c1ccc(C(=O)Nc4cc(C(F)(F)F)ccn4)cc1)nn3[C@@H]1CCC[C@H](C1)C(=O)NCCCCC/C=C\C2]

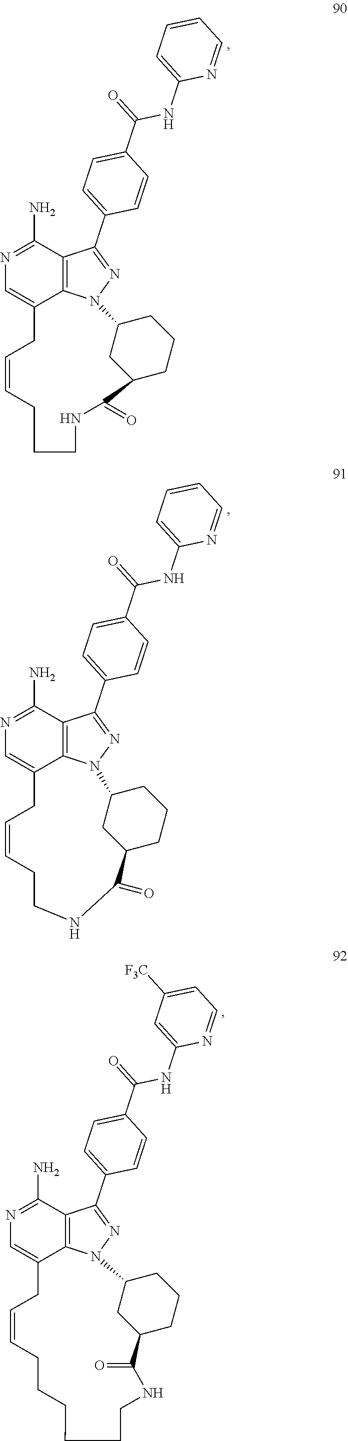 [SMILES: Nc1ncc2c3c1c(-c1ccc(C(=O)Nc4ccccn4)cc1)nn3[C@@H]1CCC[C@H](C1)C(=O)NCCC/C=C\C2 ; Nc1ncc2c3c1c(-c1ccc(C(=O)Nc4ccccn4)cc1)nn3[C@@H]1CCC[C@H](C1)C(=O)NCC/C=C\C2 ; Nc1ncc2c3c1c(-c1ccc(C(=O)Nc4cc(C(F)(F)F)ccn4)cc1)nn3[C@@H]1CCC[C@H](C1)C(=O)NCCCCCC/C=C\C2]

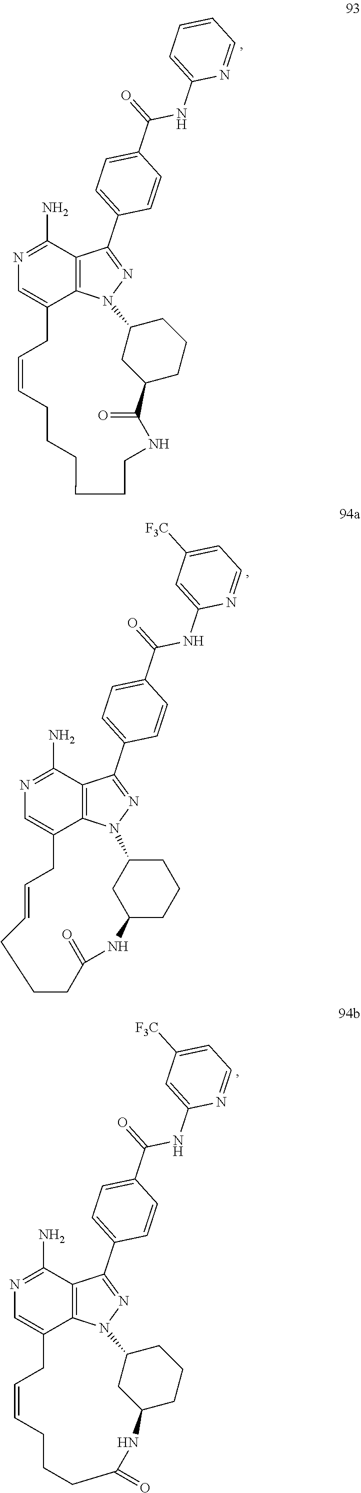 [SMILES: Nc1ncc2c3c1c(-c1ccc(C(=O)Nc4ccccn4)cc1)nn3[C@@H]1CCC[C@H](C1)C(=O)NCCCCCC/C=C\C2 ; Nc1ncc2c3c1c(-c1ccc(C(=O)Nc4cc(C(F)(F)F)ccn4)cc1)nn3[C@@H]1CCC[C@H](C1)NC(=O)CCC/C=C/C2 ; Nc1ncc2c3c1c(-c1ccc(C(=O)Nc4cc(C(F)(F)F)ccn4)cc1)nn3[C@@H]1CCC[C@H](C1)NC(=O)CCC/C=C\C2]

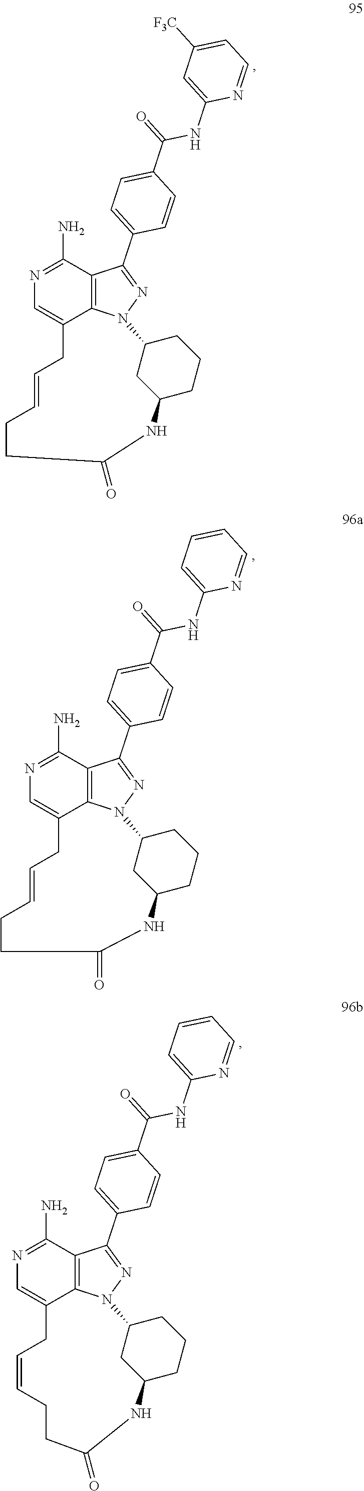 [SMILES: Nc1ncc2c3c1c(-c1ccc(C(=O)Nc4cc(C(F)(F)F)ccn4)cc1)nn3[C@@H]1CCC[C@H](C1)NC(=O)CC/C=C/C2 ; Nc1ncc2c3c1c(-c1ccc(C(=O)Nc4ccccn4)cc1)nn3[C@@H]1CCC[C@H](C1)NC(=O)CC/C=C/C2 ; Nc1ncc2c3c1c(-c1ccc(C(=O)Nc4ccccn4)cc1)nn3[C@@H]1CCC[C@H](C1)NC(=O)CC/C=C\C2]

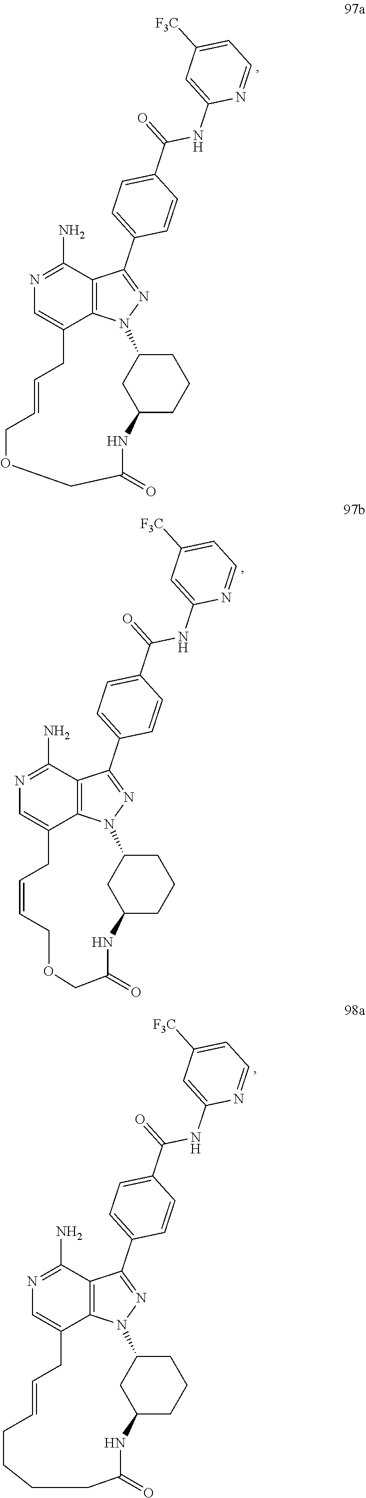

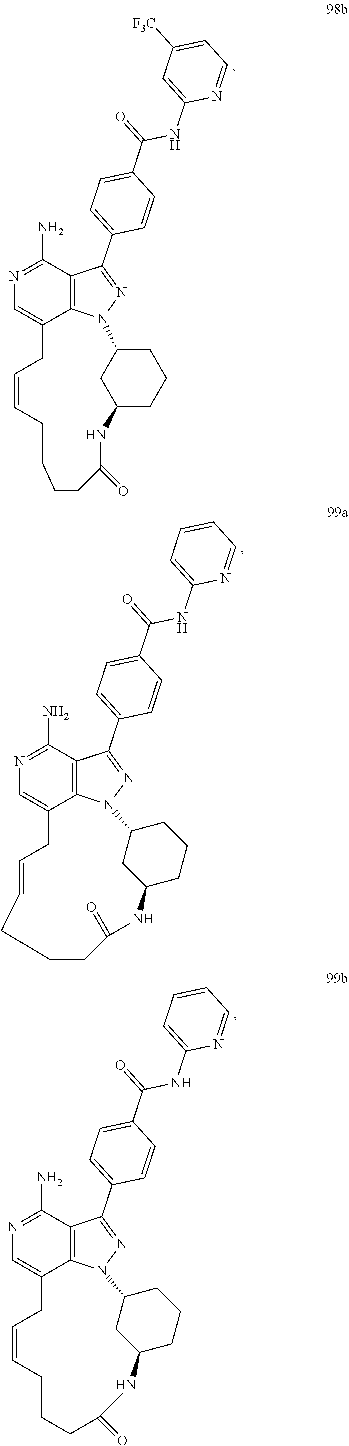 [SMILES: Nc1ncc2c3c1c(-c1ccc(C(=O)Nc4cc(C(F)(F)F)ccn4)cc1)nn3[C@@H]1CCC[C@H](C1)NC(=O)CCCC/C=C\C2 ; Nc1ncc2c3c1c(-c1ccc(C(=O)Nc4ccccn4)cc1)nn3[C@@H]1CCC[C@H](C1)NC(=O)CCC/C=C/C2 ; Nc1ncc2c3c1c(-c1ccc(C(=O)Nc4ccccn4)cc1)nn3[C@@H]1CCC[C@H](C1)NC(=O)CCC/C=C\C2]

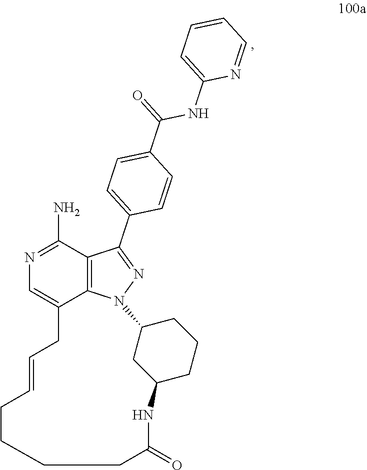 [SMILES: Nc1ncc2c3c1c(-c1ccc(C(=O)Nc4ccccn4)cc1)nn3[C@@H]1CCC[C@H](C1)NC(=O)CCCC/C=C/C2]

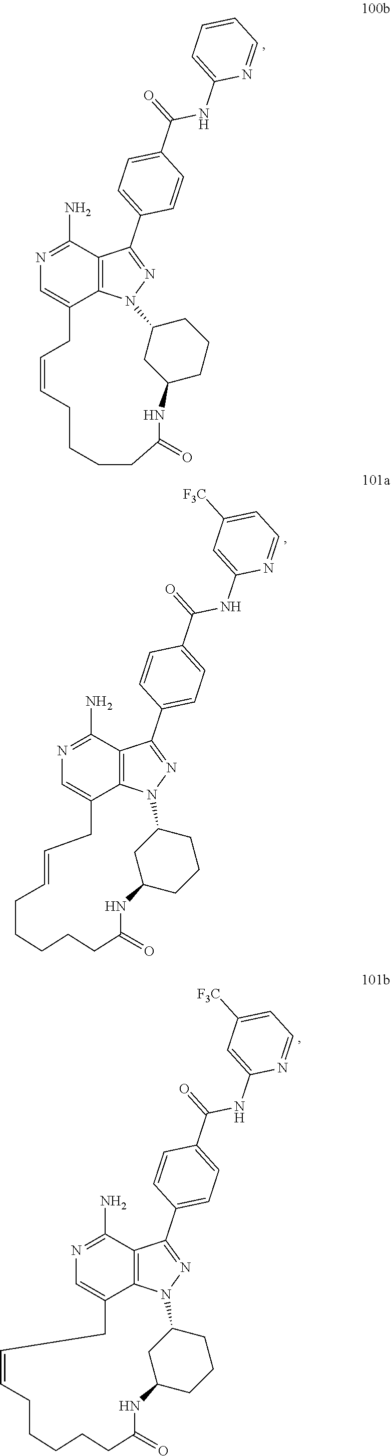

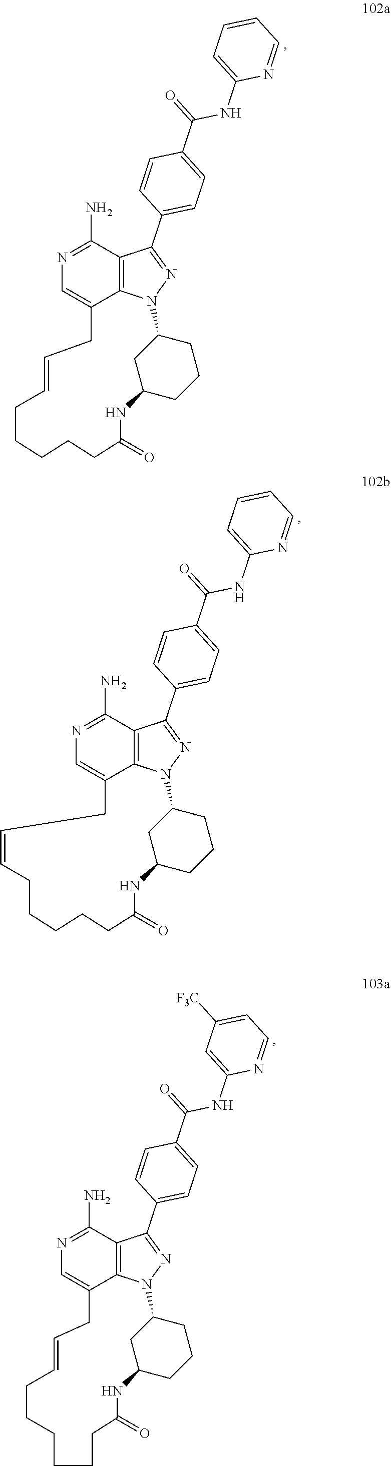 [SMILES: Nc1ncc2c3c1c(-c1ccc(C(=O)Nc4ccccn4)cc1)nn3[C@@H]1CCC[C@H](C1)NC(=O)CCCCC/C=C/C2 ; Nc1ncc2c3c1c(-c1ccc(C(=O)Nc4ccccn4)cc1)nn3[C@@H]1CCC[C@H](C1)NC(=O)CCCCC/C=C\C2 ; Nc1ncc2c3c1c(-c1ccc(C(=O)Nc4cc(C(F)(F)F)ccn4)cc1)nn3[C@@H]1CCC[C@H](C1)NC(=O)CCCCCC/C=C/C2]

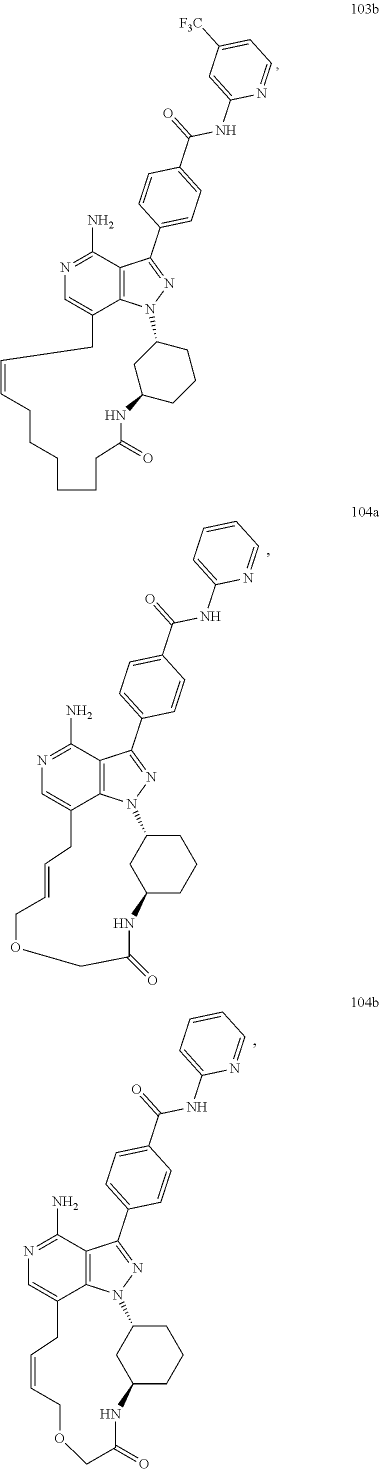 [SMILES: Nc1ncc2c3c1c(-c1ccc(C(=O)Nc4cc(C(F)(F)F)ccn4)cc1)nn3[C@@H]1CCC[C@H](C1)NC(=O)CCCCCC/C=C\C2 ; Nc1ncc2c3c1c(-c1ccc(C(=O)Nc4ccccn4)cc1)nn3[C@@H]1CCC[C@H](C1)NC(=O)COC/C=C/C2 ; Nc1ncc2c3c1c(-c1ccc(C(=O)Nc4ccccn4)cc1)nn3[C@@H]1CCC[C@H](C1)NC(=O)COC/C=C\C2]

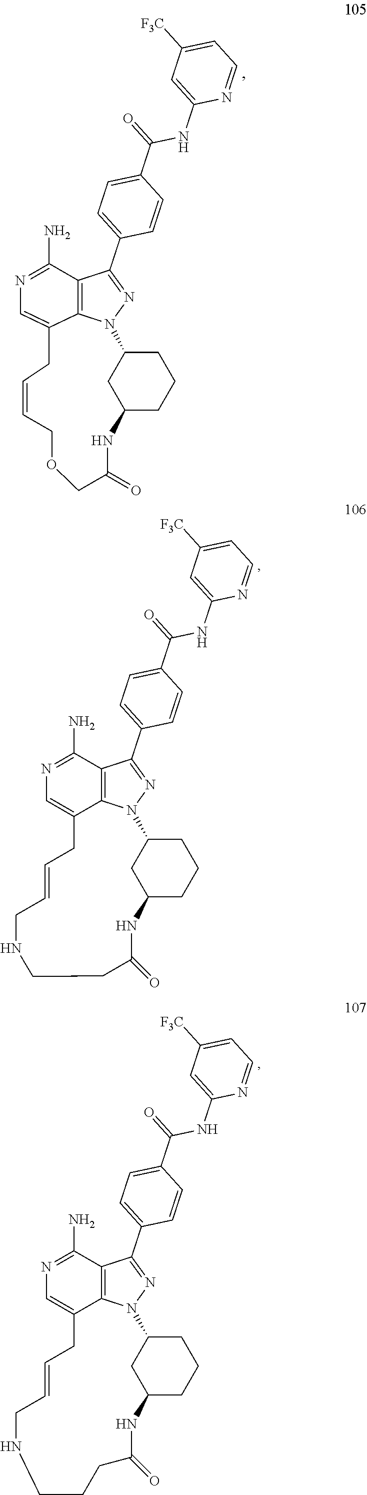

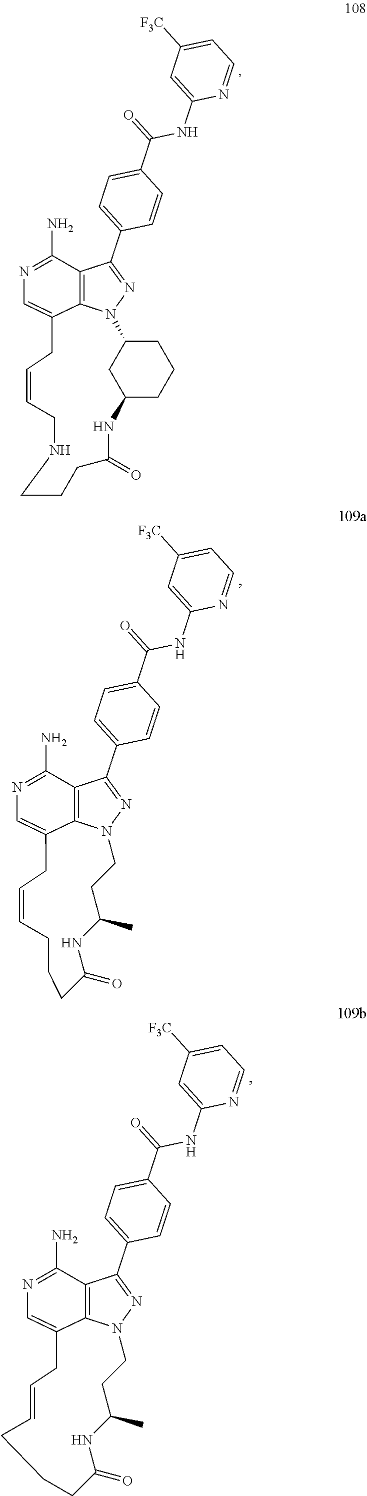 [SMILES: Nc1ncc2c3c1c(-c1ccc(C(=O)Nc4cc(C(F)(F)F)ccn4)cc1)nn3[C@@H]1CCC[C@H](C1)NC(=O)CCCNC/C=C\C2 ; C[C@@H]1CCn2nc(-c3ccc(C(=O)Nc4cc(C(F)(F)F)ccn4)cc3)c3c(N)ncc(c32)C/C=C\CCCC(=O)N1 ; C[C@@H]1CCn2nc(-c3ccc(C(=O)Nc4cc(C(F)(F)F)ccn4)cc3)c3c(N)ncc(c32)C/C=C/CCCC(=O)N1]

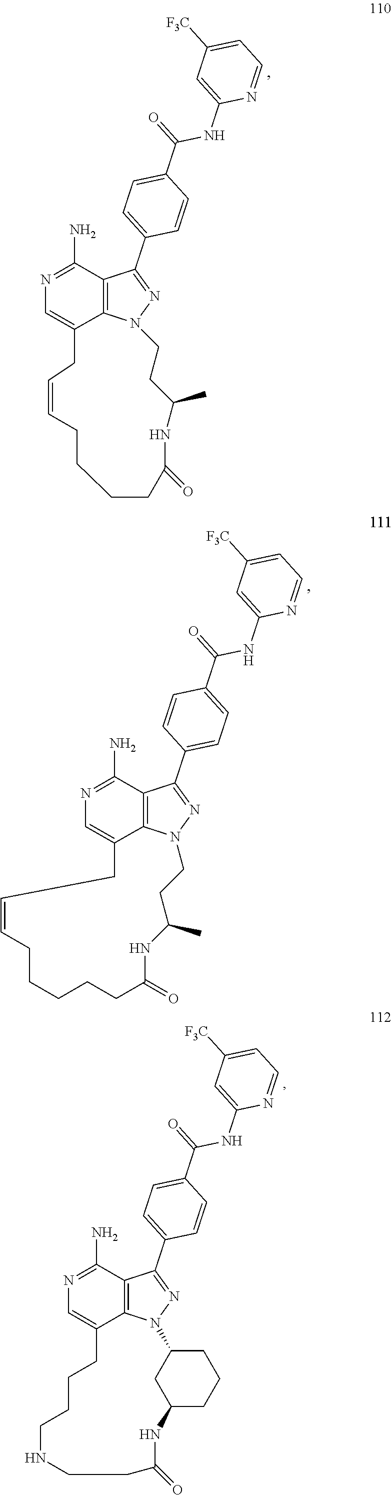

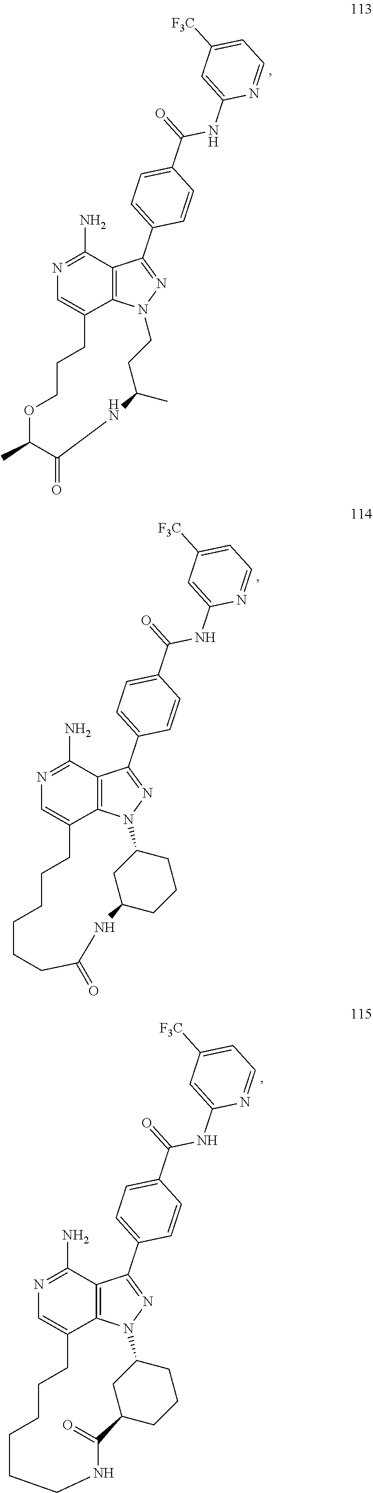 [SMILES: C[C@@H]1CCn2nc(-c3ccc(C(=O)Nc4cc(C(F)(F)F)ccn4)cc3)c3c(N)ncc(c32)CCCO[C@H](C)C(=O)N1 ; Nc1ncc2c3c1c(-c1ccc(C(=O)Nc4cc(C(F)(F)F)ccn4)cc1)nn3[C@@H]1CCC[C@H](C1)NC(=O)CCCCCC2 ; Nc1ncc2c3c1c(-c1ccc(C(=O)Nc4cc(C(F)(F)F)ccn4)cc1)nn3[C@@H]1CCC[C@H](C1)C(=O)NCCCCCC2]

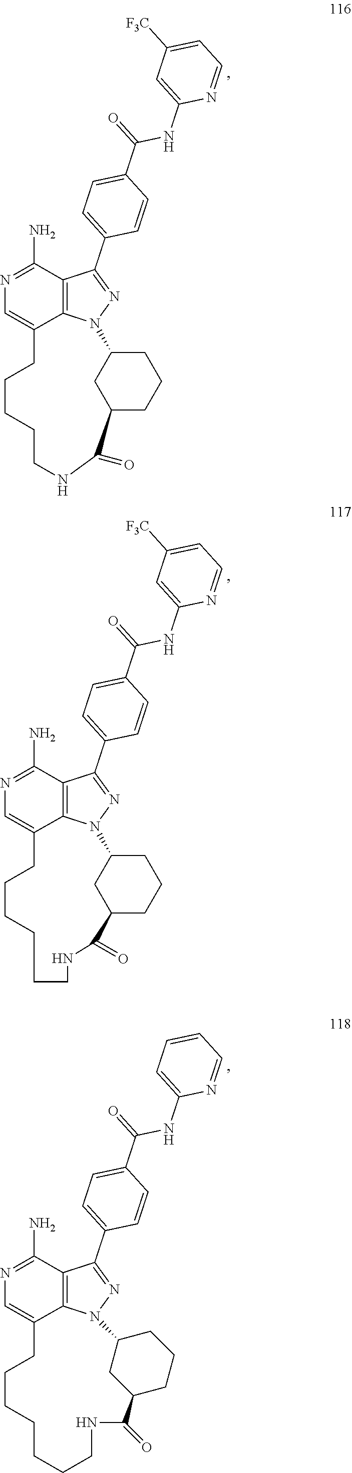 [SMILES: Nc1ncc2c3c1c(-c1ccc(C(=O)Nc4cc(C(F)(F)F)ccn4)cc1)nn3[C@@H]1CCC[C@H](C1)C(=O)NCCCCC2 ; Nc1ncc2c3c1c(-c1ccc(C(=O)Nc4cc(C(F)(F)F)ccn4)cc1)nn3[C@@H]1CCC[C@H](C1)C(=O)NCCCCCC2 ; Nc1ncc2c3c1c(-c1ccc(C(=O)Nc4ccccn4)cc1)nn3[C@@H]1CCC[C@H](C1)C(=O)NCCCCCCC2]

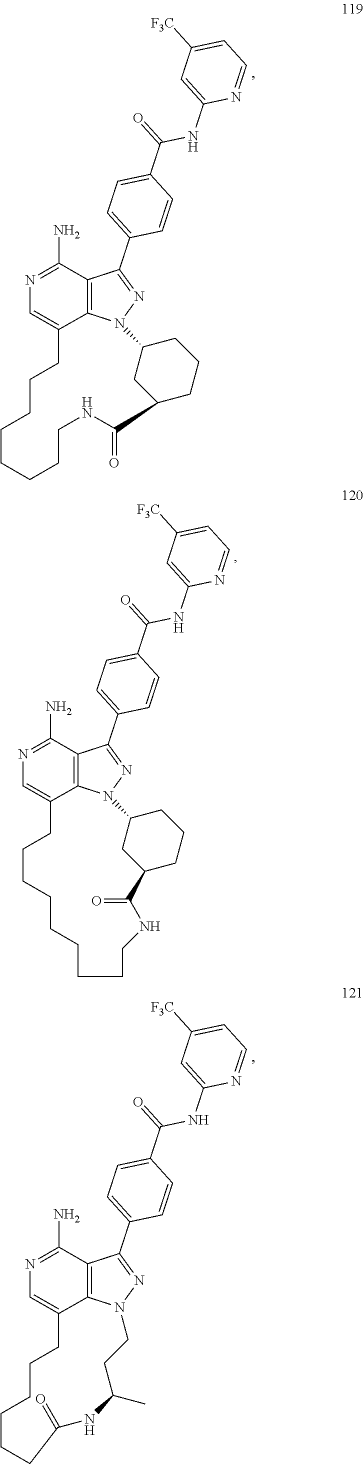 [SMILES: Nc1ncc2c3c1c(-c1ccc(C(=O)Nc4cc(C(F)(F)F)ccn4)cc1)nn3[C@@H]1CCC[C@H](C1)C(=O)NCCCCCCCC2 ; Nc1ncc2c3c1c(-c1ccc(C(=O)Nc4cc(C(F)(F)F)ccn4)cc1)nn3[C@@H]1CCC[C@H](C1)C(=O)NCCCCCCCCC2 ; C[C@@H]1CCn2nc(-c3ccc(C(=O)Nc4cc(C(F)(F)F)ccn4)cc3)c3c(N)ncc(c32)CCCCCCC(=O)N1]

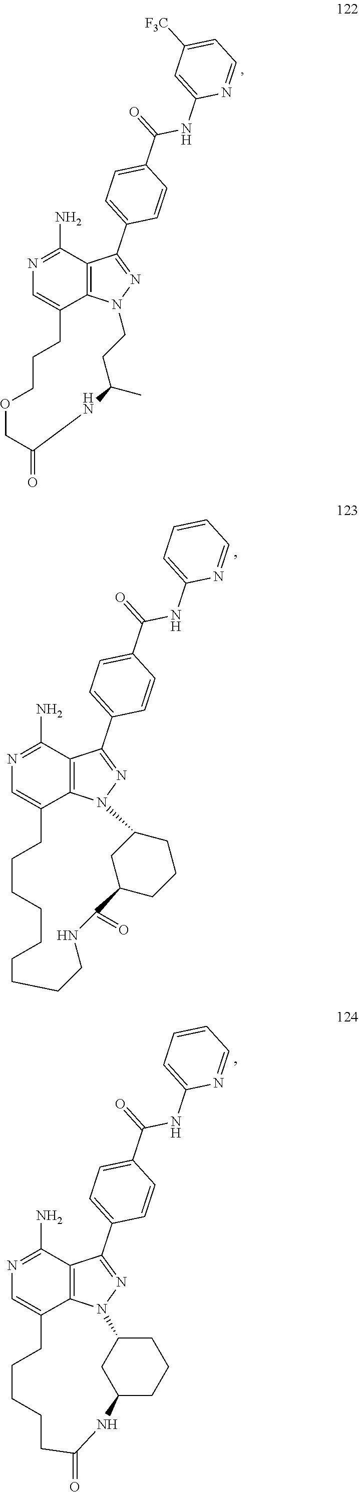 [SMILES: C[C@@H]1CCn2nc(-c3ccc(C(=O)Nc4cc(C(F)(F)F)ccn4)cc3)c3c(N)ncc(c32)CCCOCC(=O)N1 ; Nc1ncc2c3c1c(-c1ccc(C(=O)Nc4ccccn4)cc1)nn3[C@@H]1CCC[C@H](C1)C(=O)NCCCCCCCCC2 ; Nc1ncc2c3c1c(-c1ccc(C(=O)Nc4ccccn4)cc1)nn3[C@@H]1CCC[C@H](C1)NC(=O)CCCCC2]

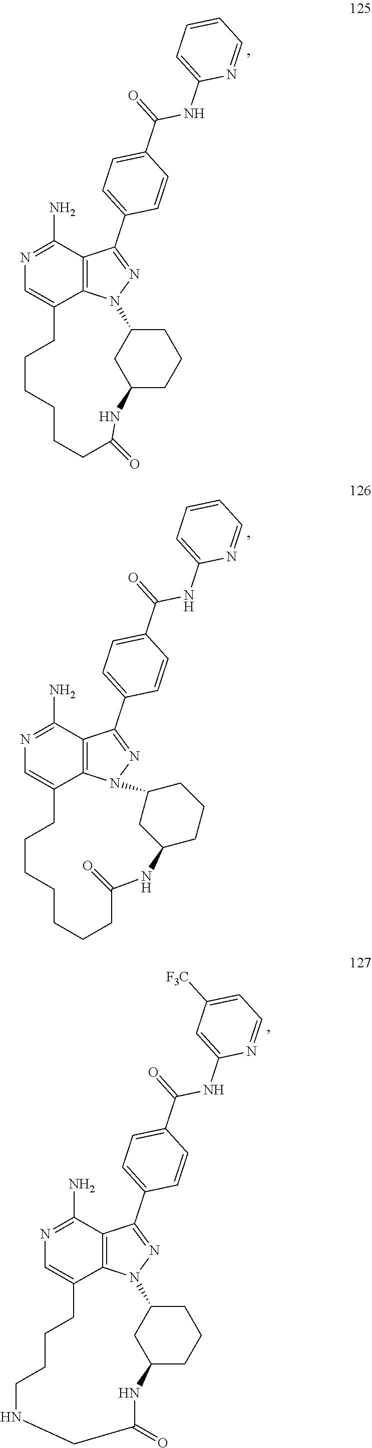 [SMILES: Nc1ncc2c3c1c(-c1ccc(C(=O)Nc4ccccn4)cc1)nn3[C@@H]1CCC[C@H](C1)NC(=O)CCCCCC2 ; Nc1ncc2c3c1c(-c1ccc(C(=O)Nc4ccccn4)cc1)nn3[C@@H]1CCC[C@H](C1)NC(=O)CCCCCCC2 ; Nc1ncc2c3c1c(-c1ccc(C(=O)Nc4cc(C(F)(F)F)ccn4)cc1)nn3[C@@H]1CCC[C@H](C1)NC(=O)CNCCCC2]

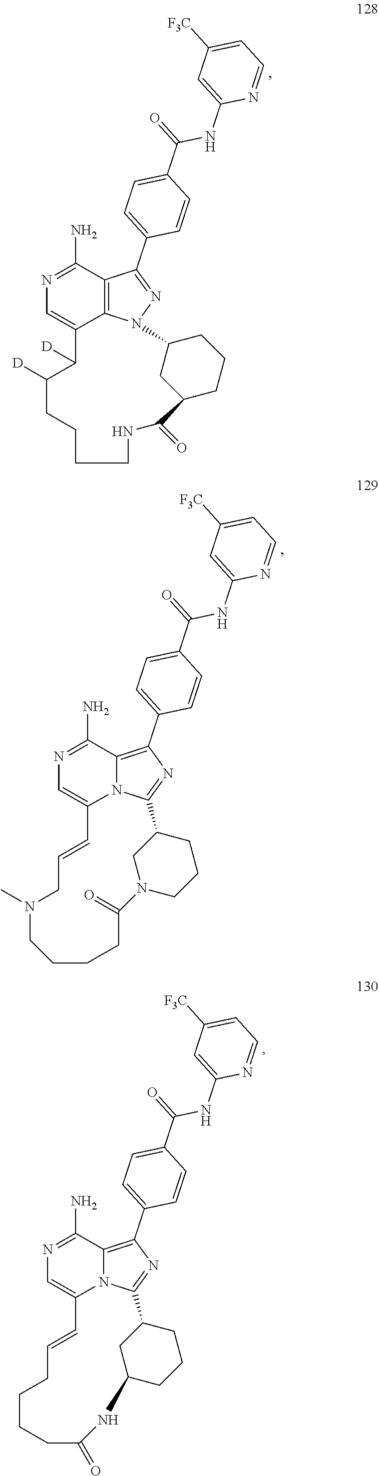 [SMILES: [2H]C1CCCCNC(=O)[C@@H]2CCC[C@H](C2)n2nc(-c3ccc(C(=O)Nc4cc(C(F)(F)F)ccn4)cc3)c3c(N)ncc(c32)C1[2H] ; CN1C/C=C/c2cnc(N)c3c(-c4ccc(C(=O)Nc5cc(C(F)(F)F)ccn5)cc4)nc(n23)[C@@H]2CCCN(C2)C(=O)CCCC1 ; Nc1ncc2n3c(nc(-c4ccc(C(=O)Nc5cc(C(F)(F)F)ccn5)cc4)c13)[C@@H]1CCC[C@H](C1)NC(=O)CCCC/C=C/2]

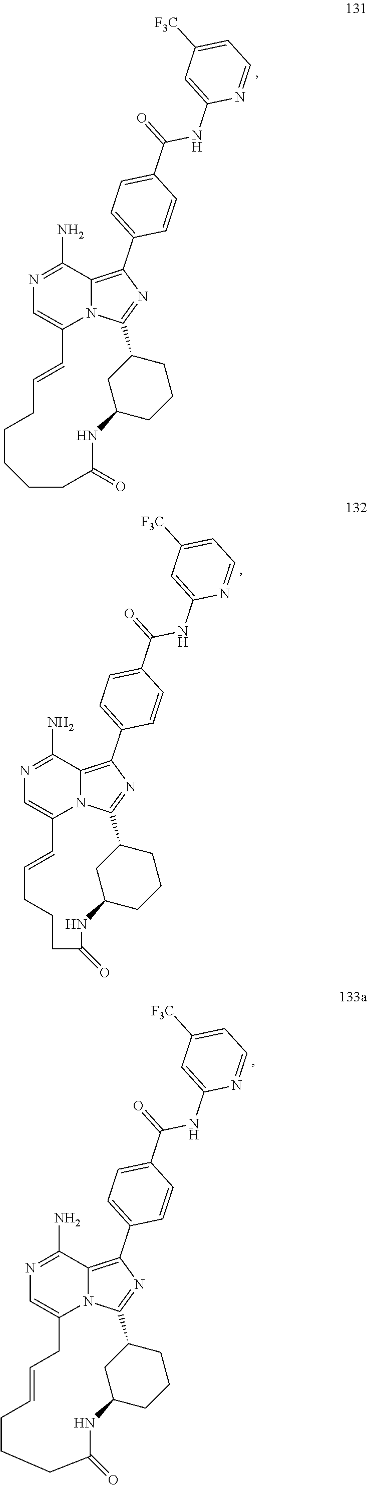 [SMILES: Nc1ncc2n3c(nc(-c4ccc(C(=O)Nc5cc(C(F)(F)F)ccn5)cc4)c13)[C@@H]1CCC[C@H](C1)NC(=O)CCCCC/C=C/2 ; Nc1ncc2n3c(nc(-c4ccc(C(=O)Nc5cc(C(F)(F)F)ccn5)cc4)c13)[C@@H]1CCC[C@H](C1)NC(=O)CCC/C=C/2 ; Nc1ncc2n3c(nc(-c4ccc(C(=O)Nc5cc(C(F)(F)F)ccn5)cc4)c13)[C@@H]1CCC[C@H](C1)NC(=O)CCC/C=C/C2]

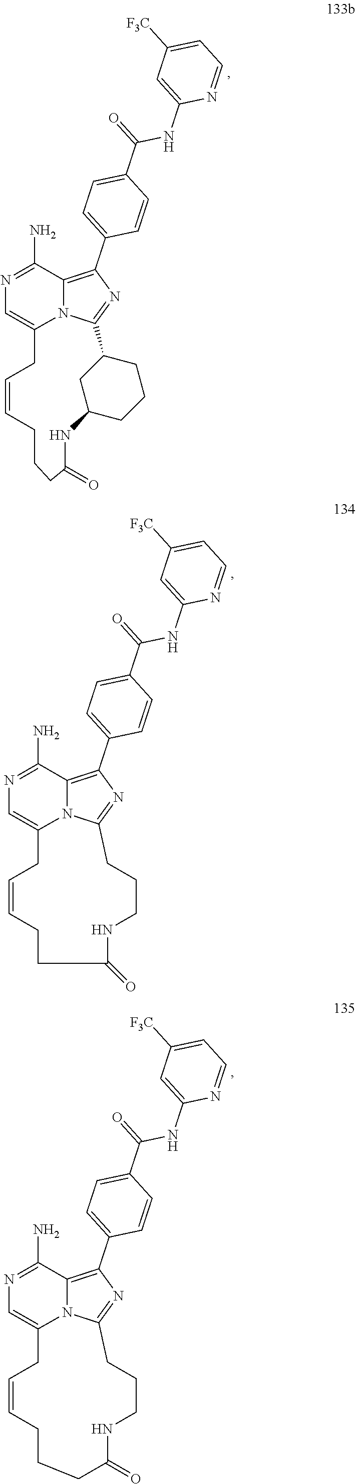

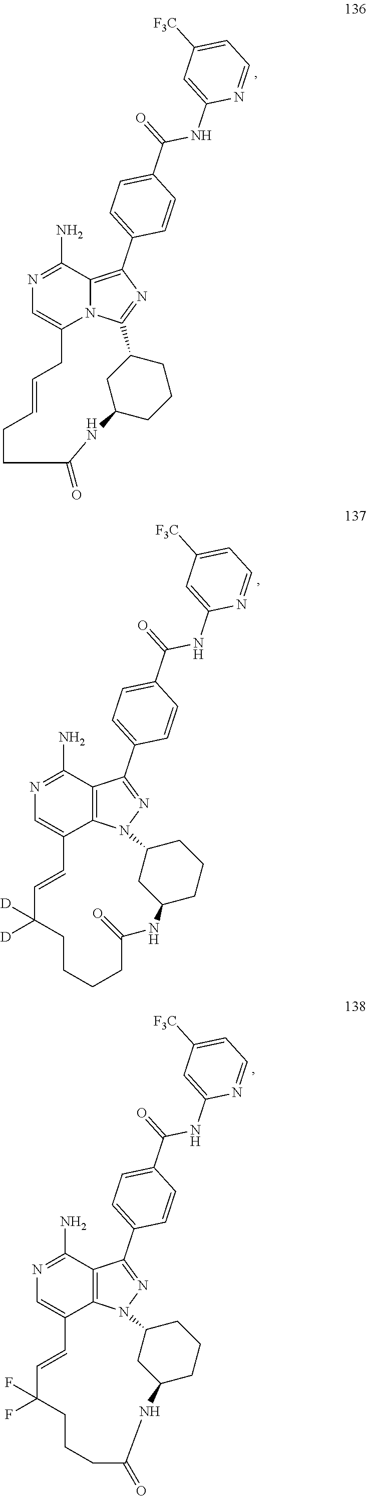 [SMILES: Nc1ncc2n3c(nc(-c4ccc(C(=O)Nc5cc(C(F)(F)F)ccn5)cc4)c13)[C@@H]1CCC[C@H](C1)NC(=O)CC/C=C/C2 ; [2H]C1([2H])/C=C/c2cnc(N)c3c(-c4ccc(C(=O)Nc5cc(C(F)(F)F)ccn5)cc4)nn(c23)[C@@H]2CCC[C@H](C2)NC(=O)CCCC1 ; Nc1ncc2c3c1c(-c1ccc(C(=O)Nc4cc(C(F)(F)F)ccn4)cc1)nn3[C@@H]1CCC[C@H](C1)NC(=O)CCCC(F)(F)/C=C/2]

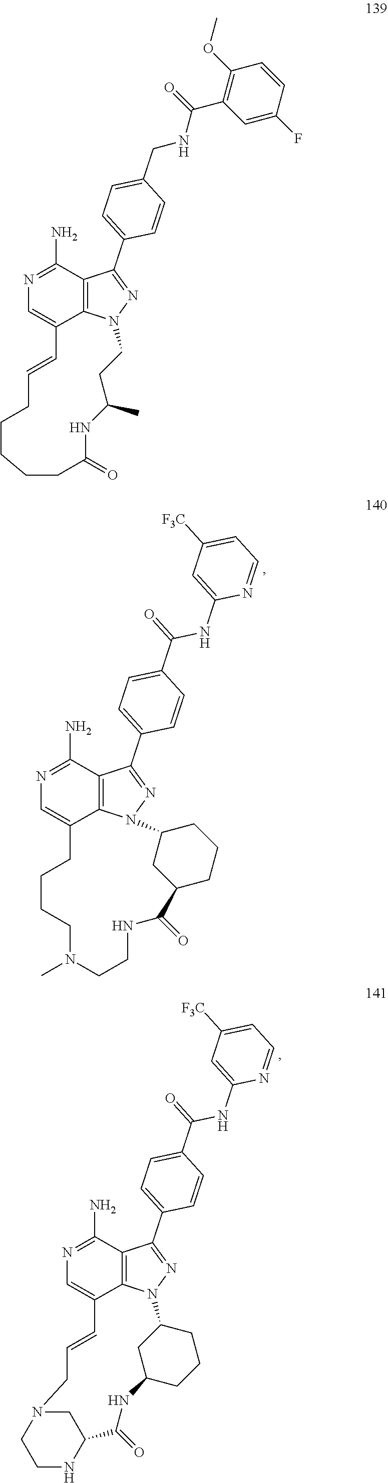

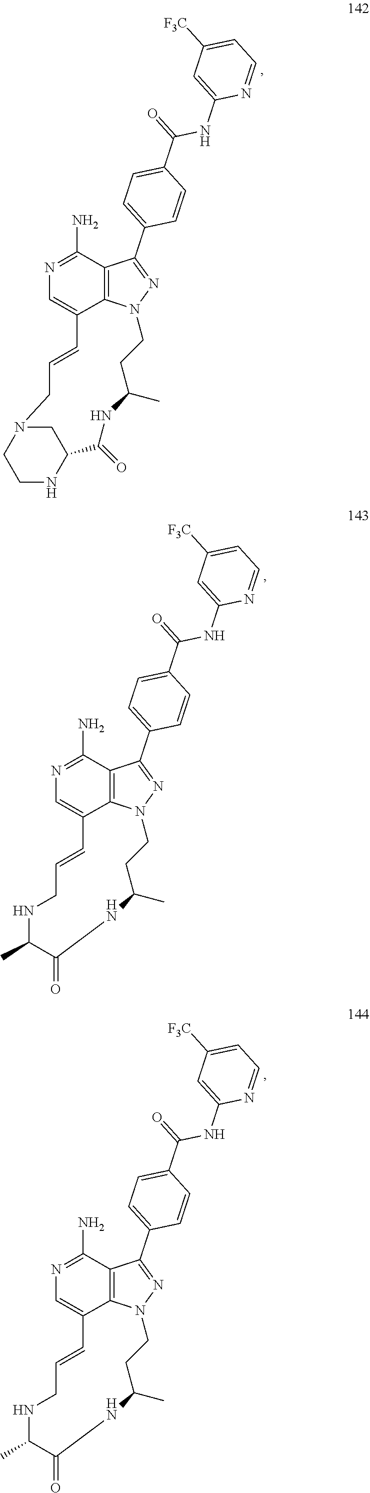 [SMILES: C[C@@H]1CCn2nc(-c3ccc(C(=O)Nc4cc(C(F)(F)F)ccn4)cc3)c3c(N)ncc(c32)/C=C/CN2CCN[C@H](C2)C(=O)N1 ; C[C@@H]1CCn2nc(-c3ccc(C(=O)Nc4cc(C(F)(F)F)ccn4)cc3)c3c(N)ncc(c32)/C=C/CN[C@H](C)C(=O)N1 ; C[C@@H]1CCn2nc(-c3ccc(C(=O)Nc4cc(C(F)(F)F)ccn4)cc3)c3c(N)ncc(c32)/C=C/CN[C@@H](C)C(=O)N1]

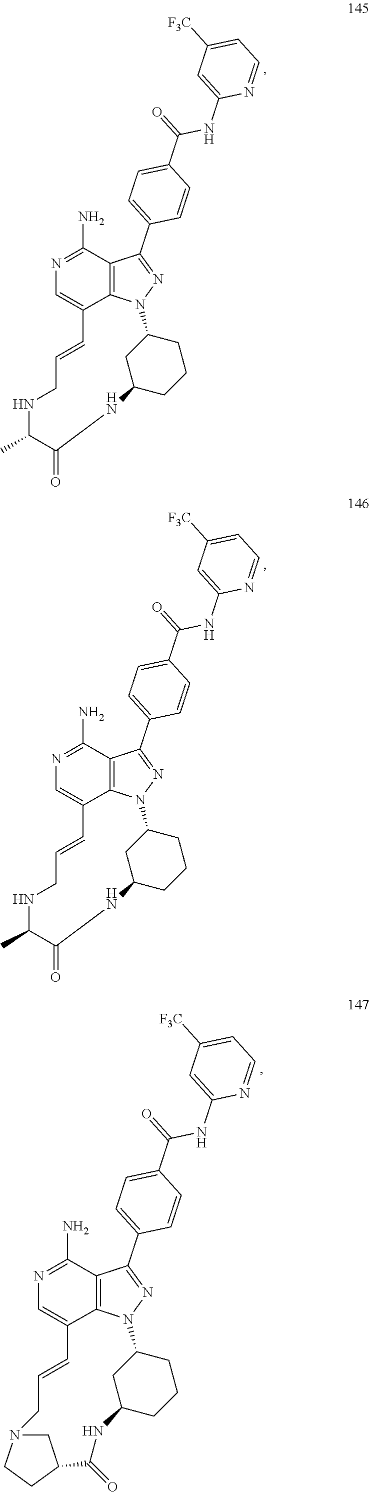 [SMILES: C[C@@H]1NC/C=C/c2cnc(N)c3c(-c4ccc(C(=O)Nc5cc(C(F)(F)F)ccn5)cc4)nn(c23)[C@@H]2CCC[C@H](C2)NC1=O ; C[C@H]1NC/C=C/c2cnc(N)c3c(-c4ccc(C(=O)Nc5cc(C(F)(F)F)ccn5)cc4)nn(c23)[C@@H]2CCC[C@H](C2)NC1=O ; Nc1ncc2c3c1c(-c1ccc(C(=O)Nc4cc(C(F)(F)F)ccn4)cc1)nn3[C@@H]1CCC[C@H](C1)NC(=O)[C@@H]1CCN(C/C=C/2)C1]

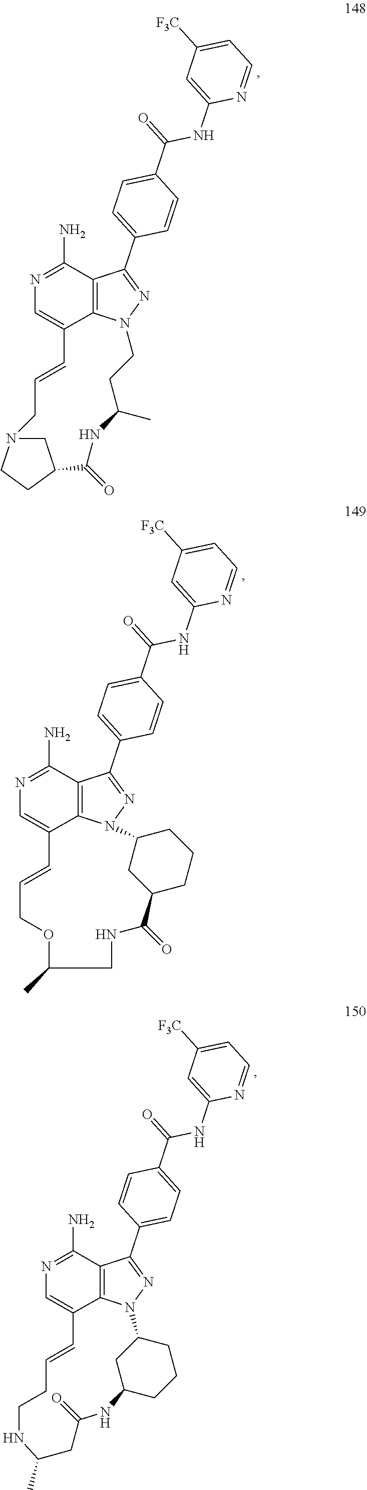 [SMILES: C[C@@H]1CCn2nc(-c3ccc(C(=O)Nc4cc(C(F)(F)F)ccn4)cc3)c3c(N)ncc(c32)/C=C/CN2CC[C@H](C2)C(=O)N1 ; C[C@@H]1CNC(=O)[C@@H]2CCC[C@H](C2)n2nc(-c3ccc(C(=O)Nc4cc(C(F)(F)F)ccn4)cc3)c3c(N)ncc(c32)/C=C/CO1 ; C[C@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCN1]

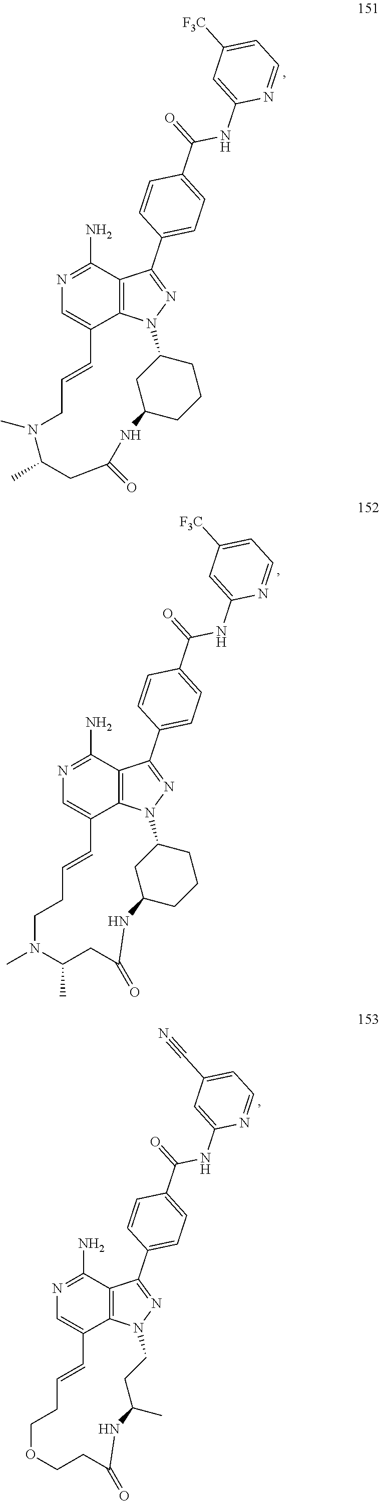

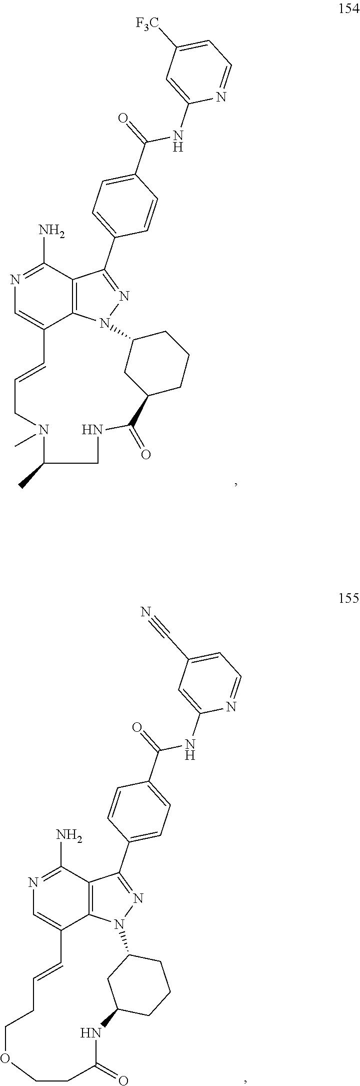 [SMILES: C[C@@H]1CNC(=O)[C@@H]2CCC[C@H](C2)n2nc(-c3ccc(C(=O)Nc4cc(C(F)(F)F)ccn4)cc3)c3c(N)ncc(c32)/C=C/CN1C ; N#Cc1ccnc(NC(=O)c2ccc(-c3nn4c5c(cnc(N)c35)/C=C/CCOCCC(=O)N[C@@H]3CCC[C@@H]4C3)cc2)c1]

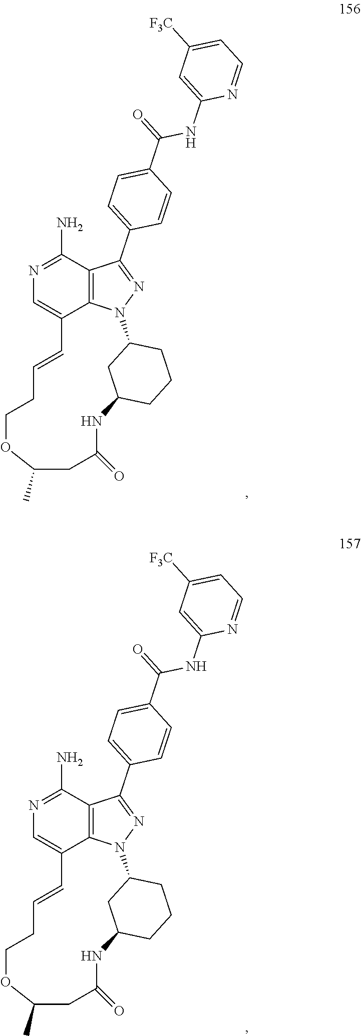 [SMILES: C[C@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCO1 ; C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCO1]

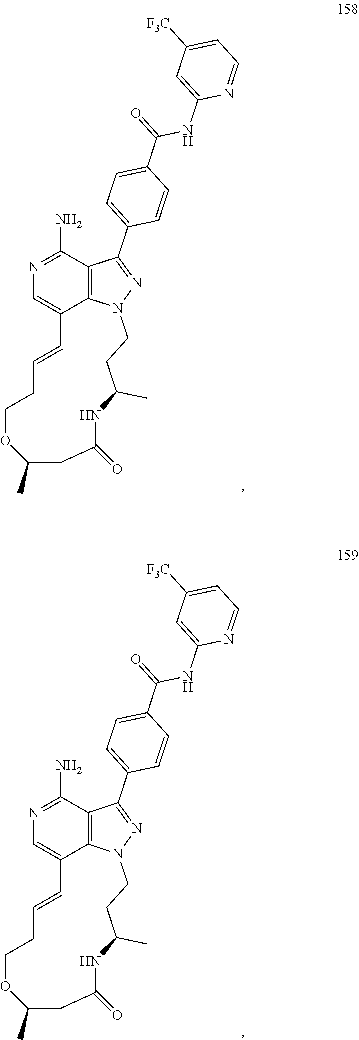 [SMILES: C[C@@H]1CCn2nc(-c3ccc(C(=O)Nc4cc(C(F)(F)F)ccn4)cc3)c3c(N)ncc(c32)/C=C/CCO[C@H](C)CC(=O)N1 ; C[C@@H]1CCn2nc(-c3ccc(C(=O)Nc4cc(C(F)(F)F)ccn4)cc3)c3c(N)ncc(c32)/C=C/CCO[C@H](C)CC(=O)N1]

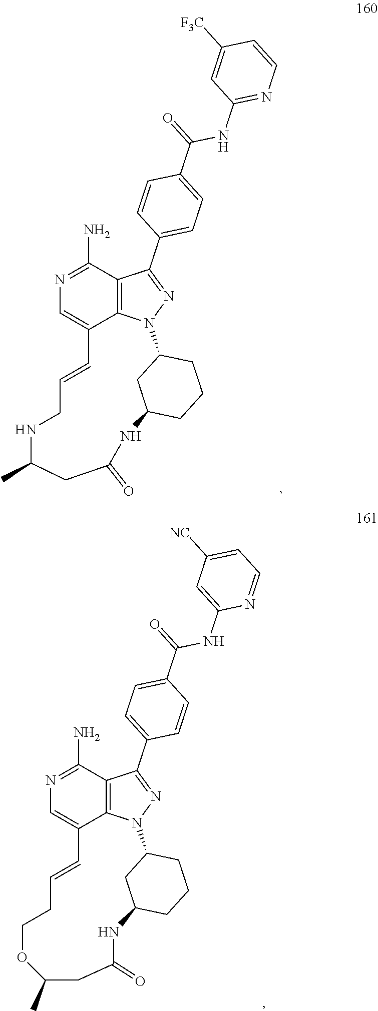 [SMILES: C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CN1 ; C[C@@H]1CC(=O)N[C@@H]2CCC[C@H](C2)n2nc(-c3ccc(C(=O)Nc4cc(C#N)ccn4)cc3)c3c(N)ncc(c32)/C=C/CCO1]

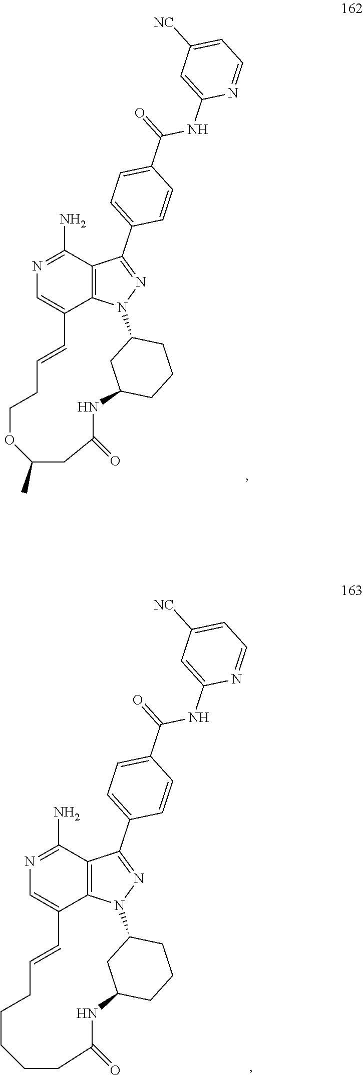 [SMILES: C[C@@H]1CC(=O)N[C@@H]2CCC[C@H](C2)n2nc(-c3ccc(C(=O)Nc4cc(C#N)ccn4)cc3)c3c(N)ncc(c32)/C=C/CCO1 ; N#Cc1ccnc(NC(=O)c2ccc(-c3nn4c5c(cnc(N)c35)/C=C/CCCCCC(=O)N[C@@H]3CCC[C@@H]4C3)cc2)c1]

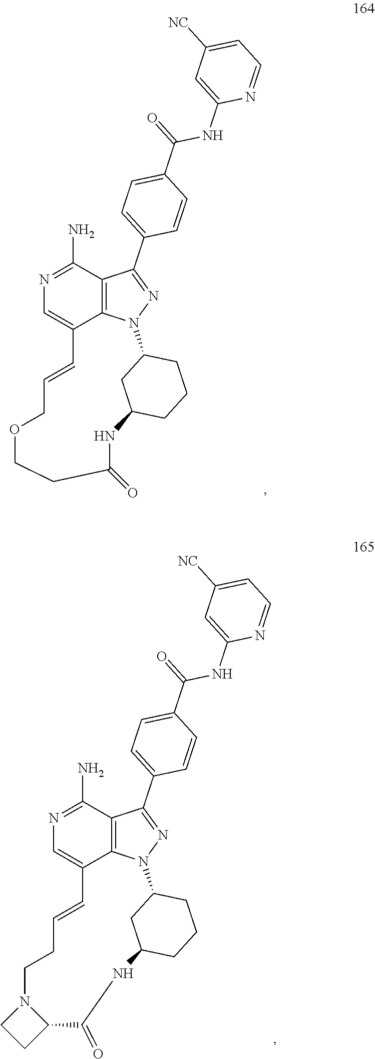 [SMILES: N#Cc1ccnc(NC(=O)c2ccc(-c3nn4c5c(cnc(N)c35)/C=C/COCCC(=O)N[C@@H]3CCC[C@@H]4C3)cc2)c1 ; N#Cc1ccnc(NC(=O)c2ccc(-c3nn4c5c(cnc(N)c35)/C=C/CCN3CC[C@H]3C(=O)N[C@@H]3CCC[C@@H]4C3)cc2)c1]

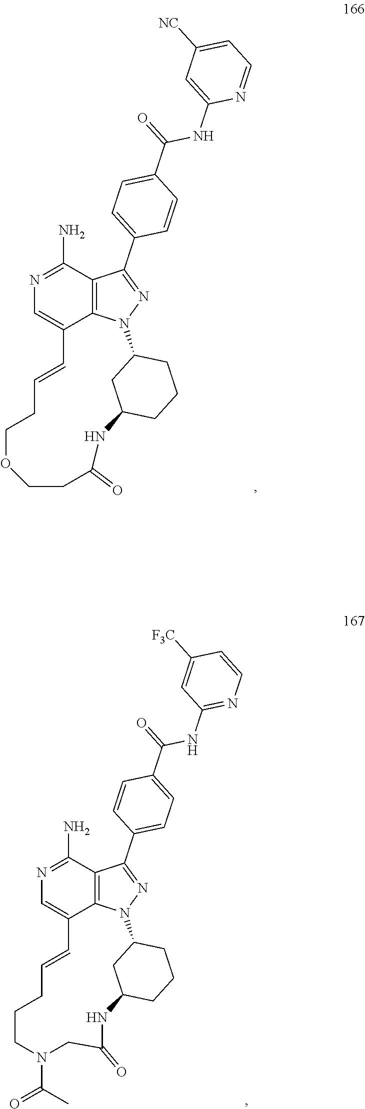 [SMILES: N#Cc1ccnc(NC(=O)c2ccc(-c3nn4c5c(cnc(N)c35)/C=C/CCOCCC(=O)N[C@@H]3CCC[C@@H]4C3)cc2)c1 ; CC(=O)N1CCC/C=C/c2cnc(N)c3c(-c4ccc(C(=O)Nc5cc(C(F)(F)F)ccn5)cc4)nn(c23)[C@@H]2CCC[C@H](C2)NC(=O)C1]

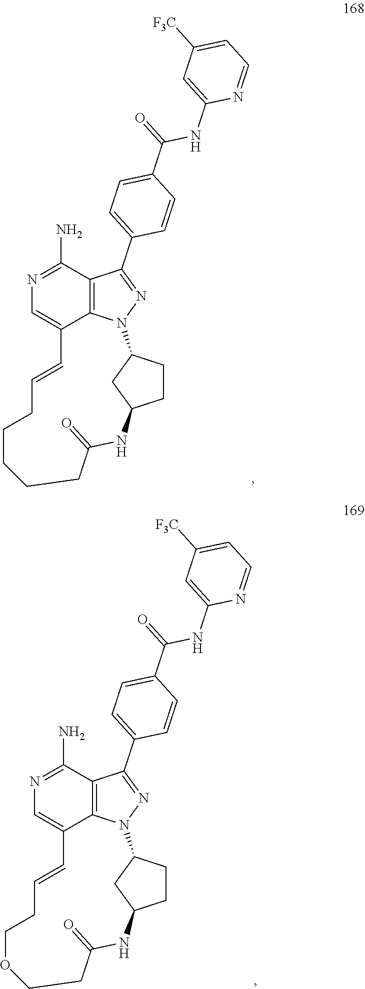 [SMILES: Nc1ncc2c3c1c(-c1ccc(C(=O)Nc4cc(C(F)(F)F)ccn4)cc1)nn3[C@@H]1CC[C@H](C1)NC(=O)CCCCC/C=C/2 ; Nc1ncc2c3c1c(-c1ccc(C(=O)Nc4cc(C(F)(F)F)ccn4)cc1)nn3[C@@H]1CC[C@H](C1)NC(=O)CCOCC/C=C/2]

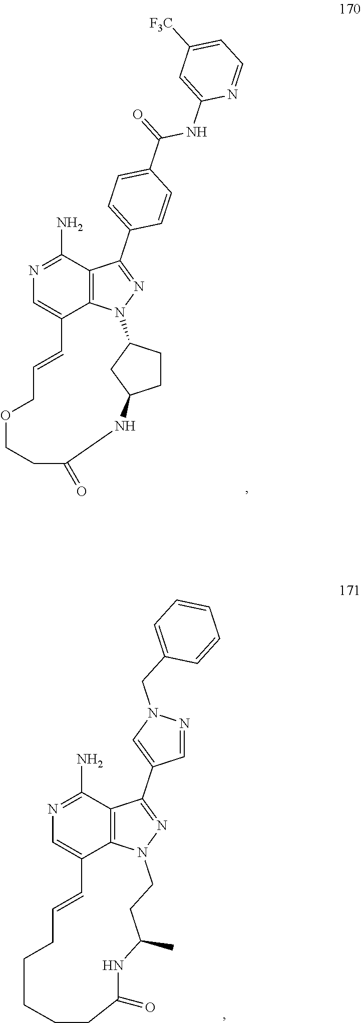 [SMILES: Nc1ncc2c3c1c(-c1ccc(C(=O)Nc4cc(C(F)(F)F)ccn4)cc1)nn3[C@@H]1CC[C@H](C1)NC(=O)CCOC/C=C/2 ; C[C@@H]1CCn2nc(-c3cnn(Cc4ccccc4)c3)c3c(N)ncc(c32)/C=C/CCCCCC(=O)N1]

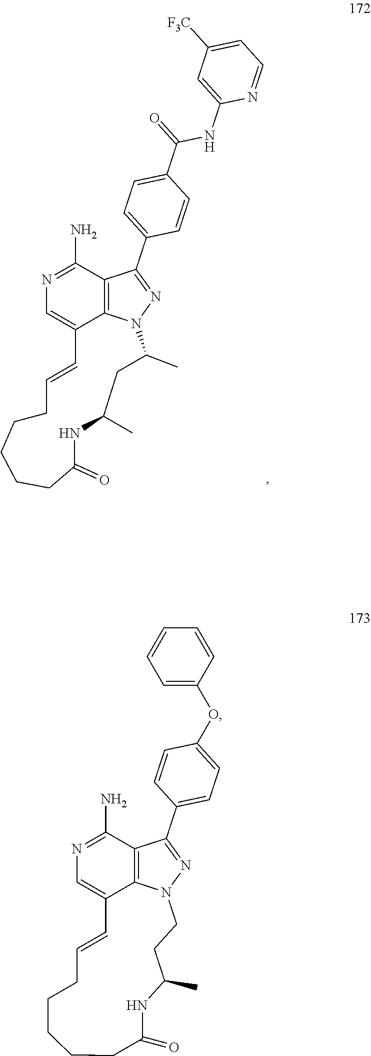 [SMILES: C[C@@H]1C[C@@H](C)n2nc(-c3ccc(C(=O)Nc4cc(C(F)(F)F)ccn4)cc3)c3c(N)ncc(c32)/C=C/CCCCCC(=O)N1 ; C[C@@H]1CCn2nc(-c3ccc(Oc4ccccc4)cc3)c3c(N)ncc(c32)/C=C/CCCCCC(=O)N1]

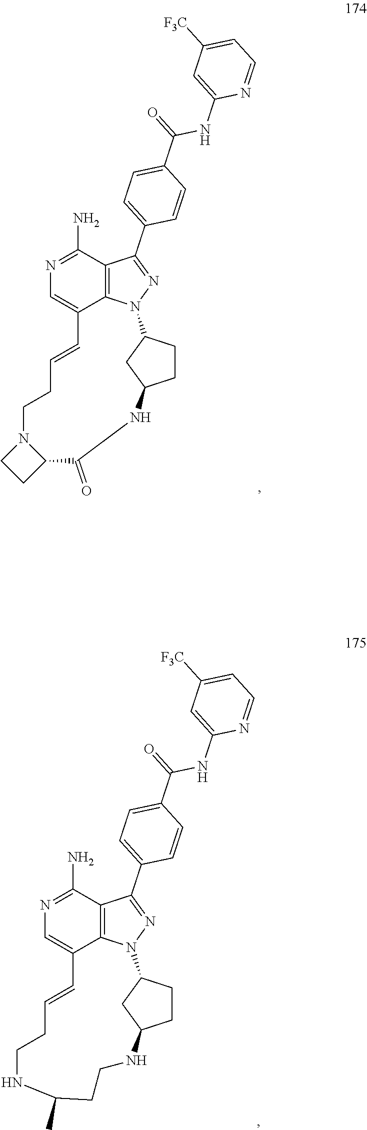

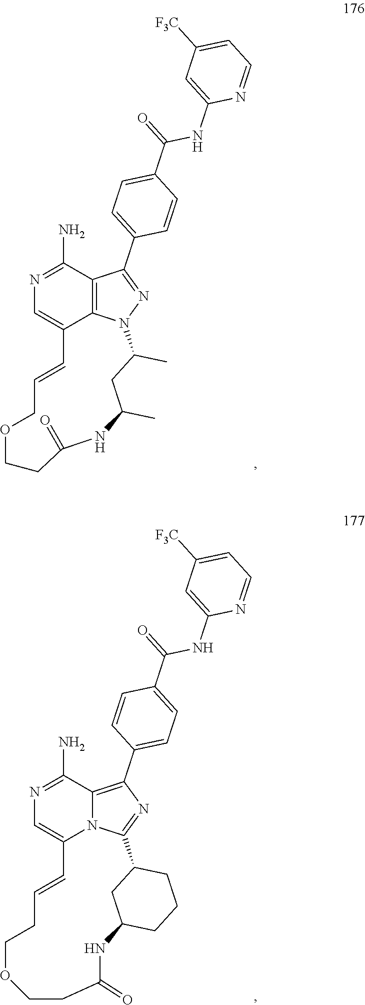 [SMILES: C[C@@H]1C[C@@H](C)n2nc(-c3ccc(C(=O)Nc4cc(C(F)(F)F)ccn4)cc3)c3c(N)ncc(c32)/C=C/COCCC(=O)N1 ; Nc1ncc2n3c(nc(-c4ccc(C(=O)Nc5cc(C(F)(F)F)ccn5)cc4)c13)[C@@H]1CCC[C@H](C1)NC(=O)CCOCC/C=C/2]

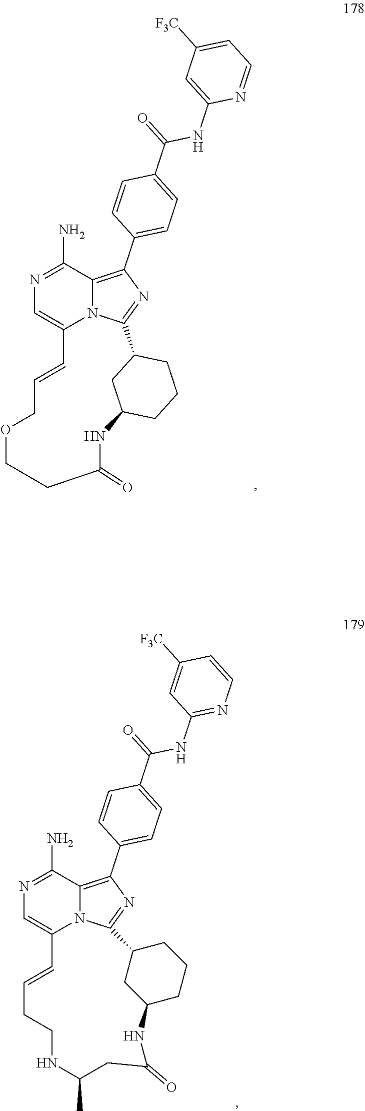 [SMILES: Nc1ncc2n3c(nc(-c4ccc(C(=O)Nc5cc(C(F)(F)F)ccn5)cc4)c13)[C@@H]1CCC[C@H](C1)NC(=O)CCOC/C=C/2 ; C[C@@H]1CC(=O)N[C@@H]2CCC[C@H](C2)c2nc(-c3ccc(C(=O)Nc4cc(C(F)(F)F)ccn4)cc3)c3c(N)ncc(n23)/C=C/CCN1]

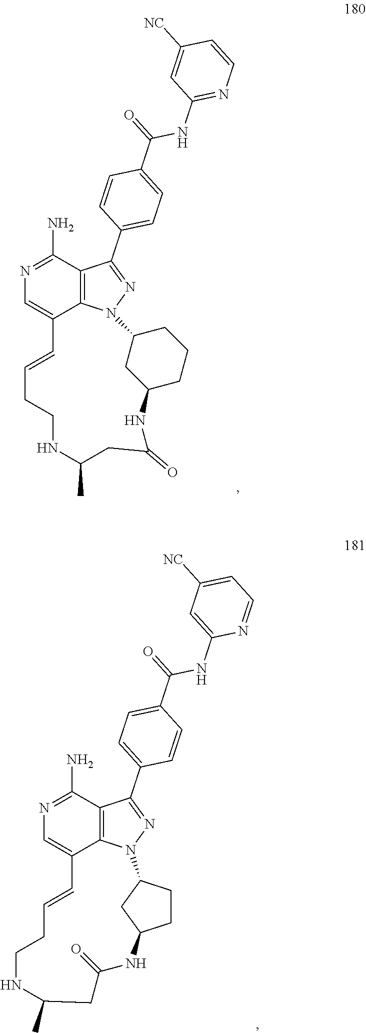 [SMILES: C[C@@H]1CC(=O)N[C@@H]2CCC[C@H](C2)n2nc(-c3ccc(C(=O)Nc4cc(C#N)ccn4)cc3)c3c(N)ncc(c32)/C=C/CCN1 ; C[C@@H]1CC(=O)N[C@@H]2CC[C@H](C2)n2nc(-c3ccc(C(=O)Nc4cc(C#N)ccn4)cc3)c3c(N)ncc(c32)/C=C/CCN1]

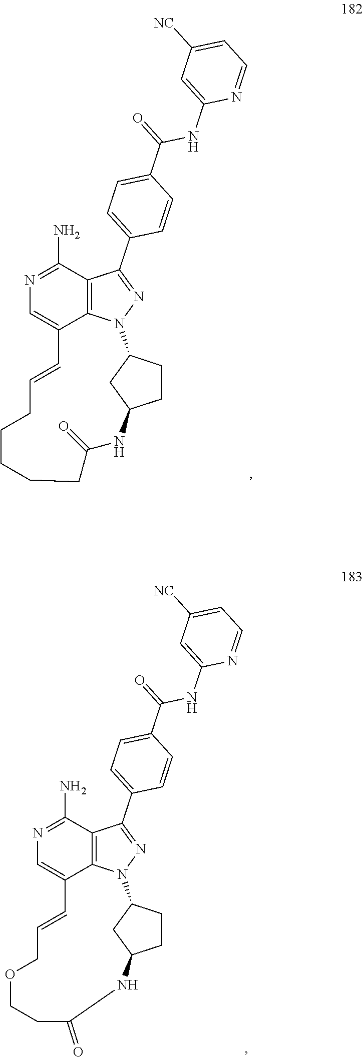 [SMILES: N#Cc1ccnc(NC(=O)c2ccc(-c3nn4c5c(cnc(N)c35)/C=C/CCCCCC(=O)N[C@@H]3CC[C@@H]4C3)cc2)c1 ; N#Cc1ccnc(NC(=O)c2ccc(-c3nn4c5c(cnc(N)c35)/C=C/COCCC(=O)N[C@@H]3CC[C@@H]4C3)cc2)c1]

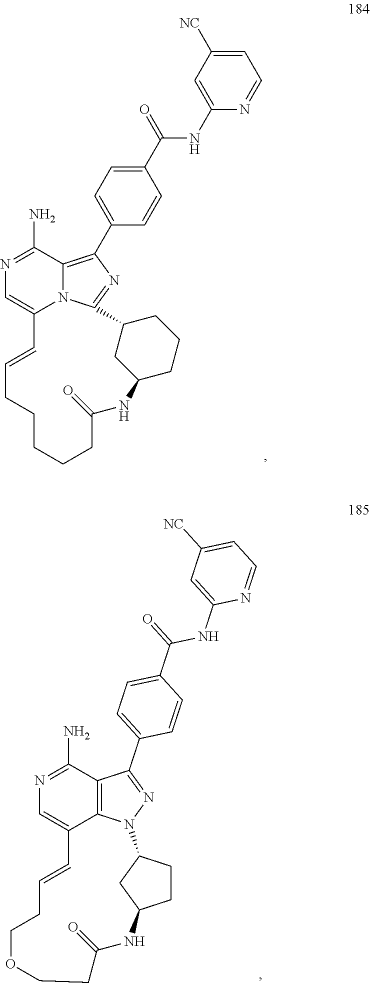 [SMILES: N#Cc1ccnc(NC(=O)c2ccc(-c3nc4n5c(cnc(N)c35)/C=C/CCCCCC(=O)N[C@@H]3CCC[C@@H]4C3)cc2)c1 ; N#Cc1ccnc(NC(=O)c2ccc(-c3nn4c5c(cnc(N)c35)/C=C/CCOCCC(=O)N[C@@H]3CC[C@@H]4C3)cc2)c1]

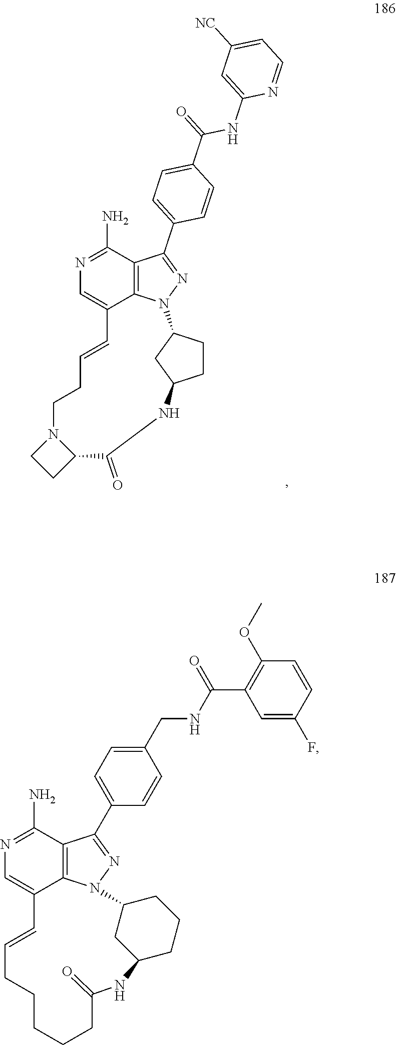 [SMILES: N#Cc1ccnc(NC(=O)c2ccc(-c3nn4c5c(cnc(N)c35)/C=C/CCN3CC[C@H]3C(=O)N[C@@H]3CC[C@@H]4C3)cc2)c1 ; COc1ccc(F)cc1C(=O)NCc1ccc(-c2nn3c4c(cnc(N)c24)/C=C/CCCCCC(=O)N[C@@H]2CCC[C@@H]3C2)cc1]

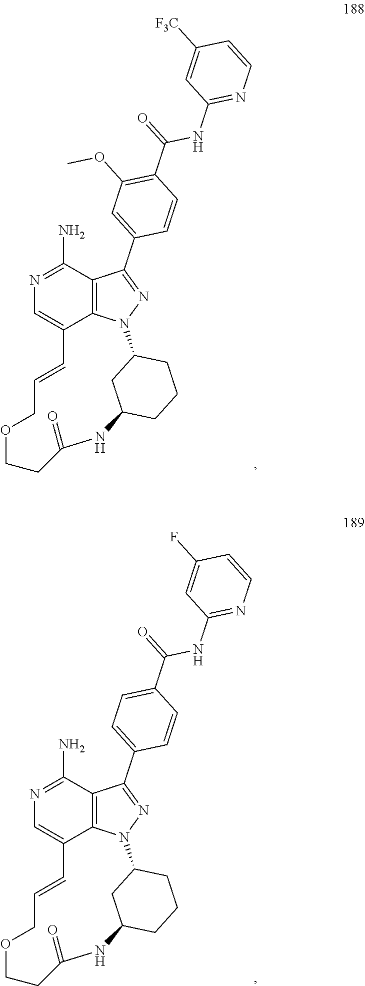 [SMILES: COc1cc(-c2nn3c4c(cnc(N)c24)/C=C/COCCC(=O)N[C@@H]2CCC[C@@H]3C2)ccc1C(=O)Nc1cc(C(F)(F)F)ccn1 ; Nc1ncc2c3c1c(-c1ccc(C(=O)Nc4cc(F)ccn4)cc1)nn3[C@@H]1CCC[C@H](C1)NC(=O)CCOC/C=C/2]

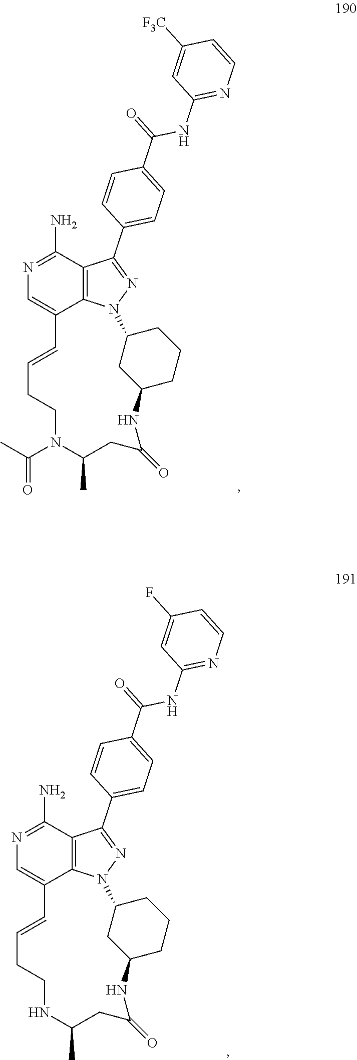 [SMILES: CC(=O)N1CC/C=C/c2cnc(N)c3c(-c4ccc(C(=O)Nc5cc(C(F)(F)F)ccn5)cc4)nn(c23)[C@@H]2CCC[C@H](C2)NC(=O)C[C@H]1C ; C[C@@H]1CC(=O)N[C@@H]2CCC[C@H](C2)n2nc(-c3ccc(C(=O)Nc4cc(F)ccn4)cc3)c3c(N)ncc(c32)/C=C/CCN1]

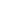

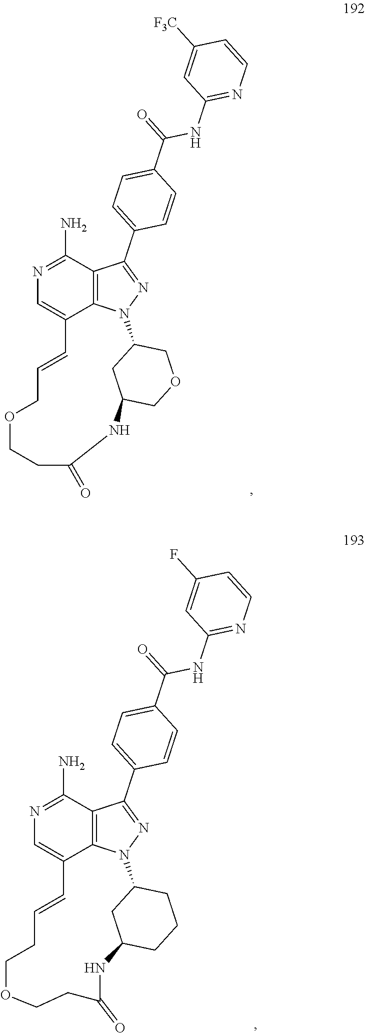 [SMILES: Nc1ncc2c3c1c(-c1ccc(C(=O)Nc4cc(C(F)(F)F)ccn4)cc1)nn3[C@@H]1COC[C@H](C1)NC(=O)CCOC/C=C/2 ; Nc1ncc2c3c1c(-c1ccc(C(=O)Nc4cc(F)ccn4)cc1)nn3[C@@H]1CCC[C@H](C1)NC(=O)CCOCC/C=C/2]

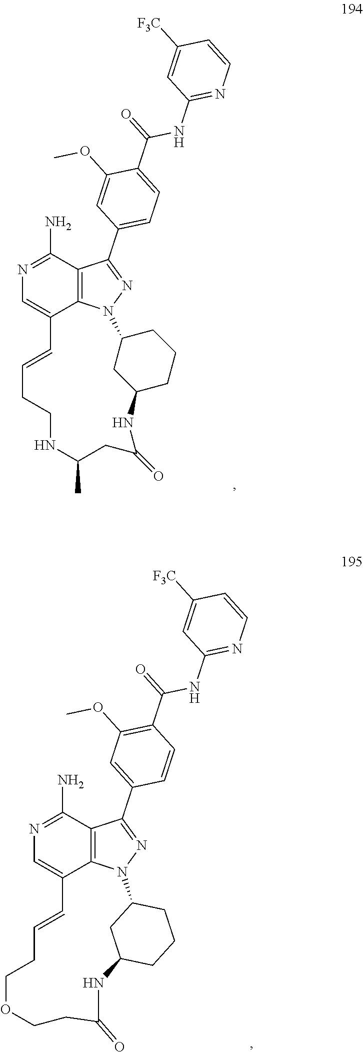 [SMILES: COc1cc(-c2nn3c4c(cnc(N)c24)/C=C/CCN[C@H](C)CC(=O)N[C@@H]2CCC[C@@H]3C2)ccc1C(=O)Nc1cc(C(F)(F)F)ccn1 ; COc1cc(-c2nn3c4c(cnc(N)c24)/C=C/CCOCCC(=O)N[C@@H]2CCC[C@@H]3C2)ccc1C(=O)Nc1cc(C(F)(F)F)ccn1]

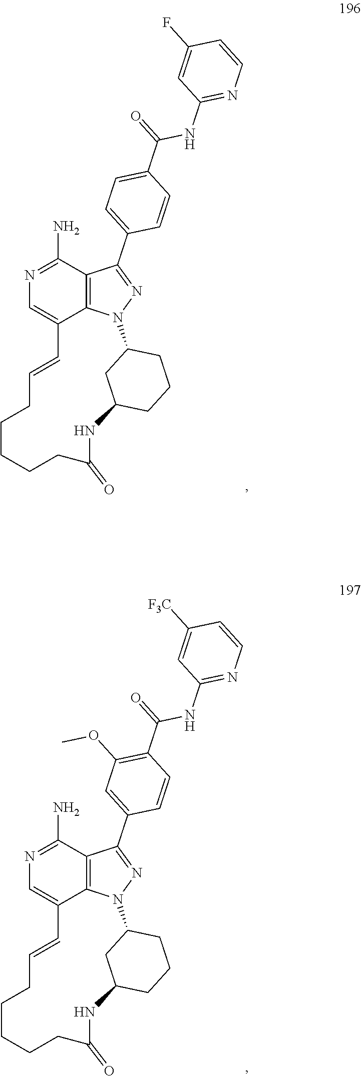 [SMILES: Nc1ncc2c3c1c(-c1ccc(C(=O)Nc4cc(F)ccn4)cc1)nn3[C@@H]1CCC[C@H](C1)NC(=O)CCCCC/C=C/2 ; COc1cc(-c2nn3c4c(cnc(N)c24)/C=C/CCCCCC(=O)N[C@@H]2CCC[C@@H]3C2)ccc1C(=O)Nc1cc(C(F)(F)F)ccn1]

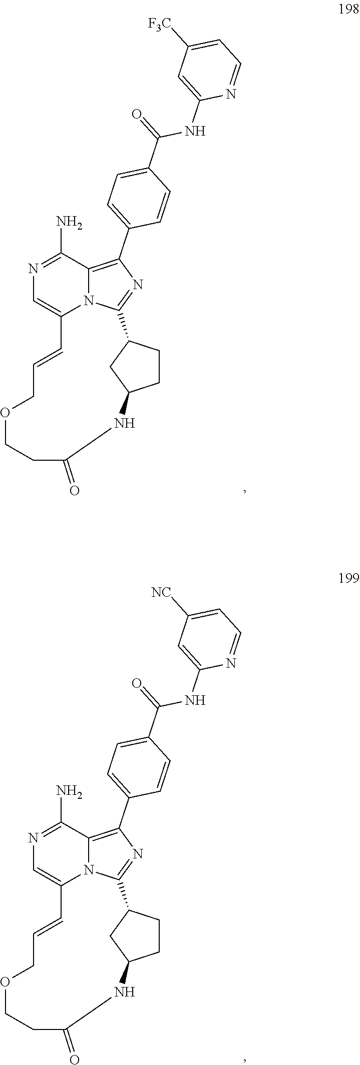 [SMILES: Nc1ncc2n3c(nc(-c4ccc(C(=O)Nc5cc(C(F)(F)F)ccn5)cc4)c13)[C@@H]1CC[C@H](C1)NC(=O)CCOC/C=C/2 ; N#Cc1ccnc(NC(=O)c2ccc(-c3nc4n5c(cnc(N)c35)/C=C/COCCC(=O)N[C@@H]3CC[C@@H]4C3)cc2)c1]

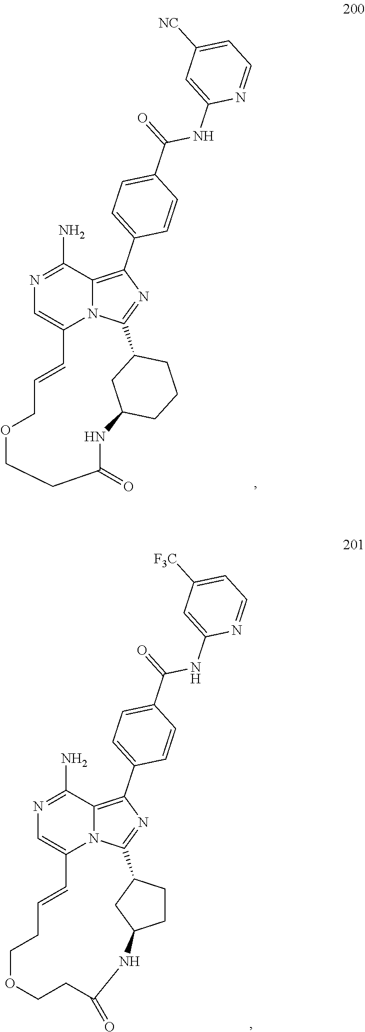 [SMILES: N#Cc1ccnc(NC(=O)c2ccc(-c3nc4n5c(cnc(N)c35)/C=C/COCCC(=O)N[C@@H]3CCC[C@@H]4C3)cc2)c1 ; Nc1ncc2n3c(nc(-c4ccc(C(=O)Nc5cc(C(F)(F)F)ccn5)cc4)c13)[C@@H]1CC[C@H](C1)NC(=O)CCOCC/C=C/2]

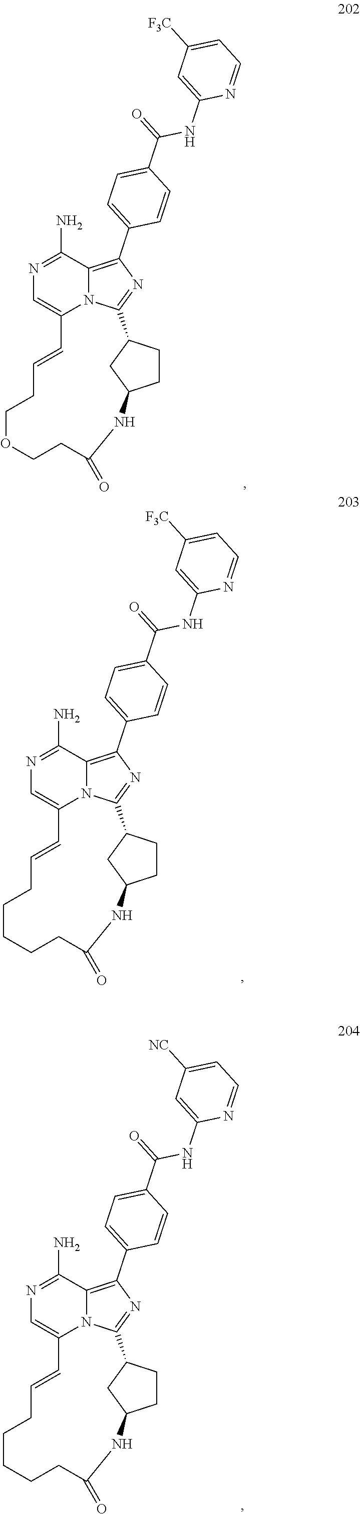 [SMILES: Nc1ncc2n3c(nc(-c4ccc(C(=O)Nc5cc(C(F)(F)F)ccn5)cc4)c13)[C@@H]1CC[C@H](C1)NC(=O)CCOCC/C=C/2 ; Nc1ncc2n3c(nc(-c4ccc(C(=O)Nc5cc(C(F)(F)F)ccn5)cc4)c13)[C@@H]1CC[C@H](C1)NC(=O)CCCCC/C=C/2 ; N#Cc1ccnc(NC(=O)c2ccc(-c3nc4n5c(cnc(N)c35)/C=C/CCCCCC(=O)N[C@@H]3CC[C@@H]4C3)cc2)c1]

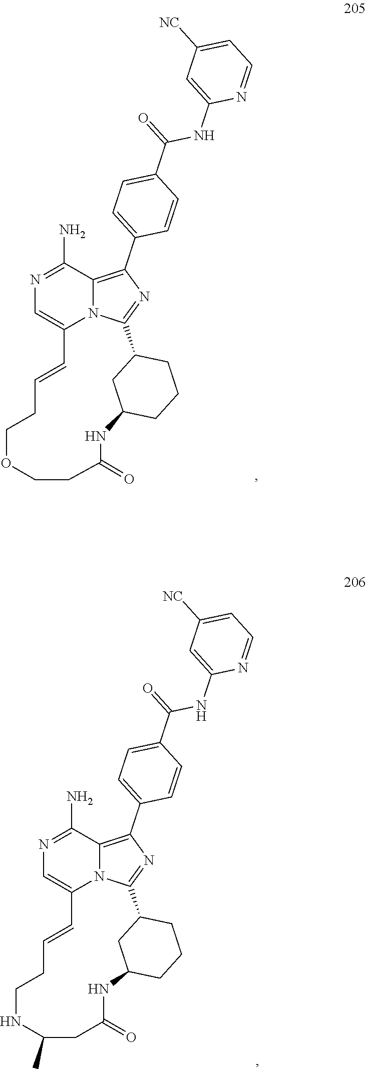 [SMILES: N#Cc1ccnc(NC(=O)c2ccc(-c3nc4n5c(cnc(N)c35)/C=C/CCOCCC(=O)N[C@@H]3CCC[C@@H]4C3)cc2)c1 ; C[C@@H]1CC(=O)N[C@@H]2CCC[C@H](C2)c2nc(-c3ccc(C(=O)Nc4cc(C#N)ccn4)cc3)c3c(N)ncc(n23)/C=C/CCN1]

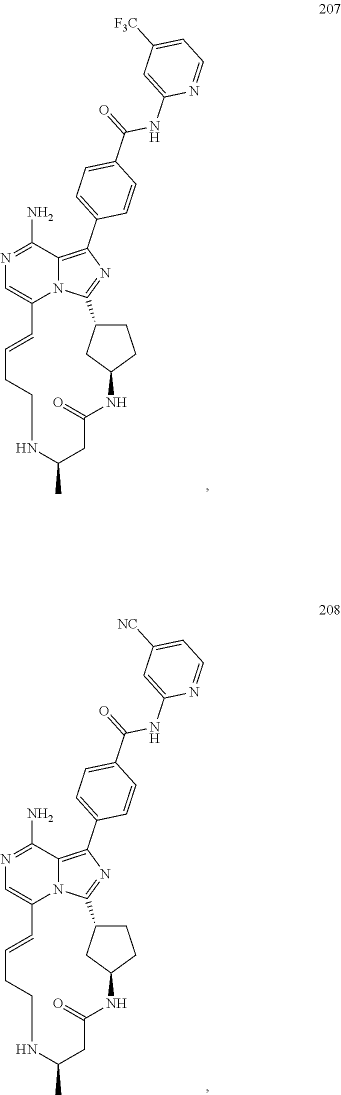 [SMILES: C[C@@H]1CC(=O)N[C@@H]2CC[C@H](C2)c2nc(-c3ccc(C(=O)Nc4cc(C(F)(F)F)ccn4)cc3)c3c(N)ncc(n23)/C=C/CCN1 ; C[C@@H]1CC(=O)N[C@@H]2CC[C@H](C2)c2nc(-c3ccc(C(=O)Nc4cc(C#N)ccn4)cc3)c3c(N)ncc(n23)/C=C/CCN1]

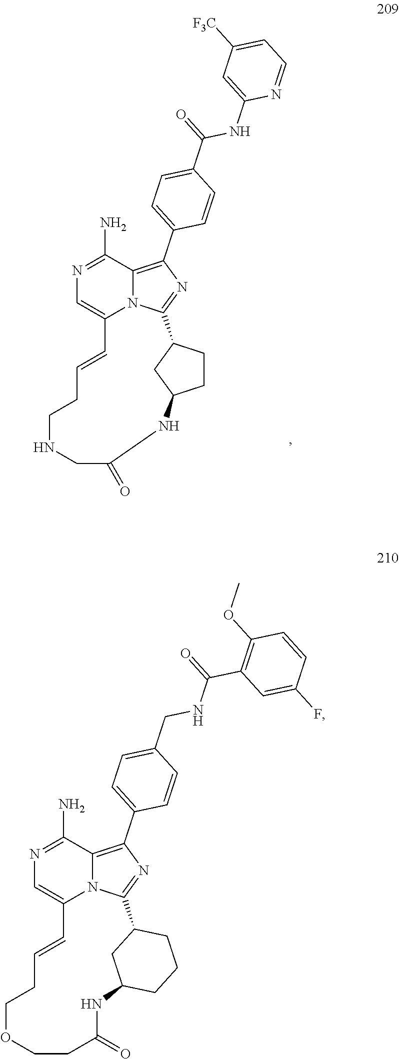 [SMILES: Nc1ncc2n3c(nc(-c4ccc(C(=O)Nc5cc(C(F)(F)F)ccn5)cc4)c13)[C@@H]1CC[C@H](C1)NC(=O)CNCC/C=C/2 ; COc1ccc(F)cc1C(=O)NCc1ccc(-c2nc3n4c(cnc(N)c24)/C=C/CCOCCC(=O)N[C@@H]2CCC[C@@H]3C2)cc1]

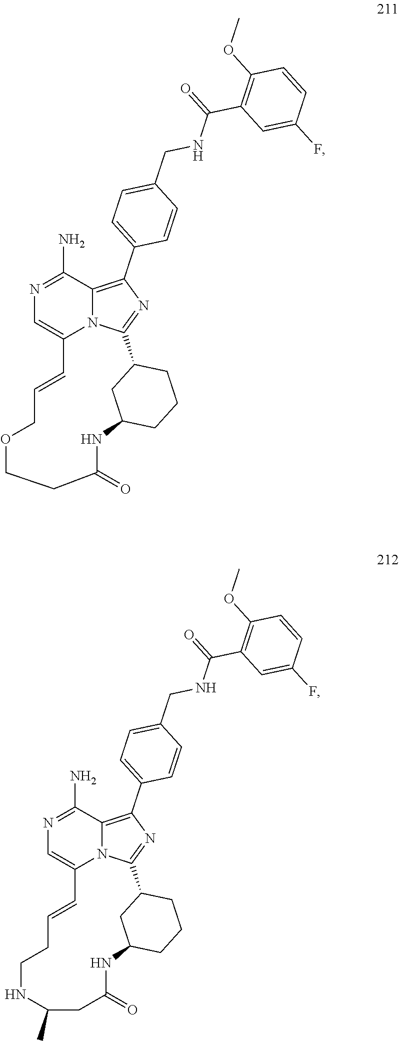 [SMILES: COc1ccc(F)cc1C(=O)NCc1ccc(-c2nc3n4c(cnc(N)c24)/C=C/COCCC(=O)N[C@@H]2CCC[C@@H]3C2)cc1 ; COc1ccc(F)cc1C(=O)NCc1ccc(-c2nc3n4c(cnc(N)c24)/C=C/CCN[C@H](C)CC(=O)N[C@@H]2CCC[C@@H]3C2)cc1]

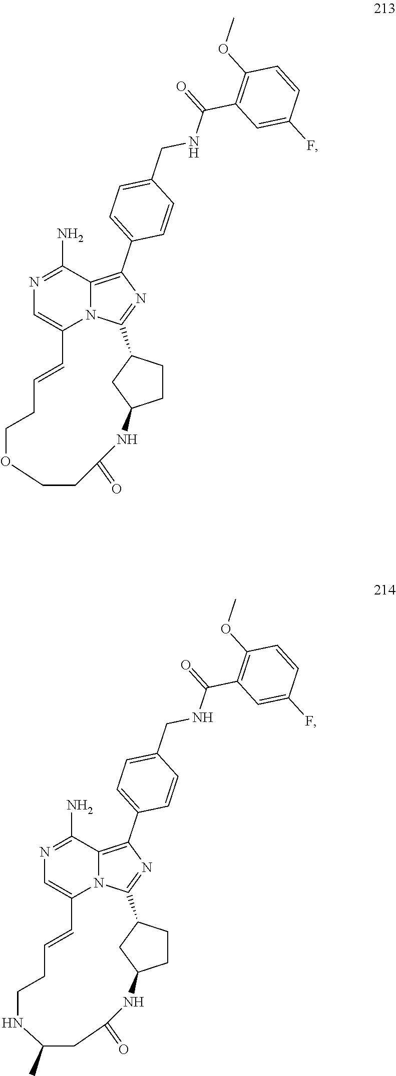 [SMILES: COc1ccc(F)cc1C(=O)NCc1ccc(-c2nc3n4c(cnc(N)c24)/C=C/CCOCCC(=O)N[C@@H]2CC[C@@H]3C2)cc1 ; COc1ccc(F)cc1C(=O)NCc1ccc(-c2nc3n4c(cnc(N)c24)/C=C/CCN[C@H](C)CC(=O)N[C@@H]2CC[C@@H]3C2)cc1]

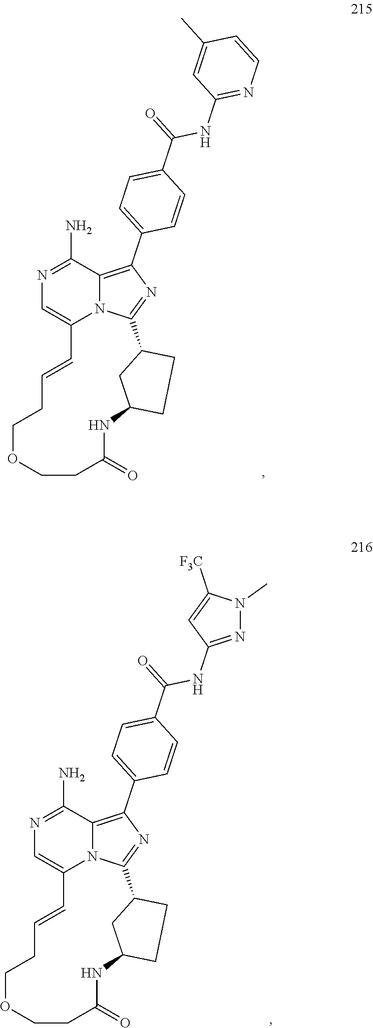 [SMILES: Cc1ccnc(NC(=O)c2ccc(-c3nc4n5c(cnc(N)c35)/C=C/CCOCCC(=O)N[C@@H]3CC[C@@H]4C3)cc2)c1 ; Cn1nc(NC(=O)c2ccc(-c3nc4n5c(cnc(N)c35)/C=C/CCOCCC(=O)N[C@@H]3CC[C@@H]4C3)cc2)cc1C(F)(F)F]

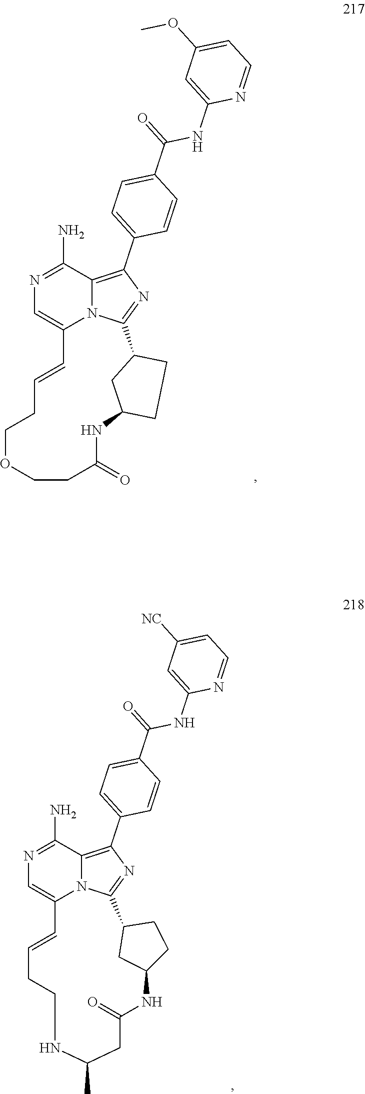 [SMILES: COc1ccnc(NC(=O)c2ccc(-c3nc4n5c(cnc(N)c35)/C=C/CCOCCC(=O)N[C@@H]3CC[C@@H]4C3)cc2)c1 ; C[C@@H]1CC(=O)N[C@@H]2CC[C@H](C2)c2nc(-c3ccc(C(=O)Nc4cc(C#N)ccn4)cc3)c3c(N)ncc(n23)/C=C/CCN1]

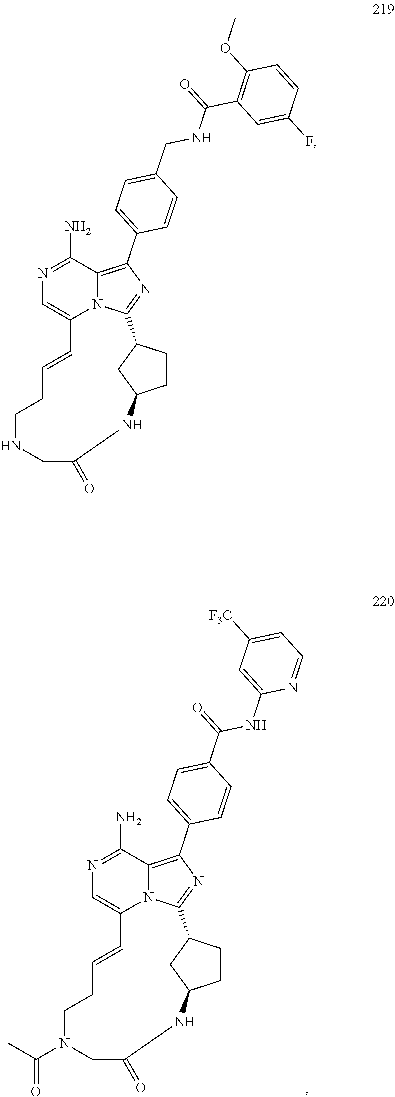 [SMILES: COc1ccc(F)cc1C(=O)NCc1ccc(-c2nc3n4c(cnc(N)c24)/C=C/CCNCC(=O)N[C@@H]2CC[C@@H]3C2)cc1 ; CC(=O)N1CC/C=C/c2cnc(N)c3c(-c4ccc(C(=O)Nc5cc(C(F)(F)F)ccn5)cc4)nc(n23)[C@@H]2CC[C@H](C2)NC(=O)C1]

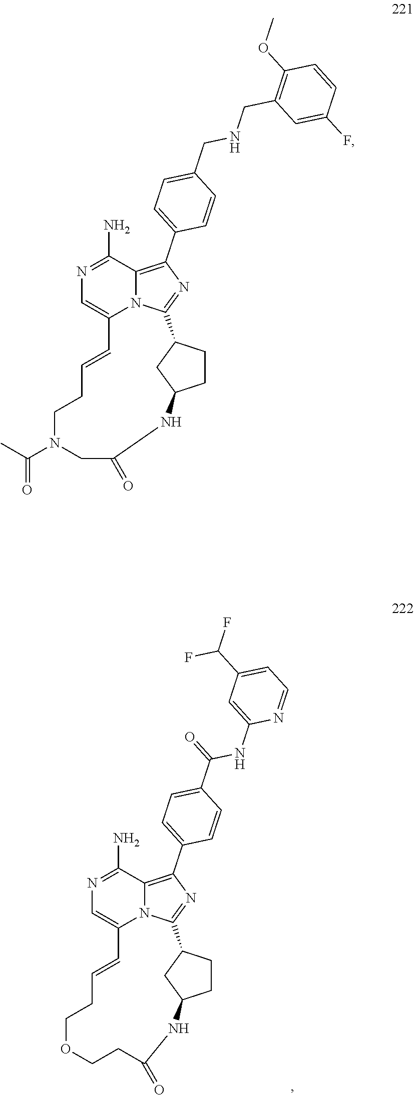 [SMILES: COc1ccc(F)cc1CNCc1ccc(-c2nc3n4c(cnc(N)c24)/C=C/CCN(C(C)=O)CC(=O)N[C@@H]2CC[C@@H]3C2)cc1 ; Nc1ncc2n3c(nc(-c4ccc(C(=O)Nc5cc(C(F)F)ccn5)cc4)c13)[C@@H]1CC[C@H](C1)NC(=O)CCOCC/C=C/2]

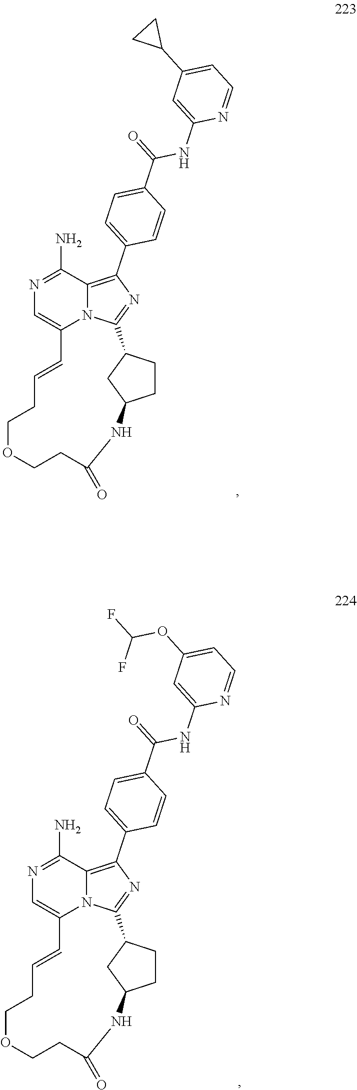 [SMILES: Nc1ncc2n3c(nc(-c4ccc(C(=O)Nc5cc(C6CC6)ccn5)cc4)c13)[C@@H]1CC[C@H](C1)NC(=O)CCOCC/C=C/2 ; Nc1ncc2n3c(nc(-c4ccc(C(=O)Nc5cc(OC(F)F)ccn5)cc4)c13)[C@@H]1CC[C@H](C1)NC(=O)CCOCC/C=C/2]

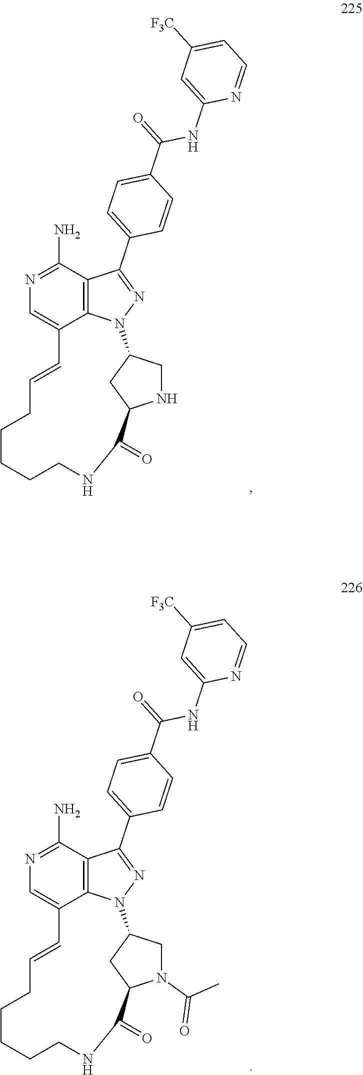 [SMILES: Nc1ncc2c3c1c(-c1ccc(C(=O)Nc4cc(C(F)(F)F)ccn4)cc1)nn3[C@@H]1CN[C@H](C1)C(=O)NCCCCC/C=C/2 ; CC(=O)N1C[C@@H]2C[C@@H]1C(=O)NCCCCC/C=C/c1cnc(N)c3c(-c4ccc(C(=O)Nc5cc(C(F)(F)F)ccn5)cc4)nn2c13]

Further Note that:

Several of the compounds of sub-formulas 1-226 are a cis isomer or trans isomer as indicated in the specific sub-formulas. Additionally, any of said compounds of sub-formulas 1-226 having a cis or trans isomer may also be provided in the alternative (trans or cis) isomer form or may be provided as a mixture of cis and trans isomers.

Compound of sub-formula 11 is an isomer 1 and compound of sub-formula 12 is an isomer 2 of the same structure. Compound of sub-formula 13 is an isomer 1 and compound of sub-formula 14 is an isomer 2 of the same structure. Compound of sub-formula 57 is an isomer 1 and compound of sub-formula 58 is an isomer 2 of the same structure. Compound of sub-formula 143 is an isomer 1 and compound of sub-formula 144 is an isomer 2 of the same structure.

Compound of sub-formula 145 is an isomer 1 and compound of sub-formula 146 is an isomer 2 of the same structure. Compound of sub-formula 156 is an isomer 1 and compound of sub-formula 157 is an isomer 2 of the same structure. Compound of sub-formula 158 is an isomer 1 and compound of sub-formula 159 is an isomer 2 of the same structure. Compound of sub-formula 161 is an isomer 1 and compound of sub-formula 162 is an isomer 2 of the same structure

Compounds of sub-formulas 1-226 may exist as salts with pharmaceutically acceptable acids. The present invention includes such salts. Examples of such salts include hydrochlorides, hydrobromides, sulfates, phosphates, methanesulfonates, nitrates, maleates, acetates, citrates, fumarates, tartrates [e.g. (+)-tartrates, (−)-tartrates or mixtures thereof including racemic mixtures], succinates, benzoates and salts with amino acids such as glutamic acid. These salts may be prepared by methods known to those skilled in the art.

Certain compounds of sub-formulas 1-226 which have acidic substituents may exist as salts with pharmaceutically acceptable bases. The present invention includes such salts. Example of such salts include sodium salts, potassium salts, lysine salts and arginine salts. These salts may be prepared by methods known to those skilled in the art.

Certain compounds of sub-formulas 1-226 and their salts may exist in more than one crystal form and the present invention includes each crystal form and mixtures thereof.

Certain compounds of sub-formulas 1-226 and their salts may also exist in the form of solvates, for example hydrates, and the present invention includes each solvate and mixtures thereof.

The compounds of this invention are useful as inhibitors of tyrosine kinases, in particular they are useful as inhibitors of BTK. In particular, compounds of this invention are useful as inhibitors of tyrosine kinases that are important in hyper-proliferative diseases, especially in cancer and in the process of angiogenesis.

In a preferred embodiment, wherein the compound has a sub-formula (as shown above) selected from the group consisting of: 1-57, 59-64, 68-86, 87b-90, 92-94b, 97b-98b, 99b, 100b-103b, 104b, 108-109a, 110, 112, 114-115, 117-133b, 137-139, 141-143, 146-153, 155-170, 172-188, 191-207, 209-219 and 222-225.

In a more preferred embodiment, wherein the compound has a sub-formula (as shown above) selected from the group consisting of: 1, 2, 6, 7, 8, 9, 10, 11, 17, 18, 20, 21, 24, 25, 26, 27, 28, 33, 34, 39, 40, 44, 45, 46, 47, 48, 49, 50, 51, 52, 53, 54, 56, 57, 59, 61, 68, 71, 76, 79, 83, 85b, 87b, 88b, 89b, 98b, 100b, 114, 115, 130, 131, 132, 133b, 139, 141, 146, 147, 150-153, 155-157, 162-165, 168-170, 172, 174-176, 179-189, 192, 193, 195, 198-205, 207, 210, 213, 215-217 and 222-226.

In a most preferred embodiment, the compound is selected from the group consisting of:

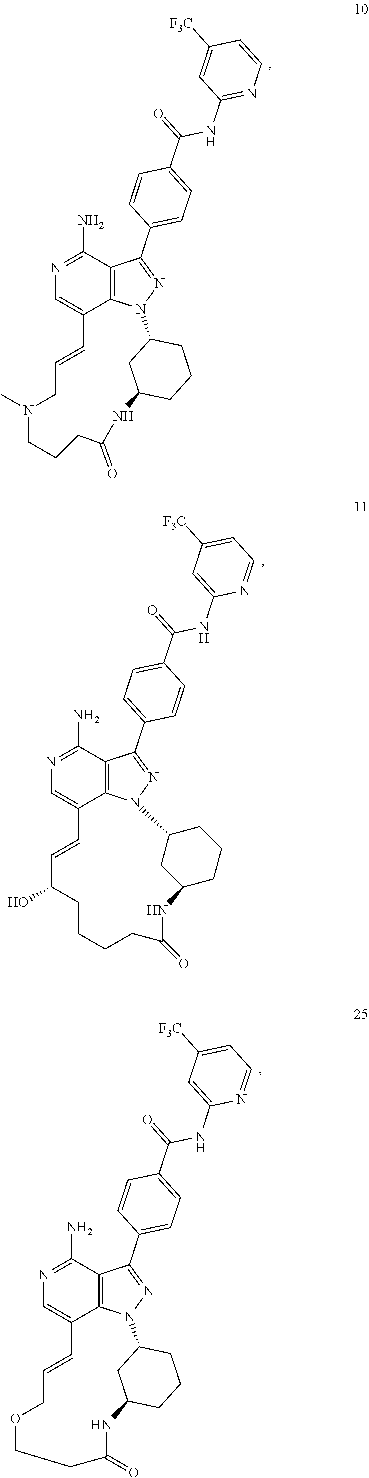 [SMILES: CN1C/C=C/c2cnc(N)c3c(-c4ccc(C(=O)Nc5cc(C(F)(F)F)ccn5)cc4)nn(c23)[C@@H]2CCC[C@H](C2)NC(=O)CCC1 ; Nc1ncc2c3c1c(-c1ccc(C(=O)Nc4cc(C(F)(F)F)ccn4)cc1)nn3[C@@H]1CCC[C@H](C1)NC(=O)CCCC[C@H](O)/C=C/2 ; Nc1ncc2c3c1c(-c1ccc(C(=O)Nc4cc(C(F)(F)F)ccn4)cc1)nn3[C@@H]1CCC[C@H](C1)NC(=O)CCOC/C=C/2]

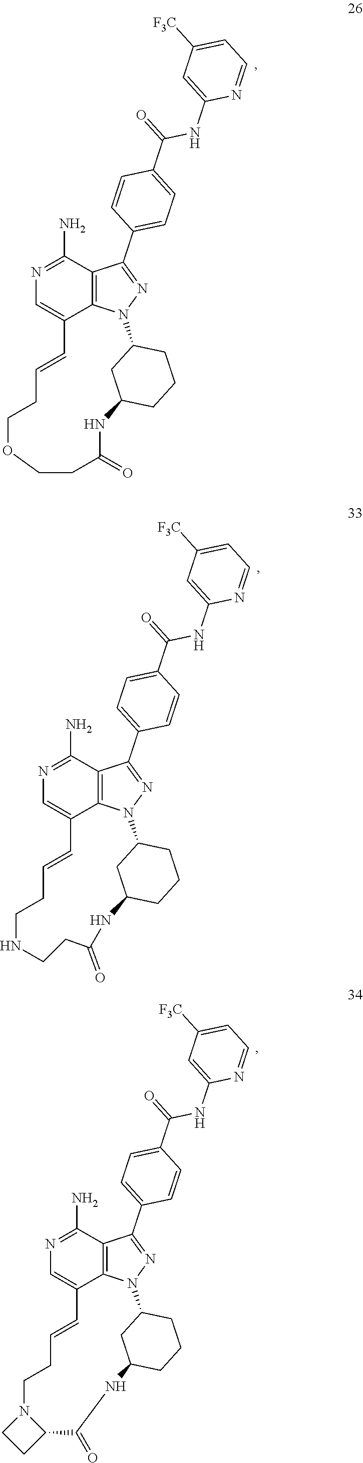 [SMILES: Nc1ncc2c3c1c(-c1ccc(C(=O)Nc4cc(C(F)(F)F)ccn4)cc1)nn3[C@@H]1CCC[C@H](C1)NC(=O)CCOCC/C=C/2 ; Nc1ncc2c3c1c(-c1ccc(C(=O)Nc4cc(C(F)(F)F)ccn4)cc1)nn3[C@@H]1CCC[C@H](C1)NC(=O)CCNCC/C=C/2 ; Nc1ncc2c3c1c(-c1ccc(C(=O)Nc4cc(C(F)(F)F)ccn4)cc1)nn3[C@@H]1CCC[C@H](C1)NC(=O)[C@@H]1CCN1CC/C=C/2]

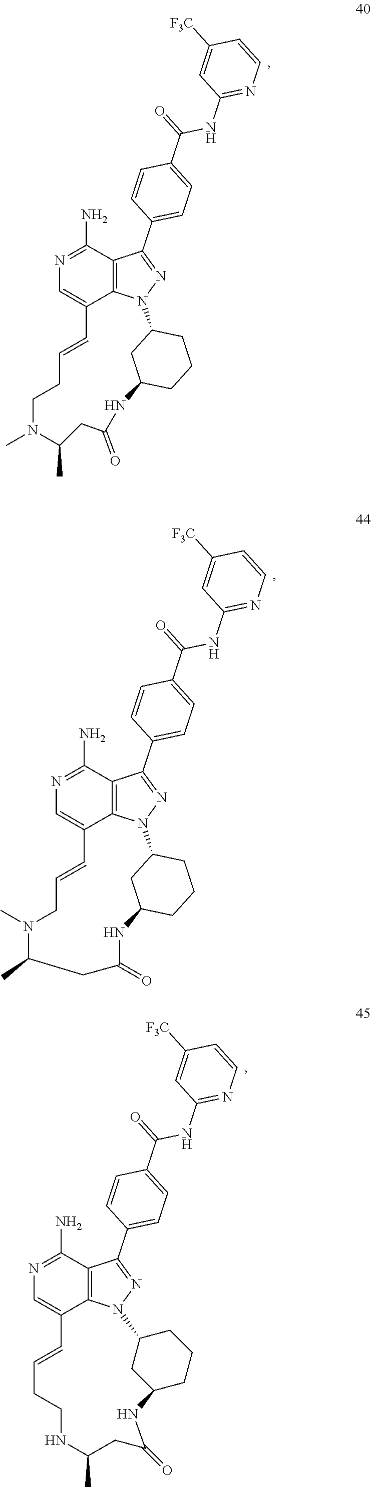 [SMILES: C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCN1C ; C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CN1C ; C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCN1]

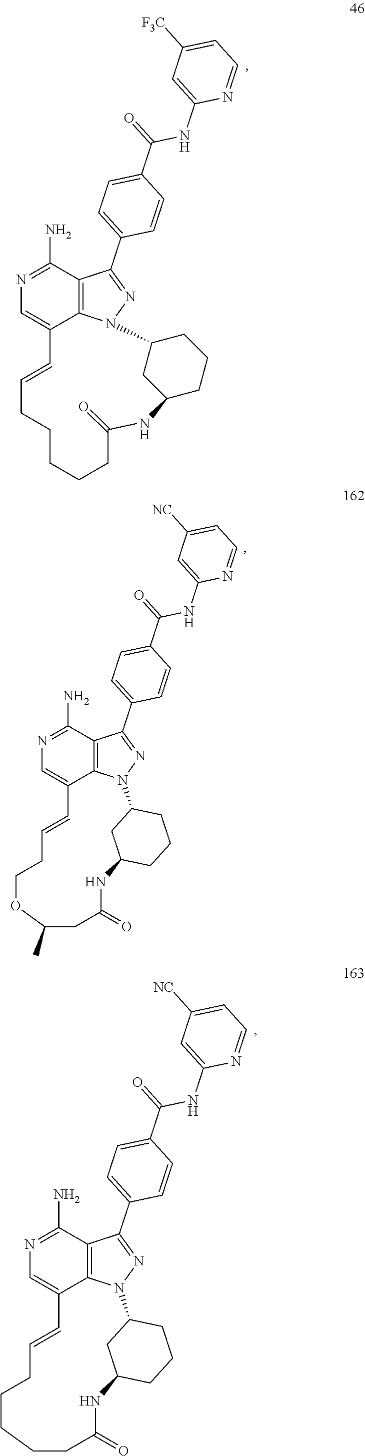 [SMILES: Nc1ncc2c3c1c(-c1ccc(C(=O)Nc4cc(C(F)(F)F)ccn4)cc1)nn3[C@@H]1CCC[C@H](C1)NC(=O)CCCCC/C=C/2 ; C[C@@H]1CC(=O)N[C@@H]2CCC[C@H](C2)n2nc(-c3ccc(C(=O)Nc4cc(C#N)ccn4)cc3)c3c(N)ncc(c32)/C=C/CCO1 ; N#Cc1ccnc(NC(=O)c2ccc(-c3nn4c5c(cnc(N)c35)/C=C/CCCCCC(=O)N[C@@H]3CCC[C@@H]4C3)cc2)c1]

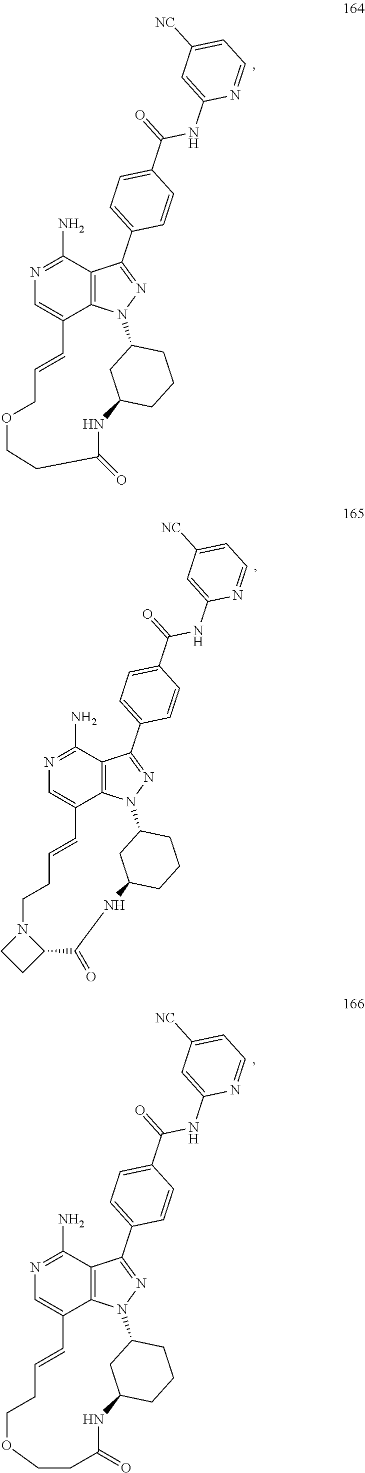 [SMILES: N#Cc1ccnc(NC(=O)c2ccc(-c3nn4c5c(cnc(N)c35)/C=C/COCCC(=O)N[C@@H]3CCC[C@@H]4C3)cc2)c1 ; N#Cc1ccnc(NC(=O)c2ccc(-c3nn4c5c(cnc(N)c35)/C=C/CCN3CC[C@H]3C(=O)N[C@@H]3CCC[C@@H]4C3)cc2)c1 ; N#Cc1ccnc(NC(=O)c2ccc(-c3nn4c5c(cnc(N)c35)/C=C/CCOCCC(=O)N[C@@H]3CCC[C@@H]4C3)cc2)c1]

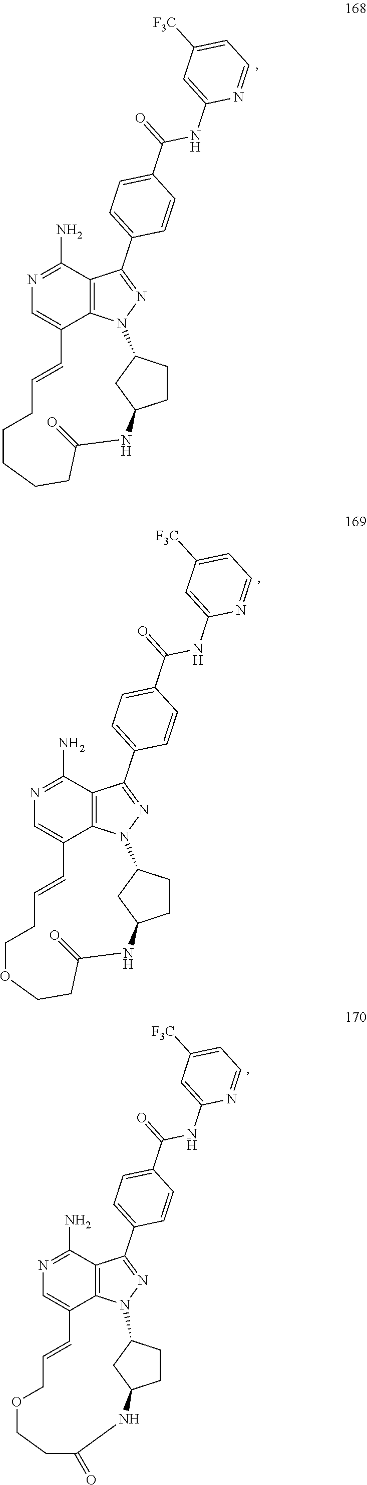 [SMILES: Nc1ncc2c3c1c(-c1ccc(C(=O)Nc4cc(C(F)(F)F)ccn4)cc1)nn3[C@@H]1CC[C@H](C1)NC(=O)CCCCC/C=C/2 ; Nc1ncc2c3c1c(-c1ccc(C(=O)Nc4cc(C(F)(F)F)ccn4)cc1)nn3[C@@H]1CC[C@H](C1)NC(=O)CCOCC/C=C/2 ; Nc1ncc2c3c1c(-c1ccc(C(=O)Nc4cc(C(F)(F)F)ccn4)cc1)nn3[C@@H]1CC[C@H](C1)NC(=O)CCOC/C=C/2]

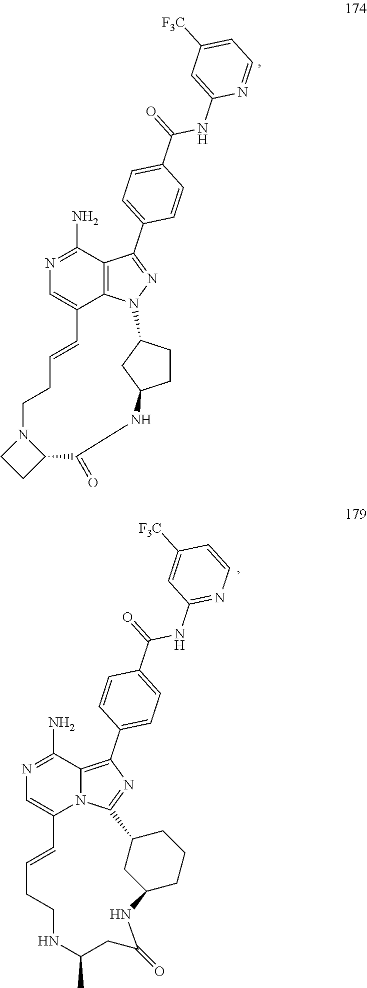 [SMILES: Nc1ncc2c3c1c(-c1ccc(C(=O)Nc4cc(C(F)(F)F)ccn4)cc1)nn3[C@@H]1CC[C@H](C1)NC(=O)[C@@H]1CCN1CC/C=C/2 ; C[C@@H]1CC(=O)N[C@@H]2CCC[C@H](C2)c2nc(-c3ccc(C(=O)Nc4cc(C(F)(F)F)ccn4)cc3)c3c(N)ncc(n23)/C=C/CCN1]

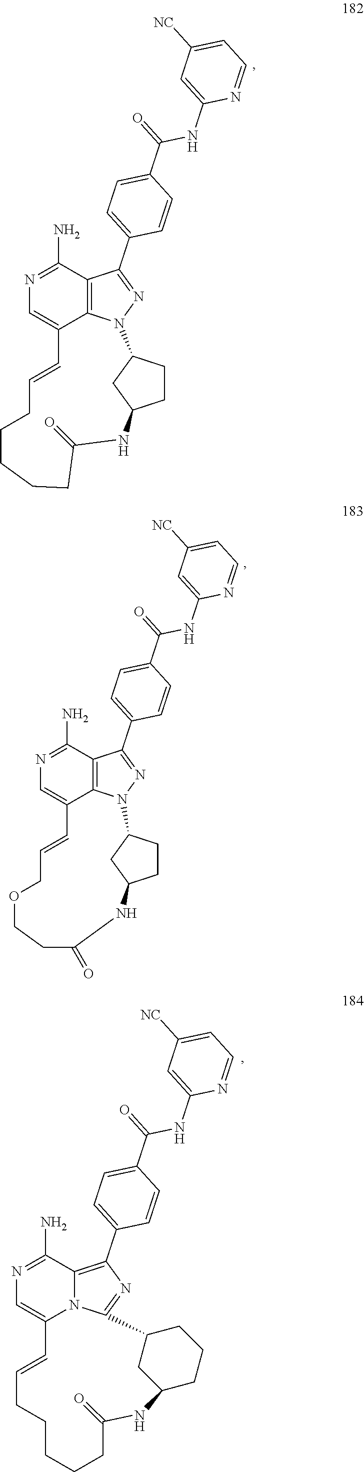 [SMILES: N#Cc1ccnc(NC(=O)c2ccc(-c3nn4c5c(cnc(N)c35)/C=C/CCCCCC(=O)N[C@@H]3CC[C@@H]4C3)cc2)c1 ; N#Cc1ccnc(NC(=O)c2ccc(-c3nn4c5c(cnc(N)c35)/C=C/COCCC(=O)N[C@@H]3CC[C@@H]4C3)cc2)c1 ; N#Cc1ccnc(NC(=O)c2ccc(-c3nc4n5c(cnc(N)c35)/C=C/CCCCCC(=O)N[C@@H]3CCC[C@@H]4C3)cc2)c1]

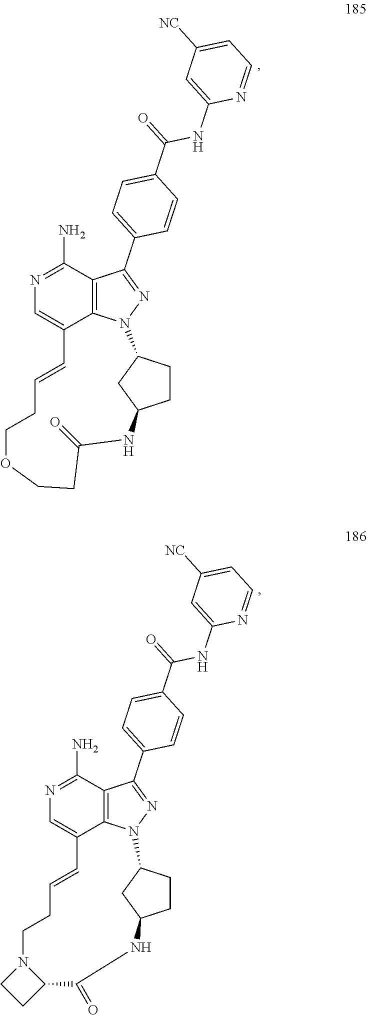 [SMILES: N#Cc1ccnc(NC(=O)c2ccc(-c3nn4c5c(cnc(N)c35)/C=C/CCOCCC(=O)N[C@@H]3CC[C@@H]4C3)cc2)c1 ; N#Cc1ccnc(NC(=O)c2ccc(-c3nn4c5c(cnc(N)c35)/C=C/CCN3CC[C@H]3C(=O)N[C@@H]3CC[C@@H]4C3)cc2)c1]

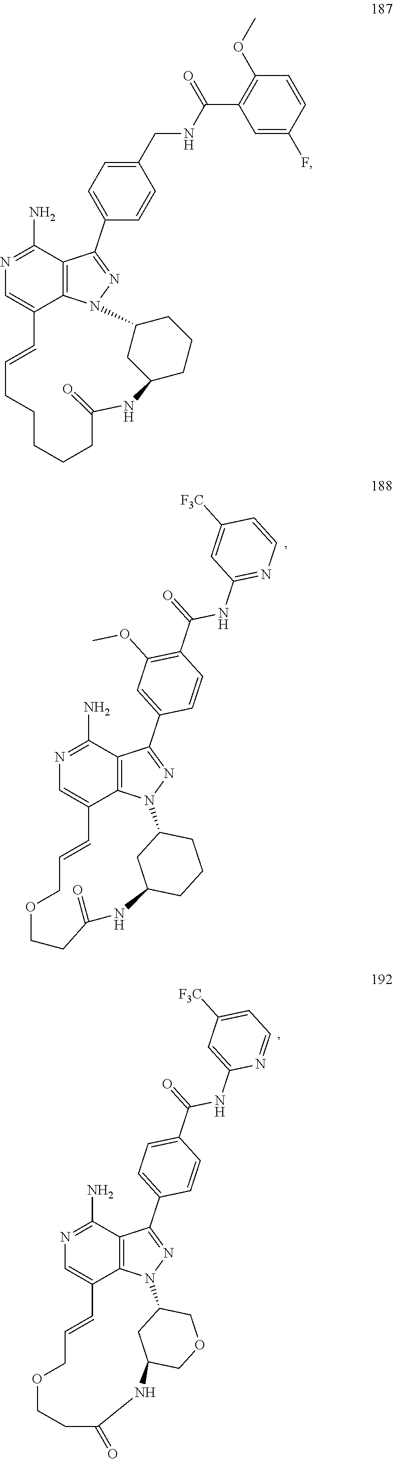 [SMILES: COc1ccc(F)cc1C(=O)NCc1ccc(-c2nn3c4c(cnc(N)c24)/C=C/CCCCCC(=O)N[C@@H]2CCC[C@@H]3C2)cc1 ; COc1cc(-c2nn3c4c(cnc(N)c24)/C=C/COCCC(=O)N[C@@H]2CCC[C@@H]3C2)ccc1C(=O)Nc1cc(C(F)(F)F)ccn1 ; Nc1ncc2c3c1c(-c1ccc(C(=O)Nc4cc(C(F)(F)F)ccn4)cc1)nn3[C@@H]1COC[C@H](C1)NC(=O)CCOC/C=C/2]

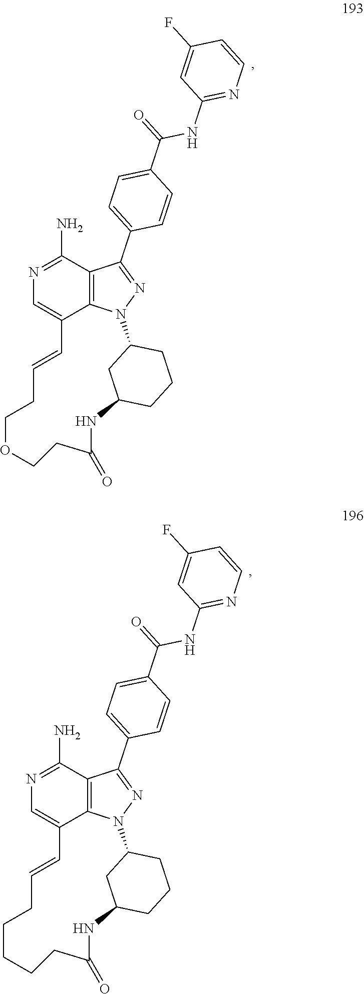 [SMILES: Nc1ncc2c3c1c(-c1ccc(C(=O)Nc4cc(F)ccn4)cc1)nn3[C@@H]1CCC[C@H](C1)NC(=O)CCOCC/C=C/2 ; Nc1ncc2c3c1c(-c1ccc(C(=O)Nc4cc(F)ccn4)cc1)nn3[C@@H]1CCC[C@H](C1)NC(=O)CCCCC/C=C/2]

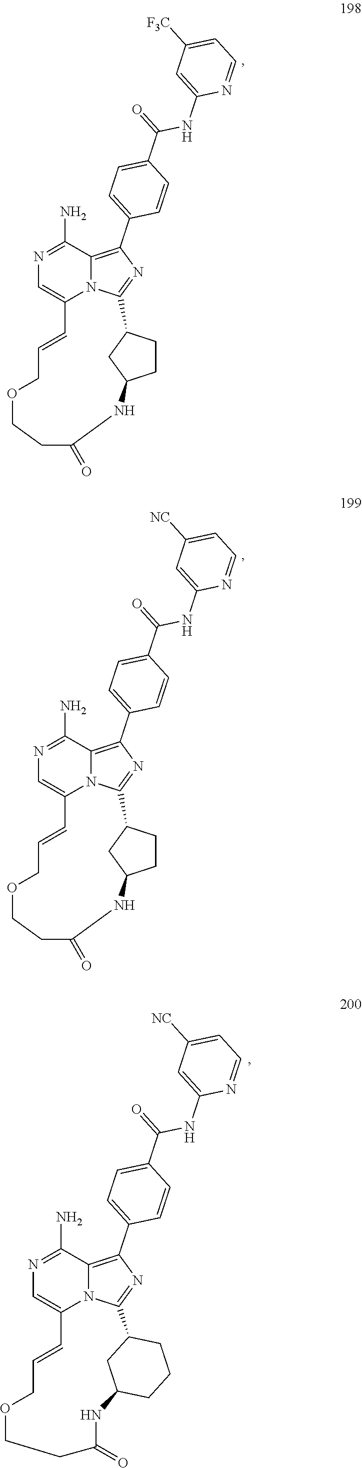 [SMILES: Nc1ncc2n3c(nc(-c4ccc(C(=O)Nc5cc(C(F)(F)F)ccn5)cc4)c13)[C@@H]1CC[C@H](C1)NC(=O)CCOC/C=C/2 ; N#Cc1ccnc(NC(=O)c2ccc(-c3nc4n5c(cnc(N)c35)/C=C/COCCC(=O)N[C@@H]3CC[C@@H]4C3)cc2)c1 ; N#Cc1ccnc(NC(=O)c2ccc(-c3nc4n5c(cnc(N)c35)/C=C/COCCC(=O)N[C@@H]3CCC[C@@H]4C3)cc2)c1]

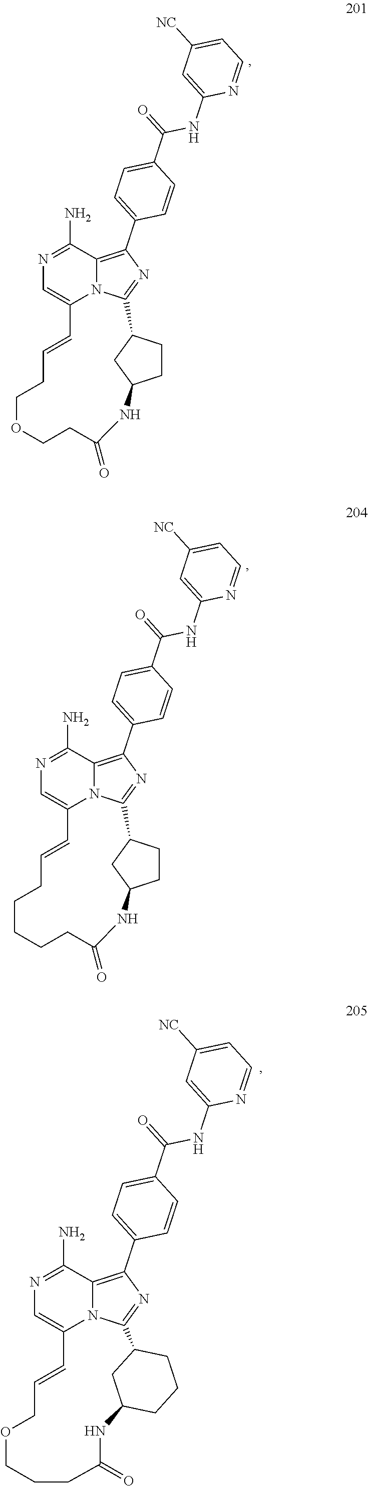 [SMILES: N#Cc1ccnc(NC(=O)c2ccc(-c3nc4n5c(cnc(N)c35)/C=C/CCOCCC(=O)N[C@@H]3CC[C@@H]4C3)cc2)c1 ; N#Cc1ccnc(NC(=O)c2ccc(-c3nc4n5c(cnc(N)c35)/C=C/CCCCCC(=O)N[C@@H]3CC[C@@H]4C3)cc2)c1 ; N#Cc1ccnc(NC(=O)c2ccc(-c3nc4n5c(cnc(N)c35)/C=C/COCCCC(=O)N[C@@H]3CCC[C@@H]4C3)cc2)c1]

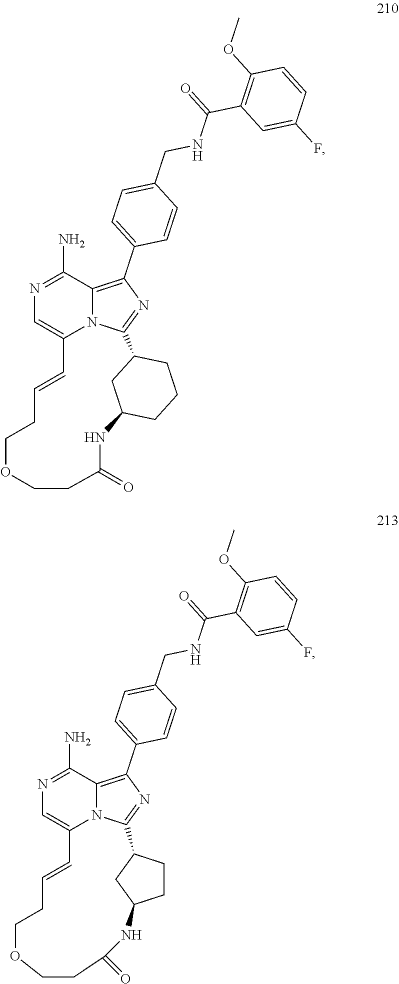 [SMILES: COc1ccc(F)cc1C(=O)NCc1ccc(-c2nc3n4c(cnc(N)c24)/C=C/CCOCCC(=O)N[C@@H]2CCC[C@@H]3C2)cc1 ; COc1ccc(F)cc1C(=O)NCc1ccc(-c2nc3n4c(cnc(N)c24)/C=C/CCOCCC(=O)N[C@@H]2CC[C@@H]3C2)cc1]

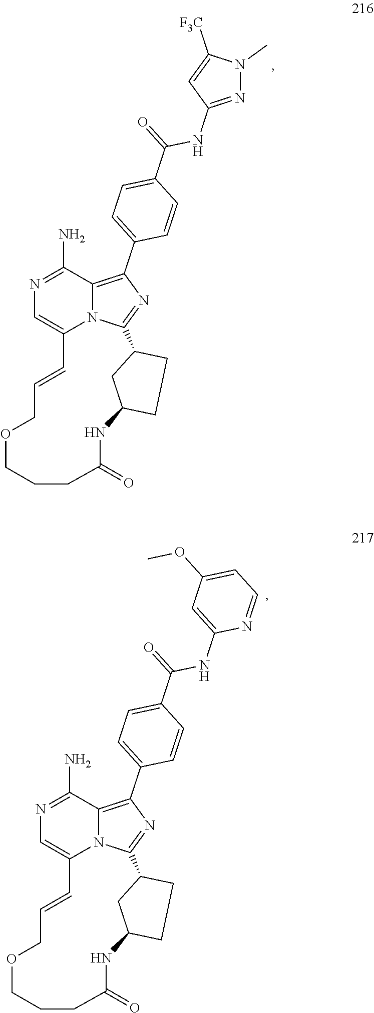 [SMILES: Cn1nc(NC(=O)c2ccc(-c3nc4n5c(cnc(N)c35)/C=C/COCCCC(=O)N[C@@H]3CC[C@@H]4C3)cc2)cc1C(F)(F)F ; COc1ccnc(NC(=O)c2ccc(-c3nc4n5c(cnc(N)c35)/C=C/COCCCC(=O)N[C@@H]3CC[C@@H]4C3)cc2)c1]

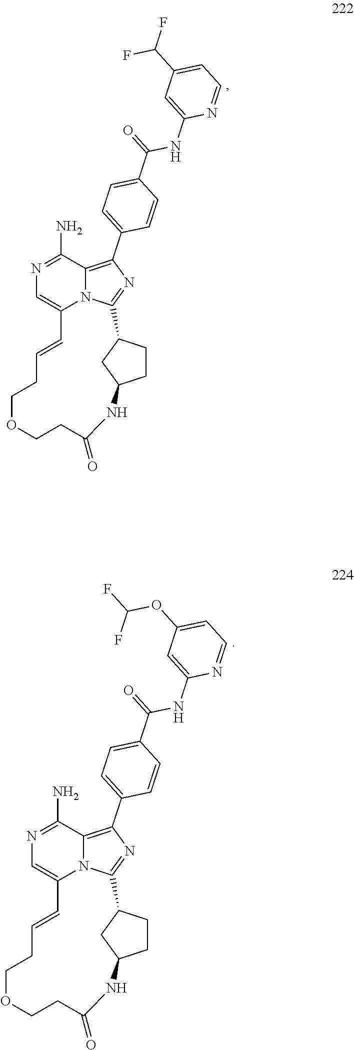 [SMILES: Nc1ncc2n3c(nc(-c4ccc(C(=O)Nc5cc(C(F)F)ccn5)cc4)c13)[C@@H]1CC[C@H](C1)NC(=O)CCOCC/C=C/2 ; Nc1ncc2n3c(nc(-c4ccc(C(=O)Nc5cc(OC(F)F)ccn5)cc4)c13)[C@@H]1CC[C@H](C1)NC(=O)CCOCC/C=C/2]

### Pharmaceutical Composition

Pharmaceutical compositions in accordance with the present invention comprise, as the active ingredient (‘API’), compound of Formula (I-a) to (I-h) or a pharmaceutically acceptable salt, hydrate or solvate thereof.

As used herein, “a pharmaceutically acceptable salt” includes any salt that retains the activity of the active agent(s) and is acceptable for pharmaceutical use. A pharmaceutically acceptable salt also refers to any salt which may form in vivo as a result of administration of an acid, another salt, or a prodrug which is converted into an acid or salt. Preferably, the pharmaceutically acceptable salt is the HCl-salt of the compound of the invention. The pharmaceutically acceptable salt of the disclosed compounds may be prepared by methods of pharmacy well known to those skilled in the art.

Furthermore, the compositions can comprise compounds according to the invention in the form of a solvate, comprising a pharmaceutically acceptable solvent, such as water (‘hydrate’), ethanol, and the like. In general, the solvated forms are considered equivalent to the unsolvated forms for the purposes of the present invention.

As used herein, the term “pharmaceutical composition” refers to a composition comprising a compound according to the invention or a salt or solvate thereof and, as the case may be, one or more additional, non-toxic ingredients, which composition is in a form suitable for administration to a (human) subject, through any route of administration, and which composition is physiologically tolerated upon such administration.

The compositions of the invention may thus comprise one or more additional ingredients. In a preferred embodiment, the composition comprises one or more carriers and/or excipients. As is known by those of average skill in the art, the appropriate choice of excipients is dependent on multiple factors, including the physicochemical properties of the API, the preferred pharmaceutical form, the preferred route of administration, the desired rate of release, etc. The compositions of the invention can be formulated for a variety of routes of administration, oral administration being particularly preferred. It is within the purview of those of average skill in the art to conceive and develop suitable formulations, relying on the common general knowledge as reflected in text books such as Remington's Pharmaceutical Sciences (Meade Publishing Co., Easton, Pa., 20.sup. th Ed., 2000), the entire disclosure of which is herein incorporated by reference, and routine development efforts.

In accordance with the various aspects of the invention, the composition is preferably provided in a unit dosage form. The term ‘unit dosage form’ refers to a physically discrete unit suitable as a unitary dosage for human subjects, each unit containing a predetermined quantity of active material calculated to produce the desired therapeutic effect in association with any suitable pharmaceutical carrier(s) and/or excipient(s). Exemplary, non-limiting unit dosage forms include a tablet (e.g., a chewable tablet), caplet, capsule (e.g., a hard capsule or a soft capsule), lozenge, film, strip, gelcap as well as any metered volume of a solution, suspension, syrup or elixir or the like, which may be contained, for instance in a vial, syringe, applicator device, sachet, spray, micropump etc. In accordance with particularly preferred embodiments of the invention, the unit dosage form, is a unit dosage form that is suitable for oral administration. Most preferably, it is a solid unit dosage form, such as a tablet.

Besides the compound according to the invention as such, pharmaceutically acceptable salts thereof may also be used. Pharmaceutically acceptable salts of compounds of the invention include the acid addition and base salts thereof, such as preferably the calcium, potassium or sodium salts. For a review on suitable salts, reference is made “Handbook of Pharmaceutical Salts: Properties, Selection, and Use” by Stahl and Wermuth (Wiley-VCH, Weinheim, Germany, 2002).

Pharmaceutically acceptable salts of compounds according to the invention may be readily prepared by mixing together solutions of compounds according to the invention and the desired acid or base, as appropriate. The salt may precipitate from solution and be collected by filtration or may be recovered by evaporation of the solvent. The degree of ionisation in the salt may vary from completely ionised to almost non-ionised.

### Medical Use

The compounds and the pharmaceutical compositions of the present invention are useful as inhibitors of tyrosine kinases, in particular of BTK. In particular, compounds of this invention are useful as inhibitors of tyrosine kinases that are important in hyper-proliferative diseases, especially in cancer and in the process of angiogenesis.

The compounds of the present invention are also useful in the treatment of cancer related indications such as solid tumors, sarcomas (especially Ewing's sarcoma and osteosarcoma), retinoblastoma, rhabdomyosarcomas, neuroblastoma, hematopoietic malignancies, including leukaemia and lymphoma, tumor-induced pleural or pericardial effusions, and malignant ascites.

The compounds according to the invention having Formula (I-a) to (I-h) and pharmaceutical compositions thereof can be used to treat or prevent a variety of conditions, diseases or disorders mediated by Bruton's Tyrosine kinase (BTK).

Such conditions, diseases or disorders include: (1) arthritis, including rheumatoid arthritis, juvenile arthritis, psoriatic arthritis and osteoarthritis; (2) asthma and other obstructive airways diseases, including chronic asthma, late asthma, airway hyper-responsiveness, bronchitis, bronchial asthma, allergic asthma, intrinsic asthma, extrinsic asthma, dust asthma, adult respiratory distress syndrome, recurrent airway obstruction, and chronic obstruction pulmonary disease including emphysema; (3) autoimmune diseases or disorders, including those designated as single organ or single cell-type autoimmune disorders, for example Hashimoto's thyroiditis, autoimmune hemolytic anemia, autoimmune atrophic gastritis of pernicious anemia, autoimmune encephalomyelitis, autoimmune orchitis, Goodpasture's disease, autoimmune thrombocytopenia including idiopathic thrombopenic purpura, sympathetic ophthalmia, myasthenia gravis. Graves' disease, primary biliary cirrhosis, chronic aggressive hepatitis, ulcerative colitis and membranous glomerulopathy, those designated as involving systemic autoimmune disorder, for example systemic lupus erythematosis, immune thrombocytopenic purpura, rheumatoid arthritis, Sjogren's syndrome, Reiter's syndrome, polymyositis-dermatomyositis, systemic sclerosis, polyarteritis nodosa, multiple sclerosis and bullous pemphigoid, and additional autoimmune diseases, which can be B-cell (humoral) based or T-cell based, including Cogan's syndrome, ankylosing spondylitis, Wegener's granulomatosis, autoimmune alopecia, Type I or juvenile onset diabetes, and thyroiditis;

(4) cancers or tumors, including alimentary/gastrointestinal tract cancer, colon cancer, liver cancer, skin cancer including mast cell tumor and squamous cell carcinoma, breast and mammary cancer, ovarian cancer, prostate cancer, lymphoma and leukemia (including but not limited to acute myelogenous leukemia, chronic myelogenous leukemia, mantle cell lymphoma, NHL B cell lymphomas (e.g. precursor B-ALL, marginal zone B cell lymphoma, chronic lymphocytic leukemia, diffuse large B cell lymphoma, Burkitt lymphoma, mediastinal large B-cell lymphoma), Hodgkin lymphoma, NK and T cell lymphomas, TEL-Syk and ITK-Syk fusion driven tumors, myelomas including multiple myeloma, myeloproliferative disorders kidney cancer, lung cancer, muscle cancer, bone cancer, bladder cancer, brain cancer, melanoma including oral and metastatic melanoma, Kaposi's sarcoma, proliferative diabetic retinopathy, and angiogenic-associated disorders including solid tumors, and pancreatic cancer.

(5) diabetes, including Type I diabetes and complications from diabetes; (6) eye diseases, disorders or conditions including autoimmune diseases of the eye, keratoconjunctivitis, vernal conjunctivitis, uveitis including uveitis associated with Behcet's disease and lens-induced uveitis, keratitis, herpetic keratitis, conical keratitis, corneal epithelial dystrophy, keratoleukoma, ocular premphigus, Mooren's ulcer, scleritis, Grave's ophthalmopathy, Vogt-Koyanagi-Harada syndrome, keratoconjunctivitis sicca (dry eye), phlyctenule, iridocyclitis, sarcoidosis, endocrine ophthalmopathy, sympathetic ophthalmitis, allergic conjunctivitis, and ocular neovascularization; (7) intestinal inflammations, allergies or conditions including Crohn's disease and/or ulcerative colitis, inflammatory bowel disease, coeliac diseases, proctitis, eosinophilic gastroenteritis, and mastocytosis; (8) neurodegenerative diseases including motor neuron disease, Alzheimer's disease, Parkinson's disease, amyotrophic lateral sclerosis, Huntington's disease, cerebral ischemia, or neurodegenerative disease caused by traumatic injury, strike, glutamate neurotoxicity or hypoxia; ischemic/reperfusion injury in stroke, myocardial is chemica, renal ischemia, heart attacks, cardiac hypertrophy, atherosclerosis and arteriosclerosis, organ hypoxia; (9) platelet aggregation and diseases associated with or caused by platelet activation, such as arteriosclerosis, thrombosis, intimal hyperplasia and restenosis following vascular injury; (10) conditions associated with cardiovascular diseases.

In a preferred embodiment, the compounds according to the invention having Formula (I-a) to (I-h) and pharmaceutical compositions thereof can be used to treat or prevent a Bruton's Tyrosine Kinase (BTK) mediated disorder, wherein the Bruton's Tyrosine Kinase (BTK) mediated disorder is selected from the group consisting of an allergic disease, an autoimmune disease, an inflammatory disease, a thromboembolic disease, a bone-related disease, and cancer.

In a preferred embodiment, the compounds according to the invention having Formula (I-a) to (I-h) and pharmaceutical compositions thereof can be used in the treatment of cancer, lymphoma or leukemia.

In preferred embodiments, the compounds according to the invention having Formula (I-a) to (I-h) and pharmaceutical compositions thereof can be used to treat or prevent a Bruton's Tyrosine Kinase (BTK) mediated disorder, wherein the Bruton's Tyrosine Kinase (BTK) mediated disorder is selected from the group consisting of B-cell malignancy, B-cell lymphoma, diffuse large B-cell lymphoma, chronic lymphocyte leukemia, non-Hodgkin lymphoma for example ABC-DLBCL, mantle cell lymphoma, follicular lymphoma, hairy cell leukemia B-cell non-Hodgkin lymphoma, Waldenstrom's macroglobulinemia, Richter transformation, multiple myeloma, bone cancer, bone metastasis, chronic lymphocytic lymphomas, B-cell prolymphocyte leukemia, lymphoplasmacytic lymphoma, splenic marginal zone lymphoma, plasma cell lymphoma, plasmacytoma, extranodal marginal zone B-cell lymphoma, nodal marginal zone B-cell lymphoma, mediastinal (thymic) large B-cell lymphoma, intravascular large B-cell lymphoma, primary effusion lymphoma, Burkitt lymphoma/leukemia, and lymphomatoid granulomatosis.

In preferred embodiments, the compounds according to the invention having Formula (I-a) to (I-h) and pharmaceutical compositions thereof can be used to treat or prevent a Bruton's Tyrosine Kinase (BTK) mediated disorder, wherein the Bruton's Tyrosine Kinase (BTK) mediated disorder is selected from the group consisting of rheumatoid arthritis, psoriatic arthritis, infectious arthritis, progressive chronic arthritis, deforming arthritis, osteoarthritis, traumatic arthritis, gouty arthritis, Reiter's syndrome, polychondritis, acute synovitis and spondylitis, glomerulonephritis (with or without nephrotic syndrome), autoimmune hematologic disorders, hemolytic anemia, aplasic anemia, idiopathic thrombocytopenia, and neutropenia, autoimmune gastritis, and autoimmune inflammatory bowel diseases, ulcerative colitis, Crohn's disease, host versus graft disease, allograft rejection, chronic thyroiditis, Graves' disease, schleroderma, diabetes (type I and type II), active hepatitis (acute and chronic), pancreatitis, primary billiary cirrhosis, myasthenia gravis, multiple sclerosis, systemic lupus erythematosis, psoriasis, atopic dermatitis, contact dermatitis, eczema, skin sunburns, vasculitis (e.g. Behcet's disease) chronic renal insufficiency, Stevens-Johnson syndrome, inflammatory pain, idiopathic sprue, cachexia, sarcoidosis, Guillain-Barŕ syndrome, uveitis, conjunctivitis, kerato conjunctivitis, otitis media, periodontal disease, pulmonary interstitial fibrosis, asthma, bronchitis, rhinitis, sinusitis, pneumoconiosis, pulmonary insufficiency syndrome, pulmonary emphysema, pulmonary fibrosis, silicosis, chronic inflammatory pulmonary disease, and chronic obstructive pulmonary disease.

For the treatment of cancer a compound of the invention may be combined with one or more of an anticancer agents. Examples of such agents can be found in Cancer Principles and Practice of Oncology by V. T. Devita and S. Heilman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved.

### Inhibition of Proliferation in REC-1 Mantle Cell Lymphoma Cells

In an embodiment, a subset of compounds of the invention is selected having any one or more of the sub-formula 1-57, 59-64, 68-86, 87b-90, 92-94b, 97b-98b, 99b, 100b-103b,104b, 108-109a, 110, 112, 114-115, 117-133b,137-139, 141-143,146-153, 155-170, 172-188, 191-207, 209-219 and 222-225.

The subset of compounds of these formulas inhibit proliferation of REC-1 mantle cell lymphoma cells with an IC₅₀ lower than 1 μM.

### Binding to Wt-BTK and Binding to Mutant-BTK

It has been found that compounds of this invention provide enhanced and prolonged binding activity to wild-type BTK. wt-BTK binding and binding kinetics were determined by measuring a binding affinity (K_{D}) and by measuring a target residence times (τ(h)).

In an embodiment, a subset of compounds of the invention is selected having any one or more of the sub-formula 1, 2, 6, 7, 8, 9, 10, 11, 17, 18, 20, 21, 24, 25, 26, 27, 28, 33, 34, 39, 40, 44, 45, 46, 47, 48, 49, 50, 51, 52, 53, 54, 56, 57, 59, 61, 68, 71, 76, 79, 83, 85b, 87b, 88b, 98b, 100b, 114, 115, 130, 131, 132, 133b, 139, 141, 146, 147, 150, 151, 152, 153, 155, 157, 162-165, 168-170, 172, 174-176, 179-187, 189, 192, 193, 195, 198-201, 203-205, 207, 210, 211, 213, 215-217 and 222-226.

The subset of compounds of these formulas provide an enhanced binding activity towards wild-type BTK as determined by binding affinity (K_{D}) using Surface Plasmon Resonance (SPR). Compounds of sub-formula 1, 2, 6, 7, 8, 9, 10, 11, 17, 18, 20, 21, 24, 25, 26, 27, 28, 33, 34, 39, 40, 44, 45, 46, 47, 48, 49, 50, 51, 52, 53, 54, 56, 57, 59, 61, 68, 71, 76, 79, 83, 85b, 87b, 88b, 98b, 100b, 114, 115, 130, 131, 132, 133b, 139, 141, 146, 147, 150, 151, 152, 153, 155, 157, 162-165, 168-170, 172, 174-176, 179-187, 189, 192, 193, 195, 198-201, 203-205, 207, 210, 211, 213, 215-217 and 222-226 showed a K_{D} (wt-BTK) value of <5 nM.

It has been found that compounds of the invention also provide binding activity to mutant forms of BTK. Mutant-BTK binding and binding kinetics were determined by measuring a binding affinity (K_{D}) and by measuring a target residence times (τ(h)) for a specific BTK mutant.

In an embodiment, a subset of compounds of the invention is selected having any one or more of the sub-formula 1, 6, 7, 8, 9, 10, 17, 20, 21, 24, 25, 26, 27, 28, 33, 34, 39, 44, 45, 46, 47, 48, 49, 50, 51, 53, 54, 56, 57, 59, 61, 68, 71, 76, 79, 83, 85b, 87b, 88b, 89b, 98b, 100b, 114, 115, 130, 131, 132, 133b, 139, 141,146, 147, 150, 151, 152, 153,155, 157,162-165, 168-170, 172,174,175,179, 183,184, 198-202,204 and 207.

The subset of compounds of these sub-formulas provide an enhanced binding activity towards BTK C481S mutant as determined by binding affinity (K_{D}) using Surface Plasmon Resonance (SPR). Compounds of sub-formula 1, 6, 7, 8, 9, 10, 17, 20, 21, 24, 25, 26, 27, 28, 33, 34, 39, 44, 45, 46, 47, 48, 49, 50, 51, 53, 54, 56, 57, 59, 61, 68, 71, 76, 79, 83, 85b, 87b, 88b, 89b, 98b, 100b, 114, 115, 130, 131, 132, 133b, 139, 141, 146, 147, 150, 151, 152, 153, 155,157,162-165,168-170,172,174,175,179,183,184,198-202,204 and 207 showed a K_{D} (BTK C481S) value of <5 nM.

In an embodiment, a subset of compounds of the invention is selected having any one or more of the sub-formula 1, 2, 6, 7, 8, 9, 10, 11, 17, 18, 20, 21, 24, 25, 26, 27, 28, 33, 34, 39, 40, 44, 45, 46, 47, 48, 49, 50, 51, 53, 54, 56, 57, 59, 61, 68, 71, 76, 79, 83, 87b, 88b, 89b, 98b, 100b, 115, 130, 131,132, 133b, 139, 141,146, 147,150, 152,153, 155, 157, 162-165,168-170,172 and 174-176.

The subset of compounds of these formulas provide an enhanced binding activity towards BTK T316A mutant as determined by binding affinity (K_{D}) using Surface Plasmon Resonance (SPR). Compounds of sub-formula 1, 2, 6, 7, 8, 9, 10, 11, 17, 18, 20, 21, 24, 25, 26, 27, 28, 33, 34, 39, 40, 44, 45, 46, 47, 48, 49, 50, 51, 53, 54, 56, 57, 59, 61, 68, 71, 76, 79, 83, 87b, 88b, 89b, 98b, 100b, 115, 130, 131, 132, 133b, 139, 141, 146, 147, 150, 152, 153, 155, 157,162-165, 168-170, 172 and 174-176 showed a K_{D} (BTK T316A) value of <5 nM.

In an embodiment, a subset of compounds of the invention is selected having any one or more of the sub-formula 1, 2, 6, 7, 10, 11, 17, 18, 20, 21, 24, 25, 26, 27, 28, 33, 34, 39, 40, 44, 45, 46, 47, 48, 49, 50, 51, 57, 61, 68, 71, 79, 83, 130, 131, 132, 133b, 141, 147, 150, 151, 152, 153, 155,157, 162-165,168-170, 172,174-176, 179, 180, 182-189, 192, 193, 198 205, 207, 210, 213, 215-217 and 222-226 and sub-formula 8, 9, 50, 51, 52, 53, 56, 59, 76, 85b, 87b, 88b, 89b, 98b, 100b, 114, 115, 139, 146, 156 and 211. In a particular embodiment, the subset of compounds comprises any one or more of the sub-formula 1, 2, 6, 7, 10, 11, 17, 18, 20, 21, 24, 25, 26, 27, 28, 33, 34, 39, 40, 44, 45, 46, 47, 48, 49, 54, 57, 61, 68, 71, 79, 83, 130, 131,132, 133b, 141, 147,150,151,152, 153,155,157, 162-165, 168-170, 172, 174-176, 179, 180, 182-189, 192, 193, 198-205, 207, 210, 213, 215-217 and 222-226.

The subset of compounds of these formulas provide an enhanced binding activity towards BTK T474I mutant as determined by binding affinity (K_{D}) using Surface Plasmon Resonance (SPR). Compounds of examples 8, 9, 50, 51, 52, 53, 56, 59, 76, 85b, 87b, 88b, 89b, 98b, 100b, 114, 115, 139, 146, 156 and 211 showed a K_{D} (BTK T474I) value>10 nM and <50 nM, and compounds of sub-formula 1, 2, 6, 7, 10, 11, 17, 18, 20, 21, 24, 25, 26, 27, 28, 33, 34, 39, 40, 44, 45, 46, 47, 48, 49, 54, 57, 61, 68, 71, 79, 83, 130, 131, 132, 133b, 141, 147, 150, 151, 152,153,155, 157,162-165,168-170, 172, 174-176, 179,180, 182-189,192, 193, 198-205, 207, 210, 213, 215-217 and 222-226 showed a K_{D} (BTK T474I) value<10 nM.

In an embodiment, a subset of compounds of the invention is selected having any one or more of the sub-formula 6, 7, 8, 9, 10, 11, 17, 18, 20, 21, 24, 25, 26, 27, 28, 33, 34, 39, 40, 44, 45, 46, 47, 48, 49, 50, 51, 52, 53, 54, 56, 57, 59, 61, 68, 71, 76, 79, 83, 85b, 87b, 88b, 98b, 100b, 115, 130, 131, 132, 133b, 139, 141, 146, 147, 150, 151, 152, 153, 155, 156, 157, 162-165,168-170,172, 174-176,179-181,183 and 187.

The subset of compounds of these formulas provide an enhanced binding activity towards BTK T474S mutant as determined by binding affinity (K_{D}) using Surface Plasmon Resonance (SPR). Compounds of sub-formula 6, 7, 8, 9, 10, 11, 17, 18, 20, 21, 24, 25, 26, 27, 28, 33, 34, 39, 40, 44, 45, 46, 47, 48, 49, 50, 51, 52, 53, 54, 56, 57, 59, 61, 68, 71, 76, 79, 83, 85b, 87b, 88b, 98b, 100b, 115, 130, 131, 132, 133b, 139, 141, 146, 147, 150, 151, 152, 153, 155, 156, 157, 162-165, 168-170, 172, 174-176, 179-181, 183 and 187 showed a K_{D} (BTK T474S) value of <5 nM.

These compounds are illustrated above.

### Activity Towards Wt-BTK and Towards Mutant-BTK

In a preferred embodiment, a subset of compounds of the invention is selected having any one or more of the sub-formula 10, 11, 25, 26, 33, 34, 40, 44, 45, 46, 162, 163, 164, 165, 166, 168, 169, 170, 174, 179,182,183, 184,185,186,187, 188,192,193, 196, 198,199,200, 201, 204, 205, 210, 213, 216, 217, 222 and 224.

The subset of compounds of these formulas provide enhanced binding activity towards wt-BTK and towards mutant-BTK and provide a long target residence time towards wt-BTK and towards mutant-BTK.

### Routes of Administration

Suitable routes of administration may, for example, include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

### Composition/Formulation

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated.

Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds can be formulated for parenteral administration by injection, e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection).

Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

### Synthesis of compounds

The compounds of the present invention can be prepared by methods well known in the art of organic chemistry. See, for example, J. March, ‘Advanced Organic Chemistry’ 4^{th} Edition, John Wiley and Sons. During synthetic sequences it may be necessary and/or desirable to protect sensitive or reactive groups on any of the molecules concerned. This is achieved by means of conventional protecting groups, such as those described in T. W. Greene and P. G. M. Wutts ‘Protective Groups in Organic Synthesis’ 3^{rd} Edition, John Wiley and Sons, 1999. The protective groups are optionally removed at a convenient subsequent stage using methods well known in the art.

The products of the reactions are optionally isolated and purified, if desired, using conventional techniques, including but not limited to, filtration, distillation, crystallization, chromatography and the like. Such materials are optionally characterized using conventional means, including physical constants and spectral data.

Compounds of any one of Formula I-a to I-h, wherein R¹ to R⁴ and W, V and U have the previously defined meanings, can be prepared by the general synthetic route shown in any one of scheme I-XII. In particular examples, compounds of any one of Formula I-a and I-b are prepared by the general synthetic route shown in any one of scheme I-XII.

Scheme I and II shows a general synthetic route in relation to an exemplary compound of Formula I-a.

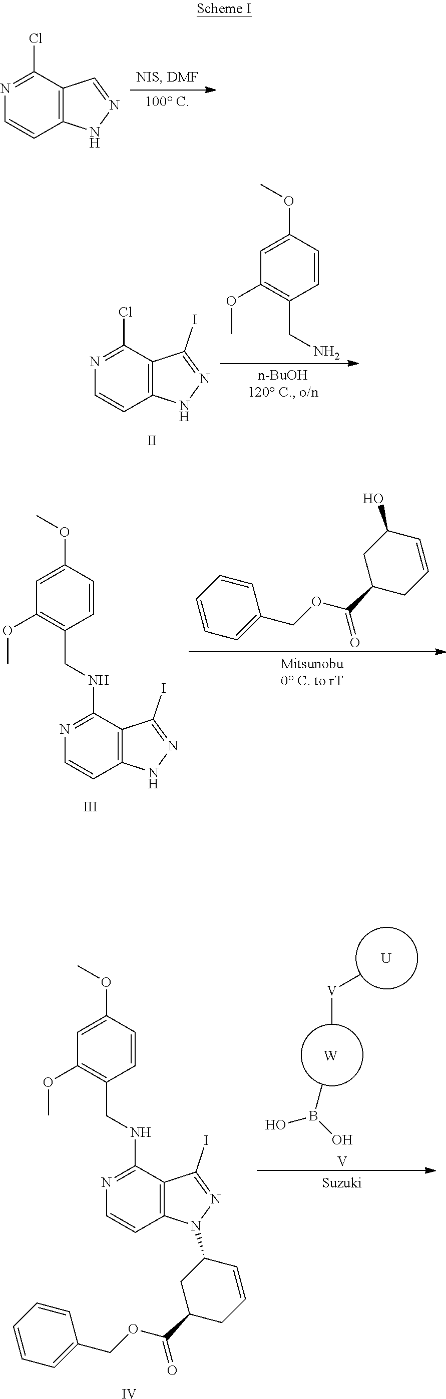

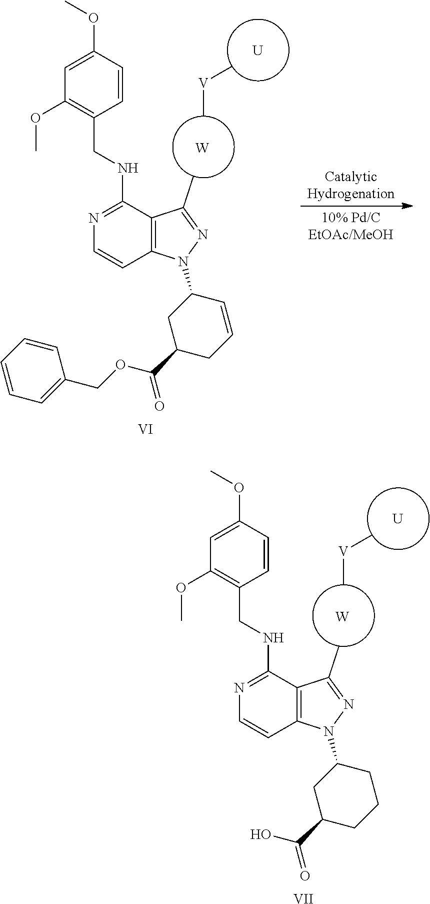

4-Chloro-3-iodo-1 H-pyrazolo[4,3-c]pyridine II can be prepared from commercially available 4-chloro-1 H-pyrazolo[4,3-c]pyridine using N-iodosuccinimide in a solvent such as DMF at elevated temperatures. The resulting product can then be reacted with 2,4-dimethoxybenzylamine in an appropriate solvent like n-butanol, isopropanol or 2-pentanol at high temperatures to obtain N-[(2,4-dimethoxyphenyl)methyl]-3-iodo-1 H-pyrazolo[4,3-c]pyridin-4-amine III. Compound IV can, subsequently, be prepared from compound 11 and benzyl(1R,5R)— 5-hydroxycyclohex-3-ene-1-carboxylate using Mitsunobu conditions, for example DIAD/triphenylphosphine in THE at 0° C. Compound VI can be prepared from compound IV using an appropriate boronic acid or pinacolester (V), in the presence of a suitable palladium catalyst system, for example bis(diphenylphosphine)palladium(0) palladium(II)chloride complex or tetrakis(triphenylphosphine)palladium(0) in the presence of an inorganic base like potassium carbonate, cesium carbonate or potassium phosphate in a suitable solvent system like combinations of dioxane and water. Reduction of the double bond and deprotection of the benzylester can be accomplished by catalytic hydrogenation in the presence of a suitable catalyst system and solvent, for example palladium on charcoal in ethyl acetate and methanol to provide compounds of Formula VII.

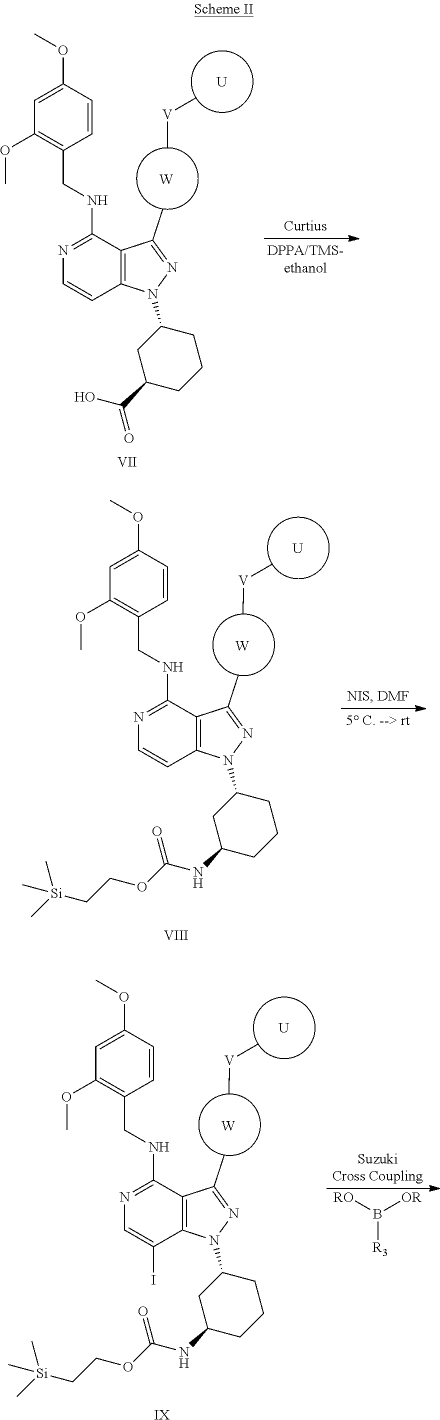

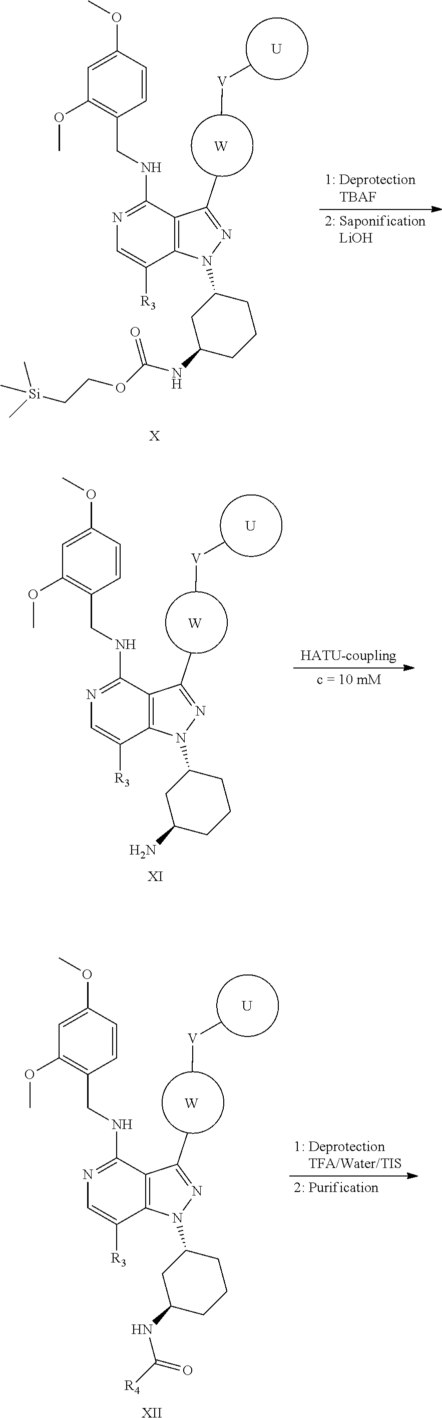

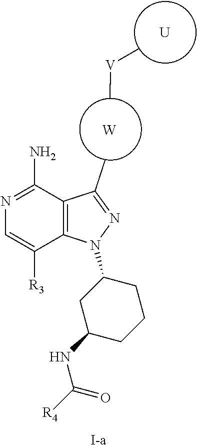

Compounds of Formula VIII can be prepared from derivatives VII using diphenylphosphorylazide in toluene or THF and a suitable alcohol such as trimethylsilylethanol, benzylalcohol or tert-butanol. Subsequent halogenation can be performed using N-bromosuccinimide or N-iodosuccinimide in a suitable solvent like DCM or DMF at appropriate temperature to obtain compounds of Formula IX. Compounds of Formula X can be prepared from compound IX using an appropriate boronic acid or pinacolester, in the presence of a suitable palladium catalyst system, for example CataCXium® A Pd G3 or bis(diphenylphos-phine)palladium(0) palladium(II)chloride complex in the presence of an inorganic base like potassium carbonate, cesium carbonate or potassium phosphate in a suitable solvent system like combinations of dioxane and water. Derivatives of Formula XI can be prepared from derivatives of Formula X after deprotection of the amino function with TBAF or a strong acid like TFA and subsequent carboxyl acid deprotection using a suitable inorganic base like lithium hydroxide or sodium hydroxide. Macrocyclization towards compounds of Formula XII can be accomplished with an appropriate couplings reagent such as HATU of EDCI.HCl in a suitable solvent like DMF at appropriate temperature. Finally conversion of compounds of Formula XII to compounds with Formula I-a can be accomplished using strong acids like HCl or TFA in the presence of water and a suitable cation scavenger like triisopropylsilane (TIS) at appropriate temperature.

Alternatively compounds of Formula I-a to I-h, wherein R¹ to R⁴ and W, V and U have the previously defined meanings, can be prepared by the general synthetic route shown in scheme III. Scheme III shows a general synthetic route in relation to an exemplary compound of Formula I-a.

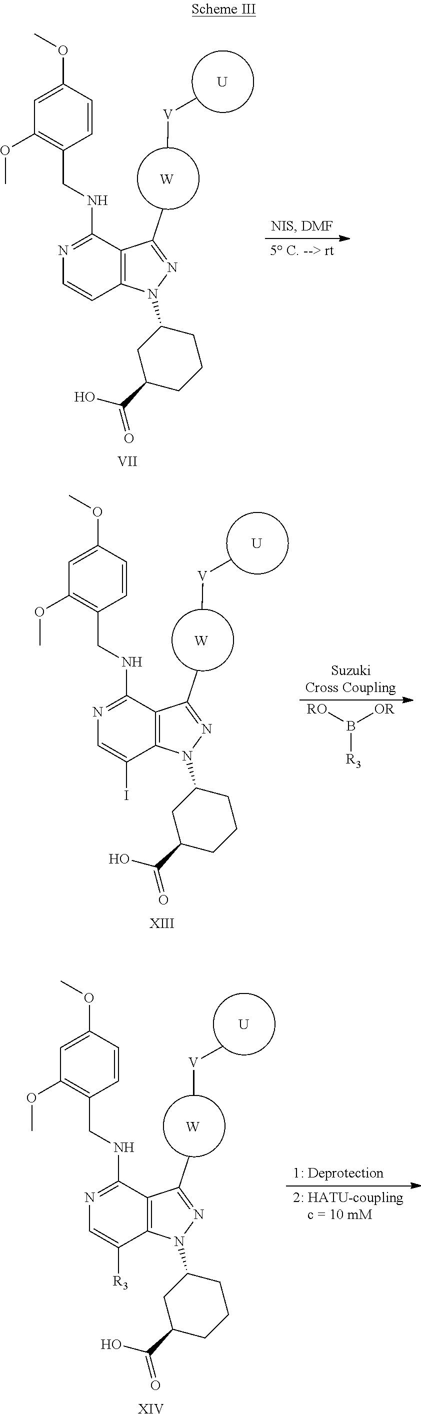

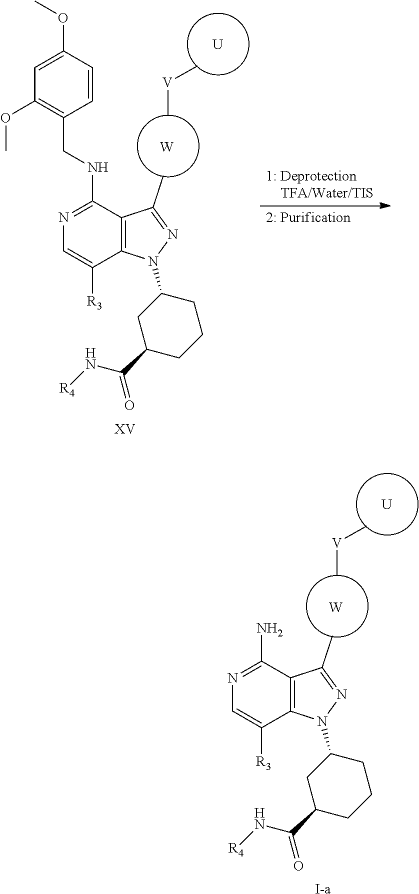

Halogenation of compounds of Formula VII can be performed using N-bromosuccinimide or N-iodosuccinimide in a suitable solvent like DCM or DMF at appropriate temperature to obtain compounds of Formula XIII. Compounds of Formula XIV can be prepared from compound XIII using an appropriate boronic acid or pinacolester, in the presence of a suitable palladium catalyst system, for example CataCXium® A Pd G3 or bis(diphenylphos-phine)palladium(0) palladium(II)chloride complex in the presence of an inorganic base like potassium carbonate, cesium carbonate or potassium phosphate in a suitable solvent system like combinations of dioxane and water. Derivatives of Formula XV can be prepared from derivatives of Formula XIV after carboxyl acid deprotection using a suitable inorganic base like lithium hydroxide or sodium hydroxide. Macrocyclization towards compounds of Formula XV can be accomplished with an appropriate couplings reagent such as HATU of EDCI.HCl in a suitable solvent like DMF at appropriate temperature. Finally conversion of compounds of Formula XV to compounds with Formula I-a can be accomplished using strong acids like HCl or TFA in the presence of water and TIS at appropriate temperature.

Another route to obtain compounds of Formula I-a to I-h, wherein R¹ to R⁴ and W, V and U have the previously defined meanings, is depicted in the general synthetic route shown in scheme IV. Scheme IV shows a general synthetic route in relation to an exemplary compound of Formula I-a.

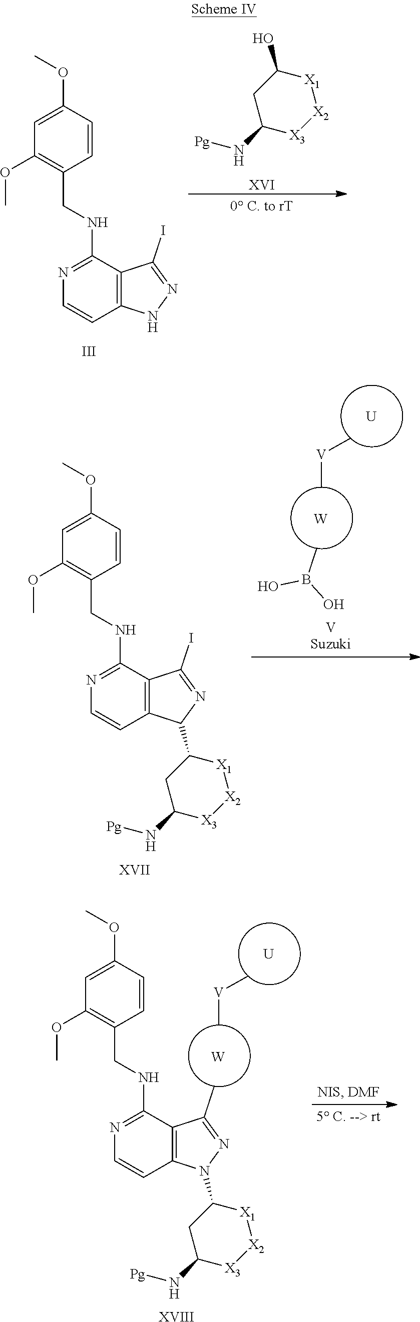

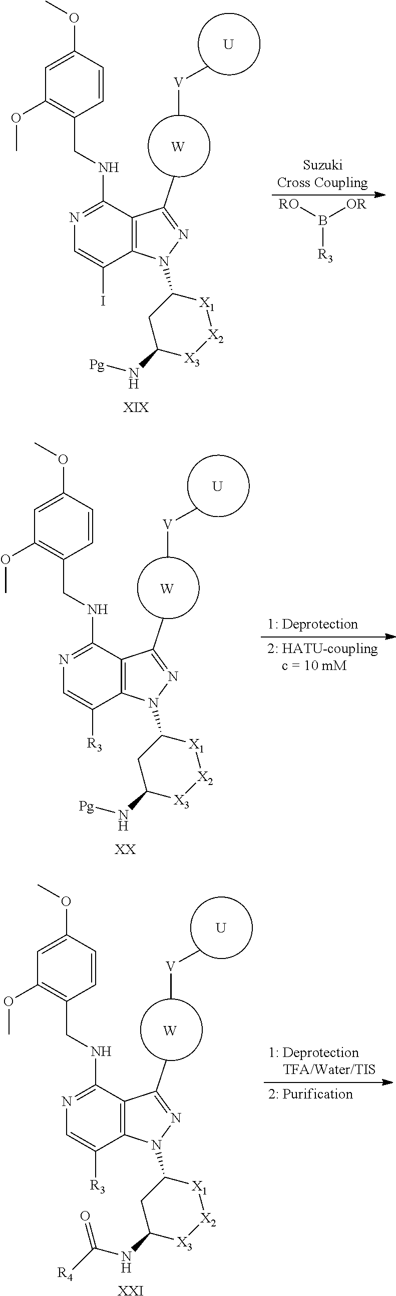

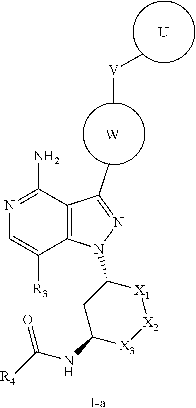

Compounds of Formula XVII can be prepared from compound III and amino-protected (chiral) aminoalcohols (XVI) using Mitsunobu conditions, for example DIAD/triphenylphosphine in THE at 0° C. Alternatively compounds of Formula XVII can be obtained after activation of the alcohol with for example tosylchloride or mesylchloride to perform a substitution reaction in appropriate solvents such as DMF in the presence of an inorganic base like cesium carbonate or potassium carbonate. Compounds of Formula XVIII can be prepared from compound XVII using an appropriate boronic acid or pinacolester (V), in the presence of a suitable palladium catalyst system, for example bis(diphenylphosphine)palladium(0) palladium(II)chloride complex or tetrakis(triphenylphos-phine)palladium(0) in the presence of an inorganic base like potassium carbonate, cesium carbonate or potassium phosphate in a suitable solvent system like combinations of dioxane and water. Subsequent halogenation of compounds of Formula XVIII can be performed using N-bromosuccinimide or N-iodosuccinimide in a suitable solvent like DCM or DMF at appropriate temperature to obtain compounds of Formula XIX. Compounds of Formula XX can be prepared from compound XIX using an appropriate boronic acid or pinacolester, in the presence of a suitable palladium catalyst system, for example CataCXium® A Pd G3 or bis(diphenylphosphine)palladium(0) palladium(II)chloride complex in the presence of a inorganic base like potassium carbonate, cesium carbonate or potassium phosphate in a suitable solvent system like combinations of dioxane and water. Derivatives of Formula XXI can be prepared from derivatives of Formula XX after carboxyl acid deprotection using a suitable inorganic base like lithium hydroxide or sodium hydroxide, subsequent macrocyclization towards compounds of Formula XXI can be accomplished with an appropriate couplings reagent such as HATU of EDCI.HCl in a suitable solvent like DMF at appropriate temperature. Subsequent removal of the DMB-group using methods known by skilled organic chemists, such as TFA containing triethylsilane provides compounds of Formula I-a.

Yet another route to obtain compounds of Formula I-a to I-h, wherein R¹ to R⁴ and W, V and U have the previously defined meanings, is depicted in the general synthetic route shown in scheme V. Scheme V shows a general synthetic route in relation to an exemplary compound of Formula I-a.

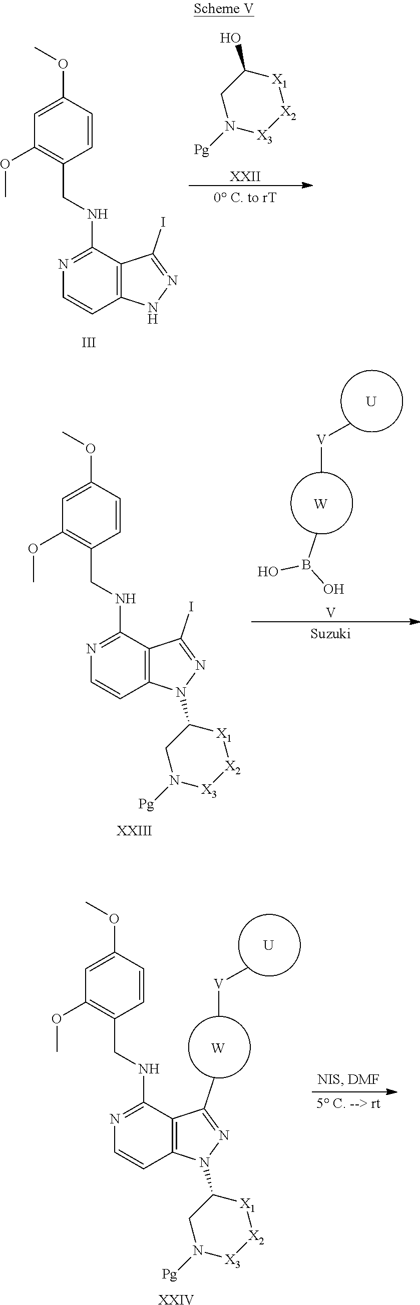

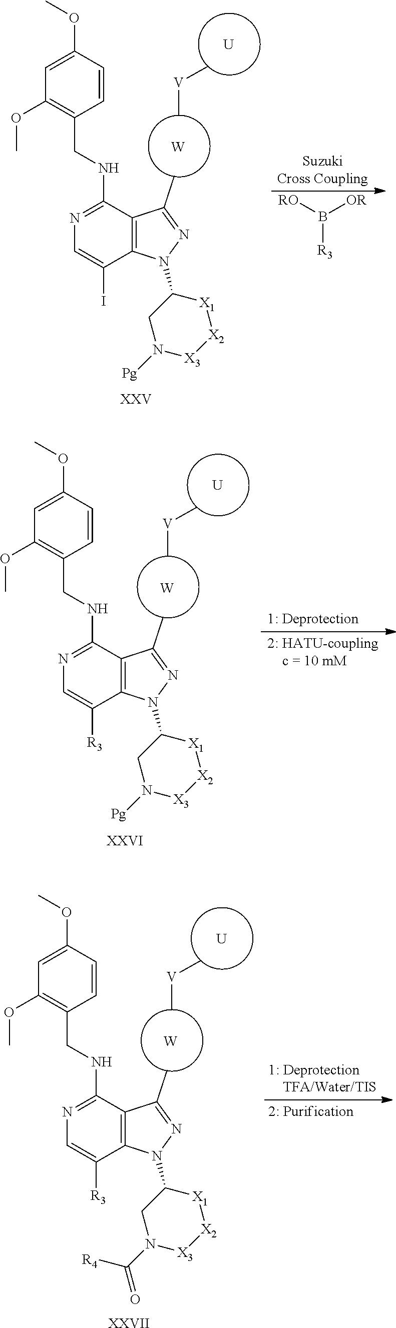

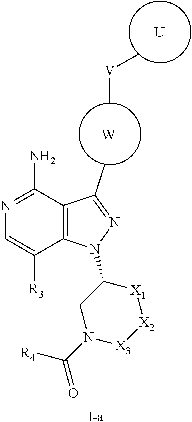

Compounds of Formula XXIII can be prepared from compound III and amino-protected (chiral) aminoalcohols (XXII) using Mitsunobu conditions, for example DIAD/triphenylphosphine in THE at 0° C. Alternatively, compounds of Formula XXIII can be obtained after activation of the alcohol with for example tosylchloride or mesylchloride to perform a substitution reaction in appropriate solvents such as DMF in the presence of an inorganic base like cesium carbonate or potassium carbonate. Compounds of Formula XXIV can be prepared from compound XXIII using an appropriate boronic acid or pinacolester (V), in the presence of a suitable palladium catalyst system, for example bis(diphenylphosphine)palladium(0) palladium(II)chloride complex or tetrakis(triphenylphosphine)palladium(0) in the presence of an inorganic base like potassium carbonate, cesium carbonate or potassium phosphate in a suitable solvent system like combinations of dioxane and water. Subsequent halogenation of compounds of Formula XXIV can be performed using N-bromosuccinimide or N-iodosuccinimide in a suitable solvent like DCM or DMF at appropriate temperature to obtain compounds of Formula XXV. Compounds of Formula XXVI can be prepared from compound XXV using an appropriate boronic acid or pinacolester, in the presence of a suitable palladium catalyst system, for example CataCXium® A Pd G3 or bis(diphenylphosphine)palladium(0) palladium(II)chloride complex in the presence of an inorganic base like potassium carbonate, cesium carbonate or potassium phosphate in a suitable solvent system like combinations of dioxane and water. Derivatives of Formula XXVII can be prepared from derivatives of Formula XXVI after deprotection of the amine group with TBAF or a strong acid like TFA and subsequent carboxyl acid deprotection using a suitable inorganic base like lithium hydroxide or sodium hydroxide, following macrocyclization towards compounds of Formula XXVII can be accomplished with an appropriate coupling-reagent such as HATU of EDCI.HCl in a suitable solvent like DMF at appropriate temperature. Subsequent removal of the DMB-group using methods known by skilled organic chemists, such as TFA containing TIS provides compounds of Formula I-a.

Alternatively, compounds of Formula I-a to I-h, wherein R¹ to R⁴ and W, V and U have the previously defined meanings, is depicted in the general synthetic route shown in scheme VI. Scheme VI shows a general synthetic route in relation to an exemplary compound of Formula I-a.

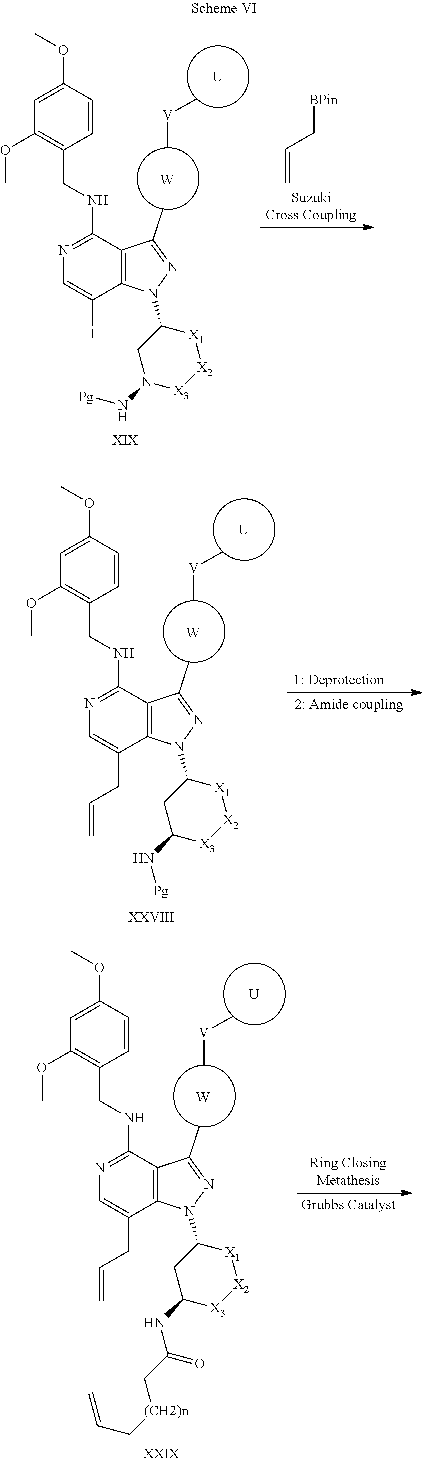

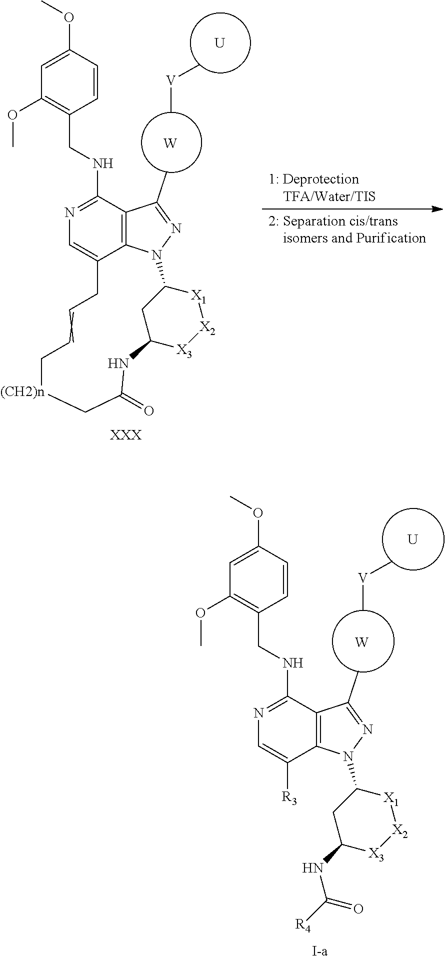

Compounds of Formula XXVIII can be prepared from compound XIX using 2-allyl-4,4,5,5-tetramethyl-1,3,2-dioxaborolane or potassium allyltrifluoroborate, in the presence of a suitable palladium catalyst system, for example CataCXium® A Pd G3 or bis(diphenylphosphine)palladium(0) palladium(II)chloride complex in the presence of an inorganic base like potassium carbonate, cesium carbonate or potassium phosphate in a suitable solvent system like combinations of dioxane and water. Derotection of derivatives of Formula XXVIII can accomplished using methods known by those skilled in the art, such as TBAF of a strong acid like TFA. Subsequently compounds of Formula XXIX can be obtained with terminal alkenes containing an acid functionality, using an appropriate coupling-reagent such as HATU of EDCI.HCl in a suitable solvent like DMF, THE or DCM at appropriate temperature. Ring closing metathesis towards compounds of Formula XXX can be accomplished with an appropriate Grubbs catalyst, molybdene or ruthenium catalyst in a suitable solvent like DCM or toluene at appropriate temperature. Subsequent removal of the DMB-group using methods known by skilled organic chemists, such as TFA containing triethylsilane provides isomeric mixtures of compounds of Formula I-a. The thus obtained mixtures of cis/trans isomers of Formula I-a could be separated using chromatographic techniques such as HPLC to obtain compounds of Formula I-a.

Another route to obtain compounds of Formula I-a to I-h, wherein R¹ to R⁴ and W, V and U have the previously defined meanings, is depicted in the general synthetic route shown in scheme VII. Scheme VII shows a general synthetic route in relation to an exemplary compound of Formula I-a.

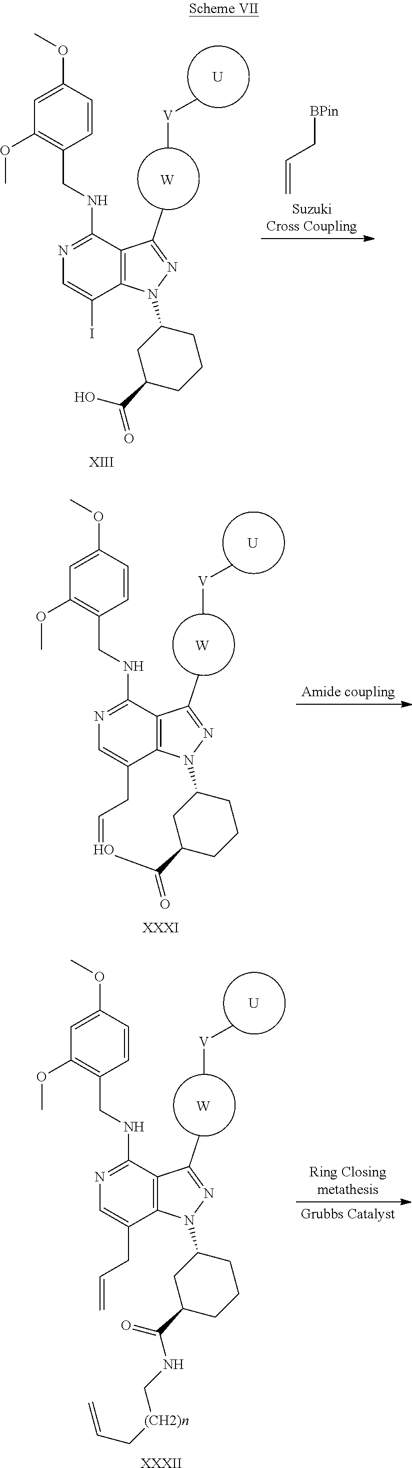

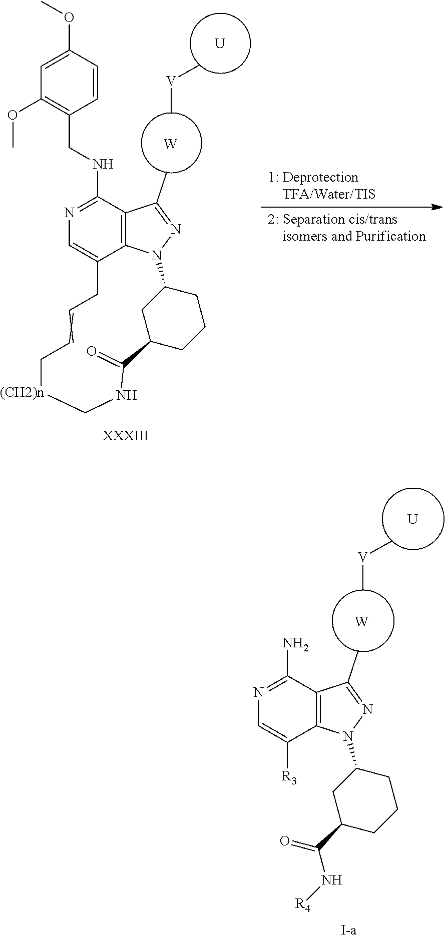

Compounds of Formula XXXI can be prepared from compound XIII using 2-allyl-4,4,5,5-tetramethyl-1,3,2-dioxaborolane or potassium allyltrifluoroborate, in the presence of a suitable palladium catalyst system, for example CataCXium® A Pd G3 or bis(diphenylphosphine)palladium(0) palladium(II)chloride complex in the presence of an inorganic base like potassium carbonate, cesium carbonate or potassium phosphate in a suitable solvent system like combinations of dioxane and water. Subsequently compounds of Formula XXXII can be obtained from terminal alkenes containing an amine functionality, an appropriate coupling-reagent such as HATU of EDCI.HCl in a suitable solvent like DMF, THE or DCM at appropriate temperature. Ring closing metathesis towards compounds of Formula XXXIII can be accomplished with an appropriate Grubbs catalyst, molybdene or ruthenium catalyst in a suitable solvent like DCM or toluene at appropriate temperature. Subsequent removal of the DMB-group using methods known by skilled organic chemists, such as TFA containing triethylsilane provides isomeric mixtures of compounds of Formula I-a. The thus obtained mixtures of cis/trans isomers of Formula I-a could be separated using chromatographic techniques such as HPLC to obtain compounds of Formula I-a.

Yet another route to obtain compounds of Formula I-a to I-h, wherein R¹ to R⁴ and W, V and U have the previously defined meanings, is depicted in the general synthetic route shown in scheme VIII. Scheme VIII shows a general synthetic route in relation to an exemplary compound of Formula 1-b.

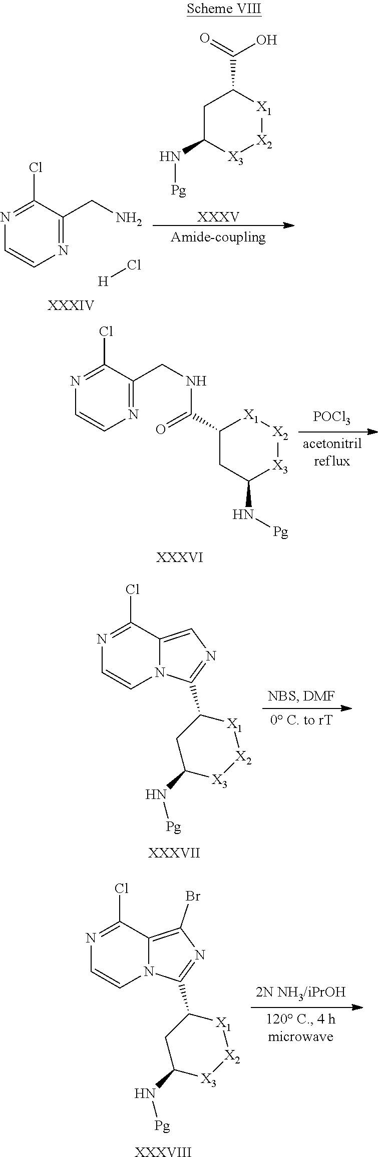

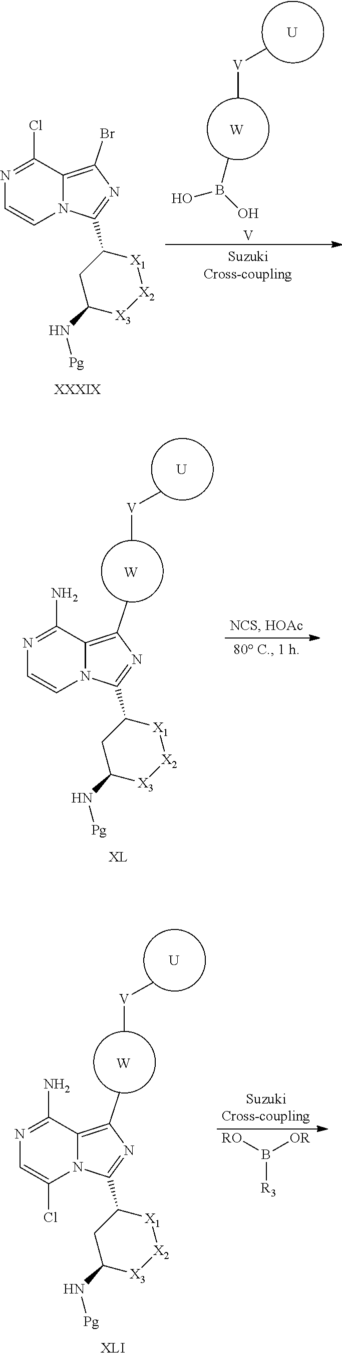

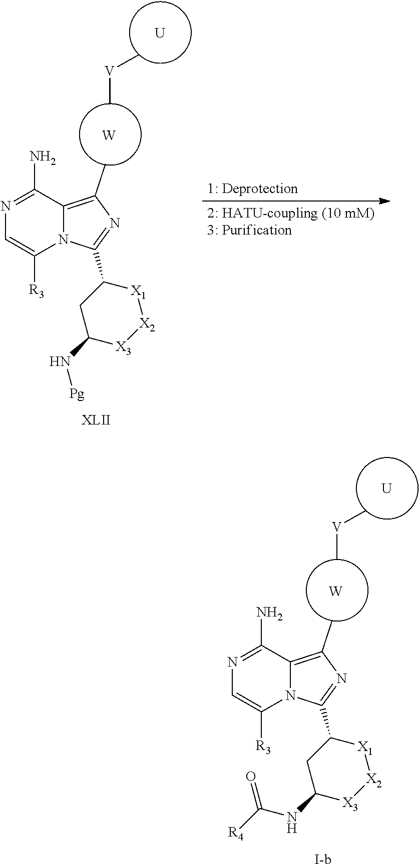

The reaction of (3-chloropyrazine-2-yl)methanamine.hydrochloride (XXXIV) can be carried out with an appropriately amine protected amino acid (XXXV) in a solvent such as DMF, THE or DCM in the presence of a base such as DIPEA, N-methylmorpholine, 4-DMAP or triethylamine and in the presence of a coupling reagent such as PyBOP, TBTU, EDCI or HATU to form compounds of Formula XXXVI. Cyclisation of chloropyrazine of with Formula XXXVI can be performed using condensation reagents like phosphorus oxychloride under heating conditions to provide compounds of Formula XXXVII. Subsequent bromination can be accomplished using bromine or N-bromosuccinimide in a suitable solvent like DCM or DMF at appropriate temperature to obtain compounds of Formula XXXVIII. 8-Aminoimidazo[1,5a]pyrazine derivatives XXXIX can be prepared from compounds of Formula XXXVIII using ammonia(gas) in isopropanol at elevated temperature in a pressure vessel or microwave (>4 atm.) Compounds of Formula XL can be prepared from compound XXXIX using an appropriate boronic acid or pinacolester (V), in the presence of a suitable palladium catalyst system, for example bis(diphenylphosphine)palladium(0) palladium(II)chloride complex or tetrakis(triphenylphos-phine)palladium(0) in the presence of an inorganic base like potassium carbonate, cesium carbonate or potassium phosphate in a suitable solvent system like combinations of dioxane and water. Subsequent halogenation of compounds of Formula XL can be performed using N-chlorosuccinimide in a suitable solvent like acetic acid at appropriate temperature to obtain compounds of Formula XLI. Compounds of Formula XLII can be prepared from compound XLI using an appropriate boronic acid or pinacolester, in the presence of a suitable palladium catalyst system, for example CataCXium® A Pd G3 or bis(diphenylphosphine)palladium(0) palladium(II)chloride complex in the presence of an inorganic base like potassium carbonate, cesium carbonate or potassium phosphate in a suitable solvent system like combinations of dioxane and water. Derivatives of Formula I can be prepared from derivatives of Formula XLII after deprotection of the amine group with TBAF or a strong acid like TFA and subsequent carboxyl acid deprotection using a suitable inorganic base like lithium hydroxide or sodium hydroxide. Following macrocyclization towards compounds of Formula 1-b can be accomplished with an appropriate coupling-reagent such as HATU of EDCI.HCl in a suitable solvent like DMF at appropriate temperature. Subsequent removal of the DMB-group using methods known by skilled organic chemists, such as TFA containing triethylsilane to obtain compounds of Formula 1-b.

Yet another route to obtain compounds of Formula I-a to I-h, wherein R¹ to R⁴ and W, V and U have the previously defined meanings, is depicted in the general synthetic route shown in scheme IX. Scheme IX shows a general synthetic route in relation to an exemplary compound of Formula I-b.

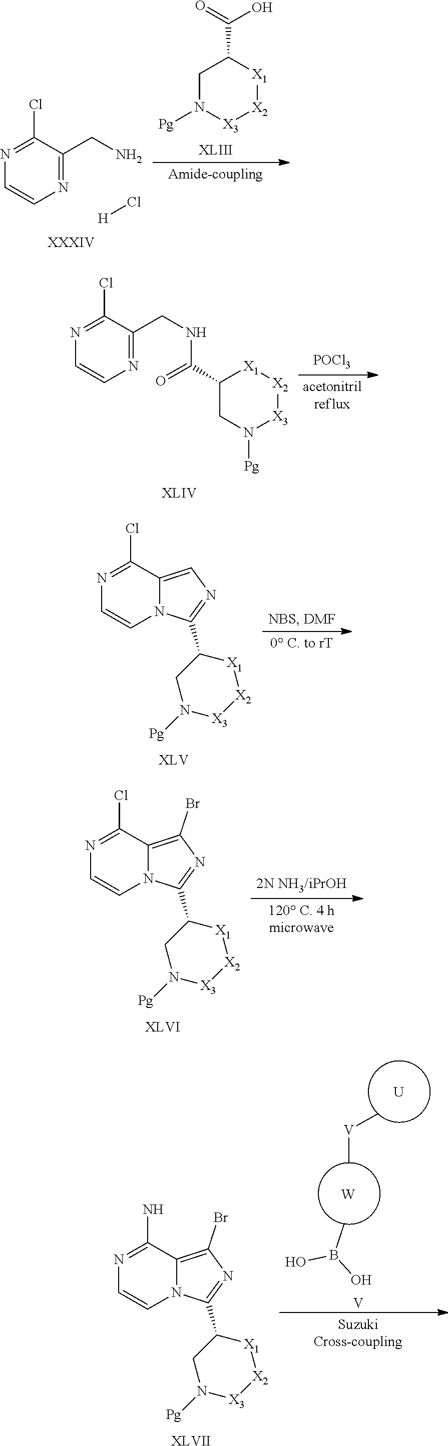

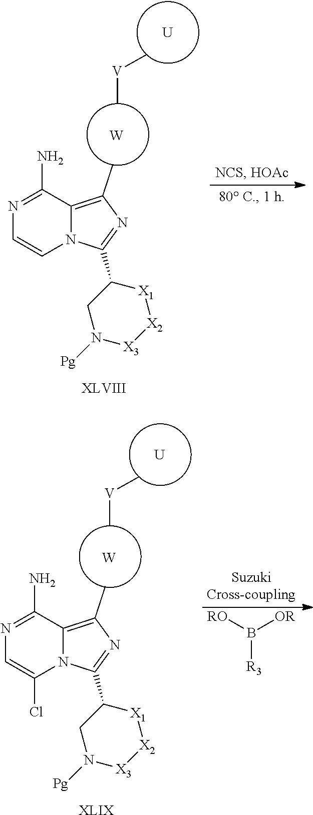

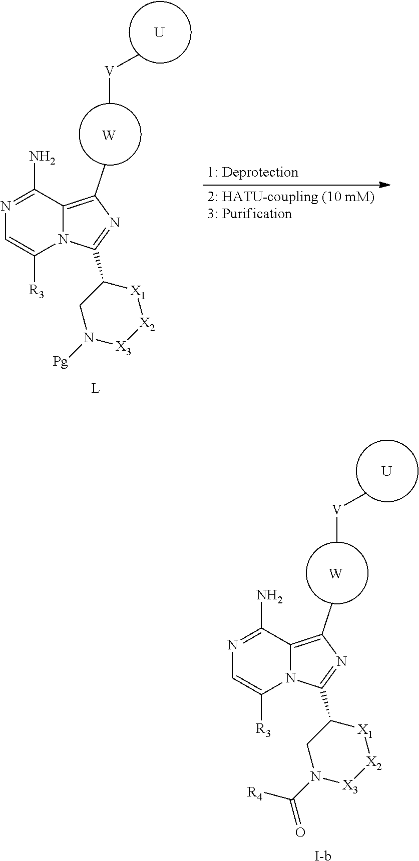

The reaction of (3-chloropyrazine-2-yl)methanamine.hydrochloride (XXXIV) can be carried out with an appropriately amine protected amino acid (XLIII) in a solvent such as DMF, THE or DCM in the presence of a base such as DIPEA, N-methylmorpholine, 4-DMAP or triethylamine and in the presence of a coupling reagent such as PyBOP, TBTU, EDCI or HATU to form compounds of Formula XLIV. Cyclisation of chloropyrazine of Formula XLIV can be performed using condensation reagents like phophorus oxychloride under heating conditions to provide compounds of Formula XLV. Subsequent bromination can be accomplished using bromine or N-bromosuccinimide in a suitable solvent like DCM or DMF at appropriate temperature to obtain compounds of Formula XLVI. 8-Aminoimidazo[1,5a]pyrazine derivatives XLVII can be prepared from compounds of Formula XLVI using ammonia (gas) in isopropanol at elevated temperature in a pressure vessel or microwave (>4 atm.) Compounds of Formula XLVIII can be prepared from compound XLVII using an appropriate boronic acid or pinacolester (V), in the presence of a suitable palladium catalyst system, for example bis(diphenylphosphine)palladium(0) palladium(II)chloride complex or tetrakis(triphenylphos-phine)palladium(0) in the presence of an inorganic base like potassium carbonate, cesium carbonate or potassium phosphate in a suitable solvent system like combinations of dioxane and water. Subsequent halogenation of compounds of Formula XLVIII can be performed using N-chlorosuccinimide in a suitable solvent like acetic acid at appropriate temperature to obtain compounds of Formula XLIX. Compounds of Formula L can be prepared from compound XLIX using an appropriate boronic acid or pinacolester, in the presence of a suitable palladium catalyst system, for example CataCXium® A Pd G3 or bis(diphenylphosphine)palladium(0) palladium(II)chloride complex in the presence of an inorganic base like potassium carbonate, cesium carbonate or potassium phosphate in a suitable solvent system like combinations of dioxane and water. Derivatives of Formula I can be prepared from derivatives of Formula L after deprotection of the amine group with TBAF or a strong acid like TFA and subsequent carboxyl acid deprotection using a suitable inorganic base like lithium hydroxide or sodium hydroxide.

Following macrocyclization towards compounds of Formula 1-b can be accomplished with an appropriate coupling-reagent such as HATU of EDCI.HCl in a suitable solvent like DMF at appropriate temperature. Subsequent removal of the DMB-group using methods known by skilled organic chemists, such as TFA containing triethylsilane to obtain compounds of Formula I-b.

Yet another route to obtain compounds of Formula I-a to I-h, wherein R¹ to R⁴ and W, V and U have the previously defined meanings, is depicted in the general synthetic route shown in scheme X. Scheme X shows a general synthetic route in relation to an exemplary compound of Formula I-b.

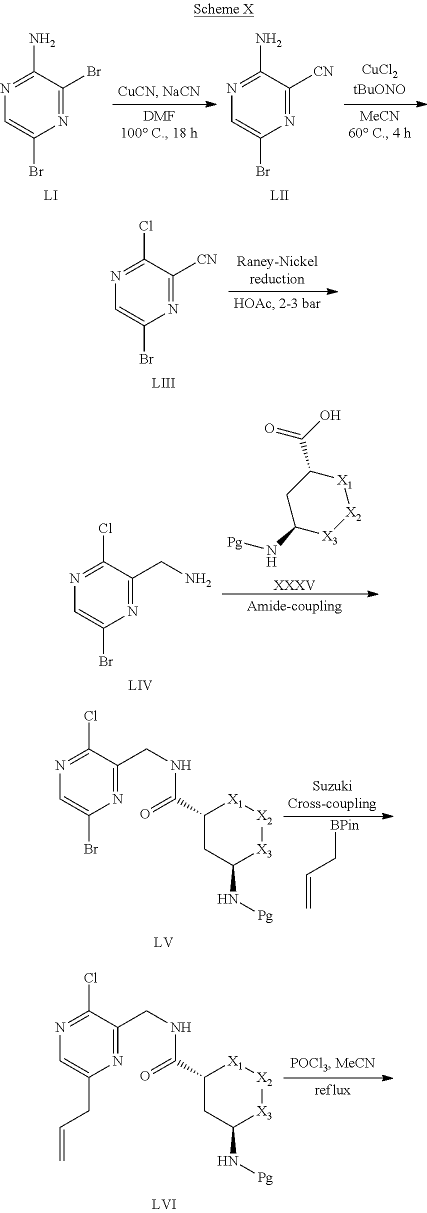

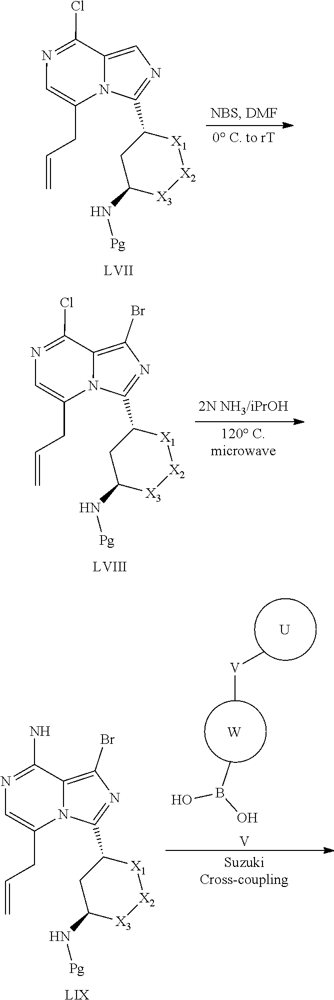

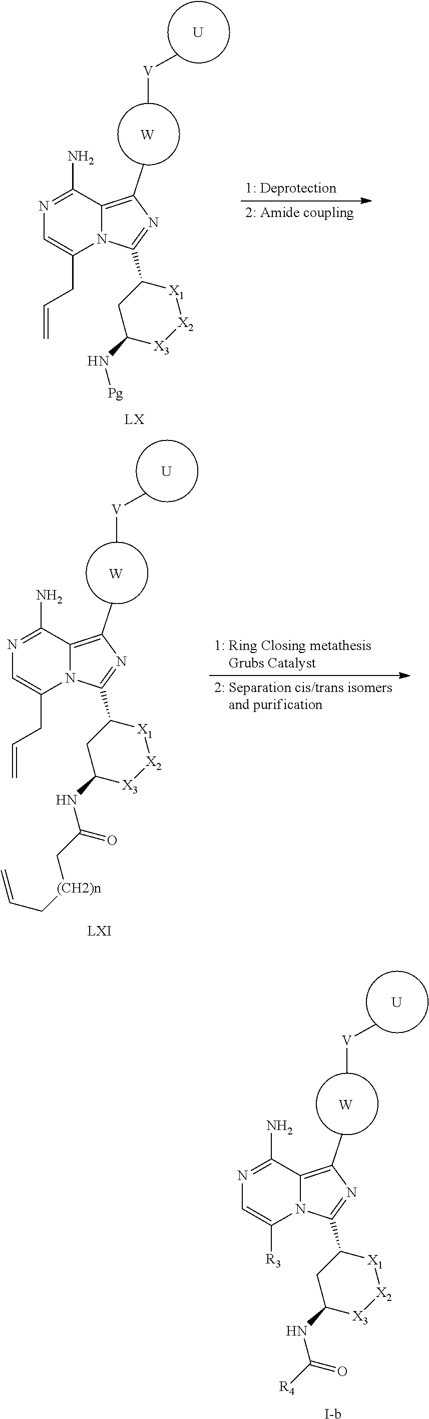

3-Amino-6-bromo-pyrazine-2-carbonitrile (LII) can be prepared from commercial available 2-amino-3,5-dibromo pyrazine (LI) using copper cyanide and sodium cyanide in DMF at elevated temperature. The resulting product can then be converted to 6-bromo-3-chloro-pyrazine-2-carbonitrile (LIII) under Sandmeyer conditions with copper chloride, tert-butylnitrite in an appropriate solvent such as acetonitrile under heating. Reduction of derivative LIII can be accomplished by hydrogenation under elevated pressure in the presence of a suitable catalyst system and solvent, for example Raney-Nickel to provide (6-bromo-3-chloro-pyrazin-2-yl)methanamine (LIV). This can then be reacted with an appropriately amine protected amino acid (XXXV) in a solvent such as DMF, THE or DCM in the presence of a base such as DIPEA, N-methylmorpholine, 4-DMAP or triethylamine and in the presence of a coupling reagent such as PyBOP, TBTU, EDCI or HATU to form compounds of Formula LV. Compounds of Formula LVI can be prepared from compound LV using 2-allyl-4,4,5,5-tetramethyl-1,3,2-dioxaborolane or potassium allyltrifluoroborate, in the presence of a suitable palladium catalyst system, for example CataCXium® A Pd G3 or bis(diphenylphosphine)palladium(0) palladium(II)chloride complex in the presence of an inorganic base like potassium carbonate, cesium carbonate or potassium phosphate in a suitable solvent system like combinations of dioxane and water. Cyclisation chloropyrazine of Formula LVI can be performed using condensation reagents like phosphorus oxychloride under heating conditions to provide compounds of Formula LVII. Subsequent bromination can be accomplished using bromine or N-bromosuccinimide in a suitable solvent like DCM or DMF at appropriate temperature to obtain compounds of Formula LVIII. 8-Aminoimidazo[1,5-a]pyrazine derivatives LIX can be prepared from compounds of Formula LVIII using ammonia(gas) in isopropanol at elevated temperature in a pressure vessel or microwave (>4 atm.) Compounds of Formula LX can be prepared from compound LIX using an appropriate boronic acid or pinacolester (V), in the presence of a suitable palladium catalyst system, for example bis(diphenylphosphine)palladium(0) palladium(II)chloride complex or tetrakis(triphenylphos-phine)palladium(0) in the presence of an inorganic base like potassium carbonate, cesium carbonate or potassium phosphate in a suitable solvent system like combinations of dioxane and water. Deprotection of derivatives of Formula LX can accomplished using methods known by those skilled in the art, such as TBAF of a strong acid like TFA. Subsequently, compounds of Formula LXI can be obtained with terminal alkenes containing an acid functionality, an appropriate coupling-reagent such as HATU of EDCI.HCl in a suitable solvent like DMF, THF or DCM at appropriate temperature. Ring closing metathesis towards compounds of Formula I-b can be accomplished with an appropriate Grubbs catalyst, molybdene or ruthenium catalyst in a suitable solvent like DCM or toluene at appropriate temperature. Subsequent removal of the DMB-group using methods known by skilled organic chemists, such as TFA containing triethylsilane provides isomeric mixtures of compounds of Formula I-b. The thus obtained mixtures of cis/trans isomers of Formula I-b could be separated using chromatographic techniques such as HPLC to obtain compounds of Formula I-b.

Alternatively compounds of Formula I-a to I-h, wherein R¹ to R⁴ and W, V and U have the previously defined meanings, can be prepared by the general synthetic route shown in scheme XI. Scheme XI shows a general synthetic route in relation to an exemplary compound of Formula I-a.

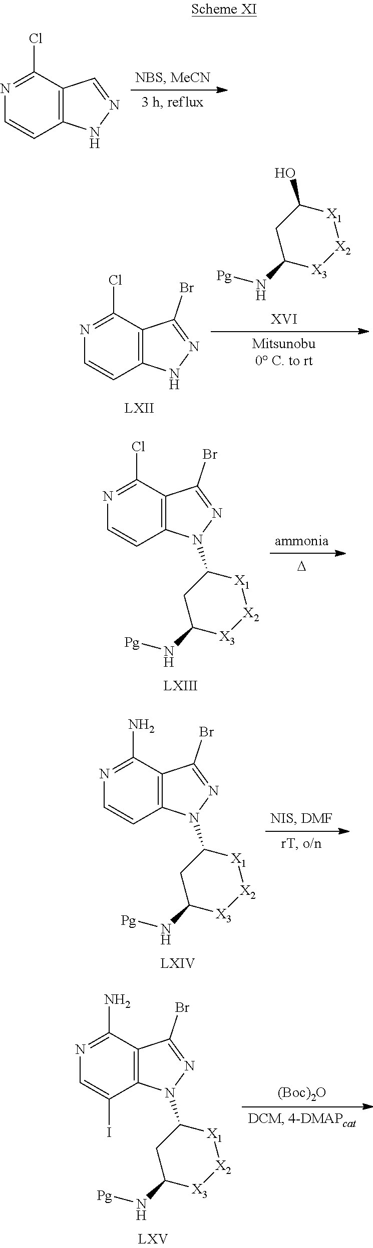

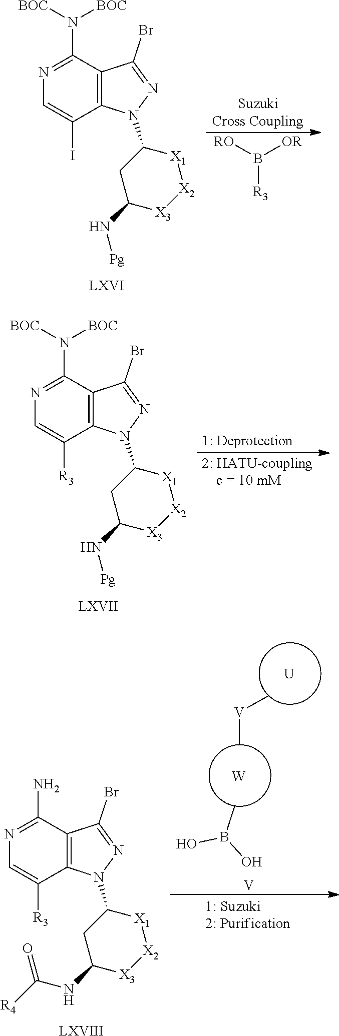

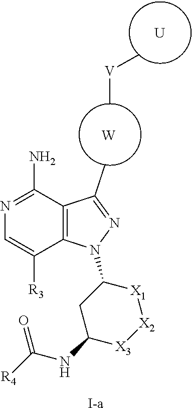

4-Chloro-3-bromo-1 H-pyrazolo[4,3-c]pyridine LXII can be prepared from commercially available 4-chloro-1 H-pyrazolo[4,3-c]pyridine using N-bromosuccinimide in a solvent such as acetonitrile or DMF at elevated temperatures. Compounds of Formula LXIII can, subsequently, be prepared from compound LXII and Compound XVI using Mitsunobu conditions, for example DIAD/triphenylphosphine in THE at 0° C. 1 H-pyrazolo[4,3-c]pyridin-4-amine derivatives LXIV can be prepared from compounds of Formula LXIII using ammonia(gas) in isopropanol or 25% aq. ammonia at elevated temperature in a pressure vessel or microwave (>4 atm.). Compounds of Formula LXV can be prepared from compound LXIV using N-iodosuccinimide in a solvent such as acetonitrile or DMF at room temperature. Subsequent amine protection of compound LXV using for example Boc₂O or Z-ONSu provide compounds of Formula LXVI. Compounds of Formula LXVII can be prepared from compound LXVI using an appropriate boronic acid or pinacolester, in the presence of a suitable palladium catalyst system, for example CataCXium® A Pd G3 or bis(diphenylphosphine)palladium(II)chloride complex in the presence of an inorganic base like potassium carbonate, cesium carbonate or potassium phosphate in a suitable solvent system like combinations of dioxane and water. Derivatives of Formula LXVIII can be prepared from derivatives of Formula LXVII after deprotection of the amino group with TBAF or a strong acid like TFA and subsequent carboxyl acid deprotection using a suitable inorganic base like lithium hydroxide or sodium hydroxide. Macrocyclization towards compounds of Formula LXVIII can be accomplished with an appropriate couplings reagent such as HATU of EDCI.HCl in a suitable solvent like DMF at appropriate temperature. Finally conversion of compounds of Formula LXVIII to compounds of Formula I-a can be accomplished using an appropriate boronic acid or pinacolester (V), in the presence of a suitable palladium catalyst system, for example bis(diphenylphosphine)palladium(0) palladium(II)chloride complex or tetrakis(triphenylphos-phine)palladium(0) in the presence of an inorganic base like potassium carbonate, cesium carbonate or potassium phosphate in a suitable solvent system like combinations of dioxane and water.

Alternatively compounds of Formula I-a to I-h, wherein R¹ to R⁴ and W, V and U have the previously defined meanings, can be prepared by the general synthetic route shown in scheme XII. Scheme XII shows a general synthetic route in relation to an exemplary compound of Formula 1-b.

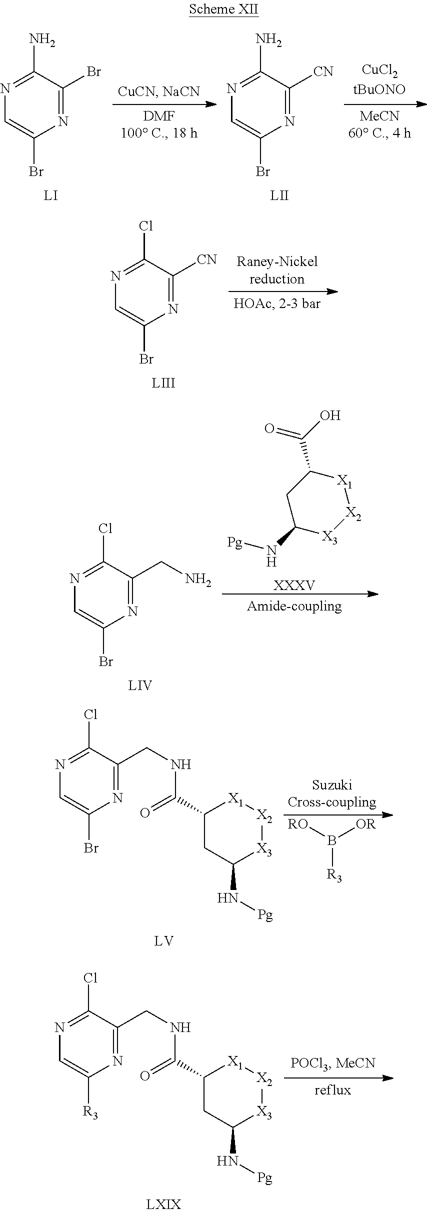

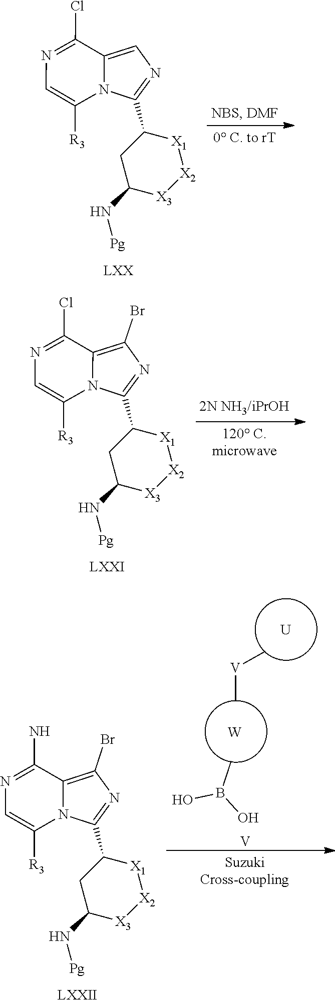

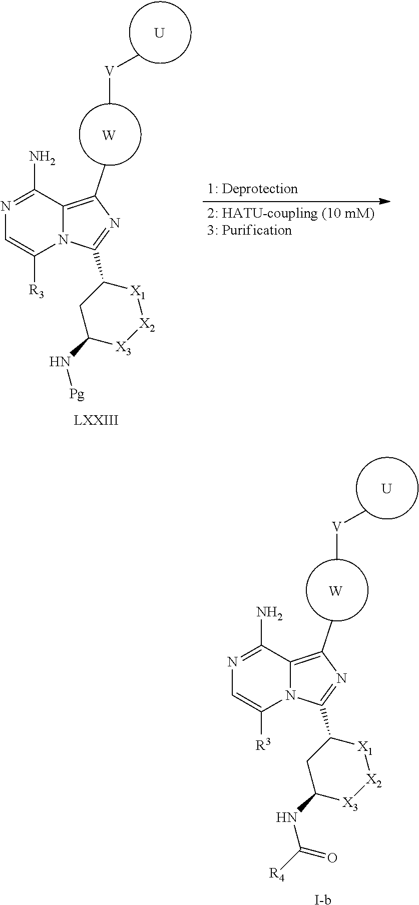

3-Amino-6-bromo-pyrazine-2-carbonitrile (LII) can be prepared from commercial available 2-amino-3,5-dibromo pyrazine (LI) using copper cyanide and sodium cyanide in DMF at elevated temperature. The resulting product can then be converted to 6-bromo-3-chloro-pyrazine-2-carbonitrile (LIII) under Sandmeyer conditions with copper chloride, tert-butylnitrite in an appropriate solvent such as acetonitrile under heating. Reduction of derivative LIII can be accomplished by hydrogenation under elevated pressure in the presence of a suitable catalyst system and solvent, for example Raney-Nickel to provide (6-bromo-3-chloro-pyrazin-2-yl)methanamine (LIV). This can then be reacted with an appropriately amine protected amino acid (XXXV) in a solvent such as DMF, THE or DCM in the presence of a base such as DIPEA, N-methylmorpholine, 4-DMAP or triethylamine and in the presence of a coupling reagent such as PyBOP, TBTU, EDCI or HATU to form compounds of Formula LV. Compounds of Formula LXIX can be prepared from compound LV using an appropriate boronic acid or pinacolester, in the presence of a suitable palladium catalyst system, for example CataCXium® A Pd G3 or bis(diphenylphosphine)palladium(0) palladium(II)chloride complex in the presence of an inorganic base like potassium carbonate, cesium carbonate or potassium phosphate in a suitable solvent system like combinations of dioxane and water. Cyclisation chloropyrazine of Formula LXIX can be performed using condensation reagents like phosphorus oxychloride under heating conditions to provide compounds of Formula LXX. Subsequent bromination can be accomplished using bromine or N-bromosuccinimide in a suitable solvent like DCM or DMF at appropriate temperature to obtain compounds of Formula LXXI. 8-Aminoimidazo[1,5-a]pyrazine derivatives LXXII can be prepared from compounds of Formula LXXI using ammonia(gas) in isopropanol at elevated temperature in a pressure vessel or microwave (>4 atm.) Compounds of Formula LXXIII can be prepared from compound LXXII using an appropriate boronic acid or pinacolester (V), in the presence of a suitable palladium catalyst system, for example bis(diphenylphosphine)palladium(0) palladium(II)chloride complex or tetrakis(triphenylphos-phine)palladium(0) in the presence of an inorganic base like potassium carbonate, cesium carbonate or potassium phosphate in a suitable solvent system like combinations of dioxane and water. Derivatives of Formula I can be prepared from derivatives of Formula LXXIII after deprotection of the amine group with TBAF or a strong acid like TFA and subsequent carboxyl acid deprotection using a suitable inorganic base like lithium hydroxide or sodium hydroxide. Following macrocyclization towards compounds of Formula 1-b can be accomplished with an appropriate coupling-reagent such as HATU of EDCI.HCl in a suitable solvent like DMF at appropriate temperature. After purification using chromatographic techniques such as HPLC compounds of Formula 1-b could be obtained.

The invention is illustrated by the following examples.

### Examples

The following examples are illustrative embodiments of the invention, not limiting the scope of the invention in any way. Reagents are either commercially available or are prepared according to procedures known in the literature.

### Method LCMS (A)

LC-MS system equipped with a Waters 2998 Photodiode Array Detector, Waters Acquity QDa Detector, Waters 2767 autosampler and Waters 2545 binary gradient module was used for sample analyses with a XTerra® MS C18 column (2.5 μm, 4.6×50 mm) for 10 min measurements.

The eluents used for this system are A (95/5 v/v % Milli-Q water/acetonitrile+0.1% formic acid) and B (acetonitrile+0.1% formic acid).

Method LCMS (A): 95% A to 95% B in 7 min, then 95% A.

### Method LCMS (B)

LC-MS system equipped with a Waters 2998 Photodiode Array Detector, Waters Acquity QDa Detector, Waters 2767 autosampler and Waters 2545 binary gradient module was used for sample analyses with a XBridge® MS C18 column (5 μm, 4.6×50 mm) for 30 min measurements.

The eluents used for this system are A (95/5 v/v % Milli-Q water/acetonitrile+0.1% formic acid) and B (acetonitrile+0.1% formic acid).

Method LCMS (B): 95% A to 95% B in 22 min, then switched to 95% A.

### Method Preparative HPLC

LC-MS system equipped with a Waters 2998 Photodiode Array Detector, Waters Acquity QDa Detector, Waters 2767 autosampler and Waters 2545 binary gradient module was used for Preparative reversed phase chromatography with a Luna© 5 μm C18(2) 100 Å (150×21 mm).

The eluents used for this system are A (95/5 v/v % Milli-Q water/acetonitrile+0.1% formic acid) and B (acetonitrile+0.1% formic acid).

The following abbreviations are used throughout the application with respect to chemical terminology:
TFA Trifluoracetic acid
HATU O-(7-Azabenzotriazol-1-yl)-1,1,3,3-tetramethyluroniumhexafluorophosphate
DMF N,N-Dimethylformamide
THE Tetrahydrofuran
DCM Dichloromethane
TMS-CI Chlorotrimethylsilane
DiPEA N,N-Diisopropylethylamine
HPLC High Performance Liquid Chromatography
LCMS Liquid Chromatography with Mass Spectrometry detection
4-DMAP 4-Dimethylamino pyridine
Boc tert-Butyloxycarbonyl
Cbz Benzyloxycarbonyl
LiHMDS Lithium bis(trimethylsilyl)amide
DBU 1,8-Diazabicyclo[5.4.0]undec-7-ene
DEAD Diethyl azodicarboxylate
o/n Overnight
Pd(dppf)Cl₂ 1,1′-bis(diphenylphosphino)ferrocene palladium(II) chloride
AIBN Azobisisobutyronitril
ZrCp₂(H)CI zirconocene hydrochloride (Schwartz Reagent)
¹H-NMR Proton nuclear magnetic resonance
BOC₂O Di-tert-butyl decarbonate
DPPA Diphenylphosphoryl azide
T3P Propylphosphonic anhydride
NIS N-Iodosuccinimide
PyBOP benzotriazol-1-yloxytripyrrolidinophosphonium hexafluorophosphate
TBTU 2-(1 H-Benzotriazole-1-yl)-1,1,3,3-tetramethylaminium tetrafluoroborate
EDCI 1-Ethyl-3-(3-dimethylaminopropyl)carbodiimide
2MeTHF 2-Methyltetrahydrofuran
Z-ONSu N-(Benzyloxycarbonyloxy)succinimide
o/w Over the weekend
TCEP Tris(2-carboxyethyl)phosphine
Tris 2-Amino-2-(hydroxymethyl)propane-1,3-diol

The names of the final products in the intermediates and examples are generated using Biovia Draw (version 16.1). In cases were Biovia Draw could not generate a name, molecular structures are given.

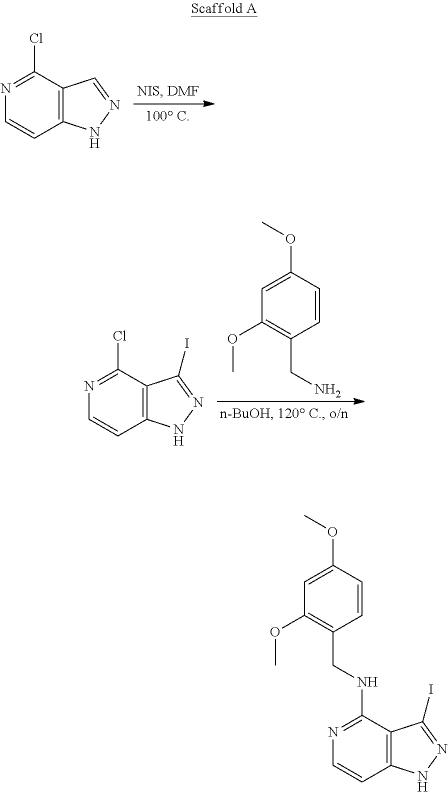

### N-[(2,4-dimethoxyphenyl)methyl]-3-iodo-1 H-pyrazolo[4,3-c]pyridin-4-amine (a) 4-Chloro-3-iodo-1 H-pyrazolo[4,3-c]pyridine

To a solution of 4-chloro-1 H-pyrazolo[4,3-c]pyridine (50 g, 325.6 mmol) in DMF (500 mL) was added N-iodosuccinimide (80.6 g, 353.1 mmol) and the mixture was stirred at 100° C. for 1 h. The mixture was cooled and added slowly to a mixture of 5% sodium thiosulfate/sodium bicarbonate solution/water (1 L/500 mL/500 mL). The mixture was transferred to a separation funnel and extracted with ethyl acetate (total of 3.5 L). The ethyl acetate layer was separated, washed with water (750 mL) and brine (500 mL), dried over sodium sulfate, filtered and concentrated under reduced pressure (still contains>25 mL of DMF). To the resulting suspension was added 100 mL of ethyl acetate and under stirring 250 mL of hexane. The solvent was decanted and the resulting suspension was again treated with ethyl acetate (50 mL) and hexane 250 mL. The precipitate was filtered and dried under vacuum to give 67.7 g of the title compound as a slightly brown powder (Yield: 74.4%).

### (b)N-[(2,4-dimethoxyphenyl)methyl]-3-iodo-1 H-pyrazolo[4,3-c]pyridin-4-amine (Scaffold A

To a suspension of 4-chloro-3-iodo-1 H-pyrazolo[4,3-c]pyridine (67.7 g, 242.2 mmol) in 1-butanol (675 mL) at room temperature was added 2,4-dimethoxybenzylamine (121.5 g, 726.6 mmol) and the mixture was heated at 120° C. and stirred overnight. After cooling to room temperature, the reaction solution was concentrated under reduced pressure. The precipitate was suspended in water and extracted with ethyl acetate. The ethyl acetate layers were washed with water, brine, dried over sodium sulfate, filtered and concentrated under reduced pressure to give the crude title compound. The crude was suspended/dissolved in dichloromethane (350 mL) and refluxed at 50° C. Hexane (350 mL) was added dropwise under reflux and stirred for 1 h. After cooling, the precipitate formed was filtered, washed with dichloromethane/hexane=1/1 v/v % and dried under vacuum at 40° C. to give 75.82 g of the title compound as an off-white powder (Yield: 76.3%).

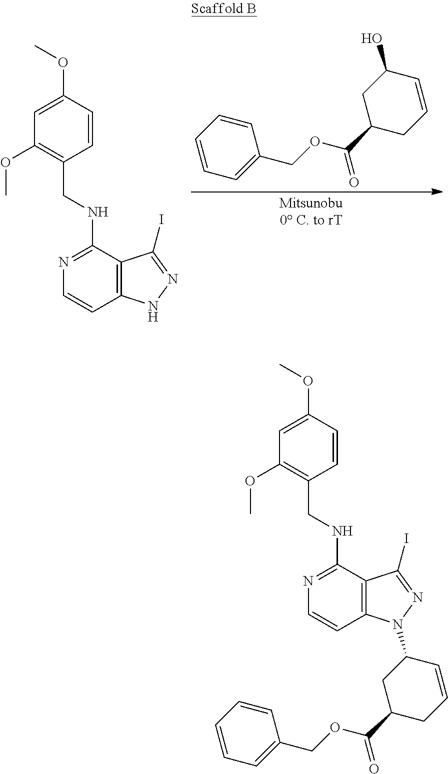 [SMILES: COc1ccc(CNc2nccc3[nH]nc(I)c23)c(OC)c1 ; O=C(OCc1ccccc1)[C@@H]1CC=C[C@H](O)C1 ; COc1ccc(CNc2nccc3c2c(I)nn3[C@@H]2C=CC[C@@H](C(=O)OCc3ccccc3)C2)c(OC)c1]

### Benzyl(1R,5S)-5-[4-[(2,4-dimethoxyphenyl)methylamino]-3-iodo-pyrazolo[4,3-c]pyridin-1-yl]cyclohex-3-ene-1-carboxylate (Scaffold B)

To an ice-cold (4° C.) suspension of N-[(2,4-dimethoxyphenyl)methyl]-3-iodo-1H-pyrazolo[4,3-c]pyridin-4-amine (Scaffold A) (41.02 g, 100 mmol), benzyl(1R,5R)-5-hydroxycyclohex-3-ene-1-carboxylate (Intermediate RP1) (25.54 g, 110 mmol) and triphenylphosphine (30.14 g, 115 mmol) in toluene (400 mL) was added dropwise a solution of diisopropyl azodicarboxylate (24.07 mL, 115 mmol) in toluene (100 mL). The mixture was stirred for 30 min at 4° C. and then stirred for 2 h at room temperature. The mixture was concentrated under reduced pressure. The residue was purified by column chromatography using SiO₂ and DCM/acetone=97/3 to 95/5 v/v %. All fractions containing the title compound were collected and concentrated in vacuo to give 50 g of benzyl(1R,5S)-5-[4-[(2,4-dimethoxyphenyl)methylamino]-3-iodo-pyrazolo[4,3-c]pyridin-1-yl]cyclohex-3-ene-1-carboxylate (Scaffold B (yield 80%).

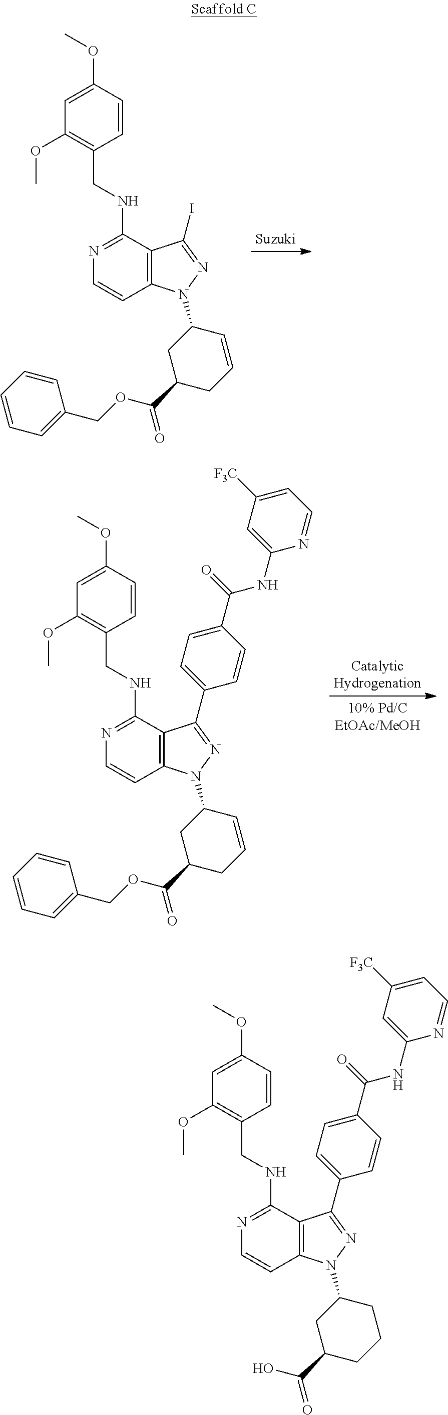 [SMILES: COc1ccc(CNc2nccc3c2c(I)nn3[C@@H]2C=CC[C@@H](C(=O)OCc3ccccc3)C2)c(OC)c1 ; COc1ccc(CNc2nccc3c2c(-c2ccc(C(=O)Nc4cc(C(F)(F)F)ccn4)cc2)nn3[C@@H]2C=CC[C@@H](C(=O)OCc3ccccc3)C2)c(OC)c1 ; COc1ccc(CNc2nccc3c2c(-c2ccc(C(=O)Nc4cc(C(F)(F)F)ccn4)cc2)nn3[C@@H]2CCC[C@@H](C(=O)O)C2)c(OC)c1]

### (1R,3R)-3-[4-[(2,4-Dimethoxyphenyl)methylamino]-3-[4-[[4-(trifluoromethyl)-2-pyridyl]carba-moyl]phenyl]ovrazolo[4.3-c]ovridin-1-yl]cyclohexanecarboxylic acid (Scaffold C)

### (a) benzyl(1R,5S)-5-[4-[(2,4-dimethoxyphenyl)methylamino]-3-[4-[[4-(trifluoromethyl)-2-pyridyl]carbamoyl]phenyl]pyrazolo[4,3-c]pyridin-1-yl]cyclohex-3-ene-1-carboxylate

Benzyl(1R,5S)-5-[4-[(2,4-dimethoxyphenyl)methylamino]-3-iodo-pyrazolo[4,3-c]pyridi-ne-1-yl]cyclohex-3-ene-1-carboxylate (15.88 g, 25.43 mmol) was dissolved in dioxane/water=4/1 v/v % (125 mL) and potassium carbonate (10.54 g, 76.29 mmol) was added. The solution was purged with nitrogen for 5 min and tert-4-(4,4,5,5-tetramethyl-1,3,2-dioxaborolan-2-yl)-N-[4-(trifluoromethyl)-2-pyridyl]benzamide (10.96 g, 27.97 mmol) and Pd(dppf)Cl₂.CH₂Cl₂ (1.03 g, 1.27 mmol) were added. The reaction mixture was stirred for 2 h at 80° C. The reaction mixture was diluted with ethyl acetate and filtered over Decalite™. The filtrate was concentrated under reduced pressure. The residue was purified by column chromatography using SiO₂ and ethyl acetate/heptane=¼ to 3/1 v/v %. All fractions containing the title compound were collected and concentrated in vacuo to give 18.5 g (Yield 95.0%).

### (b) (1R,3R)-3-[4-[(2,4-Dimethoxyphenyl)methylamino]-3-[4-[[4-(trifluoromethyl)-2-pyridyl]carba-moyl]phenyl]pyrazolo[4,3-c]pyridin-1-yl]cyclohexanecarboxylic acid (Scaffold C)

To a solution of benzyl(1R,5S)-5-[4-[(2,4-dimethoxyphenyl)methylamino]-3-[4-[[4-(trifluoromethyl)-2-pyridyl]carbamoyl]phenyl]pyrazolo[4,3-c]pyridin-1-yl]cyclohex-3-ene-1-carboxylate (18.0 g, 23.54 mmol) in ethyl acetate/methanol=4/1 v/v % (350 mL) was added 1.8 g of 10% Pd/C. Catalytic hydrogenation was performed for 16 h. at room temperature. The reaction was not completed. The benzylester was reduced completely but −31% of a double-bond containing product remainedt. The palladium-catalyst was filtered and the filtrate was recharged with 10% Pd/C (1.8 g) and catalytic hydrogenation was continued for 24 h. The palladium-catalyst was filtered and the filtrate was concentrated in vacuo to give 14.74 g of the title compound (Yield: 85.0%).

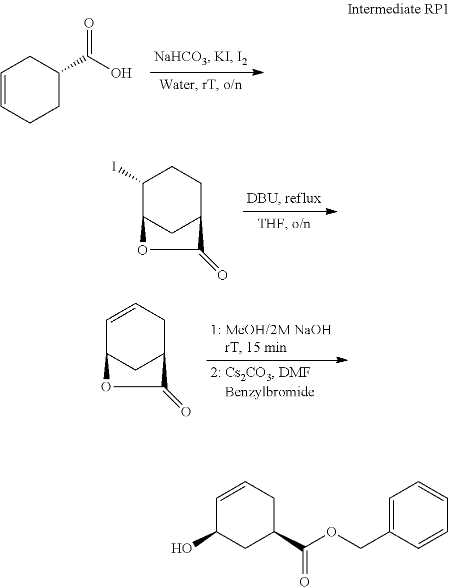 [SMILES: O=C(O)[C@H]1CC=CCC1 ; O=C1O[C@@H]2C[C@H]1CC[C@H]2I ; O=C1O[C@H]2C=CC[C@@H]1C2 ; O=C(OCc1ccccc1)[C@@H]1CC=C[C@H](O)C1]

### Benzyl(1R,5R)-5-hydroxycyclohex-3-ene-1-carboxylate

### (a) (1R,4R,5R)-4-iodo-6-oxabicyclo[3.2.1]loctan-7-one

(R)-(+)-3-Cyclohexenecarboxylic acid (50.7 g, 402 mmol) was suspended in H₂O (400 mL) under nitrogen. The mixture was cooled to 4° C. and sodium bicarbonate (101.3 g, 1.21 mol) was added, followed by a solution of potassium iodide (333 g, 2.01 mol) and iodine (107 g, 422 mmol) in H₂O (400 mL). The reaction was allowed to come to room temperature and stirred o/n and then extracted with dichloromethane (4×100 mL). The combined organic layers were washed with sat. NaHSO₃-solution (2×50 mL). The organic layer was protected from light, dried over Na₂SO₄, filtered and concentrated (20 mbar) to afford (1R,4R,5R)-4-iodo-6-oxabicyclo[3.2.1]octan-7-one (90.1 g, 89.0%) as an off-white solid.

### (b) (1R,5R)-6-oxabicyclo[3.2.1]loct-3-en-7-one

(1R,4R,5R)-4-iodo-6-oxabicyclo[3.2.1]octan-7-one (90.1 g, 358 mmol) was dissolved in dry THE (650 mL). Then, DBU (77 mL, 515 mmol) was added and the mixture was refluxed for 6 h. After cooling to room temperature, the suspension was filtered through Celite™, and concentrated in vacuo to −250 mL. This was used directly in the next step.

### (c) Benzyl(1R,5R)-5-hydroxycyclohex-3-ene-1-carboxylate (Intermediate RP1)

To the THE solution of (1R,5R)-6-oxabicyclo[3.2.1]oct-3-en-7-one (0.4 mol) in methanol (300 mL) was added 2M NaOH-solution (300 mL) and the mixture was stirred for 15 min at room temperature. The reaction was quenched by addition of 3M HCl-solution (300 mL) and the water layer was saturated by addition of sodium chloride. The mixture was extracted with ethyl acetate (3×100 mL). The combined organic phases were washed with brine, dried over sodium sulfate, filtered and the solvent removed in vacuo. The residue was dissolved in DMF (800 ml), cesium carbonate (129 g, 0.4 mol) and benzyl bromide (57 mL, 0.48 mol) were added subsequently. The mixture was stirred at room temperature for 30 min. The precipitate formed was filtered and the precipitate was washed with diethylether. The filtrate was washed with water, brine, dried over sodium sulfate and evaporated under reduced pressure. The residue was purified by column chromatography (heptane/ethyl acetate=95/5 to 45/55 v/v %) to give benzyl(1R,5R)-5-hydroxycyclohex-3-ene-1-carboxylate (57.1 g, 61.5% over 3 steps) as a cream-coloured oil.

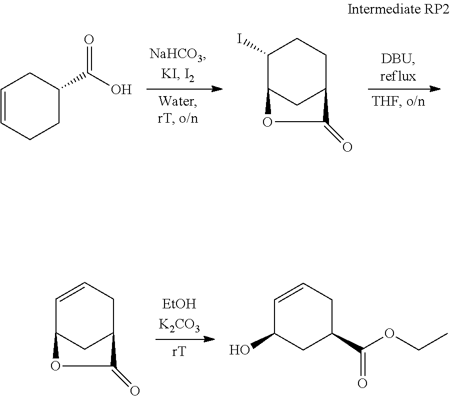

### Ethyl(1R,5R)-5-hydroxycyclohex-3-ene-1-carboxylate

### (a) (1R,4R,5R)-4-iodo-6-oxabicyclo[3.2.1]octan-7-one

(R)-(+)-3-Cyclohexenecarboxylic acid (50.7 g, 0.4 mol) was suspended in H₂O (400 mL) under nitrogen. The reaction mixture was cooled to 4° C. and sodium bicarbonate (101 g, 1.2 mol) was added, followed by a solution of potassium iodide (333 g, 2 mol) and iodine (107 g, 0.42 mol) in H₂O (400 mL). The reaction was allowed to come to room temperature and stirred o/n and then extracted with DCM (4×150 mL). The combined organic layers were washed with a solution of Na₂S203 (120 g) in H₂O (600 mL). The aqueous layer was extracted with DCM (2×150 mL). The combined organic layers were protected from light, dried over Na₂SO₄, filtered and concentrated (20 mbar) to afford (1R,4R,5R)-4-iodo-6-oxabicyclo[3.2.1]octan-7-one (95.22 g, 94.5%) as an off-white solid.

### (b) (1R,5R)-6-oxabicyclo[3.2.1]loct-3-en-7-one

(1R,4R,5R)-4-iodo-6-oxabicyclo[3.2.1]octan-7-one (95.22 g, 377.9 mmol) was dissolved in dry THE (700 mL). Then, DBU (86.3 g, 566.9 mmol) was added and the mixture was refluxed o/n. The reaction mixture was cooled to room temperature, diluted with diethylether (500 mL) and extracted with aq. HCl (1 L, 1 M) and brine (250 mL). The aqueous layers were extracted with diethylether (2×480 mL). The combined organic layers were dried over sodium sulfate, filtered and concentrated (350 mbar to afford (1R,5R)-6-oxabicyclo[3.2.1]oct-3-en-7-one quantitatively as a yellowish oil which was used directly in the next step.

### (c) Ethyl(1R,5R)-5-hydroxycyclohex-3-ene-1-carboxylate (Intermediate RP2)

To a stirred solution of (1R,5R)-6-oxabicyclo[3.2.1]oct-3-en-7-one (377.9 mmol, theor.) in ethanol (750 mL) was added potassium carbonate (10.45 g, 75.6 mmol) at room temperature and the mixture stirred o/n. The reaction mixture was filtered through a Celite pad. Removal of ethanol under reduced pressure afforded the crude product that was purified by column chromatography plug filtration (eluent 40% EtOAc/heptane) to afford the title compound (41.38 g, 60.8% over 3 steps and column) as a yellow liquid.

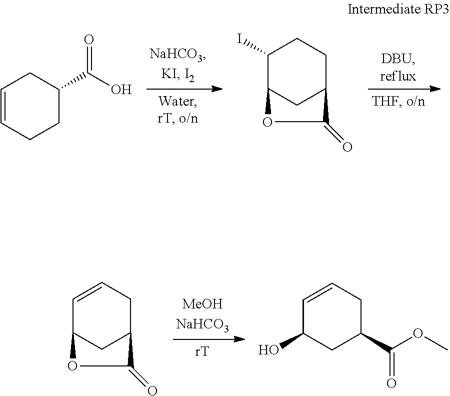

### Methyl(1R,5R)-5-hydroxycyclohex-3-ene-1-carboxylate

### (a) (1R,4R,5R)-4-iodo-6-oxabicyclo[3.2.1]octan-7-one

(R)-(+)-3-Cyclohexenecarboxylic acid (20.2 g, 160 mmol) was suspended in H₂O (430 mL) under nitrogen. The reaction mixture was cooled to 0° C. and sodium bicarbonate (40.3 g, 480.3 mmol) was added, followed by a solution of potassium iodide (159.5 g, 961 mmol) and iodine (39.6 g, 168 mmol) in H₂O (360 mL). The reaction was allowed to come to room temperature and stirred o/n and then extracted with DCM (3×150 mL). The combined organic layers were washed with a solution of Na₂S203 (120 g) in H₂O (600 mL). The aqueous layer was extracted with DCM (2×150 mL). The combined organic layers were protected from light, dried over Na₂SO₄, filtered and concentrated (20 mbar) to afford (1R,4R,5R)-4-iodo-6-oxabicyclo[3.2.1]octan-7-one (37.88 g, 93.9%) as an off-white solid.

### (b) (1R,5R)-6-oxabicyclo[3.2.1]loct-3-en-7-one

(1R,4R,5R)-4-iodo-6-oxabicyclo[3.2.1]octan-7-one (37.88 g, 150.3 mmol) was dissolved in dry THE (750 mL). Then, DBU (34.3 g, 225.2 mmol) was added and the mixture was refluxed o/n. The reaction mixture was cooled to room temperature, diluted with diethylether (480 mL) and extracted with aq. HCl (1 L, 0.5 M) and brine (1 L). The aqueous layers were extracted with diethylether (2×480 mL). The combined organic layers were dried over sodium sulfate, filtered and concentrated (350 mbar to afford (1R,5R)-6-oxabicyclo[3.2.1]oct-3-en-7-one quantitatively as a yellowish oil which was used directly in the next step.

### (c) Methyl(1R,5R)-5-hydroxycyclohex-3-ene-1-carboxylate (Intermediate RP3)

Sodium bicarbonate (37.88 g, 0.451 mol) was added to a solution (1R,5R)-6-oxabicyclo[3.2.1]oct-3-en-7-one (150.3 mmol theor.) in anhydrous MeOH (300 mL). After stirring for 1 week at room temperature the solvent was removed in vacuo (40° C./300 mbar). The residue was diluted with water (500 mL) and extracted with dichloromethane (3×250 mL). The combined extracts were washed with brine, dried over sodium sulfate, filtered and concentrated under reduced pressure to give the title compound (21.2 g, 90.3%) as a slightly coloured liquid.

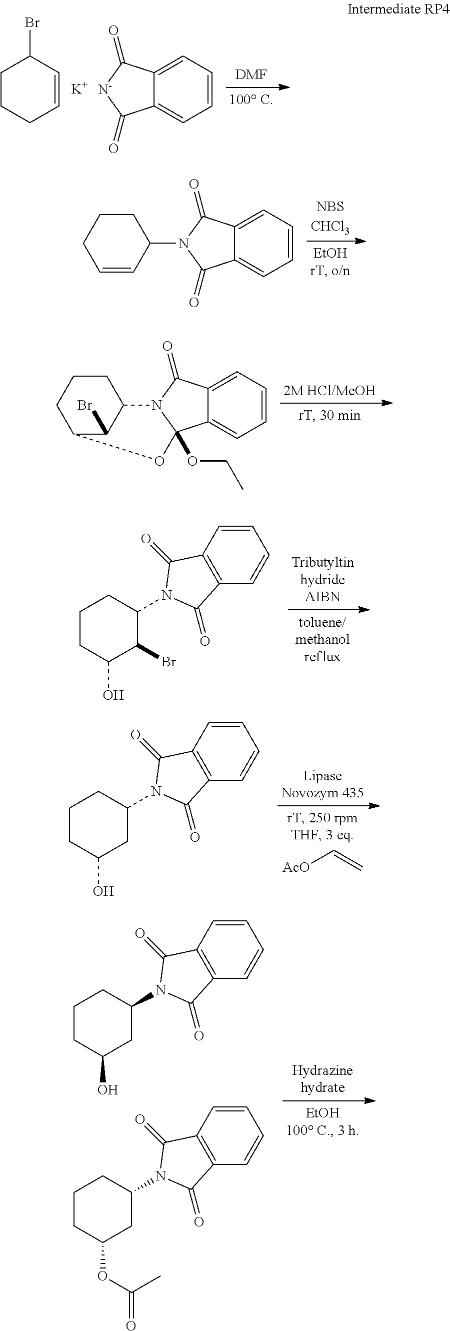

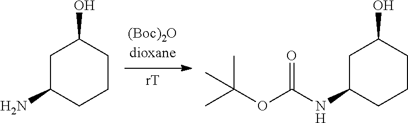

### tert-Butyl N-[(1R,3S)-3-hydroxycyclohexyl]carbamate

### (a) 2-Cyclohex-2-en-1-ylisoindoline-1,3-dione

A mixture of potassium phthalimide (69.88 g, 377.2 mmol) and 3-bromocyclohexene (60.75 g, 377.2 mmol) in DMF (500 mL) was stirred at 30° C. and gradually warmed to 100° C. for 6 h and then o/n by room temperature. The reaction mixture was diluted with water (2 L), extracted with ethyl acetate (3×500 mL). The combined organic layers were washed with water (2×500 mL), brine, dried over sodium sulfate, filtered and concentrated under reduced pressure.

The residue was purified by flash column chromatography (dichloromethane) to afford 63.29 g of the title compound (Yield: 73.8%).

### (b) (±) 13-Bromo-12b-ethoxy-2,6-methano[1,3]oxazocino[2,3a]iso-indol-8-one

N-Bromosuccinimide (61.95 g, 348 mmol) was added to a stirred solution of 2-cyclohex-2-en-1-ylisoindoline-1,3-dione (63.29 g, 278.4 mmol) in chloroform (1000 mL) and ethanol (65 mL) and the mixture was stirred at room temperature o/n. The mixture was washed with 1M sodium thiosulphate solution (1.5 L). The organic layer was separated and dried over sodium sulphate, filtered and concentrated under reduced pressure. The residue was purified by column chromatography (Heptane/Ethyl acetate=7/3 v/v %) to afford the title compound (97.0 g, 98.9%).

### (c) 1,2-Trans-2,3-trans-2-Bromo-3-N-phtalimidocyclohexanol

2M HCl-solution (150 mL) was added to a stirred solution of the orthoamide (86.44 g, 245.4 mmol) in methanol (600 mL) and the solution was stirred at room temperature for 30 min. Most of the solvent was removed and dichloromethane (300 mL) was added to the residue. This solution was then washed with water (2×50 mL), dried over sodium sulfate, filtered and concentrated under reduced pressure. The crystalline residue was recrystallised from ethyl acetate/hexane (200/500 mL @75° C.) to give the title compound (70.04 g, 88.0%)

### (d) cis-3-Phthalimidocyclohexanol

Tri-n-butyltin hydride (84 g, 288.7 mmol) was added to a stirred solution of 1,2-trans-2,3-trans-2-bromo-3-N-phtalimidocyclohexanol (78 g, 240.6 mmol) and AIBN (0.2M in toluene, 60 mL, 12 mmol) in toluene (700 mL) and methanol (70 mL) and the mixture was stirred at reflux o/n. Additional AIBN (2×5 mL) and tri-n-butyltin hydride (2×10 mL) were added and the reaction mixture was stirred at reflux o/n. Reaction proceeded slowly and additional AIBN (20 mL) and tri-n-butyltin hydride were added and the reaction mixture was stirred at reflux for 3h. Progress was followed by TLC. The mixture was then placed under nitrogen and additional AIBN (20 mL) and tri-butyltin hydride (20 g) were added subsequently. and stirred at reflux for 4 h. The reaction mixture was concentrated under reduced pressure and the residue was triturated with 750 mL heptane (2×). The heptane layer was removed by suction under reduced pressure. Again 100 mL ethyl acetate and 650 mL heptane were added. The precipitate was filtered, washed with hexane and dried under vacuum at 40° C. o/n to give 49.07 g of the title compound (yield: 83.1%).

### (e) 2-[(1R,3S)-3-hydroxycyclohexyl]isoindoline-1,3-dione+[(1R,3S)-3-(1,3-dioxoisoindolin-2-yl)cyclohexyl]acetate

To a solution of cis-3-phthalimidocyclohexanol (49 g, 200 mmol) in THE (600 mL) was added Lipase Novozyme 435 (25 g) and vinyl acetate (55.3 mL, 600 mmol). The resulting mixture was shaken at room temperature and 250 rpm o/n. The progress of the reaction was monitored by LC-MS. After o/n reaction, the enzyme was filtered off, washed with THE and the solvent was evaporated under reduced pressure. The residue was purified by column chromatography using SiO₂ and ethyl acetate/heptane=1/1 to 10/0 and to ethyl acetate/dichloromethane=3/1 v/v %. All fractions containing the title compound were collected and concentrated in vacuo to give 21.21 g of 2-[(1R,3S)-3-hydroxycyclohexyl]isoindoline-1,3-dione (yield 43.3%) and 31 g of [(1R,3S)-3-(1,3-dioxoisoindolin-2-yl)cyclohexyl]acetate (yield 54.0%).

### (f) (1 S.3R)-3-aminocyclohexanol

To a solution of 2-[(1R,3S)-3-hydroxycyclohexyl]isoidoline-1,3-dione (21.2 g, 86.4 mmol) in ethanol (280 mL) was added hydrazine hydrate (4.28 mL) and the reaction mixture was stirred at 100° C. o/n. The mixture was cooled to room temperature and the precipitate was filtered, washed with ethanol and the filtrate was concentrated under reduced pressure to give 9.87 g of the title compound as a yellow solid (yield: 59.9%). The crude precipitate 13.74 g still contains a lot of 2,3-dihydrophthalazine-1,4-dione and product (yield: 39.4%).

### (g) tert-Butyl N-[(1R,3S)-3-hydroxycyclohexyl]carbamate (Intermediate RP4)

To a solution of (1 S,3R)-3-aminocyclohexanol (9.87 g, 51.74 mmol) in dioxane (200 mL) was added di-tert-butyl dicarbonate (11.86 g) and the reaction mixture was stirred at room temperature o/n. Dioxane was partly evaporated and ethyl acetate (500 mL) was added to the suspension. The suspension was washed with NaOH-solution (4 g in 200 mL), water and brine, dried over sodium sulfate, filtered and concentrated under reduced pressure to give 12.5 g of the title compound.

Second batch: To a solution of (1S,3R)-3-aminocyclohexanol (13.74 g, 34.0 mmol) in dioxane (200 mL) was added di-tert-butyl dicarbonate (7.8 g) and the reaction mixture was stirred at room temperature o/w. Dioxane was partly evaporated and ethyl acetate (500 mL) was added to the suspension. The suspension was washed with NaOH-solution (4 g in 200 mL), water and brine, dried over sodium sulfate, filtered and concentrated under reduced pressure to give 7.54 g of the title compound.

Both batches were combined and suspended in ethyl acetate. Hexane was added and the precipitate formed was filtered, washed with hexane and dried under high vacuum at 40° C. to give 16.3 g of tert-butyl N-[(1R,3S)-3-hydroxycyclohexyl]carbamate (Intermediate RP4) (yield: 87.6%).

### (h) [(1 S,3R)-3-(tert-butoxycarbonylamino)cyclohexyl](2S)-3,3,3-trifluoro-2-methoxy-2-phenyl-propanoate

To a solution of tert-butyl N-[(1R,3S)-3-hydroxycyclohexyl]carbamate (50 mg, 0.23 mmol) in dichloromethane (2 mL) was added triethylamine (35.6 μL, 0.26 mmol) and 4-dimethylaminopyridine (3 mg, 0.023 mmol) and the mixture was stirred for 5 min. (R)-(−)-a-methoxy-a-trifluoromethylphenylacetyl chloride (61.6 mg, 0.24 mmol) was added and the reaction mixture was stirred at room temperature o/n. Dichloromethane was distilled off under reduced pressure, the resulting residue was purified by column chromatography (heptane/ethyl acetate=8/2 v/v %) to give 71.4 mg of the title compound (Yield: 72.0%). Both proton and fluor NMR showed that tert-butyl N-[(1R,3S)-3-hydroxycyclohexyl]carbamate (Intermediate RP4) has and d.e.>99.0%.

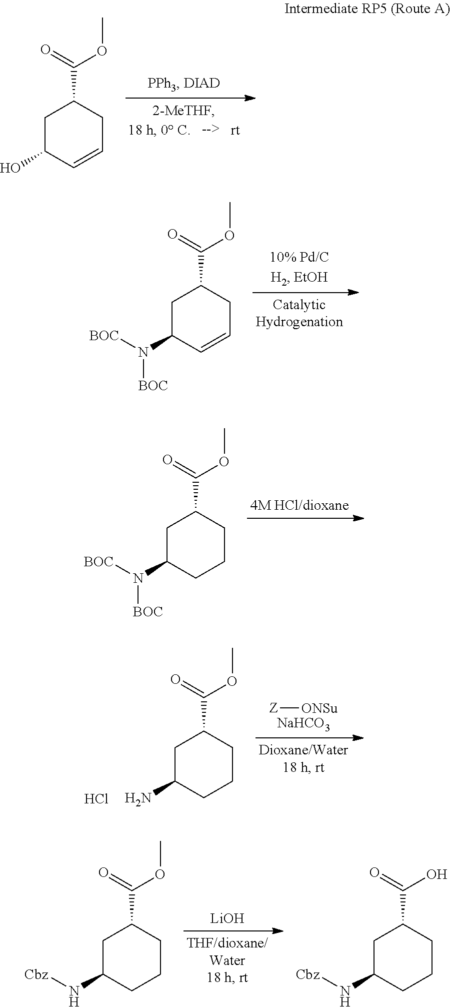

### (1R,3R)-3-(Benzyloxycarbonylamino)cyclohexanecarboxylic acid

### (a) Methyl (1R,5S)-5-[bis(tert-butoxycarbonyl)aminolcyclohex-3-ene-1-carboxylate

To an ice-cold (4° C.) solution of di-tert-butyl iminodicarboxylate (4.6 g, 21.2 mmol), methyl(1R,5R)-5-hydroxycyclohex-3-ene-1-carboxylate (Intermediate RP3) (3.31 g, 21.2 mmol) and triphenylphosphine (6.67 g, 25.4 mmol) in 2-MeTHF (180 mL) was added dropwise a solution of diisopropyl azodicarboxylate (6.26 mL, 31.8 mmol) in 2-MeTHF (30 mL). The mixture was stirred for 30 min at 4° C. and then allowed to warm to room temperature and stirred for 3 h. The mixture was concentrated under reduced pressure. The residue was purified by column chromatography using SiO₂ and heptane/ethyl acetate=3/1 v/v %. All fractions containing the title compound were collected and concentrated in vacuo to give 5.68 g of methyl(1R,5S)-5-[bis(tert-butoxycarbonyl)amino]cyclohex-3-ene-1-carboxylate (yield 75.4%).

### (b) Methyl(1R,3R)-3-[bis(tert-butoxycarbonyl)aminolcyclohexanecarboxylate

To a solution of methyl(1R,5S)-5-[bis(tert-butoxycarbonyl)amino]cyclohex-3-ene-1-carboxylate (2.48 g, 6.98 mmol) in ethanol (140 mL) was added 248 mg of 10% Pd/C. Catalytic hydrogenation was performed at room temperature for 16 h. The palladium-catalyst was filtered and the filtrate was concentrated in vacuo to give 2.6 g of the title compound (Yield: quantitative).

### (c) Methyl(1R,3R)-3-aminocyclohexanecarboxylate hydrochloride

To methyl(1R,3R)-3-[bis(tert-butoxycarbonyl)amino]cyclohexanecarboxylate (2.6 g, 7.2 mmol) was added 4M HCl/dioxane solution (18 mL) and the mixture was stirred at room temperature o/n. The mixture was concentrated in vacuo to give 1.06 g the title compound (yield: 76%).

### (d) Methyl(1R,3R)-3-(benzyloxycarbonylamino)cyclohexanecarboxylate

Methyl(1R,3R)-3-aminocyclohexanecarboxylate hydrochloride (1.06 g, 5.47 mmol) was suspended in 10 mL water. Sodium bicarbonate (1.38 g, 16.4 mmol) in 10 mL water was added followed by a drop-wise addition of a solution N-(benzyloxycarbonyloxy)succinimide (1.50 g, 6.01 mmol) in dioxane (30 mL). The reaction mixture was stirred at room temperature o/n. The mixture was diluted with ethyl acetate (50 mL) and water (50 mL) and the bi-phasic system was stirred 30 minutes at room temperature. The layers were separated and the water layer was extracted with ethyl acetate (2×20 mL). The combined organic layers were washed with water (50 mL), 0.5N aq. HCl-solution (50 mL), water (50 mL), 5% aq. NaHCO₃-solution (50 mL), water (50 mL) and brine (25 mL), dried (Na₂SO₄), filtered and concentrated in vacuo to give 1.78 g of the title compound (yield: quantitative, crude).

### (e) (1R,3R)-3-(Benzyloxycarbonylamino)cyclohexanecarboxylic acid (Intermediate RP5)

The crude product methyl(1R,3R)-3-(benzyloxycarbonylamino)cyclohexanecarboxy-late (1.45 g, 4.98 mmol) was dissolved in THF/dioxane/water=4/1/1 v/v % (74 mL) and subsequently lithium hydroxide (358 mg, 14.9 mmol) was added. The mixture was stirred at room temperature o/n. Ethyl acetate (50 mL) and water (were added) and the pH of the mixture was adjusted to pH<3 by addition of 2M HCl-solution. The organic phase was separated, washed with water, brine, dried over sodium sulfate, filtered and concentrated under reduced pressure to give 800 mg of (1R,3R)-3-(benzyloxycarbonylamino)cyclohexanecarboxylic acid (Intermediate RP5) (yield:57.9%).

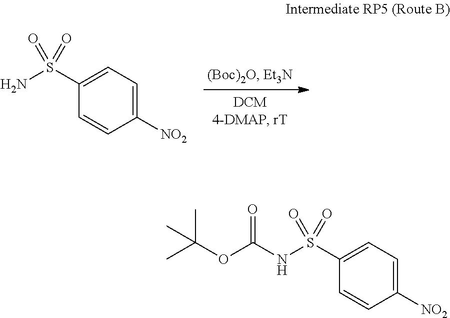 [SMILES: NS(=O)(=O)c1ccc([N+](=O)[O-])cc1 ; CC(C)(C)OC(=O)NS(=O)(=O)c1ccc([N+](=O)[O-])cc1]

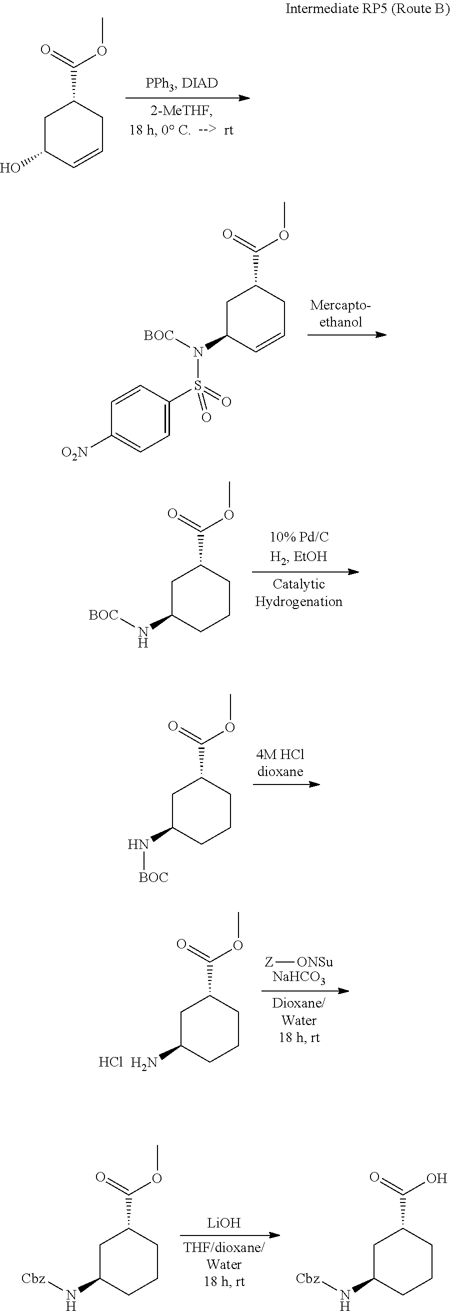

### (1R,3R)-3-(Benzyloxycarbonylamino)cyclohexanecarboxylic acid

### (a) tert-Butyl N-(4-nitrophenyl)sulfonylcarbamate

Triethylamine (10.4 mL, 74.62 mmol), 4-dimethylaminopyridine (605 mg, 4.95 mmol) and di-tert-butyl dicarbonate (13.5 g, 61.86 mmol) were added sequentially to a solution of 4-nitrobenzene sulfonamide (10 g, 49.46 mmol) in dichloromethane (100 mL). The reaction mixture was stirred for 30 minutes at room temperature. To the reaction mixture was added hydrochloric acid (1 N aqueous solution) until it becomes acidic. The organic layer was separated and washed with saturated sodium chloride aqueous solution, dried over sodium sulfate, filtered and then concentrated under reduced pressure. The residue was dissolved in dichloromethane and purified by column chormatography over silica (heptane to ethyl acetate=10/0 to 0/10) to give 13.09 g of the title compound (yield: 87.5%).

### (b) Methyl(1R,5S)-5-[tert-butoxycarbonyl-(4-nitrophenyl)sulfonyl-amino]-cyclohex-3-ene-1-carboxylate

To a cold (−20° C.) solution of methyl(1R,5R)-5-hydroxycyclohex-3-ene-1-carboxylate (15 g, 96.0 mmol), tert-butyl N-(4-nitrophenyl)sulfonylcarbamate (29.0 g, 96.0 mmol) and triphenylphosphine (27.7 g, 105.6 mmol) in THE (300 mL) was added dropwise a solution of diisopropyl azodicarboxylate (20.8 mL, 105.6 mmol) in THE (100 mL). The reaction mixture was concentrated under reduced pressure to give a residue which was purified by column chromatography (hexane/ethyl acetate=85/15 v/v %) to give 36 g of the title compound. (yield: 85.1%).

### (c) Methyl (1R,5S)-5-(tert-butoxycarbonylamino)cyclohex-3-ene-1-carboxylate

To a stirred solution of methyl(1R,5S)-5-[tert-butoxycarbonyl-(4-nitrophenyl)sulfonyl-amino]-cyclohex-3-ene-1-carboxylate (35.15 g, 79.8 mmol) in acetone (300 mL) was added DBU (23.85 mL, 159.6 mmol) and 2-mercaptoethanol (11.23 mL, 159.6 mmol). The reaction mixture was stirred for 3 h at room temperature. Acetone was removed under reduced pressure and the resulting residue was purified by column chromatography (hexane/ethyl acetate=90/10 to 88/12 v/v %) to give 14.1 g of the title compound (Yield: 69.2%) as a crystalline white solid.

### (d) Methyl(1R,3R)-3-(tert-butoxycarbonylamino)cyclohexanecarboxylate

To a solution of methyl(1R,5S)-5-(tert-butoxycarbonylamino)cyclohex-3-ene-1-carboxylate (14.9 g, 58.36 mmol) in methanol (300 mL) was added 1.5 g of 10% Pd/C. Catalytic hydrogenation was performed for 3 h at room temperature. The palladium-catalyst was filtered and the filtrate was concentrated in vacuo to afford the title compound in quantitative crude yield.

### (e) Methyl(1R,3R)-3-aminocyclohexanecarboxylate hydrochloride

To a cooled (4° C.) solution of methyl(1R,3R)-3-(tert-butoxycarbonylamino)cyclo-hexanecarboxylate (15.2 g, 58.36 mmol) in methanol (300 mL) was added drop-wise acetyl chloride (42 mL, 583.6 mmol). The reaction mixture was stirred for 1 h. The mixture was concentrated under reduced pressure and dried in vacuo to give the title compound in quantitative crude yield.

### (f) Methyl(1R,3R)-3-(benzyloxycarbonylamino)cyclohexanecarboxylate

To a cooled (4° C.) solution of methyl(1R,3R)-3-aminocyclohexanecarboxylate hydrochloride (58.36 mmol) in dioxane/water=1/1 v/v % (200 mL) was added portion-wise sodium bicarbonate (14.7 g, 175 mmol). To the resulting suspension was added drop-wise a solution of N-(benzyloxycarbonyloxy)succinimide (14.8 g, 59.02 mmol) in dioxane (150 mL) and the resulting mixture was stirred at room temperature o/w. LC-MS showed some starting material present. Additionally, 1.5 g of Z-ONSu was added as a solution in dioxane. The mixture was stirred at room temperature o/n. Ethyl acetate was added and the resulting mixture was washed with 0.5M HCl solution, water and brine. The organic layer was separated, dried over sodium sulfate, filtered and concentrated under reduced pressure and dried in vacuo to give the title compound in quantitative crude yield.

### (g) (1R,3R)-3-(benzyloxycarbonylamino)cyclohexanecarboxylic acid (Intermediate RP5)

To a solution of methyl(1R,3R)-3-(benzyloxycarbonylamino)cyclohexanecarboxylate (58.36 mmol) in THE/water=4/1 v/v % (375 mL) was added lithium hydroxide (4.21 g, 175 mmol) and the reaction mixture was stirred at room temperature o/n. Ethyl acetate (300 mL) and water (300 mL) were added to the mixture and the aqueous phase was separated. The ethyl acetate layer washed extracted with water (100 mL). The combined aqueous phases were washed with dichloromethane (100 mL) and acidified (pH<2) by addition of 2M HCl-solution (90 mL). The water layer was extracted with ethyl acetate (3×250 mL). The combined ethyl acetate layers were washed with water, brine, dried over sodium sulfate, filtered and concentrated under reduced pressure and dried in vacuo to give 15.72 g of the title compound (Yield: 96.7% over 4 steps).

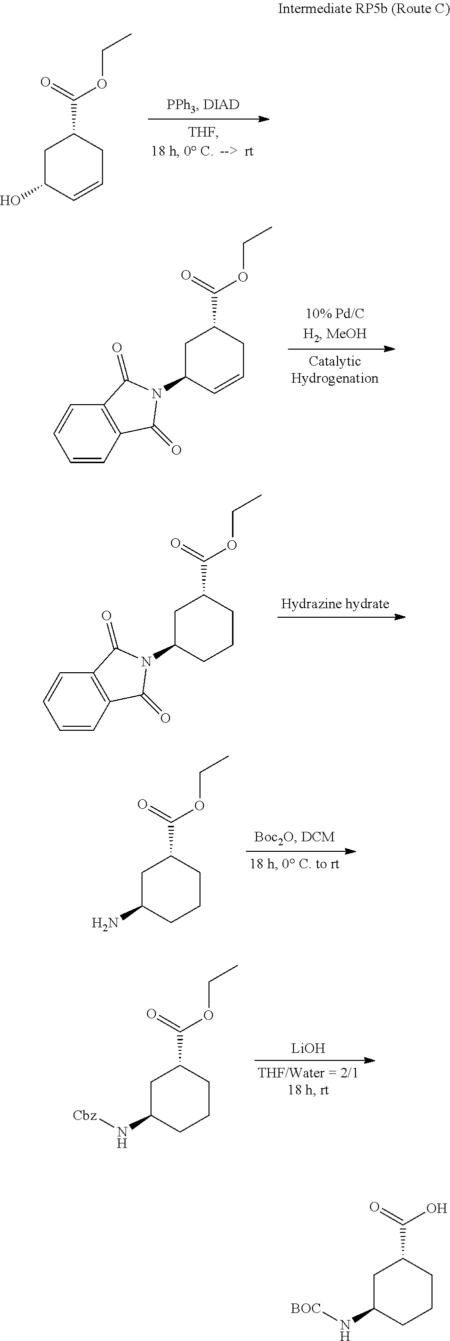

### (1R,3R)-3-(tert-butoxycarbonylamino)cyclohexanecarboxylic acid

### (a) Ethyl(1R,5S)-5-(1,3-dioxoisoindolin-2-yl)cyclohex-3-ene-1-carboxylate

To an ice-cold (0° C.) solution of ethyl(1R,5R)-5-hydroxycyclohex-3-ene-1-carboxylate (Intermediate RP2, 15.0 g, 88.13 mmol), phthalimide (14.26 g, 96.94 mmol) and triphenylphos-phine (34.67 g, 132.2 mmol) in toluene (264 mL) was added dropwise diisopropyl azodicarbo-xylate (26.02 mL, 132.2 mmol) in 10 min. The reaction mixture was stirred at 0° C. for 30 min and then allowed to come to room temperature and stirred for 3 h. The mixture was evaporated under reduced pressure to give a yellow oil. Heptane/ethyl acetate=7/3 v/v % (500 mL) was added and the mixture was heated to 70° C. After cooling, the mixture was stirred for 72 h at room temperature. The solids were filtered, washed with heptane/ethyl acetate=9/1 v/v % (2×50 mL) and the filtrate was evaporated under reduced pressure. The resulting residue was purified by column chromatography (heptane/ethyl acetate=9/1 to 6/4 v/v %) to give 21.96 g of the title compound (Yield: 83.0%) as an off-white solid.

### (b) Ethyl(1R,3R)-3-(1,3-dioxoisoindolin-2-yl)cyclohexanecarboxylate

To a solution of ethyl(1R,5S)-5-(1,3-dioxoisoindolin-2-yl)cyclohex-3-ene-1-carboxylate (27.73 g, 97.19 mmol) in methanol (975 mL) was added 2.7 g of 10% Pd/C. Catalytic hydrogenation was performed for 3 h at room temperature. The palladium-catalyst was filtered and the filtrate was concentrated in vacuo to afford 27.52 g the title compound (Yield: 94%).

### (c) Ethyl(1R,3R)-3-aminocyclohexanecarboxylate

To a solution of ethyl(1R,3R)-3-(1,3-dioxoisoindolin-2-yl)cyclohexanecarboxylate (26.5 g, 87.95 mmol) in ethanol (440 mL) was added drop-wise hydrazine hydrate (64% in water, 4.68 mL, 96.74 mmol). The reaction mixture was stirred for 30 min. at room temperature and then refluxed for 3 h. Additional hydrazine hydrate (425 μL) was added and stirring under reflux was continued for 2 h. The mixture was concentrated under reduced pressure and dried in vacuo to give the title compound in quantitative crude yield.

### (d) Ethyl (1R,3R)-3-(tert-butoxycarbonylamino)cyclohexanecarboxylate

To a cold (0° C.) stirred suspension of ethyl(1R,3R)-3-aminocyclohexanecarboxylate (91.27 mmol, theoretical) in dichloromethane (456 ml) was added portion-wise di-tert-butyl dicarbonate (21.91 g, 100.4 mmol). The reaction mixture was stirred for 15 min at 0° C., then allowed to come to room temperature. The mixture was washed with cold 0.5N NaOH-solution, water and brine. The organic layer was separated, dried over sodium sulfate, filtered and concentrated under reduced pressure. The resulting residue was purified by column chromatography (heptane/ethyl acetate=8/2 to 6/4 v/v %) to give 20.5 g of the title compound (Yield: 83.0% over two steps) as an off-white solid.

### (e) (1R,3R)-3-(tert-butoxycarbonylamino)cyclohexanecarboxylic acid

To a solution of ethyl(1R,3R)-3-(tert-butoxycarbonylamino)cyclohexanecarboxylate (20.5 g, 75.56 mmol) in THE (300 mL) was added a solution of lithium hydroxide (1.8 g, 75.56 mmol) in water (150 mL) and the reaction mixture was stirred at room temperature o/n. Additional lithium hydroxide (0.9 g) was added and stirring was continued for 24 h at room temperature. The mixture was acidified (pH<2) by addition of 1 M HCl-solution (131 mL). The water layer was separated and extracted with dichloromethane (2×100 mL). The combined organic layers were washed with water, brine, dried over sodium sulfate, filtered and concentrated under reduced pressure and dried in vacuo to give 16.81 g (91%) of (1R,3R)-3-(tert-butoxycarbonylamino)cyclo-hexanecarboxylic acid (Intermediate RP5b).

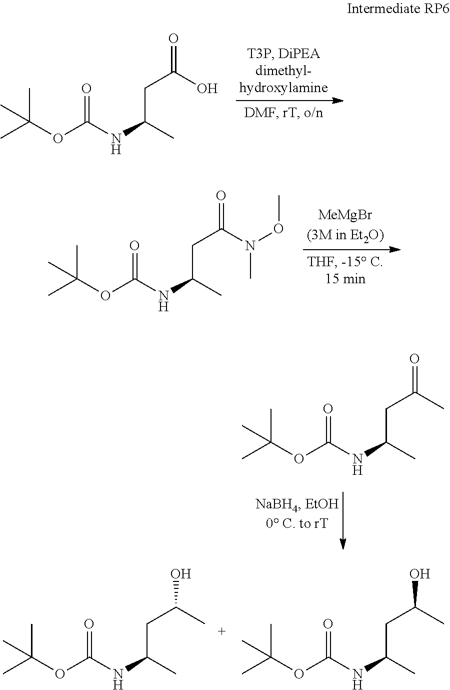

### tert-Butyl N-[(1R,3S)-3-hydroxy-1-methyl-butyl]carbamate

### (a) tert-Butyl N-[(1R)-3-[methoxy(methyl)amino]-1-methyl-3-oxo-propyl]carbamate

DiPEA (22.3 mL, 132 mmol) followed by T3P (50 wt % in EtOAc, 34 mL, 57 mmol) and N,O-dimethylhydroxylamine (6.43 g, 66 mmol) were added sequentially to a solution of (R)—N-Boc-3-aminobutyric acid (8.94 g, 44 mmol) in DMF (90 mL). The reaction mixture was stirred at room temperature o/n. Water (50 mL) was added and the aqueous mixture was stirred for 1 h. and then extracted with ethyl acetate. The organic extracts were combined, washed with 5% aq. NaHCO₃-solution, water, brine, dried over sodium sulfate, filtered and concentrated in vacuo to give 10.84 g (100%) of the title compound.

### (b) tert-Butyl N-[(1R)-1-methyl-3-oxo-butyl]carbamate

Methylmagnesium bromide (3 M in Et₂O, 32.3 mL, 96.8 mmol) was added dropwise to a solution of tert-butyl N-[(1R)-3-[methoxy(methyl)amino]-1-methyl-3-oxo-propyl]carbamate (10.84 g, 44 mmol) in THE (132 mL) at −15° C. under nitrogen. After stirring at this temperature for 15 min, the mixture was allowed to come to room temperature and stirring was continued for 1 h. The mixture was cooled to 0° C. and sat. aq. NH₄Cl-solution (40 mL) was added carefully. The aqueous mixture was extracted with ethyl acetate. The organic extracts were combined, washed with water, brine, dried over sodium sulfate, filtered and concentrated in vacuo. The residue was purified by column chromatography using SiO₂ and heptane/ethyl acetate=1/1 v/v %. All fractions containing the title compound were collected and concentrated in vacuo to give 3.3 g of tert-butyl N-[(1R)-1-methyl-3-oxo-butyl]carbamate (yield 37%).

### (c) tert-Butyl N-[(1R,3S)-3-hydroxy-1-methyl-butyl]carbamate (Intermediate RP6)

Sodium borohydride (744 mg, 19.68 mmol) was added to a cold (0° C.) solution of tert-butyl N-[(1R)-1-methyl-3-oxo-butyl]carbamate (3.3 g, 16.4 mmol) in ethanol (82 mL) under nitrogen. After stirring at this temperature for 5 min, the mixture was allowed to come to room temperature and stirring was continued for 1 h. The mixture was cooled to 0° C. and sat. aq. NH₄Cl-solution was added carefully. The aqueous mixture was extracted with ethyl acetate. The organic extracts were combined, washed with water, brine, dried over sodium sulfate, filtered and concentrated in vacuo. The residue was purified by column chromatography using SiO₂ and dichloromethane/TBME=10/0 to 6/4 v/v %. All fractions containing the title compound were collected and concentrated in vacuo to give 1.93 g (50%) of tert-butyl N-[(1R,3S)-3-hydroxy-1-methyl-butyl]carbamate (Intermediate RP6) and 1.82 g (47%) of tert-butyl N-[(1R,3R)-3-hydroxy-1-methyl-butyl]carbamate.

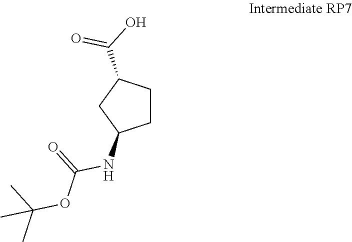 [SMILES: CC(C)(C)OC(=O)N[C@@H]1CC[C@@H](C(=O)O)C1]

### (1R,3R)-3-(tert-butoxycarbonylamino)cyclopentanecarboxylic acid

The title compound was prepared according to procedures described in WO2019/236631 starting from (1S)-(+)-2-azabicyclo[2.2.1]hept-5-en-3-one (10 g.) to give 7.79 g (37.1% over 4 steps) of (1R,3R)-3-(tert-butoxycarbonylamino)cyclopentanecarboxylic acid

### (Intermediate RP7)

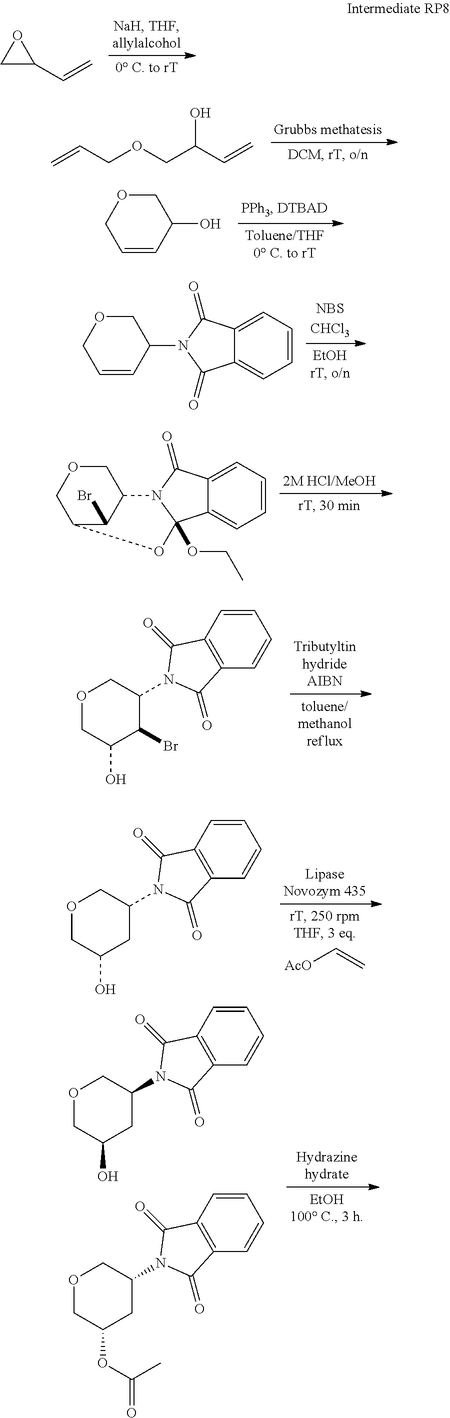

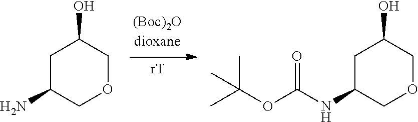

### tert-Butyl N-[(3S,5R)-5-hydroxytetrahydropyran-3-yl]carbamate

### (a) 1-Allyloxybut-3-en-2-ol

To a cold (0° C.) solution of 2-vinyloxirane (2 mL, 25 mmol) and prop-2-en-1-ol (3.4 mL, 50 mmol) in DMF (50 mL) was added carefully portion-wise sodium hydride (60% in mineral oil, 2 g, 50 mmol). After stirring at 0° C. for 30 min, the mixture was stirred at 50° C. o/n. The mixture was cooled to 0° C. and the reaction quenched by addition of 1N HCl-solution (100 mL) and stirred for 1 h, allowing the temperature to come to room temperature. The mixture was extracted with diethyl ether (3×100 mL). The combined organic extracts were washed with 10% w/w LiCl-solution (100 mL) and brine. The organic layer was separated, dried over sodium sulfate, filtered and concentrated under reduced pressure (bath temperature 35° C., 600 mbar). The resulting residue was purified by column chromatography (pentane/diethyl ether=95/5 to 1/1 v/v %) to give 1.38 g of the title compound (Yield: 43.0%) as a colourless oil.

### (b) 3,6-Dihydro-2H-pyran-3-ol

To a solution of 1-allyloxybut-3-en-2-ol (1.38 g, 10.8 mmol) in dichloromethane (100 mL) was added Grubbs 1^{st} generation catalysator (178 mg, 0.22 mmol) and the reaction mixture was stirred at room temperature o/n. The mixture was concentrated under reduced pressure (bath temperature 35° C., 600 mbar). The resulting residue was purified by column chromatography (pentane/diethyl ether=95/5 to 1/1 v/v %) to give 566 mg of the title compound (Yield: 52.0%) as a colourless oil.

### (c) 2-(3,6-Dihydro-2H-pyran-3-yl)isoindoline-1 3-dione

To a cold (0° C.) solution of 3,6-dihydro-2H-pyran-3-ol (380 mg, 3.8 mmol), phthalimide (373 mg, 2.53 mmol) and triphenylphosphine (798 mg, 3.03 mmol) in THE (15 mL) was added dropwise a solution of di-tert-butyl azodicarboxylate (698 mg, 3.03 mmol) in THE (4 mL). The reaction mixture was stirred at 0° C. for 30 min and then allowed to come to room temperature and stirred for 3 h. The mixture was evaporated under reduced pressure to give a yellow oil. The resulting residue was purified by column chromatography (dichloromethane/ethyl acetate=10/0 to 1/9 v/v %) to give 456 mg of the title compound (Yield: 79%).

### (d) tert-Butyl N-[(3S,5R)-5-hydroxytetrahydropyran-3-yl]carbamate (Intermediate RP8)

This compound was prepared in an analogous manner as described Intermediate RP4 steps b to h, using 2-(3,6-dihydro-2H-pyran-3-yl)isoindoline-1,3-dione to afford 220 mg of the title compound (˜90% e.e.).

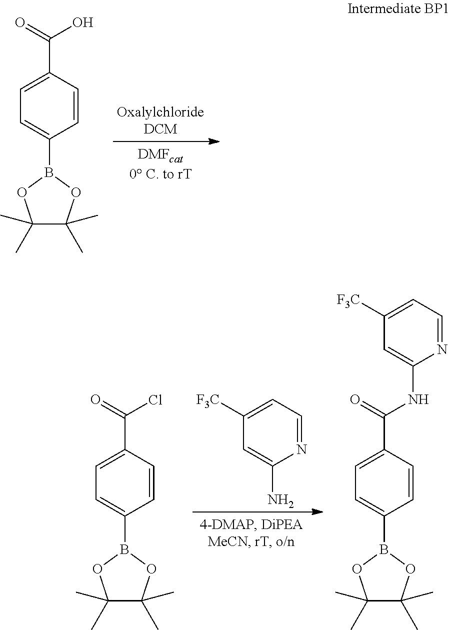

### 4-(4,4,5,5-Tetramethyl-1,3,2-dioxaborolan-2-yl)-N-[4-(trifluoromethyl)-2-pyridyl]benzamide Intermediate BP1

### (a) 4-(4,4,5,5-Tetramethyl-1,3,2-dioxaborolan-2-yl)benzoyl chloride

To a cold (0° C.) solution of 4-(4,4,5,5-tetramethyl-1,3,2-dioxaborolan-2-yl)benzoic acid (24.8 g, 100 mmol) in dichloromethane (300 mL) was added a catalytic amount of DMF. A solution of oxalyl chloride (12.9 mL, 150 mmol) was added dropwise. After stirring for 60 min at 0° C., the reaction mixture was allowed to warm to room temperature and the mixture was stirred o/n. The reaction mixture was concentrated to give 26.33 g of crude 4-(4,4,5,5-tetramethyl-1,3,2-dioxaborolan-2-yl)benzoyl chloride (yield: 99%).

### (b) 4-(4,4,5,5-Tetramethyl-1,3,2-dioxaborolan-2-yl)-N-[4-(trifluoromethyl)-2-pyridyl]benzamide Intermediate BP1

To a cold (0° C.) solution of 4-(4,4,5,5-tetramethyl-1,3,2-dioxaborolan-2-yl)benzoyl chloride (26.33 g, 100 mmol) in acetonitrile (300 mL) was subsequently added 4-(trifluoromethyl)pyridin-2-amine (19.45 g, 120 mmol) and 4-DMAP (14.66 g, 120 mmol) The mixture was stirred under nitrogen atmosphere at 0° C. and allowed to warm to room temperature overnight. The reaction mixture was concentrated in vacuo. The crude oily solids were then dissolved in dichloromethane (300 mL) and washed with 5% citric acid (3×, 300 mL), 5% NaHCO₃ (2×300 mL) and brine (200 mL). The organic layer was dried over sodium sulfate, filtered and concentrated in vacuo. The residue triturated in refluxing heptane (300 mL) for 1-2 hours. The mixture was filtrated and evaporated under reduced pressure. The residue was stirred in 6 N NaOH (140 mL) and THE (140 mL) at room temperature for 4 hrs. Then 250 mL EtOAc was added and the layers were separated. The organic layer was washed with water, 5% citric acid and brine, dried over sodium sulfate and concentrated in vacuo to give 24.4 g of 4-(4,4,5,5-tetramethyl-1,3,2-dioxaborolan-2-yl)-N-[4-(trifluoromethyl)-2-pyridyl]benzamide (Intermediate BP1) (yield: 62%) as off-white solids.

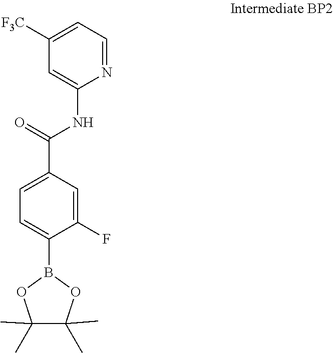 [SMILES: CC1(C)OB(c2ccc(C(=O)Nc3cc(C(F)(F)F)ccn3)cc2F)OC1(C)C]

### 3-Fluoro-4-(4,4,5,5-tetramethyl-1,3,2-dioxaborolan-2-yl)-N-[4-(trifluoromethyl)-2-pyridyl]benzamide (Intermediate BP2)

This compound was prepared in an analogous manner as described in Intermediate BP1, starting from 4-(trifluoromethyl)pyridine-2-amine and 3-fluoro-4-(4,4,5,5-tetramethyl-1,3,2-dioxaborolan-2-yl)benzoic acid, to afford the title compound (5.01 g, 69,7%).

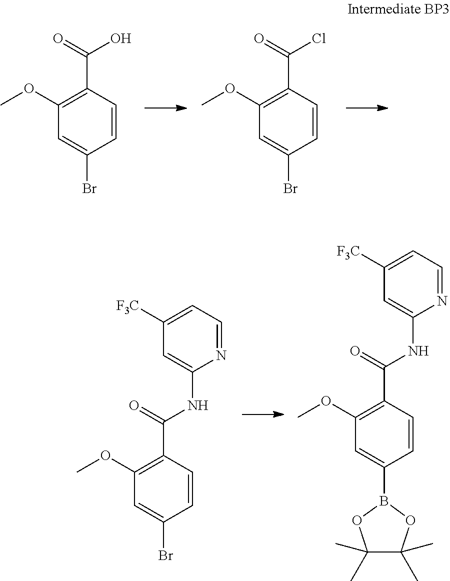 [SMILES: COc1cc(Br)ccc1C(=O)O ; COc1cc(Br)ccc1C(=O)Cl ; COc1cc(Br)ccc1C(=O)Nc1cc(C(F)(F)F)ccn1 ; COc1cc(B2OC(C)(C)C(C)(C)O2)ccc1C(=O)Nc1cc(C(F)(F)F)ccn1]

### 2-Methoxy-4-(4,4,5,5-tetramethyl-1,3,2-dioxaborolan-2-yl)-N-[4-(trifluoromethyl)-2-pyridyl]benzamide (Intermediate BP3)

### (a) 4-Bromo-2-methoxybenzoyl chloride

This compound was prepared in an analogous manner as described in Intermediate BP1 step a, starting from 4-bromo-2-methoxybenzoic acid to afford the title compound (5.75 g, quant. crude).

### (b) 4-Bromo-2-methoxy-N-[4-(trifluoromethyl)-2-pyridyl]benzamide

This compound was prepared in an analogous manner as described in Intermediate BP1 step b, starting from 4-bromo-2-methoxybenzoyl chloride and 4-(trifluoromethyl)pyridine-2-amine to afford the title compound (6.6 g, 81%).

### (c) 2-Methoxy-4-(4,4,5.5-tetramethyl-1,3.2-dioxaborolan-2-yl)-N-[4-(trifluoromethyl)-2-pyridyl]benzamide (Intermediate BP3)

To a solution of 4-bromo-2-methoxy-N-[4-(trifluoromethyl)-2-pyridyl]benzamide (6.15 g, 16.4 mmol) in dioxane (100 mL) was added bis(pinacolato)diboron (5.4 g, 21.3 mmol) and potassium acetate (3.22 g, 32.8 mmol). The reaction mixture was degassed with nitrogen. Subsequently Pd(dppf)Cl₂.CH₂Cl₂ complex (670 mg, 0.82 mmol) was added and the reaction mixture was stirred at 80° C. for 8 h. The mixture was cooled to room temperature and after addition of water extracted with ethyl acetate. The organic layers were combined, washed with brine, dried over sodium sulfate, filtered and concentrated under reduced pressure. The residue was purified by column chromatography using SiO₂ and ethyl acetate/heptane=1/1 v/v %. All fractions containing the title compound were collected and concentrated in vacuo to give 6.13 g of 2-methoxy-4-(4,4,5,5-tetramethyl-1,3,2-dioxaborolan-2-yl)-N-[4-(trifluoromethyl)-2-pyridyl]benzamide (Intermediate BP3) (yield: 88%).

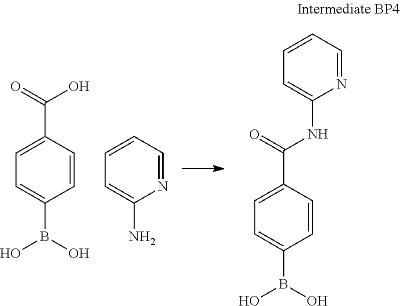

### [4-(2-Pyridylcarbamoyl)phenyl]boronic acid (Intermediate BP4)

4-Carboxyphenylboronic acid (15.0 g, 90.4 mmol) was suspended in toluene (180 mL) and DMF (277 μL, 3.61 mmol) was added. The reaction mixture was heated to 50 0C, at which point thionyl chloride (19.63 mL, 271 mmol) was added slowly (<5 min). The reaction mixture was heated to 60° C. and stirred for 8 h. After cooling to room temperature a white suspension occurred. The mixture was then concentrated under vacuum to remove solvent. Toluene was added and the mixture was concentrated to remove excess thionyl chloride. Pyridine (75 mL) was added and the slurry was cooled to 5° C. A solution of 2-aminopyridine (17.0 g, 180.8 mmol) in pyridine (30 mL) was added and the reaction mixture was slowly heated to 65° C. to 70° C. and stirred for 8 h. The reaction mixture was concentrated under vacuum to remove solvent. The residue was heated to 70° C. and water (10 mL) was added. After 1.5 h toluene (20 mL) was added followed by water (80 mL). The reaction mixture was cooled to 20° C., stirred for 72 h at room temperature. The suspension formed was filtered and washed with water (50 mL). The solids were re-suspended in water (50 mL) and stirred. The solids were filtered and washed with water (50 mL), dried in a vacuum oven at 40° C. yielding 12.7 g of [4-(2-pyridylcarbamoyl)phenyl]boronic acid (Intermediate BP4) as white solids (yield: 58%).

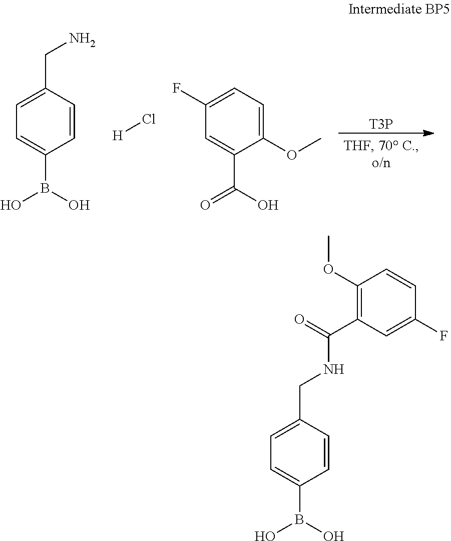

### [4-[[(5-fluoro-2-methoxy-benzoyl)amino]methyl]phenyl]boronic acid (Intermediate BP5)

To a suspension of 4-aminomethylphenylboronic acid hydrochloride (4.41 g, 23.5 mmol) and 5-fluoro-2-methoxybenzoic acid (4 g, 23.5 mmol) in anhydrous THE (100 mL), under a nitrogen atmosphere, was added successively, N,N-diisopropylethylamine (19.4 mL, 118 mmol) and 1-propanephosphonic acid cyclic anhydride (50 wt % in EtOAc, 23 mL, 35.3 mmol). The reaction mixture was heated under reflux at 70° C. o/n. The mixture was diluted with water and dichloromethane, then partitioned. The aqueous layer was extracted with DCM (2×). The combined organic extracts were filtered over a PE-filter and concentrated under reduced pressure. The residue was purified by column chromatography using SiO₂ and dichloromethane/methanol=99/1 to 95/5 v/v %. All fractions containing the title compound were collected and concentrated in vacuo to give 5.07 g of [4-[[(5-fluoro-2-methoxy-benzoyl)amino]methyl]phenyl]boronic acid (Intermediate BP5) (Yield 71%).

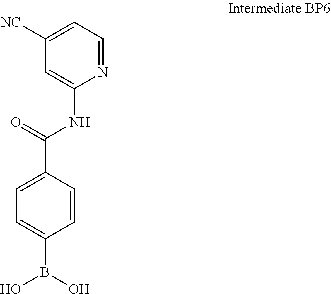 [SMILES: N#Cc1ccnc(NC(=O)c2ccc(B(O)O)cc2)c1]

### [4-[(4-cyano-2-pyridyl)carbamoyl]phenyl]boronic acid (Intermediate BP6)

This compound was prepared in an analogous manner as described in Intermediate BP1, starting from 2-aminopyridine-4-carbonitrile and 4-carboxyphenylboronic acid, to afford the title compound (4.8 g, 39%).

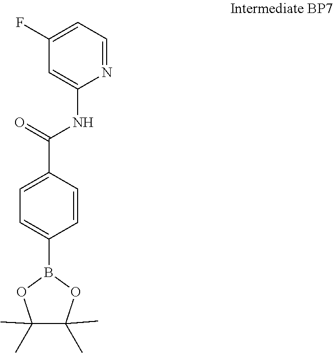 [SMILES: CC1(C)OB(c2ccc(C(=O)Nc3cc(F)ccn3)cc2)OC1(C)C]

### N-(4-Fluoro-2-pyridyl)-4-(4,4,5.5-tetramethyl-1,3,2-dioxaborolan-2-yl)benzamide (Intermediate BP7)

This compound was prepared in an analogous manner as described in Intermediate BP1, starting from 4-fluoropyridin-2-amine and 4-(4,4,5,5-tetramethyl-1,3,2-dioxaborolan-2-yl)benzoic acid, to afford the title compound (3.48 g, 33%).

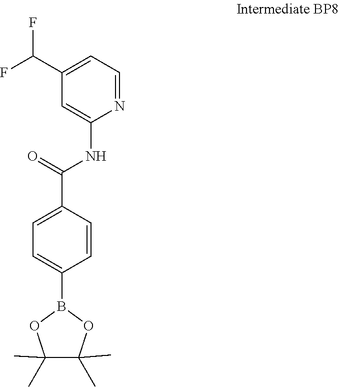 [SMILES: CC1(C)OB(c2ccc(C(=O)Nc3cc(C(F)F)ccn3)cc2)OC1(C)C]

### N-[4-(difluoromethyl)-2-pyridyl]-4-(4,4,5,5-tetramethyl-1,3,2-dioxaborolan-2-yl)benzamide (Intermediate BP8)

This compound was prepared in an analogous manner as described in Intermediate BP3, starting from 4-(difluoromethyl)pyridin-2-amine and 4-bromobenzoic acid, to afford the title compound (45 mg, 59%).

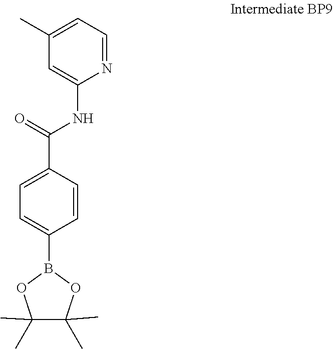 [SMILES: Cc1ccnc(NC(=O)c2ccc(B3OC(C)(C)C(C)(C)O3)cc2)c1]

### N-(4-Methyl-2-pyridyl)-4-(4,4,5,5-tetramethyl-1,3,2-dioxaborolan-2-yl)benzamide (Intermediate BP9)

This compound was prepared in an analogous manner as described in Intermediate BP1, starting from 2-amino-4-methylpyridine and 4-(4,4,5,5-tetramethyl-1,3,2-dioxaborolan-2-yl)benzoic acid, to afford the title compound (822 mg, 49%).

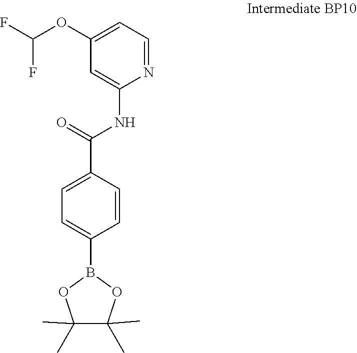 [SMILES: CC1(C)OB(c2ccc(C(=O)Nc3cc(OC(F)F)ccn3)cc2)OC1(C)C]

### N-[4-(Difluoromethoxy)-2-pyridyl]-4-(4,4,5,5-tetramethyl-1,3,2-dioxaborolan-2-yl)benzamide (Intermediate BP10)

This compound was prepared in an analogous manner as described in Intermediate BP3, starting from 4-(difluoromethoxy)pyridin-2-amine and 4-bromobenzoic acid, to afford the title compound (144 mg, 100%).

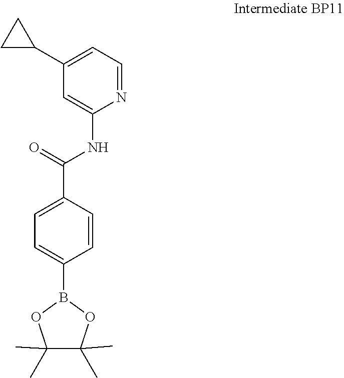 [SMILES: CC1(C)OB(c2ccc(C(=O)Nc3cc(C4CC4)ccn3)cc2)OC1(C)C]

### N-(4-Cyclopropyl-2-pyridyl)-4-(4,4,5.5-tetramethyl-1,3,2-dioxaborolan-2-yl)benzamide (Intermediate BP11)

This compound was prepared in an analogous manner as described in Intermediate BP3, starting from 4-cyclopropylpyridin-2-amine and 4-bromobenzoic acid, to afford the title compound (69 mg, 100%).

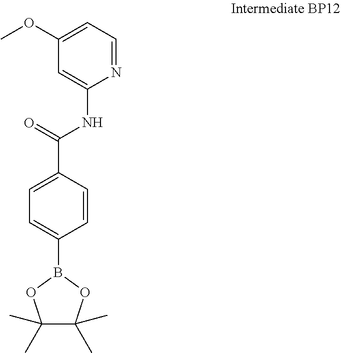 [SMILES: COc1ccnc(NC(=O)c2ccc(B3OC(C)(C)C(C)(C)O3)cc2)c1]

### N-(4-Methoxy-2-pyridyl)-4-(4,4,5,5-tetramethyl-1,3,2-dioxaborolan-2-yl)benzamide (Intermediate BP12)

This compound was prepared in an analogous manner as described in Intermediate BP1, starting from 2-amino-4-methoxypyridine and 4-(4,4,5,5-tetramethyl-1,3,2-dioxaborolan-2-yl)benzoic acid, to afford the title compound (900 mg, 51%).

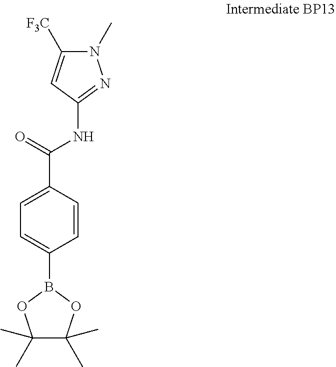

### N-[1-Methyl-5-(trifluoromethyl)pyrazol-3-yl]-4-(4,4,5,5-tetramethyl-1,3,2-dioxaborolan-2-yl)benzamide (Intermediate BP13)

This compound was prepared in an analogous manner as described in Intermediate BP1, starting from 1-methyl-5-(trifluoromethyl)-1H-pyrazol-3-amine and 4-(4,4,5,5-tetramethyl-1,3,2-dioxaborolan-2-yl)benzoic acid, to afford the title compound (1.9 g, 80%).

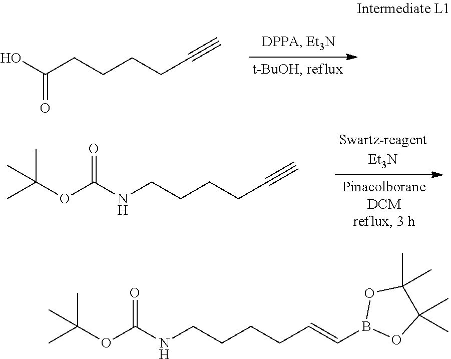

### tert-Butyl N-[(E)-6-(4,4,5,5-tetramethyl-1,3,2-dioxaborolan-2-yl)hex-5-enyl]carbamate (Intermediate L1)

### (a) tert-Butyl N-hex-5-ynylcarbamate

A solution of 6-heptynoic acid (0.800 mL, 6.32 mmol), diphenylphosphoryl azide (1.65 mL, 7.66 mmol), and Et₃N (1.75 mL, 12.6 mmol) in tert-BuOH (6.3 mL) was stirred at reflux for 48 h. The reaction mixture was cooled to rt and diluted with Et₂O (50 mL) and H₂O (50 mL). The layers were separated and the aqueous phase was extracted with Et₂O (2×50 mL). The combined organic extracts were washed with H₂O (100 mL) and brine (100 mL), dried over MgSO₄, filtered, and concentrated under reduced pressure to give a dark yellow oil. The oil was purified by chromatography on SiO₂ (15% EtOAc in hexanes) to give tert-butyl N-hex-5-ynylcarbamate (0.54 g, 43.3%) as a clear, colourless oil.

### (b) tert-Butyl N-[(E)-6-(4,4,5.5-tetramethyl-1,3,2-dioxaborolan-2-yl)hex-5-enyl]carbamate (Intermediate L1)

A solution of tert-butyl N-hex-5-ynylcarbamate (0.54 g, 2.74 mmol), ZrCp₂(H)CI (211 mg, 0.82 mmol), Et₃N (385 μL, 2.76 mmol), and pinacolborane (596 μL, 4.11 mmol) in dichloromethane (2.75 mL) was stirred at reflux for 3 h. The reaction mixture was cooled to room temperature and quenched by dropwise addition of methanol (3.15 mL). The organic solvents were evaporated under reduced pressure to give a cloudy white oil which was taken up in diethyl ether (5 mL) and filtered through a thin pad of boron-doped SiO₂. After “rinsing” with diethyl ether (25 mL) the filtrate was evaporated under reduced pressure to give 0.79 g (88.7%) a clear, colorless oil. ¹H-NMR showed the presence of reduced acetylene for ˜25% therefor the crude product was purified by flash chromatography on boron-doped silica gel in hexane/ethyl acetate=95/5 to 8/2 v/v % as eluent. The fractions containing the title compound were pooled and concentrated to obtain 481 mg of the title compound (yield: 54.0%).

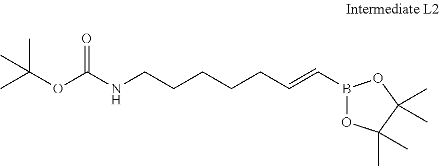 [SMILES: CC(C)(C)OC(=O)NCCCCC/C=C/B1OC(C)(C)C(C)(C)O1]

### tert-Butyl N-[(E)-7-(4,4,5,5-tetramethyl-1,3,2-dioxaborolan-2-yl)hept-6-enyl]carbamate (Intermediate L2)

This compound was prepared in an analogous manner as described in Intermediate L1 starting from 7-heptynoic acid to afford the title compound (742.6 mg, 28.9%).

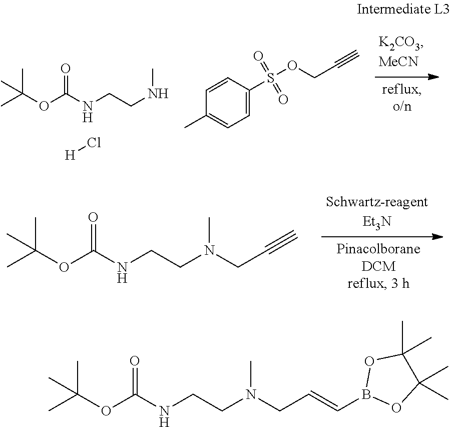

### tert-Butyl N-[2-[methyl-[(E)-3-(4,4,5,5-tetramethyl-1,3,2-dioxaborolan-2-yl)allyl]amino]ethyl]-carbamate (Intermediate L3)

### (a) tert-Butyl N-[2-[methyl(prop-2-ynyl)amino]ethyl]carbamate

To a solution of propargyl p-toluenesulfonate (1.05 g, 4.98 mmol) in acetonitril (25 mL) was subsequently added N-tert-butoxycarbonyl-2-methylamino-ethylamine hydrochloride (4.75 mmol) and potassium carbonate (1.31 g, 9.49 mmol) and the reaction mixture was refluxed for 4 h. Water and 5% sodium bicarbonate solution were added to the mixture and the mixture was extracted with ethyl acetate. The combined organic layers were washed with brine and concentrated under reduced pressure. The residue was purified by chromatography on SiO₂ (ethyl acetate) to give tert-butyl N-[2-[methyl(prop-2-ynyl)amino]ethyl]carbamate (0.88 g, 87.3%) as a clear, yellow oil.

### (b) tert-Butyl N-[2-[methyl-[(E)-3-(4,4,5,5-tetramethyl-1,3,2-dioxaborolan-2-yl)allyl]amino]ethyl]-carbamate (Intermediate L3)

A solution of tert-Butyl N-[2-[methyl(prop-2-ynyl)amino]ethyl]carbamate (0.88 g, 4.15 mmol), ZrCp2(H)CI (321 mg, 1.24 mmol), Et₃N (584 μL, 4.19 mmol), and pinacolborane (902 μL, 4.19 mmol) in dichloromethane (5 mL) was stirred at reflux for 3 h. The reaction mixture was cooled to room temperature and quenched by dropwise addition of methanol (5.3 mL). The organic solvents were evaporated under reduced pressure to give a cloudy white oil which was taken up in diethyl ether (5 mL) and filtered through a thin pad of boron-doped SiO₂. After “rinsing” with diethyl ether (25 mL) the filtrate was concentrated under reduced pressure to give a clear, yellow oil. The crude product was purified by flash chromatography on boron-doped silica gel in ethyl acetate as eluent. The fractions containing the title compound were pooled and concentrated to obtain 0.88 g of the title compound. (Yield: 62.4%)

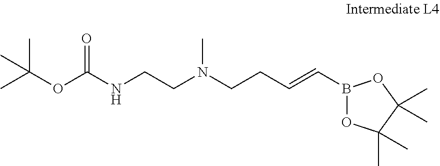 [SMILES: CN(CC/C=C/B1OC(C)(C)C(C)(C)O1)CCNC(=O)OC(C)(C)C]

### tert-Butyl N-[2-[methyl-[(E)-4-(4,4,5,5-tetramethyl-1,3,2-dioxaborolan-2-yl)but-3-enyl]amino]ethyl]carbamate (Intermediate L4)

This compound was prepared in an analogous manner as described in Intermediate L3, starting from N-tert-butoxycarbonyl-2-methylamino-ethylamine hydrochloride and 3-butynyl p-toluenesulfonate to afford the title compound (1.07 g, 52.6%).

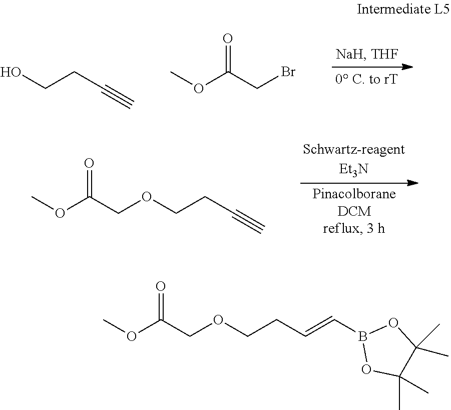

### Methyl 2-[(E)-4-(4,4,5,5-tetramethyl-1,3,2-dioxaborolan-2-yl)but-3-enoxylacetate (Intermediate L5)

### (a) Methyl 2-but-3-ynoxyacetate

To a cold (0° C.) suspension of NaH (60% dispersion in mineral oil, 428 mg, 10.7 mmol) in THE (7 mL) was added but-3-yn-1-ol (624 mg, 8.92 mmol mmol) in THE (1 mL). After stirring at room temperature for 30 min, the mixture was cooled to 0° C. and a solution of methyl 2-bromoacetate (1.36 g, 8.92 mmol) in THE (1 mL) was added dropwise. The reaction mixture was stirred o/n allowing the temperature to come to room temperature. Diethyl ether (25 mL) was added to the mixture and washed with water (25 mL). The water layer was extracted with diethyl ether (2×25 mL). The combined organic extracts were washed with brine, dried over sodium sulfate, filtered and concentrated under reduced pressure. The residue was purified by chromatography on SiO₂ (pentanes/diethyl ether=10/0 to 1/1 v/v %) to give methyl 2-but-3-ynoxyacetate (620 mg, 48%) as a clear, colourless oil.

### (b) Methyl 2-[(E)-4-(4,4,5,5-tetramethyl-1,3,2-dioxaborolan-2-yl)but-3-enoxy]acetate (Intermediate L5)

This compound was prepared in an analogous manner as described in Intermediate L1 step b, starting from methyl 2-but-3-ynoxyacetate to afford the title compound (480 mg, 42%).

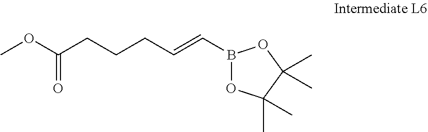

### Methyl(E)-6-(4,4,5,5-tetramethyl-1,3,2-dioxaborolan-2-yl)hex-5-enoate (Intermediate L6)

This compound was prepared in an analogous manner as described in Intermediate L1 step b, starting from methyl 5-hexynoate to afford the title compound (558 mg, 44%).

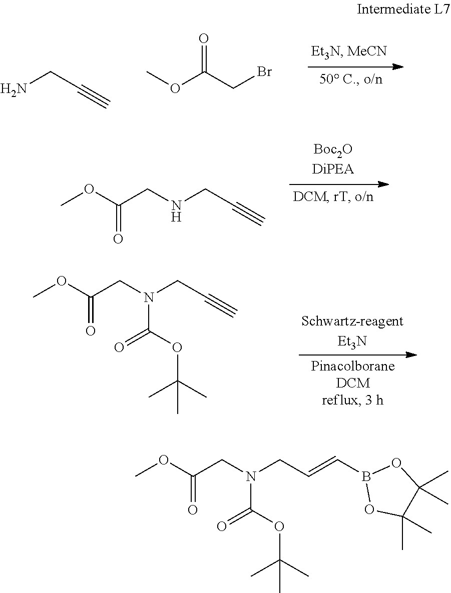

### Methyl 2-[tert-butoxycarbonyl-[(E)-3-(4,4,5.5-tetramethyl-1,3.2-dioxaborolan-2-yl)allyl]amino]acetate (Intermediate L7)

### (a) Methyl 2-(prop-2-ynylamino)acetate

To a solution of propargylamine (2.0 mL, 31.2 mmol) and triethylamine (4.8 mL, 34.4 mmol) was added slowly methyl bromoacetate (3.25 mL, 34.4 mmol). The reaction mixture was stirred at 50° C. for 12 h. The crude product, obtained after concentration of the mixture in vacuo, was purified by chromatography on SiO₂ (dichloromethane/methanol=10/0 to 95/5 v/v %) to give methyl 2-(prop-2-ynylamino)acetate (2.63 g, 67%) as an orange coloured oil.

### (b) Methyl 2-[tert-butoxycarbonyl(prop-2-ynyl)amino]acetate

To a cold (0° C.) solution of methyl 2-(prop-2ynylamino)acetate (1.50 g, 11.8 mmol) and DiPEA (3.90 mL, 23.6 mmol) in DCM (40 mL) was added Boc₂O (2.32 g, 10.6 mmol) and the mixture was stirred o/n. The mixture was washed with 1 N KHSO₄ (2×60 mL), brine, dried over sodium sulfate, filtered and concentrated in vacuo to yield the title compound (2.42 g, 86%).

### (c) Methyl 2-[tert-butoxycarbonyl-[(E)-3-(4,4,5,5-tetramethyl-1,3,2-dioxaborolan-2-yl)allyl]amino]acetate (Intermediate L7)

This compound was prepared in an analogous manner as described in Intermediate L1 step b, starting from methyl 2-[tert-butoxycarbonyl(prop-2-ynyl)amino]acetate to afford the title compound (831 mg, 53%).

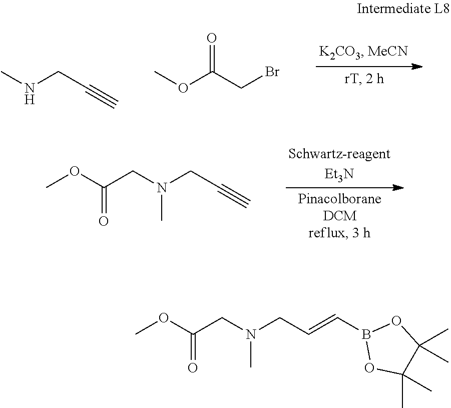

### Methyl 2-[methyl-[(E)-3-(4,4,5.5-tetramethyl-1,3.2-dioxaborolan-2-yl)allyl]amino]acetate (Intermediate L8)

This compound was prepared in an analogous manner as described in Intermediate L7, starting from N-methyl-N-prop-2ynyl-amine and methyl bromoacetate to afford the title compound (1.22 g, 64%).

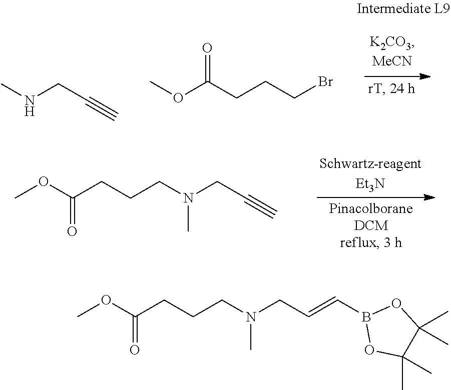

### Methyl 4-[methyl-[(E)-3-(4,4,5,5-tetramethyl-1,3,2-dioxaborolan-2-yl)allyl]amino]butanoate (Intermediate L9)

This compound was prepared in an analogous manner as described in Intermediate L7, starting from N-methyl-N-prop-2ynyl-amine and methyl 4-bromobutyrate to afford the title compound (344 mg, 16%).

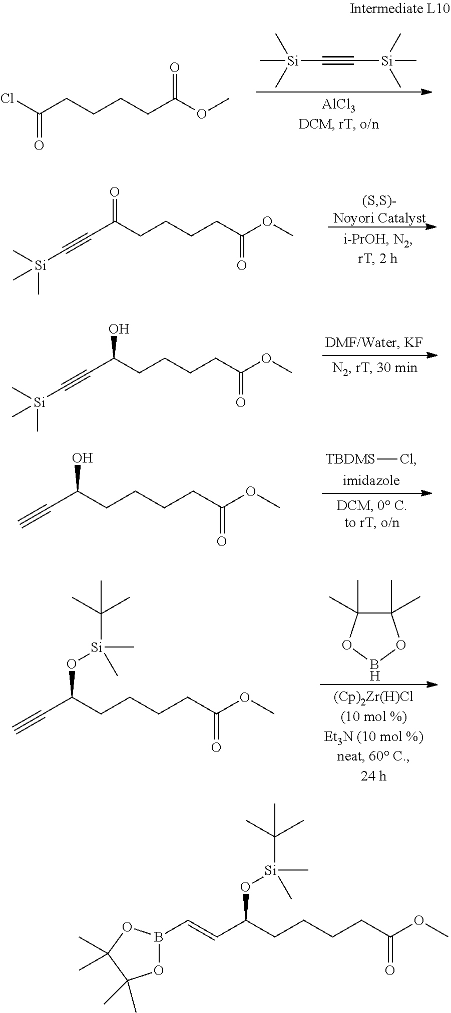

### Methyl(E,6S)-6-[tert-butyl(dimethyl)silyl]oxy-8-(4,4,5,5-tetramethyl-1,3,2-dioxaborolan-2-yl)oct-7-enoate (Intermediate L10)

### (a) Methyl 6-oxo-8-trimethylsilyl-oct-7-ynoate

A solution of bis(trimethylsilyl)acetylene (12.8 g, 96 mmol) and methyl 6-chloro-6-oxo-hexanoate (80 mmol) in dichloromethane (100 mL) was added dropwise to a suspension of aluminium chloride (12.8 g, 96 mmol) in dichloromethane (100 mL) at 0° C. and stirred for 2 h allowing the temperature to come to room temperature. The reaction mixture was quenched with ice and saturated aq. citric acid (100 mL) and extracted with diethyl ether. The combined organic layers were washed with brine, dried over sodium sulfate, filtered and concentrated in vacuo. The brown residue was purified by column chromatography (hexane/ethyl acetate=99:1 to 9/1 v/v %) on silica gel to afford the title compound (4.86 g, 25.3%) as a yellowish oil. Crude fractions were again purified by column chromatography (hexane/ethyl acetate=99:1 to 95/5 v/v %) on silica gel to afford the title compound (5.37 g, 27.9%) as a yellowish oil.

### (b) Methyl(6S)-6-hydroxy-8-trimethylsilyl-oct-7-ynoate

A mixture of (1 S,2S)-(+)-N-(4-toluenesulfonyl)-1,2-diphenylethylenediamine (295 mg, 0.806 mmol), dichloro(p-cymene)ruthenium(II)dimer (248 mg, 0.40 mmol) and potassium hydroxide (363 mg, 6.47 mmol) in dichloromethane (10 mL) was stirred at room temperature for 10 min. The solution was treated with water (10 mL) and the colour changed from orange to deep purple. The organic layer was separated and dried over MgSO₄, filtered and concentrated in vacuo. The residue was dissolved in dichloromethane (2 mL) and added to a solution of methyl 6-oxo-8-trimethylsilyl-oct-7-ynoate (4.86 g, 20.22 mmol) in degassed isopropanol (50 mL) at room temperature. After stirring o/n, the solution was recharged with same amount of pre-treaded Ru-cat. and stirred at room temperature for 2 h., concentrated under reduced pressure and the residue was filtered on silica gel (hexane/ethyl acetate=98/2 to 9/1 v/v %) to afford the title compound (4.27 g, 87.1%) as a yellow/orange oil.

### (c) Methyl(6S)-6-hydroxyoct-7-ynoate

Methyl(6S)-6-hydroxy-8-trimethylsilyl-oct-7-ynoate (4.27 g, 17.6 mmol) was dissolved in DMF (40 mL) and treated with a solution of potassium fluoride (2.05 g, 35.2 mmol) in water (5 mL) at room temperature. After 30 min, 1 M hydrochloric acid (50 mL) was added and the product was extracted with diethyl ether (3×50 mL). The combined organic layers were washed with brine (30 mL), dried over sodium sulfate and concentrated in vacuo. The residue was purified by column chromatography on silica gel (hexane/ethyl acetate=6/4 v/v %) affording methyl(6S)-6-hydroxyoct-7-ynoate (2.4 g, 80.0%) as a yellowish oil.

### (d) Methyl(6S)-6-[tert-butyl(dimethyl)silyl]oxyoct-7-ynoate

To a solution of methyl(6S)-6-hydroxyoct-7-ynoate (2.4 g, 14.1 mmol) in dichloromethane (25 mL) under nitrogen atmosphere was added at 0° C. tert-butyldimethylsilyl chloride (2.34 g, 15.51 mmol) and imidazole (1.92 g, 28.2 mmol). The mixture was stirred at room temperature o/n and water was added. The aqueous layer was separated over a PE-filter and the filtrate was concentrated under reduced pressure. The residue was purified by column chromatography on silica gel (hexane/ethyl acetate=99/1 to 9/1 v/v %) affording methyl(6S)-6-[tert-butyl(dimethyl)silyl]oxyoct-7-ynoate (3.79 g, 75.6% over two steps) as a colourless oil.

### (e) Methyl(E,6S)-6-[tert-butyl(dimethyl)silyl]oxy-8-(4,4,5.5-tetramethyl-1,3.2-dioxaborolan-2-yl)oct-7-enoate (Intermediate L10)

To an oven-dried 10 mL Schlenk-tube equipped with a magnetic stirring bar were added Schwartz's reagent (90.6 mg, 0.35 mmol), methyl(6S)-6-[tert-butyl(dimethyl)silyl]oxyoct-7-ynoate (1 g, 3.51 mmol), Et₃N (49 μL, 0.35 mmol) and pinacolborane (552 μL, 3.69 mmol), under an inert nitrogen atmosphere. The tube was then sealed and the mixture was stirred at 60° C. for 24 hours. The reaction was allowed to cool to room temperature, diluted with diethyl ether, passed through a pad of silica gel and concentrated under reduced pressure at room temperature. The crude mixture was purified by column chromatography using SiO₂ and hexane/ethyl acetate=99/1 to 9/1 v/v %. All fractions containing the title compound were collected and concentrated in vacuo to give 311.6 mg of the title compound (yield: 21.5%).

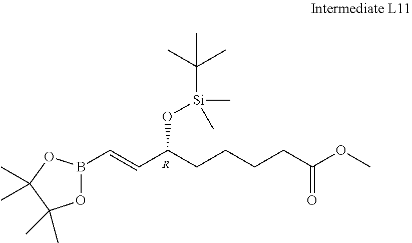

### Methyl(E,6R)-6-[tert-butyl(dimethyl)silyl]oxy-8-(4,4,5.5-tetramethyl-1,3.2-dioxaborolan-2-yl)oct-7-enoate (Intermediate L11)

This compound was prepared in an analogous manner as described in Intermediate L10, starting from methyl 6-oxo-8-trimethylsilyl-oct-7-ynoate and of (1R,2R)-(+)-N-(4-toluenesulfonyl)-1,2-diphenylethylenediamine to afford the title compound (295 mg, 20.4%).

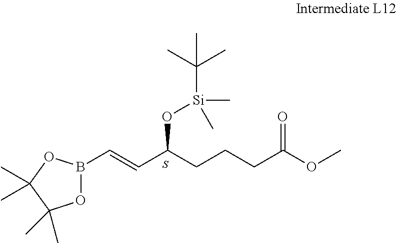

### Methyl(E,5S)-5-[tert-butyl(dimethyl)silyl]oxy-7-(4,4,5,5-tetramethyl-1,3,2-dioxaborolan-2-yl)hept-6-enoate (Intermediate L12)

This compound was prepared in an analogous manner as described in Intermediate L10, starting from methyl 5-oxo-7-trimethylsilyl-hept-6-ynoate and of (1 S,2S)-(+)-N-(4-toluenesulfonyl)-1,2-diphenylethylenediamine to afford the title compound (367.6 mg, 20.1%).

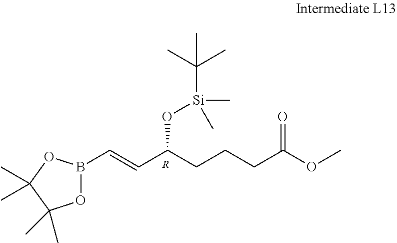

### Methyl(E,5R)-5-[tert-butyl(dimethyl)silyl]oxy-7-(4,4,5,5-tetramethyl-1,3,2-dioxaborolan-2-yl)hept-6-enoate (Intermediate L13)

This compound was prepared in an analogous manner as described in Intermediate L10, starting from methyl 5-oxo-7-trimethylsilyl-hept-6-ynoate and of (1R,2R)-(+)-N-(4-toluenesulfonyl)-1,2-diphenylethylenediamine to afford the title compound (400.9 mg, 21.8%).

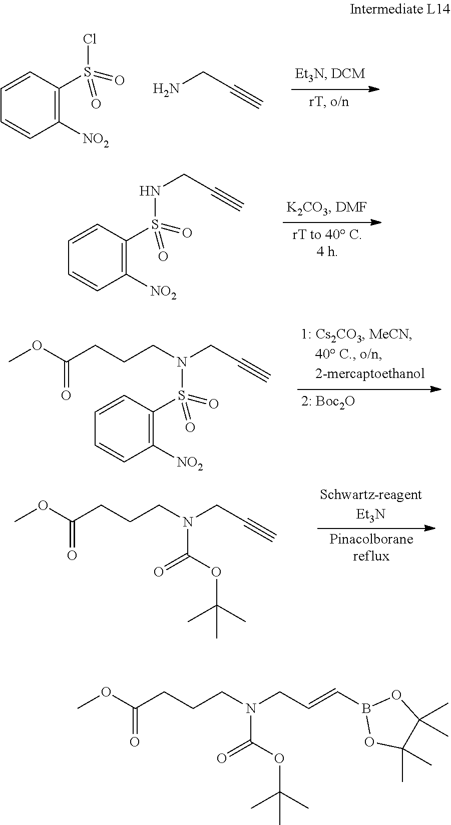

### Methyl 4-[tert-butoxycarbonyl-[(E)-3-(4,4,5,5-tetramethyl-1,3,2-dioxaborolan-2-yl)allyl]amino]butanoate (Intermediate L14)

### (a) 2-Nitro-N-prop-2-ynyl-benzenesulfonamide

To a solution of propargylamine (3.2 mL, 50 mmol) and triethylamine (17.5 mL, 125 mmol) in dichloromethane (100 mL) was added 2-nitrophenylsulfonyl chloride (10.5 g, 47.2 mmol). The reaction mixture was stirred at room temperature o/n. The mixture was washed, after addition of dichloromethane (100 mL), with 1N HCl-solution, water, brine, dried over sodium sulfate, filtered and concentrated under reduced pressure. The residue was triturated with ethyl acetate/heptane to give 2-nitro-N-prop-2-ynyl-benzenesulfonamide (9.57 g, 79%).

### (b) Methyl 4-[(2-nitrophenyl)sulfonyl-prop-2-ynyl-amino]butanoate

To a solution of 4-nitro-N-prop-2-ynyl-benzenesulfonamide (1 g, 4.16 mmol) and K₂CO₃ (1.15 g, 8.32 mmol) in DMF (12 mL) was added t-butyl-3-bromopropionate (0.9 g, 5 mmol) and the mixture was stirred at 40° C. for 4 h. The reaction mixture was diluted with EtOAc (100 mL) and washed with water (3×100 mL), brine and dried over sodium sulfate, filtered and concentrated in vacuo. The crude mixture was purified by column chromatography using SiO₂ and heptane/ethyl acetate=1/1 v/v %. All fractions containing the title compound were collected and concentrated in vacuo to give 1.5 g of the title compound (yield: 85%).

### (c) Methyl 4-[tert-butoxycarbonyl(prop-2-ynyl)amino]butanoate

To a solution of methyl 4-[(2-nitrophenyl)sulfonyl-prop-2-ynyl-amino]butanoate (0.950 g, 2.79 mmol) and cesium carbonate (1.82 g, 5.58 mmol) in MeCN (15 mL) was added 2-mercaptoethanol (235 μL, 3.35 mmol) This was heated to 40° C. for 1 d. Subsequently di-tert-butyl bicarbonate (0.245 g, 1.12 mmol) was added and stirred at rt for 1 h. After addition of ethyl acetate (100 mL) the mixture was washed with 5% aq. NaHCO₃-solution, brine, dried over sodium sulfate, filtered and concentrated in vacuo. The crude mixture was purified by column chromatography using SiO₂ and heptane/ethyl acetate=1/1 v/v %. All fractions containing the title compound were collected and concentrated in vacuo to give 0.73 g of the title compound (yield: 72%).

### (d) Methyl 4-[tert-butoxycarbonyl-[(E)-3-(4,4,5.5-tetramethyl-1,3.2-dioxaborolan-2-yl)allyl]-amino]butanoate (Intermediate L14)

This compound was prepared in an analogous manner as described in Intermediate L1 step b, starting from methyl 4-[tert-butoxycarbonyl(prop-2-ynyl)amino]butanoate to afford the title compound (304 mg, 28%).

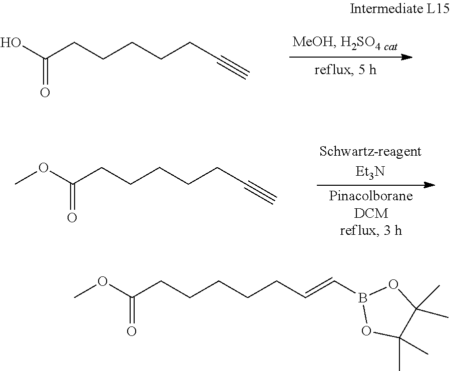

### Methyl(E)-8-(4.4.5.5-tetramethyl-1.3.2-dioxaborolan-2-yl)oct-7-enoate (Intermediate L15) (a) Methyl oct-7-ynoate

To a solution of oct-7-ynoic acid (1.82 g, 13 mmol) in methanol (15 ml) was added conc.-H₂SO₄ (4 drops). The reaction mixture was stirred for 5 h. at 70° C. After cooling of the mixture, ethyl acetate (150 mL) was added and the organic phase was washed with sat. aq. NaHCO₃-solution, water, brine, dried over sodium sulfate, filtered and concentrated under reduced pressure to give 1.86 g of methyl oct-7-ynoate (yield: 93%).

### (b) Methyl 2-[(E)-4-(4,4,5.5-tetramethyl-1,3,2-dioxaborolan-2-yl)but-3-enoxy]acetate (Intermediate L15)

This compound was prepared in an analogous manner as described in Intermediate L10 step e, starting from methyl oct-7-ynoate to afford the title compound (881 mg, 47%).

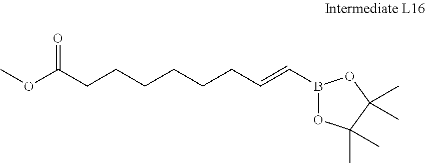

### Methyl(E)-9-(4,4,5,5-tetramethyl-1,3,2-dioxaborolan-2-yl)non-8-enoate (Intermediate L16)

This compound was prepared in an analogous manner as described in Intermediate L15, starting from non-8-ynoic acid to afford the title compound (785 mg, 45%).

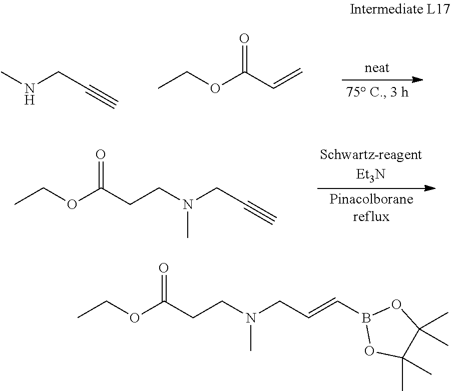

### Ethyl 3-[methyl-[(E)-3-(4,4,5,5-tetramethyl-1,3,2-dioxaborolan-2-yl)allyl]amino]propanoate (Intermediate L17)

### (a) Ethyl 3-[methyl(prop-2-ynyl)amino]propanoate

N-methyl propargylamine (1.0 g, 18.15 mmol) was added to ethyl acrylate (1.21 g, 12.1 mmol) followed by addition of acidic alumina (24.2 mmol, 2 eq.) and the mixture was stirred at 75° C. in a sealed tube for 3 h. The mixture was purified directly by column chromatography using SiO₂ and heptane/ethyl acetate=10/0 to 3/7 v/v %. All fractions containing the title compound were collected and concentrated in vacuo to give 2.41 g of the title compound (yield: 106%).

### (b) Ethyl 3-[methyl-[(E)-3-(4,4,5,5-tetramethyl-1,3,2-dioxaborolan-2-yl)allyl]amino]propanoate (Intermediate L17)

This compound was prepared in an analogous manner as described in Intermediate L10 step e, starting from ethyl 3-[methyl(prop-2-ynyl)amino]propanoate to afford the title compound (112 mg, 24%).

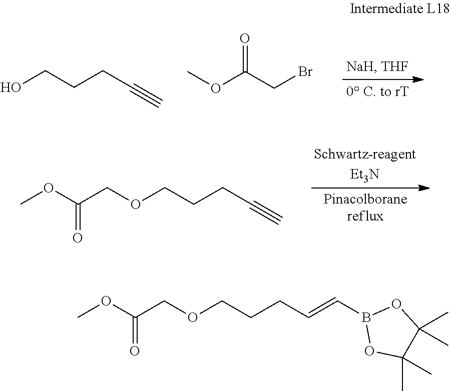

### Methyl 2-[(E)-5-(4,4,5.5-tetramethyl-1,3,2-dioxaborolan-2-yl)pent-4-enoxy]acetate (Intermediate L18)

### (a) Methyl 2-pent-4-ynoxyacetate

Sodium hydride (60% dispersion in mineral oil, 428 mg, 10.7 mmol) was suspended in THE (7 mL) and cooled to 0° C. Next, a solution of the 4-pentyn-1-ol (750 mg, 8.92 mmol) in THE (1 mL) was added dropwise. The reaction mixture was allowed to come to room temperature and stirred for 30 min. The resulting suspension was cooled to 0° C. again and methyl 2-bromoacetate (1.36 g, 8.92 mmol) in THE (1 mL) was added dropwise. The resulting mixture was allowed to come to room temperature and stirred o/n. The reaction mixture was diluted with diethyl ether (25 mL) followed by addition of sat. aq. ammonium chloride solution (10 mL). The aqueous layer was extracted twice with diethyl ether (10 mL). The combined organic layers were, dried over anhydrous sodium sulfate, filtered and concentrated in vacuo. (first 46° C., 750 mbar, followed by rt, 70 mbar for 15 minutes) to give an orange/brown liquid. The mixture was purified directly by column chromatography using SiO₂ and pentanes/diethyl ether=95/5 to 1/1 v/v %. All fractions containing the title compound were collected and concentrated in vacuo to give 480 mg of the title compound (yield: 34%).

### (b) Methyl 2-[(E)-5-(4,4,5,5-tetramethyl-1,3,2-dioxaborolan-2-yl)pent-4-enoxy]acetate (Intermediate L18)

This compound was prepared in an analogous manner as described in Intermediate L10 step e, starting from methyl 2-pent-4-ynoxyacetate to afford the title compound (430 mg, 49%).

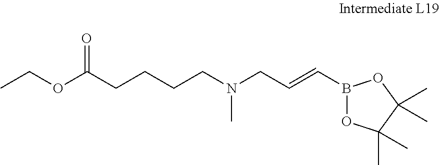 [SMILES: CCOC(=O)CCCCN(C)C/C=C/B1OC(C)(C)C(C)(C)O1]

### Ethyl 5-[methyl-[(E)-3-(4,4,5,5-tetramethyl-1,3,2-dioxaborolan-2-yl)allyl]amino]pentanoate (Intermediate L19)

This compound was prepared in an analogous manner as described in Intermediate L9, starting from N-methylprop-2-yn-1-amine and ethyl 5-bromopentanoate to afford the title compound (550 mg, 30%).

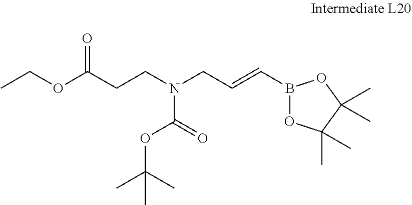 [SMILES: CCOC(=O)CCN(C/C=C/B1OC(C)(C)C(C)(C)O1)C(=O)OC(C)(C)C]

### Ethyl 3-[tert-butoxycarbonyl-[(E)-3-(4,4,5,5-tetramethyl-1,3,2-dioxaborolan-2-yl)allyl]amino]propanoate (Intermediate L20)

This compound was prepared in an analogous manner as described in Intermediate L17 step a and Intermediate 7 step b and c, starting from propargylamine and ethyl acrylate to afford the title compound (594 mg, 19%).

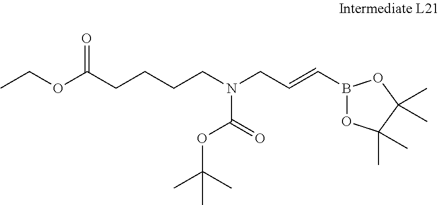 [SMILES: CCOC(=O)CCCCN(C/C=C/B1OC(C)(C)C(C)(C)O1)C(=O)OC(C)(C)C]

### Ethyl 5-[tert-butoxycarbonyl-[(E)-3-(4,4,5,5-tetramethyl-1,3,2-dioxaborolan-2-yl)allyl]amino]pentanoate (Intermediate L21)

This compound was prepared in an analogous manner as described in Intermediate L14, starting from propargylamine and ethyl 5-bromopentanoate to afford the title compound (537 mg, 25%).

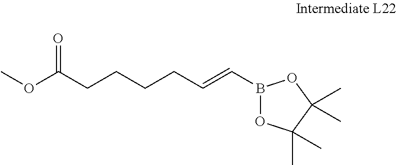

### Methyl(E)-7-(4,4,5,5-tetramethyl-1,3,2-dioxaborolan-2-yl)hept-6-enoate (Intermediate L22)

This compound was prepared in an analogous manner as described in Intermediate L15, starting from 6-heptynoic acid to afford the title compound (2.15 g, 25%).

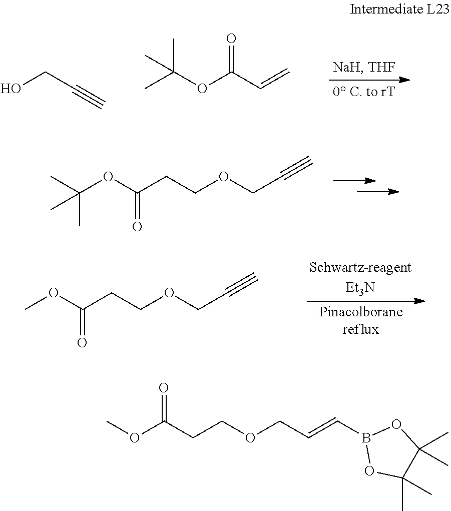

### Methyl 3-[(E)-3-(4,4,5.5-tetramethyl-1,3,2-dioxaborolan-2-yl)allyloxy]propanoate (Intermediate L23)

### (a) tert-Butyl 3-prop-2-ynoxypropanoate

To a solution of propargyl alcohol (1.36 mL, 23.4 mmol) in THE (20 mL) was added a small lump of sodium (˜20.08 mg, 0.874 mmol) and the reaction mixture was heated at 60° C. until complete solubilization (30-45 min) of sodium. The reaction mixture was cooled to room temperature and tert-butyl acrylate (2.29 mL, 15.6 mmol) in THE (3 mL) was added dropwise over 10 minutes. After completion of addition, the reaction mixture was stirred 3 h at room temperature. Water (25 mL) was added to the reaction mixture and the bi-phasic system was stirred 30 minutes at room temperature. The layers were separated and the aqueous phase was extracted with ethyl acetate (2×25 mL). The combined organic layers were washed with water (2×20 mL), brine, dried over Na₂SO₄, filtered and concentrated under reduced pressure to give 2.2 g (yield: 76%) of tert-butyl 3-prop-2-ynoxypropanoate as a colourless oil.

### (b) Methyl 3-prop-2-ynoxypropanoate

To a solution of tert-butyl 3-prop-2-ynoxypropanoate (2.2 g, 11.9 mmol) in dichloromethane (50 mL) was added trifluoroacetic acid (8 mL, 119 mmol). The reaction mixture was stirred at room temperature o/n. The mixture was concentrated and traces of trifluoroacetic acid were co-evaporated with toluene and DCM. The residue was purified by flash column chromatography using SiO₂ and dichloromethane/methanol=10/0 to 9/1 v/v %. All fractions containing the title compound were collected and concentrated in vacuo. The residue was dissolved in methanol (5 mL) and conc. H₂SO₄ (4 drops) was added. The reaction mixture was stirred at 70° C. o/n. After cooling of the mixture, ethyl acetate (150 mL) was added and the organic phase was washed with sat. aq. NaHCO₃-solution, water, brine, dried over sodium sulfate, filtered and concentrated under reduced pressure to give 1.34 g of methyl 3-prop-2-ynoxypropanoate (yield: 91%).

### (c) Methyl 3-[(E)-3-(4,4,5,5-tetramethyl-1,3,2-dioxaborolan-2-yl)allyloxy]propanoate (Intermediate L23)

This compound was prepared in an analogous manner as described in Intermediate L10 step e, starting from methyl 3-prop-2-ynoxypropanoate to afford the title compound (920 mg, 37%).

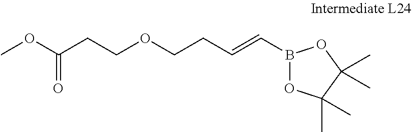

### Methyl 3-[(E)-4-(4,4,5,5-tetramethyl-1,3,2-dioxaborolan-2-yl)but-3-enoxy]propanoate (Intermediate L24)

This compound was prepared in an analogous manner as described in Intermediate L23, starting from but-3-yn-1-ol and tert-butyl acrylate to afford the title compound (1.32 g, 38%).

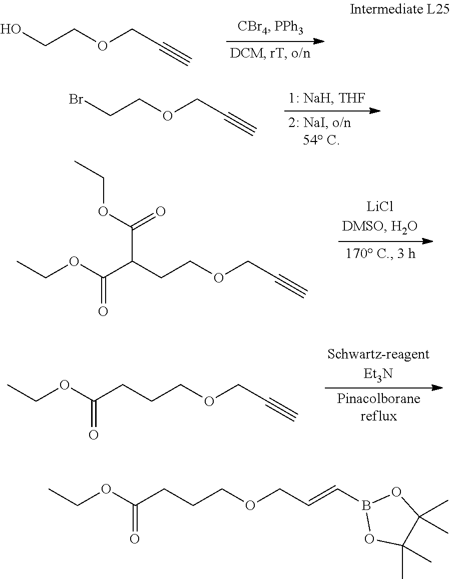

### Ethyl 4-[(E)-3-(4,4,5.5-tetramethyl-1,3.2-dioxaborolan-2-yl)allyloxy]butanoate (Intermediate L25)

(a) 3-(2-Bromoethoxy)prop-1-yne

Triphenylphosphine (3.93 g, 15 mmol) was added at room temperature, portion-wise to a stirred solution of carbon tetrabromide (4.97 g, 15 mmol) and 2-prop-2-ynoxyethanol (1.00 g, mmol) in DCM (33 mL). The reaction mixture was stirred at room temperature o/n. The mixture was concentrated and the residue was purified by column chromatography using SiO₂ and pentanes/diethyl ether=95/5 to 8/2 v/v %. All fractions containing the title compound were collected and concentrated in vacuo (500 mbar) to give 730 mg of the title compound (yield: 46%).

### (b) Diethyl 2-(2-prop-2-ynoxyethyl)propanedioate

NaH (60% dispersion in mineral oil, 122 mg, 3.0 mmol) was added carefully to a stirred solution of diethyl malonate (418 μL, 2.75 mmol) in THE (23 mL) at room temperature. The reaction mixture was stirred for 30 min after which a solution of 3-(2-bromoethoxy)prop-1-yne (450 mg, 2.75 mmol) in THE (4.5 mL) was added dropwise, followed by sodium iodide (405 mg, 2.75 mmol). The resulting mixture was stirred at 54° C. for 24 h. The mixture was diluted with water (25 mL) and diethyl ether (25 mL) was added. The organic phase was separated and the water layer was extracted with diethyl ether (2×20 mL). The combined organic extracts were washed with water (2×30 mL), brine (15 mL), dried (Na₂SO₄) filtered and concentrated in vacuo to give 500 mg of the title compound which was used directly in the next step.

### (c) Ethyl 4-prop-2-ynoxybutanoate

To a solution of diethyl 2-(2-prop-2-ynoxyethyl)propanedioate (635 mg, 2.62 mmol) in DMSO (1.3 mL) was added water (94 μL, 5.24 mmol) and lithium chloride (331 mg, 7.87 mmol). The resulting mixture was stirred at 170° C. for 3 h. Water (50 mL), brine (50 mL) and diethyl ether (50 mL) were added to the cooled mixture. After stirring 30 minutes at room temperature, the mixture was filtered over Decalite® and the layers of the filtrate were separated. The water layer was extracted with diethyl ether (2×25 mL). The combined organic extracts were washed with water (100 mL), brine (25 mL), dried (Na₂SO₄) filtered and concentrated in vacuo (500 mbar). The mixture was concentrated and the residue was purified by column chromatography using SiO₂ and pentanes/diethyl ether=99/1 v/v %. All fractions containing the title compound were collected and concentrated in vacuo (300 mbar) to give 103 mg of the title compound (yield: 46% taking into account content of 50%).

### (d) Ethyl 4-[(E)-3-(4,4,5,5-tetramethyl-1,3,2-dioxaborolan-2-yl)allyloxy]butanoate (Intermediate L25)

This compound was prepared in an analogous manner as described in Intermediate L10 step e, starting from ethyl 4-prop-2-ynoxybutanoate to afford the title compound (50 mg, 28%).

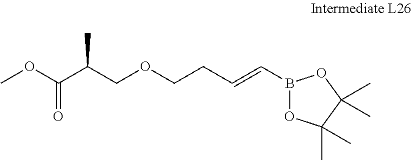

### Methyl(2R)-2-[(E)-3-(4,4,5,5-tetramethyl-1,3,2-dioxaborolan-2-yl)allyloxy]propanoate (Intermediate L26)

This compound was prepared in an analogous manner as described in Intermediate L18, starting from propargyl bromide and (R)-(+)-methyl lactate to afford the title compound (380 mg, 44%).

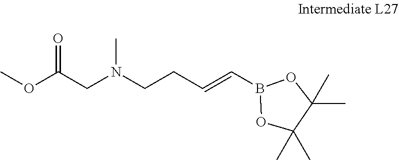

### Methyl 2-[methyl-[(E)-4-(4,4,5,5-tetramethyl-1,3,2-dioxaborolan-2-yl)but-3-enyl]amino]acetate (Intermediate L27)

This compound was prepared in an analogous manner as described in Intermediate L3, starting from 3-butynyl p-toluenesulfonate and sarcosine methyl ester, HCl to afford the title compound (372 mg, 37%).

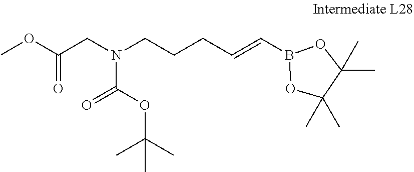

### Methyl 2-[tert-butoxycarbonyl-[(E)-5-(4,4,5,5-tetramethyl-1,3,2-dioxaborolan-2-yl)pent-4-enyl]amino]acetate (Intermediate L28)

This compound was prepared in an analogous manner as described in Intermediate L14, starting from 4-pentyn-1-ol and glycine methyl ester hydrochloride to afford the title compound (1.18 g, 88%).

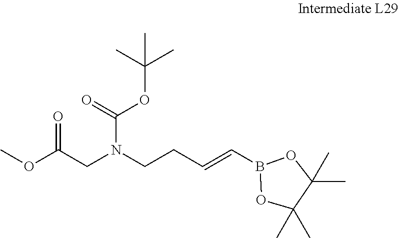

### Methyl 2-[tert-butoxycarbonyl-[(E)-4-(4,4,5,5-tetramethyl-1,3,2-dioxaborolan-2-yl)but-3-enyl]amino]acetate (Intermediate L29)

This compound was prepared in an analogous manner as described in Intermediate L14, starting from but-3-yn-1-ol and glycine methyl ester hydrochloride to afford the title compound (615 mg, 65%).

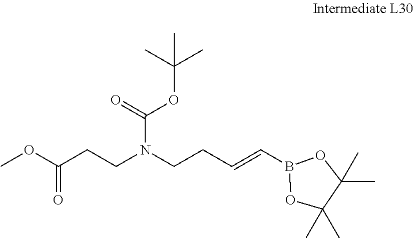

### Methyl 3-[tert-butoxycarbonyl-[(E)-4-(4,4,5.5-tetramethyl-1,3.2-dioxaborolan-2-yl)but-3-enyl]amino]propanoate (Intermediate L30)

This compound was prepared in an analogous manner as described in Intermediate L14, starting from but-3-yn-1-ol and methyl 3-aminopropanoate hydrochloride to afford the title compound (544 mg, 47%).

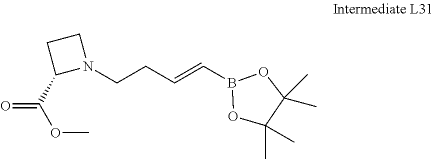 [SMILES: COC(=O)[C@@H]1CCN1CC/C=C/B1OC(C)(C)C(C)(C)O1]

### Methyl(2S)-1-[(E)-4-(4,4,5,5-tetramethyl-1,3,2-dioxaborolan-2-yl)but-3-enyl]azetidine-2-carboxylate (Intermediate L31)

This compound was prepared in an analogous manner as described in Intermediate L3 step a and Intermediate 10 step e, starting from methyl(2S)-azetidine-2-carboxylate hydrochloride and but-3-ynyl 4-methylbenzenesulfonate to afford the title compound (348 mg, 36%).

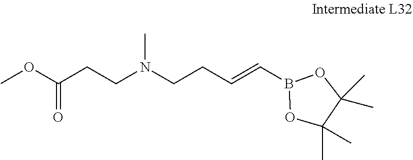

### Methyl 3-[methyl-[(E)-4-(4,4,5,5-tetramethyl-1,3,2-dioxaborolan-2-yl)but-3-enyl]amino]propanoate (Intermediate L32)

This compound was prepared in an analogous manner as described in Intermediate L3 step a and Intermediate 10 step e, starting from methyl 3-(methylamino)propanoate and but-3-ynyl 4-methylbenzenesulfonate to afford the title compound (574 mg, 40%).

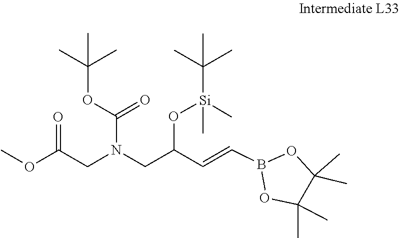

### Methyl 2-[tert-butoxycarbonyl-[(E)-2-[tert-butyl(dimethyl)silyl]oxy-4-(4,4,5.5-tetramethyl-1,3.2-dioxaborolan-2-yl)but-3-enyl]amino]acetate (Intermediate L33)

This compound was prepared in an analogous manner as described in Intermediate 7 and Intermediate L10, starting from the Weinreb amide of Boc-Gly-OH and TMS-acetylene to afford the title compound (241 mg, 35%).

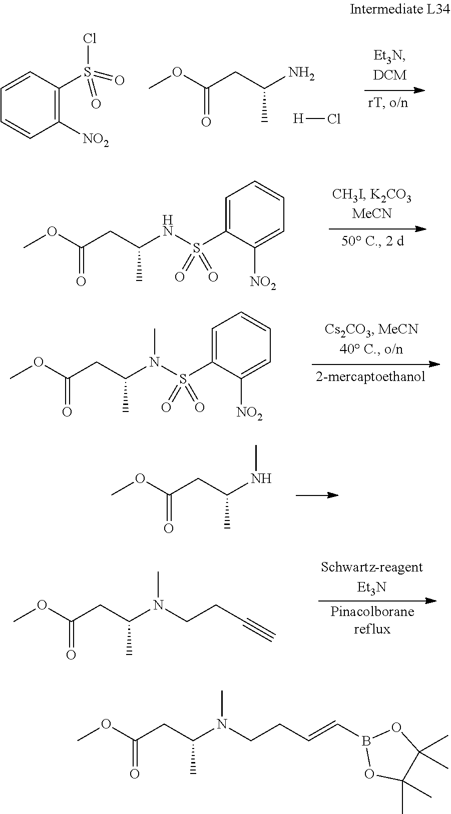

### Methyl(3R)-3-[methyl-[(E)-4-(4,4,5.5-tetramethyl-1,3.2-dioxaborolan-2-yl)but-3-enyl]amino]butanoate (Intermediate L34)

### (a) Methyl(3R)-3-[(2-nitrophenyl)sulfonylamino]butanoate

To a solution of (R)-3-amino-butyric acid methyl ester hydrochloride (3 g, 25.6 mmol) and triethylamine (8.5 mL, 64 mmol) in dichloromethane (75 mL) was added 2-nitrophenylsulfonyl chloride (5.8 g, 26.2 mmol). The reaction mixture was stirred at room temperature o/n. The mixture was washed, after addition of dichloromethane (75 mL), with 0.1 N HCl-solution, 5% aq. NaHCO₃-solution, water, brine, dried over sodium sulfate, filtered and concentrated under reduced pressure. The crude mixture was purified by column chromatography using SiO₂ and heptane/ethyl acetate=9/1 to 4/6 v/v %. All fractions containing the title compound were collected and concentrated in vacuo to give methyl(3R)-3-[(2-nitrophenyl)sulfonylamino]butanoate (5.6 g, 95%).

### (b) Methyl(3R)-3-[methyl-(2-nitrophenyl)sulfonyl-amino]butanoate

To a solution of methyl(3R)-3-[(2-nitrophenyl)sulfonylamino]butanoate (4.09 g, 12.9 mmol) and K₂CO₃ (3.57 g, 25.8 mmol) in acetonitrile (44 mL) was added methyl iodide (0.84 mL, 13.5 mmol) and the mixture was stirred at 50° C. for 2 d. The reaction mixture was diluted with EtOAc (100 mL) and washed with water (3×100 mL), brine and dried over sodium sulfate, filtered and concentrated in vacuo to give 4.13 g of the title compound (yield: 101%).

### (c) Methyl(3R)-3-(methylamino)butanoate

To a solution of methyl(3R)-3-[methyl-(2-nitrophenyl)sulfonyl-amino]butanoate (1.69 g, 5.34 mmol) and cesium carbonate (3.49 g, 10.7 mmol) in acetonitrile (20 mL) was added 2-mercaptoethanol (1.1 mL, 15.66 mmol) This was heated to 40° C. o/n. The reaction mixture was filtered and acidified with 2 N HCl-solution until pH 1 was reached. Then mixture was then loaded over a SCX-2 column (20 g). The column was washed with acetonitrile until the column was colourless, followed by a solvent switch to methanol and then the product was eluted with 2 N NH3/MeOH to yield methyl(3R)-3-(methylamino)butanoate (0.569 g, 81%).

### (d) Methyl(3R)-3-[methyl-[(E)-4-(4,4,5,5-tetramethyl-1,3,2-dioxaborolan-2-yl)but-3-enyl]amino]butanoate (Intermediate L34)

This compound was prepared in an analogous manner as described in Intermediate L3 step a and Intermediate 10 step e, starting from methyl(3R)-3-(methylamino)butanoate and but-3-ynyl 4-methylbenzenesulfonate to afford the title compound (980 mg, 57%).

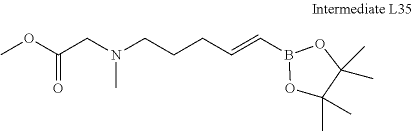

### Methyl 2-[methyl-[(E)-5-(4,4,5,5-tetramethyl-1,3,2-dioxaborolan-2-yl)pent-4-enyl]amino]acetate (Intermediate L35)

This compound was prepared in an analogous manner as described in Intermediate L3 step a and Intermediate 10 step e, starting from sarcosine methyl ester hydrochloride and pent-4-ynyl methanesulfonate to afford the title compound (760 mg, 37%).

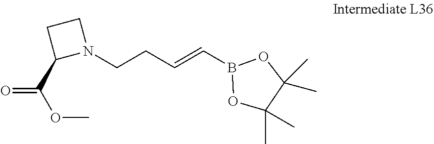 [SMILES: COC(=O)[C@H]1CCN1CC/C=C/B1OC(C)(C)C(C)(C)O1]

### Methyl(2R)-1-[(E)-4-(4,4,5,5-tetramethyl-1,3,2-dioxaborolan-2-yl)but-3-enyl]azetidine-2-carboxylate (Intermediate L36)

This compound was prepared in an analogous manner as described in Intermediate L3 step a and Intermediate 10 step e, starting from methyl(2R)-azetidine-2-carboxylate hydrochloride and but-3-ynyl 4-methylbenzenesulfonate to afford the title compound (1.1 g, 91%).

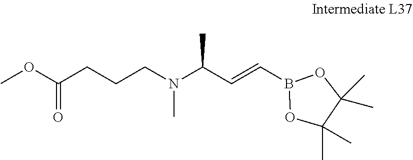

### Methyl 4-[methyl-[(E,1 S)-1-methyl-3-(4,4,5,5-tetramethyl-1,3,2-dioxaborolan-2-yl)allyl]amino]butanoate (Intermediate L37)

This compound was prepared in an analogous manner as described in Intermediate L3 step a and Intermediate 10 step e, starting from methyl 4-(methylamino)butanoate hydrochloride and [(1R)-1-methylprop-2-ynyl]4-methylbenzenesulfonate to afford the title compound (78 mg, 23%).

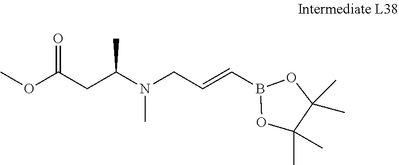

### Methyl(3R)-3-[methyl-[(E)-3-(4,4,5,5-tetramethyl-1,3,2-dioxaborolan-2-yl)allyl]amino]butanoate (Intermediate L38)

This compound was prepared in an analogous manner as described in Intermediate L34, starting from methyl(3R)-3-(methylamino)butanoate and propargyl p-toluenesulfonate to afford the title compound (215 mg, 24%).

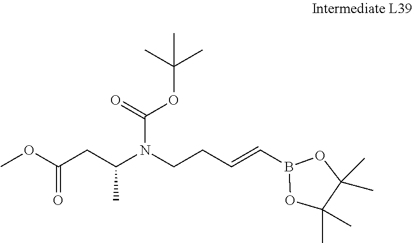

### Methyl(3R)-3-[tert-butoxycarbonyl-[(E)-4-(4,4,5,5-tetramethyl-1,3,2-dioxaborolan-2-yl)but-3-enyl]amino]butanoate (Intermediate L39)

This compound was prepared in an analogous manner as described in Intermediate L14, starting from but-3-yn-1-ol and (R)-3-amino-butyric acid methyl ester hydrochloride to afford the title compound (759 mg, 55%).

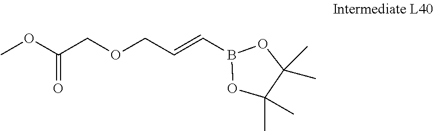

### Methyl 2-[(E)-3-(4,4,5,5-tetramethyl-1,3,2-dioxaborolan-2-yl)allyloxy]acetate (Intermediate L40)

This compound was prepared in an analogous manner as described in Intermediate L18, starting from propargyl alcohol and methyl bromoacetate to afford the title compound (359 mg, 41%).

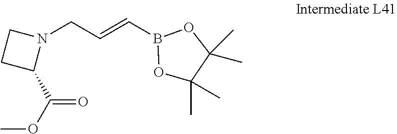

### Methyl(2S)-1-[(E)-3-(4,4,5.5-tetramethyl-1,3.2-dioxaborolan-2-yl)allyl]azetidine-2-carboxylate (Intermediate L41)

This compound was prepared in an analogous manner as described in Intermediate L3 step a and Intermediate 10 step e, starting from methyl(2S)-azetidine-2-carboxylate hydrochloride and prop-2-ynyl 4-methylbenzenesulfonate to afford the title compound (1.86 g, 80%).

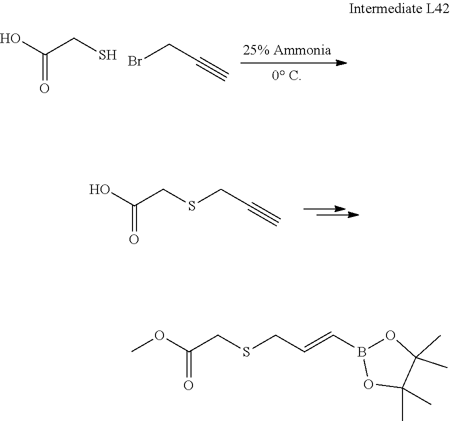

### Methyl 2-[(E)-3-(4,4,5,5-tetramethyl-1,3,2-dioxaborolan-2-yl)allyl]sulfanylacetate (Intermediate L42)

### (a) 2-prop-2-ynylsulfanylacetic acid

A solution of propargyl bromide in toluene (18.4 mL, 213 mmol) was added to a cold (0 15° C.) solution of mercaptoacetic acid (13.08 g, 142 mmol) in aqueous ammonia (24%, 250 mL). The reaction mixture was stirred at 0° C. for 40 min. The solution was concentrated, filtered and a sat. aq. NaHCO₃-solution was added. The solution was washed with dichloromethane. The aqueous phase was carefully acidified with concentrated HCl and extracted with dichloromethane. The organic phase was separated over a PE-filter and concentrated under reduced pressure giving 13.53 g of 2-prop-2-ynylsulfanylacetic acid as a slightly green oil which crystalized slowly to form off-white crystals (Yield: 73.2%).

### (b) 2-prop-2-ynylsulfanylacetic acid

To a solution of 2-prop-2-ynylsulfanylacetic acid (13.53 g, 104 mmol) in methanol (150 mL) was added 10 drops of H₂SO₄ (conc). The reaction mixture was stirred at reflux for 3 h. The mixture was concentrated under reduced pressure and ethyl acetate was added. The organic was washed carefully with 5% aq. NaHCO₃-solution, brine, dried over sodium sulfate, filtered and concentrated under reduced pressure to give 14.11 g of the title compound as a slightly brown oil (yield: 94.1%).

### (c) Methyl 2-[(E)-3-(4,4,5,5-tetramethyl-1,3,2-dioxaborolan-2-yl)allyl]sulfanylacetate (Intermediate L42)

This compound was prepared in an analogous manner as described in Intermediate 10 step e, starting from 2-prop-2-ynylsulfanylacetic acid to afford the title compound (455 mg, 25%).

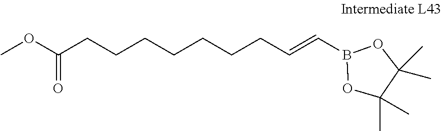

### Methyl(E)-10-(4,4,5,5-tetramethyl-1,3,2-dioxaborolan-2-yl)dec-9-enoate (Intermediate L43)

This compound was prepared in an analogous manner as described in Intermediate L15, starting from dec-9-ynoic acid to afford the title compound (801 mg, 39%).

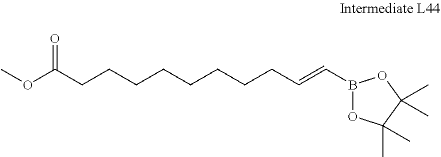

### Methyl(E)-11-(4,4,5,5-tetramethyl-1,3,2-dioxaborolan-2-yl)undec-10-enoate (Intermediate L44)

This compound was prepared in an analogous manner as described in Intermediate L15, starting from undec-1 0-ynoic acid to afford the title compound (278 mg, 14%).

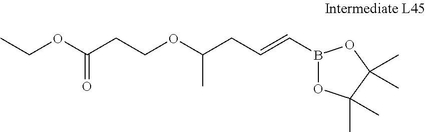 [SMILES: CCOC(=O)CCOC(C)C/C=C/B1OC(C)(C)C(C)(C)O1]

### Ethyl 3-[(E)-1-methyl-4-(4,4,5,5-tetramethyl-1,3,2-dioxaborolan-2-yl)but-3-enoxy]Propanoate (Intermediate L45)

This compound was prepared in an analogous manner as described in Intermediate L23, starting from 4-pentyn-2-ol and tert-butyl acrylate to afford the title compound (1.08 g, 87%).

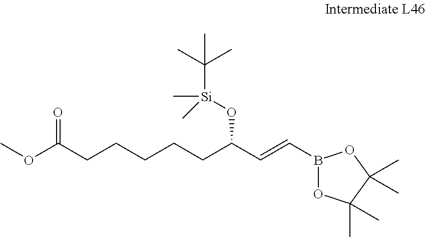

### Methyl(E,7S)-7-[tert-butyl(dimethyl)silyl]oxy-9-(4,4,5.5-tetramethyl-1,3.2-dioxaborolan-2-yl)non-8-enoate (Intermediate L46)

This compound was prepared in an analogous manner as described in Intermediate L10, starting from ethyl 7-chloro-7-oxo-heptanoate and of (1 S,2S)-(+)-N-(4-toluenesulfonyl)-1,2-diphenylethylenediamine to afford the title compound (477 mg, 22.2%).

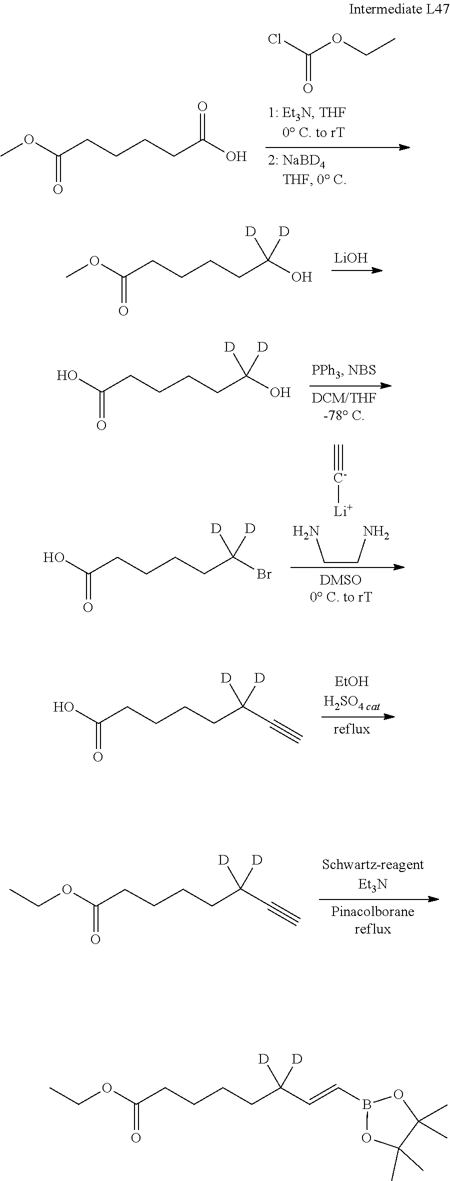

### Ethyl(E)-6,6-dideuterio-8-(4,4,5,5-tetramethyl-1,3,2-dioxaborolan-2-yl)oct-7-enoate (Intermediate L47)

### (a) Methyl 6,6-dideuterio-6-hydroxy-hexanoate

To a cold (0° C.) solution of adipic acid monomethyl ester (958 mg, 5.98 mmol) and triethylamine (917 μL, 6.58 mmol) in THF (9.5 mL) was added drop-wise solution of ethyl chloroformate (629 μL, 6.58 mmol) in THF (7.0 mL). The mixture was stirred for 1 h. allowing the temperature to come to room temperature. The mixture was filtrated and the residue washed with THF (5 mL). The combined filtrates were added dropwise to a solution of NaBD₄ (500 mg, 11.9 mmol) in water (14 mL) at 0° C. The reaction mixture was stirred for 1 h. The mixture was acidified with 2N aq. HCl-solution (10 mL) to adjust the pH to 3-4 and diethyl ether was added (20 mL). The resulting mixture was stirred 30 minutes at room temperature. The water layer was separated and extracted with diethyl ether (2×10 mL). The combined organic layers were washed with 0.5N NaOH (2×20 mL), water (20 mL) and brine (5.0 mL), dried (Na₂SO₄) and concentrated under reduced pressure. The crude product was purified by column chromatography using SiO₂ and pentane/diethyl ether=4/1 to 0/10 v/v %. All fractions containing the title compound were collected and concentrated in vacuo to give methyl 6,6-dideuterio-6-hydroxy-hexanoate (280 mg, 32%).

### (b) 6,6-Dideuterio-6-hydroxy-hexanoic acid

Methyl 6,6-dideuterio-6-hydroxy-hexanoate (280 mg, 1.89 mmol) was dissolved in THF/water=1/1 v/v % (18 mL) and subsequently lithium hydroxide (50 mg, 2.08 mmol) was added. The mixture was stirred at room temperature o/n. Ethyl acetate (50 mL) and water (were added) and the pH of the mixture was adjusted to pH<3 by addition of 2M HCl-solution. The organic phase was separated, washed with water, brine, dried over sodium sulfate, filtered and concentrated under reduced pressure to give 230 mg of 6,6-dideuterio-6-hydroxy-hexanoic acid (yield: 91%).

### (c) 6-Bromo-6,6-dideuterio-hexanoic acid

Triphenylphosphine (563 mg, 2.15 mmol) was dissolved in dichloromethane (8 mL) and cooled to −78° C. N-Bromosuccinimide (382 mg, 2.15 mmol) was added in one portion to the mixture and stirring was continued for 30 minutes at −78° C. Next, 6,6-dideuterio-6-hydroxy-hexanoic acid (230 mg, 1.72 mmol) dissolved in dichloromethane (8 mL) was added dropwise and the mixture was stirred for 45 min at −78° C. allowing to come to room temperature. The mixture was diluted with water and thoroughly stirred, 15 minutes at rt. The layers were separated and the water layer was extracted with dichloromethane (2×10 mL). The organic layers were combined and washed with 10% aq. Na₂S₂O₄-solution (20 mL), 0.2N aq. NaOH-solution. 2N aq. HCl-solution was added to the alkaline water layer to adjust pH<3. This water layer was extracted with dichloromethane. The combined organic layers were filtered over a PE filter and concentrated under reduced pressure to afford 230 mg of the title compound (yield: 54%).

### (d) 6.6-Dideuteriooct-7-ynoic acid

A solution of 6-bromo-6,6-dideuterio-hexanoic acid (405 mg, 2.05 mmol) in DMSO (1 mL) was added dropwise to a cold (0° C.) suspension of lithium acetylide ethylenediamine complex (560 mg, 6.09 mmol) in DMSO (2 mL). The resulting mixture was allowed to come to room temperature and stirred 1.5 h. The mixture was cautiously poured, at 0° C., in a mixture of ice-water (35 mL) and brine (20 mL) and stirred 30 min at 0° C. At this temperature, 2 N aq. HCl-solution (10 mL) was added followed by ethyl acetate (20 mL) and after stirring 30 minutes at room temperature, the layers were separated. The water layer was extracted with ethyl acetate (2×50 mL). The combined organic layers were washed with brine (25 mL), dried (Na₂SO₄), filtered and concentrated in vacuo. The crude product was purified by column chromatography using SiO₂ and heptane/ethyl acetate=95/5 to 1/4 v/v %. All fractions containing the title compound were collected and concentrated in vacuo to give 6,6-dideuteriooct-7-ynoic acid (200 mg, 69%).

### (e) Ethyl(E)-6.6-dideuterio-8-(4,4,5.5-tetramethyl-1,3.2-dioxaborolan-2-yl)oct-7-enoate (Intermediate L47)

This compound was prepared in an analogous manner as described in Intermediate L10, starting from ethyl 6,6-dideuteriooct-7-ynoate to afford the title compound (320 mg, 86%).

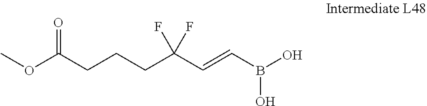

### [(E)-3.3-Difluoro-7-methoxy-7-oxo-hept-1-enyl]boronic acid (Intermediate L48)

This compound was prepared according to procedures described in Org. Lett. (2020) 22, 2991-2994 afford the title compound.

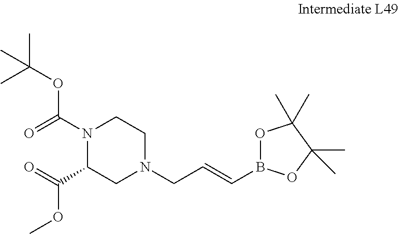 [SMILES: COC(=O)[C@H]1CN(C/C=C/B2OC(C)(C)C(C)(C)O2)CCN1C(=O)OC(C)(C)C]

### O1-tert-butyl 02-methyl(2R)-4-[(E)-3-(4,4,5,5-tetramethyl-1,3,2-dioxaborolan-2-yl)allyl]piperazine-1,2-dicarboxylate (Intermediate L49)

This compound was prepared in an analogous manner as described in Intermediate L3 step a and Intermediate 10 step e, starting from 1-tert-butyl 2-methyl(2R)-piperazine-1,2-dicarboxylate and propargyl p-toluenesulfonate to afford the title compound (300 mg, 72%).

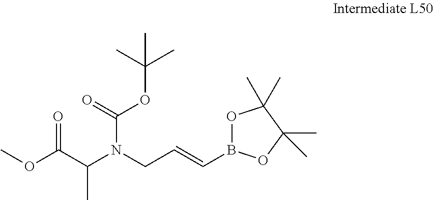

### Methyl 2-[tert-butoxycarbonyl-[(E)-3-(4,4,5,5-tetramethyl-1,3,2-dioxaborolan-2-yl)allyl]amino]propanoate (Intermediate L50)

This compound was prepared in an analogous manner as described in Intermediate L3 step a and Intermediate 10 step e, starting from Boc-D-Ala-OMe and propargyl bromide to afford the title compound (520 mg, 40%).

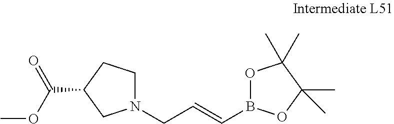

### Methyl(3R)-1-[(E)-3-(4,4,5,5-tetramethyl-1,3,2-dioxaborolan-2-yl)allyl]pyrrolidine-3-carboxylate (Intermediate L51)

This compound was prepared in an analogous manner as described in Intermediate L3 step a and Intermediate 10 step e, starting from methyl(3R)-pyrrolidine-3-carboxylate hydrochloride and propargyl p-toluenesulfonate to afford the title compound (410 mg, 40%).

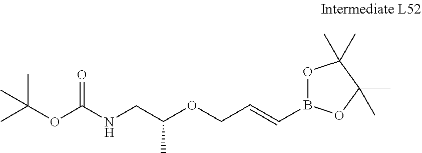 [SMILES: C[C@H](CNC(=O)OC(C)(C)C)OC/C=C/B1OC(C)(C)C(C)(C)O1]

### tert-Butyl N-[(2R)-2-[(E)-3-(4,4,5,5-tetramethyl-1,3,2-dioxaborolan-2-yl)allyloxy]propyl]carba-mate (Intermediate L52)

This compound was prepared in an analogous manner as described in Intermediate L3 step a and Intermediate 10 step e, starting from N-Boc-(R)-1-amino-2-propanol and propargyl bromide to afford the title compound (578 mg, 41%).

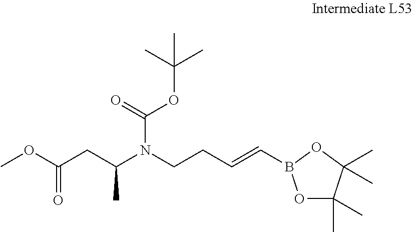

### Methyl(3S)-3-[tert-butoxycarbonyl-[(E)-4-(4,4,5.5-tetramethyl-1,3,2-dioxaborolan-2-yl)but-3-enyl]amino]butanoate (Intermediate L53)

This compound was prepared in an analogous manner as described in Intermediate L14, starting from but-3-yn-1-ol and (S)-3-amino-butyric acid methyl ester hydrochloride to afford the title compound (600 mg, 50%).

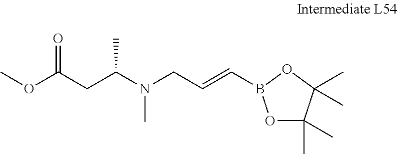

### Methyl(3S)-3-[methyl-[(E)-3-(4,4,5,5-tetramethyl-1,3,2-dioxaborolan-2-yl)allyl]amino]butanoate (Intermediate L54)

This compound was prepared in an analogous manner as described in Intermediate L34, starting from (S)-3-amino-butyric acid methyl ester hydrochloride and propargyl p-toluenesulfonate to afford the title compound (630 mg, 95%).

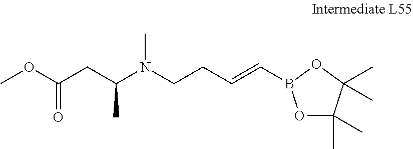

### Methyl(3S)-3-[methyl-[(E)-4-(4,4,5,5-tetramethyl-1,3,2-dioxaborolan-2-yl)but-3-enyl]amino]bu-tanoate (Intermediate L55)

This compound was prepared in an analogous manner as described in Intermediate L34, starting from (S)-3-amino-butyric acid methyl ester hydrochloride and 3-butynyl p-toluenesulfonate to afford the title compound (350 mg, 35%).

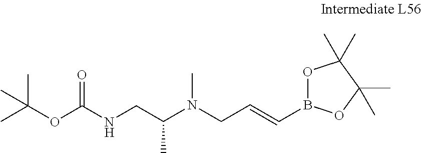 [SMILES: C[C@H](CNC(=O)OC(C)(C)C)N(C)C/C=C/B1OC(C)(C)C(C)(C)O1]

### tert-Butyl N-[(2R)-2-[methyl-[(E)-3-(4,4,5.5-tetramethyl-1,3,2-dioxaborolan-2-yl)allyl]amino]propyl]carbamate (Intermediate L56)

This compound was prepared in an analogous manner as described in Intermediate L10 step e, starting from tert-butyl N-[(2R)-2-[methyl(prop-2-ynyl)amino]propyl]carbamate to afford the title compound (272 mg, 87%).

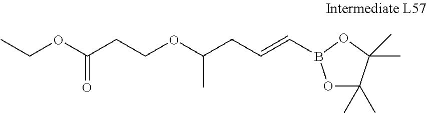 [SMILES: CCOC(=O)CCOC(C)C/C=C/B1OC(C)(C)C(C)(C)O1]

### Ethyl 3-[(E)-4-(4,4,5.5-tetramethyl-1,3.2-dioxaborolan-2-yl)but-3-enoxy]butanoate (Intermediate L57)

This compound was prepared in an analogous manner as described in Intermediate L23, starting from but-3-yn-1-ol and tert-butyl crotonate to afford the title compound (380 mg, 34%).

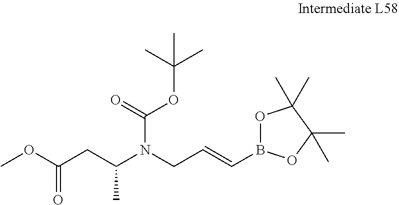

### Methyl(3R)-3-[tert-butoxycarbonyl-[(E)-3-(4,4,5.5-tetramethyl-1,3,2-dioxaborolan-2-yl)allyl]amino]butanoate (Intermediate L58)

This compound was prepared in an analogous manner as described in Intermediate L34, starting from but-3-yn-1-ol and (R)-3-aminobutyric acid methyl ester hydrochloride to afford the title compound (387 mg, 16%).

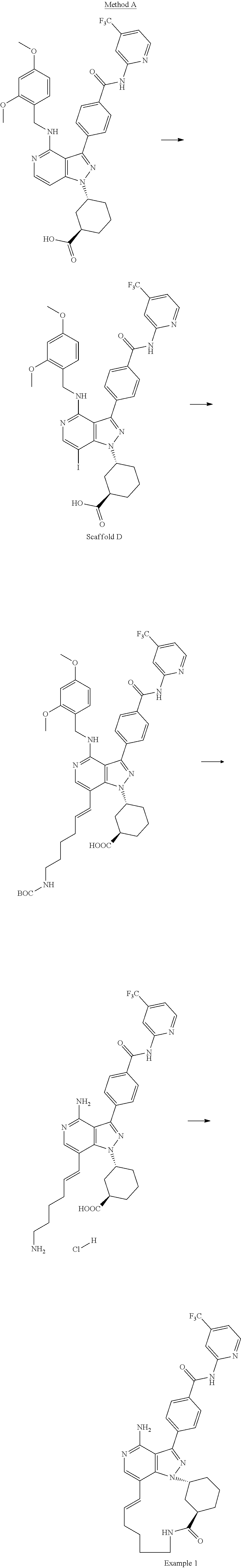

### Example 1

### (a) (1R,3R)-3-[4-[(2,4-dimethoxyphenyl)methylamino]-7-iodo-3-[4-[[4-(trifluoromethyl)-2-pyridyl]carbamoyl]phenyl]pyrazolo[4,3-c]pyridin-1-yl]cyclohexanecarboxylic acid (Scaffold D)

To a cold (0° C.) solution of (1R,3R)-3-[4-[(2,4-dimethoxyphenyl)methylamino]-3-[4-[[4-(trifluoromethyl)-2-pyridyl]carbamoyl]phenyl]pyrazolo[4,3-c]pyridin-1-yl]cyclohexanecarboxylic acid (Scaffold C) (1.72 g, 2.55 mmol) in DMF (13 mL) was added N-iodosuccinimide (544 mg, 2.42 mmol). The reaction mixture was stirred o/n allowing the mixture to warm up to room temperature. Still ˜6% of starting material remained present. Additional NIS (29 mg) was added to the reaction mixture and the mixture was stirred at room temperature o/n. The mixture was diluted with ethyl acetate and washed with sodium thiosulfate solution, water, brine, dried over sodium sulfate, filtered and concentrated in vacuo. The crude product was purified by column chromatography using SiO₂ and dichloromethane/methanol/acetic acid=99/1 to 95/5 v/v %. All fractions containing the title compound were collected and concentrated in vacuo to give 1.75 g of the title compound (Yield: 86%).

### (b) (1R,3R)-3-[7-[(E)-6-(tert-butoxycarbonylamino)hex-1-enyl]-4-[(2,4-dimethoxyphenyl)methyl-amino]-3-[4-[[4-(trifluoromethyl)-2-pyridyl]carbamoyl]phenyl]pyrazolo[4,3-cpyridin-1-yl]cyclo-hexanecarboxylic acid

A mixture of (1R,3R)-3-[7-[(E)-6-(tert-butoxycarbonylamino)hex-1-enyl]-4-[(2,4-dimethoxyphenyl)methyl-amino]-3-[4-[[4-(trifluoromethyl)-2-pyridyl]carbamoyl]phenyl]pyrazolo-[4,3-c]pyridin-1-yl]cyclo-hexanecarboxylic acid (100 mg, 0.125 mmol), tert-butyl N-[(E)-6-(4,4,5,5-tetramethyl-1,3,2-dioxaborolan-2-yl)hex-5-enyl]carbamate (Intermediate L1) (49 mg, 0.15 mmol) and cesium carbonate (122 mg, 0.375 mmol) in DMF/water=9/1 v/v % (1 mL) was degassed with nitrogen for 5 min at 30° C. CataCXium® A Pd G3 (4 mg, 0.006 mmol) was added and the mixture was again degassed with nitrogen for 5 min at 30° C. The reaction mixture was stirred at 65° C. o/n. After cooling ethyl acetate was added and the mixture was stirred for 5 min. The mixture was filtered over Decalite™ and the filtrate was washed with 5% citric acid solution, water and brine. The organic layer was separated, dried over sodium sulfate, filtered and then concentrated under reduced pressure. The residue was purified by column chromatography (dichloromethane/methanol=10/0 to 94/6 v/v %) to give 328 mg of the title compound (yield: 81.6%).

### (c) (1R,3R)-3-[4-Amino-7-[(E)-6-aminohex-1-enyl]-3-[4-[[4-(trifluoromethyl)-2-pyridyl]carba-moyl]phenyl]pyrazolo[4,3-c]pyridin-1-yl]cyclohexanecarboxylic acid hydrochloride

(1R,3R)-3-[7-[(E)-7-(tert-butoxycarbonylamino)hept-1-enyl]-4-[(2,4-dimethoxyphenyl)-methylamino]-3-(3-quinolyl)pyrazolo[4,3-c]pyridin-1-yl]cyclohexanecarboxylic acid (324 mg, 0.38 mmol) was dissolved in dichloromethane (3 mL). 4M HCl/dioxane solution (3 mL) was added and the mixture was stirred at room temperature o/n. The solvent was decantated and the residue triturated with dichloromethane, dried in vacuo, to give 340 mg the title compound in quantitative crude yield.

### (d) Example 1

(1R,3R)-3-[4-Amino-7-[(E)-6-aminohex-1-enyl]-3-[4-[[4-(trifluoromethyl)-2-pyridyl]car-bamoyl]phenyl]pyrazolo[4,3-c]pyridin-1-yl]cyclohexanecarboxylic acid hydrochloride (170 mg, 0.188 mmol) was dissolved in DMF (7.6 mL) and N-ethylmorpholine (119 μL, 0.94 mmol) was added. This solution was added at via a syringe pump at a rate of 0.7 rpm, to a stirred solution of HATU (214 mg, 0.564 mmol) and N-ethylmorpholine (72 μL, 0.564 mmol) in DMF (11.4 mL). Ethyl acetate (50 mL) was added to the reaction mixture and the mixture was washed with 5% NaHCO₃-solution and brine, dried over sodium sulfate, filtered and concentrated under reduced pressure. The residue was purified by column chromatography dichloromethane/methanol=10/0 to 94/6 v/v %). Purification was performed using preparative HPLC to afford the title compound (41 mg, 36%). Data: LCMS (B) Rₜ: 9.43 min; m/z 604.4 [M+H]⁺.

The following Examples were synthesized following method A described for Example 1.

| | | | | | | Found | | |
|---|---|---|---|---|---|---|---|---|
| Ex- | Meth- | Inter- | | Rₜ (min) | | Mass | | |
| ample | od | mediate | CHEMISTRY | (LCMS B) | Calc Mass | [M + H]⁺ | Amount | Yield |
| 1 | A | L1 | 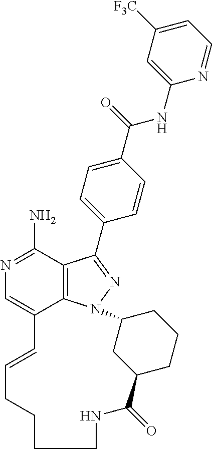 [SMILES: Nc1ncc2c3c1c(-c1ccc(C(=O)Nc4cc(C(F)(F)F)ccn4)cc1)nn3[C@@H]1CCC[C@H](C1)C(=O)NCCCC/C=C/2] | 9.43 | 603.25696 | 604.4 | 41 mg | 36% |
| 2 | A | L2 | 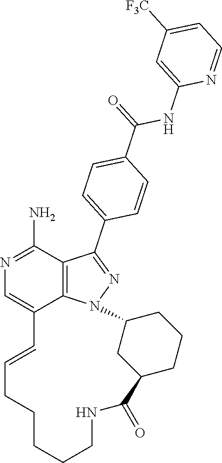 [SMILES: Nc1ncc2c3c1c(-c1ccc(C(=O)Nc4cc(C(F)(F)F)ccn4)cc1)nn3[C@@H]1CCC[C@H](C1)C(=O)NCCCCC/C=C/2] | 9.63 | 617.27261 | 618.4 | 70 mg | 31% |
| 3 | A | L3 | 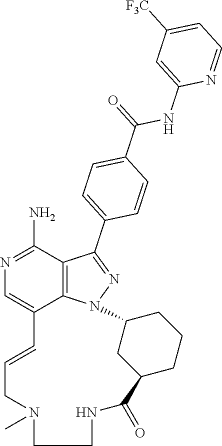 [SMILES: CN1C/C=C/c2cnc(N)c3c(-c4ccc(C(=O)Nc5cc(C(F)(F)F)ccn5)cc4)nn(c23)[C@@H]2CCC[C@H](C2)C(=O)NCC1] | 7.37 | 618.26786 | 619.4 | 41 mg | 63% |
| 4 | A | Commerc | 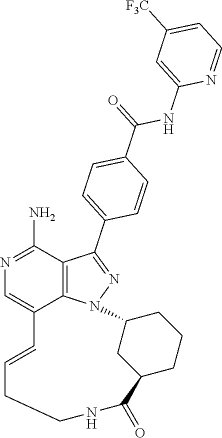 [SMILES: Nc1ncc2c3c1c(-c1ccc(C(=O)Nc4cc(C(F)(F)F)ccn4)cc1)nn3[C@@H]1CCC[C@H](C1)C(=O)NCC/C=C/2] | 8.76 | 575.22566 | 576.4 | 61 mg | 41% |
| 5 | A | L4 | 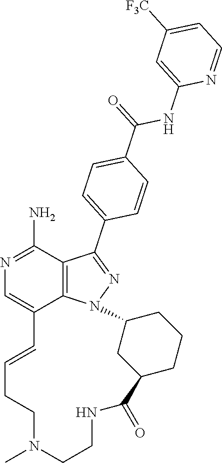 [SMILES: CN1CC/C=C/c2cnc(N)c3c(-c4ccc(C(=O)Nc5cc(C(F)(F)F)ccn5)cc4)nn(c23)[C@@H]2CCC[C@H](C2)C(=O)NCC1] | 7.15 | 632.28351 | 633.4 | 56 mg | 26% |

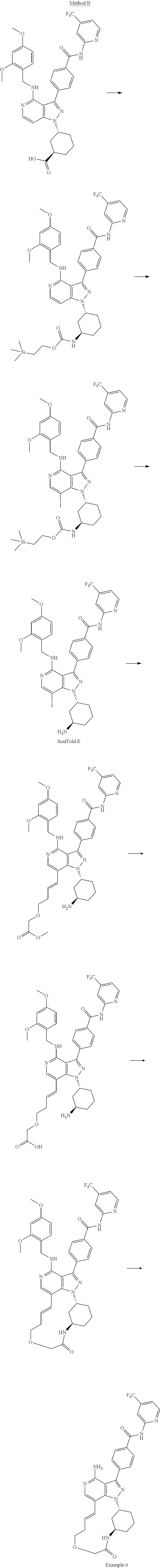

### Example 6

### (a) 2-Trimethylsilylethyl N-[(1R,3R)-3-[4-[(2,4-dimethoxyphenyl)methylamino]-3-[4-[[4-(trifluoro-methyl)-2-pyridyl]carbamoyl]phenyl]pyrazolo[4,3-c]pyridin-1-yl]cyclohexyl]carbamate

Diphenylphosphoryl azide (0.69 mL, 3.2 mmol) was added to a suspension of (1R,3R)— 3-[4-[(2,4-dimethoxyphenyl)methylamino]-3-[4-[[4-(trifluoromethyl)-2-pyridyl]carbamoyl]phenyl]-pyrazolo[4,3-c]pyridin-1-yl]cyclohexanecarboxylic acid (Scaffold C) (4.95 g, 7.30 mmol), triethylamine (1.44 mL, 10.3 mmol) in toluene (52 mL) and stirred for 30 min at 100° C. After cooling to room temperature, 2-(trimethylsilyl)ethanol (1.22 g, 10.3 mmol) was added and the reaction mixture was again stirred for 10 h. at 100° C. The reaction mixture was diluted with water (50 mL) and the mixture was stirred 30 minutes at room temperature. The layers were separated and the water layer was extracted with toluene (2×100 mL, slow settlement of layers). The combined organic layers were washed with water (100 mL), 1N NaOH (2×100 mL), water (100 mL) and concentrated and the residue was subjected to high vacuum to give 6.10 g 2-trimethylsilylethyl N-[(1R,3R)-3-[4-[(2,4-dimethoxyphenyl)methylamino]-3-[4-[[4-(trifluoromethyl)-2-pyridyl]carbamoyl]phenyl]-pyrazolo[4,3-c]pyridin-1-yl]cyclohexyl]carbamate as a light brown foam (yield>97%).

### (b) 2-Trimethylsilylethyl N-[(1R,3R)-3-[4-[(2,4-dimethoxyphenyl)methylamino]-7-iodo-3-[4-[[4-(trifluoromethyl)-2-pyridyl]carbamoyl]phenyl]pyrazolo[4,3-c]pyridin-1-yl]cyclohexyl]carbamate

To a cold (0° C.) solution of 2-trimethylsilylethyl N-[(1R,3R)-3-[4-[(2,4-dimethoxy-phenyl)methylamino]-3-[4-[[4-(trifluoromethyl)-2-pyridyl]carbamoyl]phenyl]-pyrazolo[4,3-c]pyridin-1-yl]cyclohexyl]carbamate (6.1 g, 7.7 mmol) in DMF (35 mL) was added N-iodosuccinimide (1.91 g, 8.5 mmol). The reaction mixture was stirred 2 h. allowing the mixture to warm up to room temperature. The mixture was diluted with ethyl acetate and washed with sodium thiosulfate solution, water, brine, dried over sodium sulfate, filtered and concentrated in vacuo. The crude product was purified by column chromatography using SiO₂ and heptane/ethyl acetate=95/5 to 6/4 v/v %. All fractions containing the title compound were collected and concentrated in vacuo to give 5.2 g of the title compound (Yield: 74%).

### (c) 4-[1-[(1R,3R)-3-aminocyclohexyl]-4-[(2,4-dimethoxyphenyl)methylamino]-7-iodo-pyrazolo-[4,3-c]pyridin-3-yl]-N-[4-(trifluoromethyl)-2-pyridyl]benzamide (Scaffold E)

2-Trimethylsilylethyl N-[(1R,3R)-3-[4-[(2,4-dimethoxyphenyl)methylamino]-7-iodo-3-[4-[[4-(trifluoromethyl)-2-pyridyl]carbamoyl]phenyl]pyrazolo[4,3-c]pyridin-1-yl]cyclohexyl]carbama-te (5.4 g, 5.90 mmol) was dissolved in MeCN (59 mL) to give a bright yellow solution.

Tetrabutylammonium fluoride, 1M solution in THE (6.5 mL; 6.5 mmol) was added and the resulting mixture was stirred at 60° C. for 18 h. The reaction mixture was added dropwise to a stirred 1/1-mixture, of sat. NaHCO₃-solution/ethyl acetate (400 mL). The resulting biphasic system was stirred 30 minutes at room temperature. The layers were separated and the water layer was extracted with ethyl acetate (50 mL). The combined organic layers were washed with 5% NaHCO₃-solution (3×200 mL), brine (75 mL), dried over sodium sulfate, filtered and concentrated to give 4.8 g of the title compound. (Yield: quantitative crude).

### (d) 2-[(E)-4-[1-[(1R,3R)-3-Aminocyclohexyl]-4-[(2,4-dimethoxyphenyl)methylamino]-3-[4-[[4-(trifluoromethyl)-2-pyridyl]carbamoyl]phenyl]pyrazolo[4,3-c]pyridin-7-yl]but-3-enoxylacetic acid

A mixture of 4-[1-[(1R,3R)-3-aminocyclohexyl]-4-[(2,4-dimethoxyphenyl)methylamino]-7-iodo-pyrazolo-[4,3-c]pyridin-3-yl]-N-[4-(trifluoromethyl)-2-pyridyl]benzamide (100 mg, 0.13 mmol), methyl 2-[(E)-4-(4,4,5,5-tetramethyl-1,3,2-dioxaborolan-2-yl)but-3-enoxy]acetate (Intermediate L5) (53 mg, 0.20 mmol) and potassium phosphate tribasic (83 mg, 0.39 mmol) in dioxane/water=4/1 v/v % (2 mL) was degassed with nitrogen for 5 min at 30° C. CataCXium® A Pd G3 (4.7 mg, 6.5 μmol) was added and the mixture was again degassed with nitrogen for 5 min at 30° C. The reaction mixture was stirred at 65° C. o/n. After cooling ethyl acetate was added and the mixture was stirred for 5 min. The mixture was filtered over Decalite™ and lyophilized to give 150 mg of the title compound (yield: quantitative crude).

### (e) Example 6

2-[(E)-4-[1-[(1R,3R)-3-Aminocyclohexyl]-4-[(2,4-dimethoxyphenyl)methylamino]-3-[4-[[4-(trifluoromethyl)-2-pyridyl]carbamoyl]phenyl]pyrazolo[4,3-c]pyridin-7-yl]but-3-enoxy]acetic acid (150 mg, 0.13 mmol) was added portion-wise to a stirred solution of HATU (148 mg, 0.39 mmol) and N-ethylmorpholine (83 μL, 0.65 mmol) in DMF (11.4 mL). and the reaction mixture was stirred for 4 h. at room temperature. Ethyl acetate (50 mL) was added to the reaction mixture and the mixture was washed with 5% NaHCO₃-solution and brine, dried over sodium sulfate, filtered and concentrated under reduced pressure. The residue was purified by column chromatography dichloromethane/ethyl acetate=10/0 to 0/10 v/v %) to give 30 mg of DMB-protected macrocycle Example 6. The product was dissolved in TFA/TIS/Water 90/5/5 v/v % (1 mL) and the mixture was stirred at room temperature for 2 h. The mixture was concentrated and the residue was co-evaporated with DCM (2 mL). The residue was dissolved in DCM/MeOH=9/1 v/v % (3 mL) and 5% aq. NaHCO₃-solution (4 mL) was added (pH>8). The layers were separated and the water layer was extracted with DCM/MeOH=9/1 v/v % (2×3 mL). The combined organic layers were filtered over a PE-filter and concentrated under reduced pressure. Purification was performed using preparative HPLC to afford the title compound (7 mg). Data: LCMS (B) Rₜ: 9.17 min; m/z 606.4 [M+H]⁺.

The following Examples were synthesized following method B described for Example 6.

| | | | | | | Found | | | |
|---|---|---|---|---|---|---|---|---|---|
| Ex- | Meth- | Inter- | | Rₜ (min) | | Mass | | | |
| ample | od | mediate | CHEMISTRY | (LCMS B) | Calc Mass | [M + H]⁺ | Amount | | Yield |
| 6 | B | L5 | 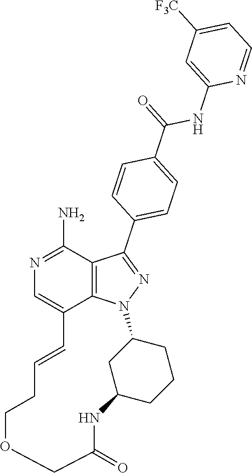 [SMILES: Nc1ncc2c3c1c(-c1ccc(C(=O)Nc4cc(C(F)(F)F)ccn4)cc1)nn3[C@@H]1CCC[C@H](C1)NC(=O)COCC/C=C/2] | 9.17 | 605.23622 | 606.4 | 7 | mg | |
| 7 | B | L6 | 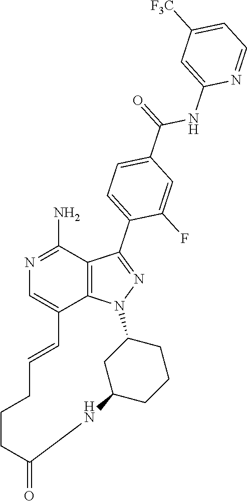 [SMILES: Nc1ncc2c3c1c(-c1ccc(C(=O)Nc4cc(C(F)(F)F)ccn4)cc1F)nn3[C@@H]1CCC[C@H](C1)NC(=O)CCC/C=C/2] | 9.33 | 607.23189 | 608.5 | 21 | mg | 25% |
| 8 | B | L7 | 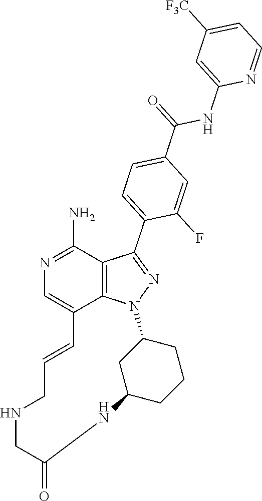 [SMILES: Nc1ncc2c3c1c(-c1ccc(C(=O)Nc4cc(C(F)(F)F)ccn4)cc1F)nn3[C@@H]1CCC[C@H](C1)NC(=O)CNC/C=C/2] | 7.25 | 608.22714 | 609.4 | 23 | mg | 66% |
| 9 | B | L8 | 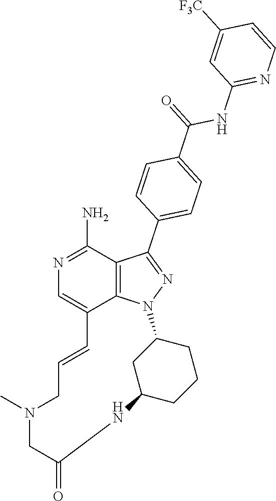 [SMILES: CN1C/C=C/c2cnc(N)c3c(-c4ccc(C(=O)Nc5cc(C(F)(F)F)ccn5)cc4)nn(c23)[C@@H]2CCC[C@H](C2)NC(=O)C1] | 6.70 | 604.25221 | 605.5 | 7.5 | mg | 31% |
| 10 | B | L9 | 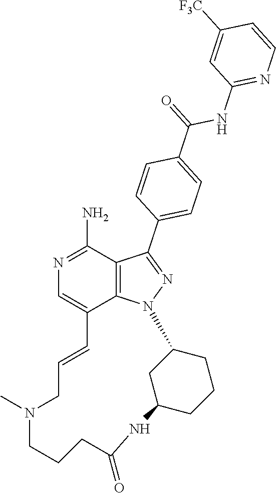 [SMILES: CN1C/C=C/c2cnc(N)c3c(-c4ccc(C(=O)Nc5cc(C(F)(F)F)ccn5)cc4)nn(c23)[C@@H]2CCC[C@H](C2)NC(=O)CCC1] | 6.82 | 632.28351 | 633.5 | 8.8 | mg | 40% |
| 11 | B | L10 | 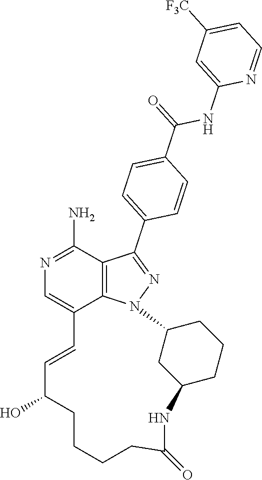 [SMILES: Nc1ncc2c3c1c(-c1ccc(C(=O)Nc4cc(C(F)(F)F)ccn4)cc1)nn3[C@@H]1CCC[C@H](C1)NC(=O)CCCC[C@H](O)/C=C/2] | 8.40 | 633.26752 | 634.4 | 22.8 | mg | 32.4% |
| 12 | B | L11 | 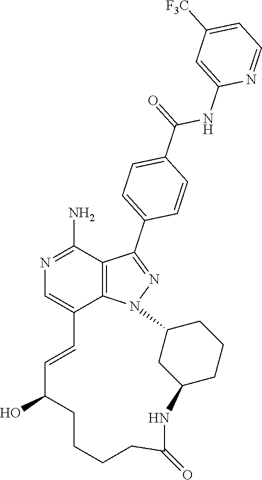 [SMILES: Nc1ncc2c3c1c(-c1ccc(C(=O)Nc4cc(C(F)(F)F)ccn4)cc1)nn3[C@@H]1CCC[C@H](C1)NC(=O)CCCC[C@@H](O)/C=C/2] | 8.27 | 633.26752 | 634.4 | 20.9 | mg | 22.5% |
| 13 | B | L12 | 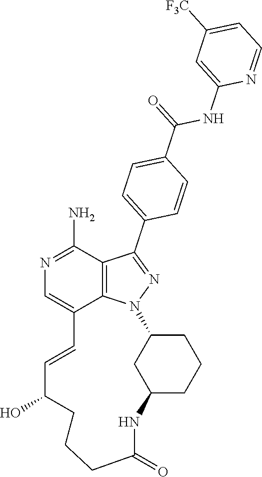 [SMILES: Nc1ncc2c3c1c(-c1ccc(C(=O)Nc4cc(C(F)(F)F)ccn4)cc1)nn3[C@@H]1CCC[C@H](C1)NC(=O)CCC[C@H](O)/C=C/2] | 8.38 | 619.25187 | 620.4 | 20.5 | mg | 22.9% |
| 14 | B | L13 | 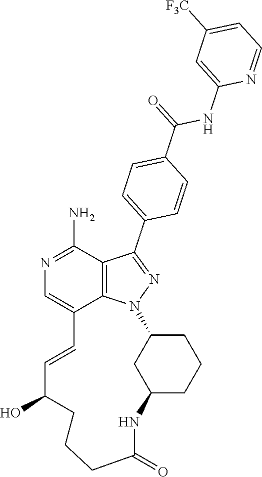 [SMILES: Nc1ncc2c3c1c(-c1ccc(C(=O)Nc4cc(C(F)(F)F)ccn4)cc1)nn3[C@@H]1CCC[C@H](C1)NC(=O)CCC[C@@H](O)/C=C/2] | 8.27 | 619.25187 | 620.4 | 19.2 | mg | 29% |
| 15 | B | L14 | 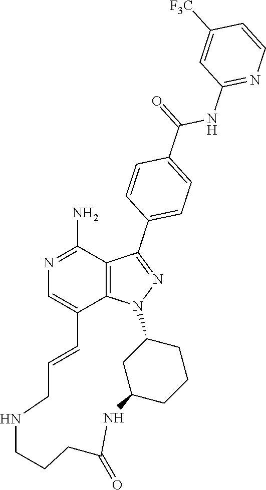 [SMILES: Nc1ncc2c3c1c(-c1ccc(C(=O)Nc4cc(C(F)(F)F)ccn4)cc1)nn3[C@@H]1CCC[C@H](C1)NC(=O)CCCNC/C=C/2] | 7.44 | 618.26786 | 619.4 | 22.4 | mg | 64% |
| 16 | B | L16 | 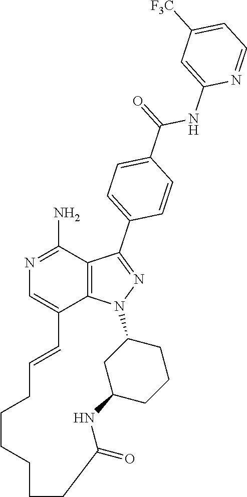 [SMILES: Nc1ncc2c3c1c(-c1ccc(C(=O)Nc4cc(C(F)(F)F)ccn4)cc1)nn3[C@@H]1CCC[C@H](C1)NC(=O)CCCCCC/C=C/2] | 9.53 | 631.28826 | 632.4 | 9 | mg | 39% |
| 17 | B | L17 | 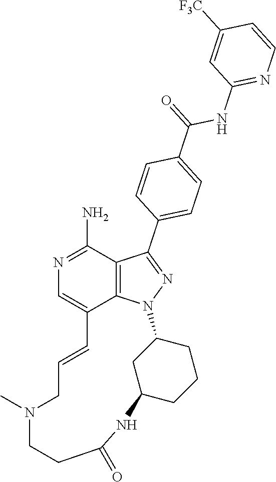 [SMILES: CN1C/C=C/c2cnc(N)c3c(-c4ccc(C(=O)Nc5cc(C(F)(F)F)ccn5)cc4)nn(c23)[C@@H]2CCC[C@H](C2)NC(=O)CC1] | 7.18 | 618.26786 | 619.4 | 10 | mg | 51.3% |
| 18 | B | L18 | 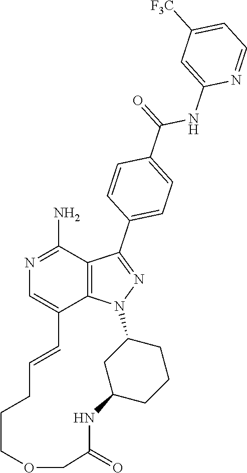 [SMILES: Nc1ncc2c3c1c(-c1ccc(C(=O)Nc4cc(C(F)(F)F)ccn4)cc1)nn3[C@@H]1CCC[C@H](C1)NC(=O)COCCC/C=C/2] | 9.28 | 619.25187 | 620.4 | 7 | mg | 28.4% |
| 19 | B | L12 | 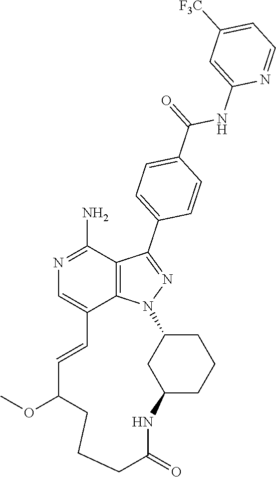 [SMILES: COC1/C=C/c2cnc(N)c3c(-c4ccc(C(=O)Nc5cc(C(F)(F)F)ccn5)cc4)nn(c23)[C@@H]2CCC[C@H](C2)NC(=O)CCC1] | 4.07 (LCMS (A) | 633.26752 | 634.4 | 6.8 | mg | 14.1% |
| 20 | B | L19 | 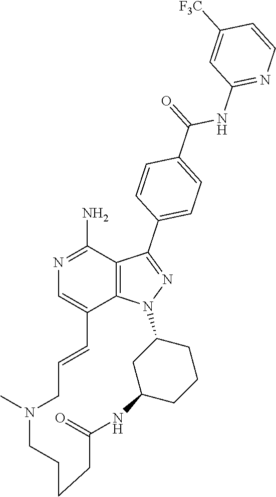 [SMILES: CN1C/C=C/c2cnc(N)c3c(-c4ccc(C(=O)Nc5cc(C(F)(F)F)ccn5)cc4)nn(c23)[C@@H]2CCC[C@H](C2)NC(=O)CCCC1] | 7.10 | 646.29916 | 647.4 | 12.7 | mg | 29.4% |
| 21 | B | L20 | 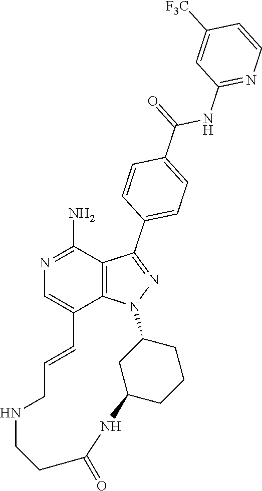 [SMILES: Nc1ncc2c3c1c(-c1ccc(C(=O)Nc4cc(C(F)(F)F)ccn4)cc1)nn3[C@@H]1CCC[C@H](C1)NC(=O)CCNC/C=C/2] | 7.12 | 604.25221 | 605.3 | 16.8 | mg | 49.2% |
| 22 | B | L21 | 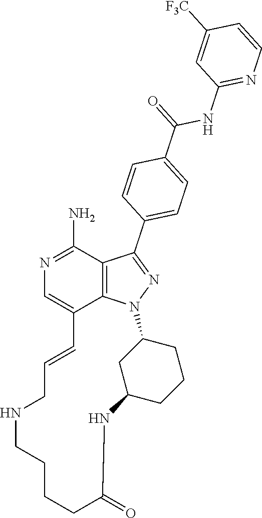 [SMILES: Nc1ncc2c3c1c(-c1ccc(C(=O)Nc4cc(C(F)(F)F)ccn4)cc1)nn3[C@@H]1CCC[C@H](C1)NC(=O)CCCCNC/C=C/2] | 9.09 | 632.28351 | 633.4 | 32.1 | mg | 78.4% |
| 23 | B | L22 | 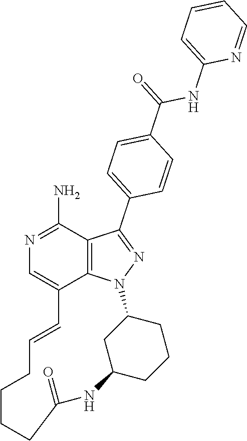 [SMILES: Nc1ncc2c3c1c(-c1ccc(C(=O)Nc4ccccn4)cc1)nn3[C@@H]1CCC[C@H](C1)NC(=O)CCCC/C=C/2] | 6.50 | 535.26957 | 536.4 | 25 | mg | 31% |
| 24 | B | L15 | 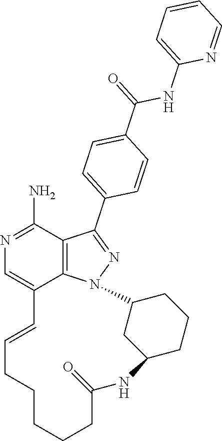 | 6.79 | 549.28522 | 550.4 | 33 | mg | 46% |
| 25 | B | L23 | 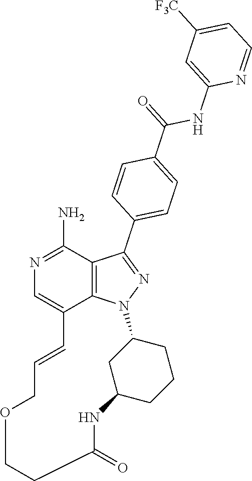 [SMILES: Nc1ncc2c3c1c(-c1ccc(C(=O)Nc4cc(C(F)(F)F)ccn4)cc1)nn3[C@@H]1CCC[C@H](C1)NC(=O)CCOC/C=C/2] | 8.87 | 605.23622 | 606.3 | 7 | mg | 28% |
| 26 | B | L24 | 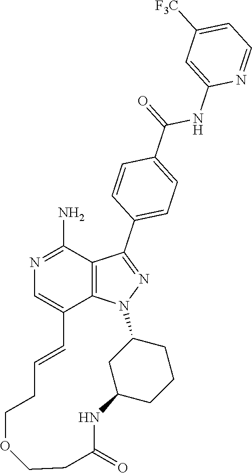 [SMILES: Nc1ncc2c3c1c(-c1ccc(C(=O)Nc4cc(C(F)(F)F)ccn4)cc1)nn3[C@@H]1CCC[C@H](C1)NC(=O)CCOCC/C=C/2] | 8.98 | 619.25187 | 620.4 | 7 | mg | 37.3% |
| 27 | B | L25 | 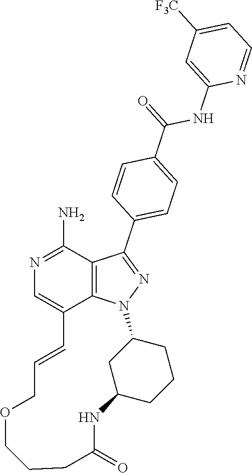 [SMILES: Nc1ncc2c3c1c(-c1ccc(C(=O)Nc4cc(C(F)(F)F)ccn4)cc1)nn3[C@@H]1CCC[C@H](C1)NC(=O)CCCOC/C=C/2] | 9.07 | 619.25187 | 620.4 | 7 | mg | 37% |
| 28 | B | L26 | 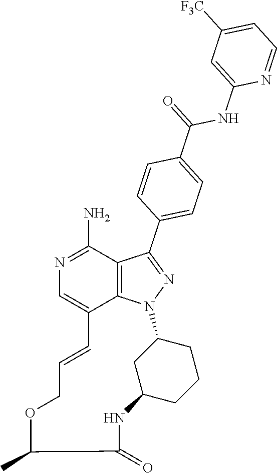 [SMILES: C[C@H]1OC/C=C/c2cnc(N)c3c(-c4ccc(C(=O)Nc5cc(C(F)(F)F)ccn5)cc4)nn(c23)[C@@H]2CCC[C@H](C2)NC1=O] | 9.14 | 605.23622 | 606.4 | 6 | mg | 22.7% |
| 29 | B | L16 | 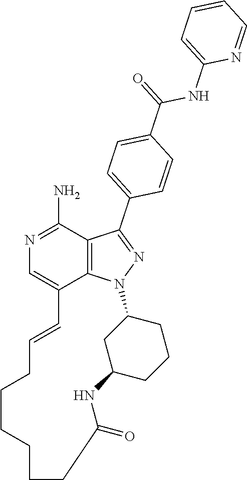 [SMILES: Nc1ncc2c3c1c(-c1ccc(C(=O)Nc4ccccn4)cc1)nn3[C@@H]1CCC[C@H](C1)NC(=O)CCCCCC/C=C/2] | 7.29 | 563.30087 | 564.4 | 11.8 | mg | 65% |
| 30 | B | L27 | 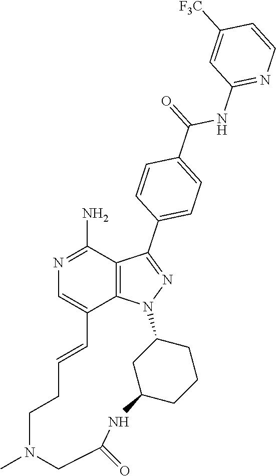 [SMILES: CN1CC/C=C/c2cnc(N)c3c(-c4ccc(C(=O)Nc5cc(C(F)(F)F)ccn5)cc4)nn(c23)[C@@H]2CCC[C@H](C2)NC(=O)C1] | 6.88 | 618.26786 | 619.4 | 7.2 | mg | 40.6% |
| 31 | B | L28 | 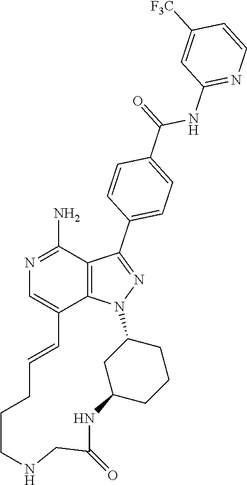 [SMILES: Nc1ncc2c3c1c(-c1ccc(C(=O)Nc4cc(C(F)(F)F)ccn4)cc1)nn3[C@@H]1CCC[C@H](C1)NC(=O)CNCCC/C=C/2] | 6.88 | 618.26786 | 619.4 | 6 | mg | 30% |
| 32 | B | L29 | 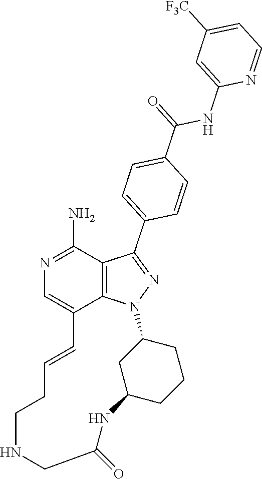 [SMILES: Nc1ncc2c3c1c(-c1ccc(C(=O)Nc4cc(C(F)(F)F)ccn4)cc1)nn3[C@@H]1CCC[C@H](C1)NC(=O)CNCC/C=C/2] | 6.99 | 604.25221 | 605.4 | 32 | mg | 76% |
| 33 | B | L30 | 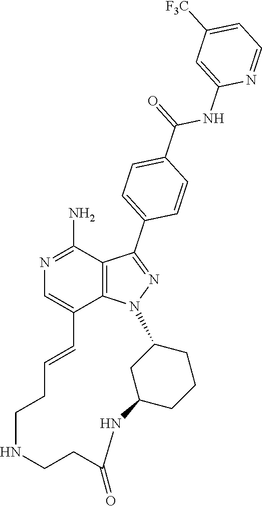 [SMILES: Nc1ncc2c3c1c(-c1ccc(C(=O)Nc4cc(C(F)(F)F)ccn4)cc1)nn3[C@@H]1CCC[C@H](C1)NC(=O)CCNCC/C=C/2] | 6.99 | 618.26786 | 619.4 | 39 | mg | 48% |
| 34 | B | L31 | 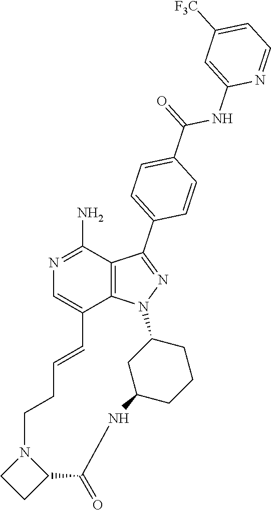 | 7.19 | 630.26786 | 631.4 | 24 | mg | 54% |
| 35 | B | L29 | 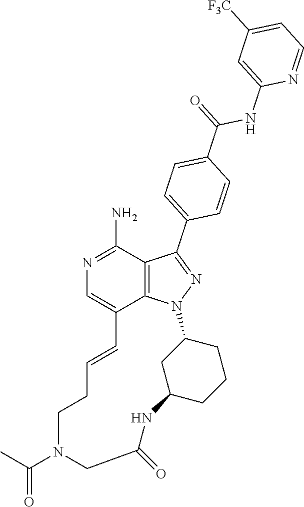 [SMILES: CC(=O)N1CC/C=C/c2cnc(N)c3c(-c4ccc(C(=O)Nc5cc(C(F)(F)F)ccn5)cc4)nn(c23)[C@@H]2CCC[C@H](C2)NC(=O)C1] | 8.78 | 646.26277 | 647.4 | 7.5 | mg | 72% |
| 36 | B | L30 | 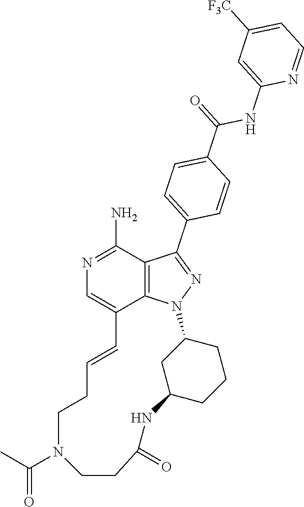 [SMILES: CC(=O)N1CC/C=C/c2cnc(N)c3c(-c4ccc(C(=O)Nc5cc(C(F)(F)F)ccn5)cc4)nn(c23)[C@@H]2CCC[C@H](C2)NC(=O)CC1] | 8.78 | 660.27842 | 661.4 | 11 | mg | 56% |
| 37 | B | L15 | 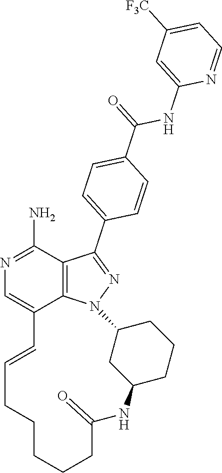 [SMILES: Nc1ncc2c3c1c(-c1ccc(C(=O)Nc4cc(C(F)(F)F)ccn4)cc1)nn3[C@@H]1CCC[C@H](C1)NC(=O)CCCCC/C=C/2] | 9.29 | 617.27261 | 618.4 | 14 | mg | 25% |
| 38 | B | L32 | 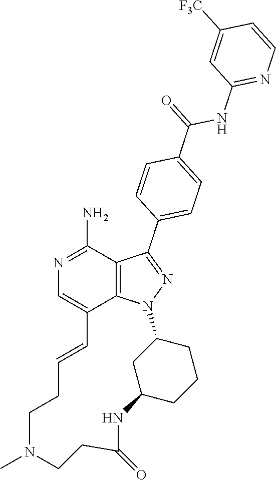 [SMILES: CN1CC/C=C/c2cnc(N)c3c(-c4ccc(C(=O)Nc5cc(C(F)(F)F)ccn5)cc4)nn(c23)[C@@H]2CCC[C@H](C2)NC(=O)CC1] | 7.01 | 632.28351 | 633.4 | 22 | mg | 42.6% |
| 39 | B | L33 | 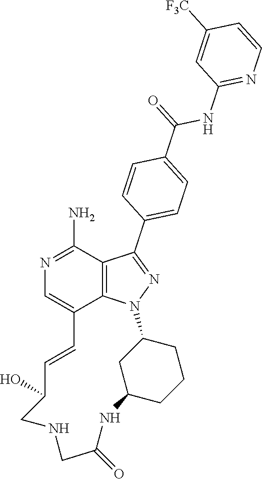 [SMILES: Nc1ncc2c3c1c(-c1ccc(C(=O)Nc4cc(C(F)(F)F)ccn4)cc1)nn3[C@@H]1CCC[C@H](C1)NC(=O)CNC[C@@H](O)/C=C/2] | 7.03 | 620.24712 | 621.3 | 7.5 | mg | 24.4% |
| 40 | B | L34 | 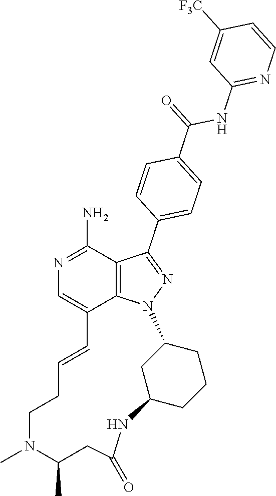 [SMILES: C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCN1C] | 7.08 | 646.29916 | 647.5 | 36 | mg | 44.9% |
| 41 | B | L35 | 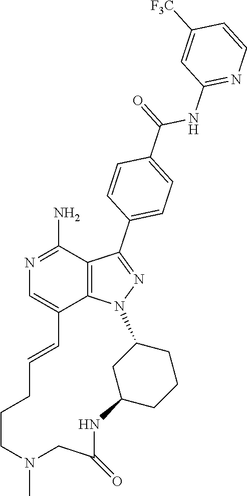 [SMILES: CN1CCC/C=C/c2cnc(N)c3c(-c4ccc(C(=O)Nc5cc(C(F)(F)F)ccn5)cc4)nn(c23)[C@@H]2CCC[C@H](C2)NC(=O)C1] | 6.76 | 632.28351 | 633.4 | 10 | mg | 63% |
| 42 | B | L36 | 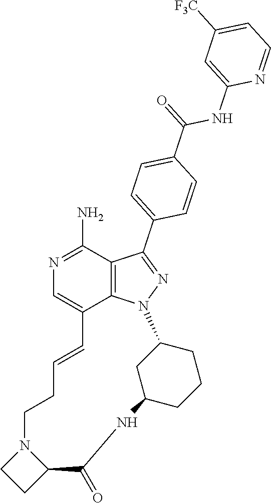 [SMILES: Nc1ncc2c3c1c(-c1ccc(C(=O)Nc4cc(C(F)(F)F)ccn4)cc1)nn3[C@@H]1CCC[C@H](C1)NC(=O)[C@H]1CCN1CC/C=C/2] | 8.40 | 630.26786 | 631.4 | 11 | mg | 29% |
| 43 | B | L37 | 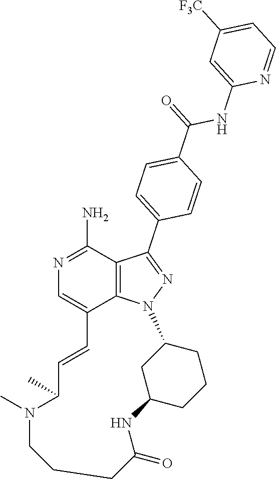 [SMILES: C[C@H]1/C=C/c2cnc(N)c3c(-c4ccc(C(=O)Nc5cc(C(F)(F)F)ccn5)cc4)nn(c23)[C@@H]2CCC[C@H](C2)NC(=O)CCCN1C] | 7.05 | 646.29916 | 647.5 | 5 | mg | 59% |
| 44 | B | L38 | 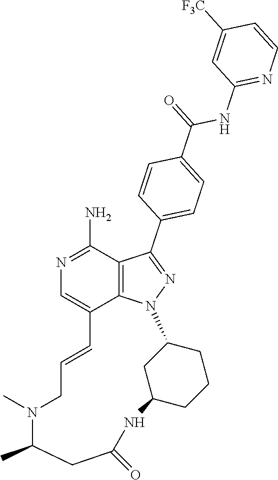 [SMILES: C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CN1C] | 7.12 | 632.28351 | 633.4 | 21.6 | mg | 89% |
| 45 | B | L39 | 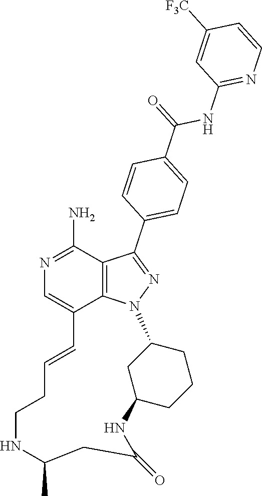 [SMILES: C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCN1] | 7.00 | 632.28351 | 633.5 | 24 | mg | 46.5% |

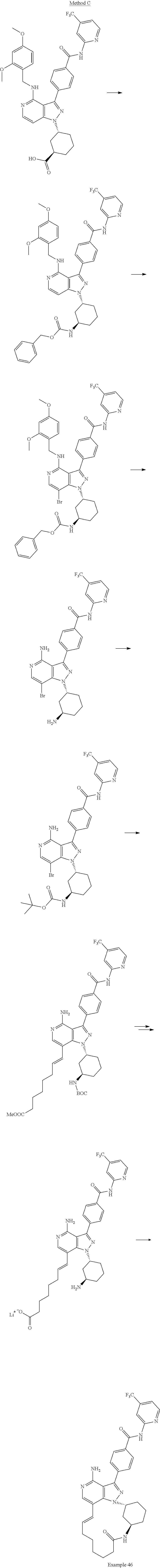

### Example 46

### (a) Benzyl N-[(1R,3R)-3-[4-[(2,4-dimethoxyphenyl)methylamino]-3-[4-[[4-(trifluoromethyl)-2-pyridyl]carbamoyl]phenyl]pyrazolo[4,3-c]pyridin-1-yl]cyclohexyl]carbamate

Diphenylphosphoryl azide (0.39 mL, 1.43 mmol) was added to a suspension of (1R,3R)— 3-[4-[(2,4-dimethoxyphenyl)methylamino]-3-[4-[[4-(trifluoromethyl)-2-pyridyl]carba-moyl]phenyl]-pyrazolo[4,3-c]pyridin-1-yl]cyclohexanecarboxylic acid (Scaffold C) (963 mg, 1.43 mmol), triethylamine (0.219 mL, 1.57 mmol) in toluene (15 mL) and stirred for 1 h. at 100° C.

After cooling to room temperature, benzyl alcohol (0.74 mL, 7.15 mmol) was added and the reaction mixture was again stirred for 2 h. at 80° C. The reaction mixture was diluted with DCM and washed with 5% citric acid solution, 5% aq. NaHCO₃-solution, brine, dried over sodium sulfate, filtered and concentrated under reduced pressure. The crude product was purified by column chromatography using SiO₂ and heptane/ethyl acetate=1/1 to 1/4 v/v %. All fractions containing the title compound were collected and concentrated in vacuo to give 652 mg of the title compound (Yield: 58%).

### (b) Benzyl N-[(1R,3R)-3-[7-bromo-4-[(2,4-dimethoxyphenyl)methylamino]-3-[4-[[4-(trifluoro-methyl)-2-pyridyl]carbamoyl]phenyl]pyrazolo[4,3-c]pyridin-1-yl]cyclohexyl]carbamate

To a cold (0° C.) solution of benzyl N-[(1R)-3-[(1R)-4-[(2,4-dimethoxyphenyl)-methylamino]-3-[4-[[4-(trifluoromethyl)-2-pyridyl]carbamoyl]phenyl]pyrazolo[4,3-c]pyridin-1-yl]cyclohexyl]carbamate (932 mg, 1.2 mmol) in acetonitrile (35 mL) was added N-bromosuccinimide (2×202 mg, 1.14 mmol). The reaction mixture was stirred 2 h. allowing the mixture to warm up to room temperature. The mixture was diluted with ethyl acetate and washed with sodium thiosulfate solution, water, brine, dried over sodium sulfate, filtered and concentrated in vacuo. The crude product was purified by column chromatography using SiO₂ and heptane/ethyl acetate=95/5 to 6/4 v/v %. All fractions containing the title compound were collected and concentrated in vacuo to give 856 mg of the title compound (Yield: 83%).

### (c) 4-[4-Amino--[(1R,3R)-3-aminocyclohexyl]-7-bromo-pyrazolo[4,3-c]pyridin-3-yl]-N-[4-(trifluoromethyl)-2-pyridyl]benzamide

Benzyl N-[(1R,3R)-3-[7-bromo-4-[(2,4-dimethoxyphenyl)methylamino]-3-[4-[[4-(trifluo-romethyl)-2-pyridyl]carbamoyl]phenyl]pyrazolo[4,3-c]pyridin-1-yl]cyclohexyl]carbamate (855 mg 1 mmol) was dissolved in TFA/H₂O=9/1 v/v % (10 mL). The reaction mixture was stirred for 5 h. at 60° C. The mixture was concentrated under reduced pressure and co-evaporated with toluene (3×25 mL). Dark purple oil was dissolved in DCM (50 mL) and water (50 mL) was added. After stirring for 15 min at room temperature, the water layer was separated. Water layer basified using 2N NaOH-solution, stirred for 15 min. and extracted with DCM/sec-BuOH=3/2 v/v % (2×50 mL). The combined organic layers was separated over an PE-filter and concentrated under reduced pressure to give 490 mg of the title compound (Yield: 85%).

### (d) tert-Butyl N-[(1R,3R)-3-[4-amino-7-bromo-3-[4-[[4-(trifluoromethyl)-2-pyridyl]carbamoyl]-phenyl]pyrazolo[4.3-c]pyridin-1-yl]cyclohexyl]carbamate

To a suspension of 4-[4-amino-1-[(1R,3R)-3-aminocyclohexyl]-7-bromo-pyrazolo[4,3-c]pyridin-3-yl]-N-[4-(trifluoromethyl)-2-pyridyl]benzamide (97 mg, 0.17 mmol) in DCM (5 mL) was added subsequently Et₃N (47 μl, 0.34 mmol) and a solution of BOC₂O (41 mg, 0.19 mmol) in DCM (2 mL). The reaction mixture was stirred for 2 h. at room temperature. DCM (10 mL) was added to the mixture and the organic phase was washed with water and brine. The DCM-layer was separated by filtration over an PE-filter and concentrated under reduced pressure to give 110 mg (96%) of the title compound.

### (e) Methyl(E)-8-[4-amino--[(1R,3R)-3-(tert-butoxycarbonylamino)cyclohexyl]-3-[4-[[4-(trifluoro-methyl)-2-pyridyl]carbamoyl]phenyl]pyrazolo[4,3-c]pyridin-7-yl]oct-7-enoate

A mixture of tert-butyl N-[(1R,3R)-3-[4-amino-7-bromo-3-[4-[[4-(trifluoromethyl)-2-pyri-dyl]carbamoyl]-phenyl]pyrazolo[4,3-c]pyridin-1-yl]cyclohexyl]carbamate (75 mg, 0.11 mmol), methyl(E)-8-(4,4,5,5-tetramethyl-1,3,2-dioxaborolan-2-yl)oct-7-enoate (Intermediate L15) (41 mg, 0.14 mmol) and potassium phosphate tribasic (70 mg, 0.33 mmol) in dioxane/water=4/1 v/v % (1.3 mL) was degassed with nitrogen for 5 min at 30° C. CataCXium® A Pd G3 (4 mg, 0.006 mmol) was added and the mixture was again degassed with nitrogen for 5 min at 30° C. The reaction mixture was stirred for 1 h at 100° C. under microwave radiation. After cooling ethyl acetate was added and the mixture was stirred for 5 min. The mixture was filtered over Decalite™ and the filtrate was washed with 5% citric acid solution, water and brine. The organic layer was separated, dried over sodium sulfate, filtered and then concentrated under reduced pressure. The residue was purified by column chromatography (dichloromethane/methanol=10/0 to 9/1 v/v %) to give 76 mg of the title compound (yield: 92%).

### (f) Lithium (E)-8-[4-amino--[(1R,3R)-3-aminocyclohexyl]-3-[4-[[4-(trifluoromethyl)-2-pyridyl]car-bamoyl]phenyl]pyrazolo[4,3-c]pyridin-7-yl]oct-7-enoate

To a solution of methyl(E)-8-[4-amino-1-[(1R,3R)-3-(tert-butoxycarbonylamino) cyclohexyl]-3-[4-[[4-(trifluoromethyl)-2-pyridyl]carbamoyl]phenyl]pyrazolo[4,3-c]pyridin-7-yl]oct-7-enoate (76 mg 0.1 mmol) in dioxane (557 μL) was added 4N HCl/dioxane (557 μL). The reaction mixture was stirred for 2 h. at room temperature. The mixture was concentrated under reduced pressure and the residual oil dissolved in DCM (50 mL) and water (50 mL) was added. After stirring for 15 min at room temperature, the water layer was separated. Water layer basified using 2N NaOH-solution, stirred for 15 min. and extracted with DCM/sec-BuOH=3/2 v/v % (2×50 mL). The combined organic layers were separated over an PE-filter and concentrated under reduced pressure. The crude product was dissolved in dioxane (1 mL) and subsequently water (162 μL) and 0.5N LiOH-solution (162 μL) were added. The mixture was stirred at room temperature for 4.5 h. The mixture was lyophilized to give 55 mg of the title compound (yield: quantitative crude).

### (g) Example 46

Lithium (E)-8-[4-amino-1-[(1R,3R)-3-aminocyclohexyl]-3-[4-[[4-(trifluoromethyl)-2-pyri-dyl]carbamoyl]phenyl]pyrazolo[4,3-c]pyridin-7-yl]oct-7-enoate (55 mg, 0.08 mmol) was dissolved in DMF (3.1 mL) and N-ethylmorpholine (20 μL, 0.15 mmol) was added. This solution was added at batchwise (20×155 μL every 5 mi), to a stirred solution of HATU (88 mg, 0.23 mmol) and N-ethylmorpholine (29.4 μL, 0.23) in DMF (4.6 mL). The mixture was stirred at room temperature for 30 mi after addition was complete. Ethyl acetate (50 mL) was added to the reaction mixture and the mixture was washed with 5% NaHCO₃-solution and brine, dried over sodium sulfate, filtered and concentrated under reduced pressure. The residue was purified by column chromatography dichloromethane/methanol=10/0 to 9/1 v/v %). Purification was performed using preparative HPLC to afford the title compound (21 mg, 44%). Data: LMS (B) Rₜ: 9.05 min; m/z 618.5 [M+H]⁺.

The following Examples were synthesized following method C described for Example 46.

| | | | | | | Found | | | |
|---|---|---|---|---|---|---|---|---|---|
| Ex- | | Inter- | | Rₜ (min) | | Mass | | | |
| ample | Method | mediate | CHEMISTRY | (LCMS B) | Calc Mass | [M + H]⁺ | Amount | | Yield |
| 46 | C | L15 | 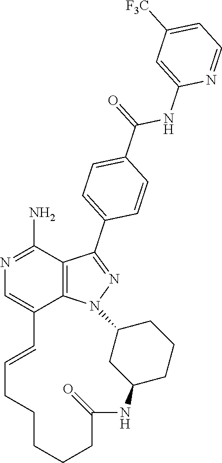 [SMILES: Nc1ncc2c3c1c(-c1ccc(C(=O)Nc4cc(C(F)(F)F)ccn4)cc1)nn3[C@@H]1CCC[C@H](C1)NC(=O)CCCCC/C=C/2] | 9.05 | 617.27261 | 618.5 | 21 | mg | 44% |
| 47 | C | L6 | 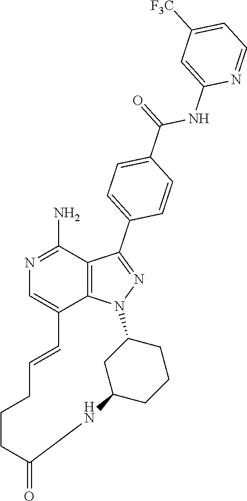 [SMILES: Nc1ncc2c3c1c(-c1ccc(C(=O)Nc4cc(C(F)(F)F)ccn4)cc1)nn3[C@@H]1CCC[C@H](C1)NC(=O)CCC/C=C/2] | 9.04 | 589.24131 | 590.4 | 33.8 | mg | 41% |
| 48 | C | L22 | 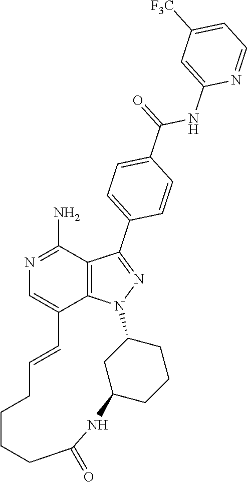 [SMILES: Nc1ncc2c3c1c(-c1ccc(C(=O)Nc4cc(C(F)(F)F)ccn4)cc1)nn3[C@@H]1CCC[C@H](C1)NC(=O)CCCC/C=C/2] | 9.15 | 603.25696 | 604.4 | 35.3 | mg | 50% |
| 49 | C | L40 | 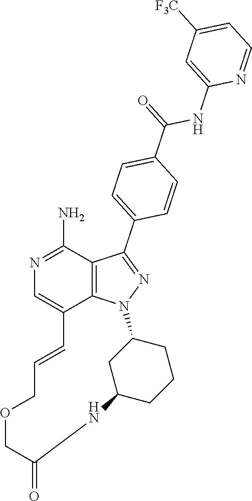 [SMILES: Nc1ncc2c3c1c(-c1ccc(C(=O)Nc4cc(C(F)(F)F)ccn4)cc1)nn3[C@@H]1CCC[C@H](C1)NC(=O)COC/C=C/2] | 3.75 (LCMS A) | 591.22057 | 592.3 | 5.6 | mg | 14% |

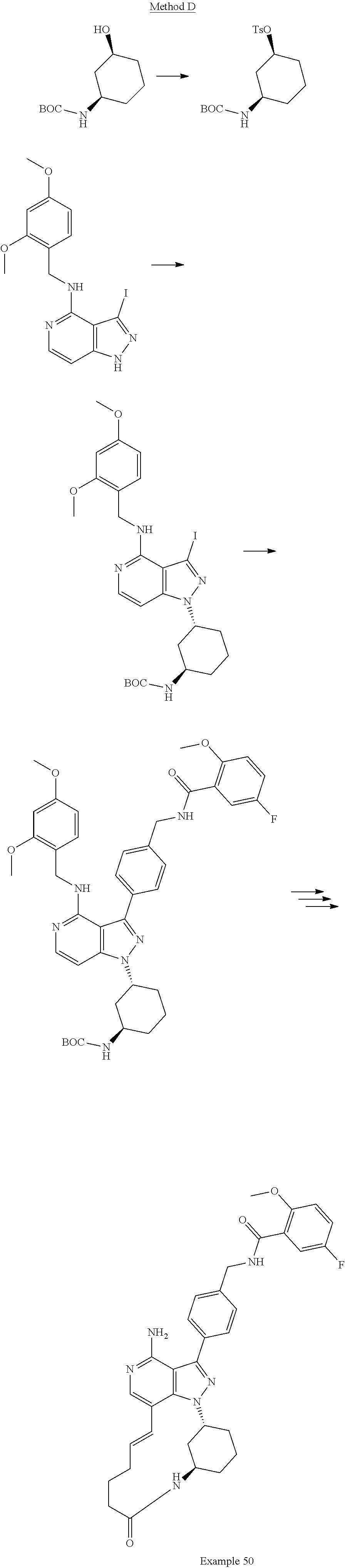 [SMILES: CC(C)(C)OC(=O)N[C@@H]1CCC[C@H](O)C1 ; Cc1ccc(S(=O)(=O)O[C@H]2CCC[C@@H](NC(=O)OC(C)(C)C)C2)cc1 ; COc1ccc(CNc2nccc3[nH]nc(I)c23)c(OC)c1 ; COc1ccc(CNc2nccc3c2c(I)nn3[C@@H]2CCC[C@@H](NC(=O)OC(C)(C)C)C2)c(OC)c1 ; COc1ccc(CNc2nccc3c2c(-c2ccc(CNC(=O)c4cc(F)ccc4OC)cc2)nn3[C@@H]2CCC[C@@H](NC(=O)OC(C)(C)C)C2)c(OC)c1 ; COc1ccc(F)cc1C(=O)NCc1ccc(-c2nn3c4c(cnc(N)c24)/C=C/CCCC(=O)N[C@@H]2CCC[C@@H]3C2)cc1]

### Example 50

### (a) [(1 S,3R)-3-(tert-butoxycarbonylamino)cyclohexyl]4-methylbenzenesulfonate

To a solution of tert-butyl N-[(1R,3S)-3-hydroxycyclohexyl]carbamate (Intermediate RP4) (1 g, 4.64 mmol) in dichloromethane (10 mL) was added triethylamine (712 μL, 5.11 mmol) and 4-DMAP (57 mg, 0.46 mmol) and the mixture was stirred for 5 min. p-toluenesulfonyl chloride (5.15 g, 27.0 mmol) was added portion-wise over a period of 4 h. and the reaction mixture was stirred at room temperature for 7 days. The mixture was washed with 5% citric acid solution, filtered over a PE-filter and concentrated under reduced pressure, the resulting residue was purified by column chromatography (heptane/ethyl acetate=8/2 v/v %) to give 1.3 g of the title compound (Yield: 75.8%).

### (b) tert-butyl N-[(1R,3R)-3-[4-[(2,4-dimethoxyphenyl)methylamino]-3-iodo-pyrazolo[4,3-c]pyri-din-1-yl]cyclohexyl]carbamate

To a solution of N-[(2,4-dimethoxyphenyl)methyl]-3-iodo-1 H-pyrazolo[4,3-c]pyridin-4-amine (Scaffold A) (1.62 g, 3.95 mmol) in DMF (20 mL) was added cesium carbonate (1.93 g, 5.93 mmol) and the mixture was stirred for 30 min at room temperature. The mixture was warmed up to 70° C. and a solution of [(1 S,3R)-3-(tert-butoxycarbonylamino)cyclohexyl]methanesulfonate (1.46 g, 3.95 mmol) in DMF (10 mL) was added dropwise. The mixture was stirred at 80° C. for 3 h. The mixture was diluted with ethyl acetate and washed with water. The organic layer was separated, washed with 5% citric acid, brine, dried over sodium sulfate, filtered and concentrated under reduced pressure. The residue was purified by column chromatography (dichloromethane/methanol=98/2 to 9/1 v/v %) to give the title compound (512 mg, 21.4%).

### (c) tert-Butyl N-[(1R,3R)-3-[4-[(2,4-dimethoxyphenyl)methylamino]-3-[4-[[(5-fluoro-2-methoxy-benzoyl)amino]methyl]phenyl]pyrazolo[4,3-c]pyridin-1-yl]cyclohexyl]carbamate

tert-butyl N-[(1R,3R)-3-[4-[(2,4-dimethoxyphenyl)methylamino]-3-iodo-pyrazolo[4,3-c]pyridin-1-yl]cyclohexyl]carbamate (512 mg, 0.84 mmol) was dissolved in dioxane/water=2/1 v/v % (6.3 mL) and potassium carbonate (583 mg, 4.22 mmol) was added. The solution was purged with nitrogen for 5 min and [4-[[(5-fluoro-2-methoxy-benzoyl)amino]methyl]-phenyl]boronic acid (Intermediate BP5) (282 mg, 0.93 mmol) and Pd(dppf)Cl₂.CH₂Cl₂ (35 mg, 0.084 mmol) were added. The reaction mixture was stirred microwave irradiation for 20 min at 120 0C. The reaction mixture was diluted with ethyl acetate and filtered over Decalite™. The filtrate was concentrated under reduced pressure. The residue was purified by column chromatography using SiO₂ and heptane/ethyl acetate=10/0 to 0/10 v/v %. All fractions containing the title compound were collected and concentrated in vacuo to give 484 mg of the title compound (Yield 78%).

### (d) Example 50

This compound was prepared in an analogous manner as described in Method C steps b-g, using tert-butyl N-[(1R,3R)-3-[4-[(2,4-dimethoxyphenyl)methylamino]-3-[4-[[(5-fluoro-2-methoxy-benzoyl)amino]methyl]phenyl]pyrazolo[4,3-c]pyridin-1-yl]cyclohexyl]-carbamate and finally methyl(E)-6-(4,4,5,5-tetramethyl-1,3,2-dioxaborolan-2-yl)hex-5-enoate (Intermediate L6) to afford crude Example 50. Purification was performed using preparative HPLC to afford the title compound (21 mg, 44%). Data: LCMS (B) Rₜ: 9.43 min; m/z 604.4 [M+H]⁺.

The following Examples were synthesized following method D described for Example 50.

| | | | | | | Found | | |
|---|---|---|---|---|---|---|---|---|
| Ex- | Meth- | Inter- | | Rₜ (min) | | Mass | | |
| ample | od | mediate | CHEMISTRY | (LCMS B) | Calc Mass | [M + H]⁺ | Amount | Yield |
| 50 | D | L6 | 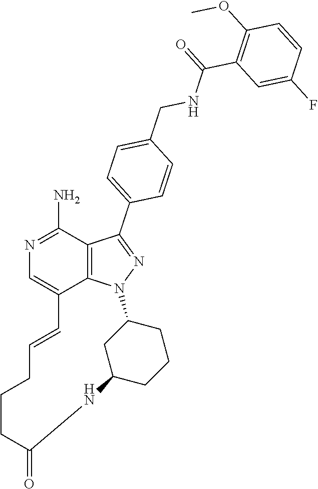 [SMILES: COc1ccc(F)cc1C(=O)NCc1ccc(-c2nn3c4c(cnc(N)c24)/C=C/CCCC(=O)N[C@@H]2CCC[C@@H]3C2)cc1] | 7.98 | 582.27547 | 583.4 | 16.6 mg | 39% |
| 51 | D | L22 | 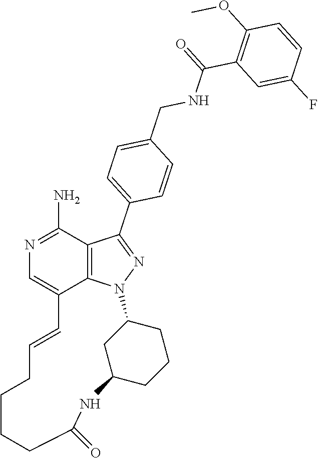 [SMILES: COc1ccc(F)cc1C(=O)NCc1ccc(-c2nn3c4c(cnc(N)c24)/C=C/CCCCC(=O)N[C@@H]2CCC[C@@H]3C2)cc1] | 8.07 | 596.29112 | 597.4 | 27.7 mg | 48.2% |
| 52 | D | L40 | 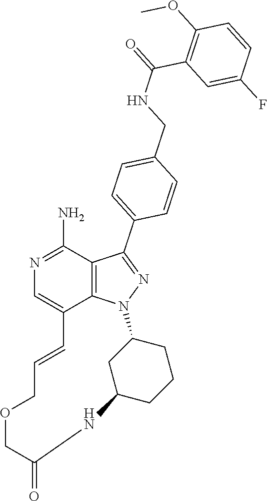 [SMILES: COc1ccc(F)cc1C(=O)NCc1ccc(-c2nn3c4c(cnc(N)c24)/C=C/COCC(=O)N[C@@H]2CCC[C@@H]3C2)cc1] | 7.40 | 584.25473 | 585.4 | 21.1 mg | 33% |
| 53 | D | L26 | 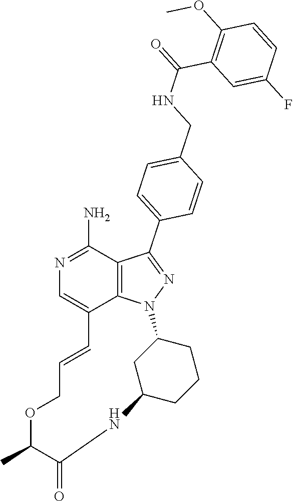 [SMILES: COc1ccc(F)cc1C(=O)NCc1ccc(-c2nn3c4c(cnc(N)c24)/C=C/CO[C@H](C)C(=O)N[C@@H]2CCC[C@@H]3C2)cc1] | 8.12 | 598.27038 | 599.4 | 30.3 mg | 42.4% |

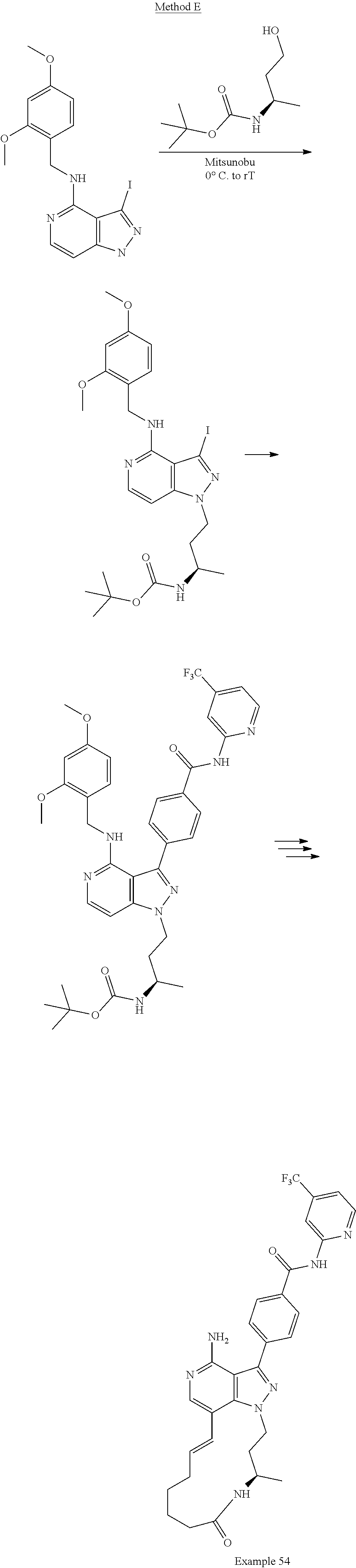 [SMILES: COc1ccc(CNc2nccc3[nH]nc(I)c23)c(OC)c1 ; C[C@H](CCO)NC(=O)OC(C)(C)C ; COc1ccc(CNc2nccc3c2c(I)nn3CC[C@@H](C)NC(=O)OC(C)(C)C)c(OC)c1 ; COc1ccc(CNc2nccc3c2c(-c2ccc(C(=O)Nc4cc(C(F)(F)F)ccn4)cc2)nn3CC[C@@H](C)NC(=O)OC(C)(C)C)c(OC)c1 ; C[C@@H]1CCn2nc(-c3ccc(C(=O)Nc4cc(C(F)(F)F)ccn4)cc3)c3c(N)ncc(c32)/C=C/CCCCC(=O)N1]

### Example 54

### (a) tert-Butyl N-[(1R)-3-[4-[(2,4-dimethoxyphenyl)methylamino]-3-iodo-pyrazolo[4,3-c]pyridin-1-yl]-1-methyl-propyl]carbamate

To an ice-cold (4° C.) suspension of N-[(2,4-dimethoxyphenyl)methyl]-3-iodo-1H-pyrazolo[4,3-c]pyridin-4-amine (Scaffold A) (5 g, 12.19 mmol), tert-butyl N-[(1R)-3-hydroxy-1-methyl-propyl]carbamate (2.77 g, 14.63 mmol) and triphenylphosphine (3.84 g, 14.63) in THE (122 mL) was added dropwise a solution of diisopropyl azodicarboxylate (2.88 mL, 14.63 mmol) in THE (25 mL). The mixture was stirred for 30 min at 4° C. and then allowed to warm to room temperature and stirred o/n. The mixture was concentrated under reduced pressure. The residue was purified by column chromatography using SiO₂ and heptane/ethyl acetate=95/5 to 1/1 v/v %. All fractions containing the title compound were collected and concentrated in vacuo to give 5.73 g of tert-butyl N-[(1R)-3-[4-[(2,4-dimethoxyphenyl)methylamino]-3-iodo-pyrazolo[4,3-c]pyridin-1-yl]-1-methyl-propyl]carbamate (yield: 81%).

### (b) tert-Butyl N-[(1R)-3-[4-[(2,4-dimethoxyphenyl)methylamino]-3-[4-[[4-(trifluoromethyl)-2-pyridyl]carbamoyl]phenyl]pyrazolo[4,3-c]pyridin-1-yl]-1-methyl-propyl]carbamate

tert-Butyl N-[(1R)-3-[4-[(2,4-dimethoxyphenyl)methylamino]-3-iodo-pyrazolo[4,3-c]pyridin-1-yl]-1-methyl-propyl]carbamate (800 mg, 1.38 mmol) was dissolved in dioxane/water=3/1 v/v % (25 mL) and potassium carbonate (951 mg, 4.22 mmol) was added. The solution was purged with nitrogen for 5 min and 4-(4,4,5,5-Tetramethyl-1,3,2-dioxaborolan-2-yl)-N-[4-(trifluoromethyl)-2-pyridyl]benzamide (Intermediate BP1) (593 mg, 6.88 mmol) and Pd(dppf)Cl₂.CH₂Cl₂ (56 mg, 0.07 mmol) were added. The reaction mixture was stirred for 2 h at 100° C. The reaction mixture was diluted with ethyl acetate and filtered over Decalite™. The filtrate was concentrated under reduced pressure. The residue was purified by column chromatography using SiO₂ and heptane/ethyl acetate=95/5 to 0/10 v/v %. All fractions containing the title compound were collected and concentrated in vacuo to give 776 mg of the title compound (Yield 78%).

### (d) Example 54

This compound was prepared in an analogous manner as described in Method C steps b-g, using tert-butyl N-[(1R)-3-[4-[(2,4-dimethoxyphenyl)methylamino]-3-[4-[[4-(trifluoromethyl)-2-pyridyl]carbamoyl]phenyl]pyrazolo[4,3-c]pyridin-1-yl]-1-methyl-propyl]-carbamate and finally methyl(E)-7-(4,4,5,5-tetramethyl-1,3,2-dioxaborolan-2-yl)hept-6-enoate (Intermediate L22) to afford crude Example 54. Purification was performed using preparative HPLC to afford the title compound (23 mg, 36%). Data: LCMS (B) Rₜ: 8.11 min; m/z 578.4 [M+H]⁺.

The following Examples were synthesized following method E described for Example 54.

| | | | | | | Found | | | |
|---|---|---|---|---|---|---|---|---|---|
| Ex- | Meth- | Inter- | | Rₜ (min) | | Mass | | | |
| ample | od | mediate | CHEMISTRY | (LCMS B) | Calc Mass | [M + H]⁺ | Amount | | Yield |
| 54 | E | L22 | 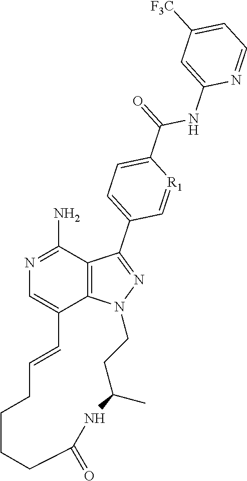 | 8.11 | 577.24131 | 578.4 | 23 | mg | 36% |
| 55 | E | L41 | 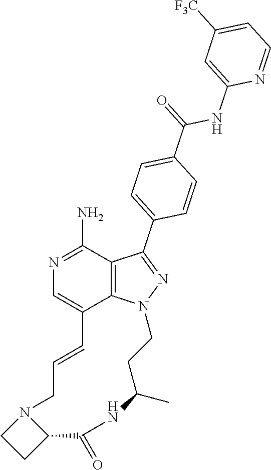 [SMILES: C[C@@H]1CCn2nc(-c3ccc(C(=O)Nc4cc(C(F)(F)F)ccn4)cc3)c3c(N)ncc(c32)/C=C/CN2CC[C@H]2C(=O)N1] | 6.47 | 590.23656 | 591.3 | 2.1 | mg | 24% |
| 56 | E | L40 | 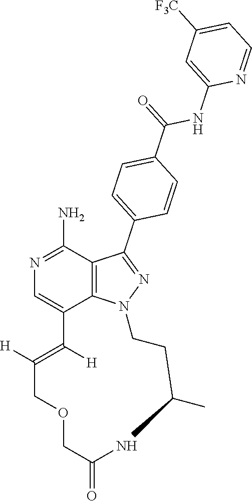 [SMILES: C[C@@H]1CCn2nc(-c3ccc(C(=O)Nc4cc(C(F)(F)F)ccn4)cc3)c3c(N)ncc(c32)/C=C/COCC(=O)N1] | 7.65 | 565.20492 | 566.3 | 24 | mg | 42% |
| 57 | E | L26 | 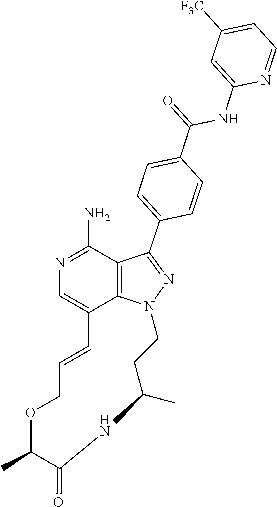 [SMILES: C[C@@H]1CCn2nc(-c3ccc(C(=O)Nc4cc(C(F)(F)F)ccn4)cc3)c3c(N)ncc(c32)/C=C/CO[C@H](C)C(=O)N1] | 8.42 | 579.22057 | 580.5 | 77 | mg | 60% |
| 58 | E | L26 | 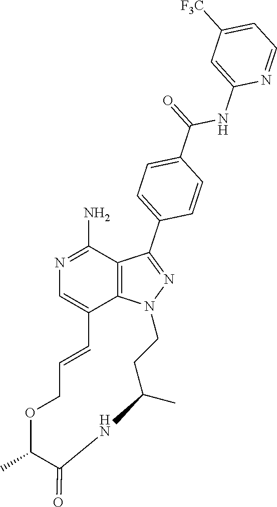 [SMILES: C[C@@H]1CCn2nc(-c3ccc(C(=O)Nc4cc(C(F)(F)F)ccn4)cc3)c3c(N)ncc(c32)/C=C/CO[C@@H](C)C(=O)N1] | 7.97 | 579.22057 | 580.4 | 4 | mg | 3% |
| 59 | E | L5 | 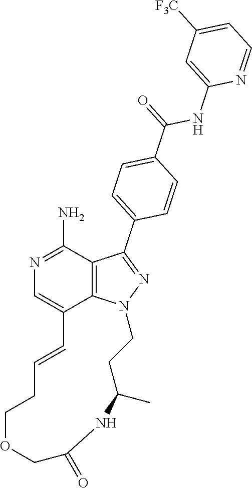 [SMILES: C[C@@H]1CCn2nc(-c3ccc(C(=O)Nc4cc(C(F)(F)F)ccn4)cc3)c3c(N)ncc(c32)/C=C/CCOCC(=O)N1] | 8.29 | 579.22057 | 580.5 | 30 | mg | 41% |
| 60 | E | L15 | 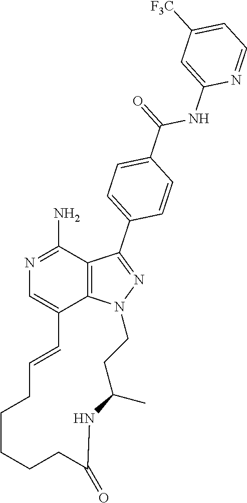 [SMILES: C[C@@H]1CCn2nc(-c3ccc(C(=O)Nc4cc(C(F)(F)F)ccn4)cc3)c3c(N)ncc(c32)/C=C/CCCCCC(=O)N1] | 8.99 | 591.25696 | 592.5 | 17 | mg | 56% |
| 61 | E | L42 | 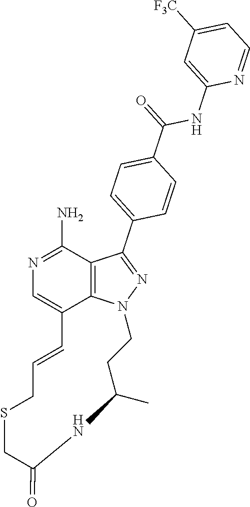 [SMILES: C[C@@H]1CCn2nc(-c3ccc(C(=O)Nc4cc(C(F)(F)F)ccn4)cc3)c3c(N)ncc(c32)/C=C/CSCC(=O)N1] | 8.21 | 581.18208 | 582.4 | 3.7 | mg | 16% |
| 62 | E | L16 | 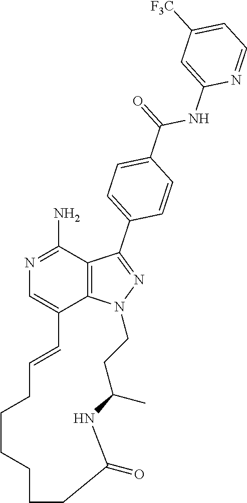 [SMILES: C[C@@H]1CCn2nc(-c3ccc(C(=O)Nc4cc(C(F)(F)F)ccn4)cc3)c3c(N)ncc(c32)/C=C/CCCCCCC(=O)N1] | 9.29 | 605.27261 | 606.5 | 7 | mg | 9% |
| 63 | E | L43 | 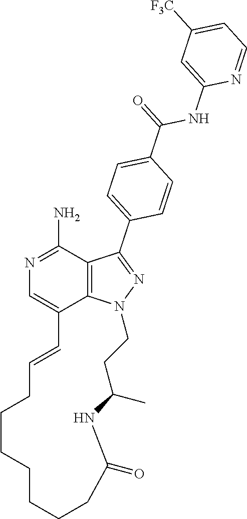 [SMILES: C[C@@H]1CCn2nc(-c3ccc(C(=O)Nc4cc(C(F)(F)F)ccn4)cc3)c3c(N)ncc(c32)/C=C/CCCCCCCC(=O)N1] | 9.48 | 619.28826 | 620.5 | 29 | mg | 36% |
| 64 | E | L44 | 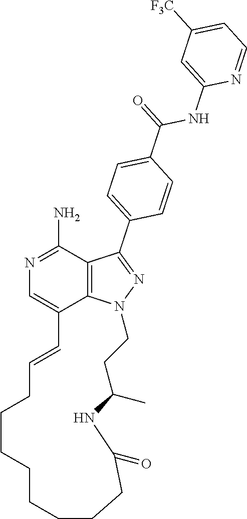 [SMILES: C[C@@H]1CCn2nc(-c3ccc(C(=O)Nc4cc(C(F)(F)F)ccn4)cc3)c3c(N)ncc(c32)/C=C/CCCCCCCCC(=O)N1] | 10.05 | 633.30391 | 634.5 | 13 | mg | 17% |
| 65 | E | L42 | 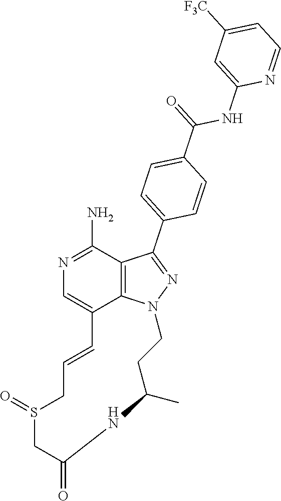 [SMILES: C[C@@H]1CCn2nc(-c3ccc(C(=O)Nc4cc(C(F)(F)F)ccn4)cc3)c3c(N)ncc(c32)/C=C/CS(=O)CC(=O)N1] | 7.49 | 597.17699 | 598.3 | 3.8 | mg | 11% |
| 66 | E | L9 | 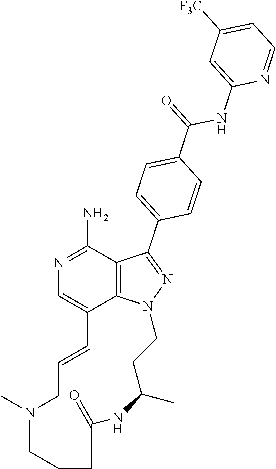 [SMILES: C[C@@H]1CCn2nc(-c3ccc(C(=O)Nc4cc(C(F)(F)F)ccn4)cc3)c3c(N)ncc(c32)/C=C/CN(C)CCCC(=O)N1] | 6.71 | 606.26786 | 607.4 | 23 | mg | 47% |
| 67 | E | L14 | 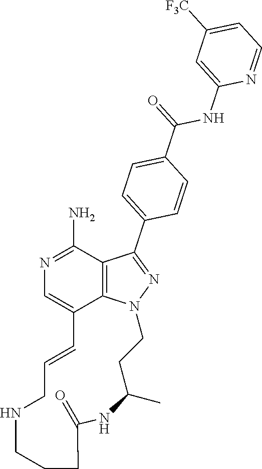 [SMILES: C[C@@H]1CCn2nc(-c3ccc(C(=O)Nc4cc(C(F)(F)F)ccn4)cc3)c3c(N)ncc(c32)/C=C/CNCCCC(=O)N1] | 3.25 (LCMS A) | 592.25221 | 593.4 | 67 | mg | 87% |
| 68 | E | L31 | 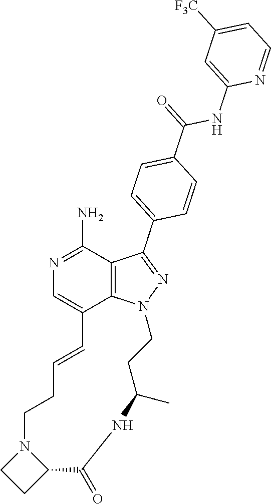 [SMILES: C[C@@H]1CCn2nc(-c3ccc(C(=O)Nc4cc(C(F)(F)F)ccn4)cc3)c3c(N)ncc(c32)/C=C/CCN2CC[C@H]2C(=O)N1] | 6.58 | 604.25221 | 605.3 | 20 | mg | 67% |
| 69 | E | L24 | 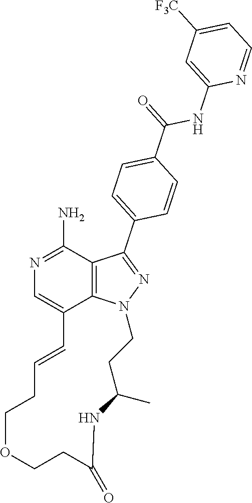 [SMILES: C[C@@H]1CCn2nc(-c3ccc(C(=O)Nc4cc(C(F)(F)F)ccn4)cc3)c3c(N)ncc(c32)/C=C/CCOCCC(=O)N1] | 8.26 | 593.23622 | 594.3 | 38 | mg | 75% |
| 70 | E | L18 | 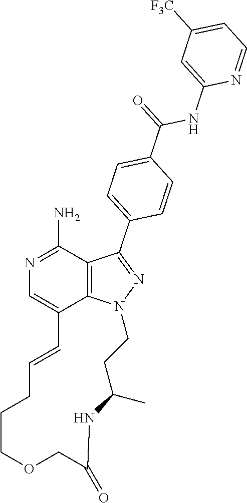 [SMILES: C[C@@H]1CCn2nc(-c3ccc(C(=O)Nc4cc(C(F)(F)F)ccn4)cc3)c3c(N)ncc(c32)/C=C/CCCOCC(=O)N1] | 8.19 | 593.23622 | 594.3 | 12 | mg | 59% |
| 71 | E | L23 | 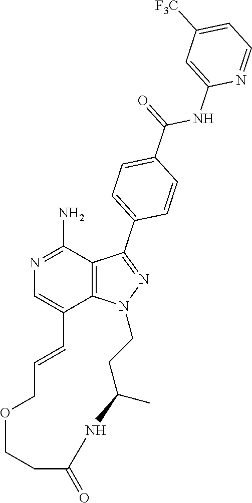 [SMILES: C[C@@H]1CCn2nc(-c3ccc(C(=O)Nc4cc(C(F)(F)F)ccn4)cc3)c3c(N)ncc(c32)/C=C/COCCC(=O)N1] | 7.93 | 579.22057 | 580.3 | 6.4 | mg | 16% |
| 72 | E | L45 | 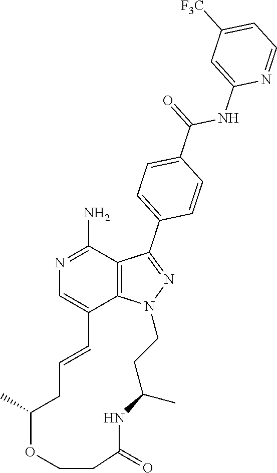 [SMILES: C[C@@H]1CCn2nc(-c3ccc(C(=O)Nc4cc(C(F)(F)F)ccn4)cc3)c3c(N)ncc(c32)/C=C/C[C@@H](C)OCCC(=O)N1] | 8.32 | 607.25187 | 608.4 | 6 | mg | 18.7% |
| 73 | E | L45 | 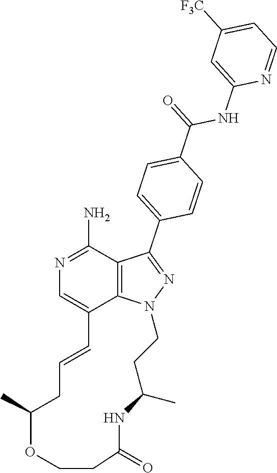 [SMILES: C[C@@H]1CCn2nc(-c3ccc(C(=O)Nc4cc(C(F)(F)F)ccn4)cc3)c3c(N)ncc(c32)/C=C/C[C@H](C)OCCC(=O)N1] | 8.54 | 607.25187 | 608.4 | 6 | mg | 18.7% |
| 74 | E | L36 | 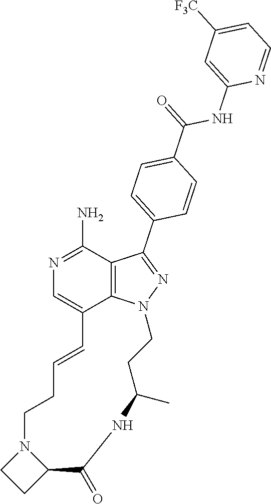 [SMILES: C[C@@H]1CCn2nc(-c3ccc(C(=O)Nc4cc(C(F)(F)F)ccn4)cc3)c3c(N)ncc(c32)/C=C/CCN2CC[C@@H]2C(=O)N1] | 6.70 | 604.25221 | 605.4 | 16 | mg | 74% |
| 75 | E | L10 | 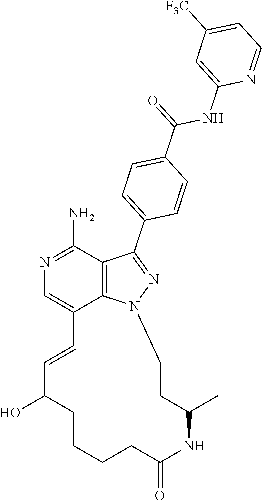 | 8.04 | 607.25187 | 608.4 | 2.7 | mg | 12% |
| 76 | E | L6 | 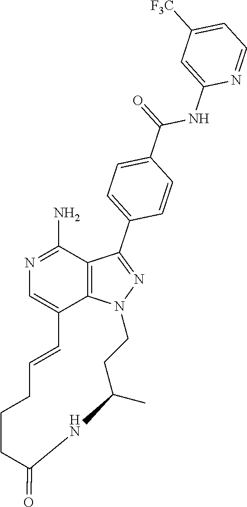 [SMILES: C[C@@H]1CCn2nc(-c3ccc(C(=O)Nc4cc(C(F)(F)F)ccn4)cc3)c3c(N)ncc(c32)/C=C/CCCC(=O)N1] | 8.18 | 563.22566 | 564.4 | 2.4 | mg | 22% |

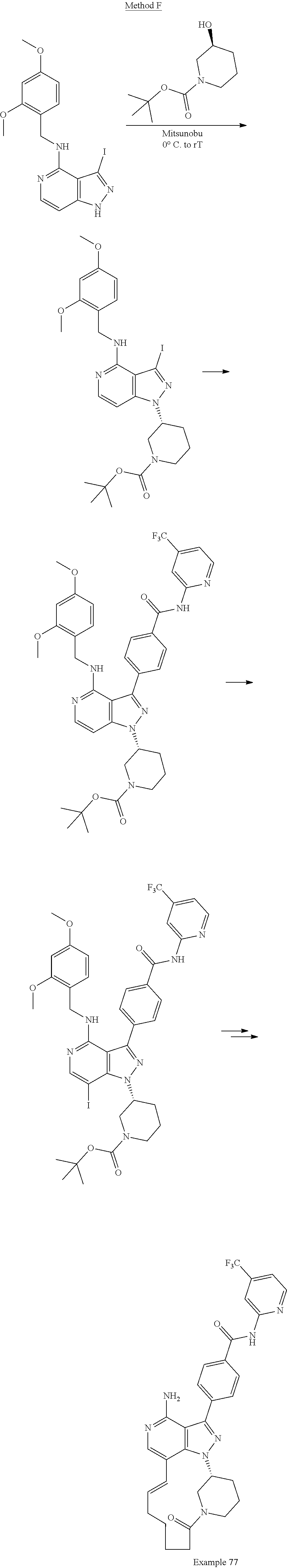 [SMILES: COc1ccc(CNc2nccc3[nH]nc(I)c23)c(OC)c1 ; CC(C)(C)OC(=O)N1CCC[C@H](O)C1 ; COc1ccc(CNc2nccc3c2c(I)nn3[C@@H]2CCCN(C(=O)OC(C)(C)C)C2)c(OC)c1 ; COc1ccc(CNc2nccc3c2c(-c2ccc(C(=O)Nc4cc(C(F)(F)F)ccn4)cc2)nn3[C@@H]2CCCN(C(=O)OC(C)(C)C)C2)c(OC)c1 ; COc1ccc(CNc2ncc(I)c3c2c(-c2ccc(C(=O)Nc4cc(C(F)(F)F)ccn4)cc2)nn3[C@@H]2CCCN(C(=O)OC(C)(C)C)C2)c(OC)c1 ; Nc1ncc2c3c1c(-c1ccc(C(=O)Nc4cc(C(F)(F)F)ccn4)cc1)nn3[C@@H]1CCCN(C1)C(=O)CCCC/C=C/2]

### Example 77

### (a) tert-Butyl(3R)-3-[4-[(2,4-dimethoxyphenyl)methylamino]-3-iodo-pyrazolo[4,3-c]pyridin-1-yl]piperidine-1-carboxylate

To an ice-cold (4° C.) suspension of N-[(2,4-dimethoxyphenyl)methyl]-3-iodo-1H-pyrazolo[4,3-c]pyridin-4-amine (Scaffold A) (1 g, 2.44 mmol), tert-butyl(3S)-3-hydroxy-piperidine-1-carboxylate (638 mg, 3.17 mmol) and triphenylphosphine (1 g, 3.83) in toluene (19 mL) was added dropwise a solution of di-2-methoxyethyl azodicarboxylate (792 mg, 3.83 mmol) 10 in toluene (5 mL). The mixture was stirred for 30 min at 4° C. and then allowed to warm to room temperature and stirred o/n. The mixture was concentrated under reduced pressure. The residue was purified by column chromatography using SiO₂ and heptane/ethyl acetate=95/5 to 45/55 v/v %. All fractions containing the title compound were collected and concentrated in vacuo to give 480 mg of tert-butyl(3R)-3-[4-[(2,4-dimethoxyphenyl)methylamino]-3-iodo-pyrazolo[4,3-c]pyridin-1-yl]piperidine-1-carboxylate (yield: 33%).

### (b) tert-Butyl(3R)-3-[4-[(2,4-dimethoxyphenyl)methylamino]-3-[4-[[4-(trifluoromethyl)-2-pyridyl]carbamoyl]phenyl]pyrazolo[4,3-c]pyridin-1-yl]piperidine-1-carboxylate

tert-Butyl(3R)-3-[4-[(2,4-dimethoxyphenyl)methylamino]-3-iodo-pyrazolo[4,3-c]pyridin-1-yl]piperidine-1-carboxylate (830 mg, 1.40 mmol) was dissolved in dioxane/water=4/1 v/v % (15 mL) and potassium carbonate (580 mg, 4.2 mmol) was added. The solution was purged with nitrogen for 5 min and 4-(4,4,5,5-Tetramethyl-1,3,2-dioxaborolan-2-yl)-N-[4-(trifluorome-thyl)-2-pyridyl]benzamide (Intermediate BP1) (603 mg, 1.54 mmol) and Pd(dppf)Cl₂.CH₂Cl₂ (57 mg, 0.07 mmol) were added. The reaction mixture was stirred for 1 h at 80° C. The reaction mixture was diluted with ethyl acetate and filtered over Decalite™. The filtrate was concentrated under reduced pressure to give 1.1 g of the title compound (quant.).

### (c) tert-Butyl(3R)-3-[4-[(2,4-dimethoxyphenyl)methylamino]-7-iodo-3-[4-[[4-(trifluoromethyl)-2-pyridyl]carbamoyl]phenyl]pyrazolo[4,3-c]pyridin-1-yl]piperidine-1-carboxylate

To a cold (0° C.) solution of tert-butyl(3R)-3-[4-[(2,4-dimethoxyphenyl)methylamino]-3-[4-[[4-(trifluoromethyl)-2-pyridyl]carbamoyl]phenyl]pyrazolo[4,3-c]pyridin-1-yl]piperidine-1-carboxylate (1.1 g, 1.40 mmol) in DMF (15 mL) was added N-iodosuccinimide (346 mg, 1.54 mmol). The reaction mixture was stirred for 2 h. allowing the mixture to warm up to room temperature. The mixture was diluted with ethyl acetate and washed with sodium thiosulfate solution, water, brine, dried over sodium sulfate, filtered and concentrated in vacuo. The crude product was purified by column chromatography using SiO₂ and heptane/ethyl acetate=95/5 to 1/1 v/v %. All fractions containing the title compound were collected and concentrated in vacuo to give 1.06 g of the title compound (Yield: 88%).

### (d) Example 77

This compound was prepared in an analogous manner as described in Method C steps c-g, using tert-butyl(3R)-3-[4-[(2,4-dimethoxyphenyl)methylamino]-7-iodo-3-[4-[[4-(trifluoromethyl)-2-pyridyl]carbamoyl]phenyl]pyrazolo[4,3-c]pyridin-1-yl]piperidine-1-carboxylate and finally methyl(E)-7-(4,4,5,5-tetramethyl-1,3,2-dioxaborolan-2-yl)hept-6-enoate (Intermediate L22) to afford crude Example 77. Purification was performed using preparative HPLC to afford the title compound (14 mg, 18.3%). Data: LCMS (B) Rₜ: 8.79 min; m/z 590.5 [M+H]⁺.

The following Examples were synthesized following method F described for Example 77.

| | | | | | | Found | | |
|---|---|---|---|---|---|---|---|---|
| | | | | Rₜ (min) | | Mass | | |
| Example | Method | Intermediate | CHEMISTRY | (LCMS B) | Calc Mass | [M + H]⁺ | Amount | Yield |
| 77 | F | L22 | 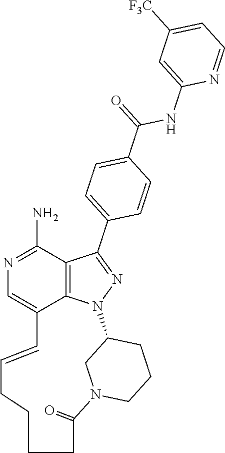 [SMILES: Nc1ncc2c3c1c(-c1ccc(C(=O)Nc4cc(C(F)(F)F)ccn4)cc1)nn3[C@@H]1CCCN(C1)C(=O)CCCC/C=C/2] | 8.79 | 589.24131 | 590.5 | 14 mg | 18.3% |
| 78 | F | L15 | 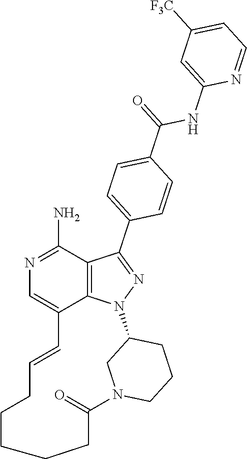 [SMILES: Nc1ncc2c3c1c(-c1ccc(C(=O)Nc4cc(C(F)(F)F)ccn4)cc1)nn3[C@@H]1CCCN(C1)C(=O)CCCCC/C=C/2] | 9.36 | 603.25696 | 604.5 | 5 mg | 8.3% |
| 79 | F | L16 | 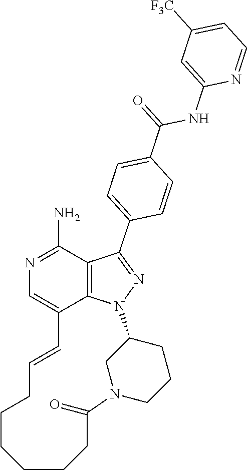 [SMILES: Nc1ncc2c3c1c(-c1ccc(C(=O)Nc4cc(C(F)(F)F)ccn4)cc1)nn3[C@@H]1CCCN(C1)C(=O)CCCCCC/C=C/2] | 10.15 | 617.27261 | 618.5 | 10 mg | 16.2% |
| 80 | F | L43 | 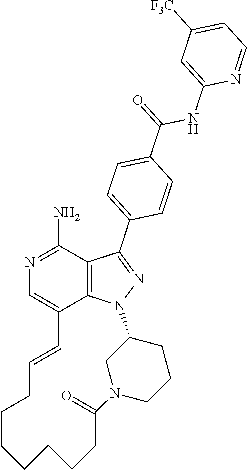 [SMILES: Nc1ncc2c3c1c(-c1ccc(C(=O)Nc4cc(C(F)(F)F)ccn4)cc1)nn3[C@@H]1CCCN(C1)C(=O)CCCCCCC/C=C/2] | 10.44 | 631.28826 | 632.5 | 16 mg | 25.4% |
| 81 | F | L44 | 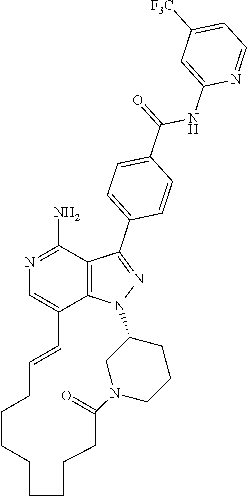 [SMILES: Nc1ncc2c3c1c(-c1ccc(C(=O)Nc4cc(C(F)(F)F)ccn4)cc1)nn3[C@@H]1CCCN(C1)C(=O)CCCCCCCC/C=C/2] | 11.26 | 645.30391 | 646.5 | 16 mg | 24.3% |
| 82 | F | L21 | 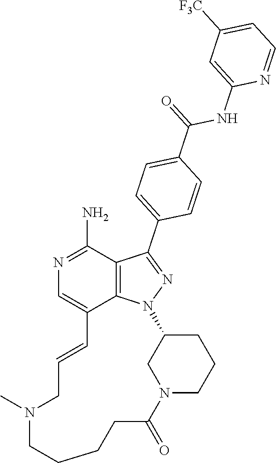 [SMILES: CN1C/C=C/c2cnc(N)c3c(-c4ccc(C(=O)Nc5cc(C(F)(F)F)ccn5)cc4)nn(c23)[C@@H]2CCCN(C2)C(=O)CCCC1] | 7.06 | 632.28351 | 633.4 | 6.3 mg | 7.7% |
| 83 | F | L46 | 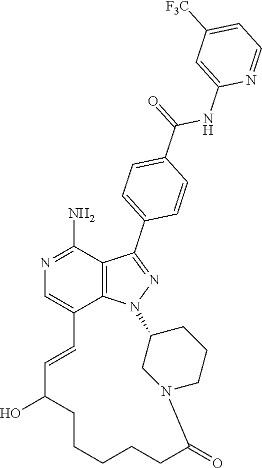 [SMILES: Nc1ncc2c3c1c(-c1ccc(C(=O)Nc4cc(C(F)(F)F)ccn4)cc1)nn3[C@@H]1CCCN(C1)C(=O)CCCCCC(O)/C=C/2] | 8.88 | 633.26752 | 634.4 | 6.8 mg | 6.3% |

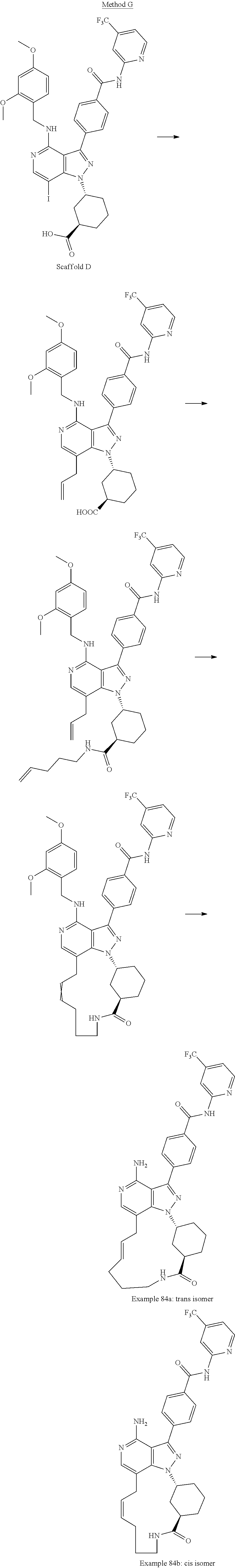

### Example 84

### (a) (1R,3R)-3-[7-allyl-4-[(2,4-dimethoxyphenyl)methylamino]-3-[4-[[4-(trifluoromethyl)-2-pyridyl]carbamoyl]phenyl]pyrazolo[4,3-c]pyridin-1-yl]cyclohexanecarboxylic acid

A mixture of (1R,3R)-3-[4-[(2,4-dimethoxyphenyl)methylamino]-7-iodo-3-[4-[[4-(trifluoromethyl)-2-pyridyl]carbamoyl]phenyl]pyrazolo[4,3-c]pyridin-1-yl]cyclohexanecarboxylic acid (Scaffold D) (736 mg, 0.92 mmol), 2-allyl-4,4,5,5-tetramethyl-1,3,2-dioxaborolane (541 mg, 3.22 mmol) and cesium fluoride (559 mg, 3.68 mmol) in dioxane (9 mL) was degassed with nitrogen. CataCXium® A Pd G3 (29 mg, 0.04 mmol) was added the reaction mixture was stirred for 2 h at 65° C. After cooling, ethyl acetate was added and the mixture was stirred for 5 min. The mixture was filtered over Decalite™ and the filtrate was washed with 5% citric acid solution, water and brine. The organic layer was separated, dried over sodium sulfate, filtered and then concentrated under reduced pressure. The residue was triturated with diethyl ether to give 948 mg of the title compound (yield: 101%).

### (b) 4-[7-allyl-4-[(2,4-dimethoxyphenyl)methylamino]-1-[(1R,3R)-3-(pent-4-enylcarbamoyl)cyclo-hexyl]pyrazolo[4,3-c]pyridin-3-yl]-N-[4-(trifluoromethyl)-2-pyridyl]benzamide

(1R,3R)-3-[7-allyl-4-[(2,4-dimethoxyphenyl)methylamino]-3-[4-[[4-(trifluoromethyl)-2-pyridyl]carbamoyl]phenyl]pyrazolo[4,3-c]pyridin-1-yl]cyclohexanecarboxylic acid (100 mg, 0.14 mmol) and pent-4-en-1-amine hydrochloride (20 mg, 0.18 mmol) were suspended in DMF (1.4 ml). N-Ethylmorpholine (54 μl, 0.16 mmol) and HATU (59 mg, 0.15 mmol) were added subsequently and the mixture stirred at room temperature o/n. The mixture was washed with 5% NaHCO₃-solution/brine. The organic layer was separated and washed with 5% aq. Citric acid solution, water, brine, dried over sodium sulfate, filtered and concentrated in vacuo. The crude product was purified by column chromatography using SiO₂ and heptane/ethyl acetate=10/0 to 1/4 v/v %. All fractions containing the title compound were collected and concentrated in vacuo to give 30 mg of the title compound (Yield: 27%).

### (c) mixture of DMB-protected trans/cis macrocycle

A solution of 4-[7-allyl-4-[(2,4-dimethoxyphenyl)methylamino]-1-[(1R,3R)-3-(pent-4-enylcarbamoyl)-cyclohexyl]pyrazolo[4,3-c]pyridin-3-yl]-N-[4-(trifluoromethyl)-2-pyridyl]benzamide (30 mg, 0.033 mmol) was dissolved in dichloromethane (1 mL) and added dropwise to a solution of Hoveyda-Grubbs Catalyst 2nd Generation (2.4 mg, 3.8 μmol) in dichloromethane (2.8 mL). The resulting solution was stirred 24 h at reflux in a sealed tube (5 mL). This procedure was repeated. The mixture was concentrated under reduced pressure and the residue was purified by column chromatography using SiO₂ and dichloromethane/ethyl acetate=10/0 to 0/10 v/v %. All fractions containing the title compound were collected and concentrated in vacuo to give 28 mg of a mixture of DMB-protected trans- and cis-macrocycle. (quantitative yield).

### (d) Example 84a and Example 84b

Deprotection of the DMB-protected trans/cis-macrocycle was performed as described in Example 6 step e using TFA/TIS/water=90/5/5 v/v %. Purification was performed using preparative HPLC to afford the two separated trans and cis-isomers of the title compound. Example 84a is the first eluting isomer, which corresponds with the trans-isomer (11 mg, 47.4%) Data: LCMS (B) Rₜ: 9.05 min; m/z 604.5 [M+H]⁺. Example 84b is the last eluting isomer, which corresponds with the cis-isomer (3 mg, 12.3%) Data: LCMS (B) Rₜ: 9.53 min; m/z 604.5 [M+H]⁺.

The following Examples were synthesized following method G described for Example 84.

| | | | | | | Found | | |
|---|---|---|---|---|---|---|---|---|
| | | | | Rₜ (min) | | Mass | | |
| Example | Method | Intermediate | CHEMISTRY | (LCMS B) | Calc Mass | [M + H]⁺ | Amount | Yield |
| 84a | G | Commerc | 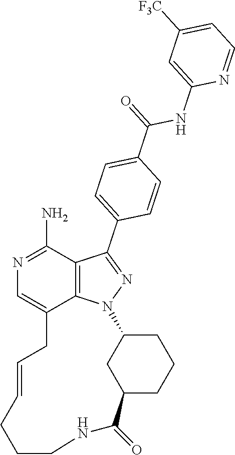 [SMILES: Nc1ncc2c3c1c(-c1ccc(C(=O)Nc4cc(C(F)(F)F)ccn4)cc1)nn3[C@@H]1CCC[C@H](C1)C(=O)NCCC/C=C/C2] | 9.04 | 603.25696 | 604.5 | 11 mg | 47.4% |
| 84b | G | Commerc | 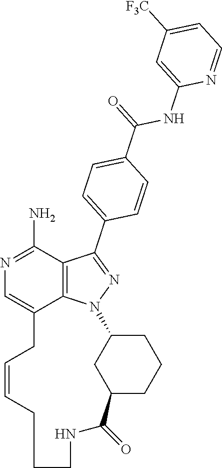 [SMILES: Nc1ncc2c3c1c(-c1ccc(C(=O)Nc4cc(C(F)(F)F)ccn4)cc1)nn3[C@@H]1CCC[C@H](C1)C(=O)NCCC/C=C\C2] | 9.53 | 603.25696 | 604.5 | 3 mg | 12.3% |
| 85a | G | Commerc | 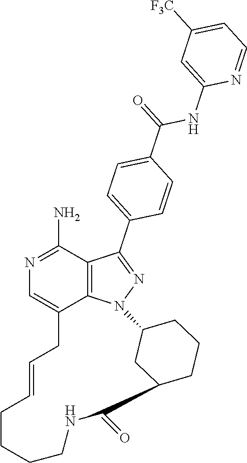 [SMILES: Nc1ncc2c3c1c(-c1ccc(C(=O)Nc4cc(C(F)(F)F)ccn4)cc1)nn3[C@@H]1CCC[C@H](C1)C(=O)NCCCC/C=C/C2] | 9.76 | 617.27261 | 618.5 | 16 mg | 32.2% |
| 85b | G | Commerc | 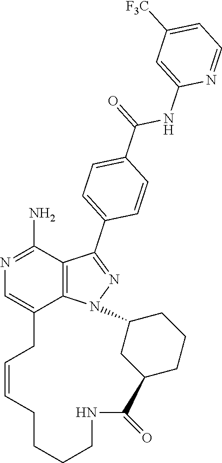 [SMILES: Nc1ncc2c3c1c(-c1ccc(C(=O)Nc4cc(C(F)(F)F)ccn4)cc1)nn3[C@@H]1CCC[C@H](C1)C(=O)NCCCC/C=C\C2] | 10.34 | 617.27261 | 618.5 | 16 mg | 32.2% |
| 86 | G | Commerc | 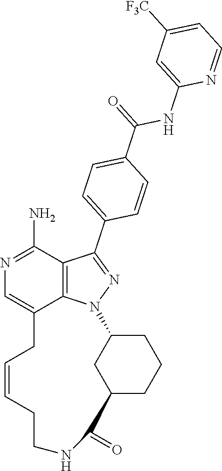 [SMILES: Nc1ncc2c3c1c(-c1ccc(C(=O)Nc4cc(C(F)(F)F)ccn4)cc1)nn3[C@@H]1CCC[C@H](C1)C(=O)NCC/C=C\C2] | 9.09 | 589.24131 | 590.4 | 28 mg | 52% |
| 87a | G | Commerc | 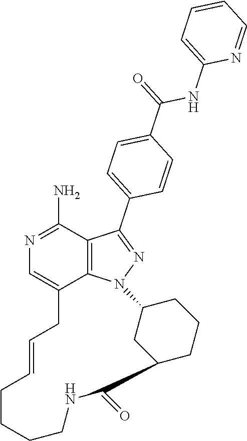 [SMILES: Nc1ncc2c3c1c(-c1ccc(C(=O)Nc4ccccn4)cc1)nn3[C@@H]1CCC[C@H](C1)C(=O)NCCCC/C=C/C2] | 7.11 | 549.28522 | 550.5 | 13 mg | 33% |
| 87b | G | Commerc | 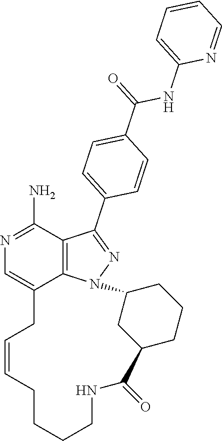 [SMILES: Nc1ncc2c3c1c(-c1ccc(C(=O)Nc4ccccn4)cc1)nn3[C@@H]1CCC[C@H](C1)C(=O)NCCCC/C=C\C2] | 7.79 | 549.28522 | 550.5 | 4 mg | 10% |
| 88a | G | Commerc | 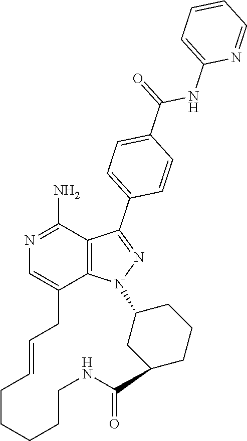 [SMILES: Nc1ncc2c3c1c(-c1ccc(C(=O)Nc4ccccn4)cc1)nn3[C@@H]1CCC[C@H](C1)C(=O)NCCCCC/C=C/C2] | 7.70 | 563.30087 | 564.5 | 9 mg | 20% |
| 88b | G | Commerc | 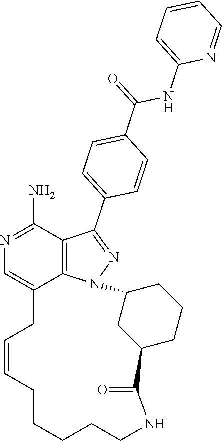 [SMILES: Nc1ncc2c3c1c(-c1ccc(C(=O)Nc4ccccn4)cc1)nn3[C@@H]1CCC[C@H](C1)C(=O)NCCCCC/C=C\C2] | 8.11 | 563.30087 | 564.5 | 5 mg | 11% |
| 89a | G | Commerc | 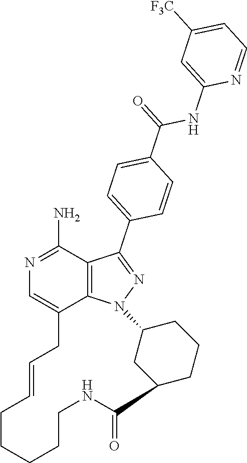 [SMILES: Nc1ncc2c3c1c(-c1ccc(C(=O)Nc4cc(C(F)(F)F)ccn4)cc1)nn3[C@@H]1CCC[C@H](C1)C(=O)NCCCCC/C=C/C2] | 10.18 | 631.28826 | 632.5 | 0.9 mg | 2% |
| 89b | G | Commerc | 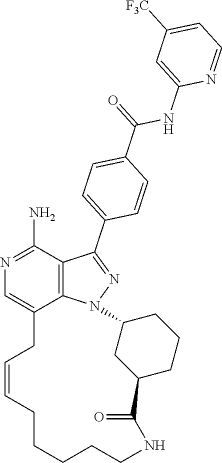 [SMILES: Nc1ncc2c3c1c(-c1ccc(C(=O)Nc4cc(C(F)(F)F)ccn4)cc1)nn3[C@@H]1CCC[C@H](C1)C(=O)NCCCCC/C=C\C2] | 10.54 | 631.28826 | 632.5 | 0.8 mg | 1% |
| 90 | G | Commerc | 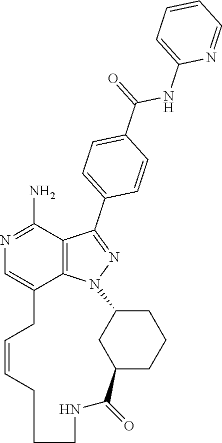 [SMILES: Nc1ncc2c3c1c(-c1ccc(C(=O)Nc4ccccn4)cc1)nn3[C@@H]1CCC[C@H](C1)C(=O)NCCC/C=C\C2] | 6.83 | 535.26957 | 536.5 | 0.9 mg | 2% |
| 91 | G | Commerc | 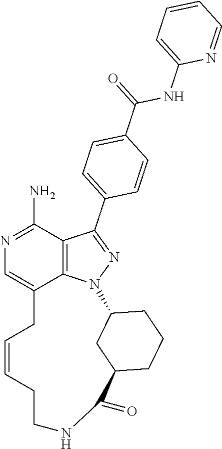 [SMILES: Nc1ncc2c3c1c(-c1ccc(C(=O)Nc4ccccn4)cc1)nn3[C@@H]1CCC[C@H](C1)C(=O)NCC/C=C\C2] | 6.51 | 521.25392 | 522.5 | 1.1 mg | 1.4% |
| 92 | G | Commerc | 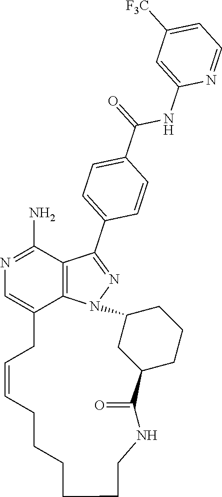 [SMILES: Nc1ncc2c3c1c(-c1ccc(C(=O)Nc4cc(C(F)(F)F)ccn4)cc1)nn3[C@@H]1CCC[C@H](C1)C(=O)NCCCCCC/C=C\C2] | 11.33 | 645.30391 | 646.5 | 1 mg | 2% |
| 93 | G | Commerc | 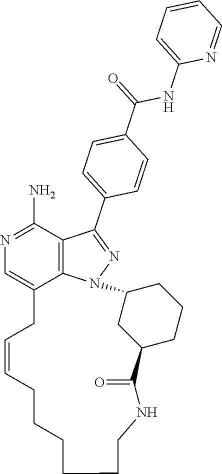 [SMILES: Nc1ncc2c3c1c(-c1ccc(C(=O)Nc4ccccn4)cc1)nn3[C@@H]1CCC[C@H](C1)C(=O)NCCCCCC/C=C\C2] | 8.82 | 577.31652 | 578.5 | 11 mg | 30% |

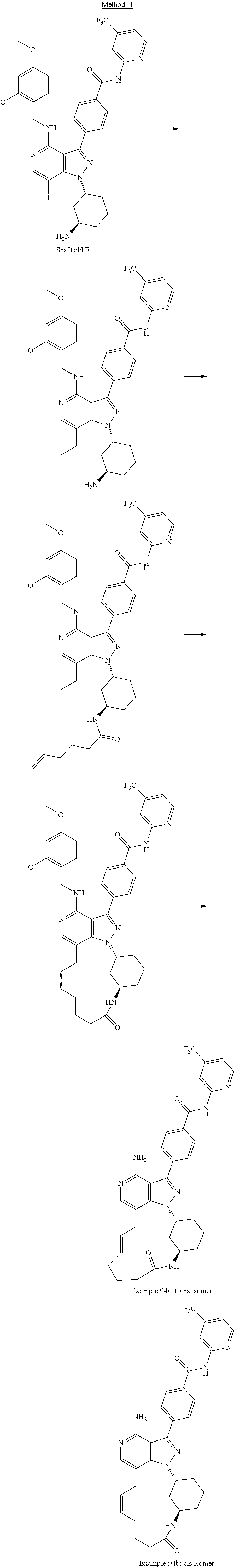

### Example 94

This compound was prepared in an analogous manner as described in Method G steps a-d, starting from 4-[1-[(1R,3R)-3-aminocyclohexyl]-4-[(2,4-dimethoxyphenyl)methyl-amino]-7-iodo-pyrazolo[4,3-c]pyridin-3-yl]-N-[4-(trifluoromethyl)-2-pyridyl]benzamide (Scaffold E) to afford crude Example 94. Purification was performed using preparative HPLC to afford the two separated trans and cis-isomers of the title compound. Example 94a is the first eluting isomer, which corresponds with the trans-isomer (8 mg, 35.6%) Data: LCMS (B) Rₜ: 8.92 min; m/z 604.5 [M+H]⁺. Example 94b is the last eluting isomer, which corresponds with the cis-isomer (3 mg, 13.4%) Data: LCMS (B) Rₜ: 9.23 min; m/z 604.5 [M+H]⁺.

The following Examples were synthesized following method H described for Example 94.

| | | | | | | Found | | |
|---|---|---|---|---|---|---|---|---|
| | | | | Rₜ (min) | | Mass | | |
| Example | Method | Intermediate | CHEMISTRY | (LCMS B) | Calc Mass | [M + H]⁺ | Amount | Yield |
| 94a | H | Commerc | 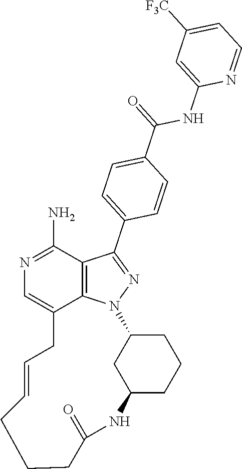 [SMILES: Nc1ncc2c3c1c(-c1ccc(C(=O)Nc4cc(C(F)(F)F)ccn4)cc1)nn3[C@@H]1CCC[C@H](C1)NC(=O)CCC/C=C/C2] | 8.92 | 603.25696 | 604.5 | 8 mg | 35.6% |
| 94b | H | Commerc | 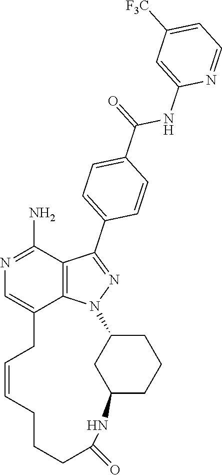 [SMILES: Nc1ncc2c3c1c(-c1ccc(C(=O)Nc4cc(C(F)(F)F)ccn4)cc1)nn3[C@@H]1CCC[C@H](C1)NC(=O)CCC/C=C\C2] | 9.23 | 603.25696 | 604.5 | 3 mg | 13.4% |
| 95 | H | Commerc | 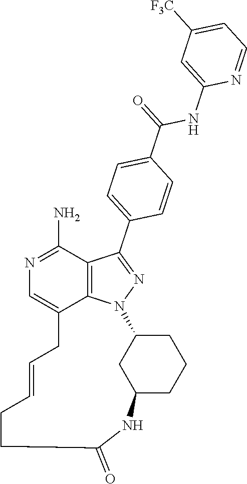 [SMILES: Nc1ncc2c3c1c(-c1ccc(C(=O)Nc4cc(C(F)(F)F)ccn4)cc1)nn3[C@@H]1CCC[C@H](C1)NC(=O)CC/C=C/C2] | 8.67 | 589.24131 | 590.5 | 34 mg | 64.2% |
| 96a | H | Commerc | 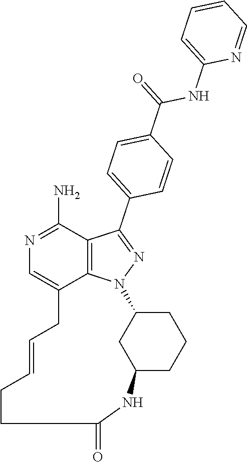 [SMILES: Nc1ncc2c3c1c(-c1ccc(C(=O)Nc4ccccn4)cc1)nn3[C@@H]1CCC[C@H](C1)NC(=O)CC/C=C/C2] | 6.04 | 521.25392 | 522.5 | 13 mg | 18% |
| 96b | H | Commerc | 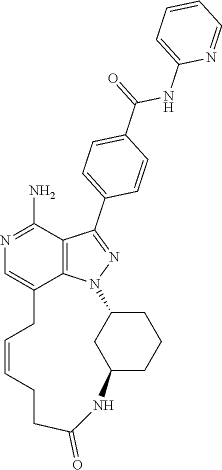 [SMILES: Nc1ncc2c3c1c(-c1ccc(C(=O)Nc4ccccn4)cc1)nn3[C@@H]1CCC[C@H](C1)NC(=O)CC/C=C\C2] | 6.31 | 521.25392 | 522.4 | 0.4 mg | 0.5% |
| 97a | H | Commerc | 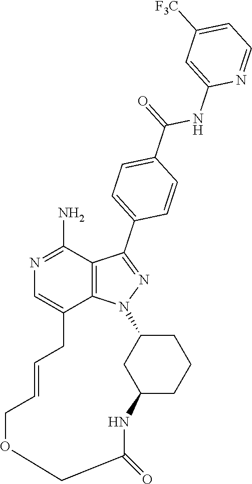 [SMILES: Nc1ncc2c3c1c(-c1ccc(C(=O)Nc4cc(C(F)(F)F)ccn4)cc1)nn3[C@@H]1CCC[C@H](C1)NC(=O)COC/C=C/C2] | 8.47 | 605.23622 | 606.5 | 16 mg | 32.2% |
| 97b | H | Commerc | 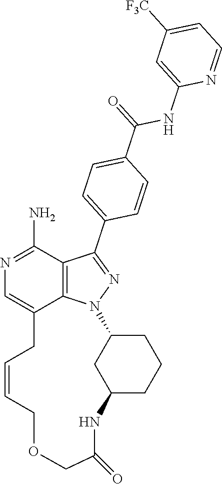 [SMILES: Nc1ncc2c3c1c(-c1ccc(C(=O)Nc4cc(C(F)(F)F)ccn4)cc1)nn3[C@@H]1CCC[C@H](C1)NC(=O)COC/C=C\C2] | 8.57 | 605.23622 | 606.5 | 6 mg | 12.1% |
| 98a | H | Commerc | 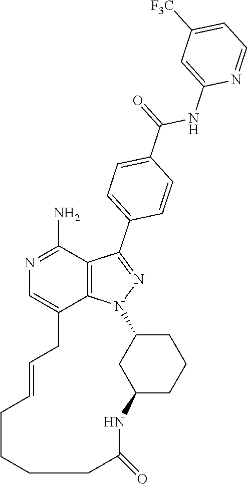 [SMILES: Nc1ncc2c3c1c(-c1ccc(C(=O)Nc4cc(C(F)(F)F)ccn4)cc1)nn3[C@@H]1CCC[C@H](C1)NC(=O)CCCC/C=C/C2] | 8.91 | 617.27261 | 618.5 | 8 mg | 24.4% |
| 98b | H | Commerc | 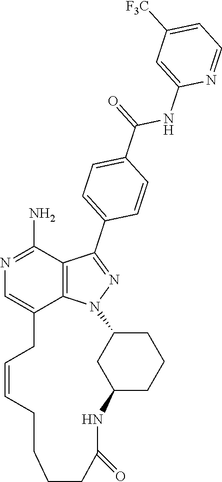 [SMILES: Nc1ncc2c3c1c(-c1ccc(C(=O)Nc4cc(C(F)(F)F)ccn4)cc1)nn3[C@@H]1CCC[C@H](C1)NC(=O)CCCC/C=C\C2] | 9.60 | 617.27261 | 618.5 | 6 mg | 15.5% |
| 99a | H | Commerc | 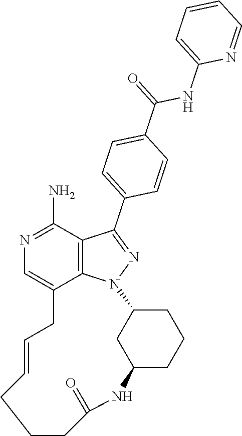 [SMILES: Nc1ncc2c3c1c(-c1ccc(C(=O)Nc4ccccn4)cc1)nn3[C@@H]1CCC[C@H](C1)NC(=O)CCC/C=C/C2] | 6.26 | 535.26957 | 536.5 | 25 mg | 27% |
| 99b | H | Commerc | 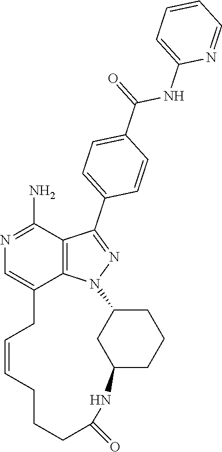 [SMILES: Nc1ncc2c3c1c(-c1ccc(C(=O)Nc4ccccn4)cc1)nn3[C@@H]1CCC[C@H](C1)NC(=O)CCC/C=C\C2] | 6.60 | 535.26957 | 536.5 | 4 mg | 4% |
| 100a | H | Commerc | 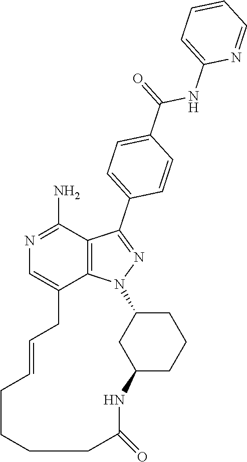 [SMILES: Nc1ncc2c3c1c(-c1ccc(C(=O)Nc4ccccn4)cc1)nn3[C@@H]1CCC[C@H](C1)NC(=O)CCCC/C=C/C2] | 6.42 | 549.28522 | 550.5 | 18 mg | 20% |
| 100b | H | Commerc | 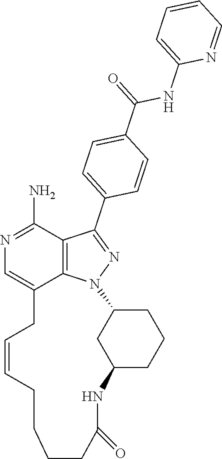 [SMILES: Nc1ncc2c3c1c(-c1ccc(C(=O)Nc4ccccn4)cc1)nn3[C@@H]1CCC[C@H](C1)NC(=O)CCCC/C=C\C2] | 7.10 | 549.28522 | 550.5 | 3 mg | 3% |
| 101a | H | Commerc | 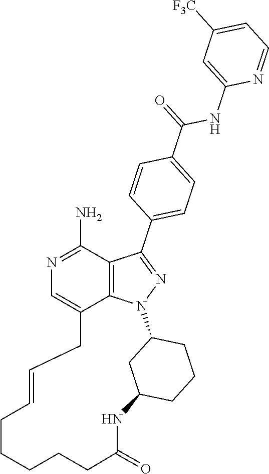 [SMILES: Nc1ncc2c3c1c(-c1ccc(C(=O)Nc4cc(C(F)(F)F)ccn4)cc1)nn3[C@@H]1CCC[C@H](C1)NC(=O)CCCCC/C=C/C2] | 9.55 | 631.28826 | 632.5 | 9 mg | 27.3% |
| 101b | H | Commerc | 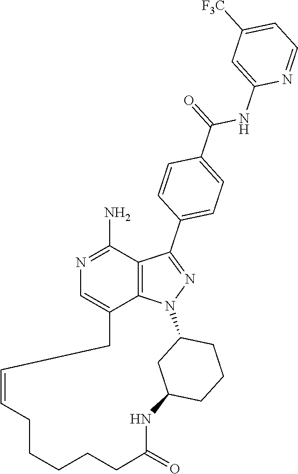 [SMILES: Nc1ncc2c3c1c(-c1ccc(C(=O)Nc4cc(C(F)(F)F)ccn4)cc1)nn3[C@@H]1CCC[C@H](C1)NC(=O)CCCCC/C=C\C2] | 10.04 | 631.28826 | 632.5 | 6 mg | 18.6% |
| 102a | H | Commerc | 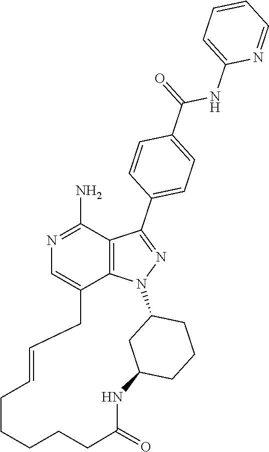 [SMILES: Nc1ncc2c3c1c(-c1ccc(C(=O)Nc4ccccn4)cc1)nn3[C@@H]1CCC[C@H](C1)NC(=O)CCCCC/C=C/C2] | 6.72 | 563.30087 | 564.5 | 6 mg | 13% |
| 102b | H | Commerc | 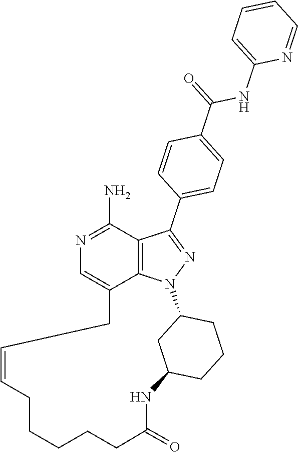 [SMILES: Nc1ncc2c3c1c(-c1ccc(C(=O)Nc4ccccn4)cc1)nn3[C@@H]1CCC[C@H](C1)NC(=O)CCCCC/C=C\C2] | 7.38 | 563.30087 | 564.5 | 2 mg | 4% |
| 103a | H | Commerc | 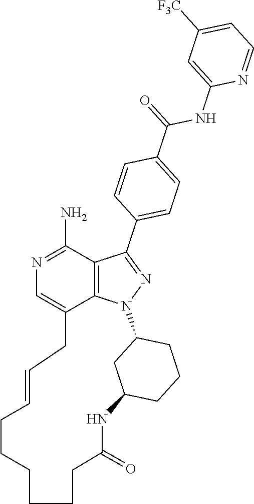 [SMILES: Nc1ncc2c3c1c(-c1ccc(C(=O)Nc4cc(C(F)(F)F)ccn4)cc1)nn3[C@@H]1CCC[C@H](C1)NC(=O)CCCCCC/C=C/C2] | 10.20 | 645.30391 | 646.5 | 6 mg | 13.4% |
| 103b | H | Commerc | 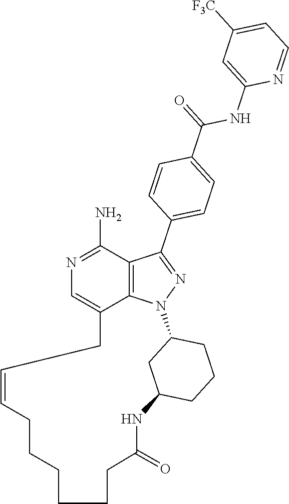 [SMILES: Nc1ncc2c3c1c(-c1ccc(C(=O)Nc4cc(C(F)(F)F)ccn4)cc1)nn3[C@@H]1CCC[C@H](C1)NC(=O)CCCCCC/C=C\C2] | 10.47 | 645.30391 | 646.5 | 2 mg | 4.5% |
| 104a | H | Commerc | 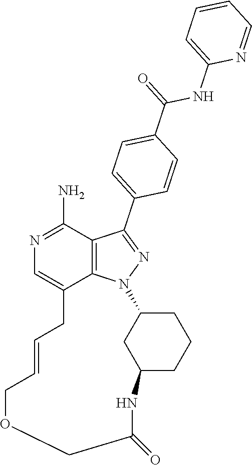 [SMILES: Nc1ncc2c3c1c(-c1ccc(C(=O)Nc4ccccn4)cc1)nn3[C@@H]1CCC[C@H](C1)NC(=O)COC/C=C/C2] | 5.97 | 537.24884 | 538.4 | 6 mg | 29% |
| 104b | H | Commerc | 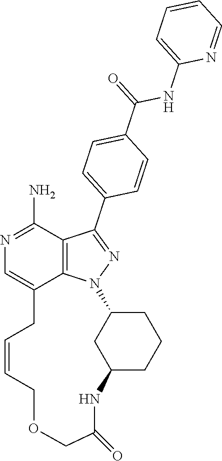 [SMILES: Nc1ncc2c3c1c(-c1ccc(C(=O)Nc4ccccn4)cc1)nn3[C@@H]1CCC[C@H](C1)NC(=O)COC/C=C\C2] | 6.55 | 537.24884 | 538.4 | 1 mg | 5% |
| 105 | H | Commerc | 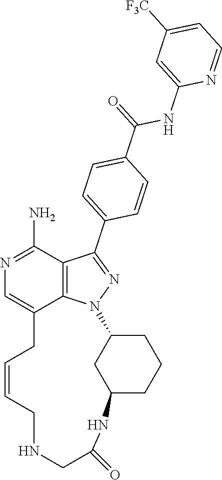 [SMILES: Nc1ncc2c3c1c(-c1ccc(C(=O)Nc4cc(C(F)(F)F)ccn4)cc1)nn3[C@@H]1CCC[C@H](C1)NC(=O)CNC/C=C\C2] | 6.86 | 604.25221 | 605.4 | 18 mg | 20% |
| 106 | H | Commerc | 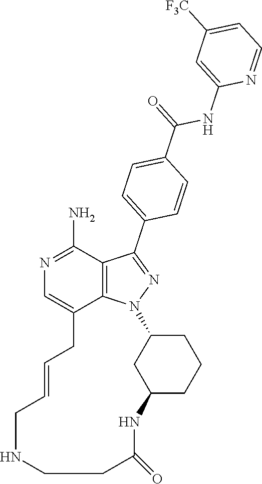 [SMILES: Nc1ncc2c3c1c(-c1ccc(C(=O)Nc4cc(C(F)(F)F)ccn4)cc1)nn3[C@@H]1CCC[C@H](C1)NC(=O)CCNC/C=C/C2] | 6.92 | 618.26786 | 619.4 | 35 mg | 44% |
| 107 | H | Commerc | 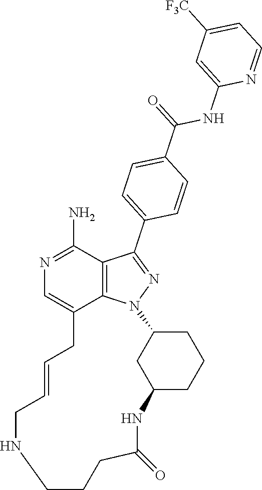 [SMILES: Nc1ncc2c3c1c(-c1ccc(C(=O)Nc4cc(C(F)(F)F)ccn4)cc1)nn3[C@@H]1CCC[C@H](C1)NC(=O)CCCNC/C=C/C2] | 6.84 | 632.28351 | 633.4 | 7 mg | 50% |
| 108 | H | Commerc | 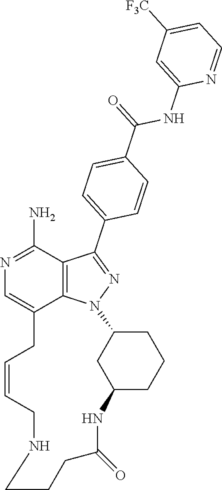 [SMILES: Nc1ncc2c3c1c(-c1ccc(C(=O)Nc4cc(C(F)(F)F)ccn4)cc1)nn3[C@@H]1CCC[C@H](C1)NC(=O)CCCNC/C=C\C2] | 7.35 | 632.28351 | 633.4 | 1.8 mg | 57% |
| 109a | H | Commerc | 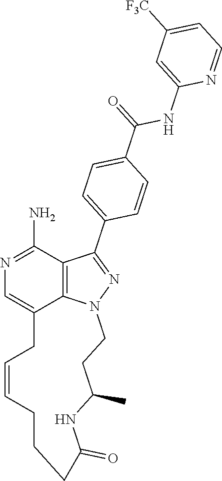 [SMILES: C[C@@H]1CCn2nc(-c3ccc(C(=O)Nc4cc(C(F)(F)F)ccn4)cc3)c3c(N)ncc(c32)C/C=C\CCCC(=O)N1] | 8.26 | 577.24131 | 578.3 | 7 mg | 7% |
| 109b | H | Commerc | 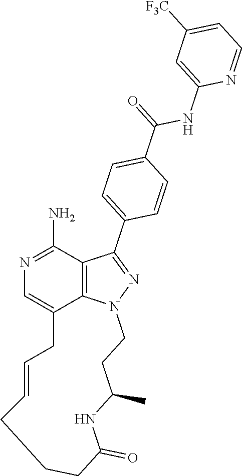 [SMILES: C[C@@H]1CCn2nc(-c3ccc(C(=O)Nc4cc(C(F)(F)F)ccn4)cc3)c3c(N)ncc(c32)C/C=C/CCCC(=O)N1] | 8.56 | 577.24131 | 578.4 | 7 mg | 7% |
| 110 | H | Commerc | 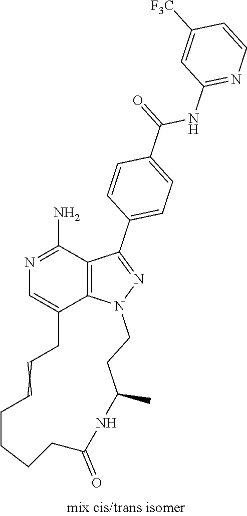 [SMILES: C[C@@H]1CCn2nc(-c3ccc(C(=O)Nc4cc(C(F)(F)F)ccn4)cc3)c3c(N)ncc(c32)CC=CCCCCC(=O)N1] | 8.75 | 591.25696 | 592.4 | 13 mg | 14.2% |
| 111 | H | Commerc | 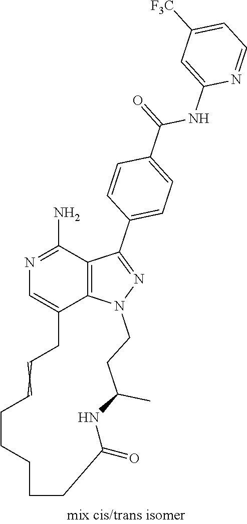 [SMILES: C[C@@H]1CCn2nc(-c3ccc(C(=O)Nc4cc(C(F)(F)F)ccn4)cc3)c3c(N)ncc(c32)CC=CCCCCCC(=O)N1] | 9.23 | 605.27261 | 606.4 | 2.5 mg | 3% |

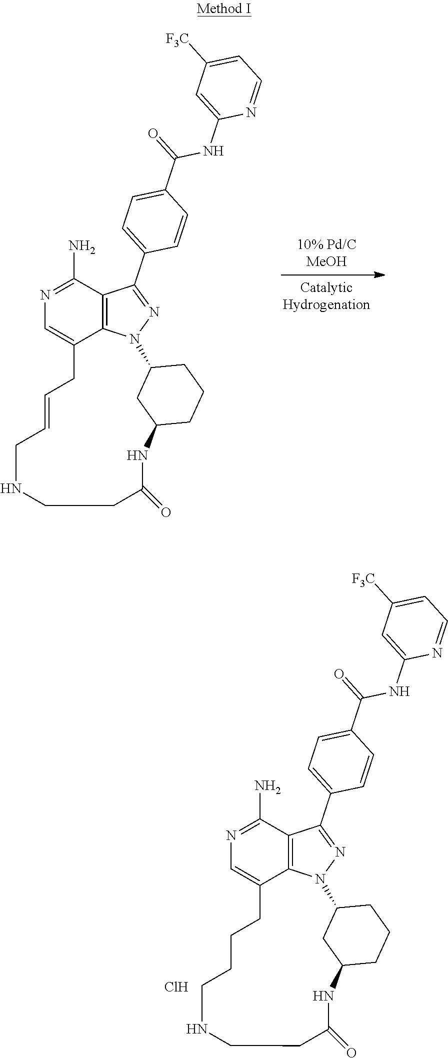 [SMILES: Nc1ncc2c3c1c(-c1ccc(C(=O)Nc4cc(C(F)(F)F)ccn4)cc1)nn3[C@@H]1CCC[C@H](C1)NC(=O)CCNC/C=C/C2 ; Cl ; Nc1ncc2c3c1c(-c1ccc(C(=O)Nc4cc(C(F)(F)F)ccn4)cc1)nn3[C@@H]1CCC[C@H](C1)NC(=O)CCNCCCC2]

### Example 112

To a solution of Example 106 (14 mg, 0.022 mmol) in methanol (2 mL) was added 44 μL 1N HCl-solution and 14 mg of 10% Pd/C. Catalytic hydrogenation was performed at room temperature for 16 h. The palladium-catalyst was filtered and the filtrate was concentrated in vacuo to give 14 mg of the title compound (Yield: quantitative). Data: LCMS (B) Rₜ: 9.23 min; m/z 604.5 [M+H]⁺.

The following Examples were synthesized following method I described for Example 112.

| | | | | | | Found | | |
|---|---|---|---|---|---|---|---|---|
| | | | | Rₜ (min) | | Mass | | |
| Example | Method | Intermediate | CHEMISTRY | (LCMS B) | Calc Mass | [M + H]⁺ | Amount | Yield |
| 112 | I | | 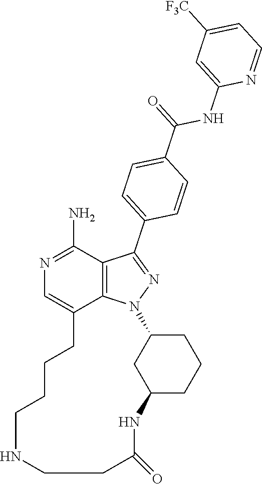 [SMILES: Nc1ncc2c3c1c(-c1ccc(C(=O)Nc4cc(C(F)(F)F)ccn4)cc1)nn3[C@@H]1CCC[C@H](C1)NC(=O)CCNCCCC2] | 6.97 | 620.28351 | 621.5 | 14 mg | 100% |
| 113 | I | | 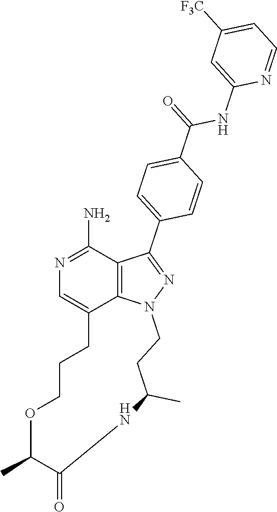 [SMILES: C[C@@H]1CCn2nc(-c3ccc(C(=O)Nc4cc(C(F)(F)F)ccn4)cc3)c3c(N)ncc(c32)CCCO[C@H](C)C(=O)N1] | 8.33 | 581.23622 | 582.5 | 2.5 mg | 20% |
| 114 | I | | 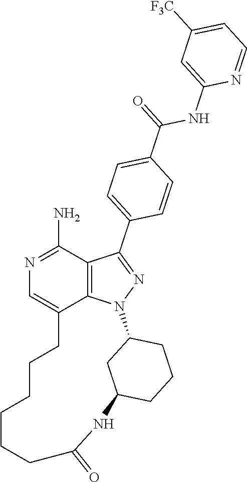 [SMILES: Nc1ncc2c3c1c(-c1ccc(C(=O)Nc4cc(C(F)(F)F)ccn4)cc1)nn3[C@@H]1CCC[C@H](C1)NC(=O)CCCCCC2] | 8.90 | 605.27261 | 606.4 | 3.4 mg | 28% |
| 115 | I | | 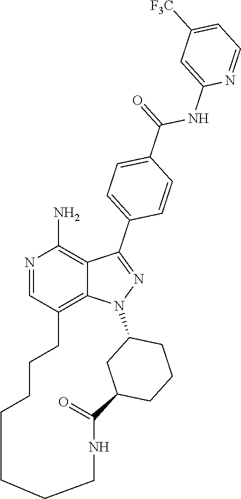 [SMILES: Nc1ncc2c3c1c(-c1ccc(C(=O)Nc4cc(C(F)(F)F)ccn4)cc1)nn3[C@@H]1CCC[C@H](C1)C(=O)NCCCCCCC2] | 10.17 | 619.28826 | 620.5 | 11 mg | 85% |
| 116 | I | | 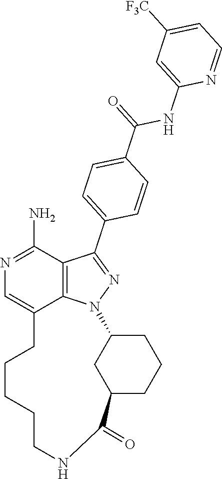 [SMILES: Nc1ncc2c3c1c(-c1ccc(C(=O)Nc4cc(C(F)(F)F)ccn4)cc1)nn3[C@@H]1CCC[C@H](C1)C(=O)NCCCCC2] | 9.27 | 591.25696 | 592.4 | 7 mg | 60% |
| 117 | I | | 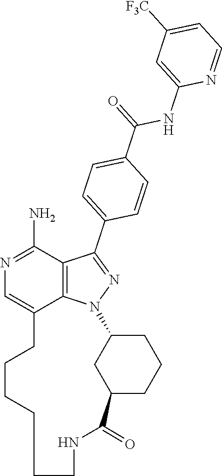 [SMILES: Nc1ncc2c3c1c(-c1ccc(C(=O)Nc4cc(C(F)(F)F)ccn4)cc1)nn3[C@@H]1CCC[C@H](C1)C(=O)NCCCCCC2] | 9.56 | 605.27261 | 606.5 | 2.5 mg | 31% |
| 118 | I | | 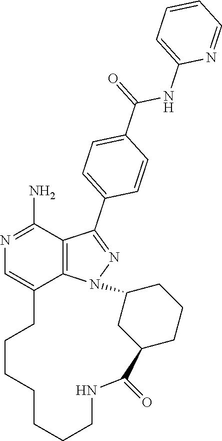 [SMILES: Nc1ncc2c3c1c(-c1ccc(C(=O)Nc4ccccn4)cc1)nn3[C@@H]1CCC[C@H](C1)C(=O)NCCCCCCC2] | 7.78 | 551.30087 | 552.5 | 4 mg | 52% |
| 119 | I | | 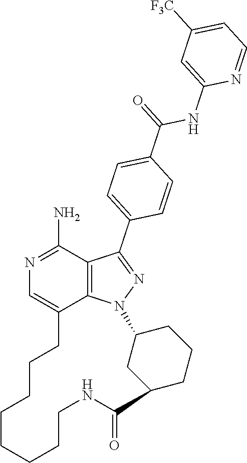 [SMILES: Nc1ncc2c3c1c(-c1ccc(C(=O)Nc4cc(C(F)(F)F)ccn4)cc1)nn3[C@@H]1CCC[C@H](C1)C(=O)NCCCCCCCC2] | 10.80 | 633.30391 | 634.5 | 3 mg | 68% |
| 120 | I | | 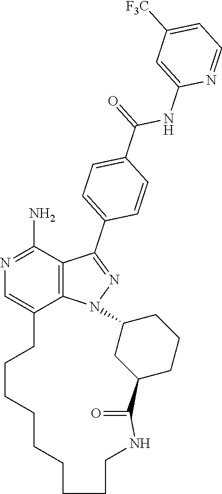 [SMILES: Nc1ncc2c3c1c(-c1ccc(C(=O)Nc4cc(C(F)(F)F)ccn4)cc1)nn3[C@@H]1CCC[C@H](C1)C(=O)NCCCCCCCCC2] | 11.85 | 647.31956 | 648.5 | 2 mg | 22% |
| 121 | I | | 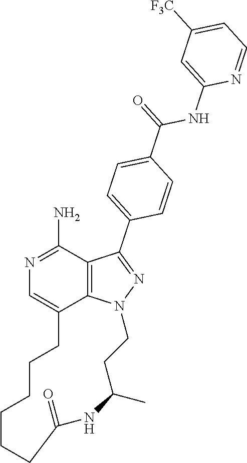 [SMILES: C[C@@H]1CCn2nc(-c3ccc(C(=O)Nc4cc(C(F)(F)F)ccn4)cc3)c3c(N)ncc(c32)CCCCCCC(=O)N1] | 8.60 | 579.25696 | 580.5 | 6.1 mg | 44% |
| 122 | I | | 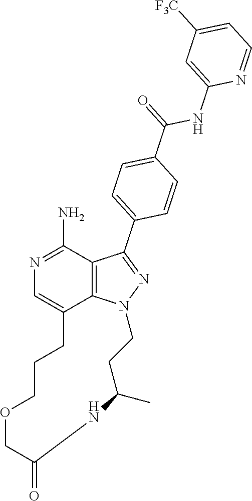 [SMILES: C[C@@H]1CCn2nc(-c3ccc(C(=O)Nc4cc(C(F)(F)F)ccn4)cc3)c3c(N)ncc(c32)CCCOCC(=O)N1] | 8.05 | 567.22057 | 568.4 | 4.7 mg | 33% |
| 123 | I | | 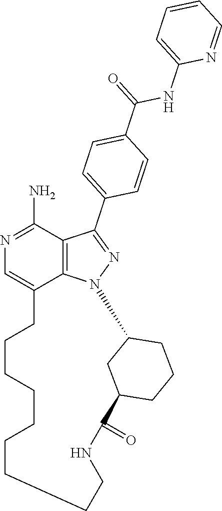 [SMILES: Nc1ncc2c3c1c(-c1ccc(C(=O)Nc4ccccn4)cc1)nn3[C@@H]1CCC[C@H](C1)C(=O)NCCCCCCCCC2] | 9.83 | 579.33217 | 580.5 | 6 mg | 61% |
| 124 | I | | 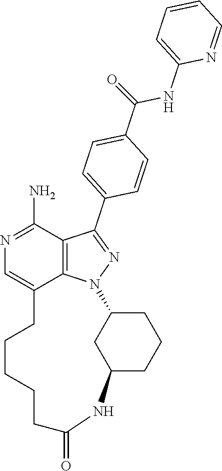 [SMILES: Nc1ncc2c3c1c(-c1ccc(C(=O)Nc4ccccn4)cc1)nn3[C@@H]1CCC[C@H](C1)NC(=O)CCCCC2] | 6.25 | 523.26957 | 524.5 | 6 mg | 32% |
| 125 | I | | 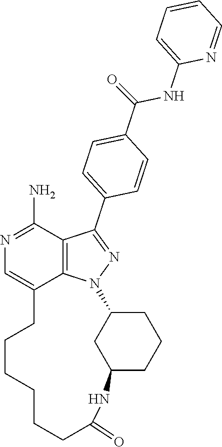 [SMILES: Nc1ncc2c3c1c(-c1ccc(C(=O)Nc4ccccn4)cc1)nn3[C@@H]1CCC[C@H](C1)NC(=O)CCCCCC2] | 6.54 | 537.28522 | 538.5 | 16 mg | 62% |
| 126 | I | | 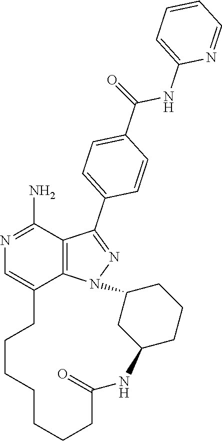 [SMILES: Nc1ncc2c3c1c(-c1ccc(C(=O)Nc4ccccn4)cc1)nn3[C@@H]1CCC[C@H](C1)NC(=O)CCCCCCC2] | 7.02 | 551.30087 | 552.5 | 6 mg | 35% |
| 127 | I | | 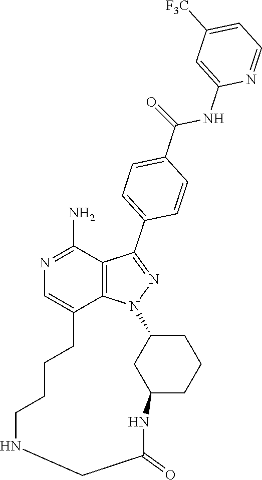 [SMILES: Nc1ncc2c3c1c(-c1ccc(C(=O)Nc4cc(C(F)(F)F)ccn4)cc1)nn3[C@@H]1CCC[C@H](C1)NC(=O)CNCCCC2] | 6.93 | 606.26786 | 607.4 | 5 mg | 29% |
| 128 | I | | 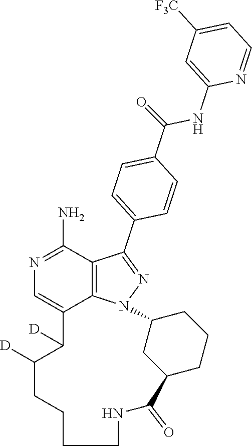 [SMILES: [2H]C1CCCCNC(=O)[C@@H]2CCC[C@H](C2)n2nc(-c3ccc(C(=O)Nc4cc(C(F)(F)F)ccn4)cc3)c3c(N)ncc(c32)C1[2H]] | 9.53 | 607.28516 | 608.4 | 10 mg | 100% |

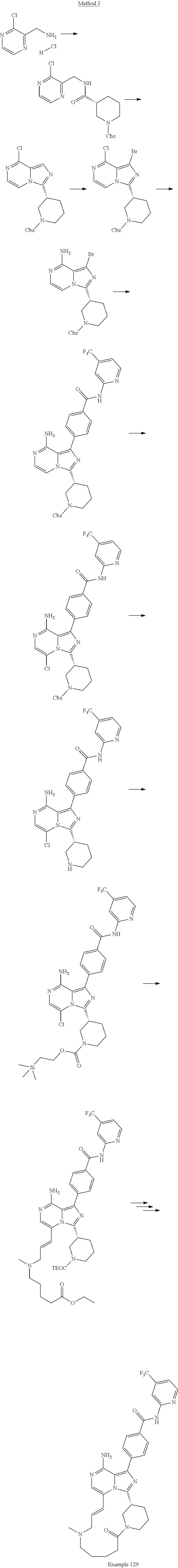

### Example 129

### (a) Benzyl(3R)-3-[(3-chloropyrazin-2-yl)methylcarbamoyl]piperidine-1-carboxylate

To a solution of (R)-piperidine-1,3-dicarboxylic acid 1-benzyl ester (8.3 g, 31.6 mmol) in dichloromethane (143 mL) was added subsequently, triethylamine (15.4 mL, 110 mmol) and HATU (12.0 g, 31.6 mmol). The resulting suspension was stirred at room temperature for 1 h. after which (3-chloropyrazin-2-yl)methanamine hydrochloride (7.68 g, 42.7 mmol) was added. The resulting mixture was stirred at room temperature o/n. The reaction mixture was filtered over a Buechner filter. The filtrate was washed with 5% aq. NaHCO₃-solution (150 mL), 5% aq. citric acid solution (150 mL), water (150 mL), dried over sodium sulfate filtered and concentrated under reduced pressure to give 14.5 g of the title compound (quant. crude yield). The product was used directly in the next step.

### (b) Benzyl(3R)-3-(8-chloroimidazo[1,5-a]pyrazin-3-yl)piperidine-1-carboxylate

Benzyl(3R)-3-[(3-chloropyrazin-2-yl)methylcarbamoyl]piperidine-1-carboxylate (14.5 g, 31.6 mmol) was dissolved in acetonitrile (130 mL), phosphorus oxychloride (14.7 mL, 158 mmol) was added and the mixture was stirred for 7 h. at 80° C. and at room temperature o/n. The mixture was added carefully to 25% ammonia (400 mL) and crushed ice (700 mL) keeping the temperature below 0° C. Ethyl acetate (400 mL) was added and the resulting mixture was stirred for 30 min. The water layer was separated and extracted with ethyl acetate. The combined organic layers were washed with water, brine, dried over sodium sulfate and concentrated in vacuo to give 9 g of the title compound (yield: 77%).

### (c) Benzyl(3R)-3-(1-bromo-8-chloro-imidazo[1,5-a]pyrazin-3-yl)piperidine-1-carboxylate

N-Bromosuccinimide (4.74 g, 26.6 mmol) was added to a cold (0° C.) solution of benzyl(3R)-3-(8-chloroimidazo[1,5-a]pyrazin-3-yl)piperidine-1-carboxylate (9 g, 24.2 mmol) in DMF (98 mL). The reaction mixture was stirred for 3 h. at 0° C. The mixture was quenched with 10% aq. Na₂S₂O₄-solution/5% aq. NaHCO₃-solution/brine and ethyl acetate. The phases were separated and the water layer was extracted with ethyl acetate. The combined organic phases were washed with water, brine, dried over sodium sulfate, filtered and concentrated under reduced pressure. The crude product was purified by column chromatography using SiO₂ and heptane/ethyl acetate=1/1 to 1/4 v/v %. All fractions containing the title compound were collected and concentrated in vacuo to give 9.4 g of the title compound (Yield: 86%).

### (d) Benzyl(3R)-3-(8-amino-1-bromo-imidazo[1,5-a]pyrazin-3-yl)piperidine-1-carboxylate

Benzyl(3R)-3-(1-bromo-8-chloro-imidazo[1,5-a]pyrazin-3-yl)piperidine-1-carboxylate (1 g, 2.22 mmol) was suspended in 25% ammonia (6 mL, 40 mmol) and placed in a sealed tube. The mixture was stirred at 120° C. o/n. After cooling, the mixture was concentrated in vacuo, dissolved in ethyl acetate and washed with water, brine, dried over sodium sulfate, filtered and concentrated under reduced pressure to give 860 mg (90%) of the title compound.

### (e) Benzyl(3R)-3-[8-amino-1-[4-[[4-(trifluoromethyl)-2-pyridyl]carbamoyl]phenyl]imidazo[1,5-alpyrazin-3-yl]piperidine-1-carboxylate

Benzyl(3R)-3-(8-amino-1-bromo-imidazo[1,5-a]pyrazin-3-yl)piperidine-1-carboxylate (860 mg, 2.0 mmol) was dissolved in dioxane/water=4/1 v/v % (25 mL) and potassium carbonate (1.38 g, 10.0 mmol) was added. The solution was purged with nitrogen for 5 min and 4-(4,4,5,5-tetramethyl-1,3,2-dioxaborolan-2-yl)-N-[4-(trifluoromethyl)-2-pyridyl]benzamide (Intermediate BP1) (788 mg, 2.0 mmol) and Pd(dppf)Cl₂.CH₂Cl₂ (41 mg, 0.05 mmol) were added. The reaction mixture was stirred for 4 h at 90° C. The reaction mixture was diluted with ethyl acetate after cooling to room temperature and filtered over Decalite™. The filtrate was washed with water, 0.2M NaOH-solution, water and brine, dried over sodium sulfate, filtered and concentrated under reduced pressure. The crude product was purified by column chromatography using SiO₂ and heptane/ethyl acetate=1/1 to 0/10 v/v %. All fractions containing the title compound were collected and concentrated in vacuo to give 1.0 g of the title compound (Yield: 81%).

### (f) Benzyl(3R)-3-[8-amino-5-chloro-1-[4-[[4-(trifluoromethyl)-2-pyridyl]carbamoyl]phenyl]-imidazo[1,5-a]pyrazin-3-yl]piperidine-1-carboxylate

To a solution of benzyl(3R)-3-[8-amino-1-[4-[[4-(trifluoromethyl)-2-pyridyl]carbamoyl]-phenyl]imidazo[1,5-a]pyrazin-3-yl]piperidine-1-carboxylate (1.00 g, 1.62 mmol) in acetic acid (6 mL) was added N-chlorosuccinimide (217 mg, 1.62 mmol) and the mixture was stirred at 80° C. for 1 h. After cooling to room temperature, the mixture was added dropwise to a stirred mixture of water (240 mL) and ethyl acetate (60 mL) and stirred 30 minutes at room temperature. The layers were separated and the water layer was extracted with ethyl acetate (2×60 mL). The combined organic layers were washed with water (300 mL), aq. Na₂CO₃-solution (10.6 g, 100 mmol in 50 mL water), brine (25 mL), dried over sodium sulfate, filtered and concentrated under reduced pressure. The crude product was purified by column chromatography using SiO₂ and heptane/ethyl acetate=1/1 to 0/10 v/v %. All fractions containing the title compound were collected and concentrated in vacuo to give 640 mg of the title compound (Yield: 61%).

### (g) 4-[8-Amino-5-chloro-3-[(3R)-3-piperidyl]imidazo[1,5-a]pyrazin-1-yl]-N-[4-(trifluoromethyl)-2-pyridyl]benzamide

To benzyl(3R)-3-[8-amino-5-chloro-1-[4-[[4-(trifluoromethyl)-2-pyridyl]carbamoyl]-phenyl]imidazo[1,5-a]pyrazin-3-yl]piperidine-1-carboxylate (640 mg, 0.98 mmol) was added 33% HBr/acetic acid solution (5.9 mL, 34 mmol) and the mixture was left at room temperature for 4 h. The mixture was diluted with water/brine (80 mL) and extracted with dichloromethane. The aqueous phase was neutralized with 1N NaOH-solution (60 mL), and then extracted with dichloromethane/methanol=9/1 v/v %. The organic layer was separated over a PE-filter. The filtrate was concentrated in vacuo to afford 340 mg of the title compound (yield: 67%).

### (h) 2-Trimethylsilylethyl(3R)-3-[8-amino-5-chloro-1-[4-[[4-(trifluoromethyl)-2-pyridyl]carbamoyl]-phenyl]imidazo[1,5-a]pyrazin-3-yl]piperidine-1-carboxylate

DiPEA (234 μL, 1.32 mmol) was added to a solution of 4-[8-amino-5-chloro-3-[(3R)-3-piperidyl]imidazo[1,5-a]pyrazin-1-yl]-N-[4-(trifluoromethyl)-2-pyridyl]benzamide (340 mg, 0.66 mmol) in dichloromethane (6.6 mL). Next, 1-[(2-trimethylsilyl)ethoxycarbonyloxy]pyrrolidin-2,5-dione (188 mg, 0.73 mmol) was added and the reaction mixture was stirred at room temperature o/n. The mixture was diluted with dichloromethane (30 mL) and water (30 mL0 was added and the mixture stirred for 30 minutes at room temperature. The aqueous layer was separated and extracted with dichloromethane. The combined organic layers were washed with water (25 mL), filtered over a PE filter and concentrated in vacuo. The crude product was purified by column chromatography using SiO₂ and ethyl acetate. All fractions containing the title compound were collected and concentrated in vacuo to give 320 mg of the title compound (Yield: 73%).

### (i) 2-Trimethylsilylethyl(3R)-3-[8-amino-5-[(E)-3-[(5-ethoxy-5-oxo-pentyl)-methyl-amino]prop-1-enyl]-1-[4-[[4-(trifluoromethyl)-2-pyridyl]carbamoyl]phenyl]imidazo[1,5-a]pyrazin-3-yl]piperidine-1-carboxylate

A mixture of 2-trimethylsilylethyl(3R)-3-[8-amino-5-chloro-1-[4-[[4-(trifluoromethyl)-2-pyridyl]carbamoyl]-phenyl]imidazo[1,5-a]pyrazin-3-yl]piperidine-1-carboxylate (160 mg, 0.24 mmol), ethyl 5-[methyl-[(E)-3-(4,4,5,5-tetramethyl-1,3,2-dioxaborolan-2-yl)allyl]amino]pentano-ate (Intermediate L19) (157 mg, 0.48 mmol) and potassium carbonate (99.4 mg, 0.72 mmol) in dioxane/water=4/1 v/v % (4.5 mL) was degassed with nitrogen for 5 min at 30° C. CataCXium® A Pd G3 (11 mg, 0.015 mmol) was added and the mixture was again degassed with nitrogen for 5 min at 30° C. The reaction mixture was stirred at 100° C. for 60 min under microwave irradiation. After cooling ethyl acetate was added and the mixture was stirred for 5 min. The mixture was filtered over Decalite™ and the filtrate was washed with 5% citric acid solution, water and brine.

The organic layer was separated, dried over sodium sulfate, filtered and then concentrated under reduced pressure. The residue was purified by column chromatography (dichloromethane/methanol=10/0 to 9/1 v/v %) to give 40 mg of the title compound.

### (j) Example 129

This compound was prepared in an analogous manner as described in Method C steps f and g, using 2-trimethylsilylethyl(3R)-3-[8-amino-5-[(E)-3-[(5-ethoxy-5-oxo-pentyl)-methyl-amino]prop-1-enyl]-1-[4-[[4-(trifluoromethyl)-2-pyridyl]carbamoyl]phenyl]imidazo[1,5-a]pyrazin-3-yl]piperidine-1-carboxylate to afford crude Example 129. Purification was performed using preparative HPLC to afford the title compound (3 mg, 10%). Data: LCMS (B) Rₜ: 6.52 min; m/z 633.4 [M+H]⁺.

The following Examples were synthesized following method J described for Example 129.

| | | | | | | Found | | |
|---|---|---|---|---|---|---|---|---|
| | | | | Rₜ (min) | | Mass | | |
| Example | Method | Intermediate | CHEMISTRY | (LCMS B) | Calc Mass | [M + H]⁺ | Amount | Yield |
| 129 | J | L19 | 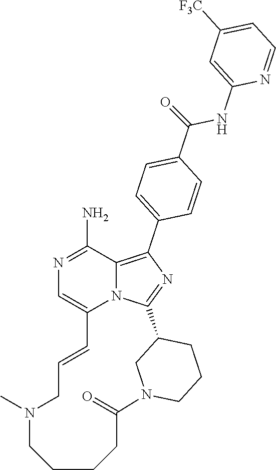 [SMILES: CN1C/C=C/c2cnc(N)c3c(-c4ccc(C(=O)Nc5cc(C(F)(F)F)ccn5)cc4)nc(n23)[C@@H]2CCCN(C2)C(=O)CCCC1] | 6.52 | 632.28351 | 633.4 | 3 mg | 10% |
| 130 | J | L22 | 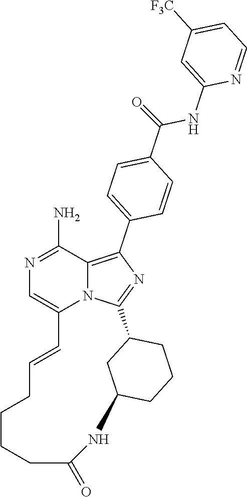 [SMILES: Nc1ncc2n3c(nc(-c4ccc(C(=O)Nc5cc(C(F)(F)F)ccn5)cc4)c13)[C@@H]1CCC[C@H](C1)NC(=O)CCCC/C=C/2] | 8.44 | 603.25696 | 604.4 | 4 mg | 38.3% |
| 131 | J | L15 | 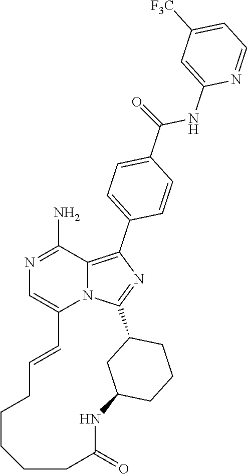 [SMILES: Nc1ncc2n3c(nc(-c4ccc(C(=O)Nc5cc(C(F)(F)F)ccn5)cc4)c13)[C@@H]1CCC[C@H](C1)NC(=O)CCCCC/C=C/2] | 8.54 | 617.27261 | 618.4 | 4 mg | 21.6% |
| 132 | J | L6 | 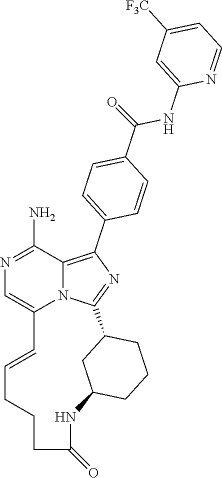 [SMILES: Nc1ncc2n3c(nc(-c4ccc(C(=O)Nc5cc(C(F)(F)F)ccn5)cc4)c13)[C@@H]1CCC[C@H](C1)NC(=O)CCC/C=C/2] | 8.28 | 589.24131 | 590.4 | 4 mg | 33.3% |

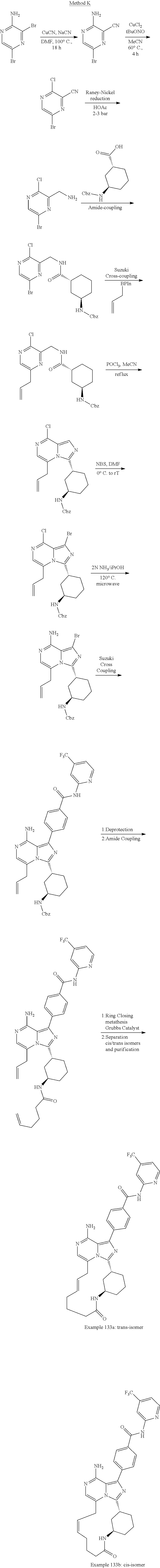

### Example 133

### (a) (6-Bromo-3-chloro-pyrazin-2-yl)methanamine hydrochloride

(6-bromo-3-chloro-pyrazin-2-yl)methanamine hydrochloride was prepared according to procedures described in WO 2013/010380 A1.

### (b) Benzyl N-[(1R,3R)-3-[(6-bromo-3-chloro-pyrazin-2-yl)methylcarbamoyl]cyclohexyl]-carbamate

To a solution of (1R,3R)-3-(benzyloxycarbonylamino)cyclohexanecarboxylic acid (Intermediate RP5) (800 mg, 2.88 mmol) in dichloromethane (15 mL) was added subsequently, triethylamine (1.2 mL, 8.65 mmol) and HATU (1.1 g, 2.88 mmol). The resulting suspension was stirred at room temperature for 1 h. after which (6-bromo-3-chloro-pyrazin-2-yl)methanamine hydrochloride (747 mg, 2.88 mmol) was added. The resulting mixture was stirred at room temperature o/n. The reaction mixture was filtered over a Buechner filter. The filtrate was washed with 5% aq. NaHCO₃-solution, 5% aq. citric acid solution, water, dried over sodium sulfate filtered and concentrated under reduced pressure to give 1.73 g of the title compound (quant. crude yield). The product was used directly in the next step.

### (c) Benzyl N-[(1R,3R)-3-[(6-allyl-3-chloro-pyrazin-2-yl)methylcarbamoyl]cyclohexyl]carbamate

A mixture of benzyl N-[(1R,3R)-3-[(6-bromo-3-chloro-pyrazin-2-yl)methylcarbamoyl]-cyclohexyl]carbamate (1.73 g, 3.33 mmol), 2-allyl-4,4,5,5-tetramethyl-1,3,2-dioxaborolane (560 mg, 2.91 mmol) and cesium fluoride (1.52 g, 9.99 mmol) in dioxane (33 mL) was degassed with nitrogen. Pd(dppf)Cl₂.CH₂Cl₂ (135 mg, 0.17 mmol) was added the reaction mixture was stirred for 2 h at 100° C. After cooling ethyl acetate was added and the mixture was stirred for 5 min. The mixture was filtered over Decalite™ and the filtrate was washed with 5% citric acid solution, water and brine. The organic layer was separated, dried over sodium sulfate, filtered and then concentrated under reduced pressure to give 1.62 g of the title compound (yield: quantitative crude).

### (d) Benzyl N-[(1R,3R)-3-(5-allyl-1-bromo-8-chloro-imidazo[1,5-a]pyrazin-3-yl)cyclohexyl]carba-mate

This compound was prepared in an analogous manner as described in Method J steps b and c, using benzyl N-[(1R,3R)-3-[(6-allyl-3-chloro-pyrazin-2-yl)methylcarbamoyl]cyclo-hexyl]carbamate to afford 680 mg of benzyl N-[(1R,3R)-3-(5-allyl-1-bromo-8-chloro-imidazo-[1,5-a]pyrazin-3-yl)cyclohexyl]carbamate (Yield: 40%).

### (e) Benzyl N-[(1R,3R)-3-(5-allyl-8-amino-1-bromo-imidazo[1,5-a]pyrazin-3-yl)cyclohexyl]carba-mate

Benzyl N-[(1R,3R)-3-(5-allyl-1-bromo-8-chloro-imidazo[1,5-a]pyrazin-3-yl)cyclohexyl]-carbamate (680 mg, 1.35 mmol) was suspended in 2N ammonia/isopropanol (18 mL) and placed in a sealed tube. The mixture was stirred at 120° C. for 14 h under microwave irradiation. After cooling, the mixture was concentrated in vacuo, dissolved in ethyl acetate and washed with water, brine, dried over sodium sulfate, filtered and concentrated under reduced pressure The crude product was purified by column chromatography using SiO₂ and heptane/ethyl acetate=1/1 to 0/10 v/v %. All fractions containing the title compound were collected and concentrated in vacuo to give 515 mg of the title compound (Yield: 79%).

### Example 133

This compound was prepared in an analogous manner as described in Method J step e and g, Method H steps b-d using benzyl N-[(1R,3R)-3-(5-allyl-8-amino-1-bromo-imidazo[1,5-a]pyrazin-3-yl)cyclohexyl]carba-mate and 5-hexenoic acid to afford crude Example 133.

Purification was performed using preparative HPLC to afford the two separated trans and cis-isomers of the title compound. Example 133a is the first eluting isomer, which corresponds with the trans-isomer (5 mg, 9%) Data: LBMS (B) Rₜ:8.16 mn; m/z604.4 [M+H]. Example 133b is the last eluting isomer, which corresponds with the cis-isomer (5 mg, 9%) Data: LCMS (B) Rt 8.54 m[; m/z 604.4 μM+H]⁺.

The following Examples were synthesized following method K described for Example 133.

| | | | | | | Found | | |
|---|---|---|---|---|---|---|---|---|
| | | | | Rₜ (min) | | Mass | | |
| Example | Method | Intermediate | CHEMISTRY | (LCMS B) | Calc Mass | [M + H]⁺ | Amount | Yield |
| 133a | K | Commerc | 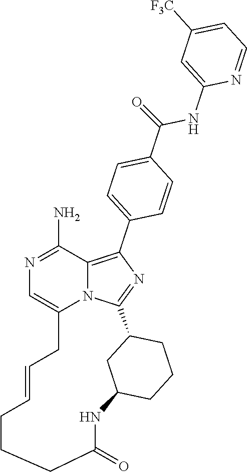 [SMILES: Nc1ncc2n3c(nc(-c4ccc(C(=O)Nc5cc(C(F)(F)F)ccn5)cc4)c13)[C@@H]1CCC[C@H](C1)NC(=O)CCC/C=C/C2] | 8.16 | 603.25696 | 604.4 | 5 mg | 9% |
| 133b | K | Commerc | 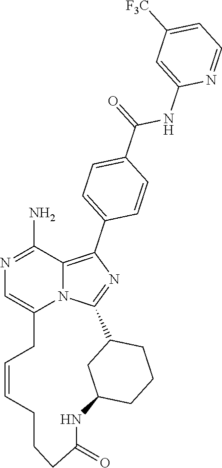 | 8.54 | 603.25696 | 604.4 | 5 mg | 9% |
| 134 | K | Commerc | 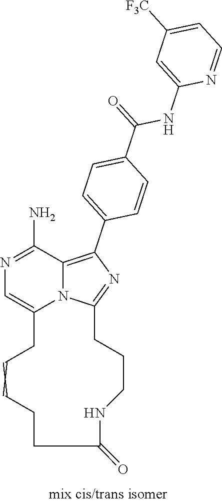 [SMILES: Nc1ncc2n3c(nc(-c4ccc(C(=O)Nc5cc(C(F)(F)F)ccn5)cc4)c13)CCCNC(=O)CCC=CC2] | 7.03/7.17 | 549.21001 | 550.3 | 1 mg | |
| 135 | K | Commerc | 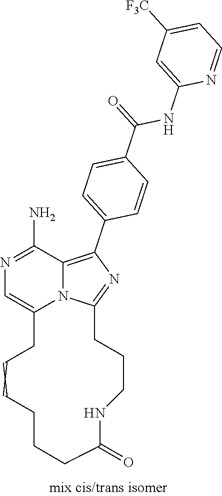 [SMILES: Nc1ncc2n3c(nc(-c4ccc(C(=O)Nc5cc(C(F)(F)F)ccn5)cc4)c13)CCCNC(=O)CCCC=CC2] | 7.27/7.65 | 563.22566 | 564.4 | 1 mg | |
| 136 | K | Commerc | 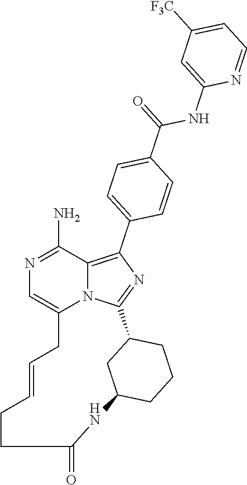 [SMILES: Nc1ncc2n3c(nc(-c4ccc(C(=O)Nc5cc(C(F)(F)F)ccn5)cc4)c13)[C@@H]1CCC[C@H](C1)NC(=O)CC/C=C/C2] | 7.71 | 589.24131 | 590.4 | 2 mg | |

The following Examples were synthesized following methods as depicted in the table.

| | | | | | | Found | | |
|---|---|---|---|---|---|---|---|---|
| | | | | Rₜ (min) | | Mass | | |
| Example | Method | Intermediate | CHEMISTRY | (LCMS B) | Calc Mass | [M + H]⁺ | Amount | Yield |
| 137 | B | L47 | 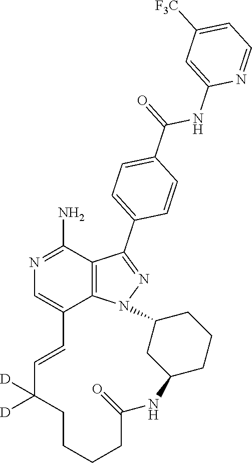 [SMILES: [2H]C1([2H])/C=C/c2cnc(N)c3c(-c4ccc(C(=O)Nc5cc(C(F)(F)F)ccn5)cc4)nn(c23)[C@@H]2CCC[C@H](C2)NC(=O)CCCC1] | 9.32 | 619.28516 | 620.4 | 7 mg | 24.3% |
| 138 | B | L48 | 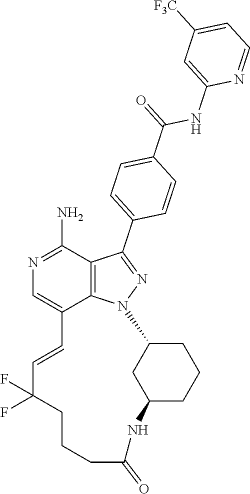 [SMILES: Nc1ncc2c3c1c(-c1ccc(C(=O)Nc4cc(C(F)(F)F)ccn4)cc1)nn3[C@@H]1CCC[C@H](C1)NC(=O)CCCC(F)(F)/C=C/2] | 9.55 | 639.23811 | 640.5 | 1 mg | |

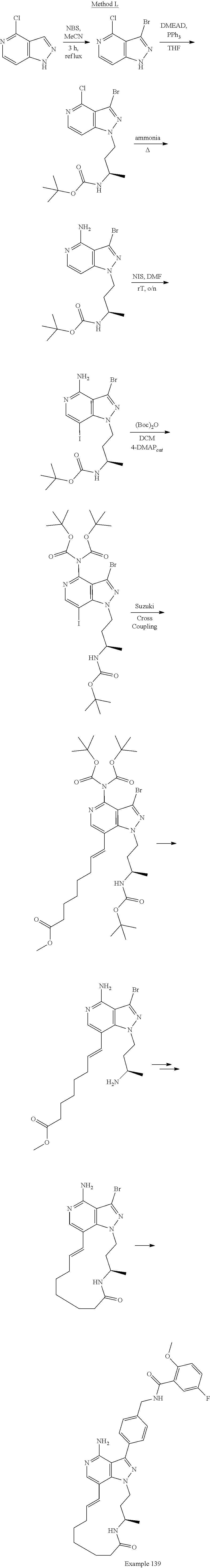

### Example 139

### (a) 3-Bromo-4-chloro-1 H-pyrazolo[4,3-c]pyridine

To a suspension of 4-chloro-1 H-pyrazolo[4,3-c]pyridine (1.61 g, 10.5 mmol) in acetonitrile (50 mL) was added N-bromosuccinimide (1.87 g, 10.5 mmol) and the reaction mixture was stirred under reflux for 3 h. After cooling, the mixture was stirred at room temperature o/n upon which a precipitate formed. The mixture was concentrated and the residue was stirred 1h at room temperature in water/ethanol=1/9 v/v % (20 mL). Next, additional water/ethanol=9/1 v/v % was added dropwise and stirring was continued 30 minutes at room temperature. The solids were filtered and the residue was dried in vacuo to give 1.3 g of 3-bromo-4-chloro-1 H-pyrazolo[4,3-c]pyridine (yield: 53%).

### (b) tert-Butyl N-[(1R)-3-(3-bromo-4-chloro-pyrazolo[4,3-c]pyridin-1-yl)-1-methyl-propyl]carba-mate

To an ice-cold (0° C.) solution of tert-butyl N-[(1R)-3-hydroxy-1-methyl-propyl]carba-mate (1.3 g, 6.86 mmol), 3-bromo-4-chloro-1 H-pyrazolo[4,3-c]pyridine (1.33 g, 5.72 mmol) and triphenylphosphine (1.80 g, 6.86 mmol) in THE (57 mL) was added dropwise a solution of di-2-methoxyethyl azodicarboxylate (1.61 g, 6.86 mmol) in THE (10 mL). The mixture was stirred for 30 min at 4° C. and then allowed to warm to room temperature and stirred o/n. To the mixture was added water/5% aq. citric acid solution/ethyl acetate=1/1/1 v/v % (150 mL). The resulting mixture was stirred for 30 min at room temperature. The layers were separated and the water layer was extracted with ethyl acetate (50 mL). The combined organic layers were washed with 5% aq. citric acid solution (2×75 mL), water (75 mL), brine (25 mL), dried (Na₂SO₄) filtered and concentrated under reduced pressure. The residue was purified by column chromatography using SiO₂ and heptane/ethyl acetate=95/5 to 0/10 v/v %. All fractions containing the title compound were collected and concentrated in vacuo to give 1.43 g of tert-butyl N-[(1R)-3-(3-bromo-4-chloro-pyrazolo[4,3-c]pyridin-1-yl)-1-methyl-propyl]carbamate (isomeric mixture in a 84/16 ratio) (yield: 62%).

### (c) tert-Butyl N-[(1R)-3-(4-amino-3-bromo-pyrazolo[4,3-c]pyridin-1-yl)-1-methyl-propyl]carba-mate

tert-Butyl N-[(1R)-3-(3-bromo-4-chloro-pyrazolo[4,3-c]pyridin-1-yl)-1-methyl-propyl]car-bamate (715 mg, 1.77 mmol) was suspended in 2N ammonia/isopropanol (9 mL) and 25% ammonia solution (9 mL) and placed in a sealed tube. The mixture was stirred at 120° C. for 20 h under microwave irradiation. After cooling, the mixture was concentrated in vacuo, dissolved in ethyl acetate and washed with water, brine, dried over sodium sulfate, filtered and concentrated under reduced pressure The crude product was purified by column chromatography using SiO₂ and heptane/ethyl acetate=95/5 to 0/10 v/v %. All fractions containing the title compound were collected and concentrated in vacuo to give 470 mg of the title compound (Yield: 35%).

### (d) tert-Butyl N-[(1R)-3-(4-amino-3-bromo-7-iodo-pyrazolo[4,3-c]pyridin-1-yl)-1-methyl-propyl]-carbamate

To a cold (0° C.) solution of tert-butyl N-[(1R)-3-(4-amino-3-bromo-pyrazolo[4,3-c]pyri-din-1-yl)-1-methyl-propyl]carbamate (220 mg, 0.57 mmol) in DMF (5.7 mL) was added N-iodosuccinimide (192 mg, 0.85 mmol). The reaction mixture was stirred o/n allowing the mixture to warm up to room temperature. The mixture was diluted with ethyl acetate and washed with sodium thiosulfate solution, water, brine, dried over sodium sulfate, filtered and concentrated in vacuo. The crude product was purified by column chromatography using SiO₂ and heptane/ethyl acetate=95/5 to 0/10 v/v %. All fractions containing the title compound were collected and concentrated in vacuo to give 250 mg of the title compound (Yield: 86%).

### (e) tert-Butyl N-[3-bromo-1-[(3R)-3-(tert-butoxycarbonylamino)butyl]-7-iodo-pyrazolo[4,3-c]pyridin-4-yl]-N-tert-butoxycarbonyl-carbamate

To a suspension of tert-butyl N-[(1R)-3-(4-amino-3-bromo-7-iodo-pyrazolo[4,3-c]pyri-din-1-yl)-1-methyl-propyl]carbamate (250 mg, 0.49 mmol) and 4-DMAP (1.5 mg, 0.01 mmol) in dichloromethane (4.9 mL) was added di-tert-butyl dicarbonate (160 mg, 0.74 mmol). The reaction mixture was stirred at room temperature o/n. Dichloromethane (10 mL) and sat. aq.

NaHCO₃-solution were added. The water layer was separated and extracted with dichloromethane (10 mL). The combined organic layers were washed with water (10 mL), 5% aq. citric acid solution (10 mL), water (10 mL), filtered over a PE filter and concentrated to give 340 mg tert-butyl N-[3-bromo-1-[(3R)-3-(tert-butoxycarbonylamino)butyl]-7-iodo-pyrazolo[4,3-c]pyridin-4-yl]-N-tert-butoxycarbonyl-carbamate (yield: 95%).

### (f) Methyl(E)-8-[4-[bis(tert-butoxycarbonyl)amino]-3-bromo-1-[(3R)-3-(tert-butoxycarbonyl-amino)butyl]pyrazolo[4,3-c]pyridin-7-yl]oct-7-enoate

N-[3-bromo-1-[(3R)-3-(tert-butoxycarbonylamino)butyl]-7-iodo-pyrazolo[4,3-c]pyridin-4-yl]-N-tert-butoxycarbonyl-carbamate (240 mg, 0.34 mmol) was dissolved in dioxane/water=4/1 v/v % (12 mL) and cesium carbonate (326 mg, 1.0 mmol) was added. The solution was purged with nitrogen for 5 min and methyl(E)-8-(4,4,5,5-tetramethyl-1,3,2-dioxaborolan-2-yl)oct-7-enoate (Intermediate L15) (120 mg, 0.43 mmol) and Pd(dppf)Cl₂.CH₂Cl₂ (29 mg, 0.034 mmol) were added. The reaction mixture was stirred for 4 h at 82° C. The reaction mixture was diluted with ethyl acetate after cooling to room temperature and filtered over Decalite™. The filtrate was washed with water and brine, dried over sodium sulfate, filtered and concentrated under reduced pressure. The crude product was purified by column chromatography using SiO₂ and heptane/ethyl acetate=95/5 to 0/10 v/v %. All fractions containing the title compound were collected and concentrated in vacuo to give 220 mg of the title compound (Yield: 69%).

### (g) Example 139

This compound was prepared in an analogous manner as described in Method C steps f and g, using methyl(E)-8-[4-[bis(tert-butoxycarbonyl)amino]-3-bromo-1-[(3R)-3-(tert-butoxycarbonyl-amino)butyl]pyrazolo[4,3-c]pyridin-7-yl]oct-7-enoate to afford crude Example 139. Purification was performed using preparative HPLC to afford the title compound (4 mg, 13.4%). Data: LCMS (B) Rₜ: 8.05 min; m/z 585.5 [M+H]⁺.

The following Examples were synthesized following methods as depicted in the table.

| | | | | Rₜ (min) | | Found Mass | | |
|---|---|---|---|---|---|---|---|---|
| Example | Method | Intermediate | CHEMISTRY | (LCMS B) | Calc Mass | [M + H]⁺ | Amount | Yield |
| 139 | L | L15 | 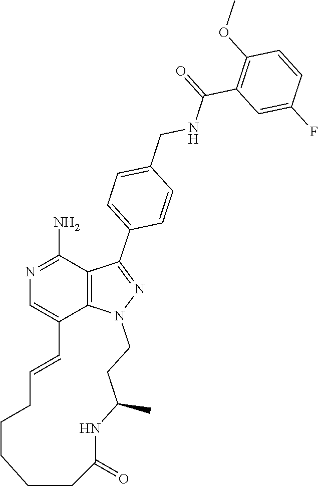 [SMILES: COc1ccc(F)cc1C(=O)NCc1ccc(-c2nn3c4c(cnc(N)c24)/C=C/CCCCCC(=O)N[C@H](C)CC3)cc1] | 8.05 | 584.29112 | 585.5 | 4 mg | 13.4% |
| 140 | I | | 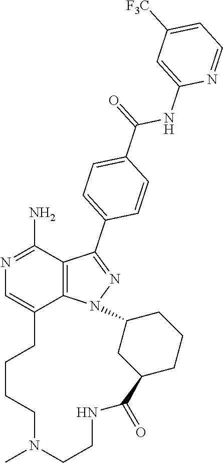 [SMILES: CN1CCCCc2cnc(N)c3c(-c4ccc(C(=O)Nc5cc(C(F)(F)F)ccn5)cc4)nn(c23)[C@@H]2CCC[C@H](C2)C(=O)NCC1] | 6.85 | 634.29916 | 635.5 | 5.5 mg | 54% |
| 141 | B | L49 | 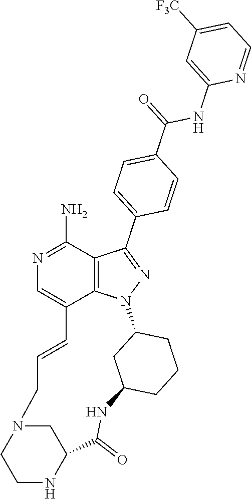 [SMILES: Nc1ncc2c3c1c(-c1ccc(C(=O)Nc4cc(C(F)(F)F)ccn4)cc1)nn3[C@@H]1CCC[C@H](C1)NC(=O)[C@H]1CN(C/C=C/2)CCN1] | 7.13 | 645.27876 | 646.5 | 16 mg | 67% |
| 142 | E | L49 | 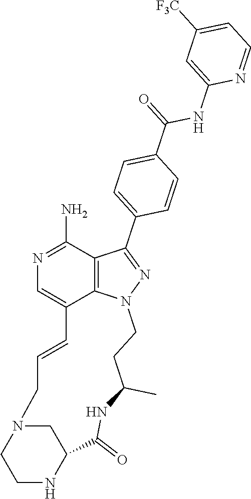 [SMILES: C[C@@H]1CCn2nc(-c3ccc(C(=O)Nc4cc(C(F)(F)F)ccn4)cc3)c3c(N)ncc(c32)/C=C/CN2CCN[C@H](C2)C(=O)N1] | 6.43 | 619.26311 | 620.5 | 7.3 mg | 84% |
| 143 | E | L50 | 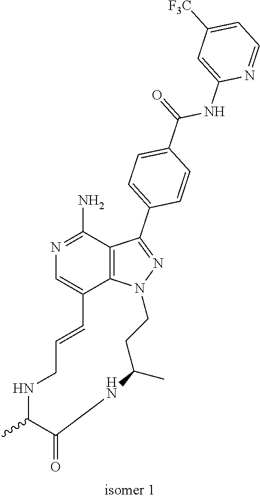 [SMILES: CC1NC/C=C/c2cnc(N)c3c(-c4ccc(C(=O)Nc5cc(C(F)(F)F)ccn5)cc4)nn(c23)CC[C@@H](C)NC1=O] | 6.59 | 578.23656 | 579.4 | 6.1 mg | 36% |
| 144 | E | L50 | 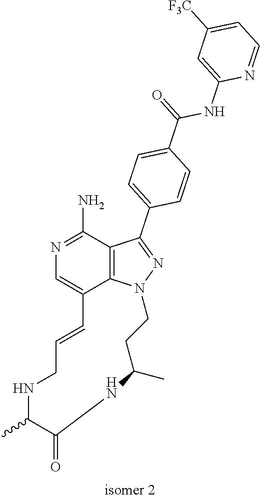 [SMILES: CC1NC/C=C/c2cnc(N)c3c(-c4ccc(C(=O)Nc5cc(C(F)(F)F)ccn5)cc4)nn(c23)CC[C@@H](C)NC1=O] | 6.47 | 578.23656 | 579.4 | 6.7 mg | 40% |
| 145 | B | L50 | 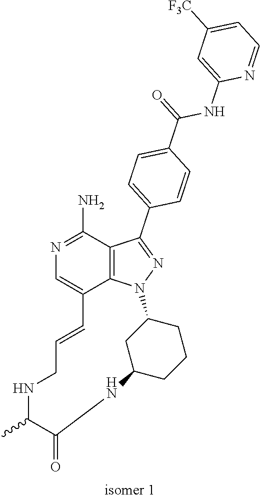 [SMILES: CC1NC/C=C/c2cnc(N)c3c(-c4ccc(C(=O)Nc5cc(C(F)(F)F)ccn5)cc4)nn(c23)[C@@H]2CCC[C@H](C2)NC1=O] | 7.15 | 604.25221 | 605.4 | 4.4 mg | 78% |
| 146 | B | L50 | 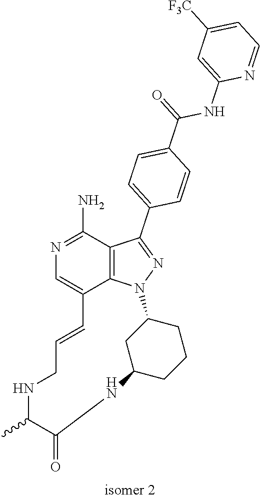 [SMILES: CC1NC/C=C/c2cnc(N)c3c(-c4ccc(C(=O)Nc5cc(C(F)(F)F)ccn5)cc4)nn(c23)[C@@H]2CCC[C@H](C2)NC1=O] | 7.05 | 604.25221 | 605.4 | 4.2 mg | 99% |
| 147 | B | L51 | 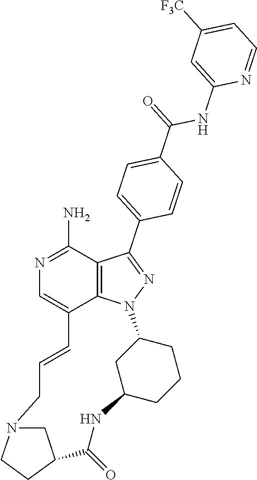 [SMILES: Nc1ncc2c3c1c(-c1ccc(C(=O)Nc4cc(C(F)(F)F)ccn4)cc1)nn3[C@@H]1CCC[C@H](C1)NC(=O)[C@@H]1CCN(C/C=C/2)C1] | 7.27 | 630.26786 | 631.4 | 16 mg | 69% |
| 148 | E | L51 | 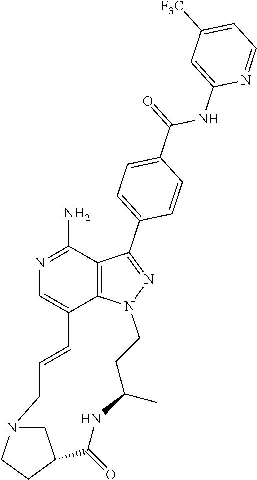 [SMILES: C[C@@H]1CCn2nc(-c3ccc(C(=O)Nc4cc(C(F)(F)F)ccn4)cc3)c3c(N)ncc(c32)/C=C/CN2CC[C@H](C2)C(=O)N1] | 6.74 | 604.25221 | 605.4 | 5.2 mg | 41% |
| 149 | A | L52 | 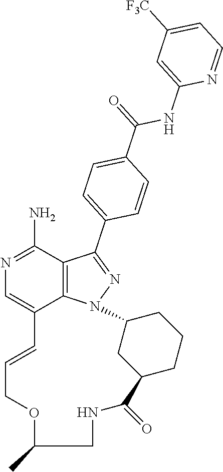 [SMILES: C[C@@H]1CNC(=O)[C@@H]2CCC[C@H](C2)n2nc(-c3ccc(C(=O)Nc4cc(C(F)(F)F)ccn4)cc3)c3c(N)ncc(c32)/C=C/CO1] | 9.27 | 619.25187 | 620.4 | 11 mg | 6% |
| 150 | B | L53 | 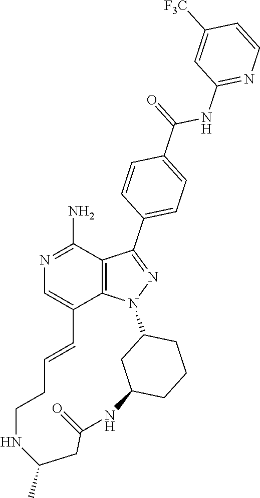 | 7.19 | 632.28351 | 633.5 | 41.6 mg | 80% |
| 151 | B | L54 | 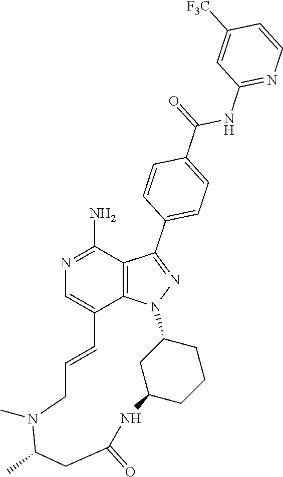 [SMILES: C[C@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CN1C] | 7.17 | 632.28351 | 633.4 | 8.5 mg | 58.4% |
| 152 | B | L55 | 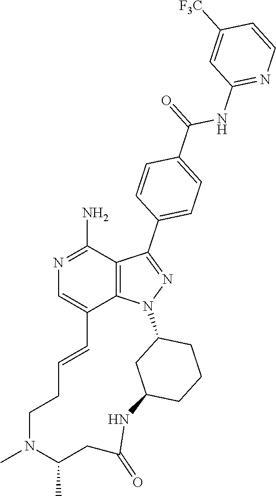 [SMILES: C[C@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCN1C] | 7.18 | 646.29916 | 647.5 | 28 mg | 93.2% |
| 153 | E | L24 | 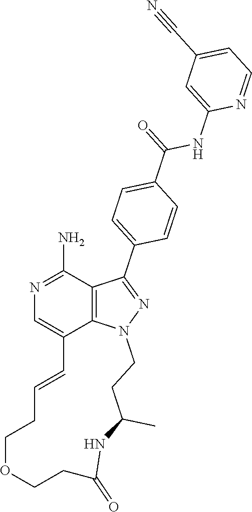 [SMILES: C[C@@H]1CCn2nc(-c3ccc(C(=O)Nc4cc(C#N)ccn4)cc3)c3c(N)ncc(c32)/C=C/CCOCCC(=O)N1] | 6.61 | 550.24409 | 551.3 | 31 mg | 67% |
| 154 | A | L56 | 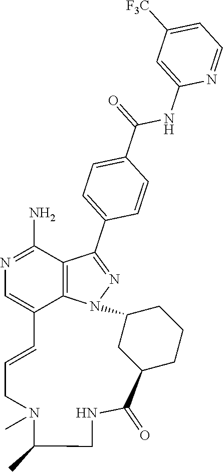 [SMILES: C[C@@H]1CNC(=O)[C@@H]2CCC[C@H](C2)n2nc(-c3ccc(C(=O)Nc4cc(C(F)(F)F)ccn4)cc3)c3c(N)ncc(c32)/C=C/CN1C] | 7.46 | 632.28351 | 633.5 | 6 mg | 6% |
| 155 | B | L24 | 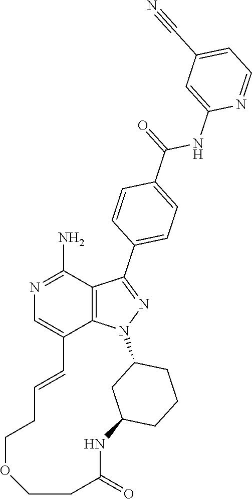 [SMILES: N#Cc1ccnc(NC(=O)c2ccc(-c3nn4c5c(cnc(N)c35)/C=C/CCOCCC(=O)N[C@@H]3CCC[C@@H]4C3)cc2)c1] | 7.50 | 576.25974 | 577.4 | 38 mg | 81% |
| 156 | B | L57 | 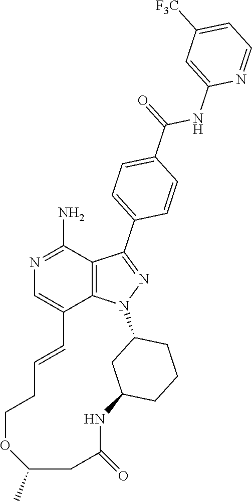 [SMILES: C[C@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCO1] | 4.02 (LCMS A) | 633.26752 | 634.4 | 12 mg | 75% |
| 157 | B | L57 | 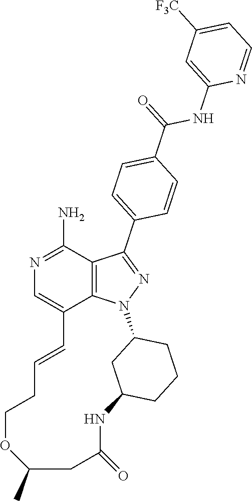 [SMILES: C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCO1] | 4.03 (LCMS A) | 633.26752 | 634.4 | 9 mg | 49% |
| 158 | E | L57 | 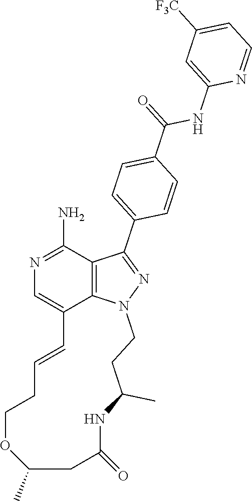 [SMILES: C[C@@H]1CCn2nc(-c3ccc(C(=O)Nc4cc(C(F)(F)F)ccn4)cc3)c3c(N)ncc(c32)/C=C/CCO[C@@H](C)CC(=O)N1] | 8.50 | 607.25187 | 608.4 | 8 mg | 61% |
| 159 | E | L57 | 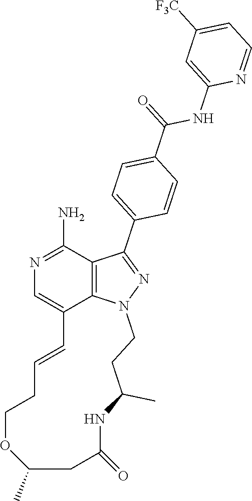 [SMILES: C[C@@H]1CCn2nc(-c3ccc(C(=O)Nc4cc(C(F)(F)F)ccn4)cc3)c3c(N)ncc(c32)/C=C/CCO[C@@H](C)CC(=O)N1] | 8.50 | 607.25187 | 608.4 | 11 mg | 62% |
| 160 | B | L58 | 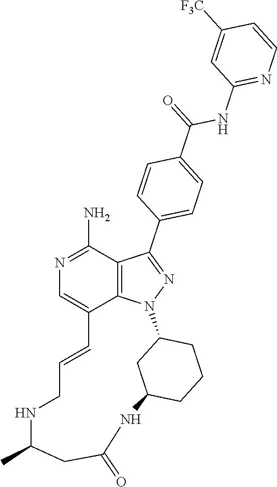 [SMILES: C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CN1] | 6.92 | 618.26786 | 619.4 | 33 mg | 92% |
| 161 | B | L57 | 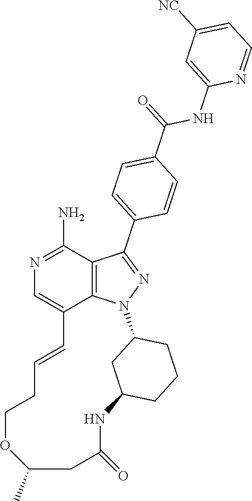 [SMILES: C[C@H]1CC(=O)N[C@@H]2CCC[C@H](C2)n2nc(-c3ccc(C(=O)Nc4cc(C#N)ccn4)cc3)c3c(N)ncc(c32)/C=C/CCO1] | 7.71 | 590.27539 | 591.4 | 6.1 mg | 26% |
| 162 | B | L57 | 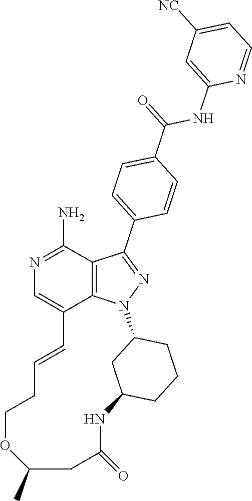 [SMILES: C[C@@H]1CC(=O)N[C@@H]2CCC[C@H](C2)n2nc(-c3ccc(C(=O)Nc4cc(C#N)ccn4)cc3)c3c(N)ncc(c32)/C=C/CCO1] | 7.80 | 590.27539 | 591.5 | 5.4 mg | 33% |
| 163 | C | L15 | 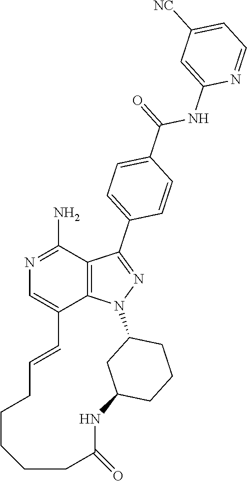 [SMILES: N#Cc1ccnc(NC(=O)c2ccc(-c3nn4c5c(cnc(N)c35)/C=C/CCCCCC(=O)N[C@@H]3CCC[C@@H]4C3)cc2)c1] | 7.80 | 574.28047 | 575.4 | 14 mg | 35% |
| 164 | C | L23 | 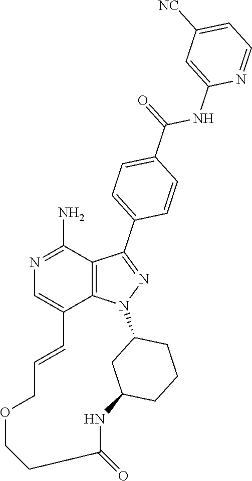 [SMILES: N#Cc1ccnc(NC(=O)c2ccc(-c3nn4c5c(cnc(N)c35)/C=C/COCCC(=O)N[C@@H]3CCC[C@@H]4C3)cc2)c1] | 3.57 (LCMS A) | 562.24409 | 563.4 | 5 mg | 13% |
| 165 | C | L31 | 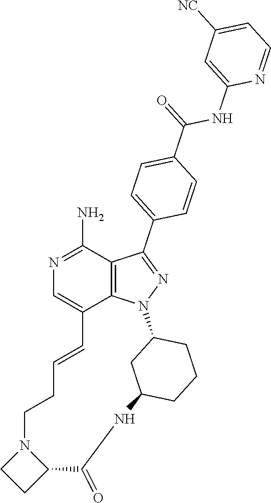 [SMILES: N#Cc1ccnc(NC(=O)c2ccc(-c3nn4c5c(cnc(N)c35)/C=C/CCN3CC[C@H]3C(=O)N[C@@H]3CCC[C@@H]4C3)cc2)c1] | 5.65 | 587.27572 | 588.4 | 9 mg | 32% |
| 166 | C | L24 | 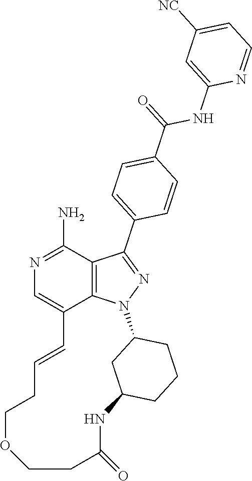 [SMILES: N#Cc1ccnc(NC(=O)c2ccc(-c3nn4c5c(cnc(N)c35)/C=C/CCOCCC(=O)N[C@@H]3CCC[C@@H]4C3)cc2)c1] | 7.56 | 576.25974 | 577.4 | 19 mg | 39% |
| 167 | B | L28 | 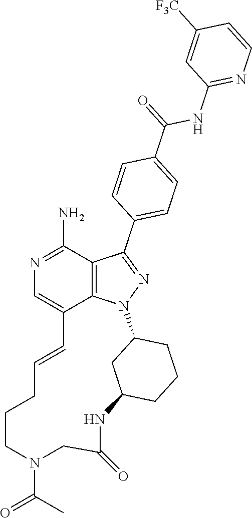 [SMILES: CC(=O)N1CCC/C=C/c2cnc(N)c3c(-c4ccc(C(=O)Nc5cc(C(F)(F)F)ccn5)cc4)nn(c23)[C@@H]2CCC[C@H](C2)NC(=O)C1] | 8.60 | 660.27842 | 661.5 | 10.6 mg | 66.1 |
| 168 | D | L15 | 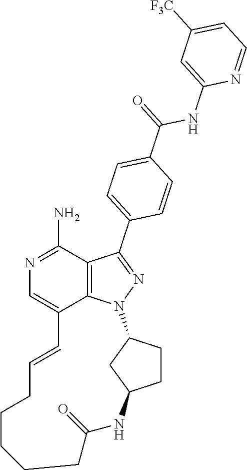 [SMILES: Nc1ncc2c3c1c(-c1ccc(C(=O)Nc4cc(C(F)(F)F)ccn4)cc1)nn3[C@@H]1CC[C@H](C1)NC(=O)CCCCC/C=C/2] | 8.80 | 603.25696 | 604.4 | 33 mg | 39% |
| 169 | D | L24 | 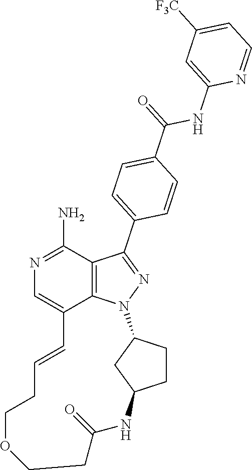 [SMILES: Nc1ncc2c3c1c(-c1ccc(C(=O)Nc4cc(C(F)(F)F)ccn4)cc1)nn3[C@@H]1CC[C@H](C1)NC(=O)CCOCC/C=C/2] | 8.36 | 605.23622 | 606.4 | 49 mg | 51% |
| 170 | D | L23 | 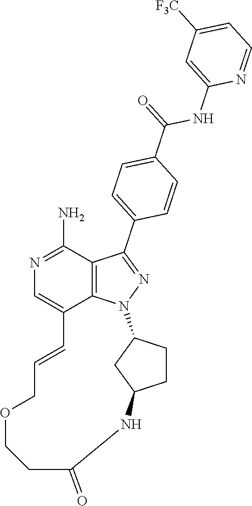 [SMILES: Nc1ncc2c3c1c(-c1ccc(C(=O)Nc4cc(C(F)(F)F)ccn4)cc1)nn3[C@@H]1CC[C@H](C1)NC(=O)CCOC/C=C/2] | 8.31 | 591.22057 | 592.4 | 22 mg | 23% |
| 171 | L | L15 | 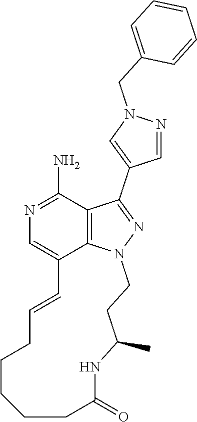 [SMILES: C[C@@H]1CCn2nc(-c3cnn(Cc4ccccc4)c3)c3c(N)ncc(c32)/C=C/CCCCCC(=O)N1] | 7.26 | 483.27466 | 484.4 | 8 mg | 33% |
| 172 | E | L15 | 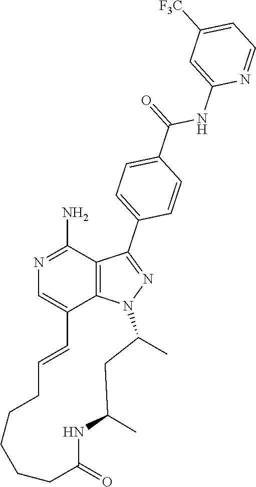 [SMILES: C[C@@H]1C[C@@H](C)n2nc(-c3ccc(C(=O)Nc4cc(C(F)(F)F)ccn4)cc3)c3c(N)ncc(c32)/C=C/CCCCCC(=O)N1] | 9.50 | 605.27261 | 606.4 | 47.7 mg | 45% |
| 173 | L | L15 | 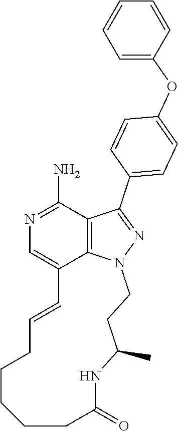 [SMILES: C[C@@H]1CCn2nc(-c3ccc(Oc4ccccc4)cc3)c3c(N)ncc(c32)/C=C/CCCCCC(=O)N1] | 9.24 | 495.26343 | 496.4 | 2 mg | 8% |
| 174 | D | L31 | 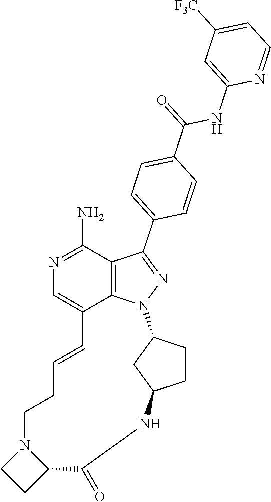 [SMILES: Nc1ncc2c3c1c(-c1ccc(C(=O)Nc4cc(C(F)(F)F)ccn4)cc1)nn3[C@@H]1CC[C@H](C1)NC(=O)[C@@H]1CCN1CC/C=C/2] | 6.84 | 616.25221 | 617.2 | 10 mg | 14% |
| 175 | D | L39 | 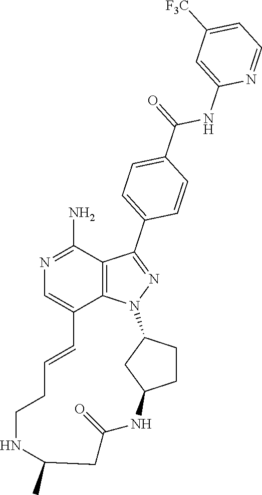 [SMILES: C[C@@H]1CC(=O)N[C@@H]2CC[C@H](C2)n2nc(-c3ccc(C(=O)Nc4cc(C(F)(F)F)ccn4)cc3)c3c(N)ncc(c32)/C=C/CCN1] | 6.77 | 618.26786 | 619.4 | 30 mg | 68% |
| 176 | E | L23 | 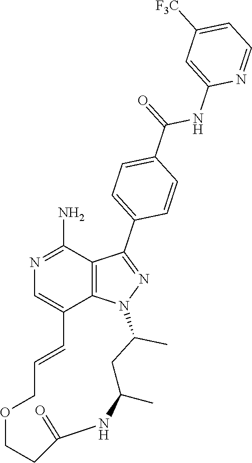 [SMILES: C[C@@H]1C[C@@H](C)n2nc(-c3ccc(C(=O)Nc4cc(C(F)(F)F)ccn4)cc3)c3c(N)ncc(c32)/C=C/COCCC(=O)N1] | 8.75 | 593.23622 | 594.4 | 27.5 mg | 37.6% |
| 177 | J | L24 | 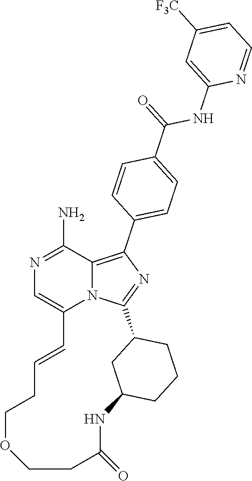 [SMILES: Nc1ncc2n3c(nc(-c4ccc(C(=O)Nc5cc(C(F)(F)F)ccn5)cc4)c13)[C@@H]1CCC[C@H](C1)NC(=O)CCOCC/C=C/2] | 8.27 | 619.25187 | 620.4 | 12 mg | 39% |
| 178 | J | L23 | 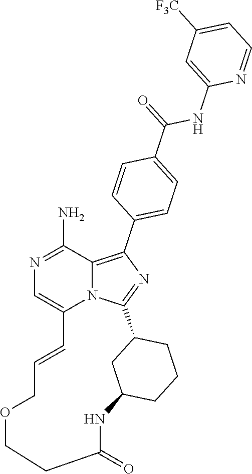 [SMILES: Nc1ncc2n3c(nc(-c4ccc(C(=O)Nc5cc(C(F)(F)F)ccn5)cc4)c13)[C@@H]1CCC[C@H](C1)NC(=O)CCOC/C=C/2] | 8.12 | 605.23622 | 606.4 | 12 mg | 50% |
| 179 | J | L39 | 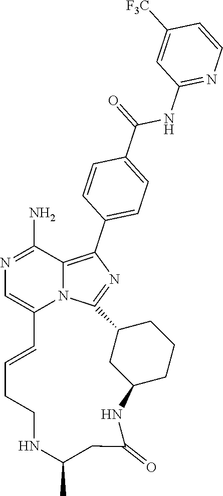 [SMILES: C[C@@H]1CC(=O)N[C@@H]2CCC[C@H](C2)c2nc(-c3ccc(C(=O)Nc4cc(C(F)(F)F)ccn4)cc3)c3c(N)ncc(n23)/C=C/CCN1] | 6.69 | 632.28351 | 633.5 | 8 mg | 42% |
| 180 | B | L39 | 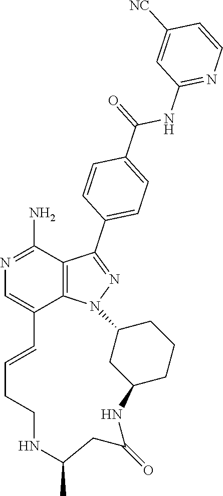 [SMILES: C[C@@H]1CC(=O)N[C@@H]2CCC[C@H](C2)n2nc(-c3ccc(C(=O)Nc4cc(C#N)ccn4)cc3)c3c(N)ncc(c32)/C=C/CCN1] | 5.84 | 589.29137 | 590.5 | 5 mg | 37% |
| 181 | D | L39 | 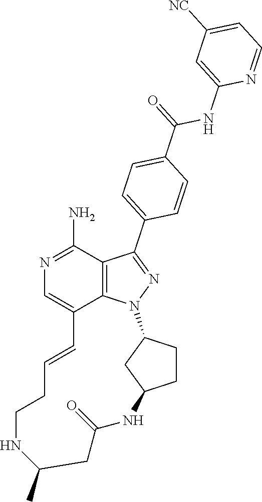 [SMILES: C[C@@H]1CC(=O)N[C@@H]2CC[C@H](C2)n2nc(-c3ccc(C(=O)Nc4cc(C#N)ccn4)cc3)c3c(N)ncc(c32)/C=C/CCN1] | 5.55 | 575.27572 | 576.5 | 4 mg | 16% |
| 182 | D | L15 | 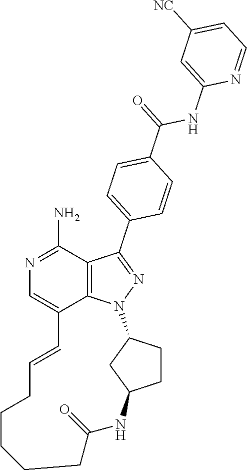 [SMILES: N#Cc1ccnc(NC(=O)c2ccc(-c3nn4c5c(cnc(N)c35)/C=C/CCCCCC(=O)N[C@@H]3CC[C@@H]4C3)cc2)c1] | 7.46 | 560.26482 | 561.4 | 16 mg | 36% |
| 183 | D | L23 | 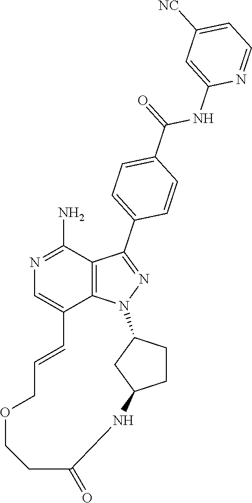 [SMILES: N#Cc1ccnc(NC(=O)c2ccc(-c3nn4c5c(cnc(N)c35)/C=C/COCCC(=O)N[C@@H]3CC[C@@H]4C3)cc2)c1] | 6.91 | 548.22844 | 549.4 | 14 mg | 20% |

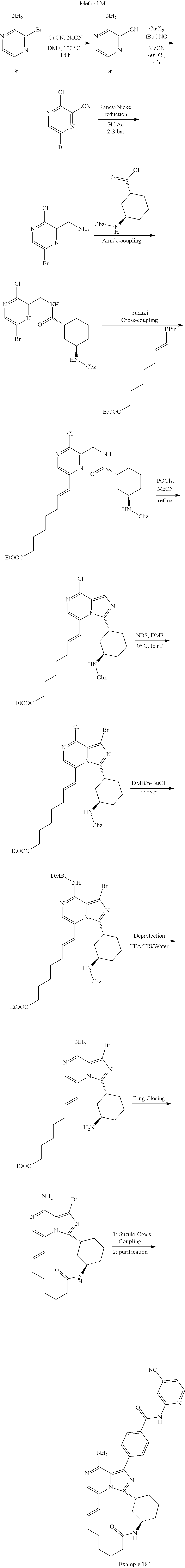

### Example 184

### (a) Ethyl(E)-8-[3-[(1R,3R)-3-(benzyloxycarbonylamino)cyclohexyl]-1-bromo-8-chloro-imidazo[1,5-a]pyrazin-5-yl]oct-7-enoate

This compound was prepared in an analogues manner as described in Method K step a-d, and Method J steps b and c, using Intermediate RP5, Intermediate L15 and (6-bromo-3-chloro-pyrazin-2-yl)methanamine hydrochloride to afford 880 mg of the title compound.

### (b) Ethyl(E)-8-[3-[(1R,3R)-3-(benzyloxycarbonylamino)cyclohexyl]-1-bromo-8-[(2,4-dimethoxyphenyl)methylamino]imidazo[1,5-a]pyrazin-5-yl]oct-7-enoate

To a suspension of ethyl(E)-8-[3-[(1R,3R)-3-(benzyloxycarbonylamino)cyclohexyl]-1-bromo-8-chloro-imidazo[1,5-a]pyrazin-5-yl]oct-7-enoate (924 mg, 1.46 mmol) in 1-butanol (15 mL) was added 2,4-dimethoxybenzylamine (658 μL, 4.38 mmol) and the mixture was stirred at 110° C. for 2 h. The mixture was concentrated under reduced pressure, and the residue was dissolved in ethyl acetate/water 3/1 v/v % (100 mL). The organic layers was separated and washed with 5% NaHCO₃-solution (25 mL) and brine (10 mL), dried over sodium sulfate, filtered and concentrated under reduced pressure. The crude product was purified by column chromatography using SiO₂ and heptane/ethyl acetate=9/1 to 1/4 v/v %. All fractions containing the title compound were collected and concentrated in vacuo to give 1.01 g of the title compound (Yield: 91%).

### (c) Example 184

This compound was prepared in an analogues manner as described in Method J, K and L, starting from ethyl(E)-8-[3-[(1R,3R)-3-(benzyloxycarbonylamino)cyclohexyl]-1-bromo-8-[(2,4-dimethoxyphenyl)methylamino]imidazo[1,5-a]pyrazin-5-yl]oct-7-enoate and using Intermediate BP6 in the last step to afford crude Example 184. Purification was performed using preparative HPLC to afford the title compound (5 mg, 13%). Data: LCMS (B) Rₜ: 7.00 min; m/z575.5 [M+H]⁺.

The following Examples were synthesized following methods as depicted in the table.

| | | | | | | Found | | |
|---|---|---|---|---|---|---|---|---|
| | | Inter- | | Rₜ (min) | | Mass | | |
| Example | Method | mediate | CHEMISTRY | (LCMS B) | Calc Mass | [M + H]⁺ | Amount | Yield |
| 184 | M | L25 | 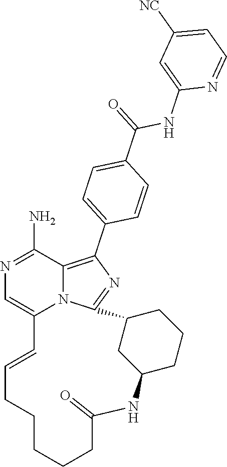 [SMILES: N#Cc1ccnc(NC(=O)c2ccc(-c3nc4n5c(cnc(N)c35)/C=C/CCCCCC(=O)N[C@@H]3CCC[C@@H]4C3)cc2)c1] | 7.00 | 574.28047 | 575.5 | 5 mg | 13% |
| 185 | D | L24 | 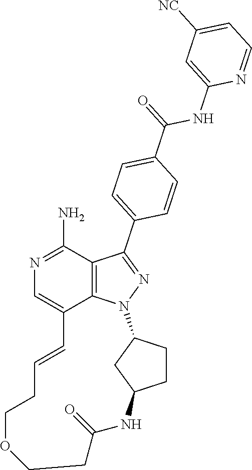 [SMILES: N#Cc1ccnc(NC(=O)c2ccc(-c3nn4c5c(cnc(N)c35)/C=C/CCOCCC(=O)N[C@@H]3CC[C@@H]4C3)cc2)c1] | 6.72 | 562.24409 | 563.4 | 49 mg | 51% |
| 186 | D | L31 | 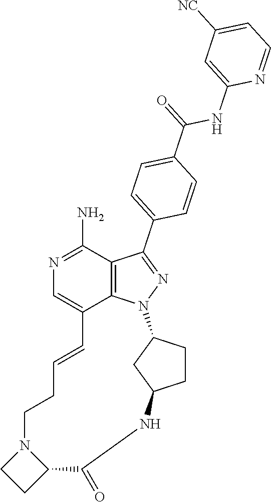 [SMILES: N#Cc1ccnc(NC(=O)c2ccc(-c3nn4c5c(cnc(N)c35)/C=C/CCN3CC[C@H]3C(=O)N[C@@H]3CC[C@@H]4C3)cc2)c1] | 5.26 | 573.26007 | 574.4 | 10 mg | 12% |
| 187 | M | L15 | 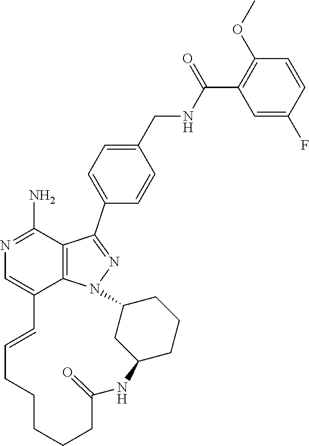 [SMILES: COc1ccc(F)cc1C(=O)NCc1ccc(-c2nn3c4c(cnc(N)c24)/C=C/CCCCCC(=O)N[C@@H]2CCC[C@@H]3C2)cc1] | 8.16 | 610.30677 | 611.4 | 28 mg | 55% |
| 188 | B | L23 | 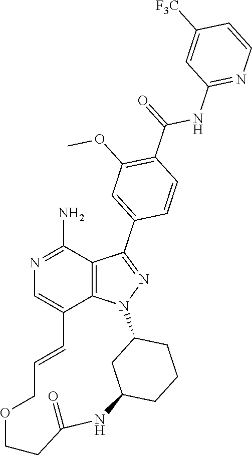 [SMILES: COc1cc(-c2nn3c4c(cnc(N)c24)/C=C/COCCC(=O)N[C@@H]2CCC[C@@H]3C2)ccc1C(=O)Nc1cc(C(F)(F)F)ccn1] | 8.99 | 635.24679 | 636.4 | 36.4 mg | 42.9% |
| 189 | B | L23 | 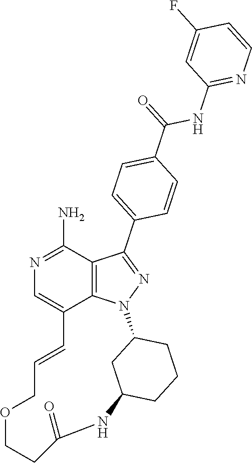 [SMILES: Nc1ncc2c3c1c(-c1ccc(C(=O)Nc4cc(F)ccn4)cc1)nn3[C@@H]1CCC[C@H](C1)NC(=O)CCOC/C=C/2] | 7.08 | 555.23942 | 556.4 | 16 mg | 18.5% |
| 190 | B | L39 | 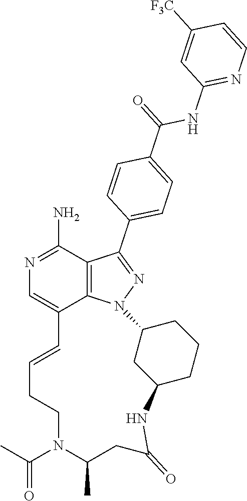 [SMILES: CC(=O)N1CC/C=C/c2cnc(N)c3c(-c4ccc(C(=O)Nc5cc(C(F)(F)F)ccn5)cc4)nn(c23)[C@@H]2CCC[C@H](C2)NC(=O)C[C@H]1C] | 9.06 | 674.29407 | 675.4 | 15 mg | 74% |
| 191 | B | L39 | 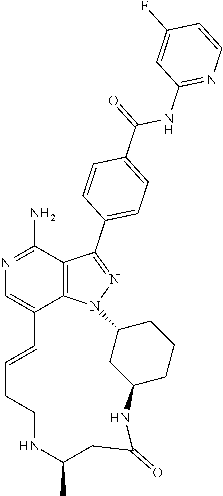 [SMILES: C[C@@H]1CC(=O)N[C@@H]2CCC[C@H](C2)n2nc(-c3ccc(C(=O)Nc4cc(F)ccn4)cc3)c3c(N)ncc(c32)/C=C/CCN1] | 5.76 | 582.28670 | 583.5 | 7.3 mg | 34.6% |
| 192 | D | L23 | 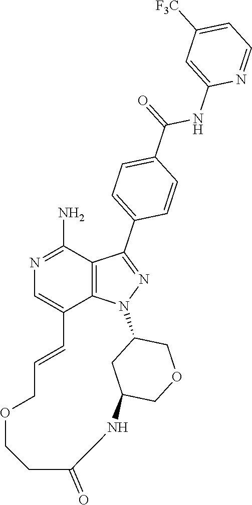 [SMILES: Nc1ncc2c3c1c(-c1ccc(C(=O)Nc4cc(C(F)(F)F)ccn4)cc1)nn3[C@@H]1COC[C@H](C1)NC(=O)CCOC/C=C/2] | 7.69 | 607.21549 | 608.3 | 8.2 mg | 32% |
| 193 | B | L24 | 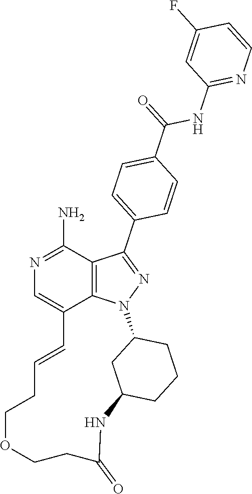 [SMILES: Nc1ncc2c3c1c(-c1ccc(C(=O)Nc4cc(F)ccn4)cc1)nn3[C@@H]1CCC[C@H](C1)NC(=O)CCOCC/C=C/2] | 7.44 | 569.25507 | 570.4 | 27.4 mg | 32.4% |
| 194 | B | L39 | 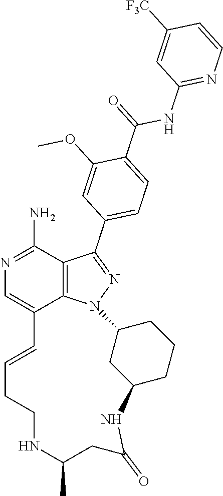 [SMILES: COc1cc(-c2nn3c4c(cnc(N)c24)/C=C/CCN[C@H](C)CC(=O)N[C@@H]2CCC[C@@H]3C2)ccc1C(=O)Nc1cc(C(F)(F)F)ccn1] | 7.46 | 662.29407 | 663.5 | 13.4 mg | 61.1% |
| 195 | B | L24 | 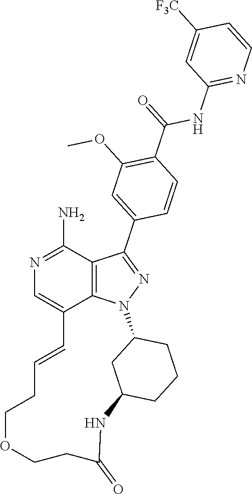 [SMILES: COc1cc(-c2nn3c4c(cnc(N)c24)/C=C/CCOCCC(=O)N[C@@H]2CCC[C@@H]3C2)ccc1C(=O)Nc1cc(C(F)(F)F)ccn1] | 9.19 | 649.26244 | 650.4 | 37.2 mg | 21.5% |
| 196 | B | L15 | 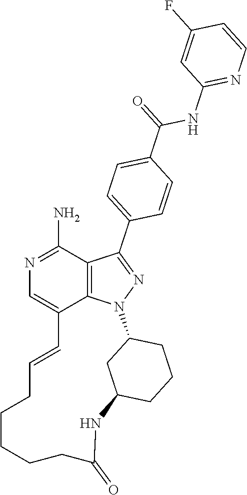 [SMILES: Nc1ncc2c3c1c(-c1ccc(C(=O)Nc4cc(F)ccn4)cc1)nn3[C@@H]1CCC[C@H](C1)NC(=O)CCCCC/C=C/2] | 7.75 | 567.27580 | 568.4 | 30 mg | 28% |
| 197 | B | L15 | 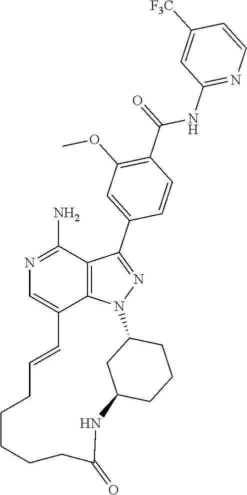 [SMILES: COc1cc(-c2nn3c4c(cnc(N)c24)/C=C/CCCCCC(=O)N[C@@H]2CCC[C@@H]3C2)ccc1C(=O)Nc1cc(C(F)(F)F)ccn1] | 9.80 | 647.28317 | 648.5 | 30.2 mg | 29% |
| 198 | M | L23 | 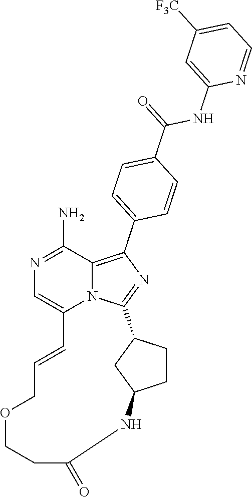 [SMILES: Nc1ncc2n3c(nc(-c4ccc(C(=O)Nc5cc(C(F)(F)F)ccn5)cc4)c13)[C@@H]1CC[C@H](C1)NC(=O)CCOC/C=C/2] | 7.34 | 591.22057 | 592.4 | 1.9 mg | 24% |
| 199 | M | L23 | 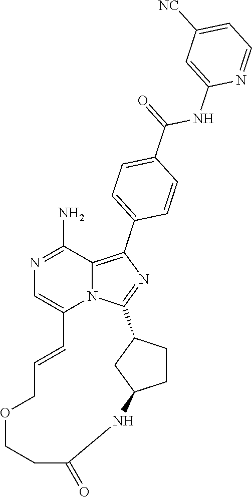 [SMILES: N#Cc1ccnc(NC(=O)c2ccc(-c3nc4n5c(cnc(N)c35)/C=C/COCCC(=O)N[C@@H]3CC[C@@H]4C3)cc2)c1] | 5.77 | 548.22844 | 549.4 | 7.4 mg | 26% |
| 200 | M | L23 | 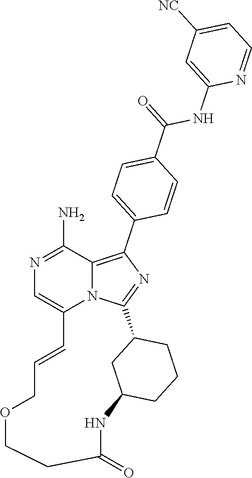 [SMILES: N#Cc1ccnc(NC(=O)c2ccc(-c3nc4n5c(cnc(N)c35)/C=C/COCCC(=O)N[C@@H]3CCC[C@@H]4C3)cc2)c1] | 6.43 | 562.24409 | 563.4 | 4 mg | 15% |
| 201 | M | L24 | 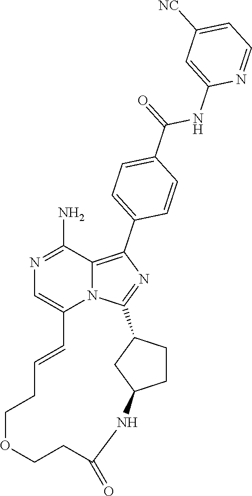 [SMILES: N#Cc1ccnc(NC(=O)c2ccc(-c3nc4n5c(cnc(N)c35)/C=C/CCOCCC(=O)N[C@@H]3CC[C@@H]4C3)cc2)c1] | 6.15 | 562.24409 | 563.4 | 8.2 mg | 31% |
| 202 | M | L24 | 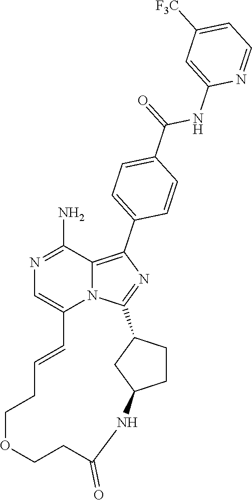 [SMILES: Nc1ncc2n3c(nc(-c4ccc(C(=O)Nc5cc(C(F)(F)F)ccn5)cc4)c13)[C@@H]1CC[C@H](C1)NC(=O)CCOCC/C=C/2] | 7.62 | 605.23622 | 606.4 | 12.9 mg | 28% |
| 203 | M | L15 | 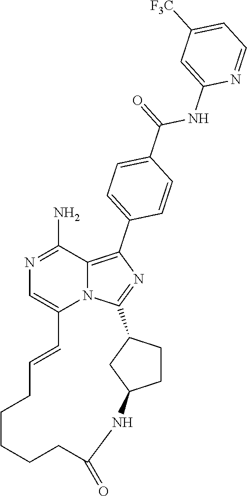 [SMILES: Nc1ncc2n3c(nc(-c4ccc(C(=O)Nc5cc(C(F)(F)F)ccn5)cc4)c13)[C@@H]1CC[C@H](C1)NC(=O)CCCCC/C=C/2] | 3.65 (LCMS A) | 603.25696 | 604.4 | 25 mg | 49% |
| 204 | M | L15 | 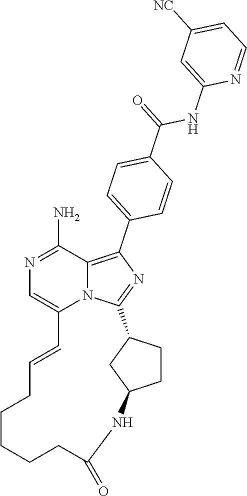 [SMILES: N#Cc1ccnc(NC(=O)c2ccc(-c3nc4n5c(cnc(N)c35)/C=C/CCCCCC(=O)N[C@@H]3CC[C@@H]4C3)cc2)c1] | 6.54 | 560.26482 | 561.4 | 25 mg | 50% |
| 205 | M | L24 | 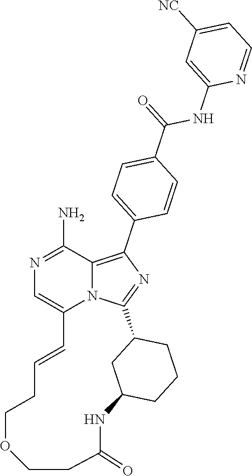 [SMILES: N#Cc1ccnc(NC(=O)c2ccc(-c3nc4n5c(cnc(N)c35)/C=C/CCOCCC(=O)N[C@@H]3CCC[C@@H]4C3)cc2)c1] | 6.54 | 576.25974 | 577.4 | 43 mg | 22% |
| 206 | M | L39 | 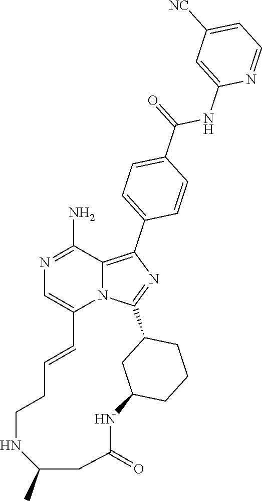 [SMILES: C[C@@H]1CC(=O)N[C@@H]2CCC[C@H](C2)c2nc(-c3ccc(C(=O)Nc4cc(C#N)ccn4)cc3)c3c(N)ncc(n23)/C=C/CCN1] | 4.93 | 589.29137 | 590.5 | 31 mg | 25% |
| 207 | M | L39 | 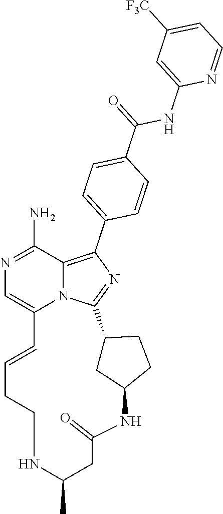 [SMILES: C[C@@H]1CC(=O)N[C@@H]2CC[C@H](C2)c2nc(-c3ccc(C(=O)Nc4cc(C(F)(F)F)ccn4)cc3)c3c(N)ncc(n23)/C=C/CCN1] | 5.87 | 618.26786 | 619.5 | 58 mg | 47% |
| 208 | M | L39 | 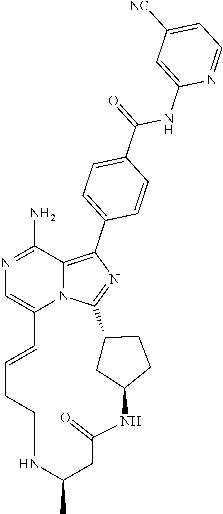 [SMILES: C[C@@H]1CC(=O)N[C@@H]2CC[C@H](C2)c2nc(-c3ccc(C(=O)Nc4cc(C#N)ccn4)cc3)c3c(N)ncc(n23)/C=C/CCN1] | 2.69 (LCMS A) | 575.27572 | 576.5 | 13.8 mg | 8.5% |
| 209 | M | L29 | 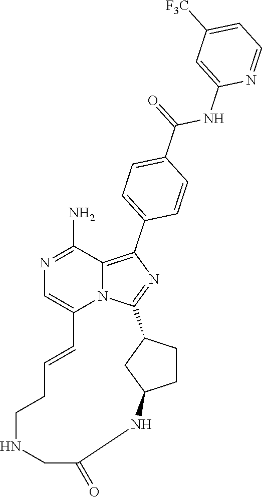 [SMILES: Nc1ncc2n3c(nc(-c4ccc(C(=O)Nc5cc(C(F)(F)F)ccn5)cc4)c13)[C@@H]1CC[C@H](C1)NC(=O)CNCC/C=C/2] | 5.90 | 590.23656 | 591.4 | 45 mg | 76% |
| 210 | M | L24 | 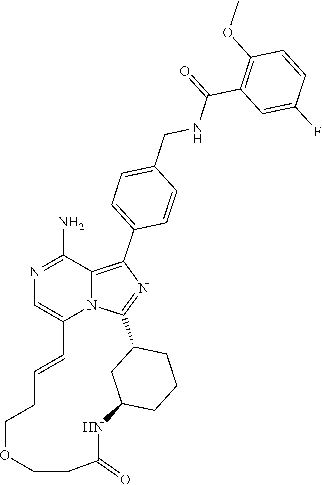 [SMILES: COc1ccc(F)cc1C(=O)NCc1ccc(-c2nc3n4c(cnc(N)c24)/C=C/CCOCCC(=O)N[C@@H]2CCC[C@@H]3C2)cc1] | 7.11 | 612.28603 | 613.5 | 22.4 mg | 77% |
| 211 | M | L23 | 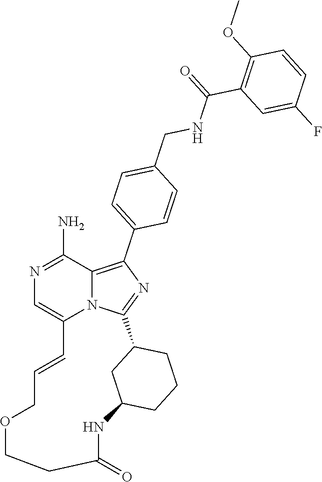 [SMILES: COc1ccc(F)cc1C(=O)NCc1ccc(-c2nc3n4c(cnc(N)c24)/C=C/COCCC(=O)N[C@@H]2CCC[C@@H]3C2)cc1] | 6.86 | 598.27038 | 599.5 | 17.8 mg | 49.8% |
| 212 | M | L39 | 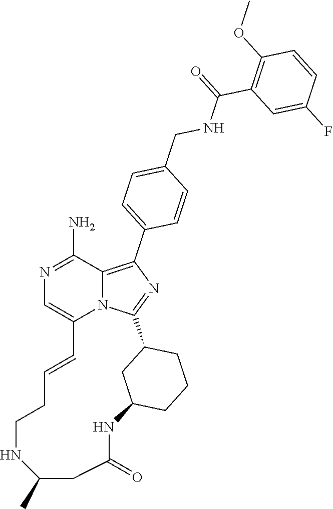 [SMILES: COc1ccc(F)cc1C(=O)NCc1ccc(-c2nc3n4c(cnc(N)c24)/C=C/CCN[C@H](C)CC(=O)N[C@@H]2CCC[C@@H]3C2)cc1] | 5.41 | 625.31767 | 626.5 | 20 mg | 95% |
| 213 | M | L24 | 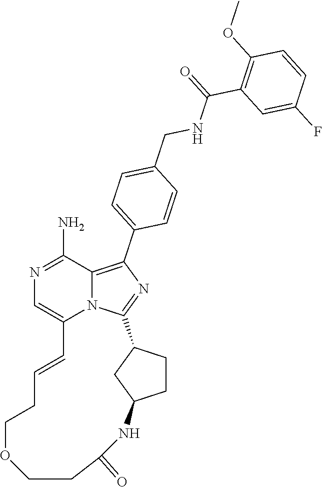 [SMILES: COc1ccc(F)cc1C(=O)NCc1ccc(-c2nc3n4c(cnc(N)c24)/C=C/CCOCCC(=O)N[C@@H]2CC[C@@H]3C2)cc1] | 6.55 | 598.27038 | 599.4 | 22 mg | 71% |
| 214 | M | L39 | 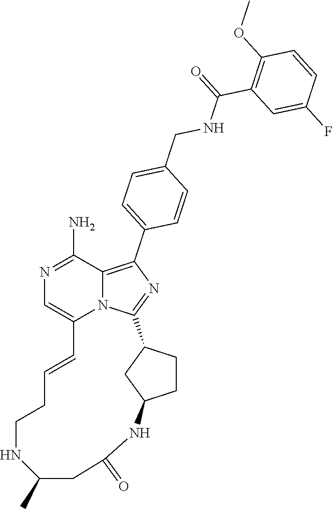 [SMILES: COc1ccc(F)cc1C(=O)NCc1ccc(-c2nc3n4c(cnc(N)c24)/C=C/CCN[C@H](C)CC(=O)N[C@@H]2CC[C@@H]3C2)cc1] | 5.12 | 611.30202 | 612.5 | 9.5 mg | 22% |
| 215 | M | L24 | 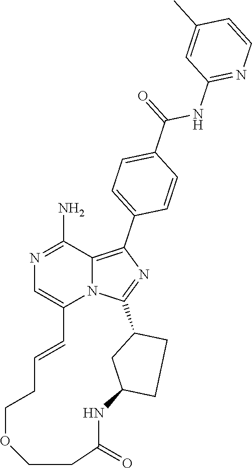 [SMILES: Cc1ccnc(NC(=O)c2ccc(-c3nc4n5c(cnc(N)c35)/C=C/CCOCCC(=O)N[C@@H]3CC[C@@H]4C3)cc2)c1] | 4.70 | 551.26449 | 552.4 | 10.3 mg | 84% |
| 216 | M | L24 | 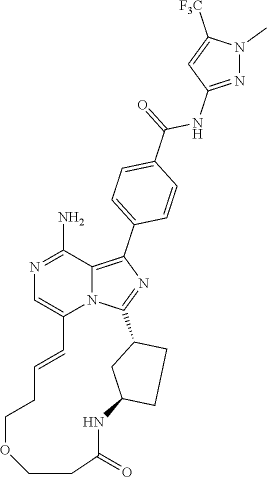 [SMILES: Cn1nc(NC(=O)c2ccc(-c3nc4n5c(cnc(N)c35)/C=C/CCOCCC(=O)N[C@@H]3CC[C@@H]4C3)cc2)cc1C(F)(F)F] | 6.91 | 608.24712 | 609.4 | 7.5 mg | 83.2% |
| 217 | M | L24 | 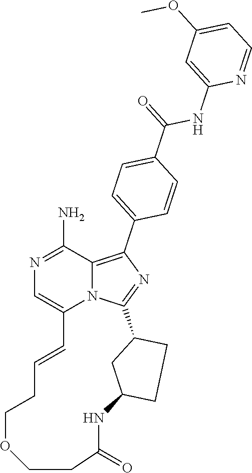 [SMILES: COc1ccnc(NC(=O)c2ccc(-c3nc4n5c(cnc(N)c35)/C=C/CCOCCC(=O)N[C@@H]3CC[C@@H]4C3)cc2)c1] | 4.25 | 567.25940 | 568.4 | 6.1 mg | 99% |
| 218 | M | L39 | 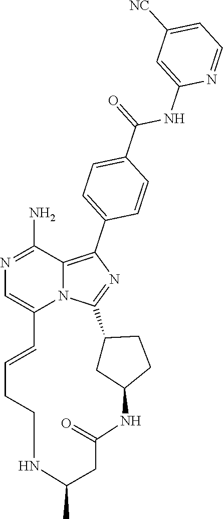 [SMILES: C[C@@H]1CC(=O)N[C@@H]2CC[C@H](C2)c2nc(-c3ccc(C(=O)Nc4cc(C#N)ccn4)cc3)c3c(N)ncc(n23)/C=C/CCN1] | 4.45 | 575.27572 | 576.5 | 32 mg | 26.6% |
| 219 | M | L29 | 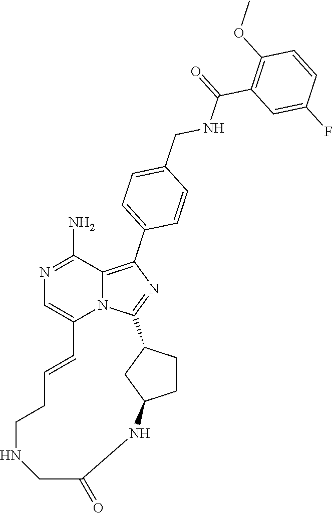 [SMILES: COc1ccc(F)cc1C(=O)NCc1ccc(-c2nc3n4c(cnc(N)c24)/C=C/CCNCC(=O)N[C@@H]2CC[C@@H]3C2)cc1] | 4.84 | 583.27072 | 584.4 | 12 mg | 5% |
| 220 | M | L29 | 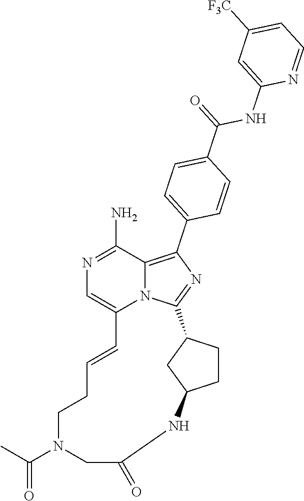 [SMILES: CC(=O)N1CC/C=C/c2cnc(N)c3c(-c4ccc(C(=O)Nc5cc(C(F)(F)F)ccn5)cc4)nc(n23)[C@@H]2CC[C@H](C2)NC(=O)C1] | 7.44 | 632.24712 | 633.4 | 8.4 mg | 53% |
| 221 | M | L29 | 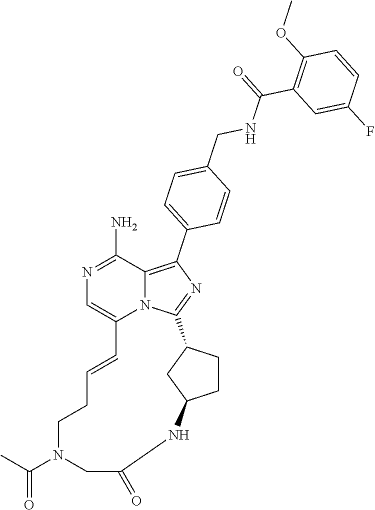 [SMILES: COc1ccc(F)cc1C(=O)NCc1ccc(-c2nc3n4c(cnc(N)c24)/C=C/CCN(C(C)=O)CC(=O)N[C@@H]2CC[C@@H]3C2)cc1] | 6.45 | 625.28128 | 626.4 | 11.5 mg | 71% |
| 222 | M | L24 | 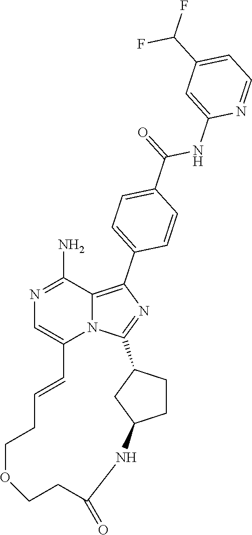 [SMILES: Nc1ncc2n3c(nc(-c4ccc(C(=O)Nc5cc(C(F)F)ccn5)cc4)c13)[C@@H]1CC[C@H](C1)NC(=O)CCOCC/C=C/2] | 6.71 | 587.24564 | 588.4 | 8 mg | 49% |
| 223 | M | L24 | 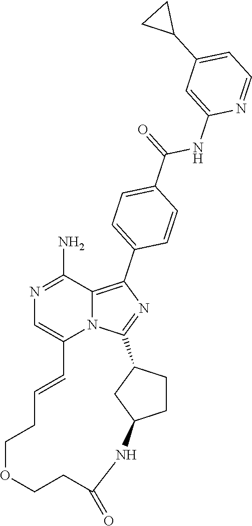 [SMILES: Nc1ncc2n3c(nc(-c4ccc(C(=O)Nc5cc(C6CC6)ccn5)cc4)c13)[C@@H]1CC[C@H](C1)NC(=O)CCOCC/C=C/2] | 5.37 | 577.28014 | 578.5 | 8.4 mg | 48% |
| 224 | M | L24 | 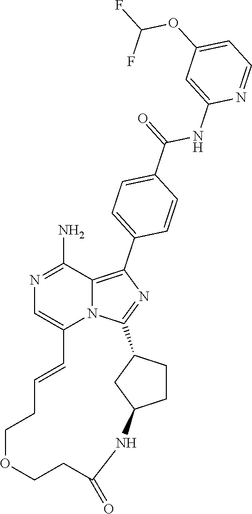 [SMILES: Nc1ncc2n3c(nc(-c4ccc(C(=O)Nc5cc(OC(F)F)ccn5)cc4)c13)[C@@H]1CC[C@H](C1)NC(=O)CCOCC/C=C/2] | 6.66 | 603.24056 | 604.4 | 9 mg | 62% |
| 225 | A,E | L15 | 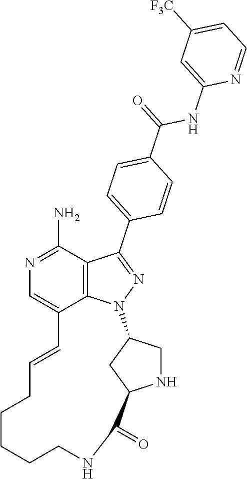 [SMILES: Nc1ncc2c3c1c(-c1ccc(C(=O)Nc4cc(C(F)(F)F)ccn4)cc1)nn3[C@@H]1CN[C@H](C1)C(=O)NCCCCC/C=C/2] | 5.87 | 604.25221 | 605.4 | 10 mg | 9% |
| 226 | A,E | L15 | 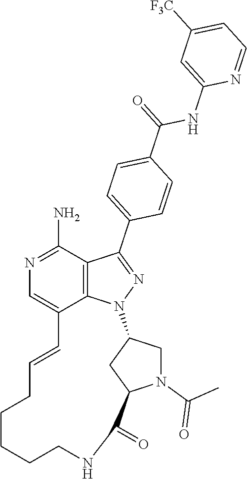 [SMILES: CC(=O)N1C[C@@H]2C[C@@H]1C(=O)NCCCCC/C=C/c1cnc(N)c3c(-c4ccc(C(=O)Nc5cc(C(F)(F)F)ccn5)cc4)nn2c13] | 7.87 | 646.26277 | 647.4 | 5 mg | 29% |

### Example A

### Biochemical kinase assay wt-BTK

To determine the inhibitory activity of compounds on wt-BTK enzyme activity, the IMAP® assay (Molecular Devices) was used. Compounds were serially diluted in dimethylsulfoxide (DMSO) and subsequently in 4% DMSO in IMAP reaction buffer, which consists of 10 mM Tris-HCl, pH 15 7.5, 10 mM MgCl₂, 0.01% Tween-20, 0.1% NaN₃ and 1 mM freshly prepared dithiotreitol (DTT). Compound solution was mixed with an equal volume of full-length wt-BTK enzyme (Carna Biosciences, cat. no. 08-180) in IMAP reaction buffer. After pre-incubation of 1 hour in the dark at room temperature, fluorescein-labeled MBP-derived substrate peptide (Molecular Devices, cat. no. RP 7123) was added, followed by ATP to start the reaction. Final enzyme concentration was 1.2 nM, final substrate concentration 50 nM, and final ATP concentration was 4 μM. The reaction was allowed to proceed for 2 hours at room temperature in the dark. The reaction was stopped by quenching with IMAP progressive binding solution according to the protocol of the manufacturer (Molecular Devices). Fluorescein polarization was measured on an Envision multimode reader (Perkin Elmer, Waltham, MA, U.S.A.). IC₅₀ were calculated using XLfit™5 software (ID Business Solutions, Ltd., Surrey, U.K.).

Results of the wt-BTK binding affinity are shown in Table 1 below.
IC₅₀ wt-BTK: A means IC₅₀<5 nM
B means IC₅₀ between 5 and 50 nM
C means IC₅₀>50 nM and <500 nM

### Example B

### Biochemical kinase assay BTK C481 S

To determine the inhibitory activity of compounds on BTK C481S enzyme activity, the IMAP® assay (Molecular Devices) was used. Compounds were serially diluted in dimethylsulfoxide (DMSO) and subsequently in 4% DMSO in IMAP reaction buffer, which consists of 10 mM Tris-HCl, pH 15 7.5, 10 mM MgCl₂, 0.01% Tween-20, 0.1% NaN₃ and 1 mM freshly prepared dithiotreitol (DTT). Compound solution was mixed with an equal volume of full-length BTK C481S enzyme (Carna Biosciences, cat. no. 08-547) in IMAP reaction buffer. After pre-incubation of 1 hour in the dark at room temperature, fluorescein-labeled MBP-derived substrate peptide (Molecular Devices, cat. no. RP 7123) was added, followed by ATP to start the reaction. Final enzyme concentration was 1.2 nM final substrate concentration 50 nM, and final ATP concentration was 7 μM. The reaction was allowed to proceed for 2 hours at room temperature in the dark. The reaction was stopped by quenching with IMAP progressive binding solution according to the protocol of the manufacturer (Molecular Devices). Fluorescein polarization was measured on an Envision multimode reader (Perkin Elmer, Waltham, MA, U.S.A.). IC₅₀ were calculated using XLfit™5 software (ID Business Solutions, Ltd., Surrey, U.K.).

Results of the BTK C481 S binding affinity are shown in Table 1 below.
IC₅₀ BTK C481 S: A means IC₅₀<5 nM
B means IC₅₀ between 5 and 50 nM
C means IC₅₀>50 nM and <500 nM
Example C
   Cell proliferation assay

wt-REC-1 mantle cell lymphoma cells were purchased from American Type Culture Collection via Synthego Corporation (cat. no. CRL-3004, ATCC). Frozen stocks were thawed and cells were diluted in RPMI-1640 cell culture medium (cat. no. 61870036, Life Technologies), supplemented with 10% (v/v) fetal calf serum and 1% penicillin/streptavidin. 3200 cells per well (in 45 μl) were seeded in a white 384-well culture plate (cat. no. 781080, Greiner Bio-One) and allowed to rest for 24 hours at 37° C., 95% humidity, and 5% CO₂. 5 μl compound solution was added to the cells and incubation was continued for 72 hours (3 days), followed by addition of 24 μl ATPlite 1Step™ (PerkinElmer, Groningen, The Netherlands) solution was added to each well. Luminescence was recorded on an Envision multimode reader. The cell signal at the start of incubation was recorded separately in order to distinguish between cell population growth and cell death. In addition, maximum growth was determined by incubation of a duplicate without compound in the presence of 0.3% DMSO. Percentage growth was used as the main y-axis signal. IC₅₀ₛ were fitted by non-linear regression using IDBS XLfit™5 using a 4-parameter logistic curve, yielding a maximum signal, minimum signal, hill-parameter and IC₅₀.

Results of the wt-REC-1 proliferation are shown in Table 1 below.
IC₅₀ wt-REC-1: A means IC₅₀<100 nM
B means IC₅₀ between 100 nM and 1 μM
C means IC₅₀>1 μM and <10 μM

### Example D

Cell proliferation assay
Generation of BTK T474I knock-in cell lines

wt-REC-1 cell lines expressing mutant BTK were created at Synthego Corporation. Cell lines expressing BTK T474I was generated via CRISPR/Cas9. Monoclonal REC-1 T474I cell lines were obtained by single cell cloning. The mutation status of all cell lines was confirmed via sequencing. Frozen stocks were thawed and cells were diluted in RPMI-1640 cell culture medium (cat. no. 61870036, Life Technologies), supplemented with 10% (v/v) fetal calf serum and 1% penicillin/streptavidin. 3200 cells per well (in 45 μl) were seeded in a white 384-well culture plate (cat. no. 781080, Greiner Bio-One) and allowed to rest for 24 hours at 37° C., 95% humidity, and 5% CO₂. 5 μl compound solution was added to the cells and incubation was continued for 72 hours (3 days), followed by addition of 24 μl ATPlite 1Step™ (PerkinElmer, Groningen, The Netherlands) solution was added to each well. Luminescence was recorded on an Envision multimode reader. The cell signal at the start of incubation was recorded separately in order to distinguish between cell population growth and cell death. In addition, maximum growth was determined by incubation of a duplicate without compound in the presence of 0.3% DMSO. Percentage growth was used as the main y-axis signal. IC₅₀ₛ were fitted by non-linear regression using IDBS XLfit™ m5 using a 4-parameter logistic curve, yielding a maximum signal, minimum signal, hill-parameter and IC₅₀.
Results of the REC-1 T474I proliferation are shown in Table 1 below.
IC₅₀ REC-1 T474A means IC₅₀<100 nM
B means IC₅₀ between 100 nM and 500 nM
C means IC₅₀>500 nM and <10 μM

TABLE 1
IC₅₀ values for in vitro biochemical and cellular assays.

| | IC₅₀ wt- | IC₅₀ BTK | IC₅₀ wt- | IC₅₀ REC-1 |
|---|---|---|---|---|
| Example | BTK | C481S | REC-1 | T474I |
| 1 | A | A | A | A |
| 2 | A | A | A | A |
| 3 | A | A | B | C |
| 4 | B | B | B | C |
| 5 | A | A | A | B |
| 6 | A | A | A | B |
| 7 | A | A | A | C |
| 8 | A | A | B | C |
| 9 | A | A | B | C |
| 10 | A | A | A | C |
| 11 | A | A | A | B |
| 12 | A | A | A | C |
| 13 | A | A | B | C |
| 14 | A | A | B | C |
| 15 | A | A | B | |
| 16 | A | A | A | A |
| 17 | A | A | A | A |
| 18 | A | A | A | A |
| 19 | B | B | B | C |
| 20 | A | A | B | C |
| 21 | A | A | A | B |
| 22 | A | A | B | C |
| 23 | A | A | A | B |
| 24 | A | A | A | A |
| 25 | A | A | A | A |
| 26 | A | A | A | A |
| 27 | A | A | A | A |
| 28 | A | A | A | B |
| 29 | A | A | A | B |
| 30 | A | A | A | B |
| 31 | A | A | A | A |
| 32 | A | A | A | B |
| 33 | A | A | A | A |
| 34 | A | A | A | A |
| 35 | A | A | A | B |
| 36 | A | A | A | B |
| 37 | A | A | A | A |
| 38 | A | A | A | A |
| 39 | A | A | B | C |
| 40 | A | A | A | A |
| 41 | A | A | A | A |
| 42 | A | A | A | B |
| 43 | A | A | B | C |
| 44 | A | A | A | A |
| 45 | A | A | A | A |
| 46 | A | A | A | A |
| 47 | A | A | B | B |
| 48 | A | A | A | A |
| 49 | A | A | A | B |
| 50 | A | A | B | C |
| 51 | A | A | B | B |
| 52 | A | A | B | C |
| 53 | A | A | A | B |
| 54 | A | A | A | B |
| 55 | A | A | B | C |
| 56 | A | A | A | C |
| 57 | A | A | A | C |
| 58 | B | B | C | C |
| 59 | A | A | A | C |
| 60 | A | A | A | C |
| 61 | A | A | A | |
| 62 | A | A | B | C |
| 63 | A | A | B | C |
| 64 | B | A | B | C |
| 65 | A | A | C | C |
| 66 | A | A | C | C |
| 67 | A | A | C | C |
| 68 | A | A | A | C |
| 69 | A | A | B | B |
| 70 | A | A | A | C |
| 71 | A | A | A | B |
| 72 | A | A | A | C |
| 73 | A | A | A | B |
| 74 | A | A | B | C |
| 75 | A | A | B | C |
| 76 | A | A | B | C |
| 77 | B | B | B | C |
| 78 | A | A | B | C |
| 79 | A | A | A | B |
| 80 | A | A | A | A |
| 81 | A | A | A | B |
| 82 | A | A | B | C |
| 83 | A | A | A | C |
| 84a | B | B | B | C |
| 84b | B | A | B | |
| 85a | B | C | B | C |
| 85b | A | A | A | B |
| 86 | B | C | B | C |
| 87a | C | C | C | C |
| 87b | A | A | A | B |
| 88a | B | B | B | C |
| 88b | A | A | A | C |
| 89a | B | B | B | C |
| 89b | A | A | A | |
| 90 | B | B | B | C |
| 91 | C | C | C | C |
| 92 | B | B | B | C |
| 93 | B | A | B | C |
| 94a | B | B | B | C |
| 94b | A | A | B | C |
| 95 | B | B | C | C |
| 96b | B | B | C | C |
| 96a | C | C | C | C |
| 97a | B | B | C | C |
| 97b | B | A | B | C |
| 98a | C | C | B | C |
| 98b | A | A | B | |
| 99a | C | C | C | C |
| 99b | B | B | B | C |
| 100a | C | C | C | C |
| 100b | A | A | B | C |
| 101a | B | B | B | C |
| 101b | A | A | B | C |
| 102a | A | A | A | A |
| 102b | B | A | B | C |
| 103a | A | A | B | B |
| 103b | A | A | B | B |
| 104a | C | C | C | C |
| 104b | C | B | B | C |
| 105 | B | B | C | C |
| 106 | B | B | C | C |
| 107 | B | B | C | C |
| 108 | A | A | B | C |
| 109a | B | B | B | C |
| 109b | C | C | C | C |
| 110 | B | B | B | C |
| 111 | B | B | C | C |
| 112 | A | A | B | B |
| 113 | B | B | C | C |
| 114 | A | A | B | C |
| 115 | A | A | B | B |
| 116 | C | C | C | C |
| 117 | B | A | B | C |
| 118 | A | A | A | C |
| 119 | A | A | A | C |
| 120 | B | B | B | C |
| 121 | B | A | B | C |
| 122 | B | B | B | C |
| 123 | A | A | B | C |
| 124 | C | C | B | C |
| 125 | B | B | B | C |
| 126 | A | A | A | B |
| 127 | A | A | B | C |
| 128 | A | A | B | B |
| 129 | A | A | B | C |
| 130 | A | A | A | |
| 131 | A | A | A | A |
| 132 | A | A | B | C |
| 133a | B | B | B | C |
| 133b | A | A | A | |
| 134 | B | B | C | |
| 135 | B | B | C | |
| 136 | B | B | C | |
| 137 | A | A | A | A |
| 138 | B | B | A | C |
| 139 | A | A | B | B |
| 140 | A | A | C | C |
| 141 | A | A | A | A |
| 142 | A | A | B | C |
| 143 | A | A | B | C |
| 144 | B | B | C | C |
| 145 | A | A | C | C |
| 146 | A | A | A | B |
| 147 | A | A | A | A |
| 148 | A | A | B | C |
| 149 | A | A | B | A |
| 150 | A | A | B | B |
| 151 | A | A | B | B |
| 152 | A | A | B | A |
| 153 | A | A | B | C |
| 154 | A | A | C | C |
| 155 | A | A | A | A |
| 156 | A | A | A | A |
| 157 | A | A | A | A |
| 158 | A | A | B | C |
| 159 | A | A | B | C |
| 160 | A | A | A | B |
| 161 | A | A | A | A |
| 162 | A | A | A | A |
| 163 | A | A | A | A |
| 164 | A | A | A | A |
| 165 | A | A | A | A |
| 166 | A | A | A | A |
| 167 | A | A | A | B |
| 168 | A | A | A | A |
| 169 | A | A | A | A |
| 170 | A | A | A | A |
| 171 | C | C | C | C |
| 172 | A | A | A | A |
| 173 | A | A | A | C |
| 174 | A | A | A | A |
| 175 | A | A | A | B |
| 176 | A | A | A | A |
| 177 | A | A | A | B |
| 178 | A | A | A | B |
| 179 | A | A | A | B |
| 180 | A | A | A | B |
| 181 | A | A | A | C |
| 182 | A | A | A | A |
| 183 | A | A | A | A |
| 184 | A | A | A | A |
| 185 | A | A | A | A |
| 186 | A | A | A | A |
| 187 | A | A | A | B |
| 188 | A | A | A | A |
| 189 | A | A | C | C |
| 190 | A | A | C | C |
| 191 | A | A | A | A |
| 192 | A | A | A | A |
| 193 | A | A | A | A |
| 194 | A | A | A | C |
| 195 | A | A | A | A |
| 196 | A | A | A | A |
| 197 | A | A | A | B |
| 198 | A | A | A | |
| 199 | A | A | A | C |
| 200 | A | A | A | C |
| 201 | A | A | A | A |
| 202 | A | A | A | A |
| 203 | A | A | A | A |
| 204 | A | A | A | A |
| 205 | A | A | A | A |
| 206 | A | A | A | C |
| 207 | A | A | A | C |
| 208 | A | A | C | C |
| 209 | A | A | A | C |
| 210 | A | A | A | A |
| 211 | A | A | A | C |
| 212 | A | A | A | C |
| 213 | A | A | A | A |
| 214 | A | A | B | C |
| 215 | A | A | A | A |
| 216 | A | A | A | A |
| 217 | A | A | A | A |
| 218 | A | A | A | C |
| 219 | A | A | B | C |
| 220 | A | A | C | C |
| 221 | A | A | C | C |
| 222 | A | A | A | B |
| 223 | A | A | A | B |
| 224 | A | A | A | A |
| 225 | A | A | A | C |
| 226 | A | A | C | C |

### Example E

Binding kinetics measurements on wt-BTK, BTK C481 S, BTK T316A, BTK T474I and BTK T474S (Surface Plasmon Resonance)

Streptavidin-coated chips (Cat. No. BR100531), disposables and maintenance kits for Biacore were purchased from Cytiva (Eindhoven, The Netherlands). Biotinylated wt-BTK enzym (Carna Biosciences, cat. no. 08-480-20N), BTK C481S (Carna Biosciences, cat. no. 08-417-20N), BTK T316A (Carna Biosciences, cat. no. 08-418-20N), BTK T474I (Carna Biosciences, cat. no. 08-419-20N) or BTK T474S (Carna Biosciences, cat. no. 08-420-20N) were immobilized on a streptavidin-coated chip to a level of about 8000 resonance units (RU) using Biacore buffer (50 mM Tris pH 7.5, 0.05% (v/v) Tween-20, 150 mM NaCl and 5 mM MgCl₂)+1 mM TCEP. Remaining streptavidin was blocked with biocytin. Immobilization was performed at 4° C. Subsequent assay steps were conducted at 22° C. After changing buffer to Biacore buffer with 1% (v/v) dimethylsulfoxide (DMSO), a pre-run was performed for a period of at least 30 min at a flow rate of 30 μl/min to obtain a stable surface. The kinetic constants of the compounds were determined with single cycle kinetics with five consecutive injections with an increasing compound concentration with ranges of 3.16-316 nM. Experiments were performed with an association time of 100 s per concentration and a dissociation time of 1200 s, except for compounds with a long target residence time, such as irreversible inhibitors, where dissociation time was increased. To circumvent problems of mass transport limitation, a flow rate of 30 μl/min was used. A blank run with the same conditions was performed before the compound was injected. The SPR sensorgrams were analyzed with Biacore Evaluation Software by using a method of double referencing. First the reference channel was subtracted from the channel containing immobilized protein. Subsequently, the reference curve obtained with buffer injections was subtracted. The resulting curve was fitted with a 1:1 binding model. Compounds that bound according to an induced fit model were fitted with a two-state reaction model. The kinetic constants (kₐ, kₐ, K_{D}) of duplicates were geometrically averaged. Target residence time (T) for the 1:1 binding model was calculated from the dissociation constant k_{d} with the formula T=1/k_{d}. Target residence for an induced fit model was calculated as described (Tummino and Copeland, 2008).

Compounds of examples 1, 2, 6, 7, 8, 9,10, 11, 17, 18, 20, 21, 24, 25, 26, 27, 28, 33, 34, 39, 40, 44, 45, 46, 47, 48, 49, 50, 51, 52, 53, 54, 56, 57, 59, 61, 68, 71, 76, 79, 83, 85b, 87b, 88b, 98b, 100b, 114, 115, 130, 131, 132, 133b, 139, 141, 146, 147, 150, 151, 152, 153, 155, 157, 162-165, 168-170, 172, 174-176, 179-187, 189, 192, 193, 195, 198-201, 203-205, 207, 210, 211, 213, 215-217 and 222-226 showed a K_{D} (wt-BTK) value of <5 nM.

Compounds of examples 1, 6, 7, 8, 9,10,17, 20, 21, 24, 25, 26, 27, 28, 33, 34, 39, 44, 45, 46, 47, 48, 49, 50, 51, 53, 54, 56, 57, 59, 61, 68, 71, 76, 79, 83, 85b, 87b, 88b, 89b, 98b, 100b, 114, 115, 130, 131, 132, 133b, 139, 141, 146, 147, 150, 151, 152, 153, 155, 157, 162-165, 168-170, 172, 174, 175, 179, 183, 184, 198-202, 204 and 207 showed a K_{D} (BTK C481S) value of <5 nM.

Compounds of examples 1, 2, 6, 7, 8, 9,10, 11, 17, 18, 20, 21, 24, 25, 26, 27, 28, 33, 34, 39, 40, 44, 45, 46, 47, 48, 49, 50, 51, 53, 54, 56, 57, 59, 61, 68, 71, 76, 79, 83, 87b, 88b, 89b, 98b, 10b, 115, 130, 131, 132, 133b, 139,141,146,147,150,152,153,155,157,162-165, 168-170, 172 and 174-176 showed a K_{D} (BTK T31 6A) value of <5 nM.

Compounds of examples 8, 9, 50, 51, 52, 53, 56, 59, 76, 85b, 87b, 88b, 89b, 98b, 100b, 114,115,139,146,156and 211 showed K_{D} (BTK T474)value7>0nMand<5 nM,and compounds of examples 1, 2, 6, 7, 10, 11, 17, 18, 20, 21, 24, 25, 26, 27, 28, 33, 34, 39, 40, 44, 45, 46, 47, 48, 49, 54, 57, 61, 68, 71, 79, 83, 130, 131, 132, 133b, 141, 147, 150, 151, 152, 153, 155, 157,162-165,168-170,172,174-176,179,180,182-189,192,193, 198-205, 207, 1 210, 213,215-217and 222-226showedaKD(BTKT474)value,<2 nM.

Compounds of examples 6, 7, 8, 9, 10, 11, 17, 18, 20, 21, 24, 25, 26, 27, 28, 33, 34, 39, 40, 44, 45, 46, 47, 48, 49, 50, 51, 52, 53, 54, 56, 57, 59, 61, 68, 71, 76, 79, 83, 85b, 87b, 88b, 98b, 10b, 115, 130, 131, 132, 133b, 139, 141, 146, 147, 150, 151, 152, 153, 155, 156, 157, 162-165,168-170,172,174-176,179-181, 83and 87showed a K_{D} (BTK T474S)value of 4<3 nM.

Target residence times for representative examples are given in Table 2a-e below.

TABLE 2a
Binding Kinetics (SPR measurements) Target residence
times (τ (h)) for wt-BTK

| | Tau (h) | | Tau (h) |
|---|---|---|---|
| Example | wt-BTK (Act) | Example | wt-BTK (Act) |
| 1 | 8.77 | 150 | 2.52 |
| 2 | 4.47 | 151 | 7.58 |
| 6 | 6.80 | 152 | 2.36 |
| 7 | 2.57 | 153 | 1.29 |
| 8 | 2.25 | 155 | 9.97 |
| 9 | 1.02 | 157 | 5.15 |
| 10 | 6.77 | 162 | 3.41 |
| 11 | 5.48 | 163 | >24 |
| 17 | 5.24 | 164 | 10.55 |
| 18 | 4.53 | 165 | 3.24 |
| 20 | 2.79 | 168 | 3.66 |
| 21 | 4.34 | 169 | 2.34 |
| 24 | 4.78 | 170 | 2.05 |
| 25 | 9.45 | 172 | 4.83 |
| 26 | 5.89 | 174 | 1.35 |
| 27 | 5.05 | 176 | 3.13 |
| 28 | 4.52 | 179 | 7.36 |
| 33 | 3.46 | 180 | 7.42 |
| 34 | 6.06 | 181 | 2.95 |
| 39 | 4.22 | 182 | 2.96 |
| 40 | 6.49 | 183 | >24 |
| 44 | 2.32 | 184 | 5.55 |
| 45 | 4.42 | 185 | 10.79 |
| 46 | 4.87 | 186 | >24 |
| 47 | 2.66 | 187 | 18.83 |
| 48 | 7.37 | 189 | 4.17 |
| 49 | 3.20 | 192 | 3.61 |
| 51 | 1.82 | 193 | 6.70 |
| 53 | 0.89 | 195 | 3.73 |
| 54 | 3.03 | 198 | 4.28 |
| 56 | 2.24 | 199 | 1.69 |
| 57 | 2.14 | 200 | 2.12 |
| 59 | 1.10 | 201 | 10.14 |
| 61 | 4.95 | 202 | 2.20 |
| 68 | 4.48 | 203 | 5.73 |
| 71 | 7.08 | 204 | 5.64 |
| 76 | 0.97 | 205 | 1.80 |
| 79 | 8.24 | 207 | 2.55 |
| 83 | 2.95 | 210 | 1.22 |
| 85b | 4.08 | 213 | 2.29 |
| 88b | 1.37 | 215 | 9.76 |
| 98b | 4.35 | 216 | 4.79 |
| 115 | 5.09 | 217 | 2.78 |
| 130 | 2.86 | 222 | 5.31 |
| 131 | 3.70 | 223 | 2.52 |
| 132 | 2.75 | 224 | 3.69 |
| 133b | 4.39 | 225 | 3.59 |
| 139 | 1.38 | 226 | 2.76 |
| 141 | >24 | Pirtobrutinib | 0.45 |
| 146 | 3.88 | Vecabrutinib | 0.18 |
| 147 | 5.28 | Fenebrutinib | 0.29 |

TABLE 2b
Binding Kinetics (SPR measurements) Target residence
times (τ (h)) for BTK C481S

| | Tau (h) BTK | | Tau (h) BTK |
|---|---|---|---|
| Example | C418S (Act) | Example | C418S (Act) |
| 1 | 1.24 | 114 | 1.85 |
| 6 | 2.91 | 115 | 6.25 |
| 7 | 4.50 | 130 | 3.83 |
| 8 | 3.30 | 131 | 5.25 |
| 9 | 2.60 | 132 | 3.96 |
| 10 | 6.76 | 133b | 7.08 |
| 17 | 14.35 | 139 | 1.36 |
| 20 | 1.05 | 141 | 4.45 |
| 21 | 7.53 | 146 | 5.76 |
| 24 | 10.68 | 147 | 1.73 |
| 25 | 12.17 | 150 | 1.51 |
| 26 | 6.65 | 151 | 8.25 |
| 27 | 2.04 | 152 | 5.84 |
| 28 | 4.03 | 153 | 1.67 |
| 33 | 9.96 | 155 | 2.21 |
| 34 | 9.06 | 157 | 2.42 |
| 39 | 2.54 | 162 | 6.32 |
| 45 | 3.17 | 163 | 3.51 |
| 46 | 9.36 | 164 | >24 |
| 47 | 4.68 | 165 | 1.61 |
| 48 | 4.27 | 168 | 6.64 |
| 49 | 4.75 | 169 | 6.60 |
| 51 | 1.92 | 170 | 2.76 |
| 53 | 1.57 | 172 | 4.28 |
| 54 | 3.40 | 174 | 3.56 |
| 56 | 2.74 | 175 | 4.68 |
| 57 | 7.92 | 179 | 4.06 |
| 59 | 2.05 | 183 | 4.15 |
| 61 | 7.19 | 184 | 2.43 |
| 68 | 2.39 | 198 | 18.16 |
| 71 | 4.52 | 199 | 1.28 |
| 76 | 1.63 | 200 | 2.23 |
| 79 | 7.11 | 201 | 1.91 |
| 83 | 4.44 | 202 | 3.15 |
| 85b | 7.12 | 207 | 8.44 |
| 88b | 1.44 | Pirtobrutinib | 0.91 |
| 89b | 6.69 | Vecabrutinib | 0.12 |
| 98b | 5.98 | Fenebrutinib | 0.08 |
| 100b | 0.98 | | |

TABLE 2c
Binding Kinetics (SPR measurements) Target residence
times (τ (h)) for BTK T316A

| | Tau (h) BTK | | Tau (h) BTK |
|---|---|---|---|
| Example | T316A (Act) | Example | T316A (Act) |
| 1 | 2.99 | 79 | 3.23 |
| 2 | 16.05 | 83 | 1.36 |
| 6 | 2.28 | 85b | 2.50 |
| 7 | 2.03 | 89b | 3.12 |
| 8 | 1.30 | 98b | 3.58 |
| 10 | 1.76 | 115 | 2.96 |
| 11 | 6.42 | 130 | 2.22 |
| 17 | 3.60 | 131 | 3.44 |
| 18 | 4.74 | 132 | 1.58 |
| 20 | 2.01 | 133b | 1.96 |
| 21 | 3.89 | 141 | 8.86 |
| 24 | 2.66 | 146 | 2.12 |
| 25 | 3.55 | 147 | 10.42 |
| 26 | 3.22 | 150 | 2.48 |
| 27 | 3.37 | 151 | 1.89 |
| 28 | 1.43 | 152 | 6.08 |
| 33 | 3.62 | 155 | 2.91 |
| 34 | 5.41 | 156 | 1.05 |
| 39 | 1.25 | 157 | 5.27 |
| 40 | 2.16 | 162 | 7.64 |
| 44 | 2.47 | 163 | 4.67 |
| 45 | 6.06 | 164 | 3.41 |
| 46 | 3.23 | 165 | 6.98 |
| 47 | 1.91 | 168 | 3.81 |
| 48 | 3.89 | 169 | 1.77 |
| 49 | 2.20 | 170 | 1.58 |
| 54 | 2.24 | 172 | 2.18 |
| 56 | 1.46 | 174 | 4.98 |
| 57 | 2.06 | 175 | 3.51 |
| 59 | 1.04 | 176 | 5.38 |
| 61 | 1.85 | Pirtobrutinib | 0.17 |
| 68 | 3.79 | Vecabrutinib | 0.05 |
| 71 | 4.64 | Fenebrutinib | 0.03 |

TABLE 2d
Binding Kinetics (SPR measurements) Target residence
times (τ (h)) for BTK T474I

| | Tau (h) BTK | | Tau (h) BTK |
|---|---|---|---|
| Example | T474I (Act) | Example | T474I (Act) |
| 1 | 0.92 | 174 | 0.77 |
| 2 | 3.61 | 175 | 1.31 |
| 11 | 1.93 | 176 | 1.94 |
| 17 | 1.12 | 179 | 1.14 |
| 18 | 0.63 | 180 | 1.16 |
| 24 | 1.26 | 182 | 1.07 |
| 25 | 3.02 | 183 | >24 |
| 26 | 5.95 | 184 | 0.85 |
| 27 | 2.55 | 185 | 1.26 |
| 28 | 0.73 | 186 | 0.93 |
| 33 | 1.26 | 187 | 2.13 |
| 34 | 1.70 | 188 | >24 |
| 40 | 0.85 | 189 | 1.56 |
| 44 | 0.87 | 192 | 1.40 |
| 45 | 2.64 | 193 | >24 |
| 46 | 2.58 | 195 | 0.73 |
| 47 | 0.86 | 198 | 1.44 |
| 48 | 2.01 | 199 | 0.54 |
| 71 | 0.52 | 200 | 0.77 |
| 79 | 1.80 | 201 | 1.15 |
| 89b | 0.64 | 202 | 2.16 |
| 98b | 1.14 | 203 | 4.17 |
| 131 | 2.37 | 204 | 0.90 |
| 141 | 6.21 | 205 | 1.15 |
| 151 | 0.70 | 210 | 0.68 |
| 152 | 2.11 | 213 | 0.93 |
| 155 | 2.63 | 215 | 1.18 |
| 157 | 3.34 | 216 | 1.28 |
| 162 | 2.05 | 217 | 0.70 |
| 163 | 4.37 | 222 | 0.60 |
| 164 | 0.90 | 224 | 1.60 |
| 168 | 3.14 | 225 | 1.19 |
| 169 | 3.69 | Pirtobrutinib | 0.03 |
| 170 | 2.54 | Vecabrutinib | 0.01 |
| 172 | 2.35 | Fenebrutinib | 0.19 |

TABLE 2e
Binding Kinetics (SPR measurements) Target residence
times (τ (h)) for BTK T474S

| | Tau (h) BTK | | Tau (h) BTK |
|---|---|---|---|
| Example | T474S (Act) | Example | T474S (Act) |
| 6 | 4.41 | 85b | 4.36 |
| 7 | 4.98 | 98b | 4.46 |
| 8 | 3.17 | 115 | 3.89 |
| 9 | 1.53 | 130 | 1.64 |
| 10 | 9.46 | 131 | 9.61 |
| 11 | 4.30 | 132 | 3.04 |
| 17 | 8.71 | 133b | 3.12 |
| 18 | 5.15 | 139 | 2.11 |
| 20 | 2.75 | 141 | 4.17 |
| 21 | 3.59 | 146 | 2.69 |
| 24 | 3.19 | 147 | 6.36 |
| 25 | 2.64 | 150 | 1.50 |
| 26 | 1.79 | 151 | 2.11 |
| 27 | 4.09 | 152 | 1.24 |
| 28 | 3.77 | 153 | 1.97 |
| 33 | 5.78 | 155 | 3.80 |
| 34 | 8.10 | 156 | 1.06 |
| 39 | 5.96 | 157 | 1.47 |
| 40 | 8.38 | 162 | 2.02 |
| 44 | 3.27 | 163 | 6.74 |
| 45 | 2.94 | 164 | 6.88 |
| 46 | 4.80 | 168 | >24 |
| 47 | 2.76 | 169 | 2.56 |
| 48 | 4.18 | 170 | 3.15 |
| 49 | 4.63 | 172 | 18.95 |
| 51 | 2.09 | 175 | 1.14 |
| 52 | 1.35 | 176 | 1.95 |
| 53 | 1.26 | 179 | 3.01 |
| 54 | 4.37 | 180 | 7.27 |
| 56 | 2.62 | 181 | 5.65 |
| 57 | 1.96 | 182 | 1.03 |
| 61 | 2.48 | 183 | >24 |
| 68 | 3.79 | 187 | 13.64 |
| 71 | 4.32 | Pirtobrutinib | 1.25 |
| 76 | 1.85 | Vecabrutinib | 0.07 |
| 79 | 6.20 | Fenebrutinib | 0.93 |
| 83 | 3.04 | | |

### Example F

In vitro cellular (mutant) BTK inhibition, and inhibitor wash-out
Generation of human wt-BTK, BTK C481S, BTK T474I, and BTK C481S/T474I expressing GripTite 293 MSR cells.

GripTite 293 MSR cells (Thermo Fisher, cat. no. R79507), here after referred to as 293 cells, were cultured in DMEM/F-12, GIutaMAX™ supplement medium (Thermo Fisher cat. no. 10565018) supplemented with 1% Penicilline/Streptomycine (Thermo Fisher cat. no. 15140122) 10% fetal bovine serum (Biowest, cat. no. Si181 0-500), 1× MEM Non-Essential Amino Acids (Thermo Fisher cat. no. 11140050) and 50 μg/ml Geneticin (Thermo Fisher cat. no. 10131035). Cells were transfected, with pEF6/V5-HisB (Thermo Fisher cat. no. V96120) expression vectors containing either full-length wildtype-BTK (canonical sequence NM_000061), BTK C481 S, BTK T474I or BTK C481 S/T474I (BaseClear) using Lipofectamine™ 3000 (Invitrogen). Because the stop codon at the end of the coding sequence of BTK has been conserved, the His-tag present in the expression vector is not being used. Immediately after transfection, cells were cultured in medium without Geneticin, and after 24 hours 50 μg/ml geneticin was added. 48 hours after transfection, medium was removed and replaced by medium containing both geneticin (50 μg/ml) and blasticidin (10 μg/ml) S HCl (Thermo Fisher, cat. no. A1113903). The resulting transfectants were cultured under blasticidin selection pressure for 18-35 passages to obtain stable wild-type BTK (wt-BTK), BTK C481S, BTK T474I, and BTK C481 S/T474I expressing cell pools. Stability of the (mutant) BTK expression in the 293 cell pools was confirmed by poly-acrylamide gel electrophoresis followed by Western blotting (FIG. 1A). Non-transfected 293 cells do not express BTK (FIG. 1B). For both experiments 10 μg of protein was separated on 4-12% Bis-Tris polyacrylamide gels.

PAGE & Western blotting.

Wild-type BTK, BTK C481S, BTK T474I or BTK C481 S/T474I expressing 293 cells were lysed in lysis buffer supplemented with protease inhibitors (Merck, cat. no. P8340) and phosphatase inhibitors (Thermo Fisher, cat. no. 78426). The protein concentration was determined with a Pierce BCA protein assay kit (Thermo Fisher, cat. no. 23227). Lysates were diluted to a concentration of 1 μg/μl in NuPage® LDS Sample Buffer (Thermo Fisher, cat. no. NP0008) with 167 mM DTT (Acros Organics, cat. no. 10686841) and denatured for 5 minutes at 95° C. The samples (10 μg protein) were, together with a pre stained protein ladder (Thermo Fisher, cat. no. 26616), separated on 4-12% Bis-Tris polyacrylamide gels (Thermo Fisher, cat. no. NP0324BOX). Separated protein was subsequently transferred, in methanol free transfer buffer (Thermo Fisher, cat. no. 35045), to a nitrocellulose membrane (Thermo Fisher, cat. no. 88018). Blots were first immunologically stained with phospho-BTK (Tyr223) rabbit mAb (Cell Signaling, cat. no. 5082S), and beta-actin rabbit mAb (Cell Signaling, cat. no. 4967S), followed by peroxidase conjugated goat anti-rabbit IgG (Cell Signaling, cat. no. 7074S). Afterwards, antibodies were removed using Western-blot stripping buffer (Thermo Fisher cat. no. 46428) and staining was repeated with a (total-)BTK mouse mAb (Abnova, cat. no. MAB0798), followed by peroxidase conjugated horse anti-mouse IgG (Cell Signaling, cat. no. 7076S). For all blots the colour development was performed with ECL horseradish peroxidase substrate (BioRad, cat. no. 170-5060).

Cell based BTK inhibition.

Wild-type BTK, BTK C481S, BTK T474I, and BTK C481 S/T474I expressing 293 cells were growing exponentially when seeded. Cells were seeded in culture medium without Blasticidin, at a density of 800,000 cells/well in 6 well plates (Greiner cell star, cat no 657160), and placed in a 37° C. CO₂-incubator. Between 16 and 24 hours after seeding, medium with compound of Example 46 (leading to a final concentration ranging from 0.1 to 1000 nM) or medium with the compound of Example 25 (leading to a final concentration ranging from 0.1 to 1000 nM) or medium with the compound of Example 26 (leading to a final concentration ranging from 0.1 to 1000 nM) or medium with the compound of Example 83 (leading to a final concentration ranging from 0.1 to 1000 nM) or medium with the compound of Example 68 (leading to a final concentration ranging from 0.1 to 1000 nM) or medium with the compound of Example 45 (leading to a final concentration ranging from 0.1 to 1000 nM) or medium with the compound of (+/−) Loxo-305 (leading to a final concentration ranging from 0.1 to 1000 nM) or 0.05% DMSO (no compound) was added to the cells. Twenty-four hours after addition of the inhibitors, cells were detached from the well using 0.25% trypsin (Thermo Fisher, cat. no. 25200056). After collection cells were washed twice with PBS (Thermo Fisher, cat. no. 14190-094), cell pellets were snap-frozen, and stored at −80° C. 24h hours after addition of the compounds cells were harvested and the level of BTK phosphorylation (pBTK (Tyr223)) was determined on Western blot (FIG. 2A, 2B, 2C, 2D, 2E, 2F and 2G). Beta-actin (ACTB) and BTK (total BTK) levels were determined as loading and BTK expression control (FIG. 2A, 2B, 2C, 2D, 2E, 2F and 2G).

Cell based washout.

Wild-type BTK, BTK C481S, BTK T474I, and BTK C481 S/T474I expressing 293 cells were growing exponentially when seeded in culture medium, without blasticidin, at a density of 2,000,000 cells/4 ml/well, in 25 cm² cell culture flasks (Greiner Bio-one, cat. no. 690175). Flasks were placed in a 37° C. C02-incubator for 16-24 hours after which 1 ml culture medium containing 5000 nM of the compound of Example 46 or Example 201, or Example 184, or Example 204, or Example 45 or (+/−) Loxo-305, or 0.05% DMSO (no compound) was added. 2 hours after addition of the inhibitors (0h) cells are either collected or washed twice with 5 ml culture medium. After washing, 5 ml culture medium (without blasticidin), was added and cells were incubated at 37° C. in a CO2 incubator for 0.5, 1, 2, 4, 6, or 24 hours before being collected. Cells were harvested using 0.25% trypsin (Thermo Fisher, cat. no. 25200056). After harvesting, cells pellets were washed twice with PBS (Thermo Fisher, cat. no. 14190-094), snap-frozen, and stored at −80° C.

Wash-out Western blot results of wild-type BTK, BTK C481S, BTK T474I, and BTK C481 S/T474I expressing 293 cells are shown for the compound of Example 46 in FIG. 3A and FIG. 3H, for (+/−) Loxo-305 in FIG. 3B and FIG. 3F (Pirtobrutinib is Loxo-305), for compound of Example 201 in FIG. 3C and FIG. 3F, for compound of Example 184 in FIG. 3D and FIG. 3F, for compound of Example 204 in FIG. 3E and FIG. 3F and for compound of Example 45 in FIG. 3G and FIG. 3H. Control cells (no compound) were incubated for two hours with 0.01% DMSO (D) and harvested at 0 hours (0h), which is at the start of the was-out, and 24 hours (24h). The level of phosphorylated BTK (pBTK (Tyr223)) was determined on Western blot. Beta-actin (ACTB) and BTK (total BTK) levels were determined as loading control and BTK expression control.

It is clear from the wash-out experiments that the reference compound Loxo-305 does not prohibit or reduce the level of phosphorylated BTK (pBTK (Tyr223)), while the compounds of Example 46, Example 25, Example 201, Example 184, Example 204, and Example 45 all prohibit or reduce the level of phosphorylated BTK (pBTK (Tyr223)) for at least 6 hours up till at least 24 hours, even for cell lines expressing mutant variations of BTK. This shows that the target residence of the compounds according to the invention is still substantial to complete during said period after removing the solution of compounds.

### Example G

Cell proliferation assay wt-TMD8 diffuse large B-cell lymphoma cells were purchased from Tokyo Medical and Dental University and cultured in RPMI-1640 cell culture medium (cat. no. 61870036, Life Technologies), supplemented with 10% (v/v) heat-inactivated fetal bovine calf serum and 1% penicillin/streptavidin. 1600 cells per well (in 45 μl) were seeded in a white 384-well culture plate (cat. no. 781080, Greiner Bio-One) and allowed to rest for at least 5 hours at 37° C., 95% humidity, and 5% CO₂. 5 μl compound solution was added to the cells and incubation was continued for 120 hours (5 days), followed by addition of 24 μl ATPlite 1Step™ (PerkinElmer, Groningen, The Netherlands) solution was added to each well. Luminescence was recorded on an Envision multimode reader. The cell signal at the start of incubation was recorded separately in order to distinguish between cell population growth and cell death. In addition, maximum growth was determined by incubation of a duplicate without compound in the presence of 0.3% DMSO. Percentage growth was used as the main y-axis signal. IC₅₀ₛ were fitted by non-linear regression using IDBS XLfit™5 using a 4-parameter logistic curve, yielding a maximum signal, minimum signal, hill-parameter and IC₅₀.
Results of the wt-TMD8 proliferation are shown in Table 3 below.
IC₅₀ wt-TMD8: A means IC₅₀<100 nM
B means IC₅₀ between 100 nM and 1 μM
C means IC₅₀>1 μM and <10 μM

TABLE 3
IC₅₀ values for wt-TMD8 cellular assays.

| | IC50 |
|---|---|
| Exam- | wt-TMD8 |
| ple | (nM) |
| 1 | A |
| 2 | A |
| 3 | A |
| 4 | B |
| 5 | A |
| 6 | A |
| 7 | A |
| 8 | A |
| 9 | A |
| 10 | A |
| 11 | A |
| 12 | A |
| 13 | A |
| 14 | A |
| 15 | A |
| 16 | A |
| 17 | A |
| 18 | A |
| 20 | A |
| 21 | A |
| 23 | A |
| 24 | A |
| 25 | A |
| 26 | A |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | A |
| 39 | A |
| 40 | A |
| 41 | A |
| 42 | A |
| 43 | A |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 55 | A |
| 56 | A |
| 57 | A |
| 59 | A |
| 60 | A |
| 61 | A |
| 68 | A |
| 69 | A |
| 70 | A |
| 71 | A |
| 72 | A |
| 73 | A |
| 74 | A |
| 75 | A |
| 76 | A |
| 77 | B |
| 78 | A |
| 79 | A |
| 80 | A |
| 81 | A |
| 82 | A |
| 83 | A |
| 85b | A |
| 86 | B |
| 87b | A |
| 88b | A |
| 94 | C |
| 97b | A |
| 99b | A |
| 100b | A |
| 101b | A |
| 102b | A |
| 103b | A |
| 104b | B |
| 105 | B |
| 114 | A |
| 118 | A |
| 119 | A |
| 123 | A |
| 128 | A |
| 129 | A |
| 130 | A |
| 131 | A |
| 133b | A |
| 137 | A |
| 138 | A |
| 139 | A |
| 140 | A |
| 141 | A |
| 142 | A |
| 143 | A |
| 144 | A |
| 145 | A |
| 146 | A |
| 147 | A |
| 148 | A |
| 149 | A |
| 150 | A |
| 151 | A |
| 152 | A |
| 153 | A |
| 154 | A |
| 155 | A |
| 156 | A |
| 157 | A |
| 158 | A |
| 159 | A |
| 160 | A |
| 161 | A |
| 162 | A |
| 163 | A |
| 164 | A |
| 165 | A |
| 166 | A |
| 167 | A |
| 168 | A |
| 169 | A |
| 170 | A |
| 171 | C |
| 172 | A |
| 173 | A |
| 174 | A |
| 175 | A |
| 176 | A |
| 177 | A |
| 178 | A |
| 179 | A |
| 180 | A |
| 181 | A |
| 182 | A |
| 183 | A |
| 184 | A |
| 185 | A |
| 186 | A |
| 187 | A |
| 188 | A |
| 189 | A |
| 190 | A |
| 191 | A |
| 192 | A |
| 193 | A |
| 194 | A |
| 195 | A |
| 196 | A |
| 197 | A |
| 198 | A |
| 199 | A |
| 200 | A |
| 201 | A |
| 202 | A |
| 203 | A |
| 204 | A |
| 205 | A |
| 206 | A |
| 207 | A |
| 208 | A |
| 209 | A |
| 210 | A |
| 211 | A |
| 212 | A |
| 213 | A |
| 214 | A |
| 215 | A |
| 216 | A |
| 217 | A |
| 218 | A |
| 219 | A |
| 220 | A |
| 221 | A |
| 222 | A |
| 223 | A |
| 224 | A |

### REFERENCES

Attwood et al. (2021) “Trends in kinase drug discovery targets, indications and inhibitor design.” Nat Rev Drug Discov 20, pages 839-861 (2021).
Xianhui Wang et al. (2021) Bruton's Tyrosine Kinase and Its Isoforms in Cancer. Front. Cell Dev. Biol. 9:668996
Kokabee L. et al., Bruton's Tyrosine kinase is a potential therapeutic target in prostate cancer cells (2015) Cancer Biology & Therapy 16;11, 1604-1615
Wang et al, Bruton's Tyrosine Kinase inhibitors prevent therapeutic escape in breast cancer cells; Mol Cancer Ther. (2016) 15(9) 2198-2208
Wei et al. (2016) Oncotarget 7(43), 69961-69975
Wang et al. (2017) J Exp Clin Cancer Res, 36, pp96
Stefan F. H. Neys et al., Targeting Bruton's Tyrosine Kinase in inflammatory and autoimmune pathalogies;(2021) Front Cell Dev Biol, 9:668131
Rula Zain et al., Structure-Function relationships of covalent and non-covalent BTK inhibitors (2021) Front Immunol 12:694853
Wang et al. (2019) Noncovalent inhibitors reveal BTK gatekeeper and auto-inhibitory residues that control its transforming activity: JCI Insight. 2019;4(12):e127566.
Cohen et al. 2021, Kinase drug discovery 20 years after imatinib: progress and future directions; Nature Reviews Drug Discovery volume 20, pages 551-569 (2021))
Willemsen-Seegers N. et al (2016) J Mol Biol (2017) 429, 574-586
Copeland R. A. et al (2006) Nat Rev Drug Discov, 5, 730
Barf T. and Kaptein A. (2012) J Med Chem, 55(14):6243-62
Willemsen-Seegers N. et al (2016) J Mol Biol (2017) 429, 574-586
Driggers, E M et al (2008) Nature Reviews Drug Discovery, 7(7), 608-624
Mallinson J et al (2012) Future Med. Chem. 4(11), 1409-1438; Macrocycles in new drug discovery
P. J. Tummino, R.A. Copeland, Residence time of receptor-ligand complexes and its effect on biological function, Biochemistry 47 (2008) 5481-5492.

1-31. (canceled)

32. A compound of Formula (I-a) to (I-h) or a pharmaceutically acceptable salt and/or solvate thereof, wherein the compound is selected from the group consisting of: 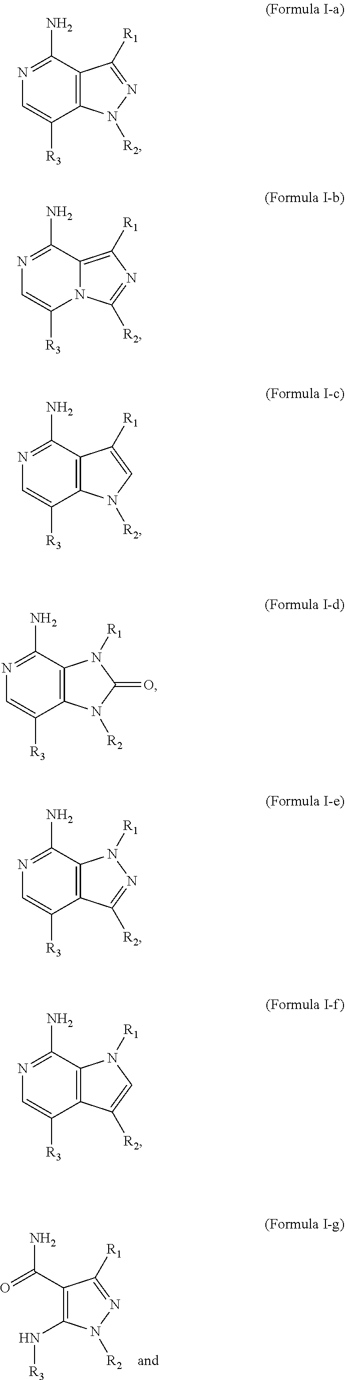 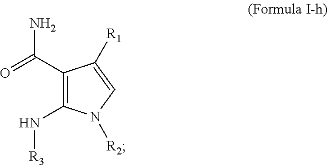
Wherein R¹ is 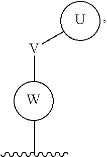
wherein:
W is an aryl group having 6-10 carbon or a heteroaryl group having 1-5 carbon; wherein any said aryl group and heteroaryl group is optionally and independently substituted with one or more substituents selected from halogen, (1-2C)alkyl, (1-2C)alkoxy; wherein any of said alkyl and alkoxy group is optionally and independently substituted with one, two or three fluoro;
V is any one of O, —C(O)—NH—, —NH—C(O)—, —CH(R^{1v})—NH—C(O)—, —CH(R^{1v})—;
R^{1v} is hydrogen or (1-2C)alkyl;
U is an aryl group having 6-10 carbon or an heteroaryl group having 1-5 carbon; wherein any of said aryl group and heteroaryl group is optionally and independently substituted with one or more substituents selected from halogen, cyano, (1-4C)alkyl, (1-5C)alkoxy, (3-6C)cycloalkyl or (3-6C)heterocycloalkyl; wherein any of said alkyl, alkoxy, cycloalkyl and heterocycloalkyl group is optionally and independently substituted with one, two or three halogen;
wherein R² is of Formula (II-a) to (II-f) selected from the group consisting of: 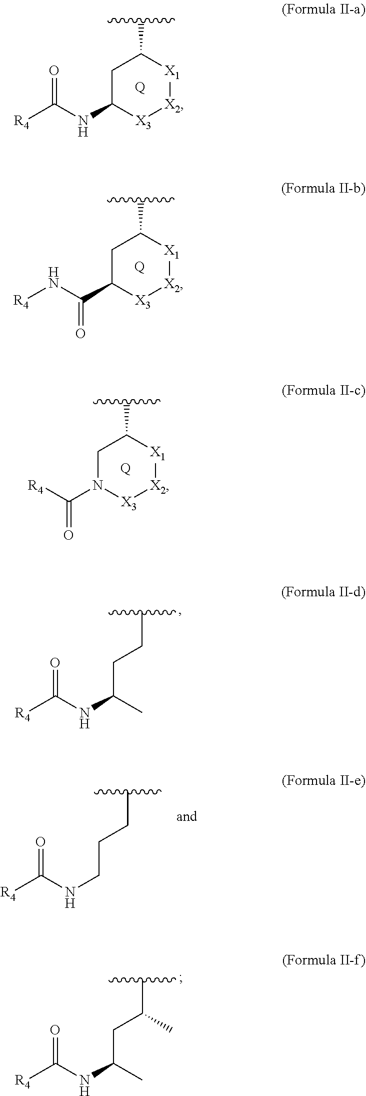
wherein Q is a monocyclic ring selected from a (3-7C)cycloalkyl and a (3-6C)heterocycloalkyl, wherein X₁, X₂ and X₃ are independently selected from CH₂, —CH₂CH₂—, O, N and a direct bond; wherein any of the cycloalkyl, heterocycloalkyl and alkyl group is optionally and independently substituted with one or more substituents selected from halogen, hydroxy, (1-3C)alkyl, (1-3C)alkoxy, (1-4C)alkylcarbonyl or (3-4C)cycloalkyl; wherein any of said alkyl and alkoxy group is optionally and independently substituted with one, two or three halogen;
wherein R³ and R⁴ together represent a linker having Formula (III-1 to III-40) selected from the group consisting of: 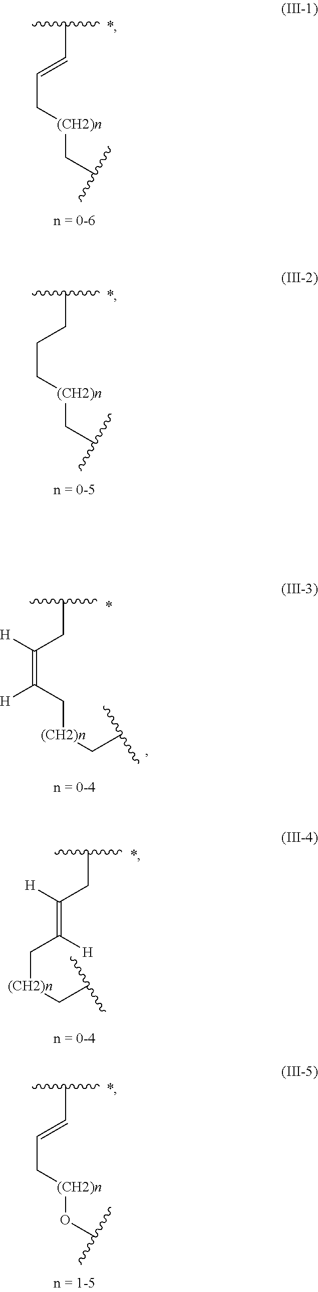 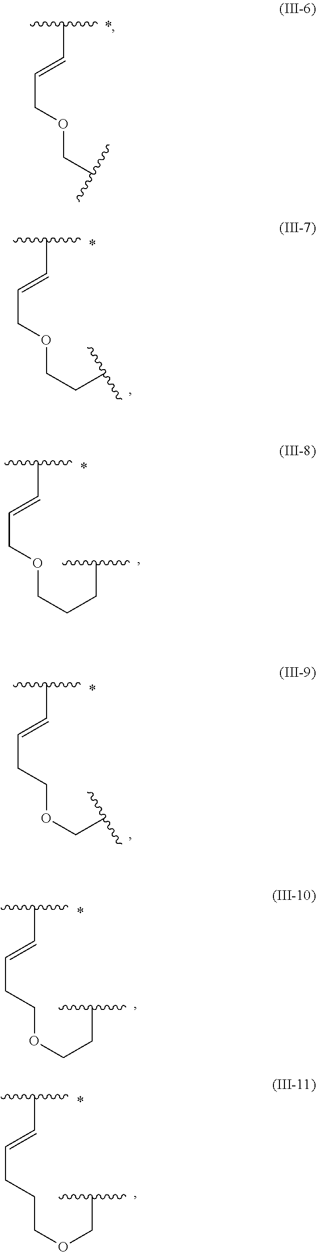 [SMILES: */C=C/COC* ; */C=C/COCC* ; */C=C/COCCC* ; */C=C/CCOC* ; */C=C/CCOCC* ; */C=C/CCCOC*] 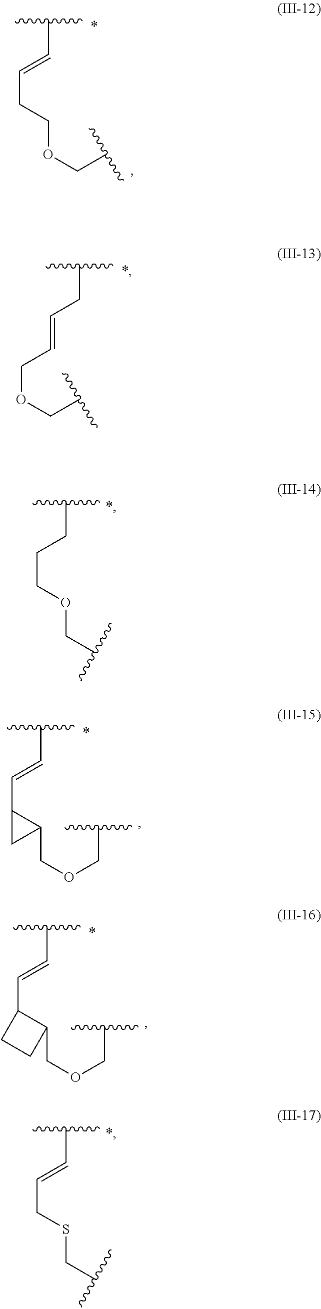 [SMILES: */C=C/CCOC* ; *C/C=C/COC* ; *CCCOC* ; */C=C/C1CC1COC* ; */C=C/C1CCC1COC* ; */C=C/CSC*] 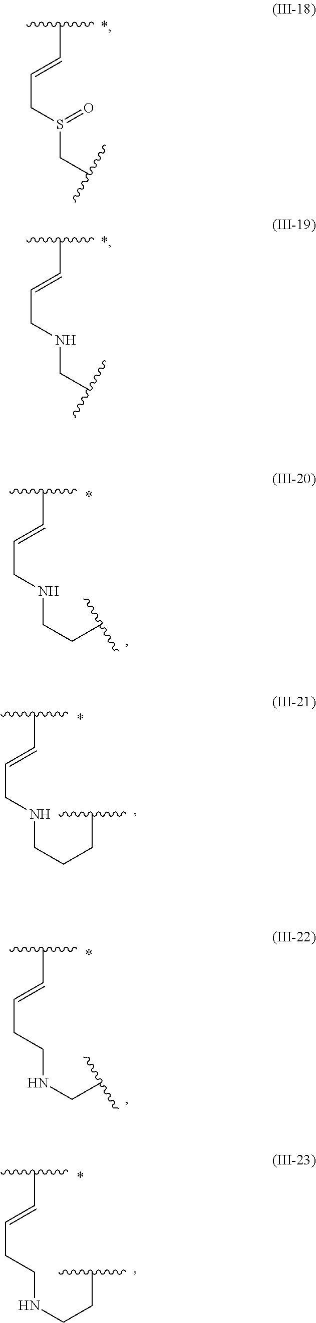 [SMILES: */C=C/CS(=O)C* ; */C=C/CNC* ; */C=C/CNCC* ; */C=C/CNCCC* ; */C=C/CCNC* ; */C=C/CCNCC*] 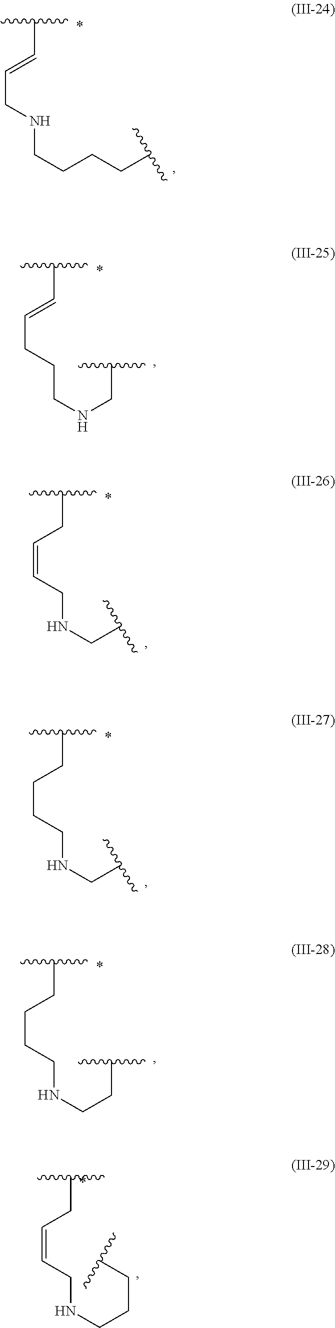 [SMILES: */C=C/CNCCCC* ; */C=C/CCCNC* ; *C/C=C\CNC* ; *CCCCNC* ; *CCCCNCC* ; *C/C=C\CNCCC*] 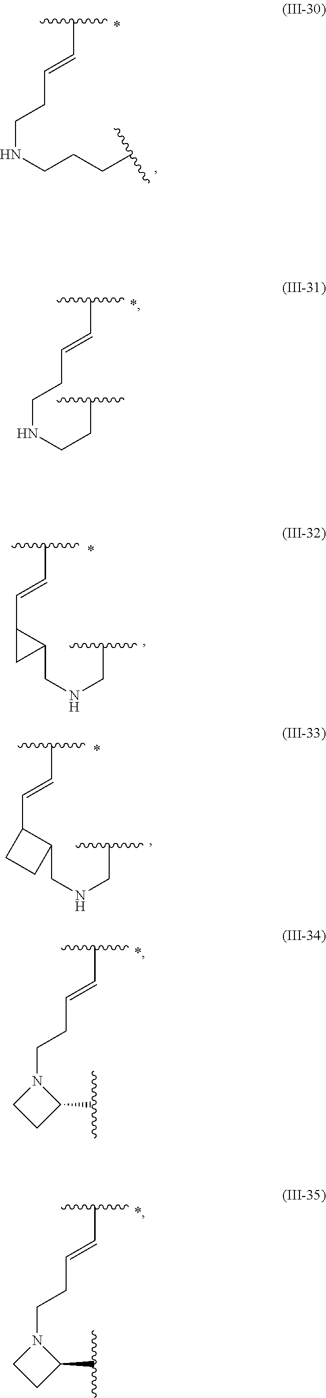 [SMILES: */C=C/CCNCCC* ; */C=C/CCNCC* ; */C=C/C1CC1CNC* ; */C=C/C1CCC1CNC* ; */C=C/CCN1CC[C@H]1* ; */C=C/CCN1CC[C@@H]1*] 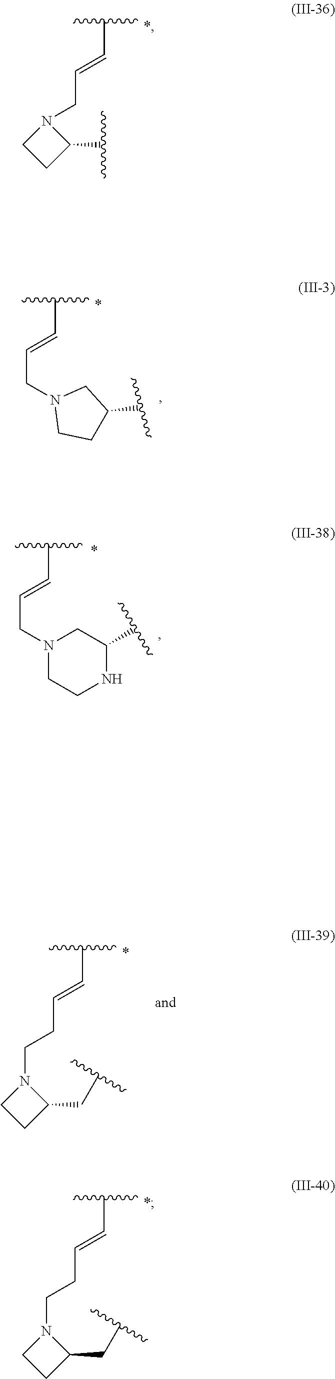 [SMILES: */C=C/CN1CC[C@H]1* ; */C=C/CN1CC[C@@H](*)C1 ; */C=C/CN1CCN[C@@H](*)C1 ; */C=C/CCN1CC[C@H]1C* ; */C=C/CCN1CC[C@@H]1C*]
whereby the 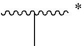
marks the position of R³ in any one of Formula I-a to I-h, and whereby the 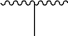
marks the position of R⁴ in any one of Formula II-a to II-f; wherein any of said linkers is optionally and independently substituted with one or more substituents selected from deuterium, halogen, oxo, hydroxy, CD₃, (1-4C)alkyl, (1-5C)alkoxy, (3-6C)cycloalkyl, (3-6C)cycloalkoxy and (1-6C)alkylcarbonyl; wherein any of said alkyl and alkoxy group is optionally and independently substituted with one, two or three halogen.

33. The compound according to claim 32, wherein the linker represented by R³ and R⁴ is selected from the group consisting of: 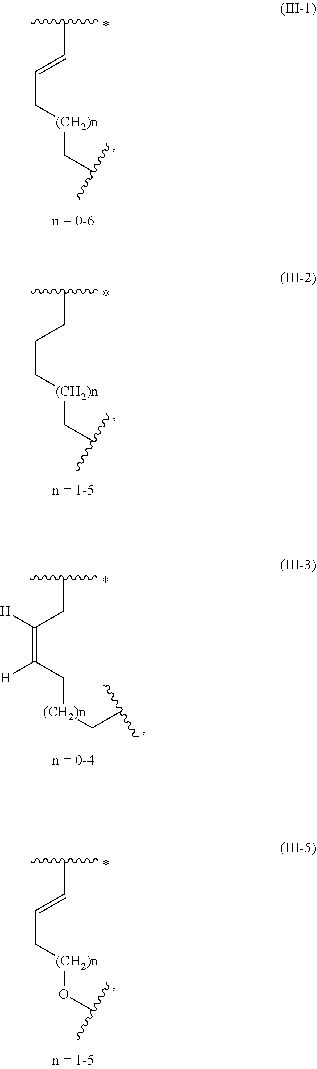 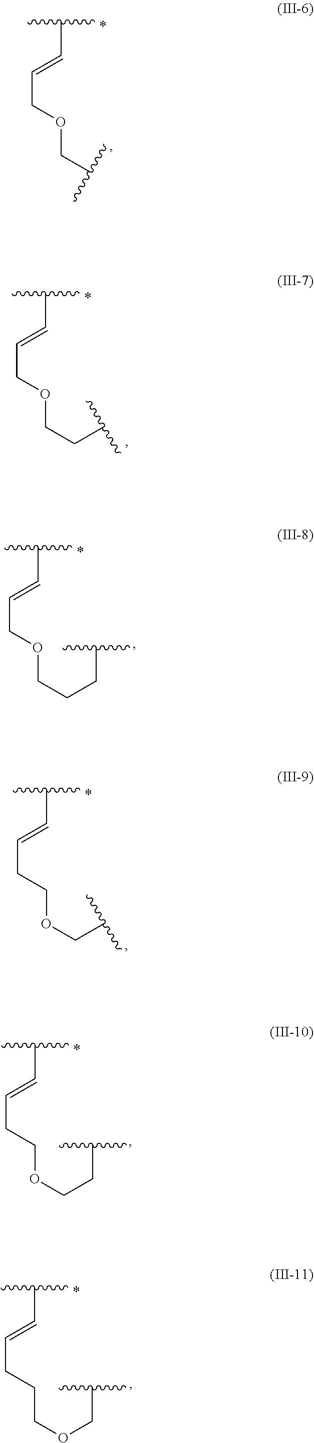 [SMILES: */C=C/COC* ; */C=C/COCC* ; */C=C/COCCC* ; */C=C/CCOC* ; */C=C/CCOCC* ; */C=C/CCCOC*] 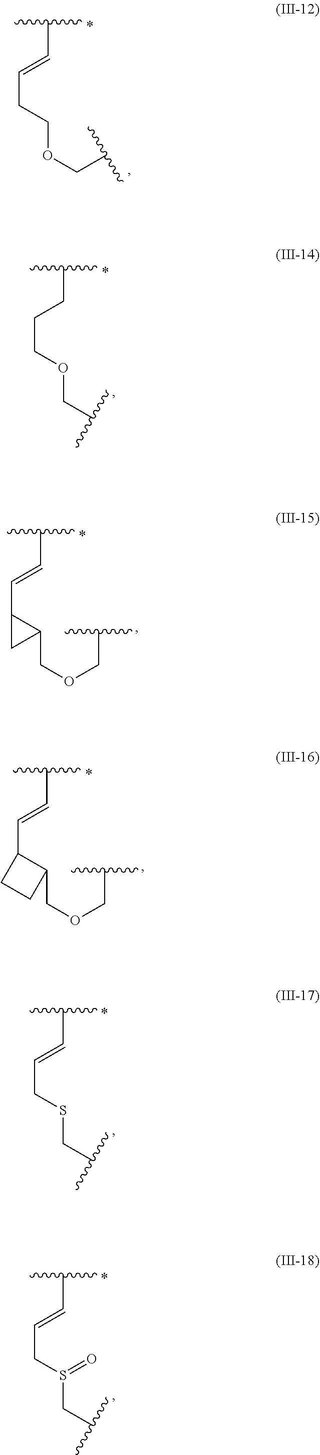 [SMILES: */C=C/CCOC* ; *CCCOC* ; */C=C/C1CC1COC* ; */C=C/C1CCC1COC* ; */C=C/CSC* ; */C=C/CS(=O)C*] 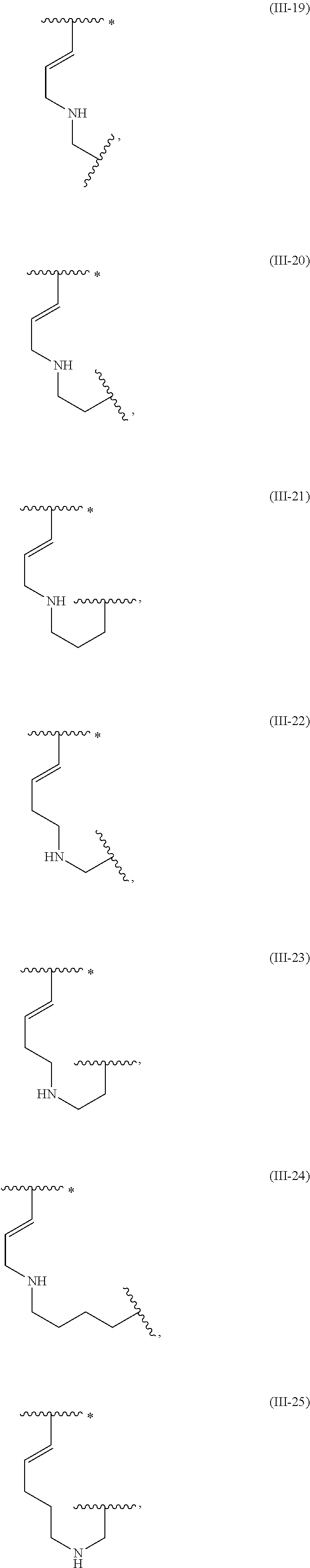 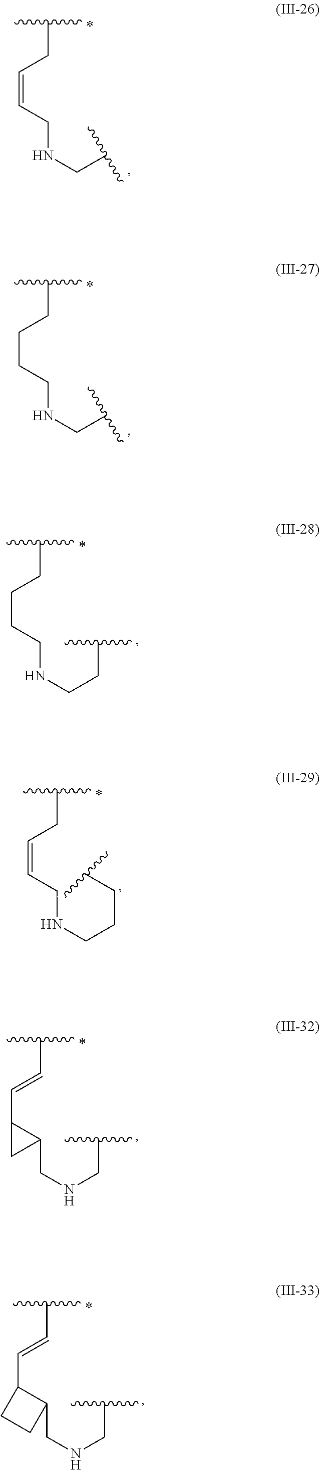 [SMILES: *C/C=C\CNC* ; *CCCCNC* ; *CCCCNCC* ; *C/C=C\CNCCC* ; */C=C/C1CC1CNC* ; */C=C/C1CCC1CNC*] 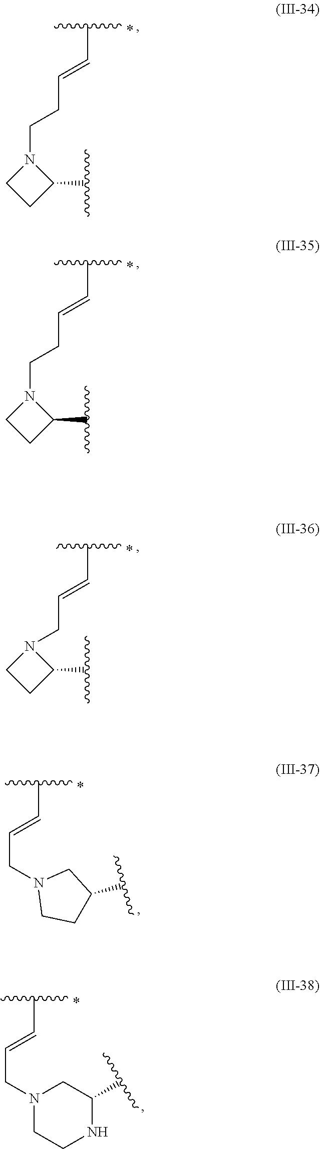 [SMILES: */C=C/CCN1CC[C@H]1* ; */C=C/CCN1CC[C@@H]1* ; */C=C/CN1CC[C@H]1* ; */C=C/CN1CC[C@@H](*)C1 ; */C=C/CN1CCN[C@@H](*)C1] 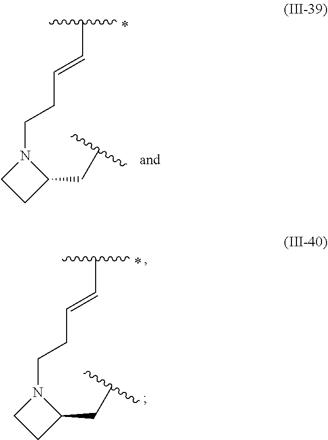 [SMILES: */C=C/CCN1CC[C@H]1C* ; */C=C/CCN1CC[C@@H]1C*]
whereby the 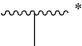
marks the position of R³ in any one of Formula I-a to I-h, and
whereby the 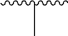
marks the position of R⁴ in any one of Formula II-a to II-f
wherein any of said linkers is optionally and independently substituted with one or more substituents selected from deuterium, halogen, oxo, hydroxy, CD₃, (1-4C)alkyl, (1-5C)alkoxy, (3-6C)cycloalkyl, (3-6C)cycloalkoxy and (1-6C)alkylcarbonyl; wherein any of said alkyl and alkoxy group is optionally and independently substituted with one, two or three halogen.

34. The compound according to claim 32, wherein R² is selected from the group consisting of: 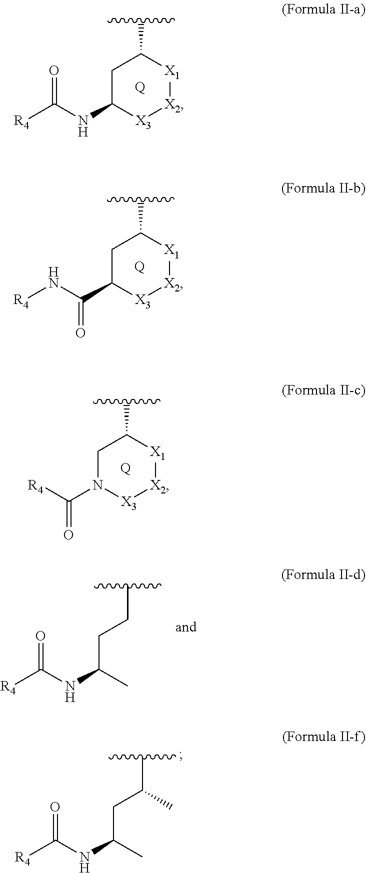
wherein Q is a monocyclic ring selected from a (3-7C)cycloalkyl and a (3-6C)heterocycloalkyl,
wherein X₁, X₂ and X₃ are independently selected from CH₂, —CH₂CH₂, —O, N and a direct bond;
wherein any of said cycloalkyl, heterocycloalkyl and alkyl group is optionally and independently substituted with halogen, hydroxy, (1-3C)alkyl, (1-3C)alkoxy, (1-4C)alkylcarbonyl or (3-4C)cycloalkyl; wherein any of said alkyl and alkoxy group is optionally and independently substituted with one, two or three halogen.

35. The compound according to claim 32, wherein the compound comprises a bicyclic scaffold selected from:

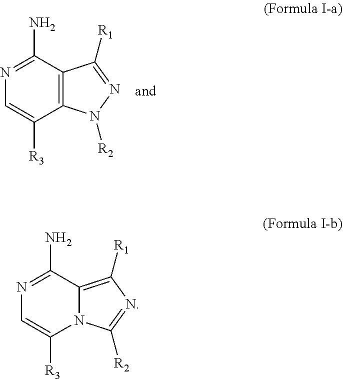

36. The compound according to claim 32, wherein R¹ is:
selected from the group consisting of: 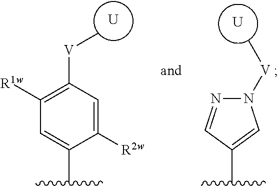
wherein R^{1w} and R^{2w} are independently selected from hydrogen, halogen, (1-2C)alkyl, (1-2C)alkoxy; wherein any of said alkyl and alkoxy group is optionally and independently substituted with one, two or three fluoro; or
any of: 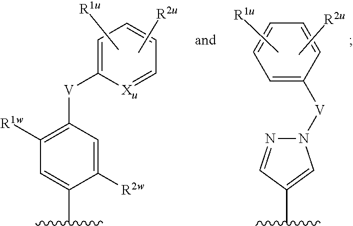
wherein R^{1w} and R^{2w} are independently selected from hydrogen, halogen, (1-2C)alkyl, (1-2C)alkoxy; wherein any of said alkyl and alkoxy group is optionally and independently substituted with one, two or three fluoro;
wherein R^{1u} and R^{2u} are independently selected from hydrogen, halogen, cyano, (1-4C)alkyl, (1-5C)alkoxy, (3-6C)cycloalkyl or (3-6C)heterocycloalkyl; wherein any of said alkyl and alkoxy group is optionally and independently substituted with one, two or three halogen;
wherein X^{u} is selected from CH and N;
wherein R¹ is any one of: 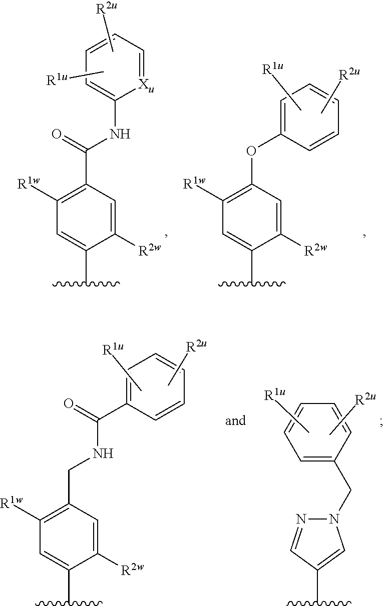
wherein R^{1u} and R^{2w} are independently selected from hydrogen, halogen, (1-2C)alkyl, (1-2C)alkoxy; wherein any of said alkyl or alkoxy group is optionally and independently substituted with one, two or three fluoro;
wherein R^{1u} and R^{2u} are independently selected from hydrogen, halogen, cyano, (1-4C)alkyl, (1-5C)alkoxy, (3-6C)cycloalkyl or (3-6C)heterocycloalkyl; wherein any of said alkyl and alkoxy group is optionally and independently substituted with one, two or three halogen;
wherein X^{u} is selected from CH and N;
any one of: 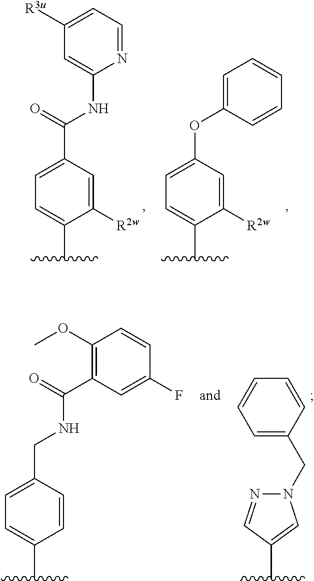
wherein R^{2w} is selected from hydrogen, halogen, (1-2C)alkyl, (1-2C)alkoxy; wherein any said alkyl or alkoxy group is optionally and independently substituted with one, two or three fluoro;
wherein R^{3u} is selected from hydrogen, halogen, cyano, (1-4C)alkyl, (1-5C)alkoxy, (3-6C)cycloalkyl or (3-6C)heterocycloalkyl; wherein any of said alkyl and alkoxy group is optionally and independently substituted with one, two or three fluoro; or
any one of: 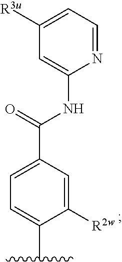
wherein R^{2w} is selected from hydrogen, fluoro, methyl or methoxy;
wherein R^{3u} is selected from hydrogen, halogen, cyano, (1-4C)alkyl, (1-2C)alkoxy, (3-6C)cycloalkyl or (3-6C)heterocycloalkyl; wherein any of said alkyl and alkoxy group is optionally and independently substituted with one, two or three fluoro.

37. The compound according to claim 32, wherein V is any one of O, —C(O)—NH—, —CH(R^{1v})—NH—C(O)—, —CH(R^{1v})—; R^{1V} is hydrogen or (1-2C)alkyl.

38. The compound according to claim 32, wherein the compound has a sub-formula 1-226 selected from the group consisting of:

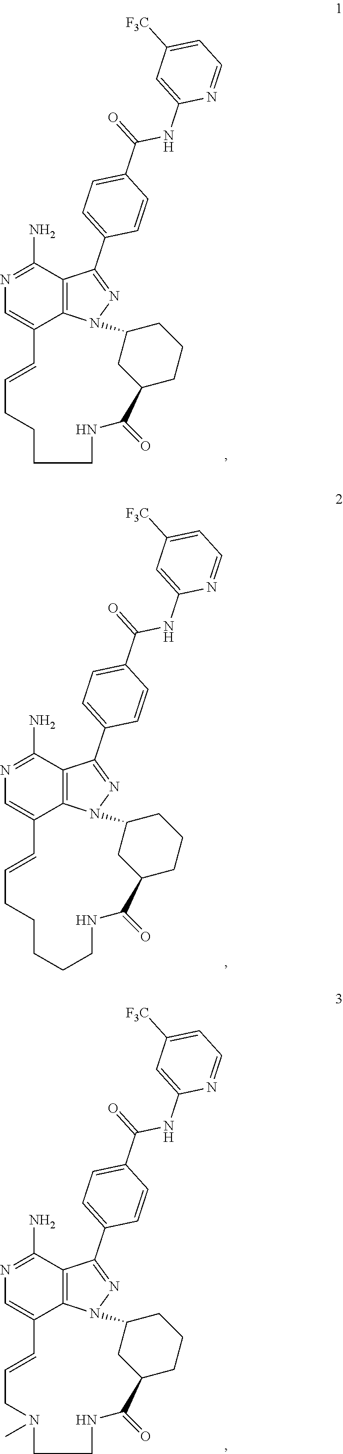 [SMILES: Nc1ncc2c3c1c(-c1ccc(C(=O)Nc4cc(C(F)(F)F)ccn4)cc1)nn3[C@@H]1CCC[C@H](C1)C(=O)NCCCC/C=C/2 ; Nc1ncc2c3c1c(-c1ccc(C(=O)Nc4cc(C(F)(F)F)ccn4)cc1)nn3[C@@H]1CCC[C@H](C1)C(=O)NCCCCC/C=C/2 ; CN1C/C=C/c2cnc(N)c3c(-c4ccc(C(=O)Nc5cc(C(F)(F)F)ccn5)cc4)nn(c23)[C@@H]2CCC[C@H](C2)C(=O)NCC1]

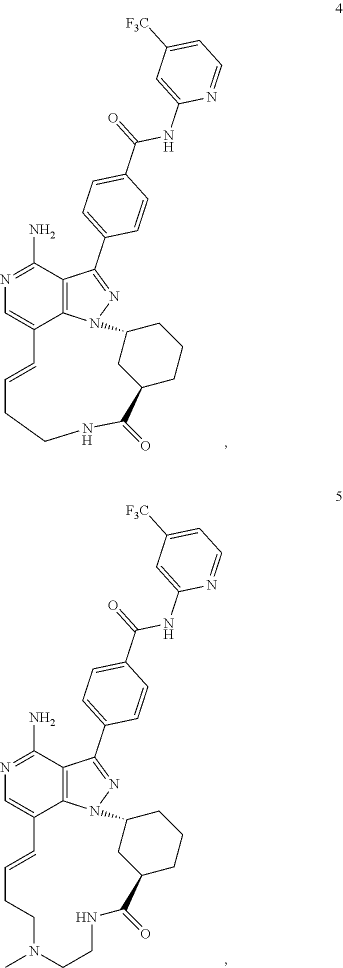

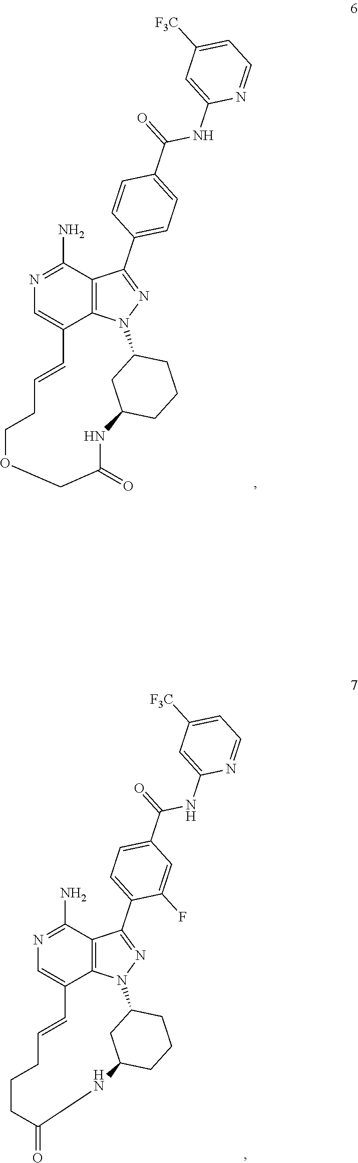 [SMILES: Nc1ncc2c3c1c(-c1ccc(C(=O)Nc4cc(C(F)(F)F)ccn4)cc1)nn3[C@@H]1CCC[C@H](C1)NC(=O)COCC/C=C/2 ; Nc1ncc2c3c1c(-c1ccc(C(=O)Nc4cc(C(F)(F)F)ccn4)cc1F)nn3[C@@H]1CCC[C@H](C1)NC(=O)CCC/C=C/2]

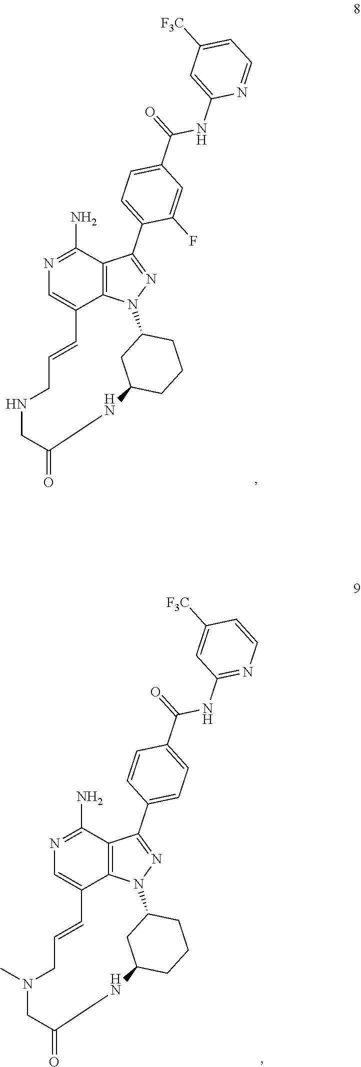 [SMILES: Nc1ncc2c3c1c(-c1ccc(C(=O)Nc4cc(C(F)(F)F)ccn4)cc1F)nn3[C@@H]1CCC[C@H](C1)NC(=O)CNC/C=C/2 ; CN1C/C=C/c2cnc(N)c3c(-c4ccc(C(=O)Nc5cc(C(F)(F)F)ccn5)cc4)nn(c23)[C@@H]2CCC[C@H](C2)NC(=O)C1]

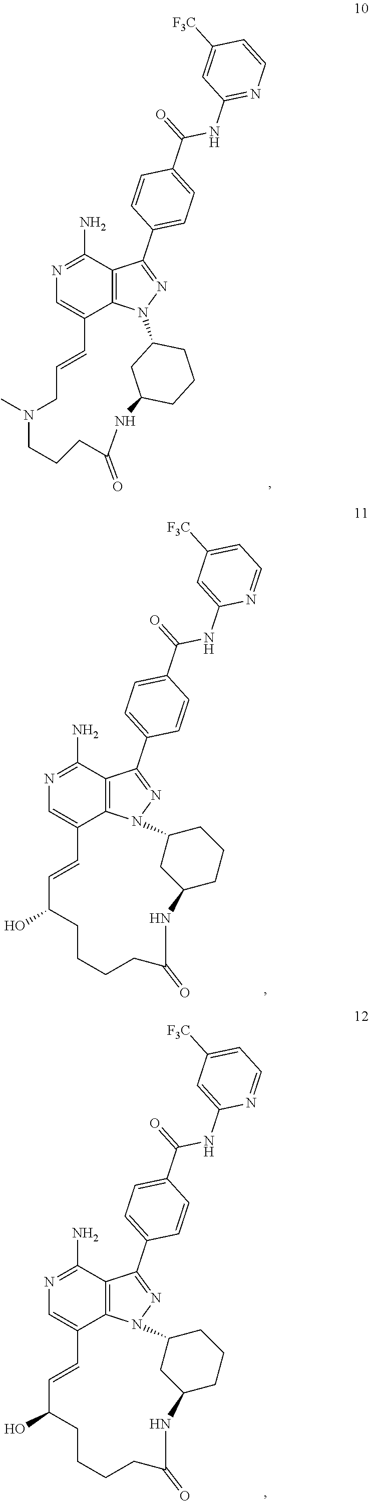

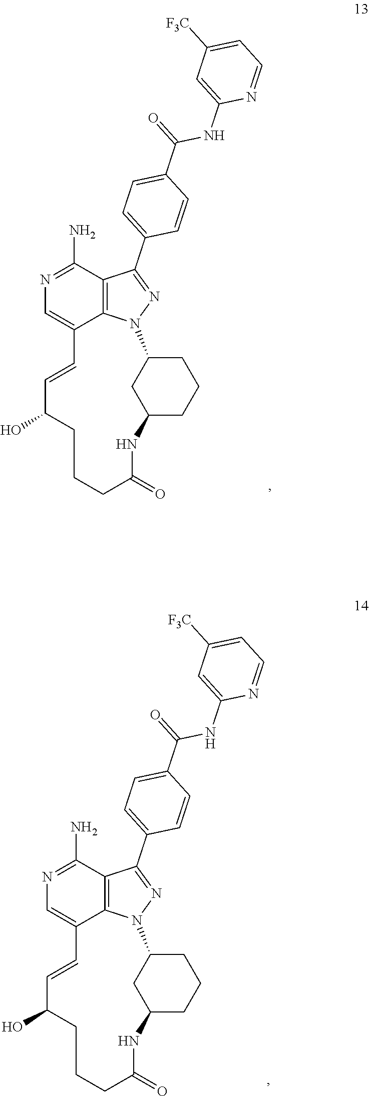 [SMILES: Nc1ncc2c3c1c(-c1ccc(C(=O)Nc4cc(C(F)(F)F)ccn4)cc1)nn3[C@@H]1CCC[C@H](C1)NC(=O)CCC[C@H](O)/C=C/2 ; Nc1ncc2c3c1c(-c1ccc(C(=O)Nc4cc(C(F)(F)F)ccn4)cc1)nn3[C@@H]1CCC[C@H](C1)NC(=O)CCC[C@@H](O)/C=C/2]

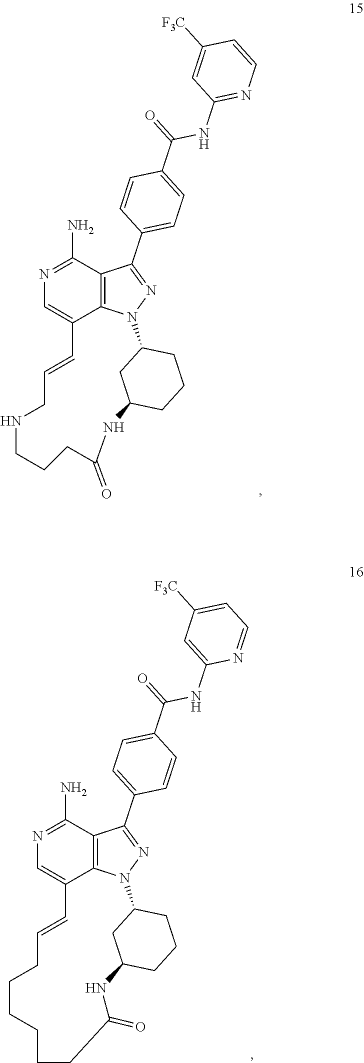 [SMILES: Nc1ncc2c3c1c(-c1ccc(C(=O)Nc4cc(C(F)(F)F)ccn4)cc1)nn3[C@@H]1CCC[C@H](C1)NC(=O)CCCNC/C=C/2 ; Nc1ncc2c3c1c(-c1ccc(C(=O)Nc4cc(C(F)(F)F)ccn4)cc1)nn3[C@@H]1CCC[C@H](C1)NC(=O)CCCCCC/C=C/2]

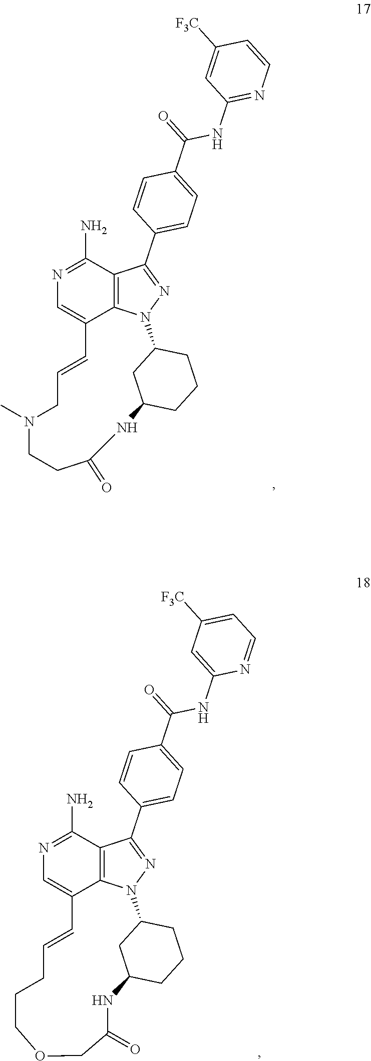 [SMILES: CN1C/C=C/c2cnc(N)c3c(-c4ccc(C(=O)Nc5cc(C(F)(F)F)ccn5)cc4)nn(c23)[C@@H]2CCC[C@H](C2)NC(=O)CC1 ; Nc1ncc2c3c1c(-c1ccc(C(=O)Nc4cc(C(F)(F)F)ccn4)cc1)nn3[C@@H]1CCC[C@H](C1)NC(=O)COCCC/C=C/2]

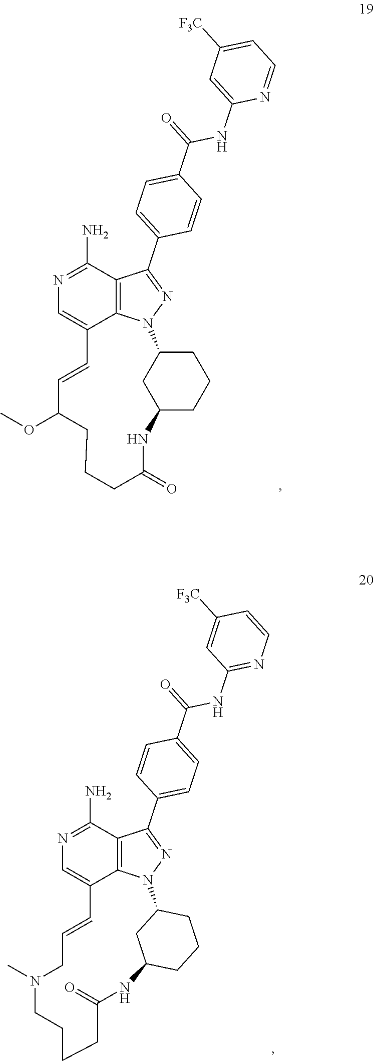 [SMILES: COC1/C=C/c2cnc(N)c3c(-c4ccc(C(=O)Nc5cc(C(F)(F)F)ccn5)cc4)nn(c23)[C@@H]2CCC[C@H](C2)NC(=O)CCC1 ; CN1C/C=C/c2cnc(N)c3c(-c4ccc(C(=O)Nc5cc(C(F)(F)F)ccn5)cc4)nn(c23)[C@@H]2CCC[C@H](C2)NC(=O)CCCC1]

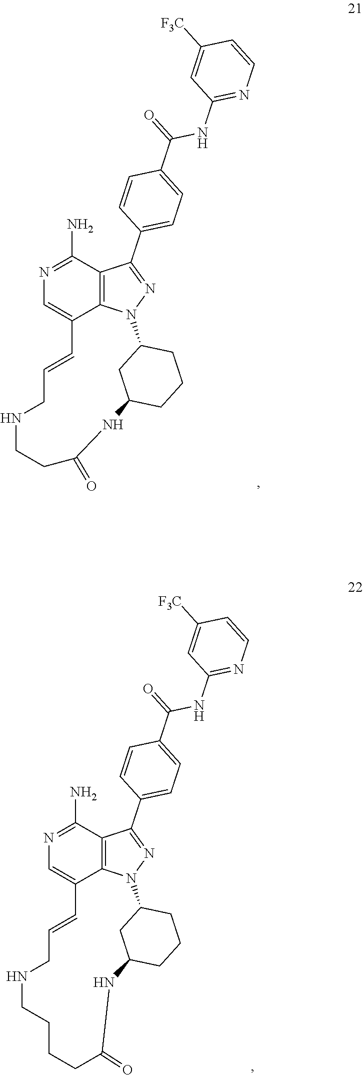 [SMILES: Nc1ncc2c3c1c(-c1ccc(C(=O)Nc4cc(C(F)(F)F)ccn4)cc1)nn3[C@@H]1CCC[C@H](C1)NC(=O)CCNC/C=C/2 ; Nc1ncc2c3c1c(-c1ccc(C(=O)Nc4cc(C(F)(F)F)ccn4)cc1)nn3[C@@H]1CCC[C@H](C1)NC(=O)CCCCNC/C=C/2]

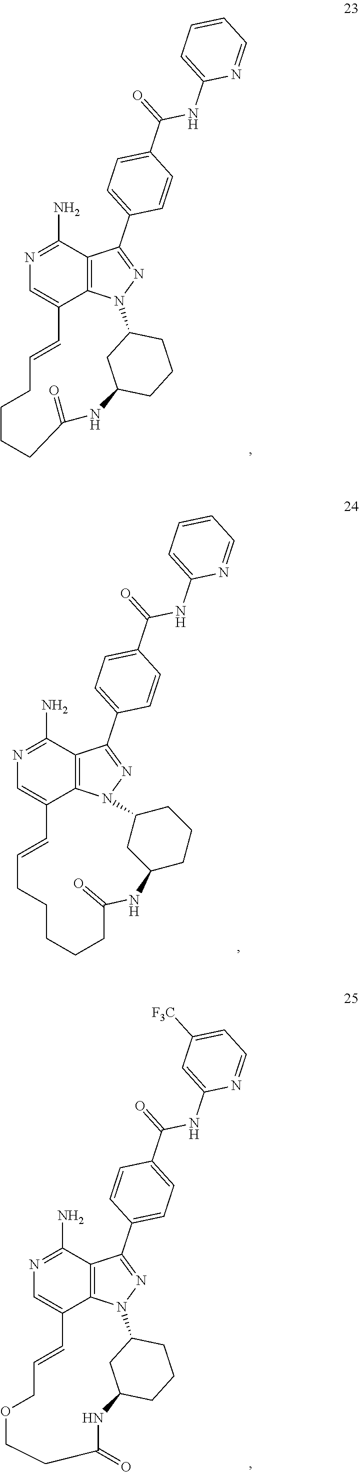 [SMILES: Nc1ncc2c3c1c(-c1ccc(C(=O)Nc4ccccn4)cc1)nn3[C@@H]1CCC[C@H](C1)NC(=O)CCCC/C=C/2 ; Nc1ncc2c3c1c(-c1ccc(C(=O)Nc4ccccn4)cc1)nn3[C@@H]1CCC[C@H](C1)NC(=O)CCCCC/C=C/2 ; Nc1ncc2c3c1c(-c1ccc(C(=O)Nc4cc(C(F)(F)F)ccn4)cc1)nn3[C@@H]1CCC[C@H](C1)NC(=O)CCOC/C=C/2]

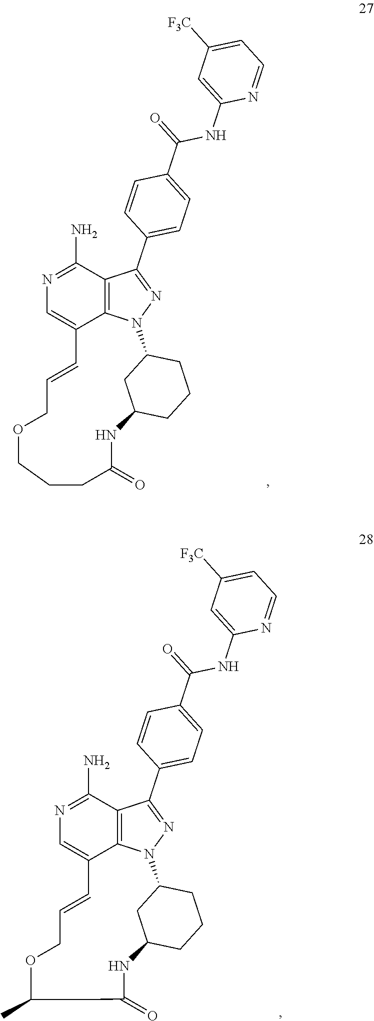 [SMILES: Nc1ncc2c3c1c(-c1ccc(C(=O)Nc4cc(C(F)(F)F)ccn4)cc1)nn3[C@@H]1CCC[C@H](C1)NC(=O)CCCOC/C=C/2 ; C[C@H]1OC/C=C/c2cnc(N)c3c(-c4ccc(C(=O)Nc5cc(C(F)(F)F)ccn5)cc4)nn(c23)[C@@H]2CCC[C@H](C2)NC1=O]

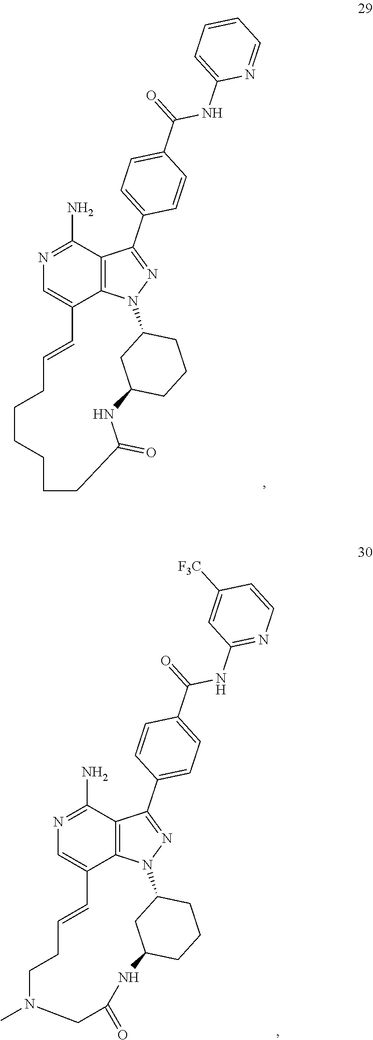 [SMILES: Nc1ncc2c3c1c(-c1ccc(C(=O)Nc4ccccn4)cc1)nn3[C@@H]1CCC[C@H](C1)NC(=O)CCCCCC/C=C/2 ; CN1CC/C=C/c2cnc(N)c3c(-c4ccc(C(=O)Nc5cc(C(F)(F)F)ccn5)cc4)nn(c23)[C@@H]2CCC[C@H](C2)NC(=O)C1]

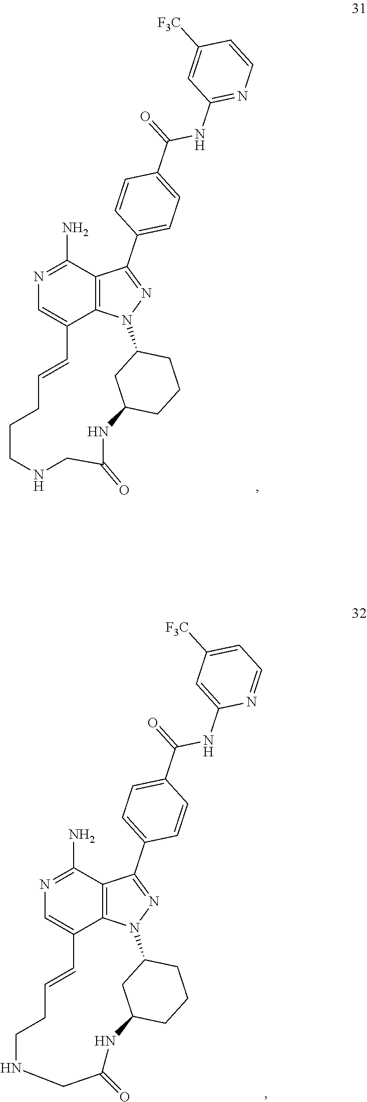 [SMILES: Nc1ncc2c3c1c(-c1ccc(C(=O)Nc4cc(C(F)(F)F)ccn4)cc1)nn3[C@@H]1CCC[C@H](C1)NC(=O)CNCCC/C=C/2 ; Nc1ncc2c3c1c(-c1ccc(C(=O)Nc4cc(C(F)(F)F)ccn4)cc1)nn3[C@@H]1CCC[C@H](C1)NC(=O)CNCC/C=C/2]

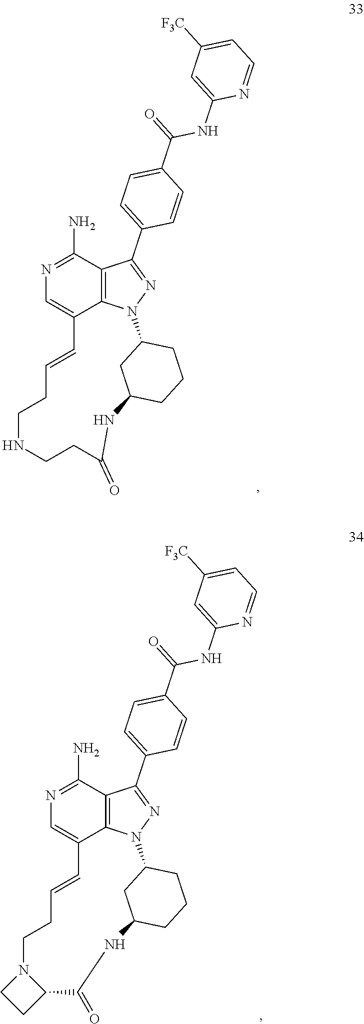 [SMILES: Nc1ncc2c3c1c(-c1ccc(C(=O)Nc4cc(C(F)(F)F)ccn4)cc1)nn3[C@@H]1CCC[C@H](C1)NC(=O)CCNCC/C=C/2 ; Nc1ncc2c3c1c(-c1ccc(C(=O)Nc4cc(C(F)(F)F)ccn4)cc1)nn3[C@@H]1CCC[C@H](C1)NC(=O)[C@@H]1CCN1CC/C=C/2]

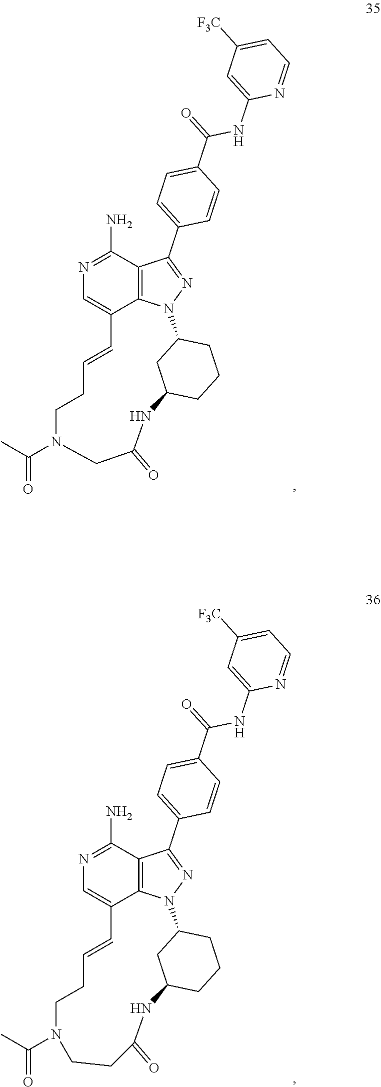 [SMILES: CC(=O)N1CC/C=C/c2cnc(N)c3c(-c4ccc(C(=O)Nc5cc(C(F)(F)F)ccn5)cc4)nn(c23)[C@@H]2CCC[C@H](C2)NC(=O)C1 ; CC(=O)N1CC/C=C/c2cnc(N)c3c(-c4ccc(C(=O)Nc5cc(C(F)(F)F)ccn5)cc4)nn(c23)[C@@H]2CCC[C@H](C2)NC(=O)CC1]

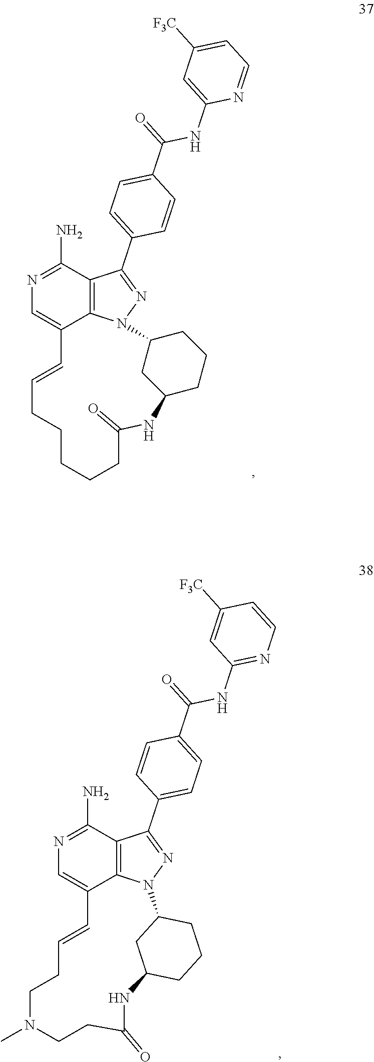 [SMILES: Nc1ncc2c3c1c(-c1ccc(C(=O)Nc4cc(C(F)(F)F)ccn4)cc1)nn3[C@@H]1CCC[C@H](C1)NC(=O)CCCCC/C=C/2 ; CN1CC/C=C/c2cnc(N)c3c(-c4ccc(C(=O)Nc5cc(C(F)(F)F)ccn5)cc4)nn(c23)[C@@H]2CCC[C@H](C2)NC(=O)CC1]

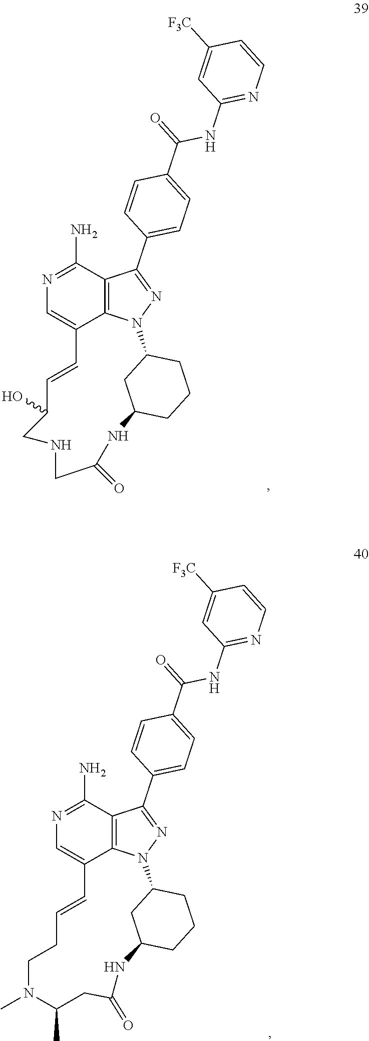 [SMILES: Nc1ncc2c3c1c(-c1ccc(C(=O)Nc4cc(C(F)(F)F)ccn4)cc1)nn3[C@@H]1CCC[C@H](C1)NC(=O)CNCC(O)/C=C/2 ; C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCN1C]

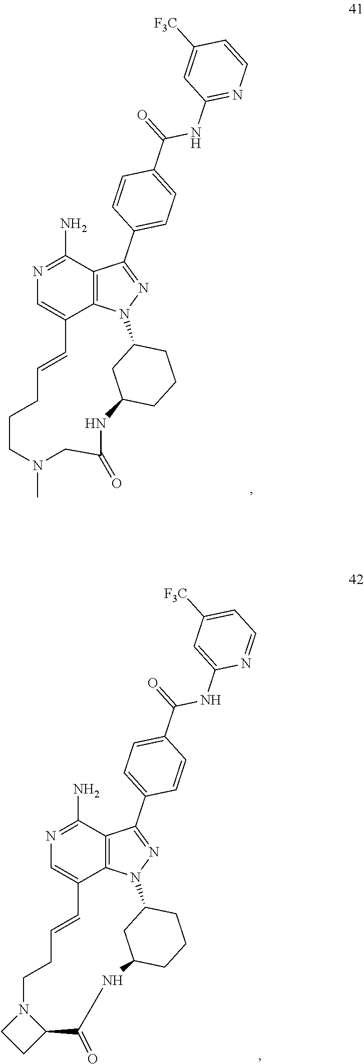 [SMILES: CN1CCC/C=C/c2cnc(N)c3c(-c4ccc(C(=O)Nc5cc(C(F)(F)F)ccn5)cc4)nn(c23)[C@@H]2CCC[C@H](C2)NC(=O)C1 ; Nc1ncc2c3c1c(-c1ccc(C(=O)Nc4cc(C(F)(F)F)ccn4)cc1)nn3[C@@H]1CCC[C@H](C1)NC(=O)[C@H]1CCN1CC/C=C/2]

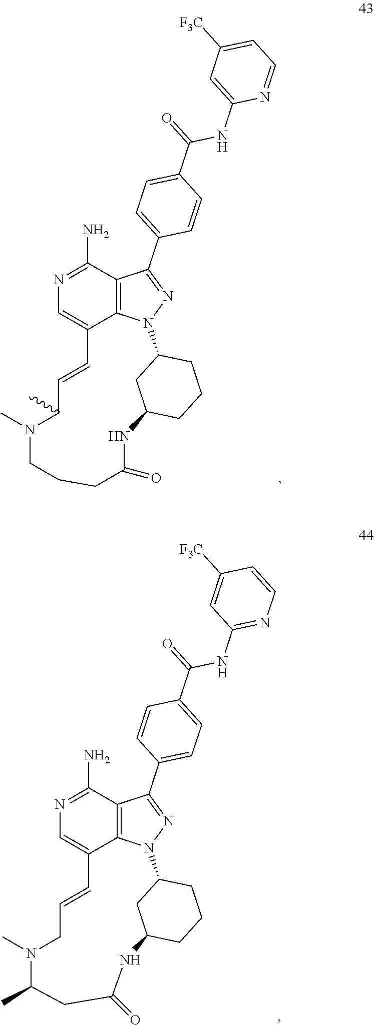 [SMILES: CC1/C=C/c2cnc(N)c3c(-c4ccc(C(=O)Nc5cc(C(F)(F)F)ccn5)cc4)nn(c23)[C@@H]2CCC[C@H](C2)NC(=O)CCCN1C ; C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CN1C]

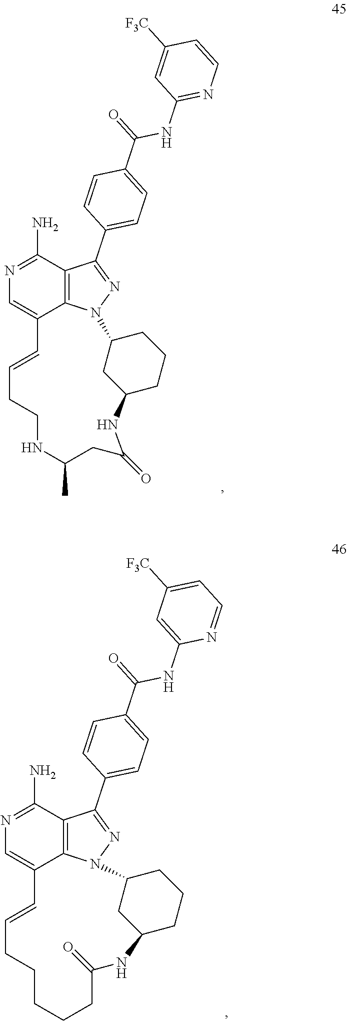 [SMILES: C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCN1 ; Nc1ncc2c3c1c(-c1ccc(C(=O)Nc4cc(C(F)(F)F)ccn4)cc1)nn3[C@@H]1CCC[C@H](C1)NC(=O)CCCCC/C=C/2]

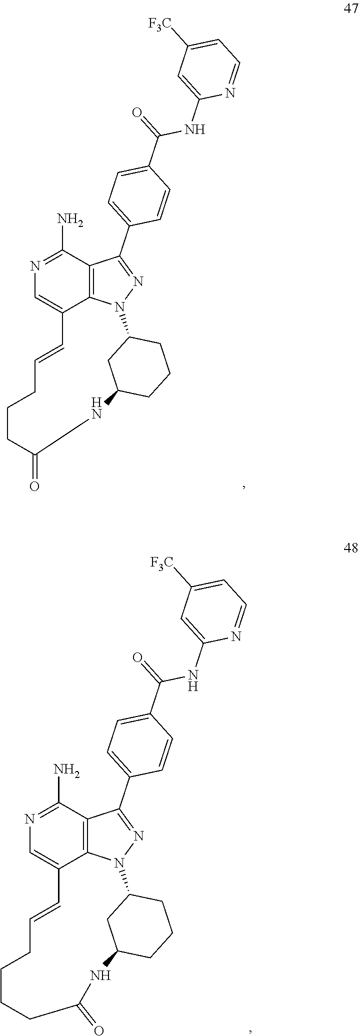 [SMILES: Nc1ncc2c3c1c(-c1ccc(C(=O)Nc4cc(C(F)(F)F)ccn4)cc1)nn3[C@@H]1CCC[C@H](C1)NC(=O)CCC/C=C/2 ; Nc1ncc2c3c1c(-c1ccc(C(=O)Nc4cc(C(F)(F)F)ccn4)cc1)nn3[C@@H]1CCC[C@H](C1)NC(=O)CCCC/C=C/2]

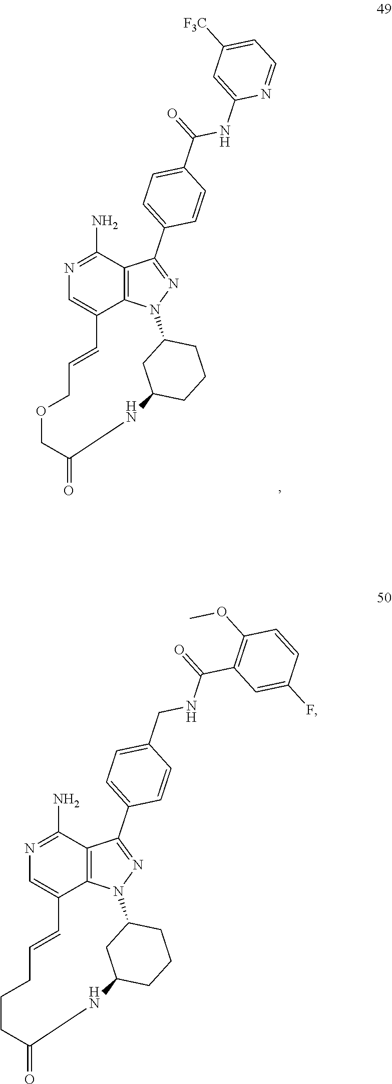 [SMILES: Nc1ncc2c3c1c(-c1ccc(C(=O)Nc4cc(C(F)(F)F)ccn4)cc1)nn3[C@@H]1CCC[C@H](C1)NC(=O)COC/C=C/2 ; COc1ccc(F)cc1C(=O)NCc1ccc(-c2nn3c4c(cnc(N)c24)/C=C/CCCC(=O)N[C@@H]2CCC[C@@H]3C2)cc1]

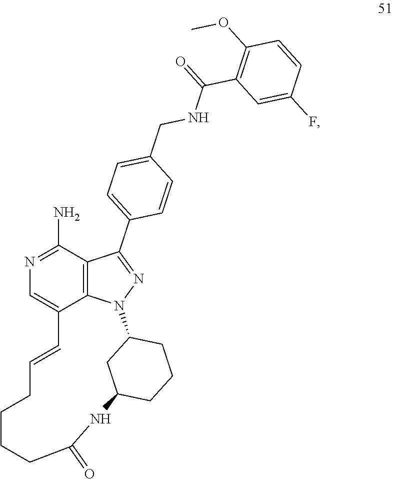 [SMILES: COc1ccc(F)cc1C(=O)NCc1ccc(-c2nn3c4c(cnc(N)c24)/C=C/CCCCC(=O)N[C@@H]2CCC[C@@H]3C2)cc1]

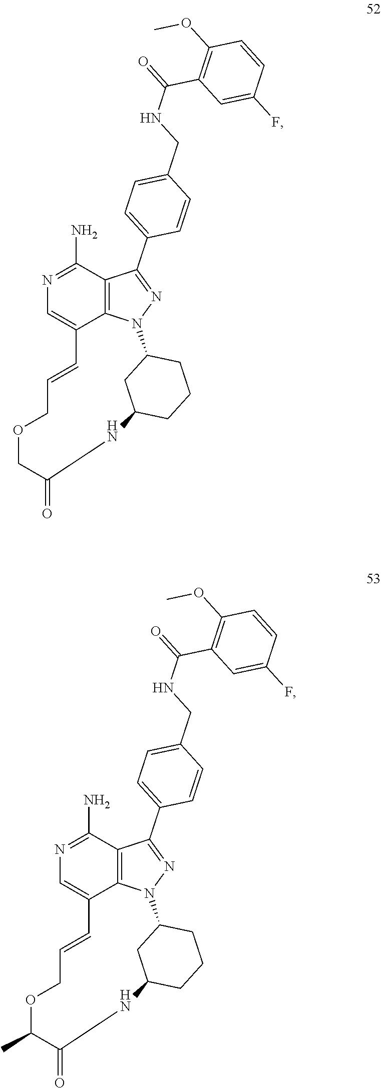 [SMILES: COc1ccc(F)cc1C(=O)NCc1ccc(-c2nn3c4c(cnc(N)c24)/C=C/COCC(=O)N[C@@H]2CCC[C@@H]3C2)cc1 ; COc1ccc(F)cc1C(=O)NCc1ccc(-c2nn3c4c(cnc(N)c24)/C=C/CO[C@H](C)C(=O)N[C@@H]2CCC[C@@H]3C2)cc1]

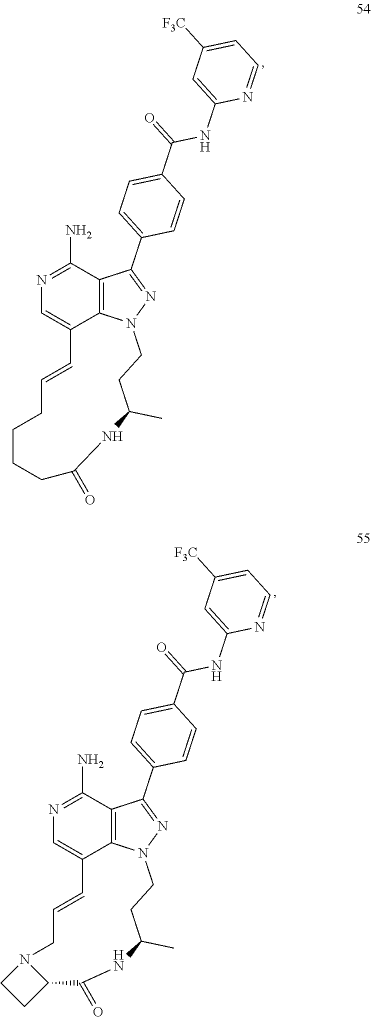 [SMILES: C[C@@H]1CCn2nc(-c3ccc(C(=O)Nc4cc(C(F)(F)F)ccn4)cc3)c3c(N)ncc(c32)/C=C/CCCCC(=O)N1 ; C[C@@H]1CCn2nc(-c3ccc(C(=O)Nc4cc(C(F)(F)F)ccn4)cc3)c3c(N)ncc(c32)/C=C/CN2CC[C@H]2C(=O)N1]

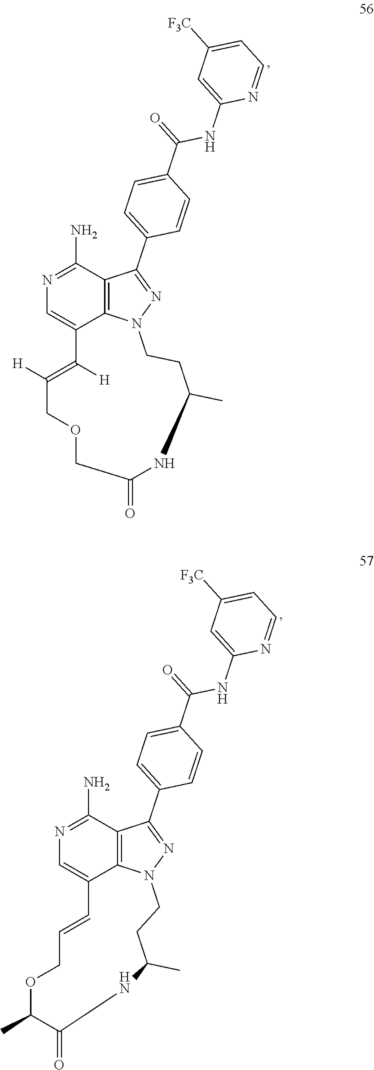 [SMILES: C[C@@H]1CCn2nc(-c3ccc(C(=O)Nc4cc(C(F)(F)F)ccn4)cc3)c3c(N)ncc(c32)/C=C/COCC(=O)N1 ; C[C@@H]1CCn2nc(-c3ccc(C(=O)Nc4cc(C(F)(F)F)ccn4)cc3)c3c(N)ncc(c32)/C=C/CO[C@H](C)C(=O)N1]

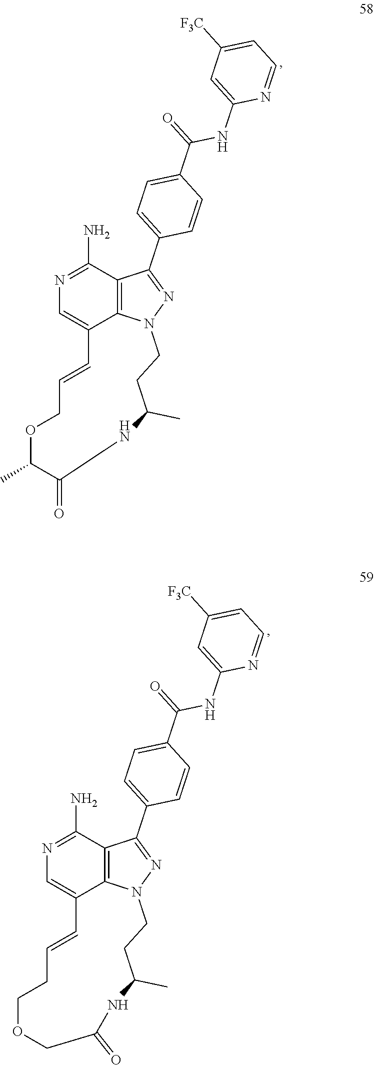 [SMILES: C[C@@H]1CCn2nc(-c3ccc(C(=O)Nc4cc(C(F)(F)F)ccn4)cc3)c3c(N)ncc(c32)/C=C/CO[C@@H](C)C(=O)N1 ; C[C@@H]1CCn2nc(-c3ccc(C(=O)Nc4cc(C(F)(F)F)ccn4)cc3)c3c(N)ncc(c32)/C=C/CCOCC(=O)N1]

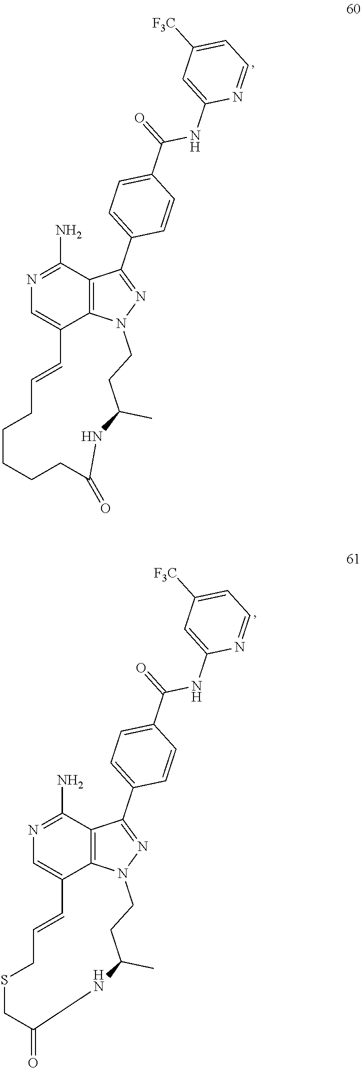 [SMILES: C[C@@H]1CCn2nc(-c3ccc(C(=O)Nc4cc(C(F)(F)F)ccn4)cc3)c3c(N)ncc(c32)/C=C/CCCCCC(=O)N1 ; C[C@@H]1CCn2nc(-c3ccc(C(=O)Nc4cc(C(F)(F)F)ccn4)cc3)c3c(N)ncc(c32)/C=C/CSCC(=O)N1]

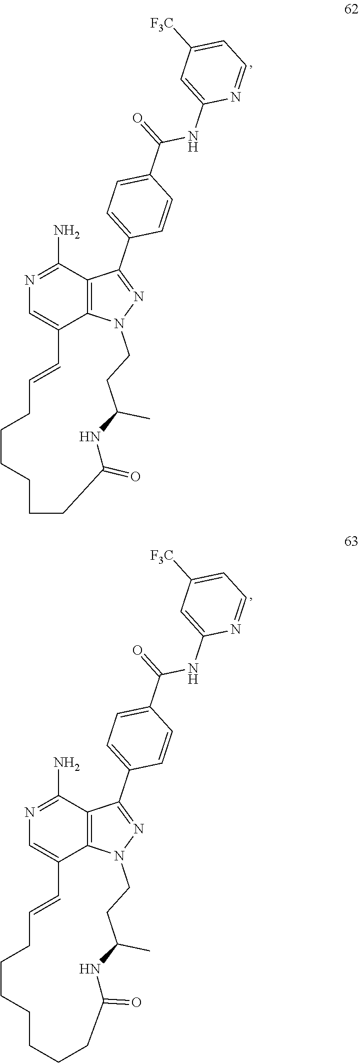 [SMILES: C[C@@H]1CCn2nc(-c3ccc(C(=O)Nc4cc(C(F)(F)F)ccn4)cc3)c3c(N)ncc(c32)/C=C/CCCCCCC(=O)N1 ; C[C@@H]1CCn2nc(-c3ccc(C(=O)Nc4cc(C(F)(F)F)ccn4)cc3)c3c(N)ncc(c32)/C=C/CCCCCCCC(=O)N1]

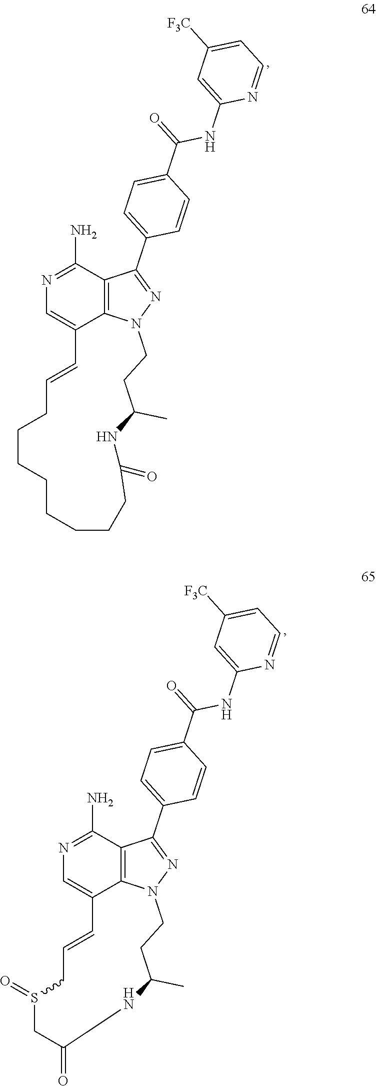 [SMILES: C[C@@H]1CCn2nc(-c3ccc(C(=O)Nc4cc(C(F)(F)F)ccn4)cc3)c3c(N)ncc(c32)/C=C/CCCCCCCCC(=O)N1 ; C[C@@H]1CCn2nc(-c3ccc(C(=O)Nc4cc(C(F)(F)F)ccn4)cc3)c3c(N)ncc(c32)/C=C/CS(=O)CC(=O)N1]

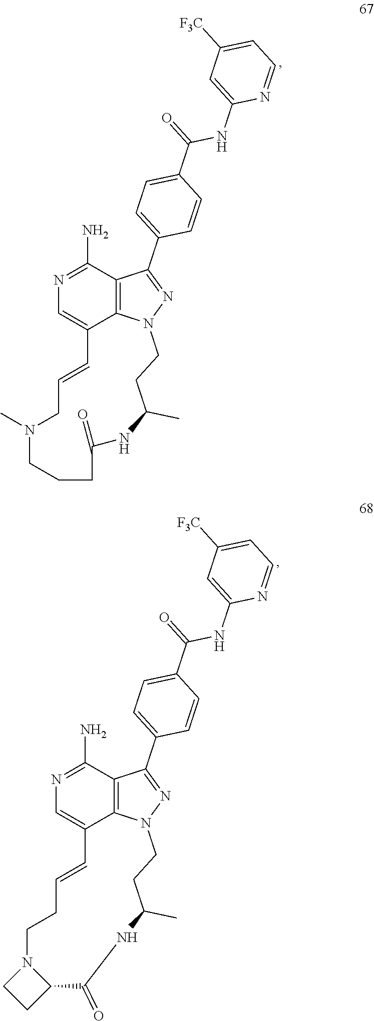 [SMILES: C[C@@H]1CCn2nc(-c3ccc(C(=O)Nc4cc(C(F)(F)F)ccn4)cc3)c3c(N)ncc(c32)/C=C/CN(C)CCCC(=O)N1 ; C[C@@H]1CCn2nc(-c3ccc(C(=O)Nc4cc(C(F)(F)F)ccn4)cc3)c3c(N)ncc(c32)/C=C/CCN2CC[C@H]2C(=O)N1]

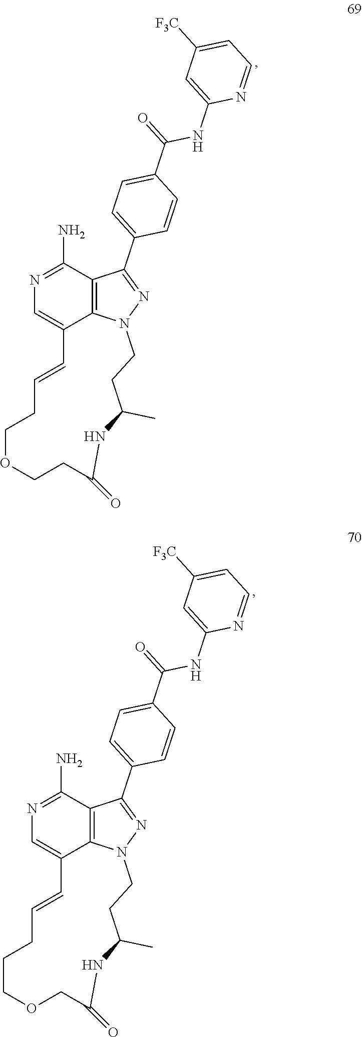 [SMILES: C[C@@H]1CCn2nc(-c3ccc(C(=O)Nc4cc(C(F)(F)F)ccn4)cc3)c3c(N)ncc(c32)/C=C/CCOCCC(=O)N1 ; C[C@@H]1CCn2nc(-c3ccc(C(=O)Nc4cc(C(F)(F)F)ccn4)cc3)c3c(N)ncc(c32)/C=C/CCCOCC(=O)N1]

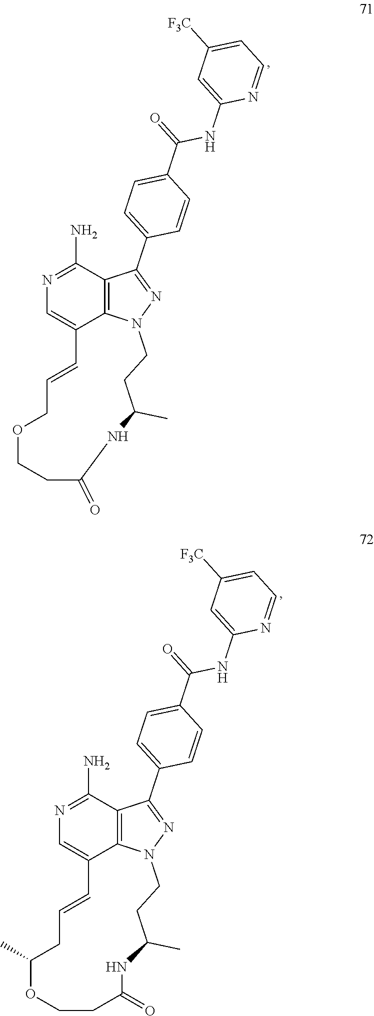 [SMILES: C[C@@H]1CCn2nc(-c3ccc(C(=O)Nc4cc(C(F)(F)F)ccn4)cc3)c3c(N)ncc(c32)/C=C/COCCC(=O)N1 ; C[C@@H]1CCn2nc(-c3ccc(C(=O)Nc4cc(C(F)(F)F)ccn4)cc3)c3c(N)ncc(c32)/C=C/C[C@@H](C)OCCC(=O)N1]

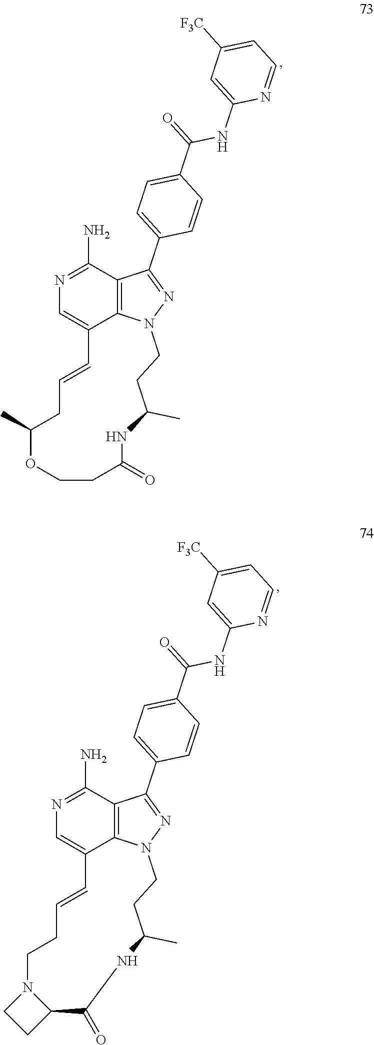 [SMILES: C[C@@H]1CCn2nc(-c3ccc(C(=O)Nc4cc(C(F)(F)F)ccn4)cc3)c3c(N)ncc(c32)/C=C/C[C@H](C)OCCC(=O)N1 ; C[C@@H]1CCn2nc(-c3ccc(C(=O)Nc4cc(C(F)(F)F)ccn4)cc3)c3c(N)ncc(c32)/C=C/CCN2CC[C@@H]2C(=O)N1]

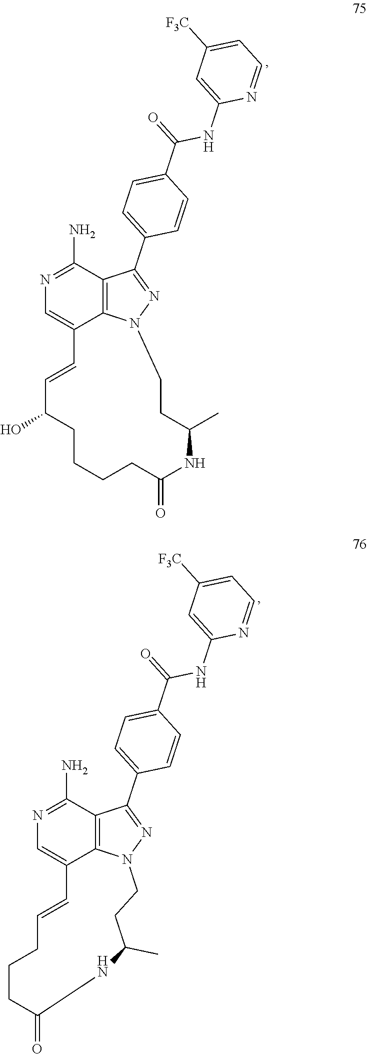 [SMILES: C[C@@H]1CCn2nc(-c3ccc(C(=O)Nc4cc(C(F)(F)F)ccn4)cc3)c3c(N)ncc(c32)/C=C/[C@@H](O)CCCCC(=O)N1 ; C[C@@H]1CCn2nc(-c3ccc(C(=O)Nc4cc(C(F)(F)F)ccn4)cc3)c3c(N)ncc(c32)/C=C/CCCC(=O)N1]

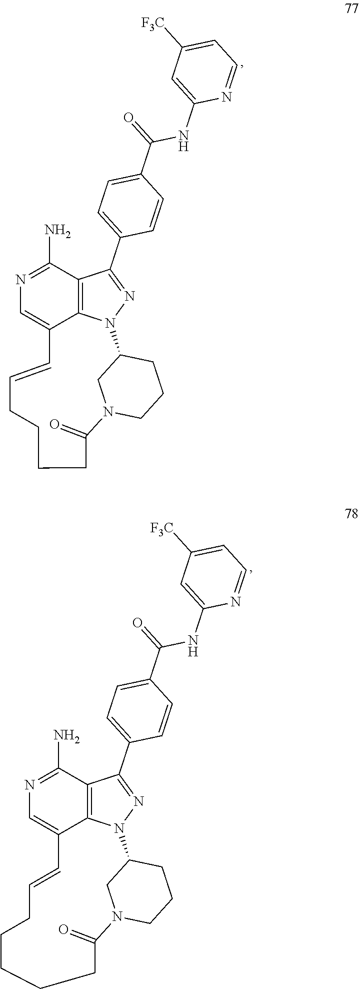 [SMILES: Nc1ncc2c3c1c(-c1ccc(C(=O)Nc4cc(C(F)(F)F)ccn4)cc1)nn3[C@@H]1CCCN(C1)C(=O)CCCC/C=C/2 ; Nc1ncc2c3c1c(-c1ccc(C(=O)Nc4cc(C(F)(F)F)ccn4)cc1)nn3[C@@H]1CCCN(C1)C(=O)CCCCC/C=C/2]

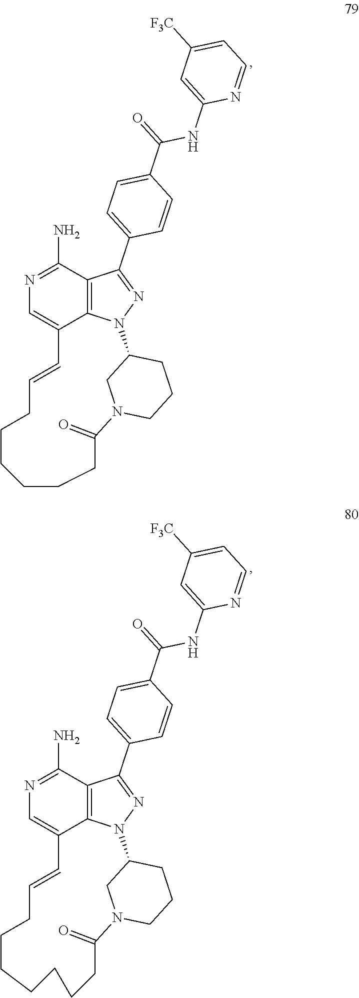 [SMILES: Nc1ncc2c3c1c(-c1ccc(C(=O)Nc4cc(C(F)(F)F)ccn4)cc1)nn3[C@@H]1CCCN(C1)C(=O)CCCCCC/C=C/2 ; Nc1ncc2c3c1c(-c1ccc(C(=O)Nc4cc(C(F)(F)F)ccn4)cc1)nn3[C@@H]1CCCN(C1)C(=O)CCCCCCC/C=C/2]

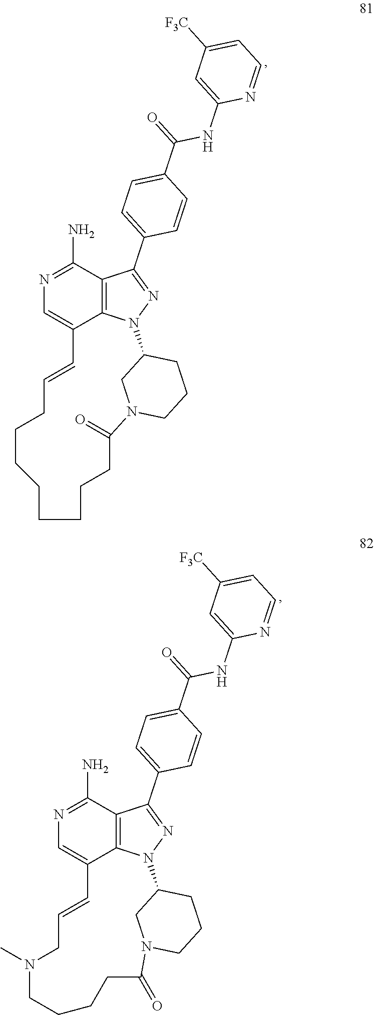 [SMILES: Nc1ncc2c3c1c(-c1ccc(C(=O)Nc4cc(C(F)(F)F)ccn4)cc1)nn3[C@@H]1CCCN(C1)C(=O)CCCCCCCC/C=C/2 ; CN1C/C=C/c2cnc(N)c3c(-c4ccc(C(=O)Nc5cc(C(F)(F)F)ccn5)cc4)nn(c23)[C@@H]2CCCN(C2)C(=O)CCCC1]

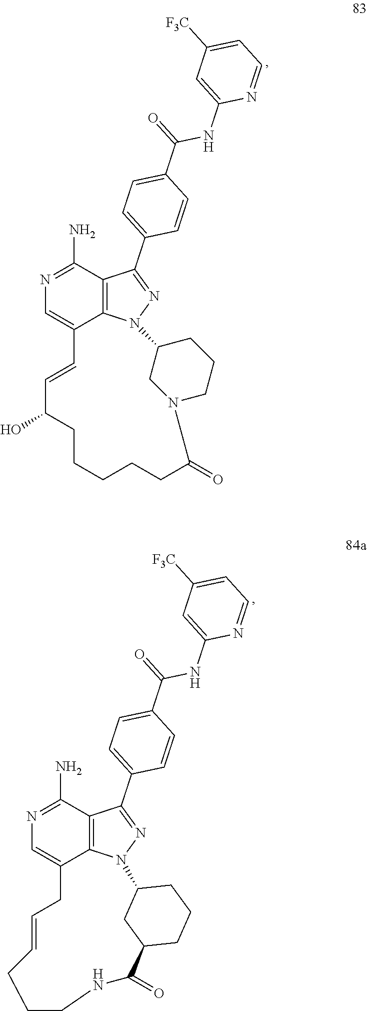 [SMILES: Nc1ncc2c3c1c(-c1ccc(C(=O)Nc4cc(C(F)(F)F)ccn4)cc1)nn3[C@@H]1CCCN(C1)C(=O)CCCCC[C@H](O)/C=C/2 ; Nc1ncc2c3c1c(-c1ccc(C(=O)Nc4cc(C(F)(F)F)ccn4)cc1)nn3[C@@H]1CCC[C@H](C1)C(=O)NCCC/C=C/C2]

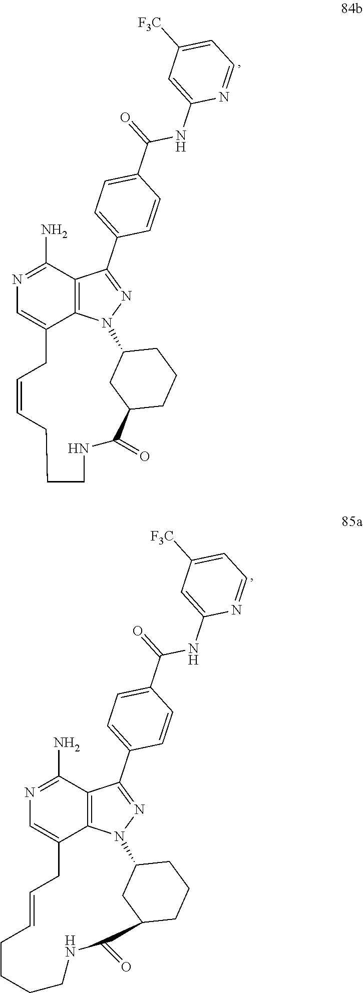 [SMILES: Nc1ncc2c3c1c(-c1ccc(C(=O)Nc4cc(C(F)(F)F)ccn4)cc1)nn3[C@@H]1CCC[C@H](C1)C(=O)NCCC/C=C\C2 ; Nc1ncc2c3c1c(-c1ccc(C(=O)Nc4cc(C(F)(F)F)ccn4)cc1)nn3[C@@H]1CCC[C@H](C1)C(=O)NCCCC/C=C/C2]

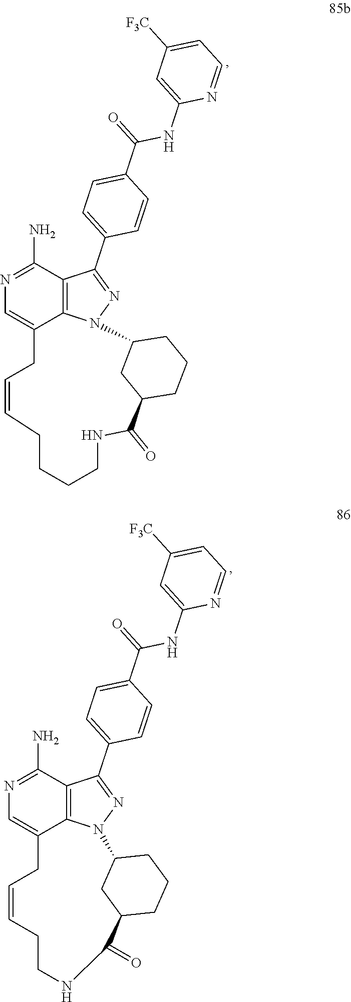 [SMILES: Nc1ncc2c3c1c(-c1ccc(C(=O)Nc4cc(C(F)(F)F)ccn4)cc1)nn3[C@@H]1CCC[C@H](C1)C(=O)NCCCC/C=C\C2 ; Nc1ncc2c3c1c(-c1ccc(C(=O)Nc4cc(C(F)(F)F)ccn4)cc1)nn3[C@@H]1CCC[C@H](C1)C(=O)NCC/C=C\C2]

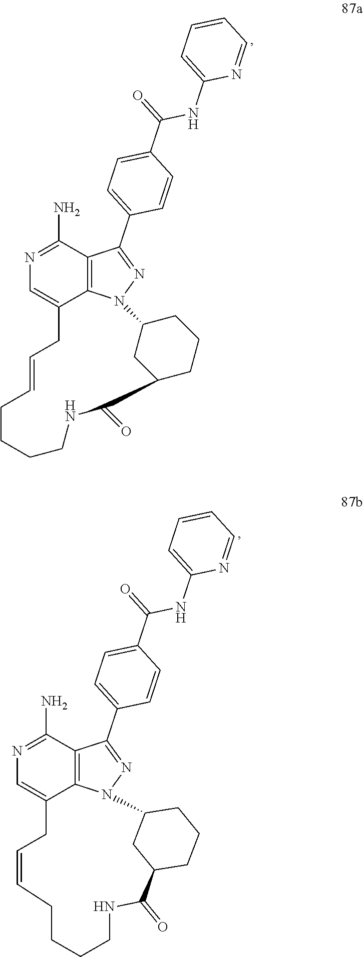 [SMILES: Nc1ncc2c3c1c(-c1ccc(C(=O)Nc4ccccn4)cc1)nn3[C@@H]1CCC[C@H](C1)C(=O)NCCCC/C=C/C2 ; Nc1ncc2c3c1c(-c1ccc(C(=O)Nc4ccccn4)cc1)nn3[C@@H]1CCC[C@H](C1)C(=O)NCCCC/C=C\C2]

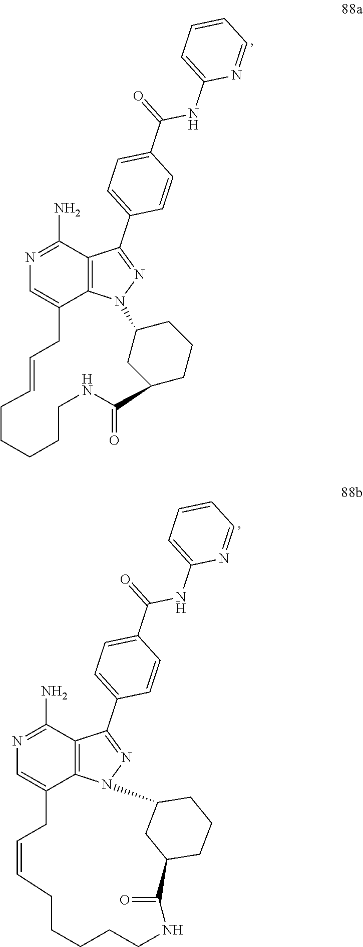 [SMILES: Nc1ncc2c3c1c(-c1ccc(C(=O)Nc4ccccn4)cc1)nn3[C@@H]1CCC[C@H](C1)C(=O)NCCCCC/C=C/C2 ; Nc1ncc2c3c1c(-c1ccc(C(=O)Nc4ccccn4)cc1)nn3[C@@H]1CCC[C@H](C1)C(=O)NCCCCC/C=C\C2]

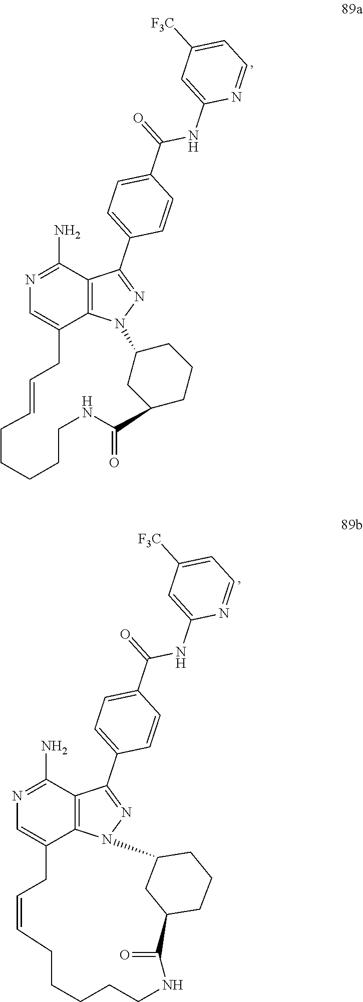 [SMILES: Nc1ncc2c3c1c(-c1ccc(C(=O)Nc4cc(C(F)(F)F)ccn4)cc1)nn3[C@@H]1CCC[C@H](C1)C(=O)NCCCCC/C=C/C2 ; Nc1ncc2c3c1c(-c1ccc(C(=O)Nc4cc(C(F)(F)F)ccn4)cc1)nn3[C@@H]1CCC[C@H](C1)C(=O)NCCCCC/C=C\C2]

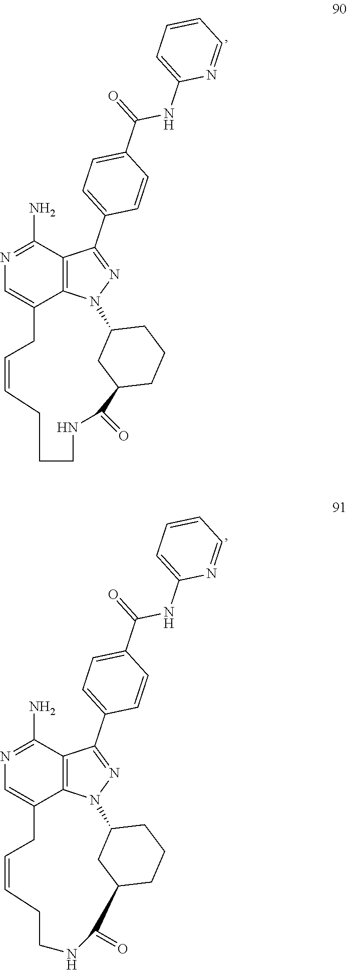 [SMILES: Nc1ncc2c3c1c(-c1ccc(C(=O)Nc4ccccn4)cc1)nn3[C@@H]1CCC[C@H](C1)C(=O)NCCC/C=C\C2 ; Nc1ncc2c3c1c(-c1ccc(C(=O)Nc4ccccn4)cc1)nn3[C@@H]1CCC[C@H](C1)C(=O)NCC/C=C\C2]

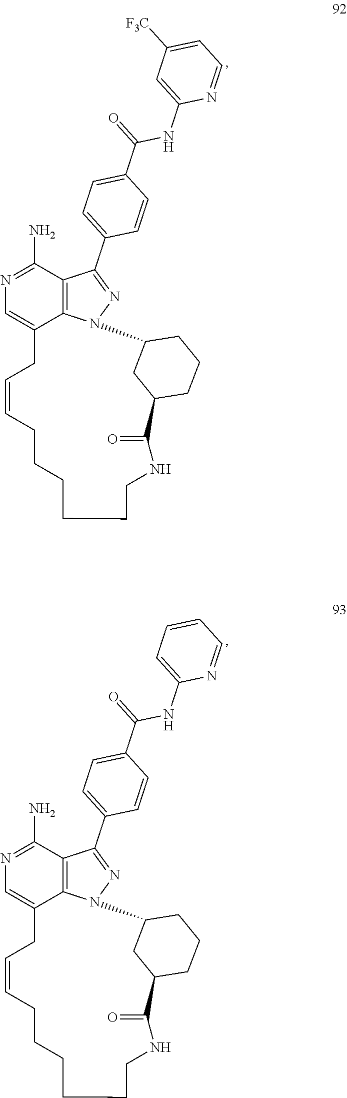 [SMILES: Nc1ncc2c3c1c(-c1ccc(C(=O)Nc4cc(C(F)(F)F)ccn4)cc1)nn3[C@@H]1CCC[C@H](C1)C(=O)NCCCCCC/C=C\C2 ; Nc1ncc2c3c1c(-c1ccc(C(=O)Nc4ccccn4)cc1)nn3[C@@H]1CCC[C@H](C1)C(=O)NCCCCCC/C=C\C2]

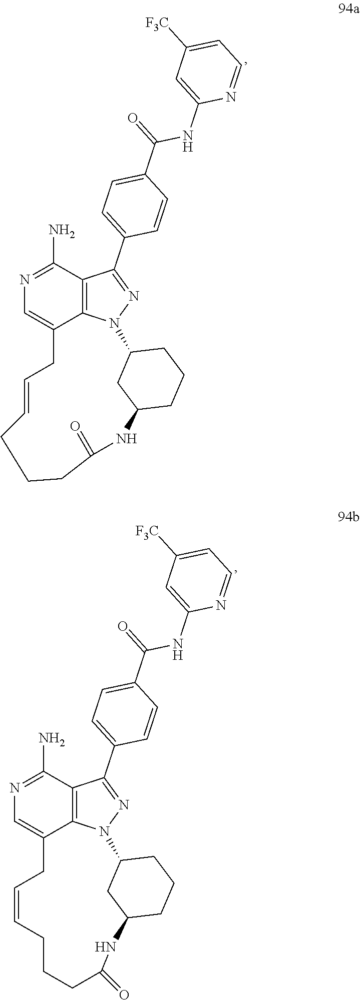 [SMILES: Nc1ncc2c3c1c(-c1ccc(C(=O)Nc4cc(C(F)(F)F)ccn4)cc1)nn3[C@@H]1CCC[C@H](C1)NC(=O)CCC/C=C/C2 ; Nc1ncc2c3c1c(-c1ccc(C(=O)Nc4cc(C(F)(F)F)ccn4)cc1)nn3[C@@H]1CCC[C@H](C1)NC(=O)CCC/C=C\C2]

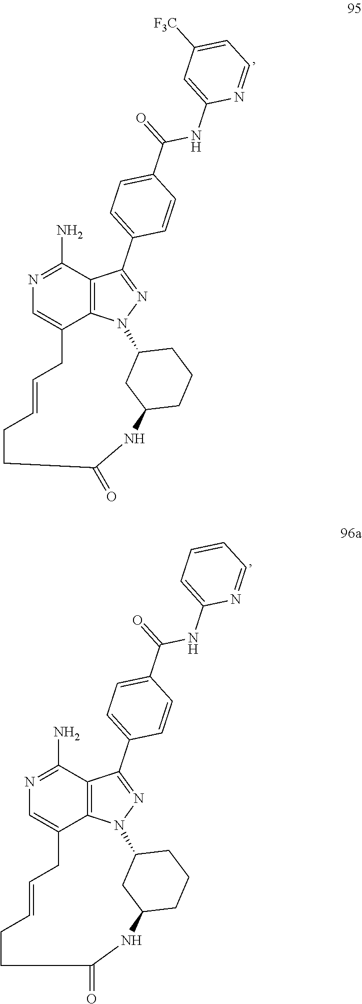 [SMILES: Nc1ncc2c3c1c(-c1ccc(C(=O)Nc4cc(C(F)(F)F)ccn4)cc1)nn3[C@@H]1CCC[C@H](C1)NC(=O)CC/C=C/C2 ; Nc1ncc2c3c1c(-c1ccc(C(=O)Nc4ccccn4)cc1)nn3[C@@H]1CCC[C@H](C1)NC(=O)CC/C=C/C2]

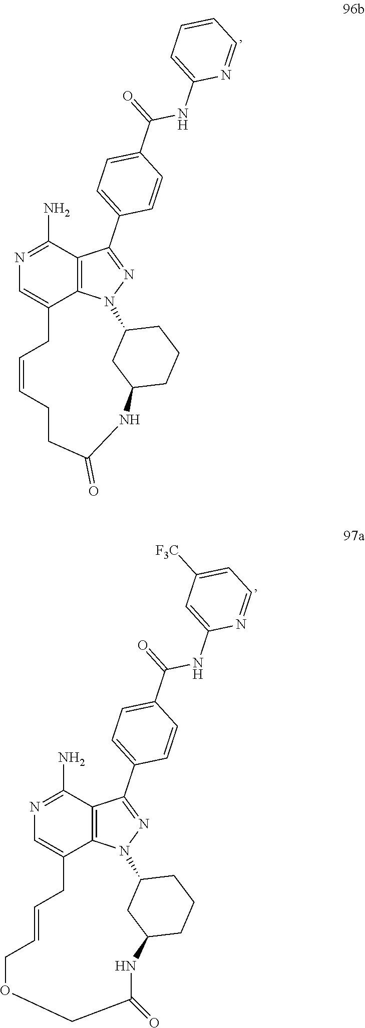 [SMILES: Nc1ncc2c3c1c(-c1ccc(C(=O)Nc4ccccn4)cc1)nn3[C@@H]1CCC[C@H](C1)NC(=O)CC/C=C\C2 ; Nc1ncc2c3c1c(-c1ccc(C(=O)Nc4cc(C(F)(F)F)ccn4)cc1)nn3[C@@H]1CCC[C@H](C1)NC(=O)COC/C=C/C2]

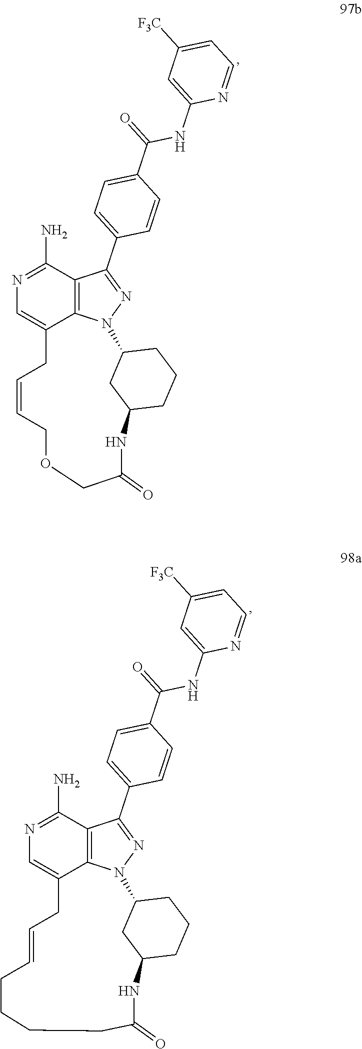 [SMILES: Nc1ncc2c3c1c(-c1ccc(C(=O)Nc4cc(C(F)(F)F)ccn4)cc1)nn3[C@@H]1CCC[C@H](C1)NC(=O)COC/C=C\C2 ; Nc1ncc2c3c1c(-c1ccc(C(=O)Nc4cc(C(F)(F)F)ccn4)cc1)nn3[C@@H]1CCC[C@H](C1)NC(=O)CCCC/C=C/C2]

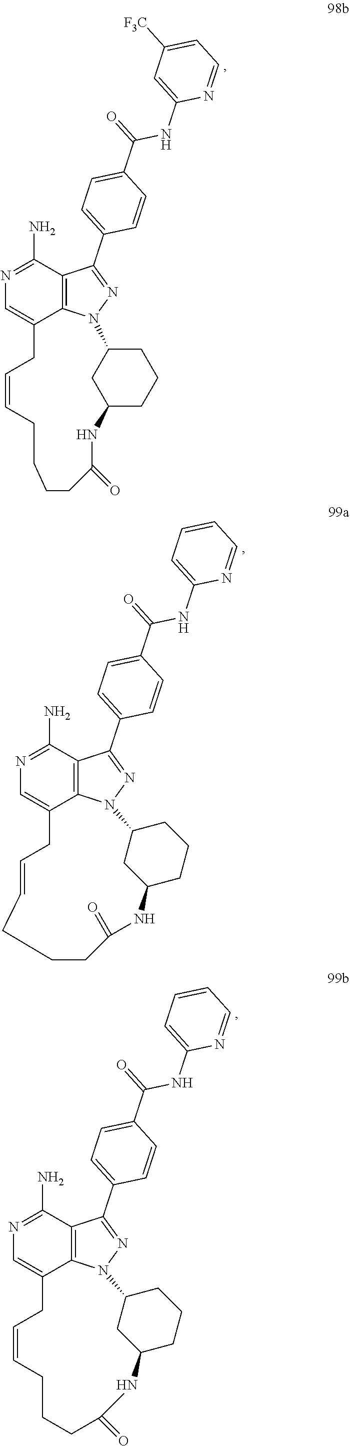 [SMILES: Nc1ncc2c3c1c(-c1ccc(C(=O)Nc4cc(C(F)(F)F)ccn4)cc1)nn3[C@@H]1CCC[C@H](C1)NC(=O)CCCC/C=C\C2 ; Nc1ncc2c3c1c(-c1ccc(C(=O)Nc4ccccn4)cc1)nn3[C@@H]1CCC[C@H](C1)NC(=O)CCC/C=C/C2 ; Nc1ncc2c3c1c(-c1ccc(C(=O)Nc4ccccn4)cc1)nn3[C@@H]1CCC[C@H](C1)NC(=O)CCC/C=C\C2]

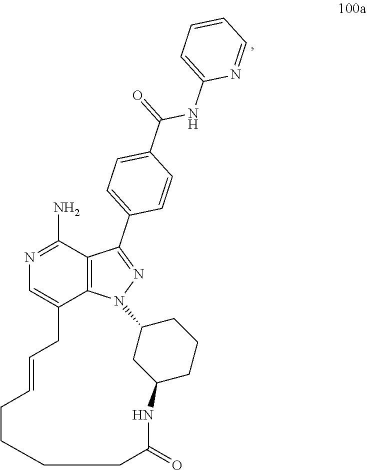 [SMILES: Nc1ncc2c3c1c(-c1ccc(C(=O)Nc4ccccn4)cc1)nn3[C@@H]1CCC[C@H](C1)NC(=O)CCCC/C=C/C2]

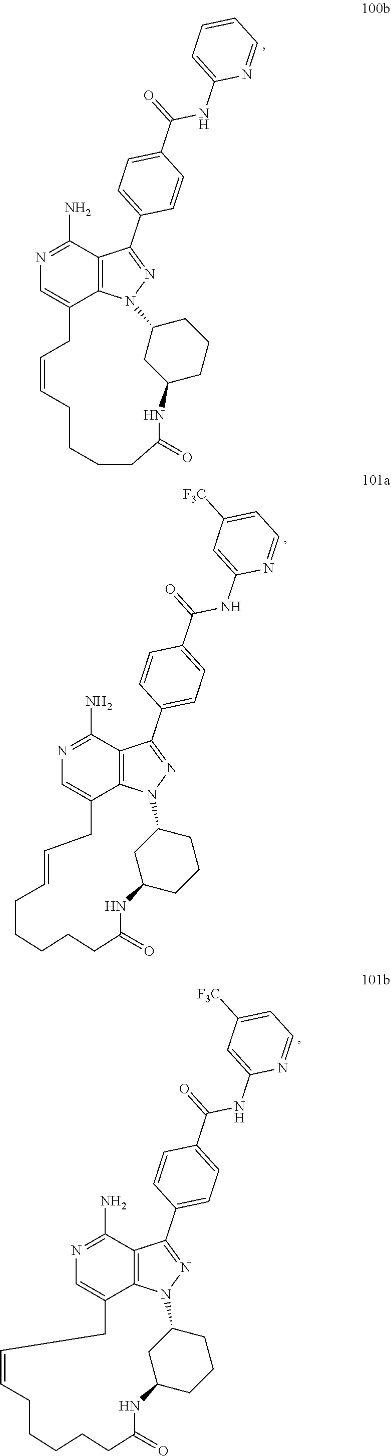

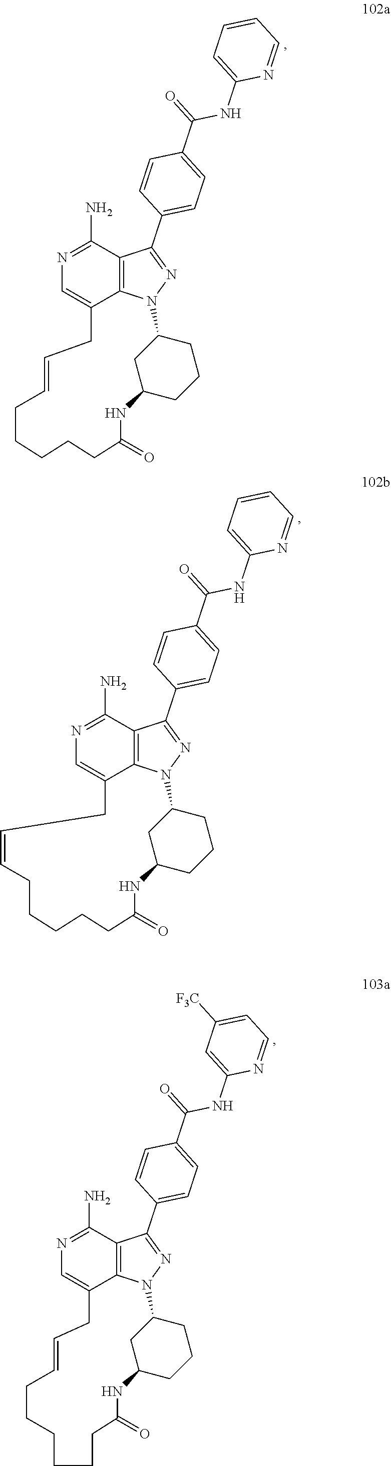 [SMILES: Nc1ncc2c3c1c(-c1ccc(C(=O)Nc4ccccn4)cc1)nn3[C@@H]1CCC[C@H](C1)NC(=O)CCCCC/C=C/C2 ; Nc1ncc2c3c1c(-c1ccc(C(=O)Nc4ccccn4)cc1)nn3[C@@H]1CCC[C@H](C1)NC(=O)CCCCC/C=C\C2 ; Nc1ncc2c3c1c(-c1ccc(C(=O)Nc4cc(C(F)(F)F)ccn4)cc1)nn3[C@@H]1CCC[C@H](C1)NC(=O)CCCCCC/C=C/C2]

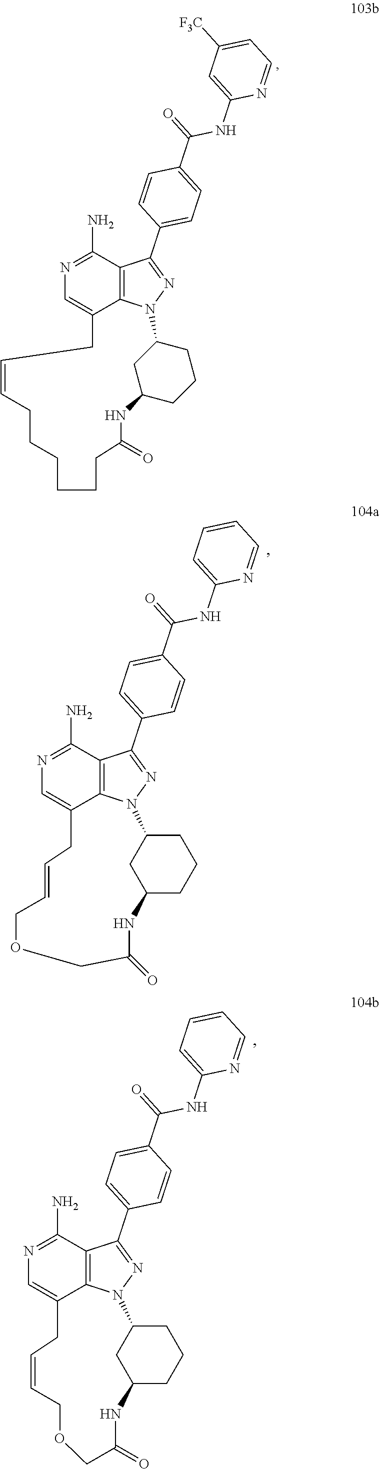 [SMILES: Nc1ncc2c3c1c(-c1ccc(C(=O)Nc4cc(C(F)(F)F)ccn4)cc1)nn3[C@@H]1CCC[C@H](C1)NC(=O)CCCCCC/C=C\C2 ; Nc1ncc2c3c1c(-c1ccc(C(=O)Nc4ccccn4)cc1)nn3[C@@H]1CCC[C@H](C1)NC(=O)COC/C=C/C2 ; Nc1ncc2c3c1c(-c1ccc(C(=O)Nc4ccccn4)cc1)nn3[C@@H]1CCC[C@H](C1)NC(=O)COC/C=C\C2]

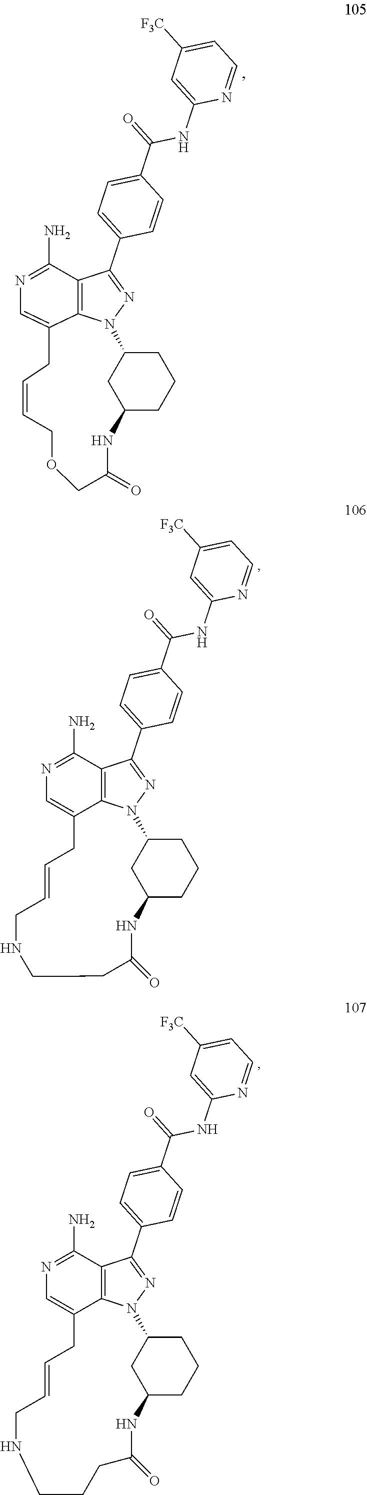

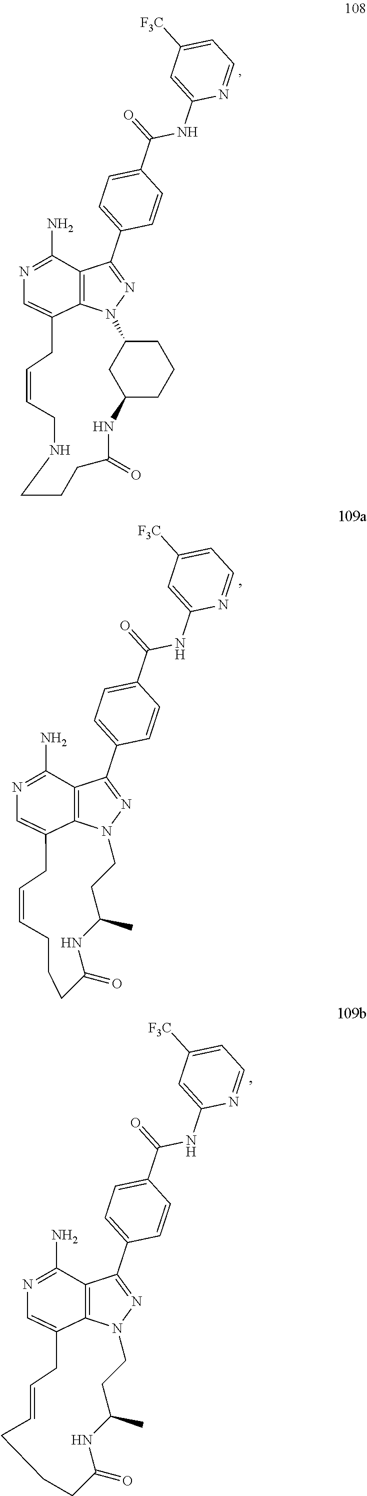 [SMILES: Nc1ncc2c3c1c(-c1ccc(C(=O)Nc4cc(C(F)(F)F)ccn4)cc1)nn3[C@@H]1CCC[C@H](C1)NC(=O)CCCNC/C=C\C2 ; C[C@@H]1CCn2nc(-c3ccc(C(=O)Nc4cc(C(F)(F)F)ccn4)cc3)c3c(N)ncc(c32)C/C=C\CCCC(=O)N1 ; C[C@@H]1CCn2nc(-c3ccc(C(=O)Nc4cc(C(F)(F)F)ccn4)cc3)c3c(N)ncc(c32)C/C=C/CCCC(=O)N1]

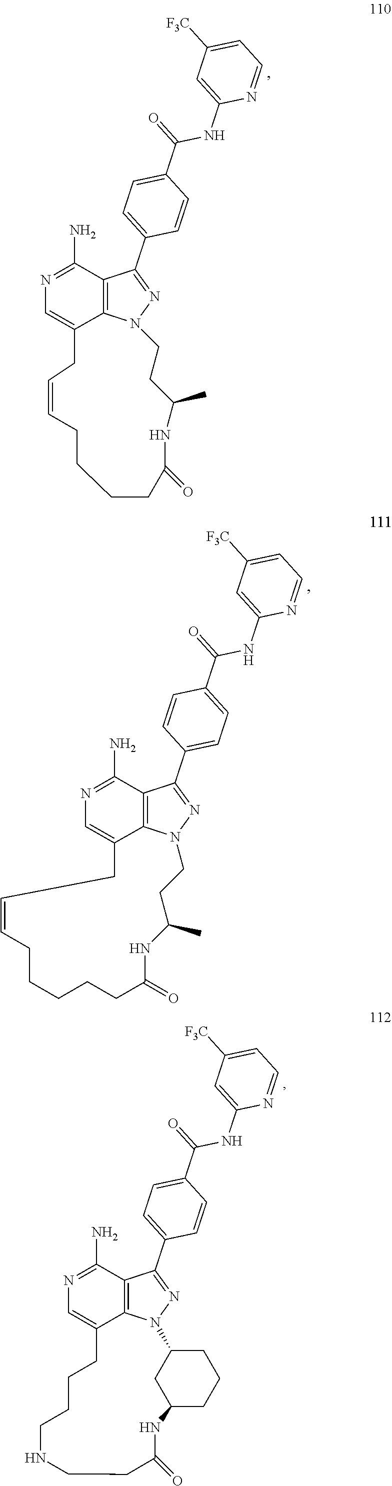

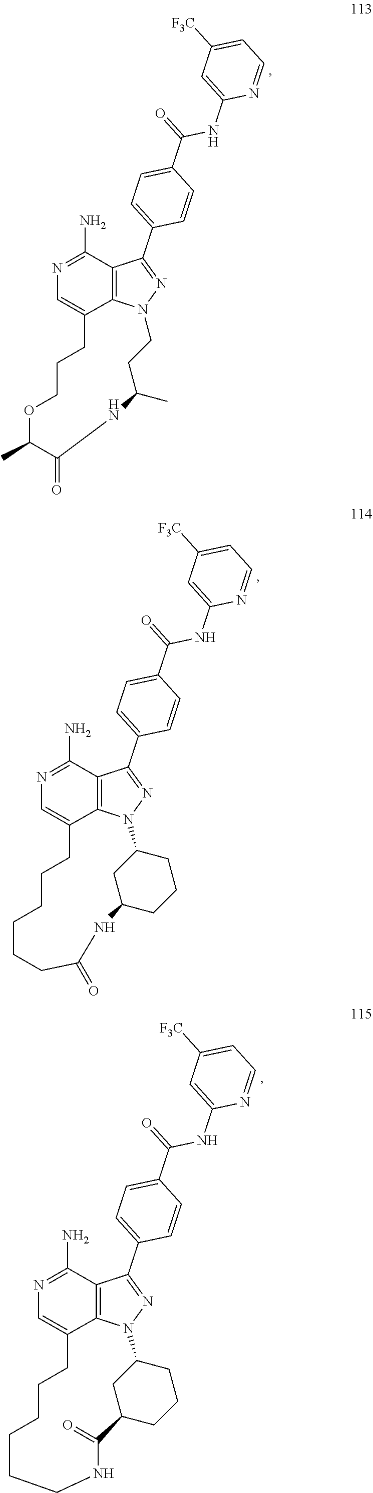 [SMILES: C[C@@H]1CCn2nc(-c3ccc(C(=O)Nc4cc(C(F)(F)F)ccn4)cc3)c3c(N)ncc(c32)CCCO[C@H](C)C(=O)N1 ; Nc1ncc2c3c1c(-c1ccc(C(=O)Nc4cc(C(F)(F)F)ccn4)cc1)nn3[C@@H]1CCC[C@H](C1)NC(=O)CCCCCC2 ; Nc1ncc2c3c1c(-c1ccc(C(=O)Nc4cc(C(F)(F)F)ccn4)cc1)nn3[C@@H]1CCC[C@H](C1)C(=O)NCCCCCC2]

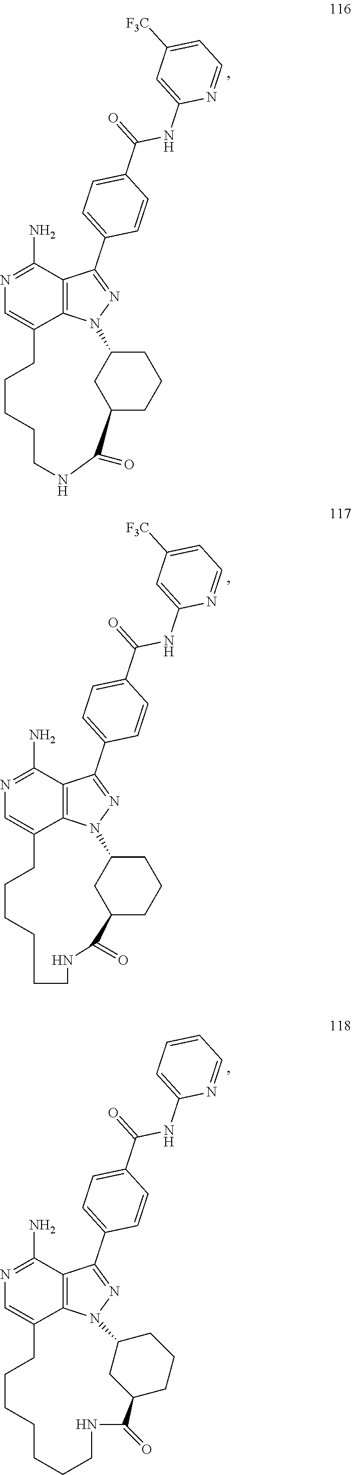 [SMILES: Nc1ncc2c3c1c(-c1ccc(C(=O)Nc4cc(C(F)(F)F)ccn4)cc1)nn3[C@@H]1CCC[C@H](C1)C(=O)NCCCCC2 ; Nc1ncc2c3c1c(-c1ccc(C(=O)Nc4cc(C(F)(F)F)ccn4)cc1)nn3[C@@H]1CCC[C@H](C1)C(=O)NCCCCCC2 ; Nc1ncc2c3c1c(-c1ccc(C(=O)Nc4ccccn4)cc1)nn3[C@@H]1CCC[C@H](C1)C(=O)NCCCCCCC2]

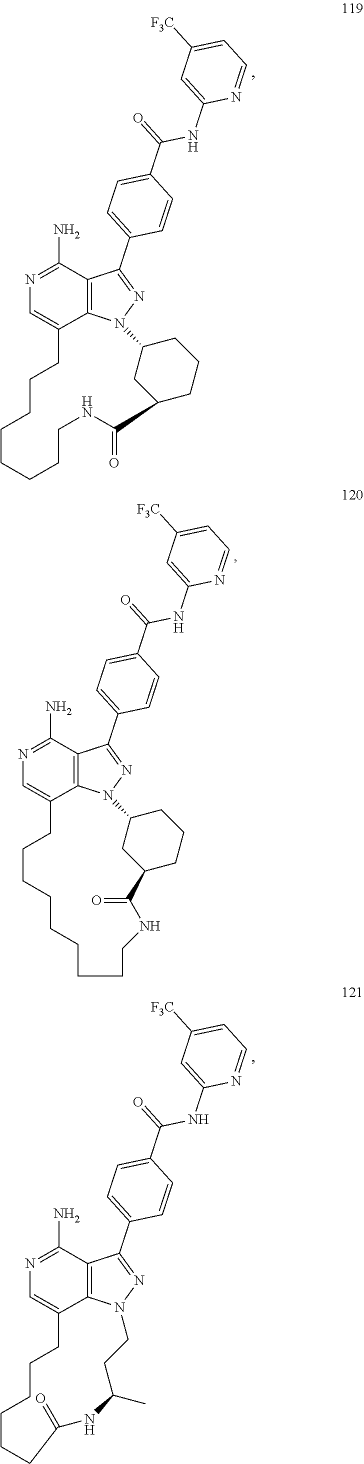 [SMILES: Nc1ncc2c3c1c(-c1ccc(C(=O)Nc4cc(C(F)(F)F)ccn4)cc1)nn3[C@@H]1CCC[C@H](C1)C(=O)NCCCCCCCC2 ; Nc1ncc2c3c1c(-c1ccc(C(=O)Nc4cc(C(F)(F)F)ccn4)cc1)nn3[C@@H]1CCC[C@H](C1)C(=O)NCCCCCCCCC2 ; C[C@@H]1CCn2nc(-c3ccc(C(=O)Nc4cc(C(F)(F)F)ccn4)cc3)c3c(N)ncc(c32)CCCCCCC(=O)N1]

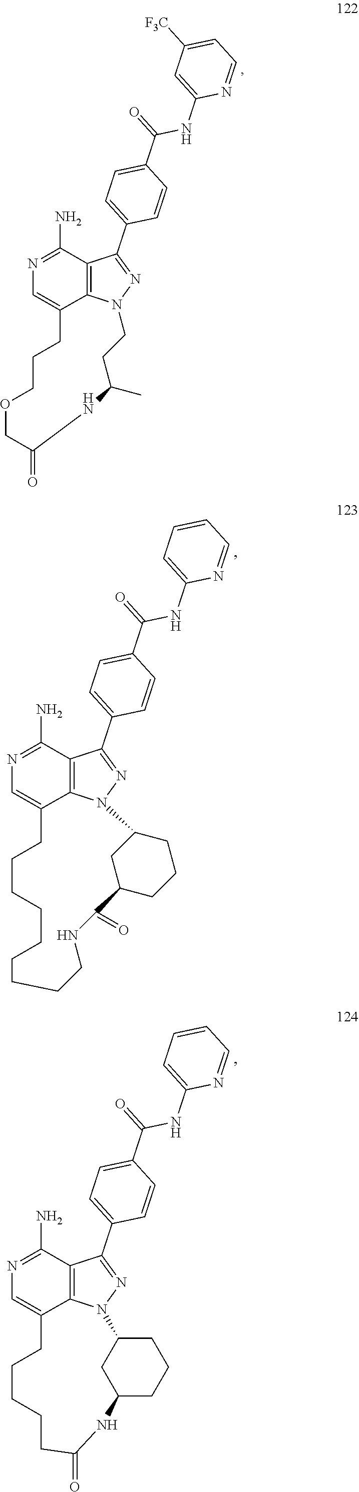 [SMILES: C[C@@H]1CCn2nc(-c3ccc(C(=O)Nc4cc(C(F)(F)F)ccn4)cc3)c3c(N)ncc(c32)CCCOCC(=O)N1 ; Nc1ncc2c3c1c(-c1ccc(C(=O)Nc4ccccn4)cc1)nn3[C@@H]1CCC[C@H](C1)C(=O)NCCCCCCCCC2 ; Nc1ncc2c3c1c(-c1ccc(C(=O)Nc4ccccn4)cc1)nn3[C@@H]1CCC[C@H](C1)NC(=O)CCCCC2]

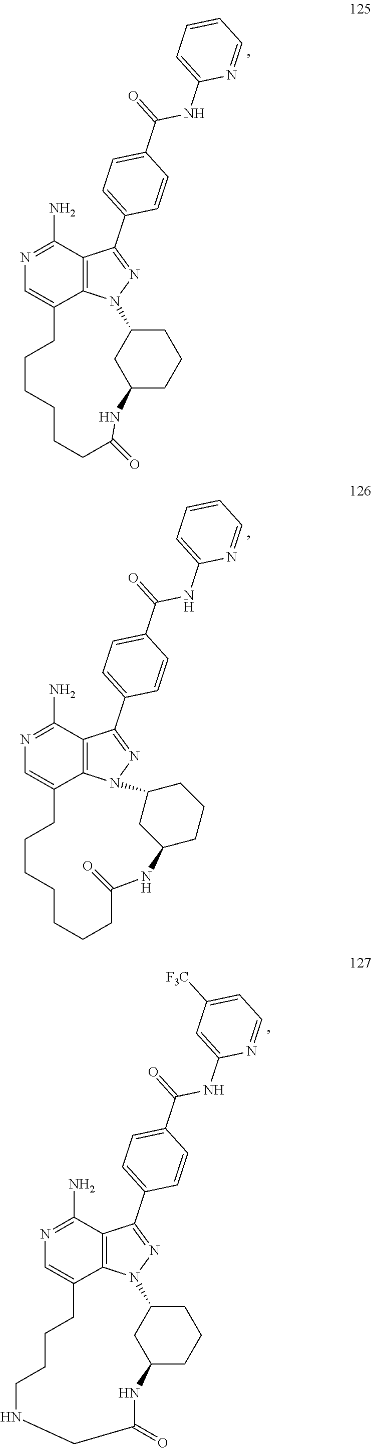 [SMILES: Nc1ncc2c3c1c(-c1ccc(C(=O)Nc4ccccn4)cc1)nn3[C@@H]1CCC[C@H](C1)NC(=O)CCCCCC2 ; Nc1ncc2c3c1c(-c1ccc(C(=O)Nc4ccccn4)cc1)nn3[C@@H]1CCC[C@H](C1)NC(=O)CCCCCCC2 ; Nc1ncc2c3c1c(-c1ccc(C(=O)Nc4cc(C(F)(F)F)ccn4)cc1)nn3[C@@H]1CCC[C@H](C1)NC(=O)CNCCCC2]

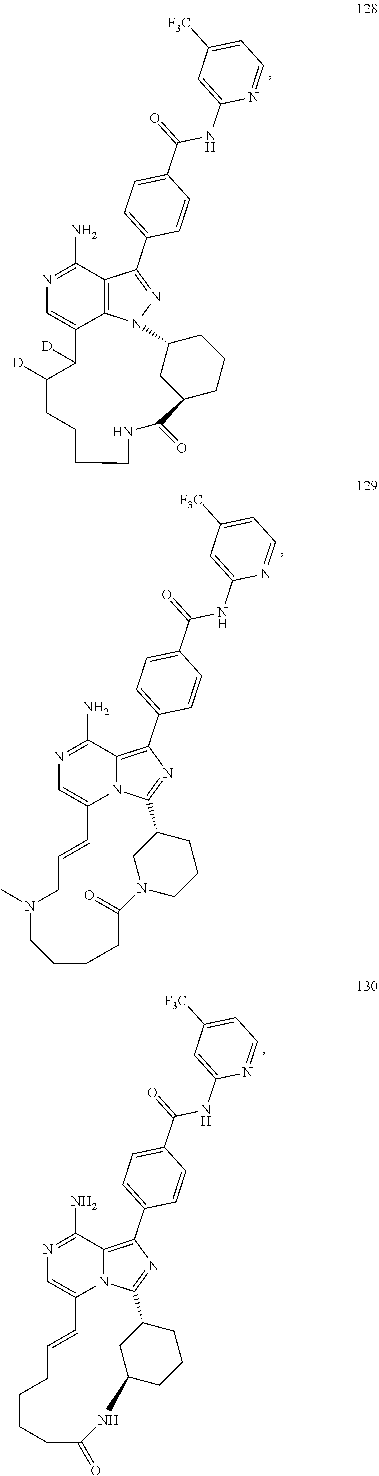 [SMILES: [2H]C1CCCCNC(=O)[C@@H]2CCC[C@H](C2)n2nc(-c3ccc(C(=O)Nc4cc(C(F)(F)F)ccn4)cc3)c3c(N)ncc(c32)C1[2H] ; CN1C/C=C/c2cnc(N)c3c(-c4ccc(C(=O)Nc5cc(C(F)(F)F)ccn5)cc4)nc(n23)[C@@H]2CCCN(C2)C(=O)CCCC1 ; Nc1ncc2n3c(nc(-c4ccc(C(=O)Nc5cc(C(F)(F)F)ccn5)cc4)c13)[C@@H]1CCC[C@H](C1)NC(=O)CCCC/C=C/2]

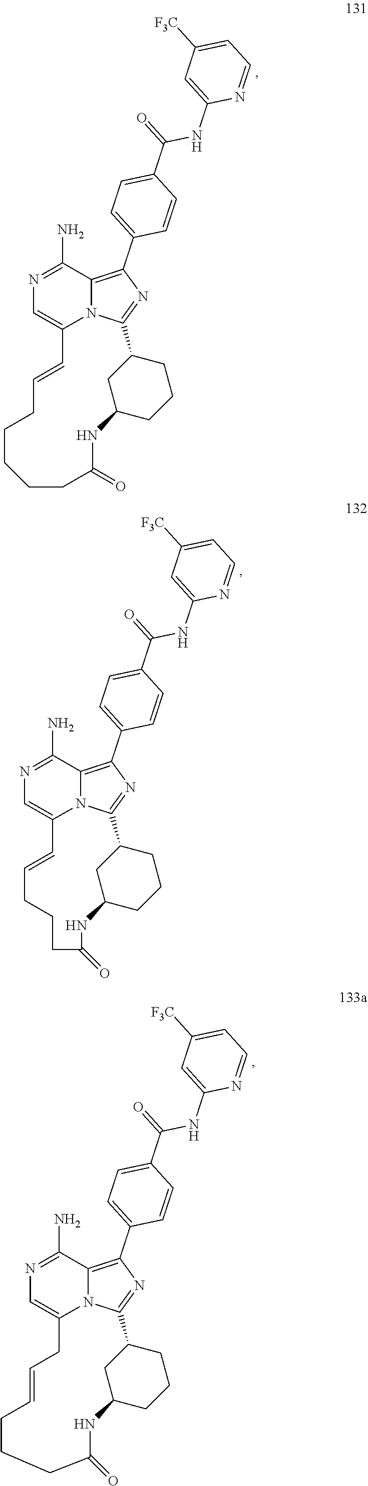 [SMILES: Nc1ncc2n3c(nc(-c4ccc(C(=O)Nc5cc(C(F)(F)F)ccn5)cc4)c13)[C@@H]1CCC[C@H](C1)NC(=O)CCCCC/C=C/2 ; Nc1ncc2n3c(nc(-c4ccc(C(=O)Nc5cc(C(F)(F)F)ccn5)cc4)c13)[C@@H]1CCC[C@H](C1)NC(=O)CCC/C=C/2 ; Nc1ncc2n3c(nc(-c4ccc(C(=O)Nc5cc(C(F)(F)F)ccn5)cc4)c13)[C@@H]1CCC[C@H](C1)NC(=O)CCC/C=C/C2]

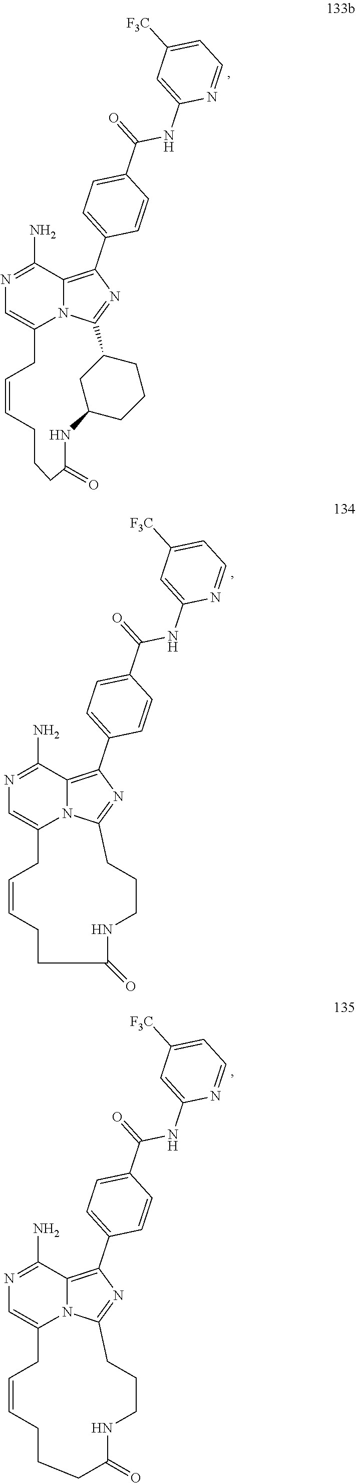

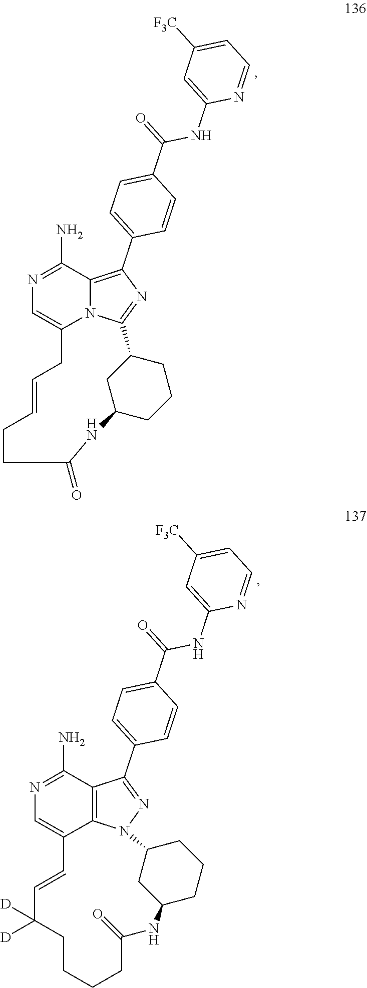 [SMILES: Nc1ncc2n3c(nc(-c4ccc(C(=O)Nc5cc(C(F)(F)F)ccn5)cc4)c13)[C@@H]1CCC[C@H](C1)NC(=O)CC/C=C/C2 ; [2H]C1([2H])/C=C/c2cnc(N)c3c(-c4ccc(C(=O)Nc5cc(C(F)(F)F)ccn5)cc4)nn(c23)[C@@H]2CCC[C@H](C2)NC(=O)CCCC1]

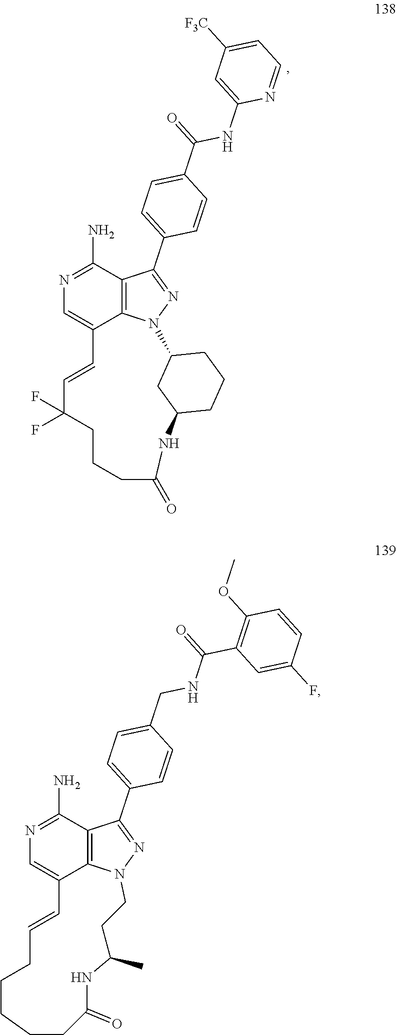 [SMILES: Nc1ncc2c3c1c(-c1ccc(C(=O)Nc4cc(C(F)(F)F)ccn4)cc1)nn3[C@@H]1CCC[C@H](C1)NC(=O)CCCC(F)(F)/C=C/2 ; COc1ccc(F)cc1C(=O)NCc1ccc(-c2nn3c4c(cnc(N)c24)/C=C/CCCCCC(=O)N[C@H](C)CC3)cc1]

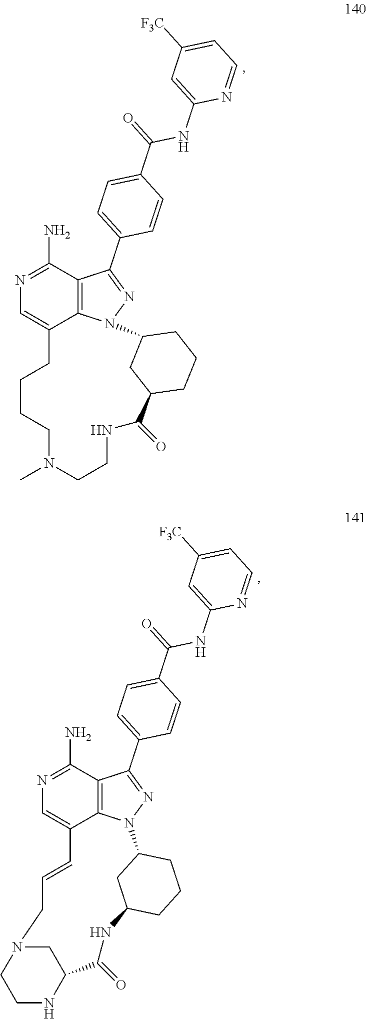 [SMILES: CN1CCCCc2cnc(N)c3c(-c4ccc(C(=O)Nc5cc(C(F)(F)F)ccn5)cc4)nn(c23)[C@@H]2CCC[C@H](C2)C(=O)NCC1 ; Nc1ncc2c3c1c(-c1ccc(C(=O)Nc4cc(C(F)(F)F)ccn4)cc1)nn3[C@@H]1CCC[C@H](C1)NC(=O)[C@H]1CN(C/C=C/2)CCN1]

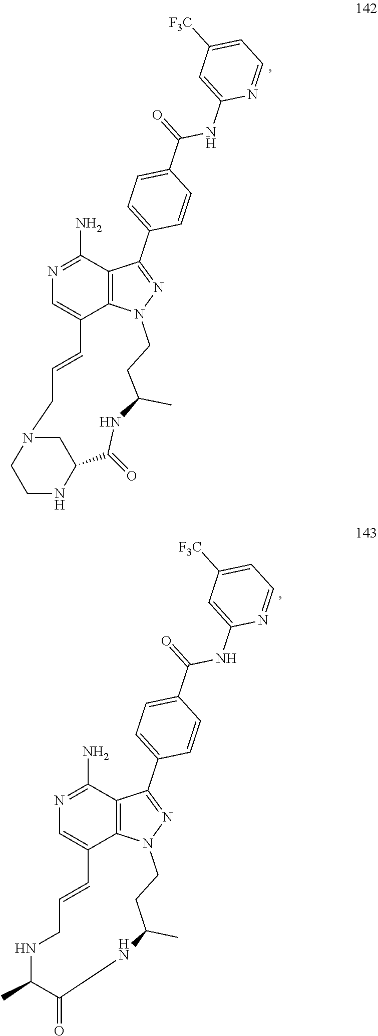 [SMILES: C[C@@H]1CCn2nc(-c3ccc(C(=O)Nc4cc(C(F)(F)F)ccn4)cc3)c3c(N)ncc(c32)/C=C/CN2CCN[C@H](C2)C(=O)N1 ; C[C@@H]1CCn2nc(-c3ccc(C(=O)Nc4cc(C(F)(F)F)ccn4)cc3)c3c(N)ncc(c32)/C=C/CN[C@H](C)C(=O)N1]

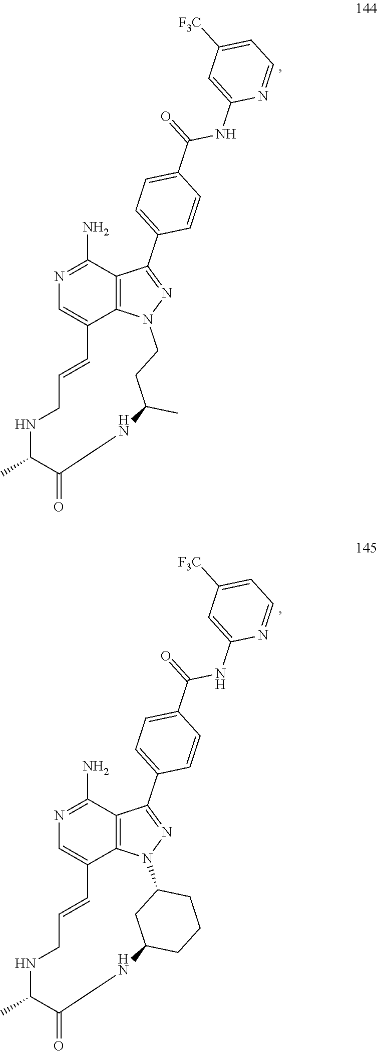 [SMILES: C[C@@H]1CCn2nc(-c3ccc(C(=O)Nc4cc(C(F)(F)F)ccn4)cc3)c3c(N)ncc(c32)/C=C/CN[C@@H](C)C(=O)N1 ; C[C@@H]1NC/C=C/c2cnc(N)c3c(-c4ccc(C(=O)Nc5cc(C(F)(F)F)ccn5)cc4)nn(c23)[C@@H]2CCC[C@H](C2)NC1=O]

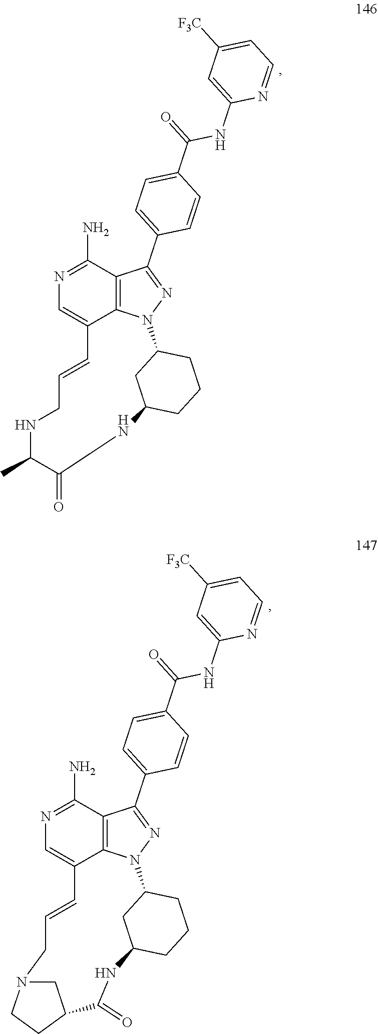 [SMILES: C[C@H]1NC/C=C/c2cnc(N)c3c(-c4ccc(C(=O)Nc5cc(C(F)(F)F)ccn5)cc4)nn(c23)[C@@H]2CCC[C@H](C2)NC1=O ; Nc1ncc2c3c1c(-c1ccc(C(=O)Nc4cc(C(F)(F)F)ccn4)cc1)nn3[C@@H]1CCC[C@H](C1)NC(=O)[C@@H]1CCN(C/C=C/2)C1]

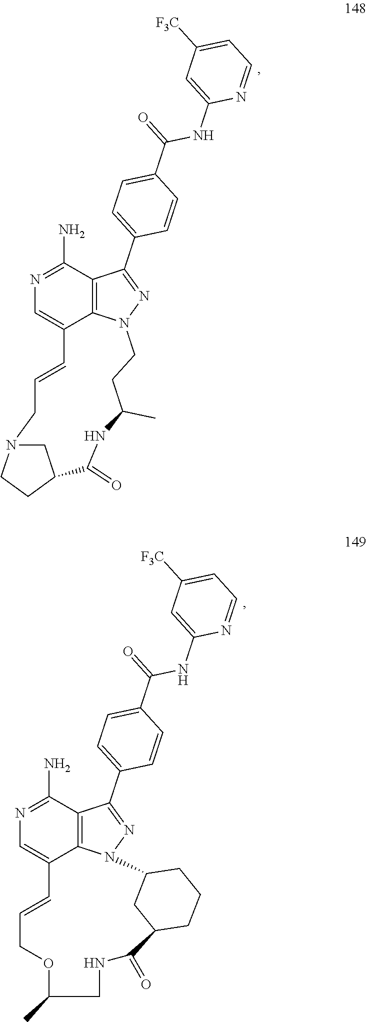 [SMILES: C[C@@H]1CCn2nc(-c3ccc(C(=O)Nc4cc(C(F)(F)F)ccn4)cc3)c3c(N)ncc(c32)/C=C/CN2CC[C@H](C2)C(=O)N1 ; C[C@@H]1CNC(=O)[C@@H]2CCC[C@H](C2)n2nc(-c3ccc(C(=O)Nc4cc(C(F)(F)F)ccn4)cc3)c3c(N)ncc(c32)/C=C/CO1]

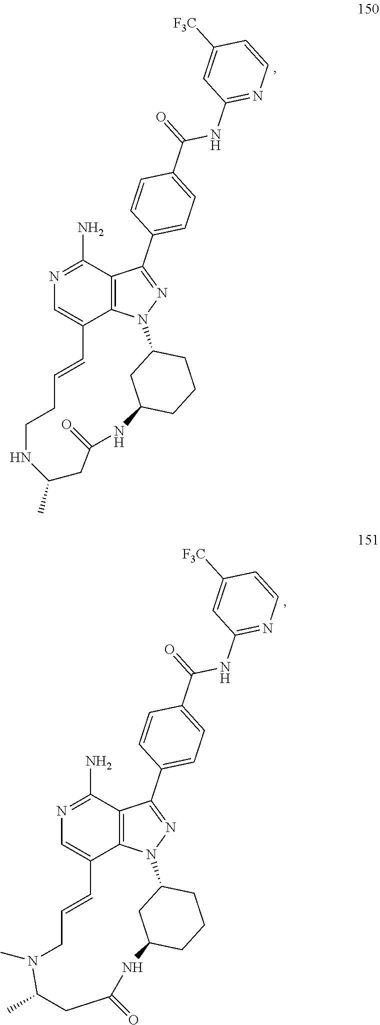 [SMILES: C[C@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCN1 ; C[C@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CN1C]

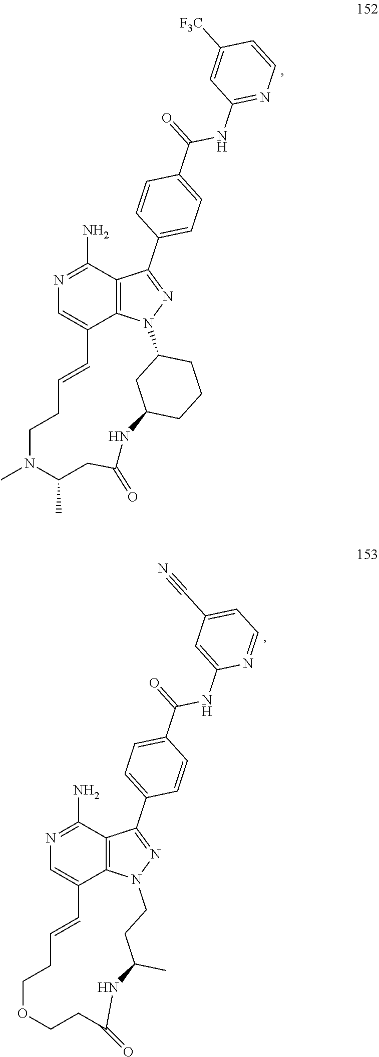 [SMILES: C[C@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCN1C ; C[C@@H]1CCn2nc(-c3ccc(C(=O)Nc4cc(C#N)ccn4)cc3)c3c(N)ncc(c32)/C=C/CCOCCC(=O)N1]

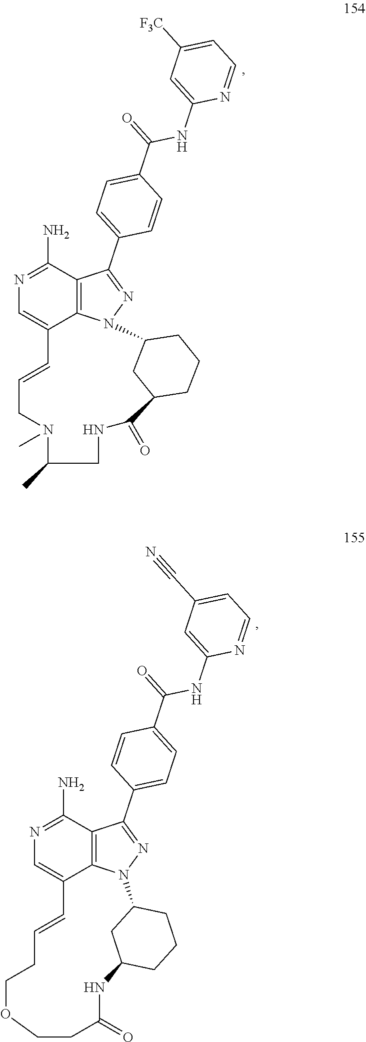 [SMILES: C[C@@H]1CNC(=O)[C@@H]2CCC[C@H](C2)n2nc(-c3ccc(C(=O)Nc4cc(C(F)(F)F)ccn4)cc3)c3c(N)ncc(c32)/C=C/CN1C ; N#Cc1ccnc(NC(=O)c2ccc(-c3nn4c5c(cnc(N)c35)/C=C/CCOCCC(=O)N[C@@H]3CCC[C@@H]4C3)cc2)c1]

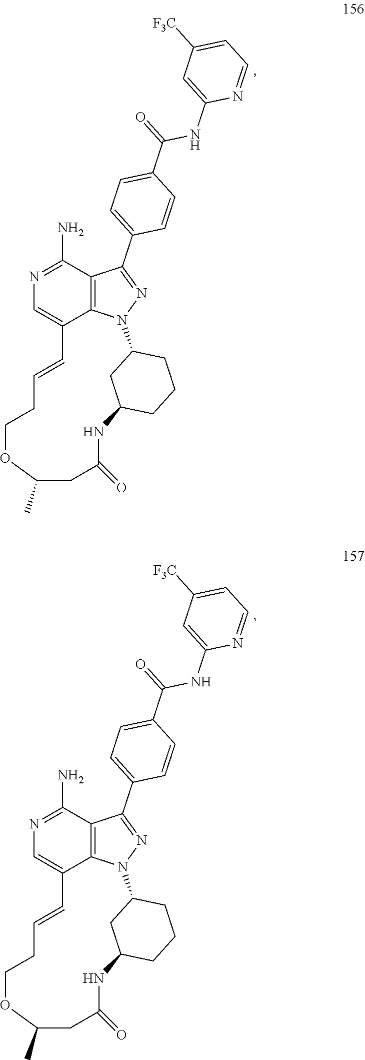 [SMILES: C[C@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCO1 ; C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCO1]

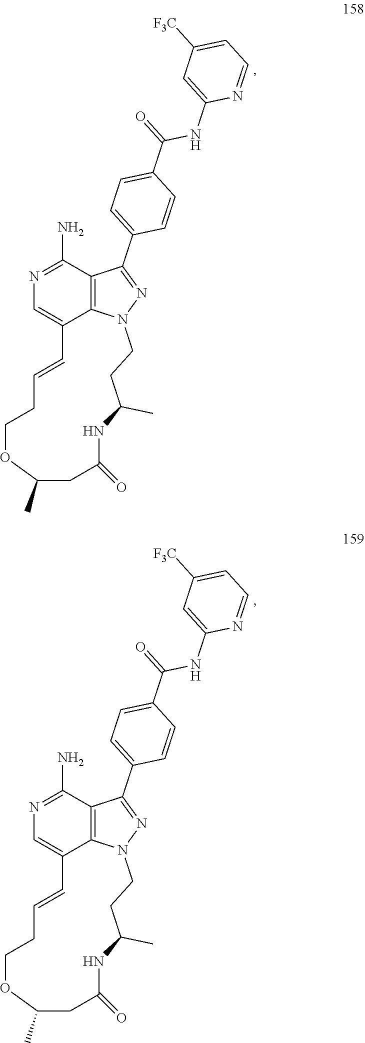 [SMILES: C[C@@H]1CCn2nc(-c3ccc(C(=O)Nc4cc(C(F)(F)F)ccn4)cc3)c3c(N)ncc(c32)/C=C/CCO[C@H](C)CC(=O)N1 ; C[C@@H]1CCn2nc(-c3ccc(C(=O)Nc4cc(C(F)(F)F)ccn4)cc3)c3c(N)ncc(c32)/C=C/CCO[C@@H](C)CC(=O)N1]

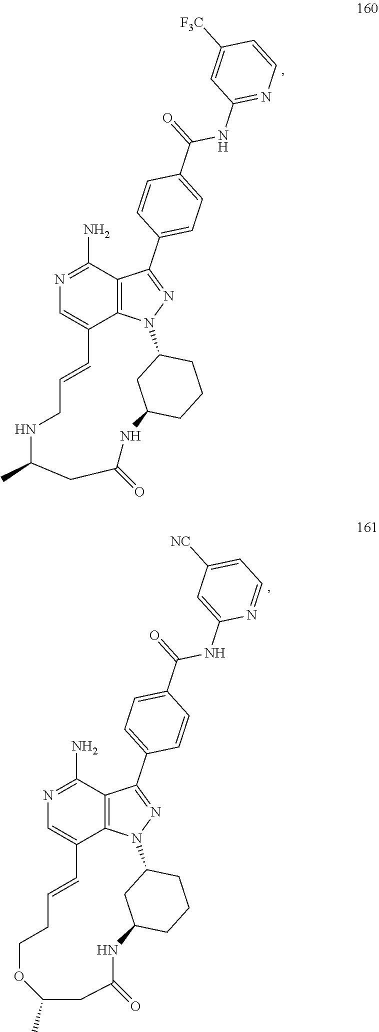 [SMILES: C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CN1 ; C[C@H]1CC(=O)N[C@@H]2CCC[C@H](C2)n2nc(-c3ccc(C(=O)Nc4cc(C#N)ccn4)cc3)c3c(N)ncc(c32)/C=C/CCO1]

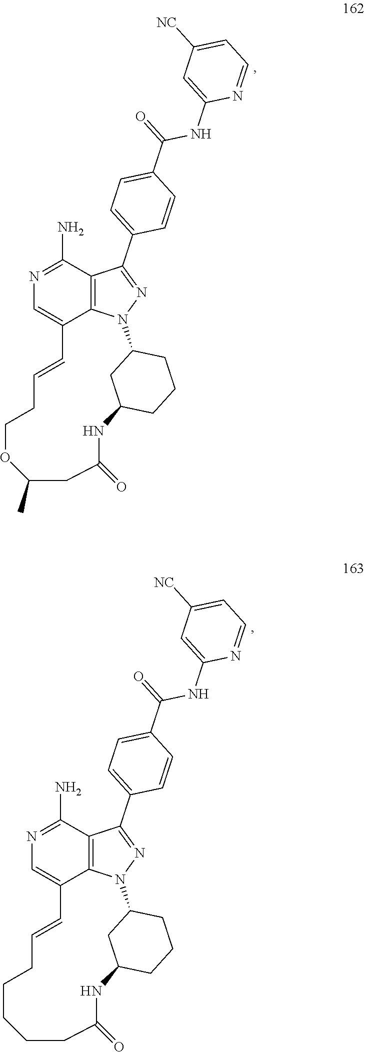 [SMILES: C[C@@H]1CC(=O)N[C@@H]2CCC[C@H](C2)n2nc(-c3ccc(C(=O)Nc4cc(C#N)ccn4)cc3)c3c(N)ncc(c32)/C=C/CCO1 ; N#Cc1ccnc(NC(=O)c2ccc(-c3nn4c5c(cnc(N)c35)/C=C/CCCCCC(=O)N[C@@H]3CCC[C@@H]4C3)cc2)c1]

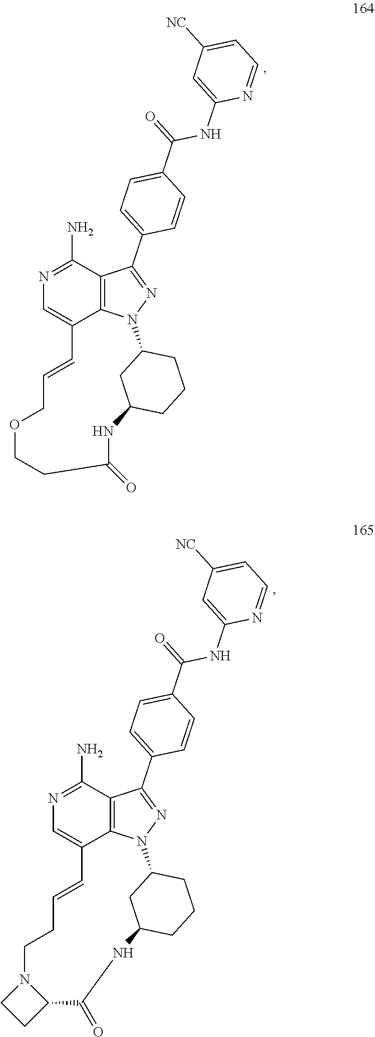 [SMILES: N#Cc1ccnc(NC(=O)c2ccc(-c3nn4c5c(cnc(N)c35)/C=C/COCCC(=O)N[C@@H]3CCC[C@@H]4C3)cc2)c1 ; N#Cc1ccnc(NC(=O)c2ccc(-c3nn4c5c(cnc(N)c35)/C=C/CCN3CC[C@H]3C(=O)N[C@@H]3CCC[C@@H]4C3)cc2)c1]

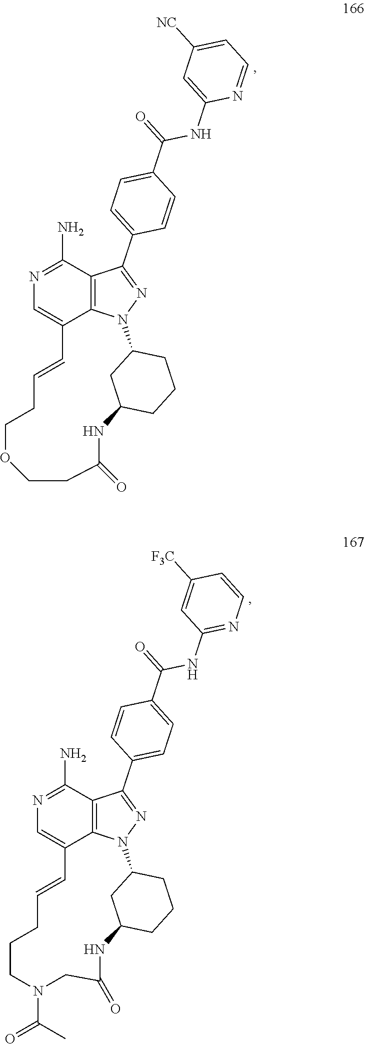 [SMILES: N#Cc1ccnc(NC(=O)c2ccc(-c3nn4c5c(cnc(N)c35)/C=C/CCOCCC(=O)N[C@@H]3CCC[C@@H]4C3)cc2)c1 ; CC(=O)N1CCC/C=C/c2cnc(N)c3c(-c4ccc(C(=O)Nc5cc(C(F)(F)F)ccn5)cc4)nn(c23)[C@@H]2CCC[C@H](C2)NC(=O)C1]

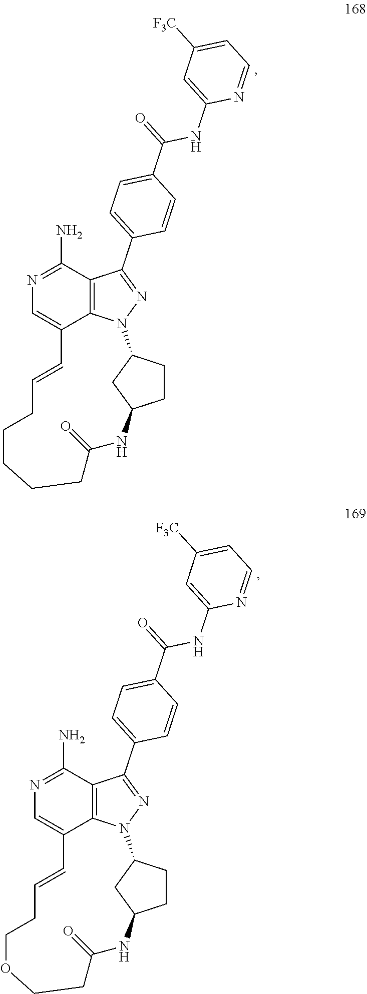 [SMILES: Nc1ncc2c3c1c(-c1ccc(C(=O)Nc4cc(C(F)(F)F)ccn4)cc1)nn3[C@@H]1CC[C@H](C1)NC(=O)CCCCC/C=C/2 ; Nc1ncc2c3c1c(-c1ccc(C(=O)Nc4cc(C(F)(F)F)ccn4)cc1)nn3[C@@H]1CC[C@H](C1)NC(=O)CCOCC/C=C/2]

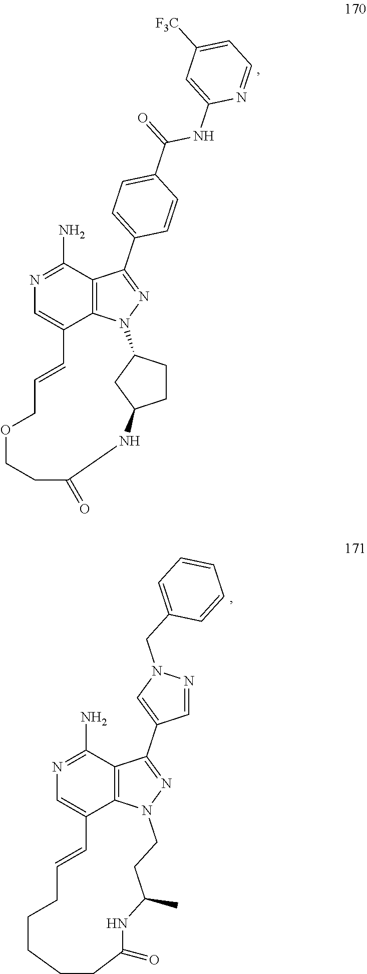 [SMILES: Nc1ncc2c3c1c(-c1ccc(C(=O)Nc4cc(C(F)(F)F)ccn4)cc1)nn3[C@@H]1CC[C@H](C1)NC(=O)CCOC/C=C/2 ; C[C@@H]1CCn2nc(-c3cnn(Cc4ccccc4)c3)c3c(N)ncc(c32)/C=C/CCCCCC(=O)N1]

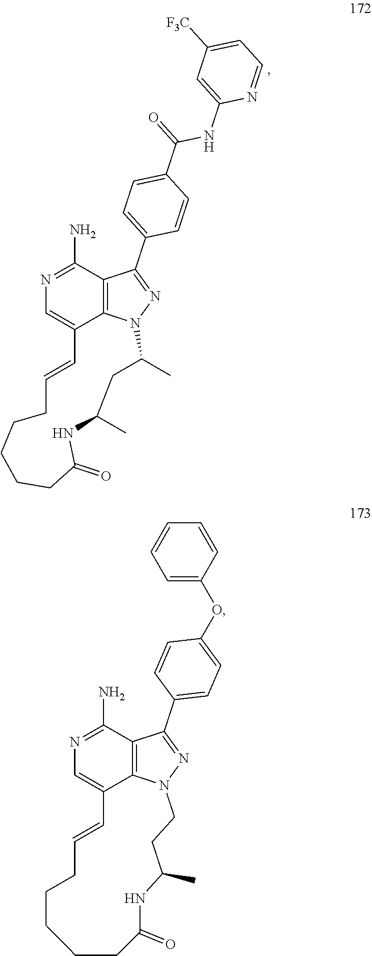 [SMILES: C[C@@H]1C[C@@H](C)n2nc(-c3ccc(C(=O)Nc4cc(C(F)(F)F)ccn4)cc3)c3c(N)ncc(c32)/C=C/CCCCCC(=O)N1 ; C[C@@H]1CCn2nc(-c3ccc(Oc4ccccc4)cc3)c3c(N)ncc(c32)/C=C/CCCCCC(=O)N1]

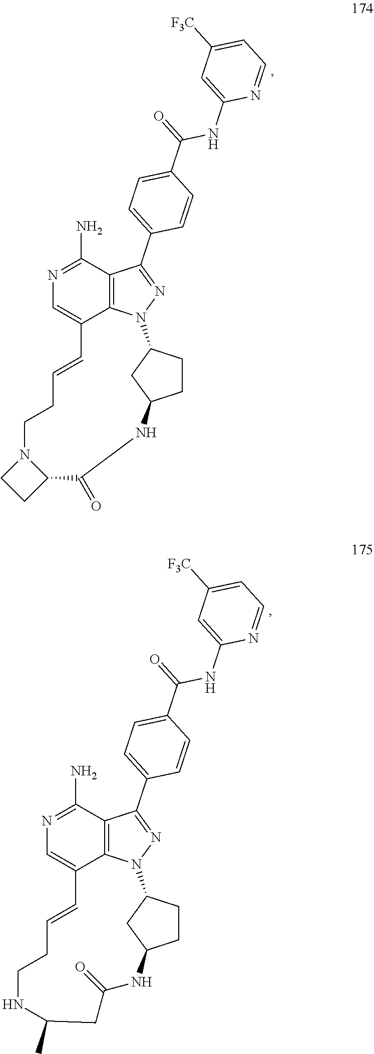 [SMILES: Nc1ncc2c3c1c(-c1ccc(C(=O)Nc4cc(C(F)(F)F)ccn4)cc1)nn3[C@@H]1CC[C@H](C1)NC(=O)[C@@H]1CCN1CC/C=C/2 ; C[C@@H]1CC(=O)N[C@@H]2CC[C@H](C2)n2nc(-c3ccc(C(=O)Nc4cc(C(F)(F)F)ccn4)cc3)c3c(N)ncc(c32)/C=C/CCN1]

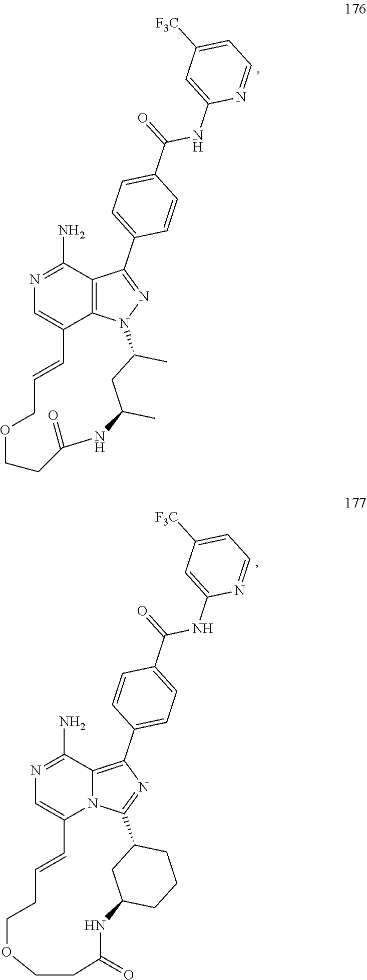 [SMILES: C[C@@H]1C[C@@H](C)n2nc(-c3ccc(C(=O)Nc4cc(C(F)(F)F)ccn4)cc3)c3c(N)ncc(c32)/C=C/COCCC(=O)N1 ; Nc1ncc2n3c(nc(-c4ccc(C(=O)Nc5cc(C(F)(F)F)ccn5)cc4)c13)[C@@H]1CCC[C@H](C1)NC(=O)CCOCC/C=C/2]

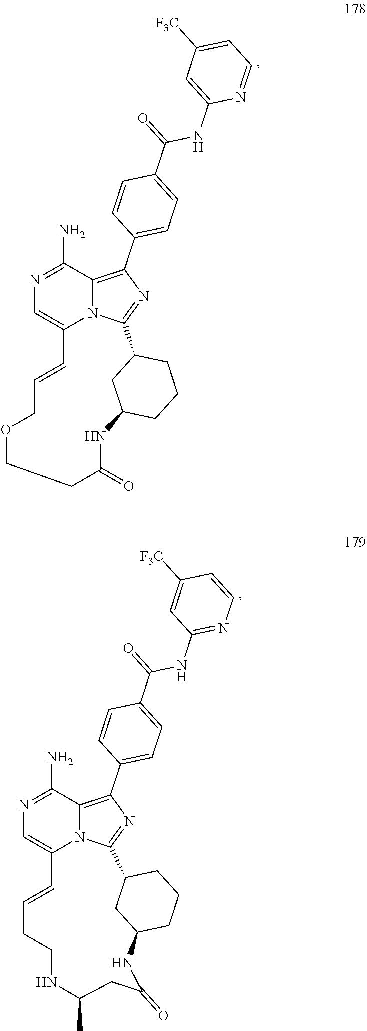 [SMILES: Nc1ncc2n3c(nc(-c4ccc(C(=O)Nc5cc(C(F)(F)F)ccn5)cc4)c13)[C@@H]1CCC[C@H](C1)NC(=O)CCOC/C=C/2 ; C[C@@H]1CC(=O)N[C@@H]2CCC[C@H](C2)c2nc(-c3ccc(C(=O)Nc4cc(C(F)(F)F)ccn4)cc3)c3c(N)ncc(n23)/C=C/CCN1]

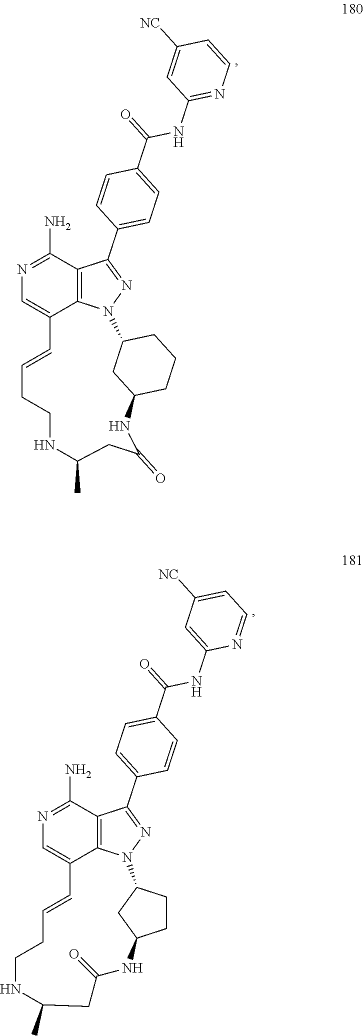 [SMILES: C[C@@H]1CC(=O)N[C@@H]2CCC[C@H](C2)n2nc(-c3ccc(C(=O)Nc4cc(C#N)ccn4)cc3)c3c(N)ncc(c32)/C=C/CCN1 ; C[C@@H]1CC(=O)N[C@@H]2CC[C@H](C2)n2nc(-c3ccc(C(=O)Nc4cc(C#N)ccn4)cc3)c3c(N)ncc(c32)/C=C/CCN1]

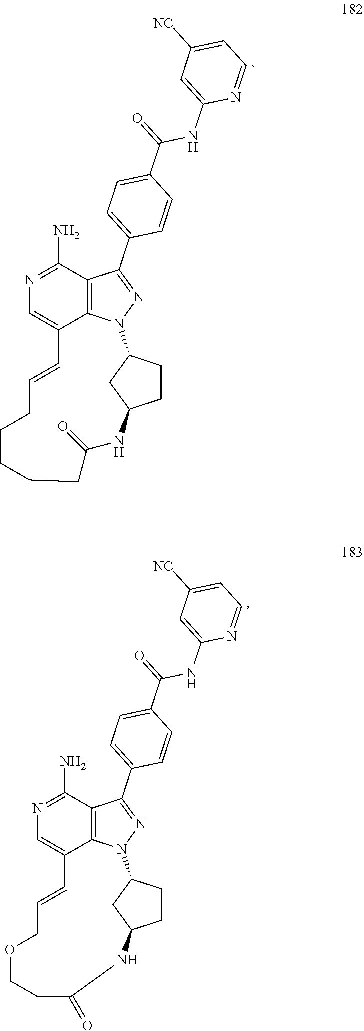 [SMILES: N#Cc1ccnc(NC(=O)c2ccc(-c3nn4c5c(cnc(N)c35)/C=C/CCCCCC(=O)N[C@@H]3CC[C@@H]4C3)cc2)c1 ; N#Cc1ccnc(NC(=O)c2ccc(-c3nn4c5c(cnc(N)c35)/C=C/COCCC(=O)N[C@@H]3CC[C@@H]4C3)cc2)c1]

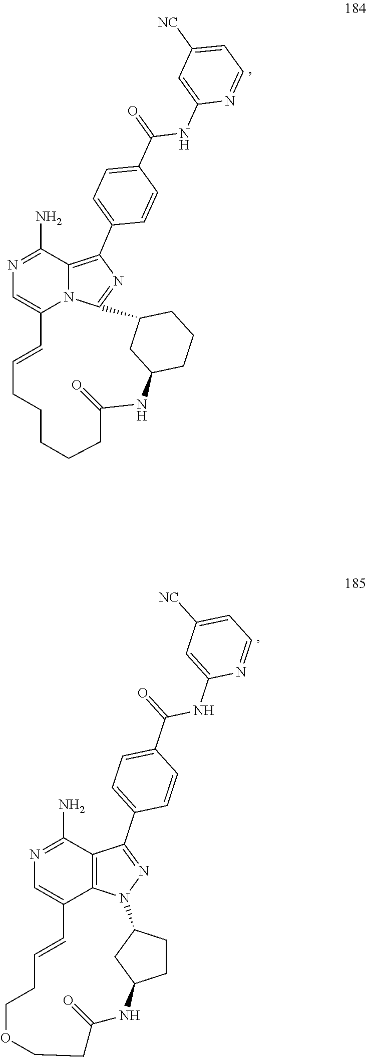 [SMILES: N#Cc1ccnc(NC(=O)c2ccc(-c3nc4n5c(cnc(N)c35)/C=C/CCCCCC(=O)N[C@@H]3CCC[C@@H]4C3)cc2)c1 ; N#Cc1ccnc(NC(=O)c2ccc(-c3nn4c5c(cnc(N)c35)/C=C/CCOCCC(=O)N[C@@H]3CC[C@@H]4C3)cc2)c1]

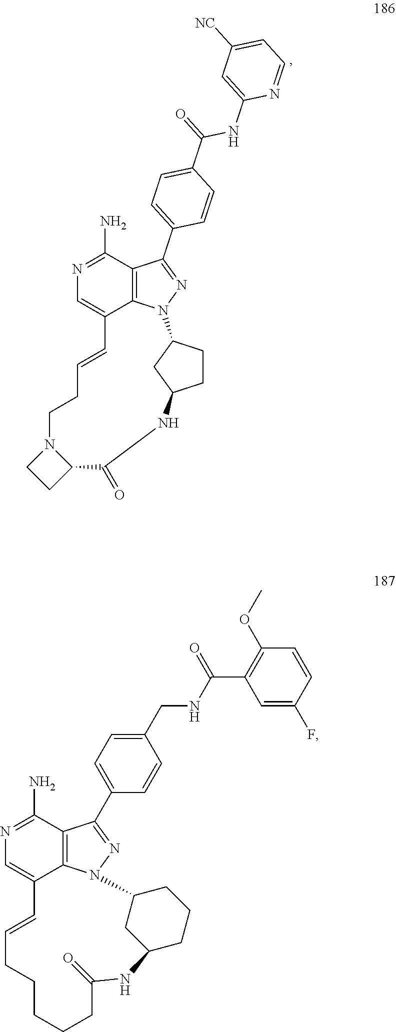 [SMILES: N#Cc1ccnc(NC(=O)c2ccc(-c3nn4c5c(cnc(N)c35)/C=C/CCN3CC[C@H]3C(=O)N[C@@H]3CC[C@@H]4C3)cc2)c1 ; COc1ccc(F)cc1C(=O)NCc1ccc(-c2nn3c4c(cnc(N)c24)/C=C/CCCCCC(=O)N[C@@H]2CCC[C@@H]3C2)cc1]

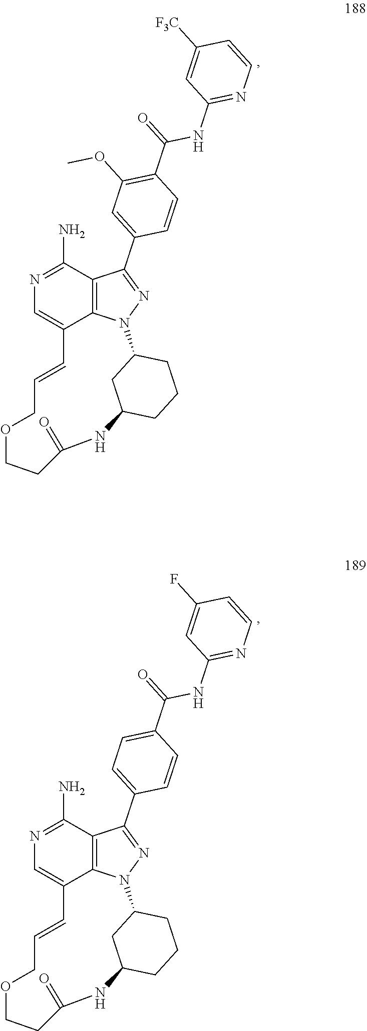 [SMILES: COc1cc(-c2nn3c4c(cnc(N)c24)/C=C/COCCC(=O)N[C@@H]2CCC[C@@H]3C2)ccc1C(=O)Nc1cc(C(F)(F)F)ccn1 ; Nc1ncc2c3c1c(-c1ccc(C(=O)Nc4cc(F)ccn4)cc1)nn3[C@@H]1CCC[C@H](C1)NC(=O)CCOC/C=C/2]

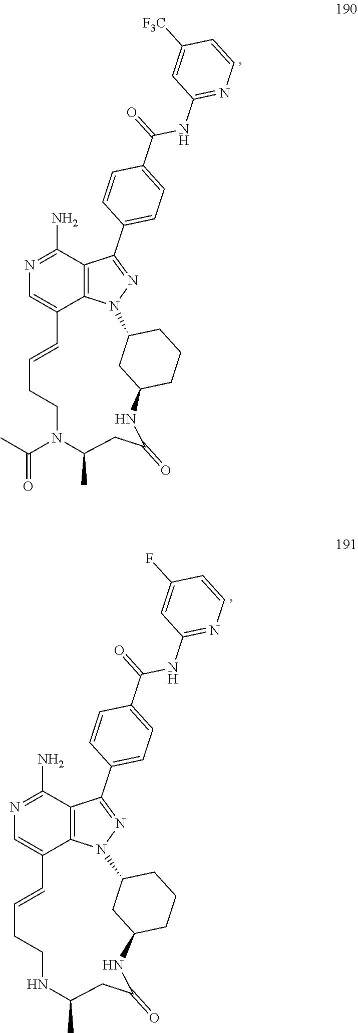 [SMILES: CC(=O)N1CC/C=C/c2cnc(N)c3c(-c4ccc(C(=O)Nc5cc(C(F)(F)F)ccn5)cc4)nn(c23)[C@@H]2CCC[C@H](C2)NC(=O)C[C@H]1C ; C[C@@H]1CC(=O)N[C@@H]2CCC[C@H](C2)n2nc(-c3ccc(C(=O)Nc4cc(F)ccn4)cc3)c3c(N)ncc(c32)/C=C/CCN1]

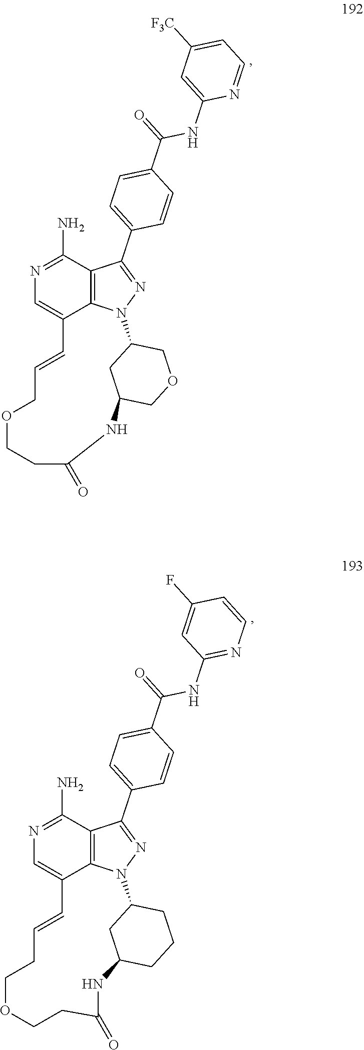 [SMILES: Nc1ncc2c3c1c(-c1ccc(C(=O)Nc4cc(C(F)(F)F)ccn4)cc1)nn3[C@@H]1COC[C@H](C1)NC(=O)CCOC/C=C/2 ; Nc1ncc2c3c1c(-c1ccc(C(=O)Nc4cc(F)ccn4)cc1)nn3[C@@H]1CCC[C@H](C1)NC(=O)CCOCC/C=C/2]

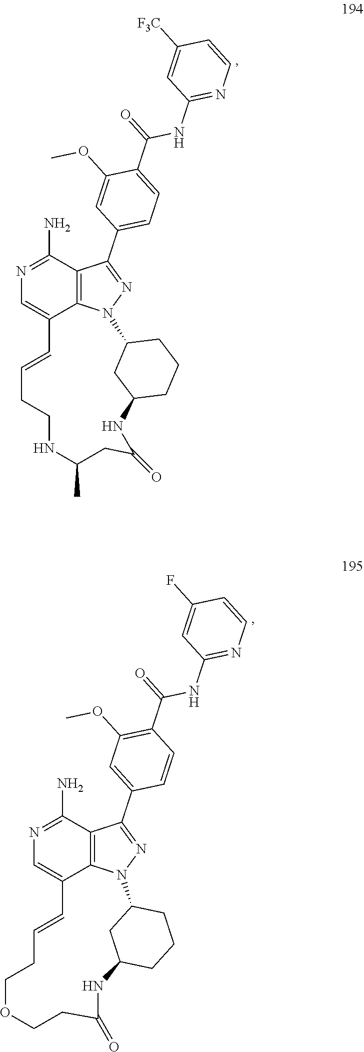 [SMILES: COc1cc(-c2nn3c4c(cnc(N)c24)/C=C/CCN[C@H](C)CC(=O)N[C@@H]2CCC[C@@H]3C2)ccc1C(=O)Nc1cc(C(F)(F)F)ccn1 ; COc1cc(-c2nn3c4c(cnc(N)c24)/C=C/CCOCCC(=O)N[C@@H]2CCC[C@@H]3C2)ccc1C(=O)Nc1cc(F)ccn1]

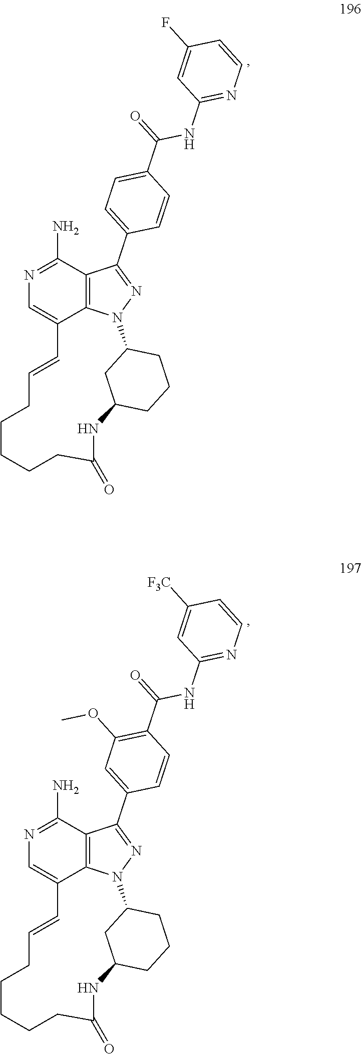 [SMILES: Nc1ncc2c3c1c(-c1ccc(C(=O)Nc4cc(F)ccn4)cc1)nn3[C@@H]1CCC[C@H](C1)NC(=O)CCCCC/C=C/2 ; COc1cc(-c2nn3c4c(cnc(N)c24)/C=C/CCCCCC(=O)N[C@@H]2CCC[C@@H]3C2)ccc1C(=O)Nc1cc(C(F)(F)F)ccn1]

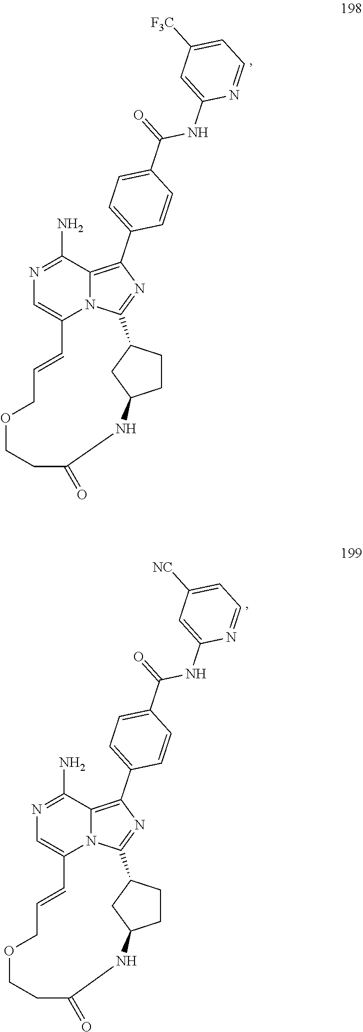 [SMILES: Nc1ncc2n3c(nc(-c4ccc(C(=O)Nc5cc(C(F)(F)F)ccn5)cc4)c13)[C@@H]1CC[C@H](C1)NC(=O)CCOC/C=C/2 ; N#Cc1ccnc(NC(=O)c2ccc(-c3nc4n5c(cnc(N)c35)/C=C/COCCC(=O)N[C@@H]3CC[C@@H]4C3)cc2)c1]

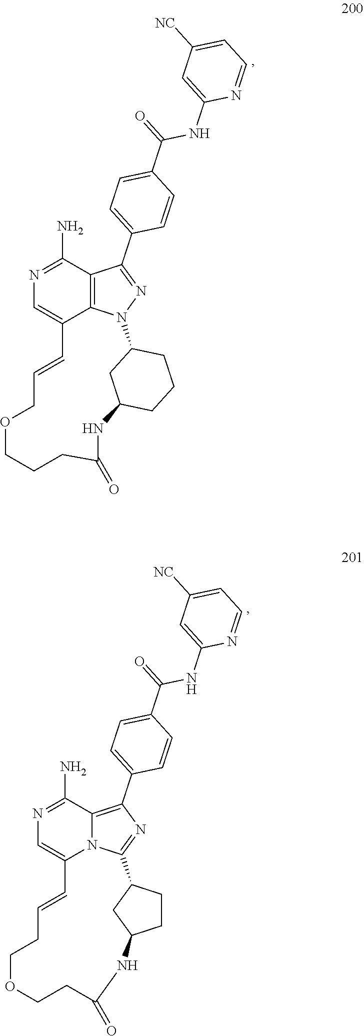 [SMILES: N#Cc1ccnc(NC(=O)c2ccc(-c3nn4c5c(cnc(N)c35)/C=C/COCCCC(=O)N[C@@H]3CCC[C@@H]4C3)cc2)c1 ; N#Cc1ccnc(NC(=O)c2ccc(-c3nc4n5c(cnc(N)c35)/C=C/CCOCCC(=O)N[C@@H]3CC[C@@H]4C3)cc2)c1]

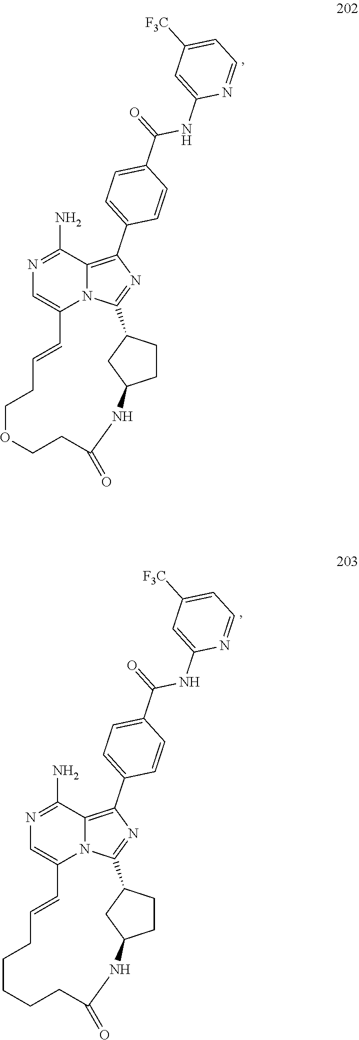 [SMILES: Nc1ncc2n3c(nc(-c4ccc(C(=O)Nc5cc(C(F)(F)F)ccn5)cc4)c13)[C@@H]1CC[C@H](C1)NC(=O)CCOCC/C=C/2 ; Nc1ncc2n3c(nc(-c4ccc(C(=O)Nc5cc(C(F)(F)F)ccn5)cc4)c13)[C@@H]1CC[C@H](C1)NC(=O)CCCCC/C=C/2]

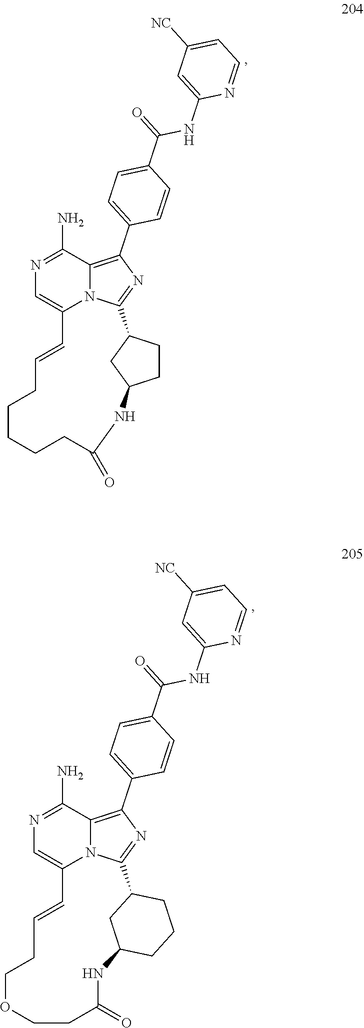 [SMILES: N#Cc1ccnc(NC(=O)c2ccc(-c3nc4n5c(cnc(N)c35)/C=C/CCCCCC(=O)N[C@@H]3CC[C@@H]4C3)cc2)c1 ; N#Cc1ccnc(NC(=O)c2ccc(-c3nc4n5c(cnc(N)c35)/C=C/CCOCCC(=O)N[C@@H]3CCC[C@@H]4C3)cc2)c1]

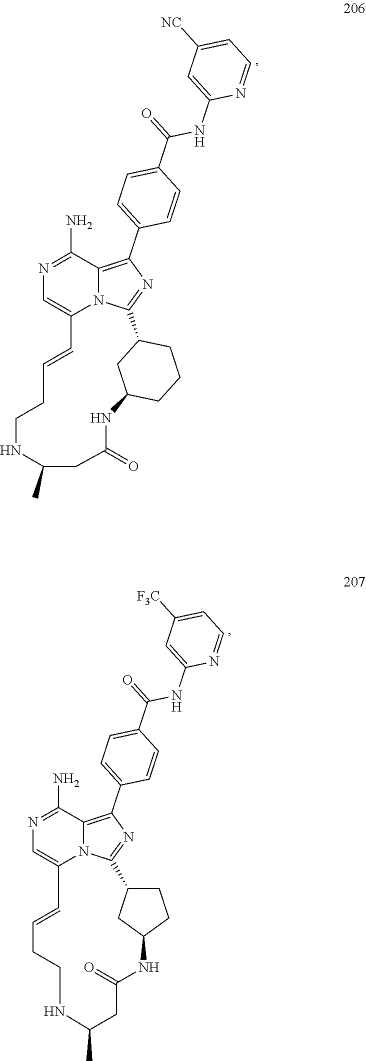 [SMILES: C[C@@H]1CC(=O)N[C@@H]2CCC[C@H](C2)c2nc(-c3ccc(C(=O)Nc4cc(C#N)ccn4)cc3)c3c(N)ncc(n23)/C=C/CCN1 ; C[C@@H]1CC(=O)N[C@@H]2CC[C@H](C2)c2nc(-c3ccc(C(=O)Nc4cc(C(F)(F)F)ccn4)cc3)c3c(N)ncc(n23)/C=C/CCN1]

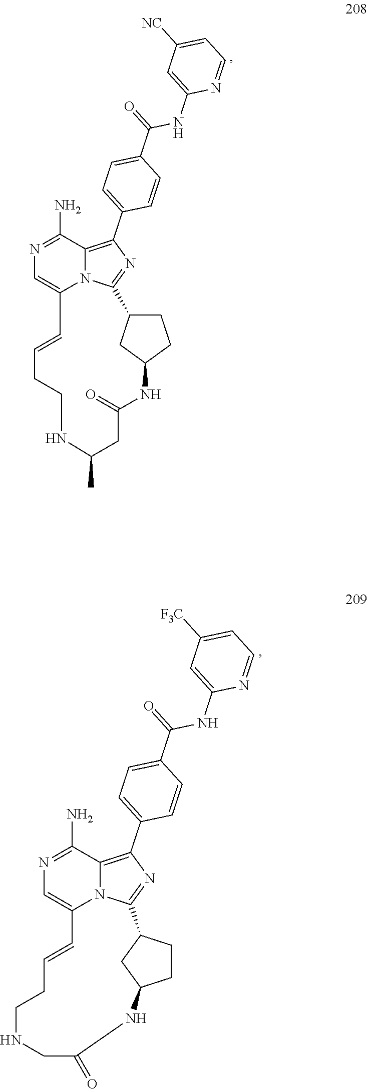 [SMILES: C[C@@H]1CC(=O)N[C@@H]2CC[C@H](C2)c2nc(-c3ccc(C(=O)Nc4cc(C#N)ccn4)cc3)c3c(N)ncc(n23)/C=C/CCN1 ; Nc1ncc2n3c(nc(-c4ccc(C(=O)Nc5cc(C(F)(F)F)ccn5)cc4)c13)[C@@H]1CC[C@H](C1)NC(=O)CNCC/C=C/2]

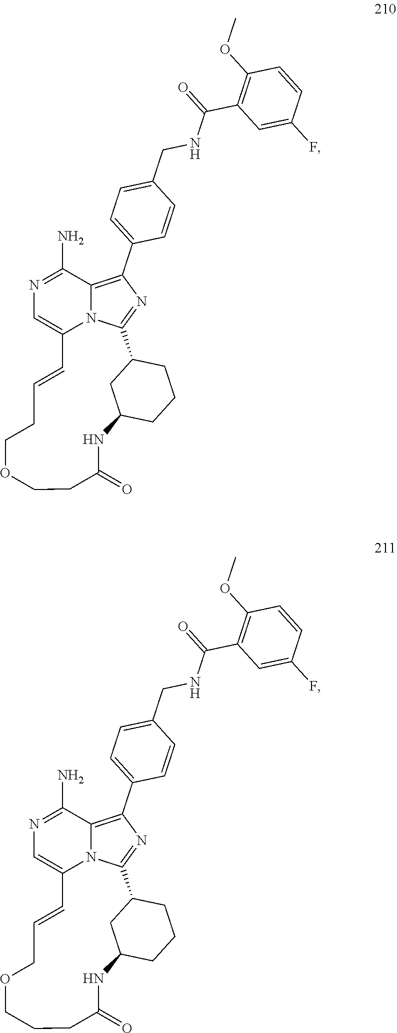 [SMILES: COc1ccc(F)cc1C(=O)NCc1ccc(-c2nc3n4c(cnc(N)c24)/C=C/CCOCCC(=O)N[C@@H]2CCC[C@@H]3C2)cc1 ; COc1ccc(F)cc1C(=O)NCc1ccc(-c2nc3n4c(cnc(N)c24)/C=C/COCCCC(=O)N[C@@H]2CCC[C@@H]3C2)cc1]

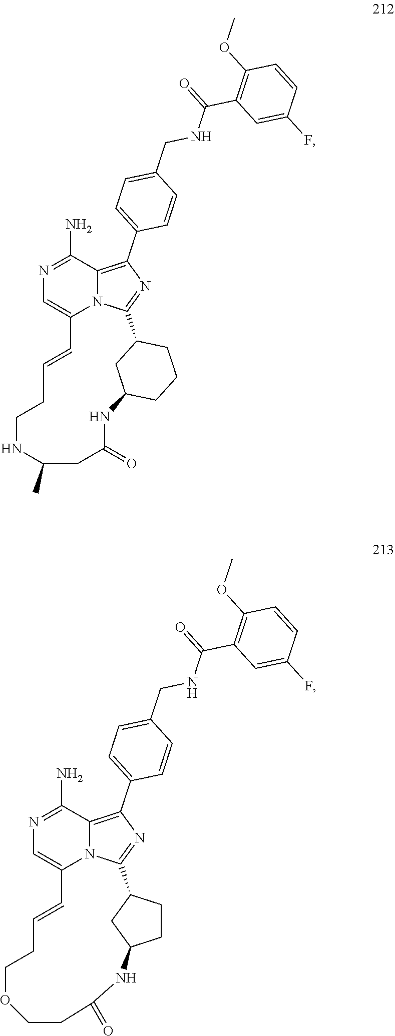 [SMILES: COc1ccc(F)cc1C(=O)NCc1ccc(-c2nc3n4c(cnc(N)c24)/C=C/CCN[C@H](C)CC(=O)N[C@@H]2CCC[C@@H]3C2)cc1 ; COc1ccc(F)cc1C(=O)NCc1ccc(-c2nc3n4c(cnc(N)c24)/C=C/CCOCCC(=O)N[C@@H]2CC[C@@H]3C2)cc1]

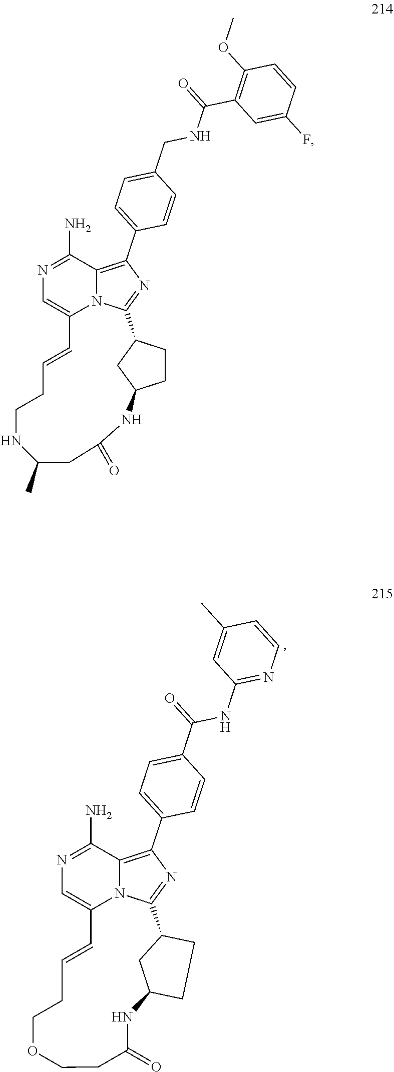 [SMILES: COc1ccc(F)cc1C(=O)NCc1ccc(-c2nc3n4c(cnc(N)c24)/C=C/CCN[C@H](C)CC(=O)N[C@@H]2CC[C@@H]3C2)cc1 ; Cc1ccnc(NC(=O)c2ccc(-c3nc4n5c(cnc(N)c35)/C=C/CCOCCC(=O)N[C@@H]3CC[C@@H]4C3)cc2)c1]

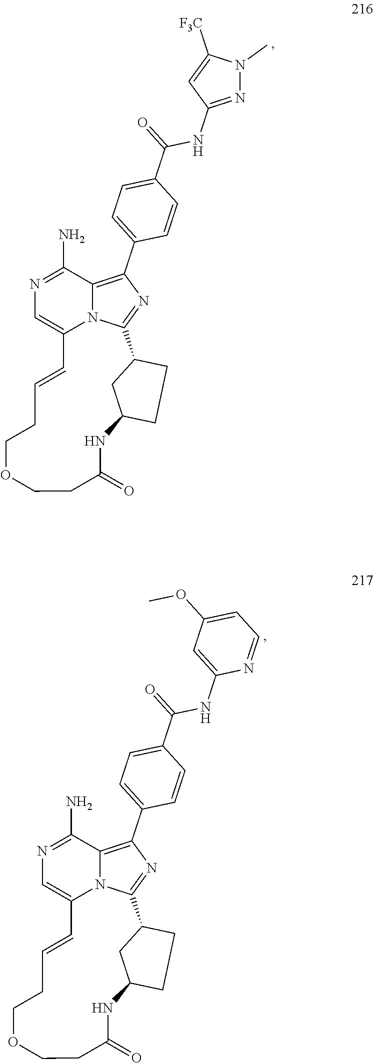 [SMILES: Cn1nc(NC(=O)c2ccc(-c3nc4n5c(cnc(N)c35)/C=C/CCOCCC(=O)N[C@@H]3CC[C@@H]4C3)cc2)cc1C(F)(F)F ; COc1ccnc(NC(=O)c2ccc(-c3nc4n5c(cnc(N)c35)/C=C/CCOCCC(=O)N[C@@H]3CC[C@@H]4C3)cc2)c1]

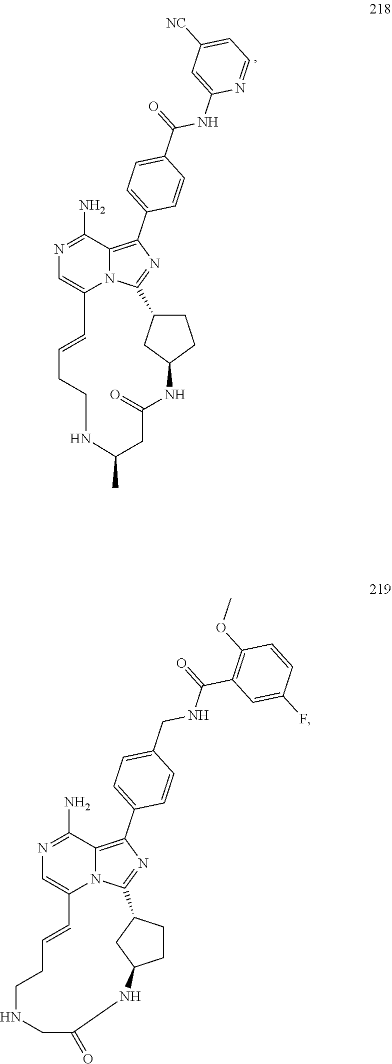 [SMILES: C[C@@H]1CC(=O)N[C@@H]2CC[C@H](C2)c2nc(-c3ccc(C(=O)Nc4cc(C#N)ccn4)cc3)c3c(N)ncc(n23)/C=C/CCN1 ; COc1ccc(F)cc1C(=O)NCc1ccc(-c2nc3n4c(cnc(N)c24)/C=C/CCNCC(=O)N[C@@H]2CC[C@@H]3C2)cc1]

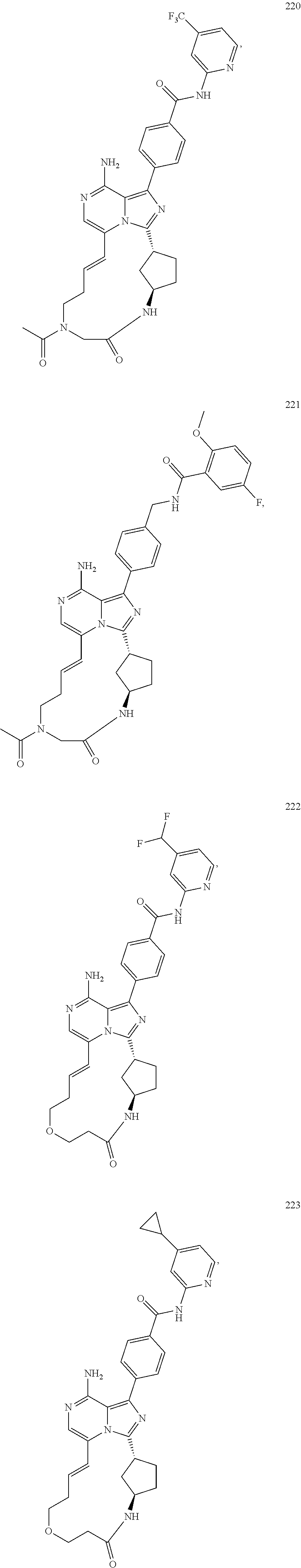 [SMILES: CC(=O)N1CC/C=C/c2cnc(N)c3c(-c4ccc(C(=O)Nc5cc(C(F)(F)F)ccn5)cc4)nc(n23)[C@@H]2CC[C@H](C2)NC(=O)C1 ; COc1ccc(F)cc1C(=O)NCc1ccc(-c2nc3n4c(cnc(N)c24)/C=C/CCN(C(C)=O)CC(=O)N[C@@H]2CC[C@@H]3C2)cc1 ; Nc1ncc2n3c(nc(-c4ccc(C(=O)Nc5cc(C(F)F)ccn5)cc4)c13)[C@@H]1CC[C@H](C1)NC(=O)CCOCC/C=C/2 ; Nc1ncc2n3c(nc(-c4ccc(C(=O)Nc5cc(C6CC6)ccn5)cc4)c13)[C@@H]1CC[C@H](C1)NC(=O)CCOCC/C=C/2]

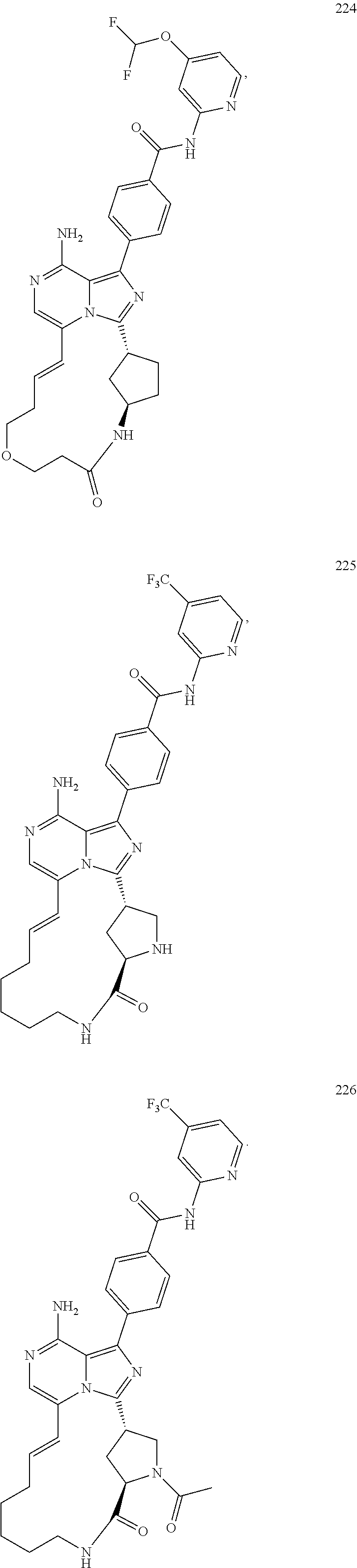 [SMILES: Nc1ncc2n3c(nc(-c4ccc(C(=O)Nc5cc(OC(F)F)ccn5)cc4)c13)[C@@H]1CC[C@H](C1)NC(=O)CCOCC/C=C/2 ; Nc1ncc2n3c(nc(-c4ccc(C(=O)Nc5cc(C(F)(F)F)ccn5)cc4)c13)[C@@H]1CN[C@H](C1)C(=O)NCCCCC/C=C/2 ; CC(=O)N1C[C@@H]2C[C@@H]1C(=O)NCCCCC/C=C/c1cnc(N)c3c(-c4ccc(C(=O)Nc5cc(C(F)(F)F)ccn5)cc4)nc2n13]

39. The compound according to claim 32, wherein the compound is selected from the group consisting of

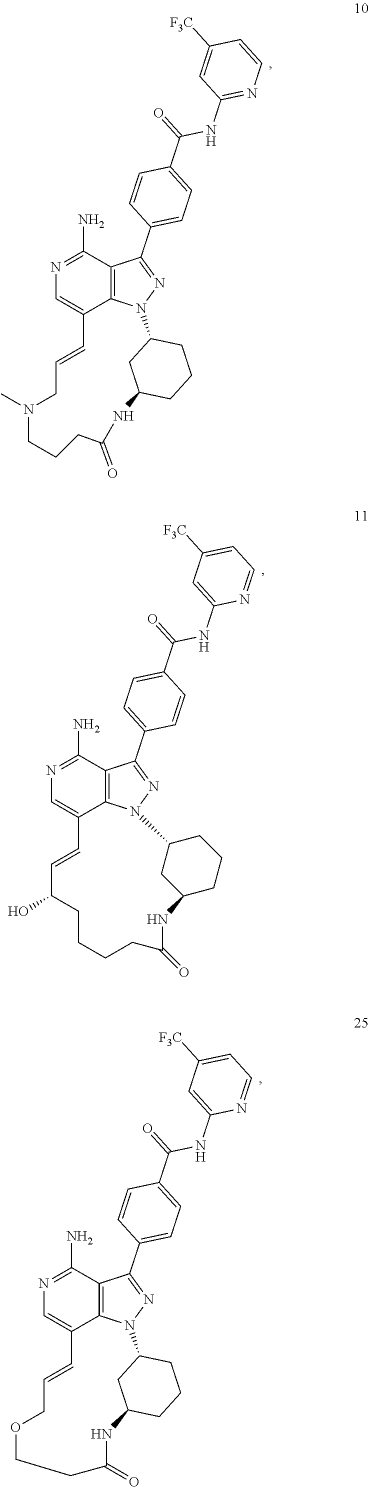 [SMILES: CN1C/C=C/c2cnc(N)c3c(-c4ccc(C(=O)Nc5cc(C(F)(F)F)ccn5)cc4)nn(c23)[C@@H]2CCC[C@H](C2)NC(=O)CCC1 ; Nc1ncc2c3c1c(-c1ccc(C(=O)Nc4cc(C(F)(F)F)ccn4)cc1)nn3[C@@H]1CCC[C@H](C1)NC(=O)CCCC[C@H](O)/C=C/2 ; Nc1ncc2c3c1c(-c1ccc(C(=O)Nc4cc(C(F)(F)F)ccn4)cc1)nn3[C@@H]1CCC[C@H](C1)NC(=O)CCOC/C=C/2]

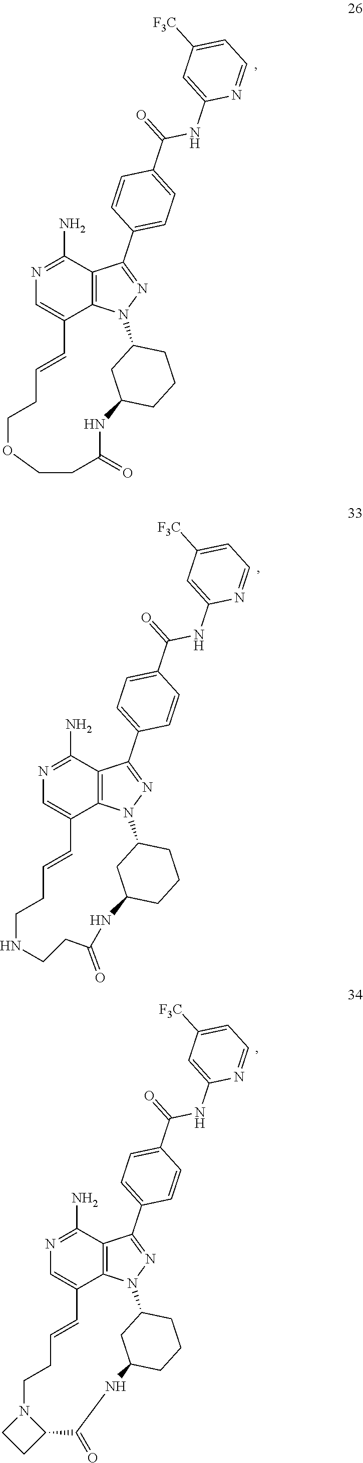 [SMILES: Nc1ncc2c3c1c(-c1ccc(C(=O)Nc4cc(C(F)(F)F)ccn4)cc1)nn3[C@@H]1CCC[C@H](C1)NC(=O)CCOCC/C=C/2 ; Nc1ncc2c3c1c(-c1ccc(C(=O)Nc4cc(C(F)(F)F)ccn4)cc1)nn3[C@@H]1CCC[C@H](C1)NC(=O)CCNCC/C=C/2 ; Nc1ncc2c3c1c(-c1ccc(C(=O)Nc4cc(C(F)(F)F)ccn4)cc1)nn3[C@@H]1CCC[C@H](C1)NC(=O)[C@@H]1CCN1CC/C=C/2]

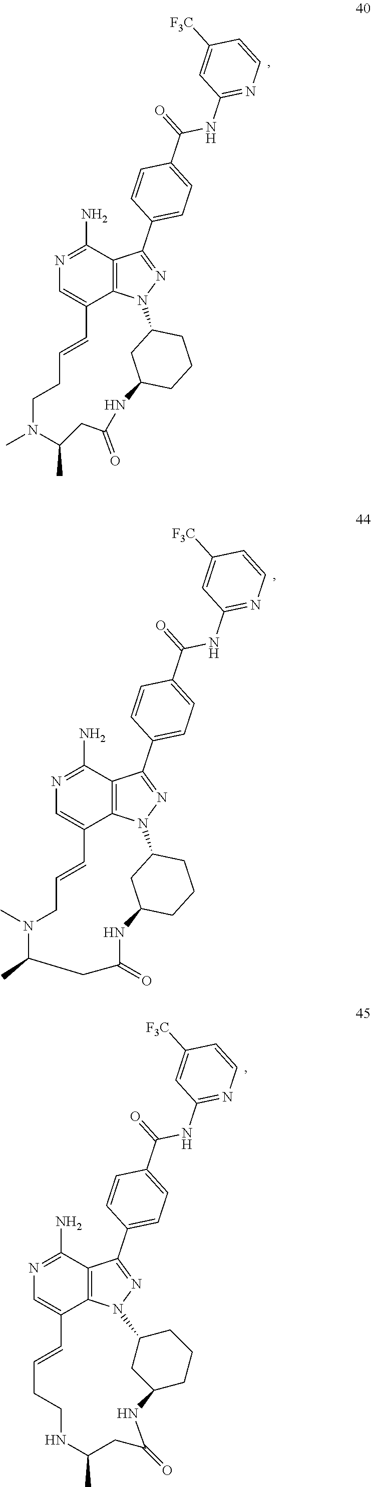 [SMILES: C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCN1C ; C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CN1C ; C[C@@H]1CC(=O)N[C@@H]2CCC[C@H](C2)n2nc(-c3ccc(C(=O)Nc4cc(C(F)(F)F)ccn4)cc3)c3c(N)ncc(c32)/C=C/CCN1]

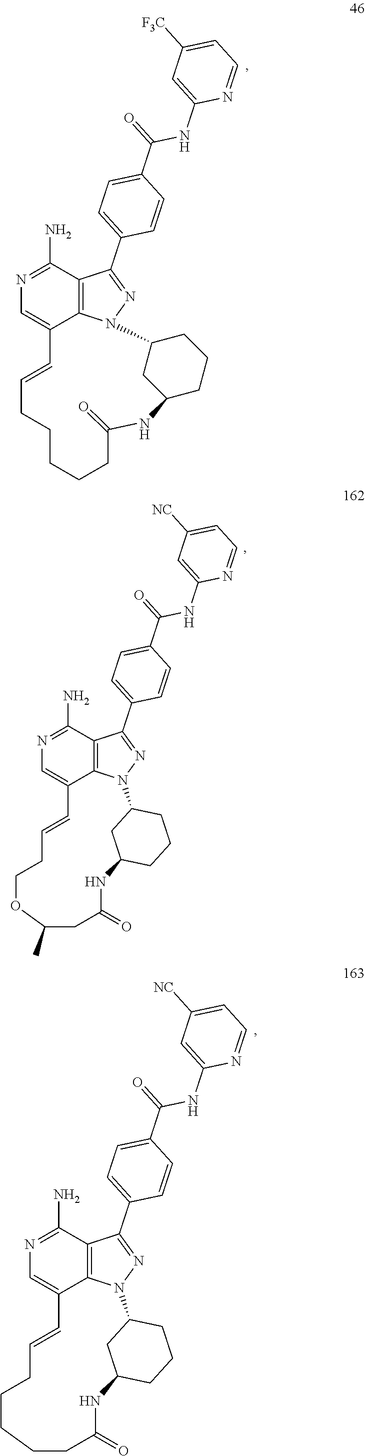 [SMILES: Nc1ncc2c3c1c(-c1ccc(C(=O)Nc4cc(C(F)(F)F)ccn4)cc1)nn3[C@@H]1CCC[C@H](C1)NC(=O)CCCCC/C=C/2 ; C[C@@H]1CC(=O)N[C@@H]2CCC[C@H](C2)n2nc(-c3ccc(C(=O)Nc4cc(C#N)ccn4)cc3)c3c(N)ncc(c32)/C=C/CCO1 ; N#Cc1ccnc(NC(=O)c2ccc(-c3nn4c5c(cnc(N)c35)/C=C/CCCCCC(=O)N[C@@H]3CCC[C@@H]4C3)cc2)c1]

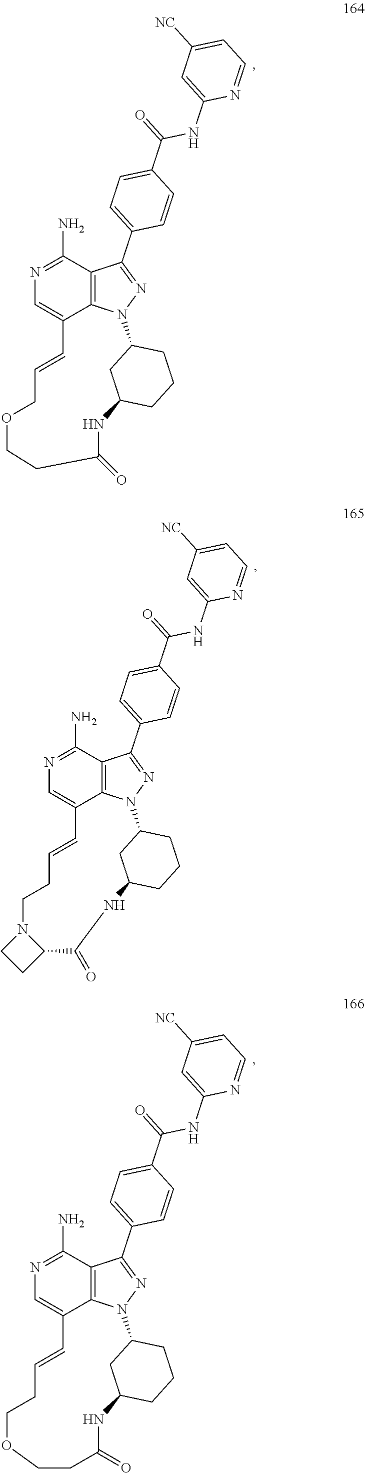 [SMILES: N#Cc1ccnc(NC(=O)c2ccc(-c3nn4c5c(cnc(N)c35)/C=C/COCCC(=O)N[C@@H]3CCC[C@@H]4C3)cc2)c1 ; N#Cc1ccnc(NC(=O)c2ccc(-c3nn4c5c(cnc(N)c35)/C=C/CCN3CC[C@H]3C(=O)N[C@@H]3CCC[C@@H]4C3)cc2)c1 ; N#Cc1ccnc(NC(=O)c2ccc(-c3nn4c5c(cnc(N)c35)/C=C/CCOCCC(=O)N[C@@H]3CCC[C@@H]4C3)cc2)c1]

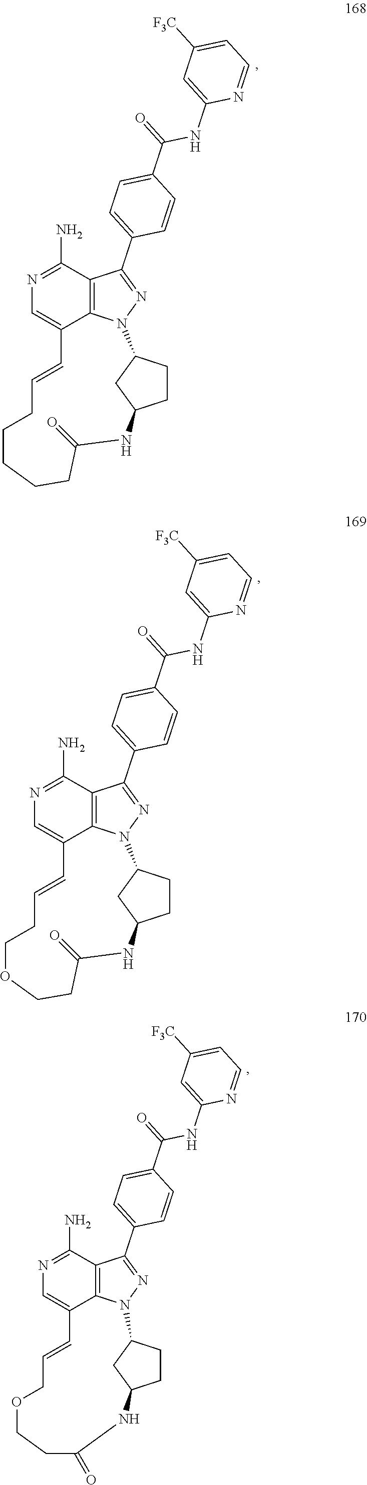 [SMILES: Nc1ncc2c3c1c(-c1ccc(C(=O)Nc4cc(C(F)(F)F)ccn4)cc1)nn3[C@@H]1CC[C@H](C1)NC(=O)CCCCC/C=C/2 ; Nc1ncc2c3c1c(-c1ccc(C(=O)Nc4cc(C(F)(F)F)ccn4)cc1)nn3[C@@H]1CC[C@H](C1)NC(=O)CCOCC/C=C/2 ; Nc1ncc2c3c1c(-c1ccc(C(=O)Nc4cc(C(F)(F)F)ccn4)cc1)nn3[C@@H]1CC[C@H](C1)NC(=O)CCOC/C=C/2]

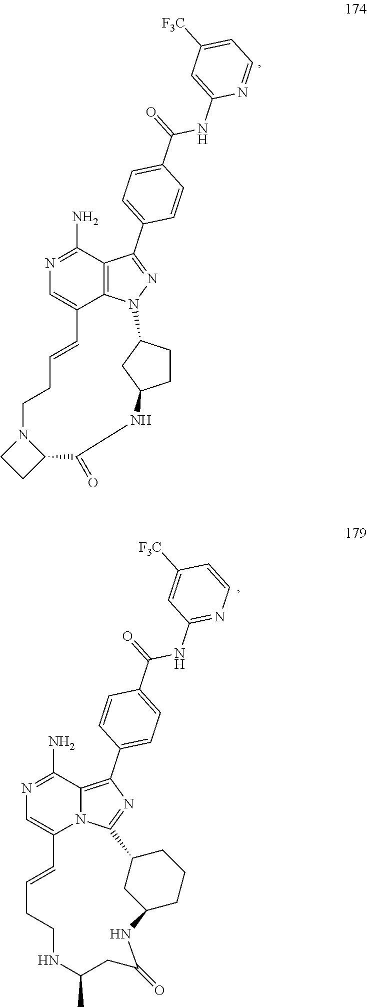 [SMILES: Nc1ncc2c3c1c(-c1ccc(C(=O)Nc4cc(C(F)(F)F)ccn4)cc1)nn3[C@@H]1CC[C@H](C1)NC(=O)[C@@H]1CCN1CC/C=C/2 ; C[C@@H]1CC(=O)N[C@@H]2CCC[C@H](C2)c2nc(-c3ccc(C(=O)Nc4cc(C(F)(F)F)ccn4)cc3)c3c(N)ncc(n23)/C=C/CCN1]

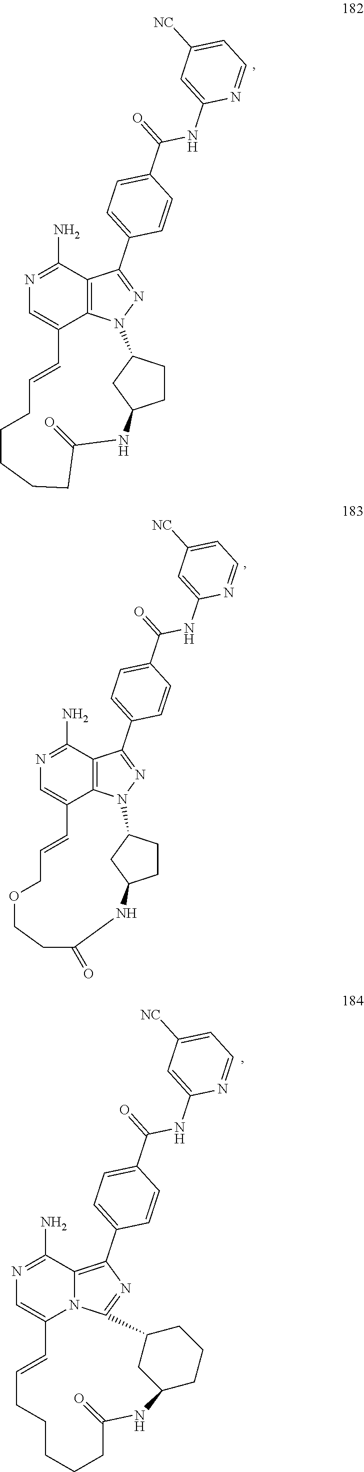 [SMILES: N#Cc1ccnc(NC(=O)c2ccc(-c3nn4c5c(cnc(N)c35)/C=C/CCCCCC(=O)N[C@@H]3CC[C@@H]4C3)cc2)c1 ; N#Cc1ccnc(NC(=O)c2ccc(-c3nn4c5c(cnc(N)c35)/C=C/COCCC(=O)N[C@@H]3CC[C@@H]4C3)cc2)c1 ; N#Cc1ccnc(NC(=O)c2ccc(-c3nc4n5c(cnc(N)c35)/C=C/CCCCCC(=O)N[C@@H]3CCC[C@@H]4C3)cc2)c1]

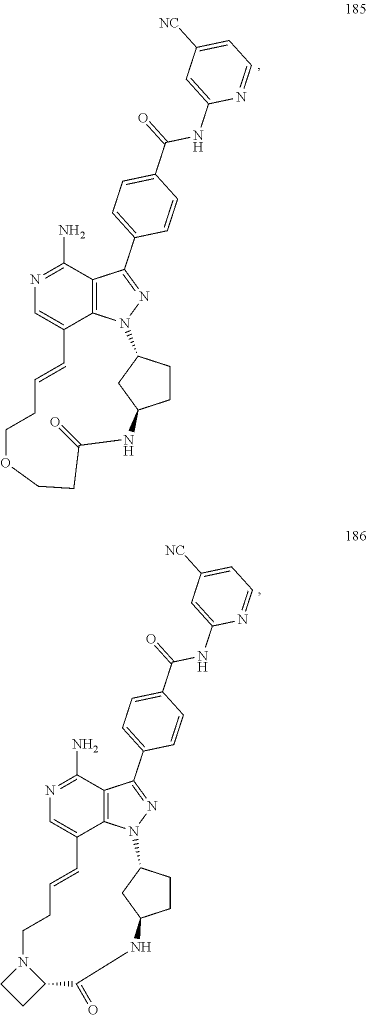 [SMILES: N#Cc1ccnc(NC(=O)c2ccc(-c3nn4c5c(cnc(N)c35)/C=C/CCOCCC(=O)N[C@@H]3CC[C@@H]4C3)cc2)c1 ; N#Cc1ccnc(NC(=O)c2ccc(-c3nn4c5c(cnc(N)c35)/C=C/CCN3CC[C@H]3C(=O)N[C@@H]3CC[C@@H]4C3)cc2)c1]

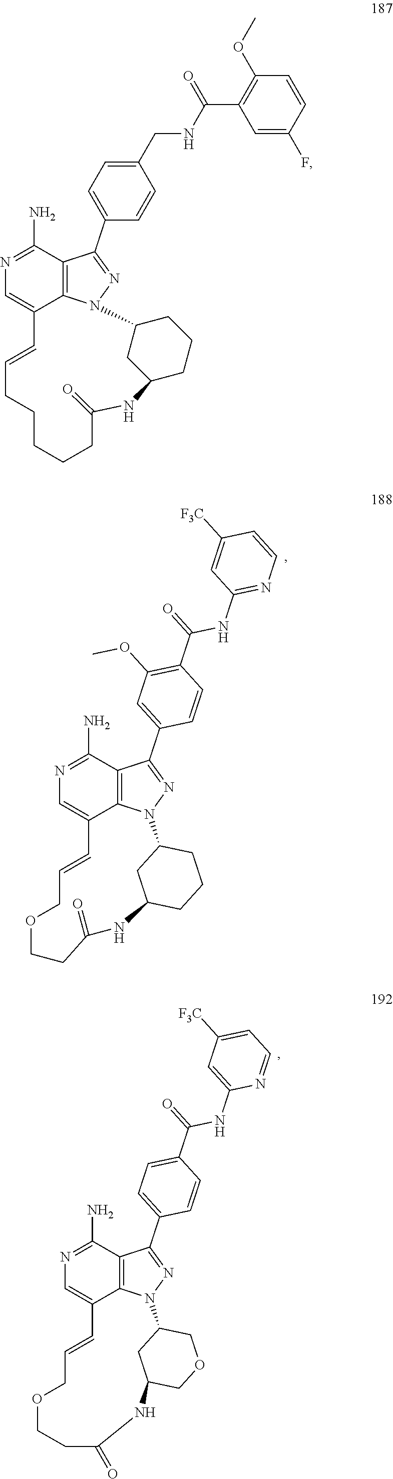 [SMILES: COc1ccc(F)cc1C(=O)NCc1ccc(-c2nn3c4c(cnc(N)c24)/C=C/CCCCCC(=O)N[C@@H]2CCC[C@@H]3C2)cc1 ; COc1cc(-c2nn3c4c(cnc(N)c24)/C=C/COCCC(=O)N[C@@H]2CCC[C@@H]3C2)ccc1C(=O)Nc1cc(C(F)(F)F)ccn1 ; Nc1ncc2c3c1c(-c1ccc(C(=O)Nc4cc(C(F)(F)F)ccn4)cc1)nn3[C@@H]1COC[C@H](C1)NC(=O)CCOC/C=C/2]

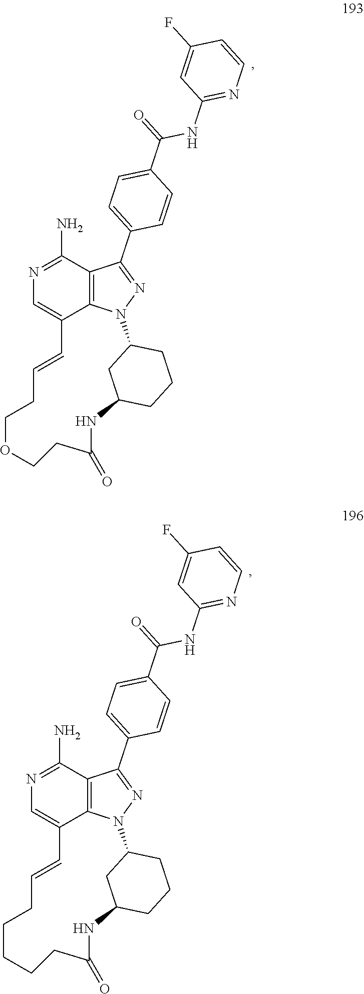 [SMILES: Nc1ncc2c3c1c(-c1ccc(C(=O)Nc4cc(F)ccn4)cc1)nn3[C@@H]1CCC[C@H](C1)NC(=O)CCOCC/C=C/2 ; Nc1ncc2c3c1c(-c1ccc(C(=O)Nc4cc(F)ccn4)cc1)nn3[C@@H]1CCC[C@H](C1)NC(=O)CCCCC/C=C/2]

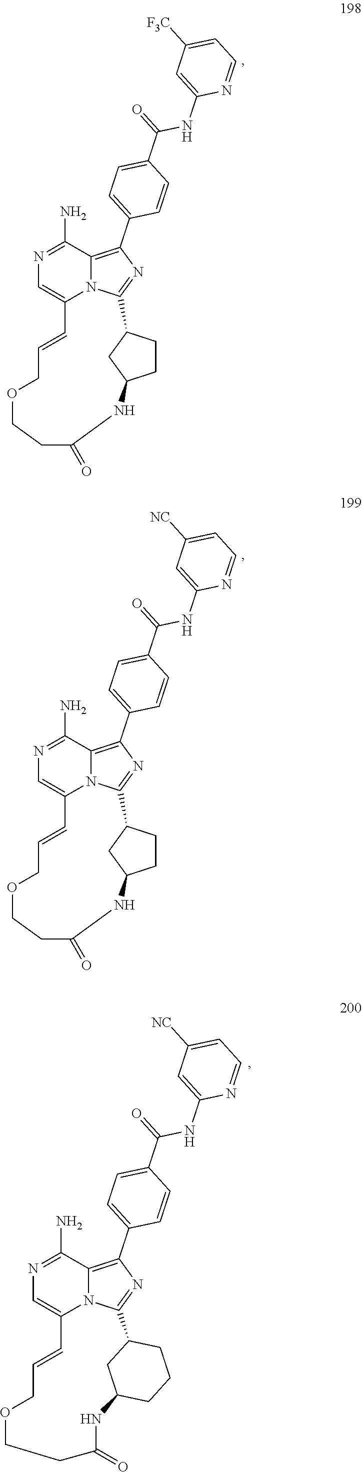 [SMILES: Nc1ncc2n3c(nc(-c4ccc(C(=O)Nc5cc(C(F)(F)F)ccn5)cc4)c13)[C@@H]1CC[C@H](C1)NC(=O)CCOC/C=C/2 ; N#Cc1ccnc(NC(=O)c2ccc(-c3nc4n5c(cnc(N)c35)/C=C/COCCC(=O)N[C@@H]3CC[C@@H]4C3)cc2)c1 ; N#Cc1ccnc(NC(=O)c2ccc(-c3nc4n5c(cnc(N)c35)/C=C/COCCC(=O)N[C@@H]3CCC[C@@H]4C3)cc2)c1]

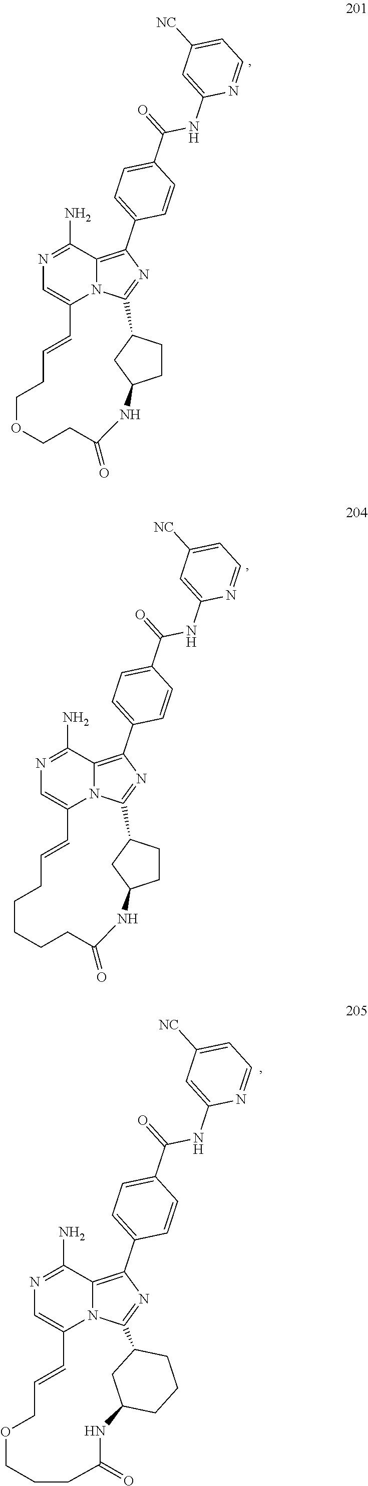 [SMILES: N#Cc1ccnc(NC(=O)c2ccc(-c3nc4n5c(cnc(N)c35)/C=C/CCOCCC(=O)N[C@@H]3CC[C@@H]4C3)cc2)c1 ; N#Cc1ccnc(NC(=O)c2ccc(-c3nc4n5c(cnc(N)c35)/C=C/CCCCCC(=O)N[C@@H]3CC[C@@H]4C3)cc2)c1 ; N#Cc1ccnc(NC(=O)c2ccc(-c3nc4n5c(cnc(N)c35)/C=C/COCCCC(=O)N[C@@H]3CCC[C@@H]4C3)cc2)c1]

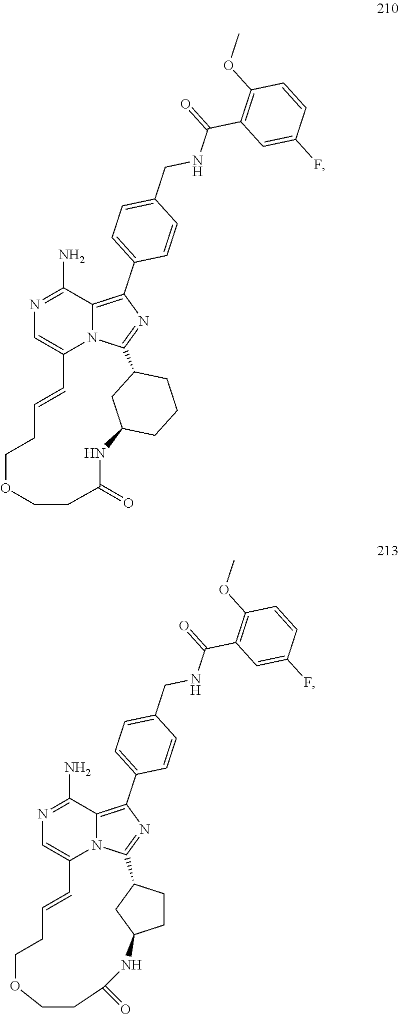 [SMILES: COc1ccc(F)cc1C(=O)NCc1ccc(-c2nc3n4c(cnc(N)c24)/C=C/CCOCCC(=O)N[C@@H]2CCC[C@@H]3C2)cc1 ; COc1ccc(F)cc1C(=O)NCc1ccc(-c2nc3n4c(cnc(N)c24)/C=C/CCOCCC(=O)N[C@@H]2CC[C@@H]3C2)cc1]

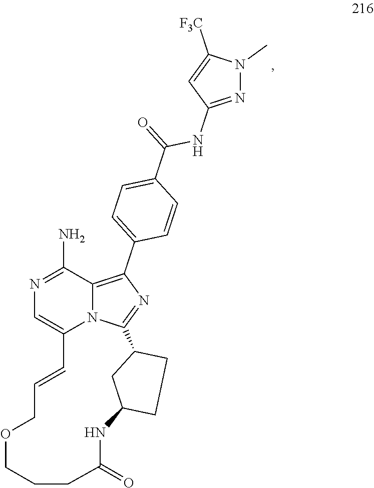 [SMILES: Cn1nc(NC(=O)c2ccc(-c3nc4n5c(cnc(N)c35)/C=C/COCCCC(=O)N[C@@H]3CC[C@@H]4C3)cc2)cc1C(F)(F)F]

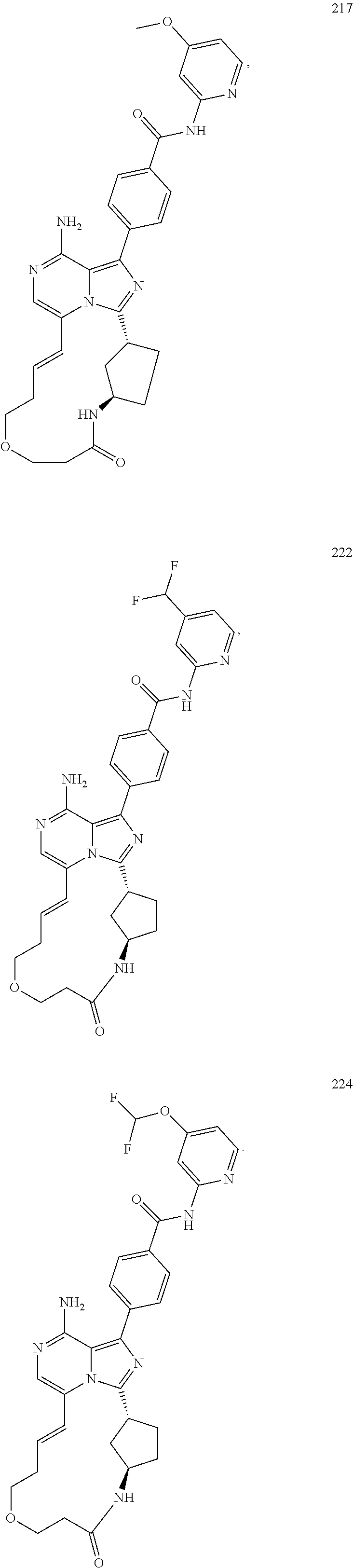 [SMILES: COc1ccnc(NC(=O)c2ccc(-c3nc4n5c(cnc(N)c35)/C=C/CCOCCC(=O)N[C@@H]3CC[C@@H]4C3)cc2)c1 ; Nc1ncc2n3c(nc(-c4ccc(C(=O)Nc5cc(C(F)F)ccn5)cc4)c13)[C@@H]1CC[C@H](C1)NC(=O)CCOCC/C=C/2 ; Nc1ncc2n3c(nc(-c4ccc(C(=O)Nc5cc(OC(F)F)ccn5)cc4)c13)[C@@H]1CC[C@H](C1)NC(=O)CCOCC/C=C/2]

40. A pharmaceutical composition which comprises the compound according to claim 32 or a pharmaceutically acceptable salt thereof and one or more pharmaceutically acceptable excipients.

41. A method for the treatment of Bruton's Tyrosine Kinase (BTK) mediated disorders, the method comprising administering to a subject in need thereof a compound according to claim 32 or a pharmaceutically acceptable salt thereof.

42. A method for the treatment of cancer, lymphoma, leukemia, or a disease selected from the group consisting of B-cell malignancy, B-cell lymphoma, diffuse large B-cell lymphoma, chronic lymphocyte leukemia, non-Hodgkin lymphoma for example ABC-DLBCL, mantle cell lymphoma, follicular lymphoma, hairy cell leukemia B-cell non-Hodgkin lymphoma, Waldenstrom's macroglobulinemia, Richter transformation, multiple myeloma, bone cancer, bone metastasis, chronic lymphocytic lymphomas, B-cell prolymphocyte leukemia, lymphoplasmacytic lymphoma, splenic marginal zone lymphoma, plasma cell lymphoma, plasmacytoma, extranodal marginal zone B-cell lymphoma, nodal marginal zone B-cell lymphoma, mediastinal (thymic) large B-cell lymphoma, intravascular large B-cell lymphoma, primary effusion lymphoma, Burkitt lymphoma/leukemia, lymphomatoid granulomatosis; or a disease selected from the group consisting of rheumatoid arthritis, psoriatic arthritis, infectious arthritis, progressive chronic arthritis, deforming arthritis, osteoarthritis, traumatic arthritis, gouty arthritis, Reiter's syndrome, polychondritis, acute synovitis and spondylitis, glomerulonephritis (with or without nephrotic syndrome), autoimmune hematologic disorders, hemolytic anemia, aplasic anemia, idiopathic thrombocytopenia, and neutropenia, autoimmune gastritis, and autoimmune inflammatory bowel diseases, ulcerative colitis, Crohn's disease, host versus graft disease, allograft rejection, chronic thyroiditis, Graves' disease, schleroderma, diabetes (type I and type II), active hepatitis (acute and chronic), pancreatitis, primary billiary cirrhosis, myasthenia gravis, multiple sclerosis, systemic lupus erythematosis, psoriasis, atopic dermatitis, contact dermatitis, eczema, skin sunburns, vasculitis (e.g. Behcet's disease) chronic renal insufficiency, Stevens-Johnson syndrome, inflammatory pain, idiopathic sprte, cachexia, sarcoidosis, Guillain-Barré syndrome, uveitis, conjunctivitis, kerato conjunctivitis, otitis media, periodontal disease, pulmonary interstitial fibrosis, asthma, bronchitis, rhinitis, sinusitis, pneumoconiosis, pulmonary insufficiency syndrome, pulmonary emphysema, pulmonary fibrosis, silicosis, chronic inflammatory pulmonary disease, and chronic obstructive pulmonary disease, wherein the method comprises administering to a subject in need thereof a compound according to claim 32 or a pharmaceutically acceptable salt thereof.
